(19) United States
(12) Patent Application Publication
(10) Pub. No.: US 2025/0276966 A1
(43) Pub. Date: Sep. 4, 2025

(54) Nitrogen-Linked Benzisoxazole Sulfonamide Derivatives

(71) Applicants: Pfizer Inc., New York, NY (US); CTXT PTY LTD, Melbourne (AU)

(72) Inventors: Alexander Burtea, Ladera Ranch, CA (US); Michael Raymond Collins, San Diego, CA (US); Matthew L. Del Bel, Millbrae, CA (US); Samantha Elizabeth Greasley, San Diego, CA (US); Eric Johnson, San Diego, CA (US); Ted William Johnson, Carlsbad, CA (US); Robert Arnold Kumpf, Carlsbad, CA (US); Pei-Pei Kung, San Diego, CA (US); Timothy Patrick Montgomery, San Diego, CA (US); Sacha Ninkovic, La Jolla, CA (US); Gwenaella Christine Rescourio, San Diego, CA (US); Paul Francis Richardson, San Diego, CA (US); Stephanie Anne Scales, San Diego, CA (US); Scott Channing Sutton, San Diego, CA (US)

(21) Appl. No.: 19/210,024

(22) Filed: May 16, 2025

Related U.S. Application Data
(63) Continuation of application No. 18/903,849, filed on Oct. 1, 2024.
(60) Provisional application No. 63/697,154, filed on Sep. 20, 2024.
(60) Provisional application No. 63/665,990, filed on Jun. 28, 2024.
(60) Provisional application No. 63/633,660, filed on Apr. 12, 2024.
(60) Provisional application No. 63/566,213, filed on Mar. 15, 2024.
(60) Provisional application No. 63/546,442, filed on Oct. 30, 2023.
(60) Provisional application No. 63/542,051, filed on Oct. 2, 2023.

(51) Int. Cl. C07D 413/14; A61K 31/423; A61K 31/4439; A61K 31/454; A61K 31/501; A61K 31/506; A61K 31/519; A61K 31/5377; C07D 413/12; C07D 471/04

(52) U.S. Cl. CPC C07D 413/14; A61K 31/423; A61K 31/4439; A61K 31/454; A61K 31/501; A61K 31/506; A61K 31/519; A61K 31/5377; C07D 413/12; C07D 471/04

(57) ABSTRACT

The present invention relates to compounds of Formula (I):

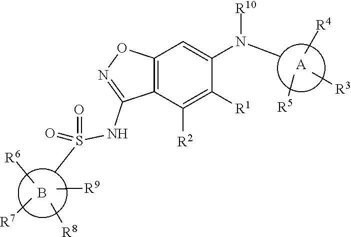

or pharmaceutically acceptable salts thereof, wherein Ring A, Ring B, R¹-R¹⁶, n and p are defined herein. The novel nitrogen-linked benzisoxazole sulfonamide derivatives are useful in the treatment of abnormal cell growth, such as cancer, in patients. Additional embodiments relate to pharmaceutical compositions containing the compounds and to methods of using the compounds and compositions in the treatment of abnormal cell growth, such as cancer, in patients.

This application claims the benefit of U.S. Provisional Application No. 63/542,051 filed Oct. 2, 2023, U.S. Provisional Application No. 63/546,442 filed Oct. 30, 2023, U.S. Provisional Application No. 63/566,213 filed Mar. 15, 2024, U.S. Provisional Application No. 63/633,660 filed Apr. 12, 2024, U.S. Provisional Application No. 63/665,990 filed Jun. 28, 2024, and U.S. Provisional Application No. 63/697,154 filed Sep. 20, 2024, the contents of which are hereby incorporated by reference in their entireties.

### SEQUENCE LISTING

This application includes an electronically submitted sequence listing in .xml format. The .xml file contains a sequence listing entitled “PC073303A_SEQListing_ST26.xml” created on May 16, 2025 and having a size of 20,325 bytes. The sequence listing contained in this .xml file is part of the specification and is herein incorporated by reference in its entirety.

### BACKGROUND OF THE INVENTION

The present invention relates to novel nitrogen-linked benzisoxazole sulfonamide derivatives, which act as Lysine Acetyl Transferase (KAT) inhibitors of the MYST family and may be useful in the treatment of abnormal cell growth, such as cancer, in patients. The present invention also relates to pharmaceutical compositions containing the compounds and to methods of using the compounds and compositions in the treatment of abnormal cell growth in patients.

KAT enzymes perform important regulatory functions in cancer and are therefore frequently targeted by mutations, translocations, and amplifications (Hu, Z., et al., Genomic characterization of genes encoding histone acetylation modulator proteins identifies therapeutic targets for cancer treatment. Nat Commun. 2019 Feb. 13; 10 (1):733). KAT6A was identified in 1996 as part of a chromosomal translocation t(8; 16) (p11; p13) with CREBBP (CREB-binding protein) in a subtype of acute myeloid leukemia (AML) (Borrow, J., et al, The translocation t(8; 16)(p11; p13) of acute myeloid leukaemia fuses a putative acetyltransferase to the CREB-binding protein. Nat. Genet. 1996, 14, 33-41). Additional KAT6A and KAT6B translocations were subsequently identified in more AML patients, generating fusions with other HATs such as EP300 (adenoviral EIA-associated protein p300), NCOA2 (nuclear receptor coactivator 2), and NCOA3 (Huang, et al.).

In human carcinomas, especially breast cancer, KAT6A was identified as a part of the recurrently amplified region of 8p11-12 found in 10-15% of breast cancers (Adélaïde J., et al, Chromosome region 8p11-p21: refined mapping and molecular alterations in breast cancer. Genes Chromosomes Cancer. 1998 July; 22 (3):186-99). Breast cancer cell lines harboring KAT6A amplifications over-express KAT6A, implicating KAT6A as a putative breast cancer susceptibility gene. Turner-Ivey et al. utilized a genome-scale shRNA screening strategy to identify KAT6A as a significant dependency in 8p11 amplified breast cancer cell lines harboring overexpression of KAT6A (Turner-Ivey B, et al., KAT6A, a chromatin modifier from the 8p11-p12 amplicon is a candidate oncogene in luminal breast cancer. Neoplasia. 2014 August; 16 (8):644-55). Yu et al. subsequently demonstrated in 8p11 amplified breast cancer cells that KAT6A localized to the estrogen receptor promoter and that shRNA-mediated knockdown of KAT6A reduced ESR1 mRNA and protein levels of ERα (Yu, L., et al., Identification of MYST3 as a novel epigenetic activator of ERα frequently amplified in breast cancer. Oncogene 2017, 36, 2910-2918). Moreover, they showed that the growth defect resulting from KAT6A depletion in 8p11 amplified breast cancer cells was partially rescued by re-expression of ESR1. These findings indicate an important role of KAT6A in gene regulation of ERα required for growth of ER+ breast cancer cells.

Chromosome 8p11-12 amplifications and KAT6A over-expression exists in additional tumor types including ovarian cancer, uterine cervix cancer, lung adenocarcinoma, colon and rectal adenocarcinomas, and medulloblastoma (Zack T I, et al., Pan-cancer patterns of somatic copy number alteration. Nat Genet 2013 45:1134-1140; Northcott P A, et al., Multiple recurrent genetic events converge on control of histone lysine methylation in medulloblastoma. Nat Genet 2009 41:465-472). Additional KAT6A tumor dependencies including prostate cancer have been identified (Yu C, et al., High-throughput identification of genotype-specific cancer vulnerabilities in mixtures of barcoded tumor cell lines. Nat Biotechnol. 2016; Meyers R M, et al. Computational correction of copy number effect improves specificity of CRISPR-Cas9 essentiality screens in cancer cells. Nat Genet. 2017; and Tsherniak A, et al., Defining a cancer dependency map. Cell. 2017). Overall, these data demonstrate the broader therapeutic opportunity for targeting KAT6A in additional tumor types.

In addition to its catalytic function mediated by the histone acetyltransferase (HAT) domain the KAT6A protein includes additional domains such as PHD domains, an acidic domain, and a serine/methionine-rich domain. KAT6A regulation of gene expression independent of its catalytic activity has been reported (Kitabayashi, I., et al., Activation of AML1 mediated transcription by MOZ and inhibition by the MOZ-CBP fusion protein. EMBO J. 2001, 20(24): 7184-7196). The dependence of ER+ breast cancer cells on KAT6A was demonstrated using RNA interference to knockdown KAT6A protein level (Turner-Ivey B., et al. and Yu, L., et al.) However, the requirement of KAT6A catalytic activity for ERα expression and ER+ breast cancer cell proliferation is unclear.

KAT7 (HBO1/MYST2) is a member of the MYST (MOZ, Ybf2/Sas3, Sas2, and Tip60) family of histone lysine acetyltransferases that target histone and non-histone proteins for lysine acetylation (Neal, Pannuti et al. 2000, Roth, Denu et al. 2001, Yang 2004, Sapountzi and Cote 2011, Wang and Cole 2020). KAT7 exists as the enzymatic component of a four member protein complex comprised of alternative adaptor proteins that include JADE1/2/3 or BRPF1/2/3 as well as ING family proteins together with MEAF6 (Doyon, Cayrou et al. 2006, Lalonde, Avvakumov et al. 2013). Post-translational modification of histone tails is a major mechanism for regulation of chromatin structure and function and has been shown to play critical roles in transcription, DNA damage repair, and DNA replication (Jenuwein and Allis 2001, Suganuma and Workman 2011, Allis and Jenuwein 2016). Enzymatically, KAT7 transfers the acetyl group from acetyl-CoA to the e-amino group of lysine residues CoA (Roth, Denu et al. 2001). KAT7 can accommodate additional acyl-CoA co-factors to catalyze histone propionylation, butyrylation, crotonylation, but not succinylation (Xiao, Li et al. 2021). The formation of KAT7 protein complexes with different scaffold proteins, BPRF1/2/3 or JADE1/2/3, regulates biochemical activity and substrate presence for histone H3 or histone H4, respectively (Doyon, Cayrou et al. 2006, Foy, Song et al. 2008, Kueh, Dixon et al. 2011, Mishima, Miyagi et al. 2011, Lalonde, Avvakumov et al. 2013, Tao, Zhong et al. 2017).

Additional complex subunits ING4/ING5 and MEAF6 regulate the recruitment and function of KAT7 complexes on chromatin through interactions with modified histone proteins (Doyon, Cayrou et al. 2006, Champagne, Saksouk et al. 2008, Saksouk, Avvakumov et al. 2009, Palacios, Moreno et al. 2010, Matsuura, Tani et al. 2020, Barman, Roy et al. 2022). The combinatorial function of protein reader domains on KAT7-interacting proteins including PHD domain (Doyon, Cayrou et al. 2006, Saksouk, Avvakumov et al. 2009, Klein, Muthurajan et al. 2016), PWWP domain (Zhang, Lei et al. 2021), BROMO domains (Filippakopoulos and Knapp 2012, Barman, Roy et al. 2022), provides specificity for positioning of KAT7 complexes on chromatin.

KAT7 was first identified as a protein binding to ORC1, the largest subunit of origin recognition complex involved in DNA replication (lizuka and Stillman 1999). The zinc finger of KAT7 interacts with MCM2, a key component of the pre-replication complex, in cervical carcinoma cells (Burke, Cook et al. 2001, Doyon, Cayrou et al. 2006). KAT7 has been reported to interact directly with CDT1, where it enhances CDT1-dependent re-replication at replication DNA origins (Miotto and Struhl 2008), indicating a crucial role of KAT7 during both initiation and elongation of DNA synthesis. Chromatin-immunoprecipitation DNA sequencing (ChIP-seq) identified KAT7 binding at DNA replication origins (Feng, Vlassis et al. 2016, Xiao, Li et al. 2021). Consistent with the role of KAT7 in DNA replication, knockdown of KAT7 using siRNA leads to growth arrest in cancer cell lines enriching is the S phase of the cell cycle (Doyon, Cayrou et al. 2006, Wu and Liu 2008).

Apart from its proposed role in DNA replication, there is evidence that KAT7 is involved in gene regulation through regulation of transcription. KAT7 complexes bind at transcriptional start sites and gene coding regions (Saksouk, Avvakumov et al. 2009, Xiao, Li et al. 2021). In complex with nuclear receptors such as progesterone receptor, the KAT7 MYST domain can function as a co-activator to increase expression of target genes (Georgiakaki, Chabbert-Buffet et al. 2006). KAT7 also interacts with androgen receptor in a ligand-dependent manner and can lead to both activation and repression of AR target gene expression, indicating KAT7 can function both as a transcriptional activator and repressor (Sharma, Zarnegar et al. 2000, Mi, Ji et al. 2023). Expression of the N-terminal serine-rich region of KAT7 inhibits NF-kappaB activity stimulated by TNFalpha in 293T cells through sequestration of co-factor binding, indicating KAT7 non-catalytic function can also impact transcriptional regulation (Contzler, Regamey et al. 2006).

During mouse development, KAT7 acts as an essential activator of patterning genes gene expression during postgastrulation embryonic development. Knockout of the KAT7 gene in mouse embryos leads to increased apoptosis, particularly affecting mesodermal structures and embryonic lethality at E10.5 (Kueh, Dixon et al. 2011). Conditional knockout of KAT7 in developing mouse indicates KAT7 performs functions in additional tissues during development including blood vessel endothelial cells (Grant, Hickey et al. 2021), bone marrow and fetal liver hematopoiesis (Mishima, Miyagi et al. 2011, Yang, Kueh et al. 2022), T cells (Newman, Voss et al. 2017), and neurons and oligodendrocytes (Kueh, Bergamasco et al. 2023). The Drosophila homologue of KAT7, chameau, is essential for larvae development. Heterozygous knockout alleles for chameau in developing embryos display defects in homeotic transformations mediated by derepression in Hox gene expression (Grienenberger, Miotto et al. 2002).

Expression of KAT7 has been found to be elevated in a number of cancers including esophageal carcinomas, bladder, testicular, breast, ovarian, and gastric cancer (lizuka, Takahashi et al. 2009, Chen, Zhou et al. 2018, Wang, Chen et al. 2019, Guo, Li et al. 2022). KAT7 was identified as a common genomic amplified region on chromosome 17q21 in ER+ and HER2+ breast cancer (Hu, Stern et al. 2009). KAT7 modulates estrogen receptor-dependent transcription and can directly interact and acetylate estrogen receptor alpha (ERα) leading to decreased protein stability (lizuka, Susa et al. 2013). Inhibition of KAT7 in breast cancer cell lines leads to loss of cell proliferation and blocks progression through the S phase of the cell cycle (Hu, Stern et al. 2009). Conversely, overexpression of KAT7 causes an increase in colony formation on soft agar in breast cancer cell lines (Hu, Stern et al. 2009). In breast cancer stem-like cells phosphorylation of KAT7 by the CDK2/Cyclin E complex is enriched in a CD44hi/CD24lo population (Duong, Akli et al. 2013). KAT7 over-expression in breast cancer increases Phosphatidylinositol-4,5-bisphosphate 3-kinase catalytic subunit alpha (PIK3CA) transcription, leading to enhanced PI3K/AKT signaling and resistance to radiotherapy (Ma, Chen et al. 2023).

KAT7 acetyltransferase active has been linked with cancer pathways. KAT7 and associated complex members ING4 and ING5 physically interact with p53 tumor suppressor gene (Shiseki, Nagashima et al. 2003, lizuka, Sarmento et al. 2008). Inhibition of KAT7 leads to downregulation of a large number of genes linked to the p53 pathway of cell cycle control, senescence, and apoptosis (Avvakumov, Lalonde et al. 2012). KAT7 promoted bladder cancer cells proliferation via activation the Wnt/B-catenin signaling pathway (Chen, Zhou et al. 2018). KAT7 promoted the transcription and nuclear translocation of Yes-associated protein 1 (YAP1) through over-expression in gastric cancer (Guo, Li et al. 2022).

In AML, KAT7 is over-expressed in leukemia stem cells, facilitating expression of HOXA9 and HOXA10, sustaining the functional properties of leukemia stem cells. In MLL rearranged AML, KAT7 acetylation of histones recruitments of MLL-fusion-associated adaptor proteins such as BRD4 and AF4 to gene promoters (Au, Gu et al. 2021, Takahashi, Kanai et al. 2021). KAT7 knockdown or small molecule inhibition cause reduce leukemia burden in mouse models (Sauer, Arteaga et al. 2015, MacPherson, Anokye et al. 2020, Au, Gu et al. 2021). An oncogene fusion protein, nucleoporin-98 (NUP98)-KAT7 has been identified in Chronic myelomonocytic leukemia (CMML). Experimental over-expression of NUP98-KAT7 in mouse models leads to over-expression of HOXA9 and induces a leukemogenesis (Hayashi, Harada et al. 2019).

In light of the established role of KATs in general, and MYSTs in particular, in diseases such as cancer, a need exists for new inhibitors of these proteins.

### SUMMARY OF THE INVENTION

The present invention provides, in part, compounds of Formula (I) and pharmaceutically acceptable salts thereof. Such compounds may be useful in the treatment of cancer. Also provided are pharmaceutical compositions comprising the compounds or salts of the invention, alone or in combination with additional therapeutic agents. The present invention also provides, in part, methods for preparing such compounds, pharmaceutically acceptable salts and compositions of the invention, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to a first embodiment of the invention there is provided a compound of Formula (I):

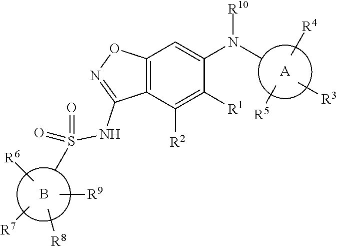

or a pharmaceutically acceptable salt thereof, wherein:
R¹ is hydrogen, methoxy, or fluoro;
R² is hydrogen or methoxy,
provided that R¹ and R² are not both hydrogen,
further provided that R¹ and R² are not both methoxy;
Ring A is C₄-C₅ cycloalkyl, 4-6 membered heterocycloalkyl, or 5-10 membered heteroaryl, wherein the point of attachment is at a carbon atom;
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

According to a second embodiment of the invention there is provided a compound of Formula (Ia):

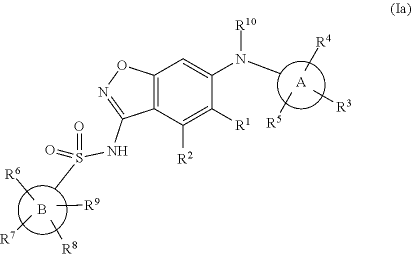

or a pharmaceutically acceptable salt thereof, wherein:
R¹ is hydrogen, methoxy, or fluoro;
R² is hydrogen or methoxy,
provided that R¹ and R² are not both hydrogen,
further provided that R¹ and R² are not both methoxy;
Ring A is C₄-C₅ cycloalkyl, 4-6 membered heterocycloalkyl, or 5-10 membered heteroaryl, wherein the point of attachment is at a carbon atom;
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)n-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

According to a third embodiment of the invention there is provided a compound of Formula (Ib):

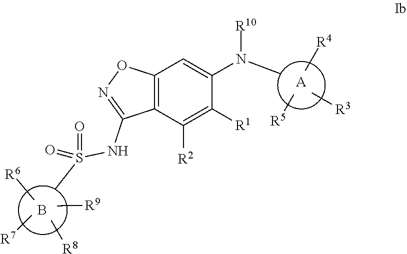

or a pharmaceutically acceptable salt thereof, wherein:
R¹ is hydrogen, methoxy, or fluoro;
R² is hydrogen or methoxy,
provided that R¹ and R² are not both hydrogen,
further provided that R¹ and R² are not both methoxy;
Ring A is C₄-C₅ cycloalkyl, 4-6 membered heterocycloalkyl, or 5-10 membered heteroaryl, wherein the point of attachment is at a carbon atom;
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

Described below are embodiments of the invention, where for convenience Embodiment 1 (E1), Embodiment 2 (E2), and Embodiment 3 (E3) are identical to the embodiments of Formula (I), Formula (Ia) and Formula (Ib) provided above.

It is to be understood that both the foregoing general description and the following detailed description are exemplary and explanatory only and are not restrictive of the invention, as claimed.

### BRIEF DESCRIPTION OF THE DRAWINGS

FIG. 1 shows an X-ray crystal structure of 2,6-dimethoxy-4-[(2S)-oxolan-2-yl]benzene-1-sulfonyl chloride (Intermediate 140h) demonstrating absolute stereochemistry of an(S) configuration for 2,6-dimethoxy-4-[(2S)-oxolan-2-yl]benzene-1-sulfonyl chloride (Intermediate 140h).

FIG. 2 shows an X-ray crystal structure of (2R)-2-(4-bromo-3,5-dimethoxyphenyl)-1,4-dioxane (Intermediate 145a) demonstrating absolute stereochemistry of an (R) configuration for (2R)-2-(4-bromo-3,5-dimethoxyphenyl)-1,4-dioxane (Intermediate 145a).

FIG. 3 shows an X-ray crystal structure of N-{6-[(5-cyclopropyl-1H-pyrazol-5-yl)amino]-5-methoxy-1,2-benzoxazol-3-yl}-4-[(2S)-1,4-dioxan-2-yl]-2,6-dimethoxybenzene-1-sulfonamide (Example 146) demonstrating absolute stereochemistry of an(S) configuration for N-{6-[(5-cyclopropyl-1H-pyrazol-5-yl)amino]-5-methoxy-1,2-benzoxazol-3-yl}-4-[(2S)-1,4-dioxan-2-yl]-2,6-dimethoxybenzene-1-sulfonamide (Example 146).

FIG. 4 shows an X-ray crystal structure of N-{6-[(5-cyclopropyl-1H-pyrazol-5-yl)amino]-5-methoxy-1,2-benzoxazol-3-yl}-2,6-dimethoxy-4-[(2S)-1-methylpyrrolidin-2-yl]benzene-1-sulfonamide (Example 189) demonstrating absolute stereochemistry of an(S) configuration for N-{6-[(5-cyclopropyl-1H-pyrazol-5-yl)amino]-5-methoxy-1,2-benzoxazol-3-yl}-2,6-dimethoxy-4-[(2S)-1-methylpyrrolidin-2-yl]benzene-1-sulfonamide (Example 189).

### DETAILED DESCRIPTION OF THE INVENTION

The present invention may be understood more readily by reference to the following detailed description of the embodiments of the invention and the Examples included herein. It is to be understood that this invention is not limited to specific synthetic methods of making that may of course vary. It is to be also understood that the terminology used herein is for the purpose of describing specific embodiments only and is not intended to be limiting.

E1 A compound of Formula (I) or a pharmaceutically acceptable salt thereof, as defined above.

E2 A compound of Formula (Ia) or a pharmaceutically acceptable salt thereof, as defined above.

E3 A compound of Formula (Ib) or a pharmaceutically acceptable salt thereof, as defined above.

E4 The compound of any one of embodiments E1 to E3, or a pharmaceutically acceptable salt thereof, wherein Ring A is selected from the group consisting of cyclobutyl, cyclopentyl, oxetanyl, tetrahydrofuranyl, tetrahydropyranyl, pyrrolyl, imidazolyl, isoxazolyl, oxazolyl, 1-oxa-2,4-diazolyl, triazolyl, pyrazolyl, pyridinyl, pyridazinyl, pyrimidinyl, pyrazinyl, 5,6-dihydro-4H-pyrrolo[1,2-b]pyrazolyl, 5′,6′-dihydrospiro[cyclopropane-1,4′-pyrrolo[1,2-b]pyrazolyl], and 2,4,5,6-tetrahydropyrrolo[3,4-c]pyrazolyl.

E5 The compound of embodiment E4, or a pharmaceutically acceptable salt thereof, wherein Ring A is cyclobutyl.

E6 The compound of embodiment E4, or a pharmaceutically acceptable salt thereof, wherein Ring A is cyclopentyl.

E7 The compound of embodiment E4, or a pharmaceutically acceptable salt thereof, wherein Ring A is oxetanyl.

E8 The compound of embodiment E4, or a pharmaceutically acceptable salt thereof, wherein Ring A is tetrahydrofuranyl.

E9 The compound of embodiment E4, or a pharmaceutically acceptable salt thereof, wherein Ring A is tetrahydropyranyl.

E10 The compound of embodiment E4, or a pharmaceutically acceptable salt thereof, wherein Ring A is pyrrolyl.

E11 The compound of embodiment E4, or a pharmaceutically acceptable salt thereof, wherein Ring A is imidazolyl.

E12 The compound of embodiment E4, or a pharmaceutically acceptable salt thereof, wherein Ring A is isoxazolyl.

E13 The compound of embodiment E4, or a pharmaceutically acceptable salt thereof, wherein Ring A is oxazolyl.

E14 The compound of embodiment E4, or a pharmaceutically acceptable salt thereof, wherein Ring A is 1-oxa-2,4-diazolyl.

E15 The compound of embodiment E4, or a pharmaceutically acceptable salt thereof, wherein Ring A is triazolyl.

E16 The compound of embodiment E4, or a pharmaceutically acceptable salt thereof, wherein Ring A is triazolyl; R⁶ is hydrogen or C₁₋₃ alkyl; and each of R⁷, R⁸, and R⁹ are hydrogen.

E17 The compound of embodiment E4, or a pharmaceutically acceptable salt thereof, wherein Ring A is pyrazolyl.

E18 The compound of embodiment E4, or a pharmaceutically acceptable salt thereof, wherein Ring A is pyridinyl.

E19 The compound of embodiment E4, or a pharmaceutically acceptable salt thereof, wherein Ring A is pyridazinyl.

E20 The compound of embodiment E4, or a pharmaceutically acceptable salt thereof, wherein Ring A is pyrimidinyl.

E21 The compound of embodiment E4, or a pharmaceutically acceptable salt thereof, wherein Ring A is pyrazinyl.

E22 The compound of embodiment E4, or a pharmaceutically acceptable salt thereof, wherein Ring A is 5,6-dihydro-4H-pyrrolo[1,2-b]pyrazolyl.

E23 The compound of embodiment E4, or a pharmaceutically acceptable salt thereof, wherein Ring A is 5′,6′-dihydrospiro[cyclopropane-1,4′-pyrrolo[1,2-b]pyrazolyl].

E24 The compound of embodiment E4, or a pharmaceutically acceptable salt thereof, wherein Ring A is 2,4,5,6-tetrahydropyrrolo[3,4-c]pyrazolyl.

E25 A compound of Formula (II):

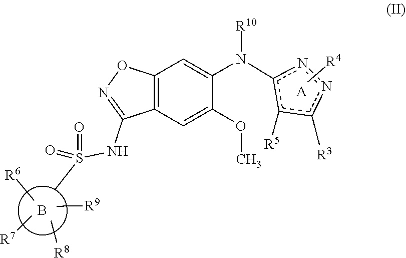

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E26 A compound of Formula (IIa):

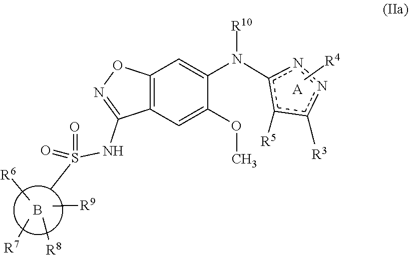

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E27 A compound of Formula (IIb):

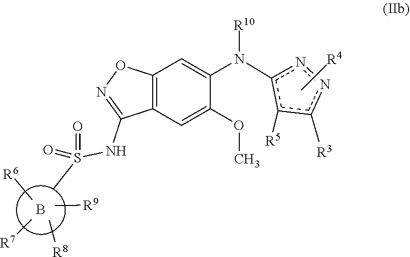

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
Ring B is C₃-C₆ cycloalkyl, C₆-C₁₀ aryl, or 5-10 membered heteroaryl;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E28 The compound any one of embodiments E1 to E27, or a pharmaceutically acceptable salt thereof, wherein Ring B is cyclopropyl, 1,2,3,4-tetrahydronaphthyl, naphthyl, chromanyl, isochromanyl, 2,3-dihydrobenzo[b][1,4]dioxinyl, pyrazolyl, pyrimidinyl, quinolinyl, or indazolyl.

E29 The compound of embodiment E28, or a pharmaceutically acceptable salt thereof, wherein Ring B is cyclopropyl.

E30 The compound of embodiment E28, or a pharmaceutically acceptable salt thereof, wherein Ring B is 1,2,3,4-tetrahydronaphthyl.

E31 The compound of embodiment E28, or a pharmaceutically acceptable salt thereof, wherein Ring B is naphthyl.

E32 The compound of embodiment E28, or a pharmaceutically acceptable salt thereof, wherein Ring B is chromanyl.

E33 The compound of embodiment E28, or a pharmaceutically acceptable salt thereof, wherein Ring B is isochromanyl.

E34 The compound of embodiment E28, or a pharmaceutically acceptable salt thereof, wherein Ring B is 2,3-dihydrobenzo[b][1,4]dioxinyl.

E35 The compound of embodiment E28, or a pharmaceutically acceptable salt thereof, wherein Ring B is pyrazolyl.

E36 The compound of embodiment E28, or a pharmaceutically acceptable salt thereof, wherein Ring B is pyrimidinyl.

E37 The compound of embodiment E28, or a pharmaceutically acceptable salt thereof, wherein Ring B is quinolinyl.

E38 The compound of embodiment E28, or a pharmaceutically acceptable salt thereof, wherein Ring B is indazolyl.

E39 A compound of Formula (III):

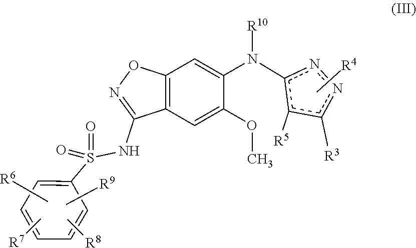

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl, wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E40 A compound of Formula (IIIa):

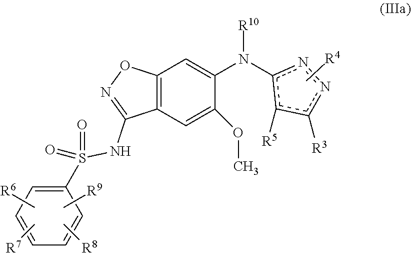

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E41 A compound of Formula (IIIb):

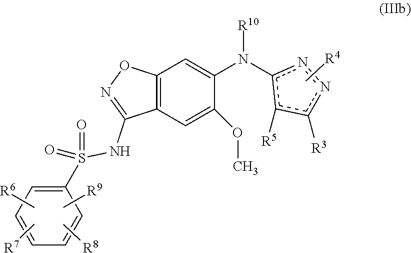

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by ethoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R⁹ is hydrogen or fluoro;
R¹⁰ is hydrogen or C₁₋₃ alkyl;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E42 The compound of any one of embodiments E1 to E15 and E17 to E41, or a pharmaceutically acceptable salt thereof, wherein R⁶ is methoxy, R⁷ is methoxy, R⁸ is hydrogen, and R⁹ is hydrogen.

E43 A compound of Formula (IV):

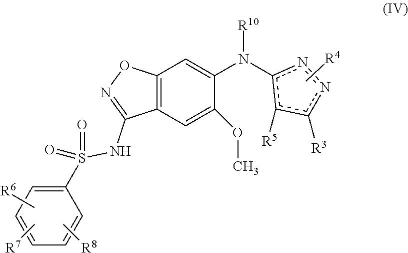

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E44 A compound of Formula (V):

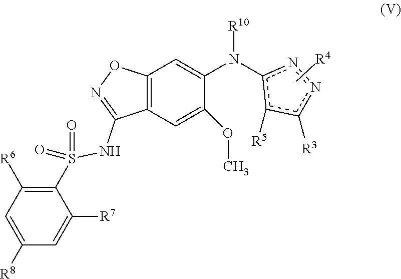

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E45 A compound of Formula (VI):

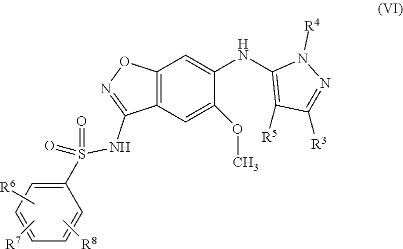

or a pharmaceutically acceptable salt thereof, wherein:

R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E46 A compound of Formula (VIa):

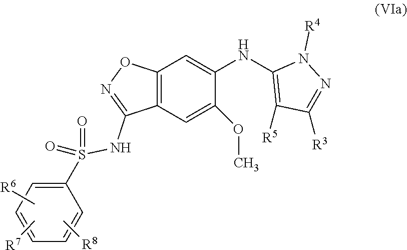

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E47 A compound of Formula (VIb):

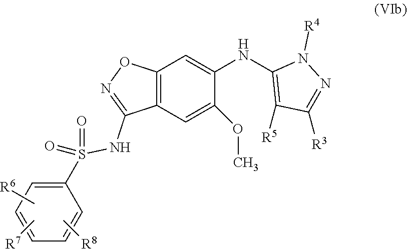

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E48 A compound of Formula (VII):

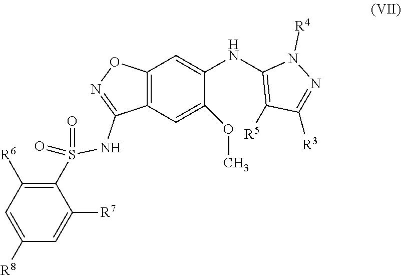

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E49 A compound of Formula (VIIa):

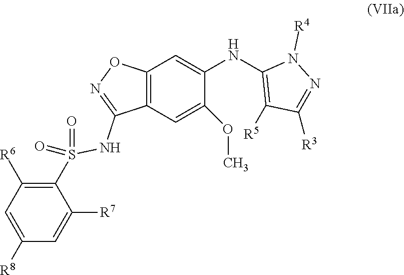

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E50 A compound of Formula (VIIb):

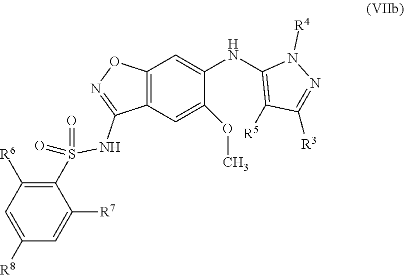

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E51 A compound of Formula (VIII):

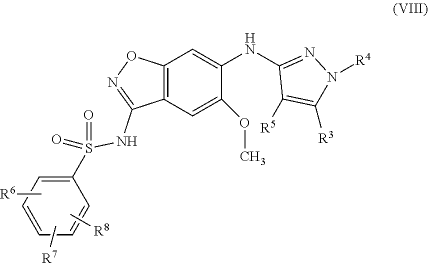

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E52 A compound of Formula (Villa):

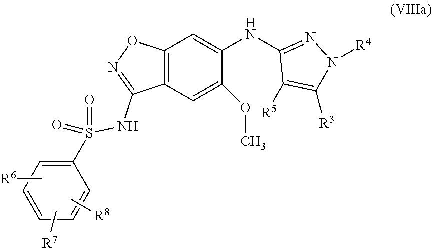

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E53 A compound of Formula (VIIIb):

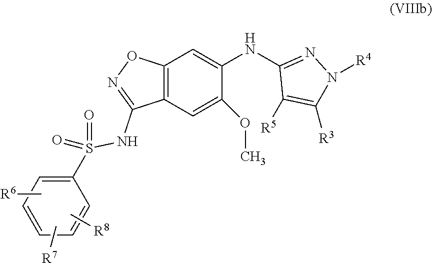

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E54 A compound of Formula (IX):

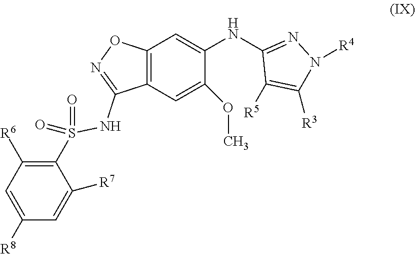

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₋₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1.

E55 A compound of Formula (IXa):

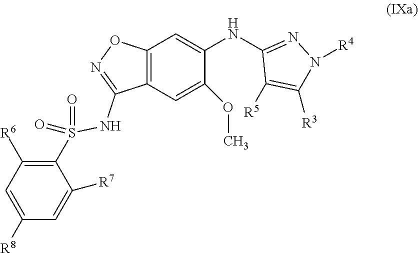

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CH₂)ₙ-4-6 membered heterocycloalkyl, 5-6 membered heteroaryl, —C(O)NR¹¹R¹², or —O-phenyl, wherein the C₁-C₄ alkyl is optionally substituted by methoxy, —N(R¹³)(R¹⁴), or one, two, or three fluorine atoms, wherein the phenyl is optionally substituted by methoxy, wherein the —(CH₂)ₙ-4-6 membered heterocycloalkyl and the 5-6 membered heteroaryl are each independently optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃;
R¹² is hydrogen or methyl;
each R¹³ and R¹⁴ are independently methyl or ethyl; and
n is 0 or 1.

E56 A compound of Formula (IXb):

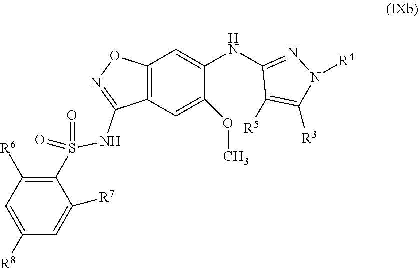

or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₅ cycloalkyl is optionally substituted by methyl or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, 4-6 membered heterocycloalkyl, 5-6 membered heteroaryl, —C(O)NR¹¹R¹², or —O-phenyl, wherein the C₁-C₄ alkyl is optionally substituted by methoxy or one, two, or three fluorine atoms, wherein the phenyl is optionally substituted by methoxy, wherein the 5-6 membered heteroaryl is optionally substituted by methyl, and wherein the —O-phenyl is optionally substituted by fluoro, methyl, or methoxy;
R¹¹ is hydrogen, methyl, —(CH₂)₅NH₂, or —(CH₂)₅NHC(O)-phenyl-(1,2,4,5-tetrazine)-CH₃; and
R¹² is hydrogen or methyl.

E57 The compound of any one of embodiments E1 to E56, or a pharmaceutically acceptable salt thereof, wherein R³ is hydrogen, chloro, C₁-C₄ alkyl, —CHF₂, —CF₃, —CF₂CH₃, —CH₂—CF₃, —CH₂OCH₃, methoxy, —O—CHF₂, —C(O)OH, —C(O)OCH₃, bicyclo[1.1.1]pentan-1-yl, cyclopropyl, cyclobutyl, cyclopentyl, piperidinyl, or benzyl, wherein the C₁-C₄ alkyl is optionally substituted by —OH and the cyclopropyl is optionally substituted by methyl or two fluorine atoms.

E58 The compound of embodiment E57, or a pharmaceutically acceptable salt thereof, wherein R³ is cyclopropyl.

E59 The compound of any one of embodiments E1 to E58, or a pharmaceutically acceptable salt thereof, wherein R⁴ is hydrogen, methyl, ethyl, or isopropyl.

E60 The compound or embodiment E59, or a pharmaceutically acceptable salt thereof, wherein R⁴ is hydrogen.

E61 The compound of any one of embodiments E1 to E60, or a pharmaceutically acceptable salt thereof, wherein R⁵ is hydrogen, fluoro, cyano, methyl, ethyl, or —C(O)NH₂.

E62 The compound of embodiment E61, or a pharmaceutically acceptable salt thereof, wherein R⁵ is hydrogen.

E63 The compound of embodiment E62, or a pharmaceutically acceptable salt thereof, wherein R³ is cyclopropyl, R⁴ is hydrogen and R⁵ is hydrogen.

E64 The compound of any one of embodiments E1 to E15 and E17 to E62, or a pharmaceutically acceptable salt thereof, wherein R⁶ is hydrogen, bromo, chloro, fluoro, methyl, ethyl, methoxy, ethoxy, —O-isopropyl, —O—CH₂CHF₂, —O—CF₃, —O-cyclobutyl, or —O-cyclopropyl.

E65 The compound of embodiment E64, or a pharmaceutically acceptable salt thereof, wherein R⁶ is methoxy.

E66 The compound of any one of embodiments E1 to E15 and E17 to E56, or a pharmaceutically acceptable salt thereof, wherein R⁷ is hydrogen, methyl, ethyl, or methoxy.

E67 The compound of embodiment E66, or a pharmaceutically acceptable salt thereof, wherein R⁷ is methoxy.

E68 The compound of embodiment E66, or a pharmaceutically acceptable salt thereof, wherein R⁷ is hydrogen.

E69 The compound of any one of embodiments E1 to E15 and E17 to E68, or a pharmaceutically acceptable salt thereof, wherein R⁸ is hydrogen, chloro, fluoro, cyano, methyl, ethyl, propyl, —CHF₂, —CF₃, —CH₂OCH₃, methoxy, phenyl, azetidinyl, oxetanyl, tetrahydrofuranyl, pyrrolidinyl, tetrahydropyranyl, piperidinyl, morpholinyl, imidazolyl, pyrazolyl, oxazolyl, thiazolyl, pyridinyl, pyrazinyl, —C(O)N(CH₃)₂, —C(O)NH(CH₂)₅NH₂, —C(O)NH(CH₂)₅NHC(O)-phenyl-(1,2,4,5-tetrazine)-CH₃ or —O-phenyl, wherein the phenyl is optionally substituted by methoxy, wherein the pyrazolyl, the imidazolyl and the thiazolyl are each independently optionally substituted by methyl, and wherein the —O-phenyl is optionally substituted by methyl, fluoro or methoxy.

E70 The compound of any one of embodiments E1 to E69, or a pharmaceutically acceptable salt thereof, wherein R⁸ is hydrogen, chloro, fluoro, cyano, methyl, ethyl, propyl, —CHF₂, —CF₃, —CH₂OCH₃, methoxy, phenyl, oxetanyl, tetrahydrofuranyl, tetrahydropyranyl, imidazolyl, pyrazolyl, oxazolyl, thiazolyl, pyridinyl, pyrazinyl, —C(O)N(CH₃)₂, —C(O)NH(CH₂)₅NH₂, —C(O)NH(CH₂)₅NHC(O)-phenyl-(1,2,4,5-tetrazine)-CH₃ or —O-phenyl, wherein the phenyl is optionally substituted by methoxy, wherein the pyrazolyl, the imidazolyl and the thiazolyl are each independently optionally substituted by methyl, and wherein the —O-phenyl is optionally substituted by methyl, fluoro or methoxy.

E71 The compound of embodiment E69 or embodiment E70, or a pharmaceutically acceptable salt thereof, wherein R⁸ is hydrogen.

E72 A compound of Formula (X):

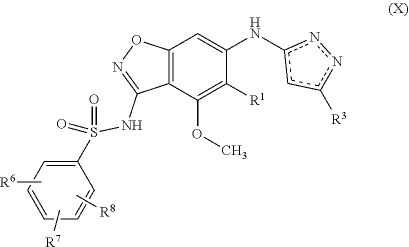

or a pharmaceutically acceptable salt thereof, wherein:
R¹ is hydrogen or fluoro;
R³ is hydrogen, ethyl or cyclopropyl;
R⁶ is methoxy, ethoxy, O-cyclopropyl, or O-cyclobutyl;
R⁷ is hydrogen, fluoro, or methoxy;
R⁸ is hydrogen, fluoro, methyl, or —CHF₂.

E73 The compound of any one of embodiments E1-E24 and E72, or a pharmaceutically acceptable salt thereof, wherein R¹ is fluoro and R² is methoxy.

E74 The compound of any one of embodiments E1-E24 and E72, or a pharmaceutically acceptable salt thereof, wherein R¹ is hydrogen and R² is methoxy.

E75 The compound of any one of embodiments E72 to E74, or a pharmaceutically acceptable salt thereof, wherein R³ is cyclopropyl.

E76 A compound selected from the group consisting of

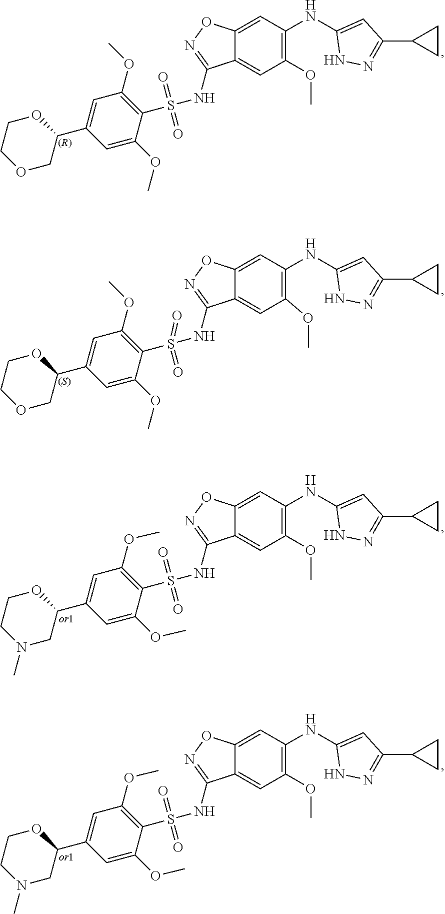

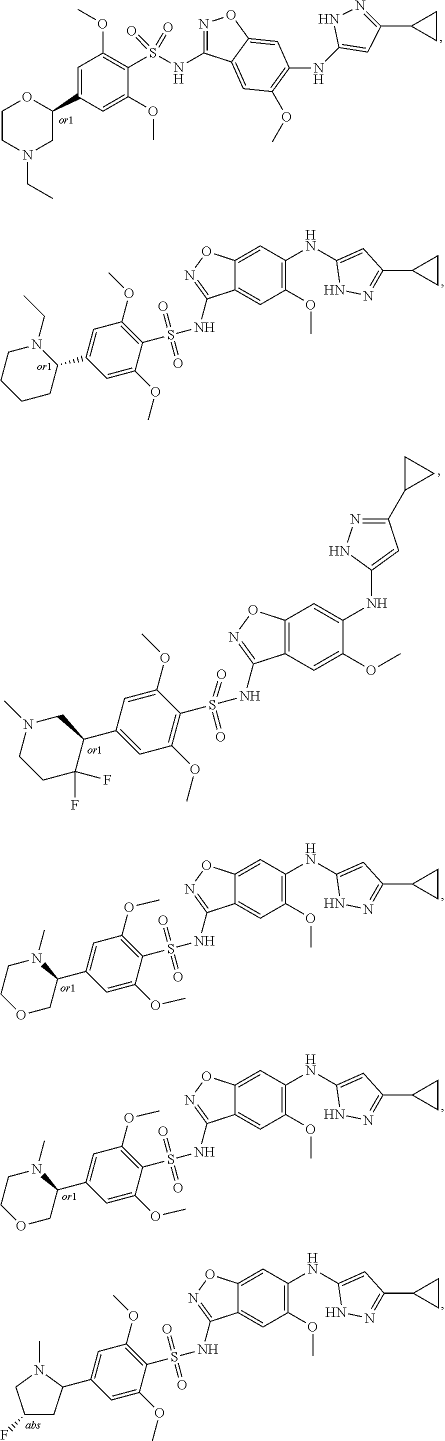

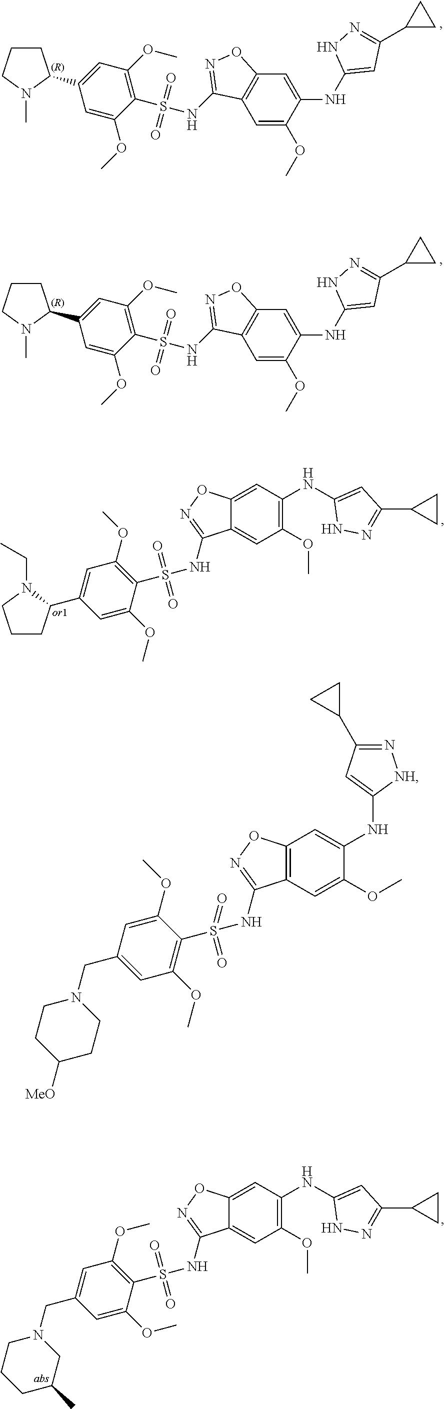

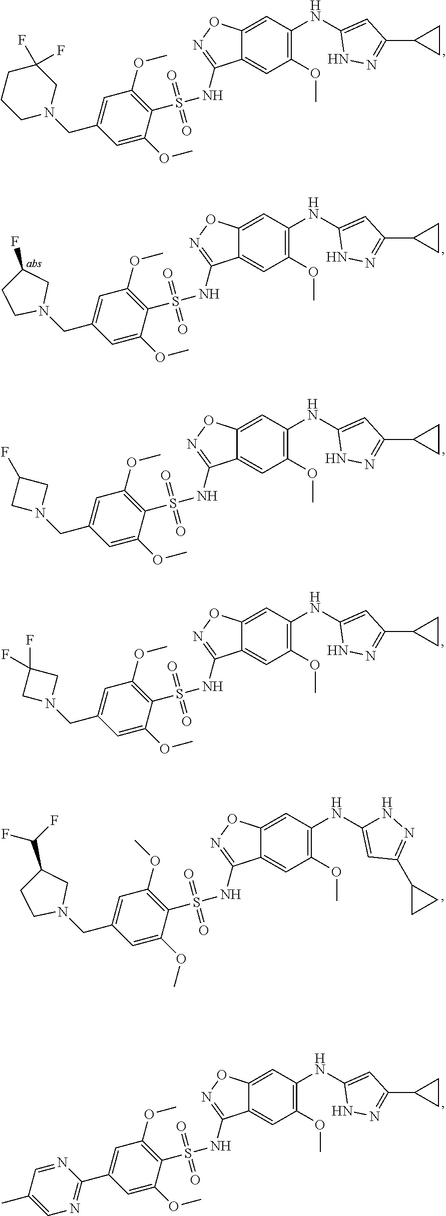 [SMILES: COc1cc2c(NS(=O)(=O)c3c(OC)cc(CN4CCCC(F)(F)C4)cc3OC)noc2cc1Nc1cc(C2CC2)n[nH]1 ; COc1cc2c(NS(=O)(=O)c3c(OC)cc(CN4CC[C@@H](F)C4)cc3OC)noc2cc1Nc1cc(C2CC2)n[nH]1 ; COc1cc2c(NS(=O)(=O)c3c(OC)cc(CN4CC(F)C4)cc3OC)noc2cc1Nc1cc(C2CC2)n[nH]1 ; COc1cc2c(NS(=O)(=O)c3c(OC)cc(CN4CC(F)(F)C4)cc3OC)noc2cc1Nc1cc(C2CC2)n[nH]1 ; COc1cc2c(NS(=O)(=O)c3c(OC)cc(CN4CC[C@@H](C(F)F)C4)cc3OC)noc2cc1Nc1cc(C2CC2)n[nH]1 ; COc1cc2c(NS(=O)(=O)c3c(OC)cc(-c4ncc(C)cn4)cc3OC)noc2cc1Nc1cc(C2CC2)n[nH]1]

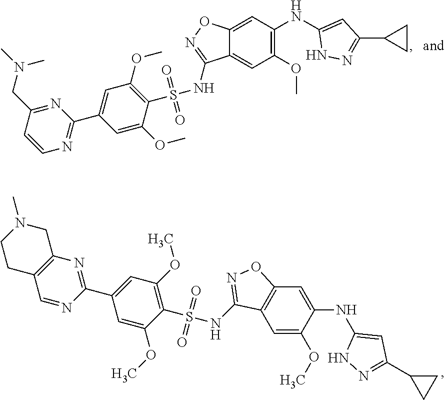 [SMILES: COc1cc2c(NS(=O)(=O)c3c(OC)cc(-c4nccc(CN(C)C)n4)cc3OC)noc2cc1Nc1cc(C2CC2)n[nH]1 ; COc1cc2c(NS(=O)(=O)c3c(OC)cc(-c4ncc5c(n4)CN(C)CC5)cc3OC)noc2cc1Nc1cc(C2CC2)n[nH]1]

or a tautomer thereof, or a pharmaceutically acceptable salt of the compound or the tautomer thereof.

E77 The compound of embodiment E76, which is

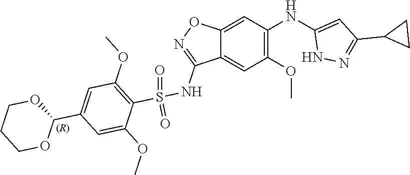

or a tautomer thereof, or a pharmaceutically acceptable salt of the compound or the tautomer thereof.

E78 The compound of embodiment E76, which is

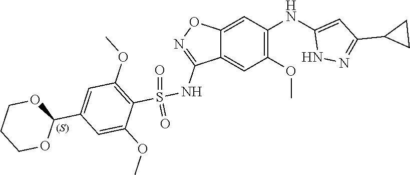

or a tautomer thereof, or a pharmaceutically acceptable salt of the compound or the tautomer thereof.

E79 The compound of embodiment E76, which is

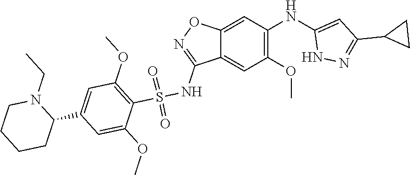 [SMILES: CCN1CCCC[C@H]1c1cc(OC)c(S(=O)(=O)Nc2noc3cc(Nc4cc(C5CC5)n[nH]4)c(OC)cc23)c(OC)c1]

or a tautomer thereof, or a pharmaceutically acceptable salt of the compound or the tautomer thereof.

E80 The compound of embodiment E76, which is

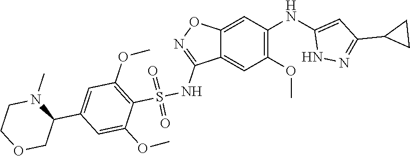 [SMILES: COc1cc2c(NS(=O)(=O)c3c(OC)cc([C@H]4COCCN4C)cc3OC)noc2cc1Nc1cc(C2CC2)n[nH]1]

or a tautomer thereof, or a pharmaceutically acceptable salt of the compound or the tautomer thereof.

E81 The compound of embodiment E76, which is

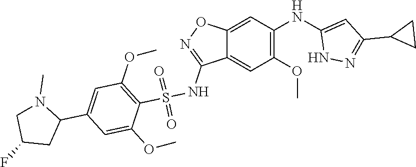 [SMILES: COc1cc2c(NS(=O)(=O)c3c(OC)cc(C4C[C@H](F)CN4C)cc3OC)noc2cc1Nc1cc(C2CC2)n[nH]1]

or a tautomer thereof, or a pharmaceutically acceptable salt of the compound or the tautomer thereof.

E82 The compound of embodiment E76, which is

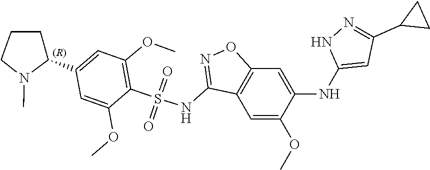

or a tautomer thereof, or a pharmaceutically acceptable salt of the compound or the tautomer thereof.

E83 The compound of embodiment E76, which is

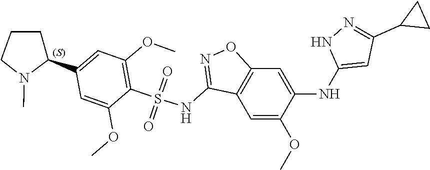

or a tautomer thereof, or a pharmaceutically acceptable salt of the compound or the tautomer thereof.

E84 The compound of embodiment E76, which is

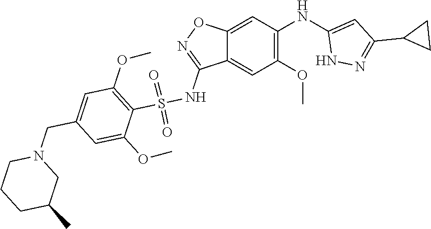 [SMILES: COc1cc2c(NS(=O)(=O)c3c(OC)cc(CN4CCC[C@H](C)C4)cc3OC)noc2cc1Nc1cc(C2CC2)n[nH]1]

or a tautomer thereof, or a pharmaceutically acceptable salt of the compound or the tautomer thereof.

E85 The compound of embodiment E76, which is

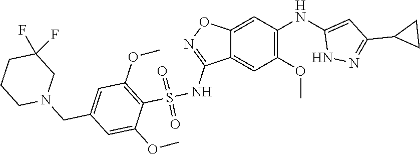 [SMILES: COc1cc2c(NS(=O)(=O)c3c(OC)cc(CN4CCCC(F)(F)C4)cc3OC)noc2cc1Nc1cc(C2CC2)n[nH]1]

or a tautomer thereof, or a pharmaceutically acceptable salt of the compound or the tautomer thereof.

E86 The compound of embodiment E76, which is

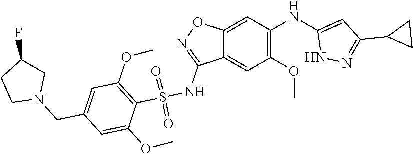 [SMILES: COc1cc2c(NS(=O)(=O)c3c(OC)cc(CN4CC[C@@H](F)C4)cc3OC)noc2cc1Nc1cc(C2CC2)n[nH]1]

or a tautomer thereof, or a pharmaceutically acceptable salt of the compound or the tautomer thereof.

E87 The compound of embodiment E76, which is

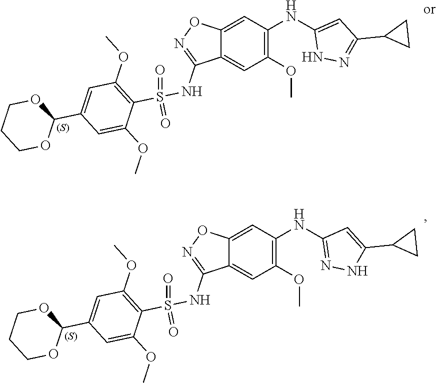

or a pharmaceutically acceptable salt thereof.

E88 The compound of embodiment E76, which is

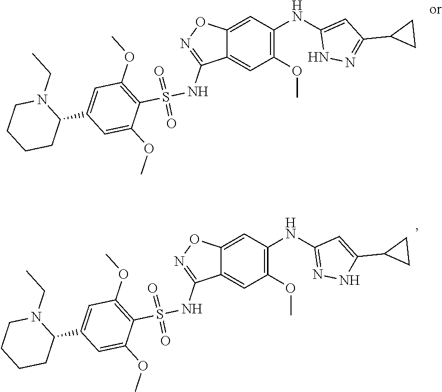 [SMILES: CCN1CCCC[C@H]1c1cc(OC)c(S(=O)(=O)Nc2noc3cc(Nc4cc(C5CC5)n[nH]4)c(OC)cc23)c(OC)c1 ; CCN1CCCC[C@H]1c1cc(OC)c(S(=O)(=O)Nc2noc3cc(Nc4cc(C5CC5)[nH]n4)c(OC)cc23)c(OC)c1]

or a pharmaceutically acceptable salt thereof.

E89 The compound of embodiment E76, which is

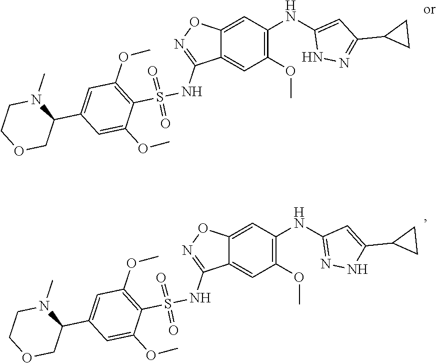 [SMILES: COc1cc2c(NS(=O)(=O)c3c(OC)cc([C@H]4COCCN4C)cc3OC)noc2cc1Nc1cc(C2CC2)n[nH]1 ; COc1cc2c(NS(=O)(=O)c3c(OC)cc([C@H]4COCCN4C)cc3OC)noc2cc1Nc1cc(C2CC2)[nH]n1]

or a pharmaceutically acceptable salt thereof.

E90 The compound of embodiment E76, which is

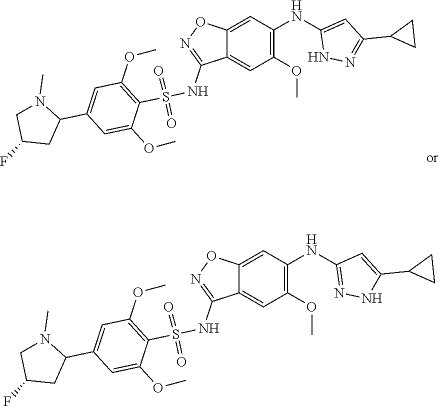 [SMILES: COc1cc2c(NS(=O)(=O)c3c(OC)cc(C4C[C@H](F)CN4C)cc3OC)noc2cc1Nc1cc(C2CC2)n[nH]1 ; COc1cc2c(NS(=O)(=O)c3c(OC)cc(C4C[C@H](F)CN4C)cc3OC)noc2cc1Nc1cc(C2CC2)[nH]n1]

or a pharmaceutically acceptable salt thereof.

E91 The compound of embodiment E76, which is

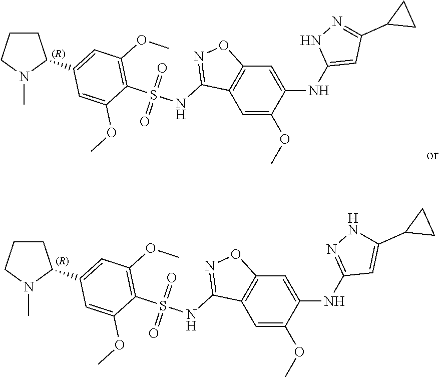

or a pharmaceutically acceptable salt thereof.

E92 The compound of embodiment E76, which is

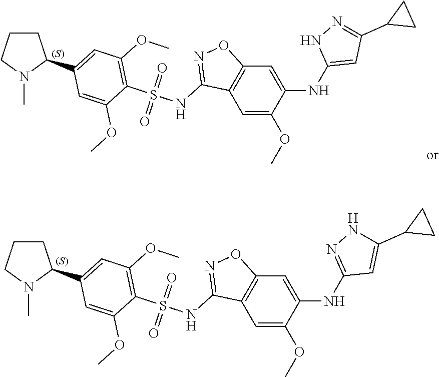

or a pharmaceutically acceptable salt thereof.

E93 The compound of embodiment E76, which is

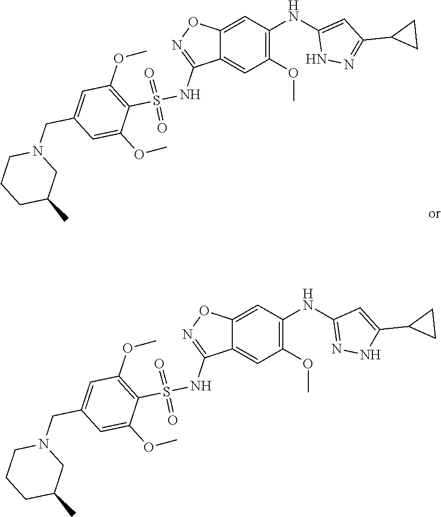 [SMILES: COc1cc2c(NS(=O)(=O)c3c(OC)cc(CN4CCC[C@H](C)C4)cc3OC)noc2cc1Nc1cc(C2CC2)n[nH]1 ; COc1cc2c(NS(=O)(=O)c3c(OC)cc(CN4CCC[C@H](C)C4)cc3OC)noc2cc1Nc1cc(C2CC2)[nH]n1]

or a pharmaceutically acceptable salt thereof.

E94 The compound of embodiment E76, which is

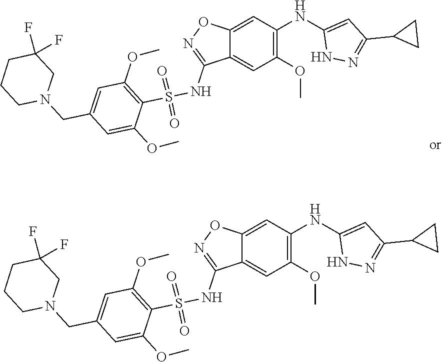 [SMILES: COc1cc2c(NS(=O)(=O)c3c(OC)cc(CN4CCCC(F)(F)C4)cc3OC)noc2cc1Nc1cc(C2CC2)n[nH]1 ; COc1cc2c(NS(=O)(=O)c3c(OC)cc(CN4CCCC(F)(F)C4)cc3OC)noc2cc1Nc1cc(C2CC2)n[nH]1]

or a pharmaceutically acceptable salt thereof.

E95 The compound of embodiment E76, which is

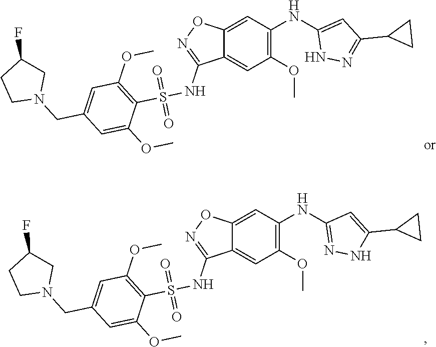 [SMILES: COc1cc2c(NS(=O)(=O)c3c(OC)cc(CN4CC[C@@H](F)C4)cc3OC)noc2cc1Nc1cc(C2CC2)n[nH]1 ; COc1cc2c(NS(=O)(=O)c3c(OC)cc(CN4CC[C@@H](F)C4)cc3OC)noc2cc1Nc1cc(C2CC2)[nH]n1]

or a pharmaceutically acceptable salt thereof.

E96 A pharmaceutical composition comprising the compound according to any of embodiments E1 to E94, or a pharmaceutically acceptable salt thereof, and at least one pharmaceutically acceptable excipient.

E97 A method for treating cancer, comprising administering to a subject in need thereof a therapeutically effective amount of the compound of any one of embodiments E1 to E95, or a pharmaceutically acceptable salt thereof.

E98 The method of embodiment E96, wherein the cancer is breast cancer.

E99 The method of embodiment E97, wherein the breast cancer is ER+ breast cancer.

E100 The method of embodiment E98, wherein the ER+ breast cancer is ER+ HER2− breast cancer.

E101 A method for treating cancer, comprising administering to a subject in need thereof a therapeutically effective amount of the compound of any one of embodiments E1 to E99, or a pharmaceutically acceptable salt thereof, and further comprising administering an amount of an additional therapeutic agent.

E102 The method of embodiment E100, wherein the cancer is breast cancer.

E103 The method of embodiment E101, wherein the breast cancer is ER+ breast cancer.

E104 The method of embodiment E102, wherein the ER+ breast cancer is ER+ HER2− breast cancer.

E105 A compound of any one of embodiments E1 to E103, or a pharmaceutically acceptable salt thereof, for use as a medicament.

E106 A compound of any one of embodiments E1 to E104, or a pharmaceutically acceptable salt thereof, for use in the treatment of cancer.

E107 Use of a compound of any one of embodiments E1 to E105, or a pharmaceutically acceptable salt thereof, for the manufacture of a medicament for the treatment of cancer.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein.

Furthermore, each of the embodiments described herein envisions within its scope pharmaceutically acceptable salts of the compounds described herein.

### Definitions

Unless otherwise defined herein, scientific and technical terms used in connection with the present invention have the meanings that are commonly understood by those of ordinary skill in the art.

The invention described herein suitably may be practiced in the absence of any element(s) not specifically disclosed herein.

As used herein, the singular form “a”, “an”, and “the” include plural references unless indicated otherwise. For example, “a” substituent includes one or more substituents.

As used herein, the term “about” when used to modify a numerically defined parameter (e.g., the dose of 5 mg) means that the parameter may vary by as much as 10% below or above the stated numerical value for that parameter. For example, a dose of about 5 mg means 5 mg±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The term “optionally substituted” is used to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, and the like. In an embodiment, the alkyl is methyl, ethyl, n-propyl, isopropyl, n-butyl, sec-butyl, isobutyl, or tert-butyl. Alkyl groups may be optionally substituted, unsubstituted or substituted, as further defined herein.

“Cycloalkyl” refers to a fully saturated hydrocarbon ring system that has the specified number of carbon atoms, which may be a monocyclic or bridged ring system that is connected to the base molecule through a carbon atom of the cycloalkyl ring. Cycloalkyl groups may contain, but are not limited to, 3 to 6 carbon atoms (“C₃-C₆ cycloalkyl”), 3 to 5 carbon atoms (“C₃-C₅ cycloalkyl”), 4 to 5 carbon atoms (“C₄-C₅ cycloalkyl”) or 3 to 4 carbon atoms (“C₃-C₄ cycloalkyl”). Examples include, but are not limited to, cyclopropyl, cyclobutyl, cyclopentyl, bicyclo[1.1.1]pentan-1-yl, cyclohexyl, and the like. In an embodiment, the cycloalkyl group is cyclopropyl, cyclobutyl, cyclopentyl, or bicyclo[1.1.1]pentan-1-yl. Cycloalkyl groups may be optionally substituted, unsubstituted or substituted, as further defined herein.

Heterocycloalkyl” refers to a fully saturated ring system containing the specified number of ring atoms and containing at least one heteroatom selected from nitrogen and oxygen as a ring member, and where the heterocycloalkyl ring is connected to the base molecule via a ring atom, which may be C or N. Heterocycloalkyl rings may contain 1 to 2 heteroatoms selected from N and O as ring members, provided that such heterocycloalkyl rings do not contain two contiguous nitrogen or oxygen atoms. Heterocycloalkyl rings include rings which are spirocyclic, where such spirocyclic ring is saturated, provided the point of attachment to the base molecule is an atom of the heterocycloalkyl portion of the ring system. “4-8 membered heterocycloalkyl” contain from four to eight ring atoms, “4-6 membered heterocycloalkyl” contain from four to six ring atoms, “6 membered heterocycloalkyl” contain from four to six ring atoms, and “4-5 membered heterocycloalkyl” contain from four to five ring atoms. Heterocycloalkyl rings may be optionally substituted, unsubstituted or substituted, as further defined herein. Examples of heterocycloalkyl groups include, but are not limited to:

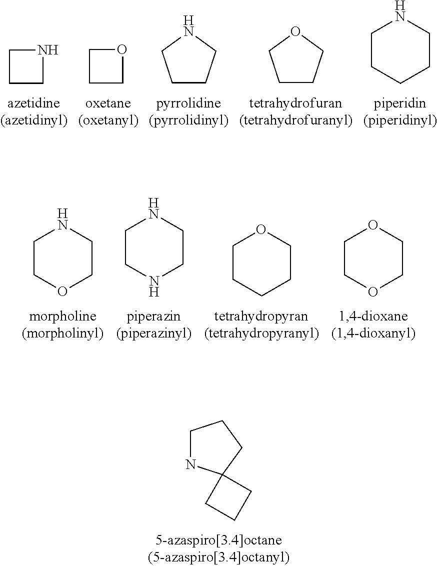

“Aryl” or “aromatic” refers to monocyclic or bicyclic (e.g., biaryl, fused) ring systems that contain the specified number of ring atoms, in which all carbon atoms in the ring are of sp² hybridization and in which the pi electrons are in conjugation. Aryl groups may contain, but are not limited to, 6 to 10 carbon atoms (“C₆-C₁₀ aryl”). Fused aryl groups may include an aryl ring (e.g., a phenyl ring) fused to another aryl ring. Examples include, but are not limited to, phenyl and naphthyl. Also included within the scope of the term “aryl”, as it is used herein, is a group in which an aromatic ring is fused to one or more non-aromatic rings, such as indanyl (2,3-dihydro-1H-indene) and tetrahydronaphthyl (also known as 1,2,3,4-tetrahydronaphthyl), where the radical or point of attachment is on the aromatic ring. Aryl groups may be optionally substituted, unsubstituted or substituted, as further defined herein.

“Heteroaryl” or “heteroaromatic” refers to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl rings include rings which are spirocyclic, bridged, or fused to one or more other cycloalkyl or heterocycloalkyl rings, where such spirocyclic, bridged, or fused rings may themselves be saturated, partially unsaturated or aromatic to the extent unsaturation or aromaticity makes chemical sense, provided the point of attachment to the base molecule is an atom of the aromatic portion of the ring system.

Heteroaryl groups may contain, but are not limited to, 5 to 10 ring atoms (“5-10 membered heteroaryl”), 5 to 8 ring atoms (“5-8 membered heteroaryl”), 9 to 10 ring atoms (“9-10 membered heteroaryl”), or 5 to 6 ring atoms (“5-6 membered heteroaryl”). Heteroaryl rings are attached to the base molecule via a ring atom of the aromatic ring. Thus, either 5- or 6-membered heteroaryl rings, alone or in fused or polycyclic ring structures, may be attached to the base molecule via a ring C or N atom. Also included within the scope of the term “heteroaryl”, as defined herein, is a 5- or 6-membered monocyclic heteroaryl ring that may be fused to a cycloalkyl or heterocycloalkyl to form a fused or polycyclic ring structure. Examples of heteroaryl groups, as defined herein include, but are not limited to, pyrrolyl (1H-pyrrolyl), pyrazolyl (1H-pyrazolyl), imidazolyl (1H-imidazolyl), isoxazolyl, oxazolyl, oxadiazolyl (1-oxa-2,4-diazolyl), thiazolyl, triazolyl (1H-1,2,3-triazole, 1H-1,2,4-triazole), pyridinyl, pyridazinyl, pyrimidinyl, pyrazinyl, indazolyl (2H-indazolyl), quinolinyl, quinoxalinyl, chromanyl, isochromanyl, 2,3-dihydrobenzo[b][1,4]dioxinyl, 5,6-dihydro-4H-pyrrolo[1,2-b]pyrazolyl, 5′,6′-dihydrospiro[cyclopropane-1,4′-pyrrolo[1,2-b]pyrazolyl] or tetrahydropyrrolo[3,4-c]pyrazolyl, hexahydropyrido[3,4-d]pyrimidinyl. In a preferred embodiment, the heteroaryl is pyrazolyl. Heteroaryl groups may be optionally substituted, unsubstituted or substituted, as further defined herein.

Illustrative examples of monocyclic heteroaryl groups include, but are not limited to a monovalent radical of:

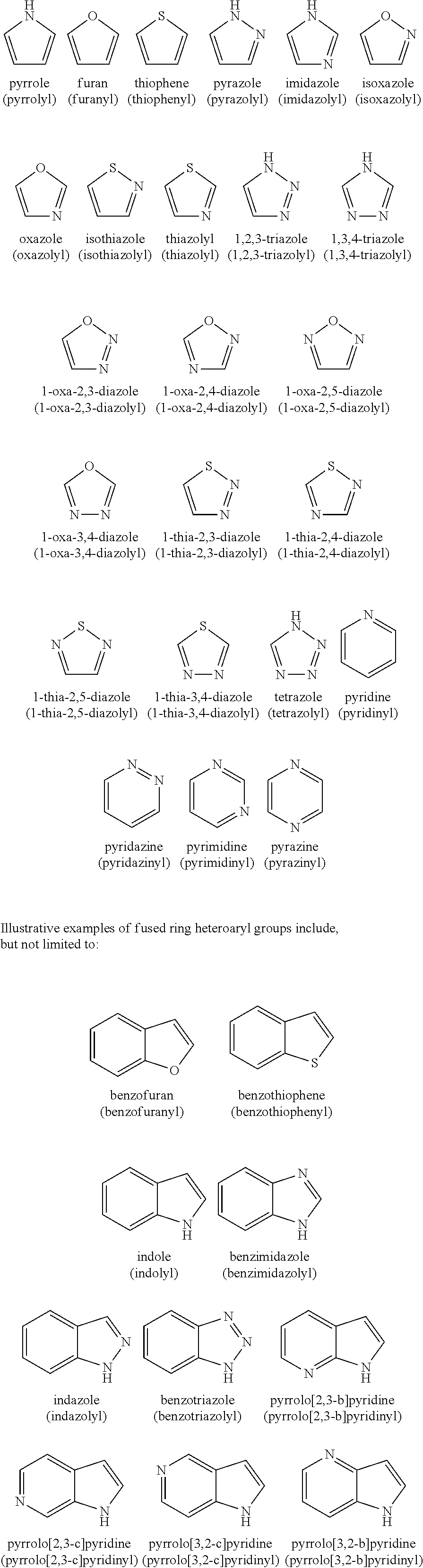

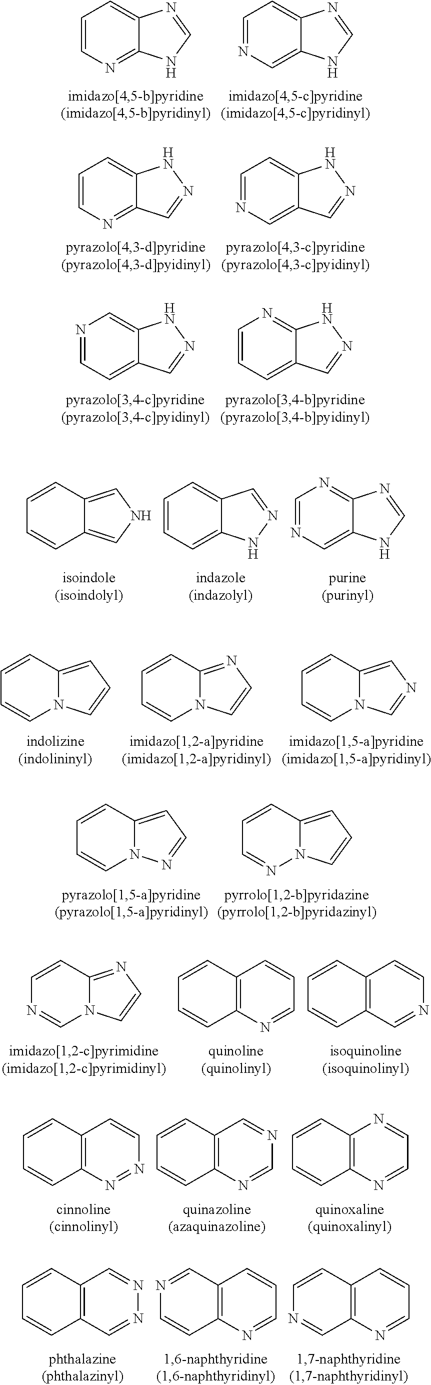

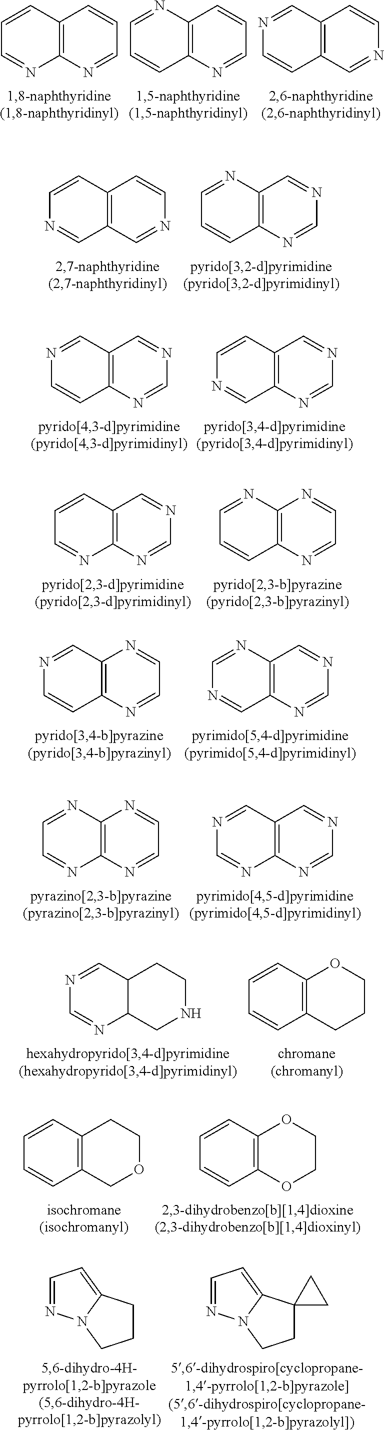

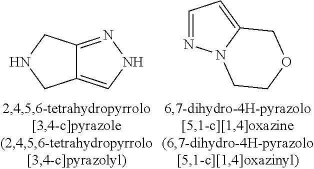

The term “pharmaceutically acceptable” means the substance (e.g., the compounds described herein) and any salt thereof, or composition containing the substance or salt of the invention is suitable for administration to a subject or patient.

“Compounds of the invention” include compounds of Formulas (I)-(X), (Ia)-(VIIa), (Ib)-(VIIb) and the Examples used in the preparation thereof. One of ordinary skill in the art will appreciate that compounds of the invention, and novel intermediates thereof, include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the invention, and novel intermediates thereof, include solvates, hydrates, isomorphs, polymorphs, esters, salt forms, prodrugs, and isotopically labelled versions thereof (including deuterium substitutions), where they may be formed.

Compounds of the inventions act as KAT6/7 inhibitors that are selective against KAT5 and KAT8. The MYST family of histone lysine acetyltransferases, KAT5, KAT6A, KAT6B, KAT7, and KAT8, regulate gene transcription important for cellular functions. This class of enzymes share homologous MYST domains, but exist in unique complexes that dictate their function. The combined inhibition of KAT6A/B and KAT7 drive selective dependencies in cancer cells, as predicted by the Cancer Dependency Map (DepMap) (https://depmap.org/portal) through loss of histone acetylation at H3K23, H3K14 and H4K12. Conversely, KAT5 and KAT8 are classified as essential genes in the DepMap, and knockout mouse models demonstrate embryonic lethality (Thomas, Tim, et al. “Mof (MYST1 or KAT8) is essential for progression of embryonic development past the blastocyst stage and required for normal chromatin architecture.” Molecular and cellular biology 28.16 (2008): 5093-5105; Hu, Yaofei, et al. “Homozygous disruption of the Tip60 gene causes early embryonic lethality.” Developmental dynamics: an official publication of the American Association of Anatomists 238.11 (2009): 2912-2921). KAT8, as the catalytic component of the NSL complex, is required for cell survival and regulates transcription of essential genes (Radzisheuskaya A, Shliaha P V, Grinev V V, Shlyueva D, Damhofer H, Koche R, Gorshkov V, Kovalchuk S, Zhan Y, Rodriguez K L, Johnstone A L, Keogh M C, Hendrickson R C, Jensen O N, Helin K. Complex-dependent histone acetyltransferase activity of KAT8 determines its role in transcription and cellular homeostasis. Mol Cell. 2021 Apr. 15; 81 (8):1749-1765.e8. doi: 10.1016/j.molcel.2021.02.012. Epub 2021 Mar. 2. PMID: 33657400; PMCID: PMC8056186)

Compounds of the invention include, but are not limited to:

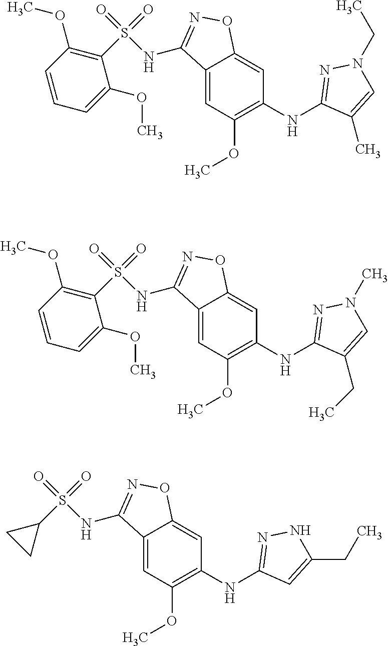

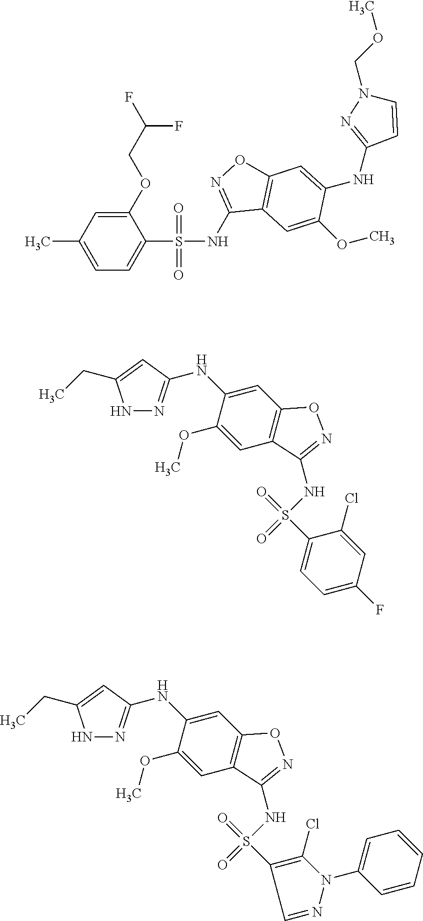 [SMILES: COCn1ccc(Nc2cc3onc(NS(=O)(=O)c4ccc(C)cc4OCC(F)F)c3cc2OC)n1 ; CCc1cc(Nc2cc3onc(NS(=O)(=O)c4ccc(F)cc4Cl)c3cc2OC)n[nH]1 ; CCc1cc(Nc2cc3onc(NS(=O)(=O)c4cnn(-c5ccccc5)c4Cl)c3cc2OC)n[nH]1]

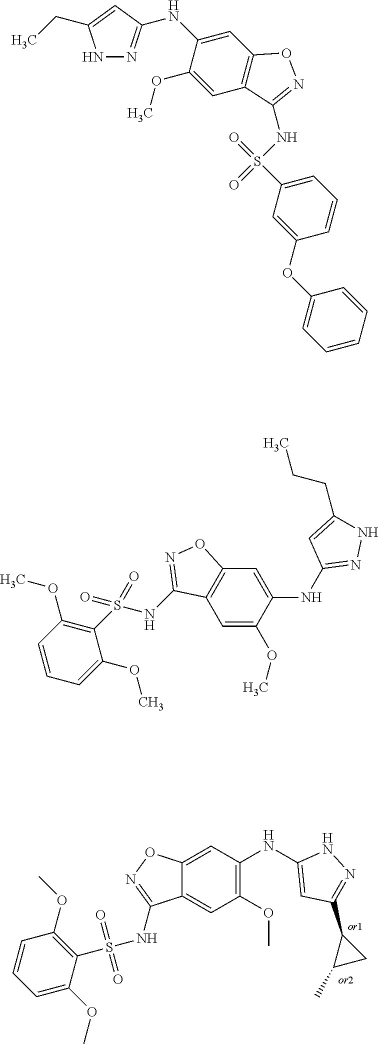 [SMILES: CCc1cc(Nc2cc3onc(NS(=O)(=O)c4cccc(Oc5ccccc5)c4)c3cc2OC)n[nH]1 ; CCCc1cc(Nc2cc3onc(NS(=O)(=O)c4c(OC)cccc4OC)c3cc2OC)n[nH]1 ; COc1cc2c(NS(=O)(=O)c3c(OC)cccc3OC)noc2cc1Nc1cc([C@H]2C[C@@H]2C)n[nH]1]

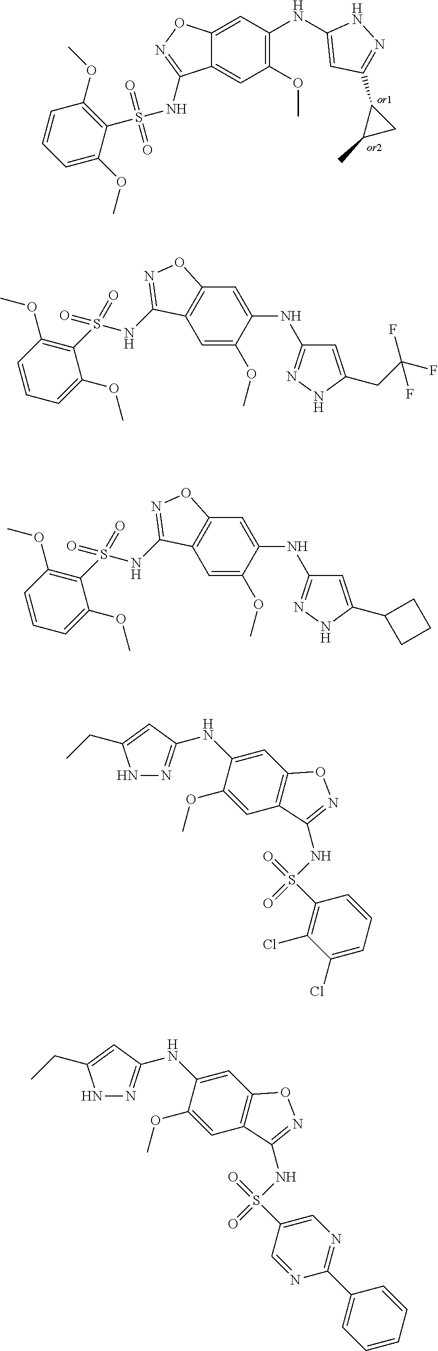 [SMILES: COc1cc2c(NS(=O)(=O)c3c(OC)cccc3OC)noc2cc1Nc1cc([C@@H]2C[C@H]2C)n[nH]1 ; COc1cc2c(NS(=O)(=O)c3c(OC)cccc3OC)noc2cc1Nc1cc(CC(F)(F)F)[nH]n1 ; COc1cc2c(NS(=O)(=O)c3c(OC)cccc3OC)noc2cc1Nc1cc(C2CCC2)[nH]n1 ; CCc1cc(Nc2cc3onc(NS(=O)(=O)c4cccc(Cl)c4Cl)c3cc2OC)n[nH]1 ; CCc1cc(Nc2cc3onc(NS(=O)(=O)c4cnc(-c5ccccc5)nc4)c3cc2OC)n[nH]1]

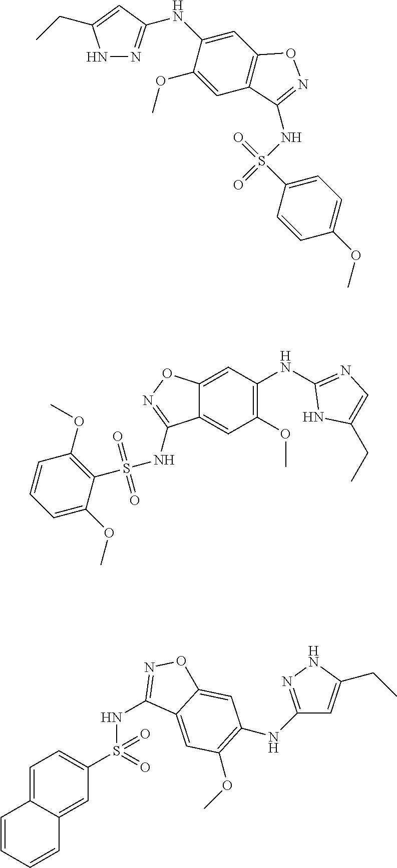 [SMILES: CCc1cc(Nc2cc3onc(NS(=O)(=O)c4ccc(OC)cc4)c3cc2OC)n[nH]1 ; CCc1cnc(Nc2cc3onc(NS(=O)(=O)c4c(OC)cccc4OC)c3cc2OC)[nH]1 ; CCc1cc(Nc2cc3onc(NS(=O)(=O)c4ccc5ccccc5c4)c3cc2OC)n[nH]1]

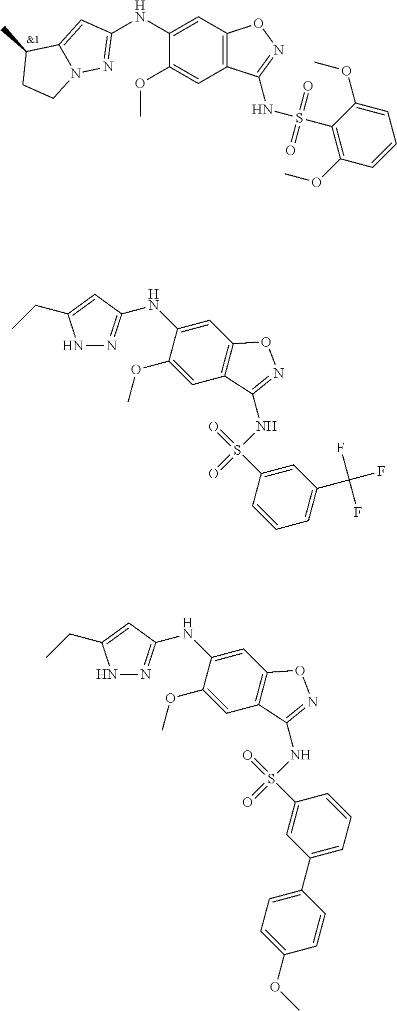 [SMILES: COc1cc2c(NS(=O)(=O)c3c(OC)cccc3OC)noc2cc1Nc1cc2n(n1)CC[C@H]2C ; CCc1cc(Nc2cc3onc(NS(=O)(=O)c4cccc(C(F)(F)F)c4)c3cc2OC)n[nH]1 ; CCc1cc(Nc2cc3onc(NS(=O)(=O)c4cccc(-c5ccc(OC)cc5)c4)c3cc2OC)n[nH]1]

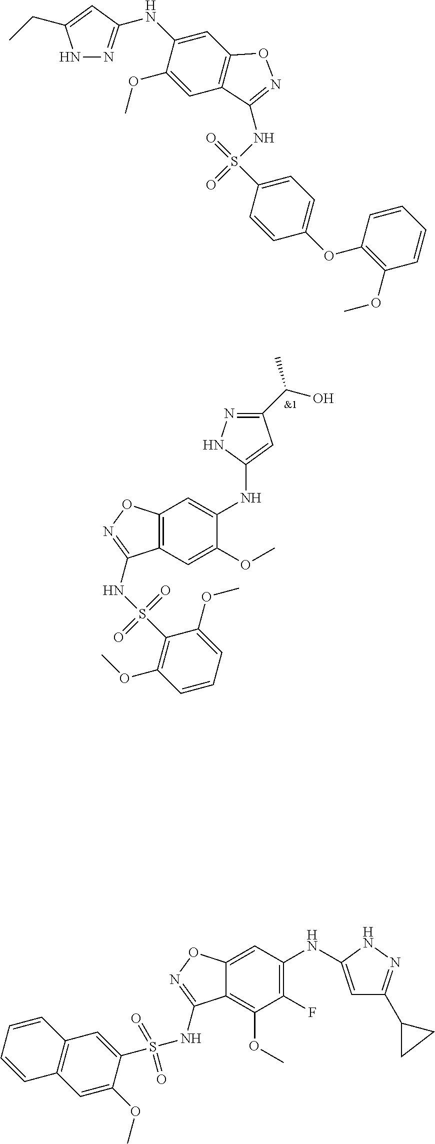 [SMILES: CCc1cc(Nc2cc3onc(NS(=O)(=O)c4ccc(Oc5ccccc5OC)cc4)c3cc2OC)n[nH]1 ; COc1cc2c(NS(=O)(=O)c3c(OC)cccc3OC)noc2cc1Nc1cc([C@H](C)O)n[nH]1 ; COc1cc2ccccc2cc1S(=O)(=O)Nc1noc2cc(Nc3cc(C4CC4)n[nH]3)c(F)c(OC)c12]

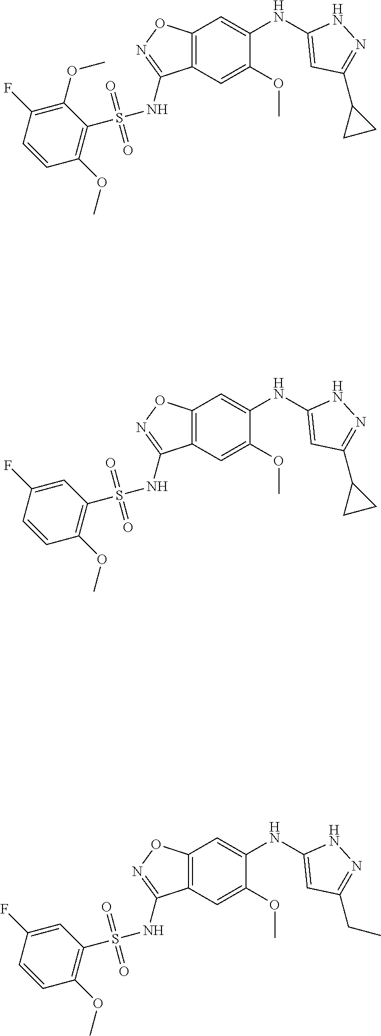 [SMILES: COc1cc2c(NS(=O)(=O)c3c(OC)ccc(F)c3OC)noc2cc1Nc1cc(C2CC2)n[nH]1 ; COc1cc2c(NS(=O)(=O)c3cc(F)ccc3OC)noc2cc1Nc1cc(C2CC2)n[nH]1 ; CCc1cc(Nc2cc3onc(NS(=O)(=O)c4cc(F)ccc4OC)c3cc2OC)[nH]n1]

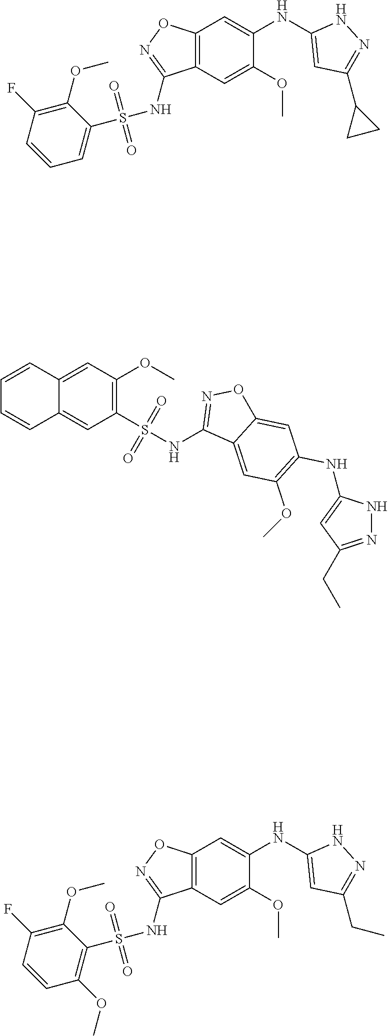 [SMILES: COc1cc2c(NS(=O)(=O)c3cccc(F)c3OC)noc2cc1Nc1cc(C2CC2)n[nH]1 ; CCc1cc(Nc2cc3onc(NS(=O)(=O)c4cc5ccccc5cc4OC)c3cc2OC)[nH]n1 ; CCc1cc(Nc2cc3onc(NS(=O)(=O)c4c(OC)ccc(F)c4OC)c3cc2OC)[nH]n1]

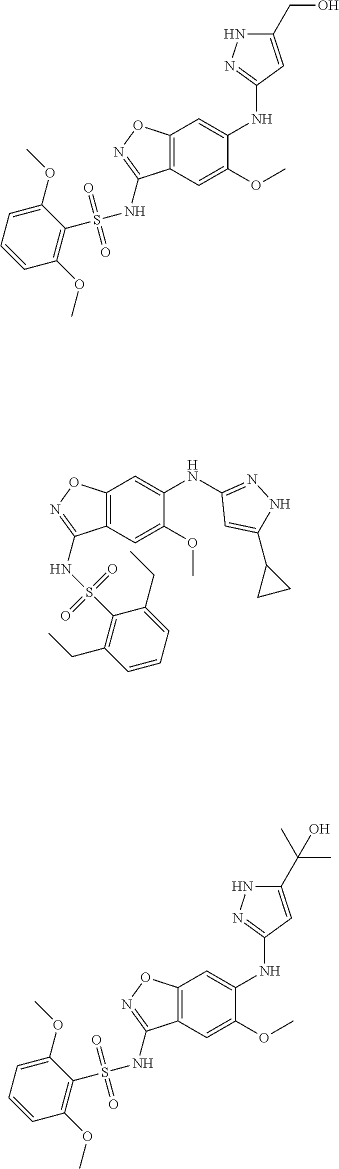 [SMILES: COc1cc2c(NS(=O)(=O)c3c(OC)cccc3OC)noc2cc1Nc1cc(CO)[nH]n1 ; CCc1cccc(CC)c1S(=O)(=O)Nc1noc2cc(Nc3cc(C4CC4)[nH]n3)c(OC)cc12 ; COc1cc2c(NS(=O)(=O)c3c(OC)cccc3OC)noc2cc1Nc1cc(C(C)(C)O)[nH]n1]

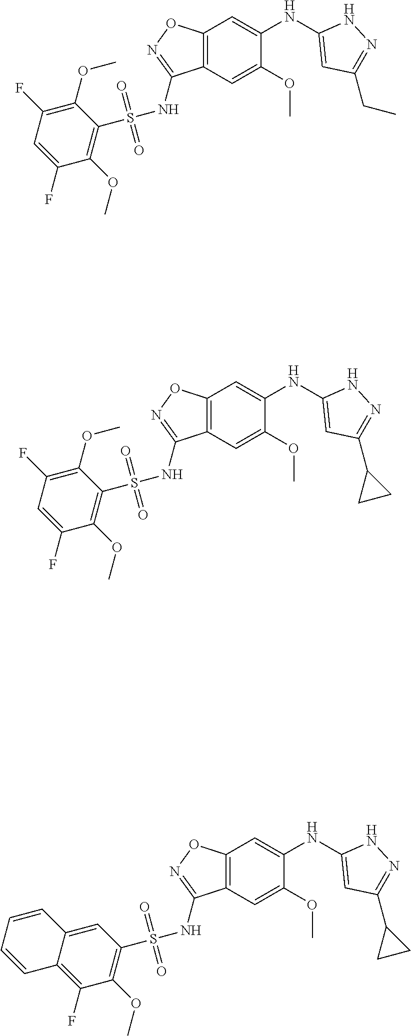 [SMILES: CCc1cc(Nc2cc3onc(NS(=O)(=O)c4c(OC)c(F)cc(F)c4OC)c3cc2OC)[nH]n1 ; COc1cc2c(NS(=O)(=O)c3c(OC)c(F)cc(F)c3OC)noc2cc1Nc1cc(C2CC2)n[nH]1 ; COc1cc2c(NS(=O)(=O)c3cc4ccccc4c(F)c3OC)noc2cc1Nc1cc(C2CC2)n[nH]1]

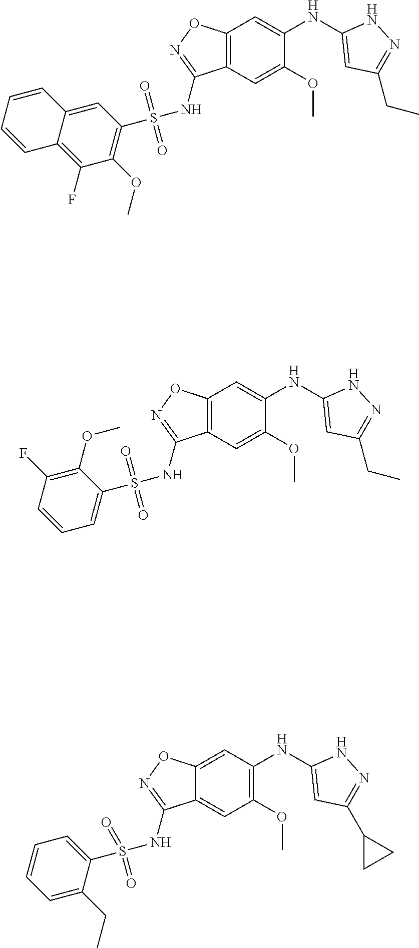 [SMILES: CCc1cc(Nc2cc3onc(NS(=O)(=O)c4cc5ccccc5c(F)c4OC)c3cc2OC)[nH]n1 ; CCc1cc(Nc2cc3onc(NS(=O)(=O)c4cccc(F)c4OC)c3cc2OC)[nH]n1 ; CCc1ccccc1S(=O)(=O)Nc1noc2cc(Nc3cc(C4CC4)n[nH]3)c(OC)cc12]

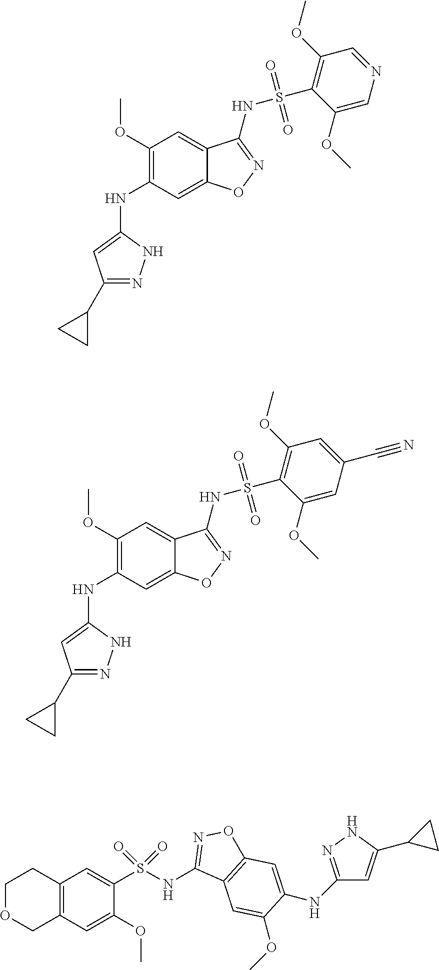 [SMILES: COc1cc2c(NS(=O)(=O)c3c(OC)cncc3OC)noc2cc1Nc1cc(C2CC2)n[nH]1 ; COc1cc2c(NS(=O)(=O)c3c(OC)cc(C#N)cc3OC)noc2cc1Nc1cc(C2CC2)n[nH]1 ; COc1cc2c(NS(=O)(=O)c3cc4c(cc3OC)COCC4)noc2cc1Nc1cc(C2CC2)[nH]n1]

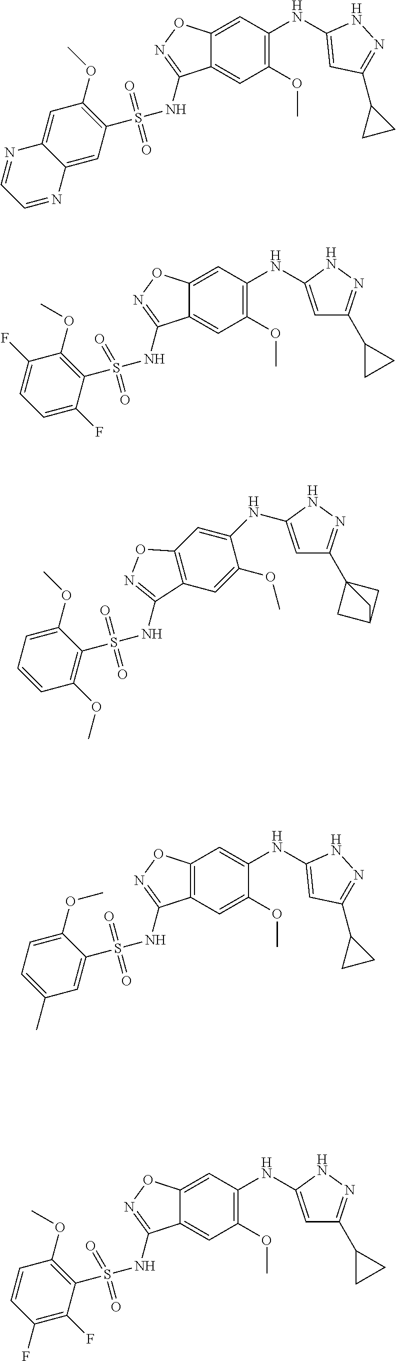 [SMILES: COc1cc2c(NS(=O)(=O)c3cc4nccnc4cc3OC)noc2cc1Nc1cc(C2CC2)n[nH]1 ; COc1cc2c(NS(=O)(=O)c3c(F)ccc(F)c3OC)noc2cc1Nc1cc(C2CC2)n[nH]1 ; COc1cc2c(NS(=O)(=O)c3c(OC)cccc3OC)noc2cc1Nc1cc(C23CC(C2)C3)n[nH]1 ; COc1cc2c(NS(=O)(=O)c3cc(C)ccc3OC)noc2cc1Nc1cc(C2CC2)n[nH]1 ; COc1cc2c(NS(=O)(=O)c3c(OC)ccc(F)c3F)noc2cc1Nc1cc(C2CC2)n[nH]1]

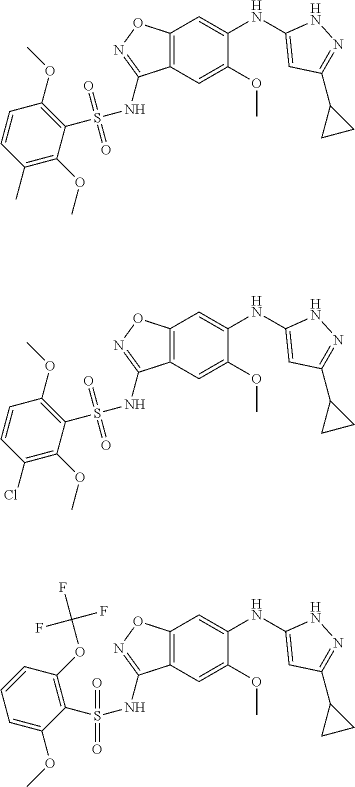 [SMILES: COc1cc2c(NS(=O)(=O)c3c(OC)ccc(C)c3OC)noc2cc1Nc1cc(C2CC2)n[nH]1 ; COc1cc2c(NS(=O)(=O)c3c(OC)ccc(Cl)c3OC)noc2cc1Nc1cc(C2CC2)n[nH]1 ; COc1cc2c(NS(=O)(=O)c3c(OC)cccc3OC(F)(F)F)noc2cc1Nc1cc(C2CC2)n[nH]1]

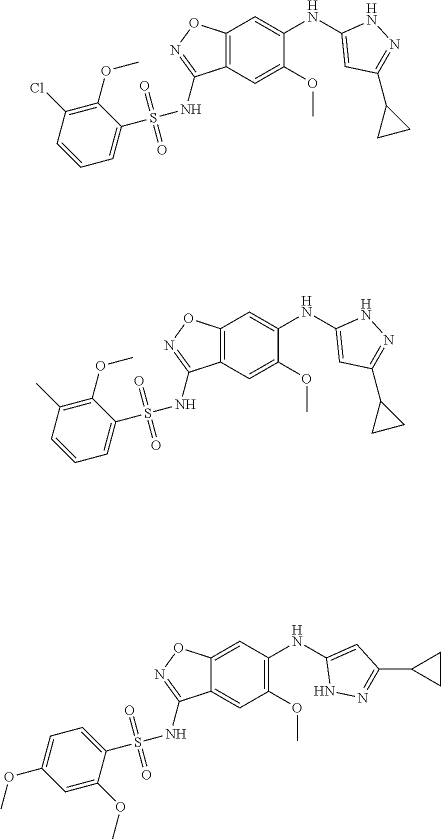 [SMILES: COc1cc2c(NS(=O)(=O)c3cccc(Cl)c3OC)noc2cc1Nc1cc(C2CC2)n[nH]1 ; COc1cc2c(NS(=O)(=O)c3cccc(C)c3OC)noc2cc1Nc1cc(C2CC2)n[nH]1 ; COc1ccc(S(=O)(=O)Nc2noc3cc(Nc4cc(C5CC5)n[nH]4)c(OC)cc23)c(OC)c1]

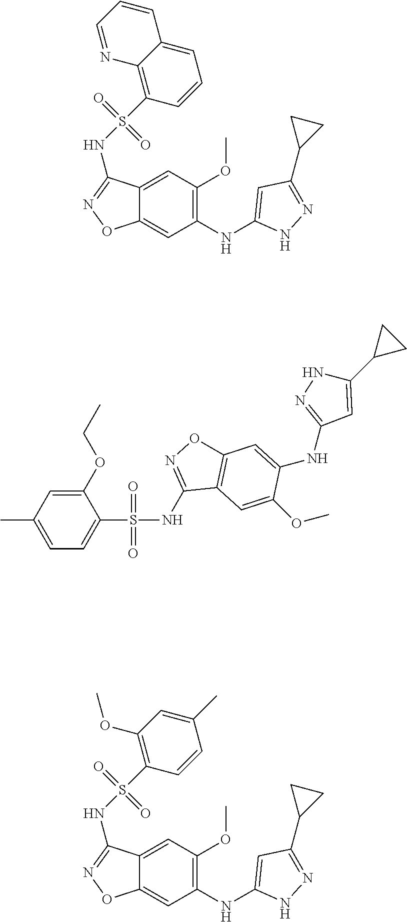 [SMILES: COc1cc2c(NS(=O)(=O)c3cccc4cccnc34)noc2cc1Nc1cc(C2CC2)n[nH]1 ; CCOc1cc(C)ccc1S(=O)(=O)Nc1noc2cc(Nc3cc(C4CC4)[nH]n3)c(OC)cc12 ; COc1cc2c(NS(=O)(=O)c3ccc(C)cc3OC)noc2cc1Nc1cc(C2CC2)n[nH]1]

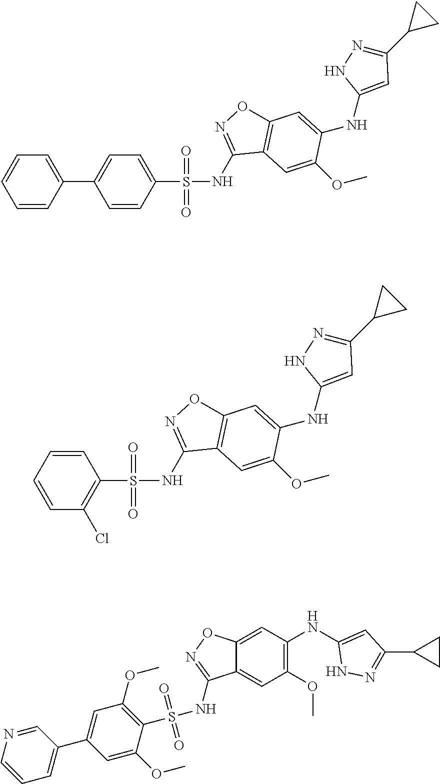 [SMILES: COc1cc2c(NS(=O)(=O)c3ccc(-c4ccccc4)cc3)noc2cc1Nc1cc(C2CC2)n[nH]1 ; COc1cc2c(NS(=O)(=O)c3ccccc3Cl)noc2cc1Nc1cc(C2CC2)n[nH]1 ; COc1cc2c(NS(=O)(=O)c3c(OC)cc(-c4cccnc4)cc3OC)noc2cc1Nc1cc(C2CC2)n[nH]1]

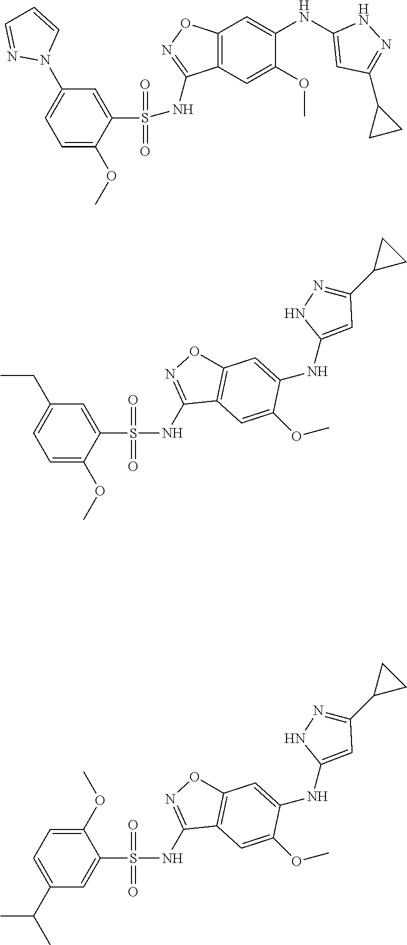 [SMILES: COc1cc2c(NS(=O)(=O)c3cc(-n4cccn4)ccc3OC)noc2cc1Nc1cc(C2CC2)n[nH]1 ; CCc1ccc(OC)c(S(=O)(=O)Nc2noc3cc(Nc4cc(C5CC5)n[nH]4)c(OC)cc23)c1 ; COc1cc2c(NS(=O)(=O)c3cc(C(C)C)ccc3OC)noc2cc1Nc1cc(C2CC2)n[nH]1]

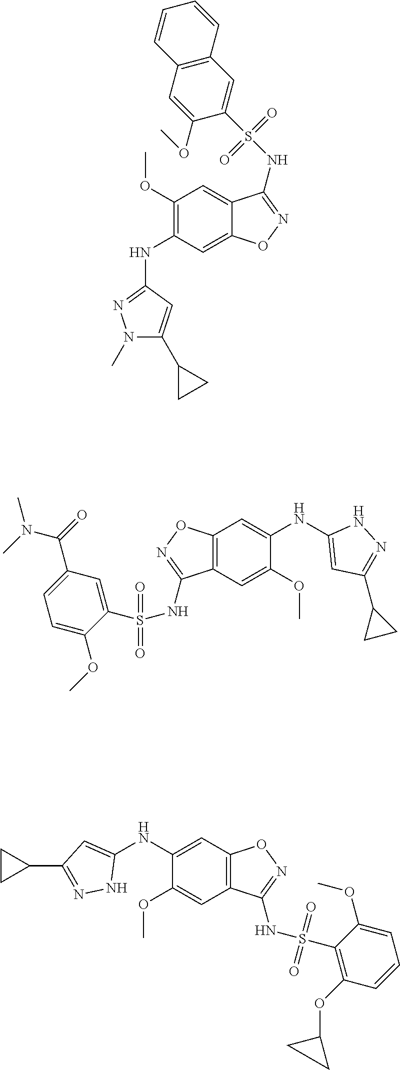 [SMILES: COc1cc2c(NS(=O)(=O)c3cc4ccccc4cc3OC)noc2cc1Nc1cc(C2CC2)n(C)n1 ; COc1cc2c(NS(=O)(=O)c3cc(C(=O)N(C)C)ccc3OC)noc2cc1Nc1cc(C2CC2)n[nH]1 ; COc1cc2c(NS(=O)(=O)c3c(OC)cccc3OC3CC3)noc2cc1Nc1cc(C2CC2)n[nH]1]

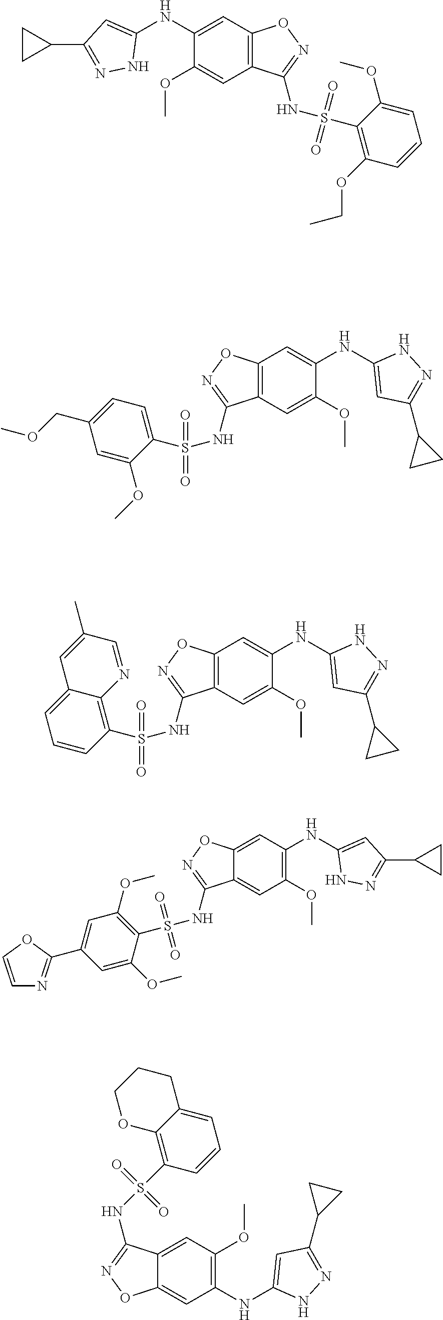 [SMILES: CCOc1cccc(OC)c1S(=O)(=O)Nc1noc2cc(Nc3cc(C4CC4)n[nH]3)c(OC)cc12 ; COCc1ccc(S(=O)(=O)Nc2noc3cc(Nc4cc(C5CC5)n[nH]4)c(OC)cc23)c(OC)c1 ; COc1cc2c(NS(=O)(=O)c3cccc4cc(C)cnc34)noc2cc1Nc1cc(C2CC2)n[nH]1 ; COc1cc2c(NS(=O)(=O)c3c(OC)cc(-c4ncco4)cc3OC)noc2cc1Nc1cc(C2CC2)n[nH]1 ; COc1cc2c(NS(=O)(=O)c3cccc4c3OCCC4)noc2cc1Nc1cc(C2CC2)n[nH]1]

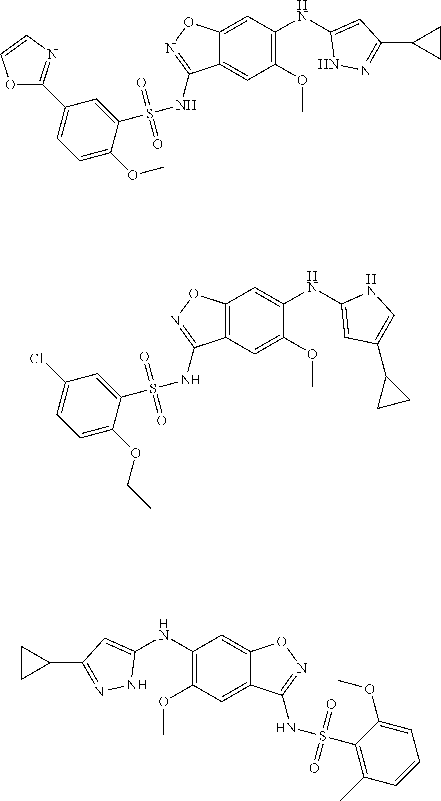 [SMILES: COc1cc2c(NS(=O)(=O)c3cc(-c4ncco4)ccc3OC)noc2cc1Nc1cc(C2CC2)n[nH]1 ; CCOc1ccc(Cl)cc1S(=O)(=O)Nc1noc2cc(Nc3cc(C4CC4)c[nH]3)c(OC)cc12 ; COc1cc2c(NS(=O)(=O)c3c(C)cccc3OC)noc2cc1Nc1cc(C2CC2)n[nH]1]

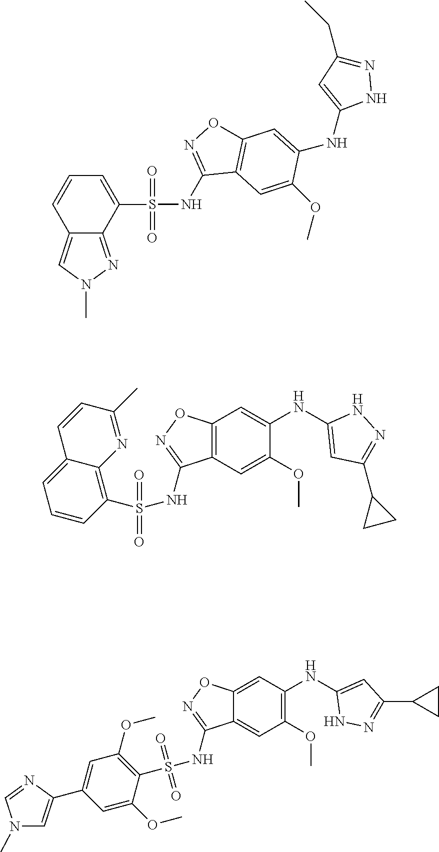 [SMILES: CCc1cc(Nc2cc3onc(NS(=O)(=O)c4cccc5cn(C)nc45)c3cc2OC)[nH]n1 ; COc1cc2c(NS(=O)(=O)c3cccc4ccc(C)nc34)noc2cc1Nc1cc(C2CC2)n[nH]1 ; COc1cc2c(NS(=O)(=O)c3c(OC)cc(-c4cn(C)cn4)cc3OC)noc2cc1Nc1cc(C2CC2)n[nH]1]

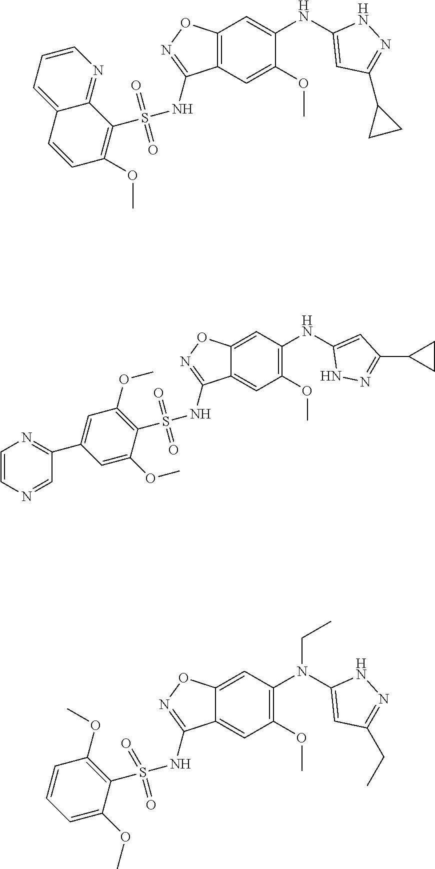 [SMILES: COc1cc2c(NS(=O)(=O)c3c(OC)ccc4cccnc34)noc2cc1Nc1cc(C2CC2)n[nH]1 ; COc1cc2c(NS(=O)(=O)c3c(OC)cc(-c4cnccn4)cc3OC)noc2cc1Nc1cc(C2CC2)n[nH]1 ; CCc1cc(N(CC)c2cc3onc(NS(=O)(=O)c4c(OC)cccc4OC)c3cc2OC)[nH]n1]

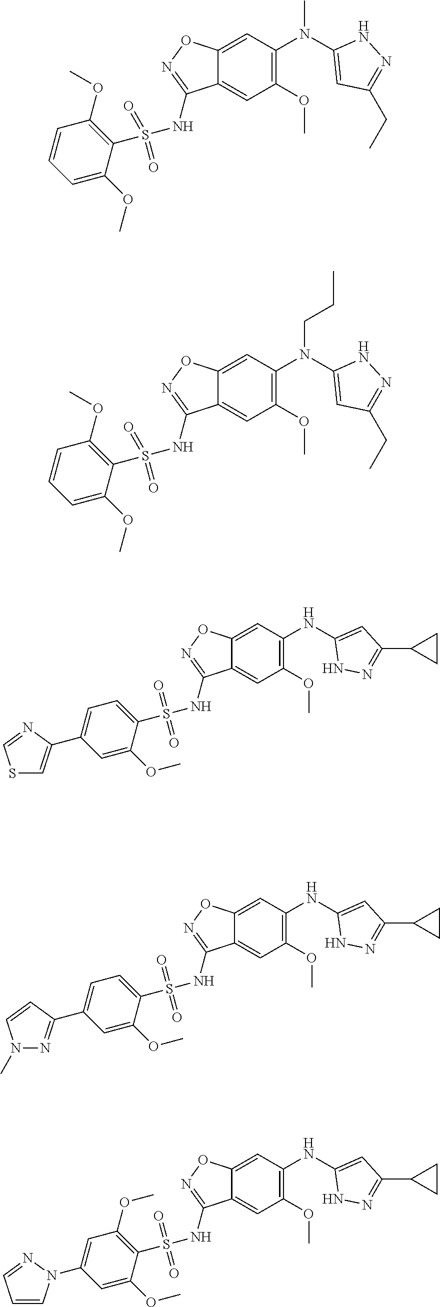 [SMILES: CCc1cc(N(C)c2cc3onc(NS(=O)(=O)c4c(OC)cccc4OC)c3cc2OC)[nH]n1 ; CCCN(c1cc(CC)n[nH]1)c1cc2onc(NS(=O)(=O)c3c(OC)cccc3OC)c2cc1OC ; COc1cc2c(NS(=O)(=O)c3ccc(-c4cscn4)cc3OC)noc2cc1Nc1cc(C2CC2)n[nH]1 ; COc1cc2c(NS(=O)(=O)c3ccc(-c4ccn(C)n4)cc3OC)noc2cc1Nc1cc(C2CC2)n[nH]1 ; COc1cc2c(NS(=O)(=O)c3c(OC)cc(-n4cccn4)cc3OC)noc2cc1Nc1cc(C2CC2)n[nH]1]

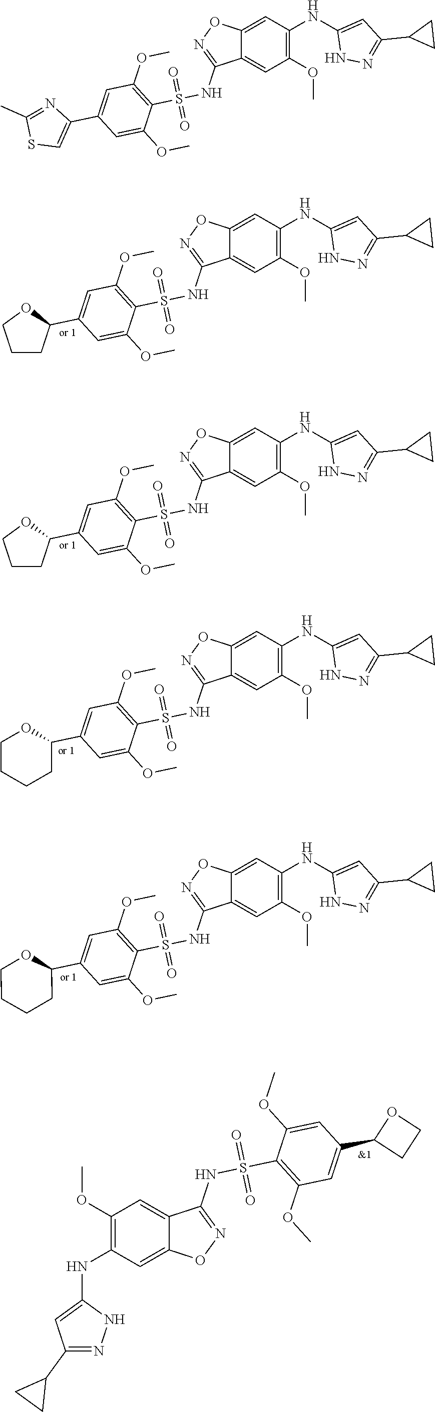 [SMILES: COc1cc2c(NS(=O)(=O)c3c(OC)cc(-c4csc(C)n4)cc3OC)noc2cc1Nc1cc(C2CC2)n[nH]1 ; COc1cc2c(NS(=O)(=O)c3c(OC)cc([C@H]4CCCO4)cc3OC)noc2cc1Nc1cc(C2CC2)n[nH]1 ; COc1cc2c(NS(=O)(=O)c3c(OC)cc([C@@H]4CCCO4)cc3OC)noc2cc1Nc1cc(C2CC2)n[nH]1 ; COc1cc2c(NS(=O)(=O)c3c(OC)cc([C@@H]4CCCCO4)cc3OC)noc2cc1Nc1cc(C2CC2)n[nH]1 ; COc1cc2c(NS(=O)(=O)c3c(OC)cc([C@H]4CCCCO4)cc3OC)noc2cc1Nc1cc(C2CC2)n[nH]1 ; COc1cc2c(NS(=O)(=O)c3c(OC)cc([C@@H]4CCO4)cc3OC)noc2cc1Nc1cc(C2CC2)n[nH]1]

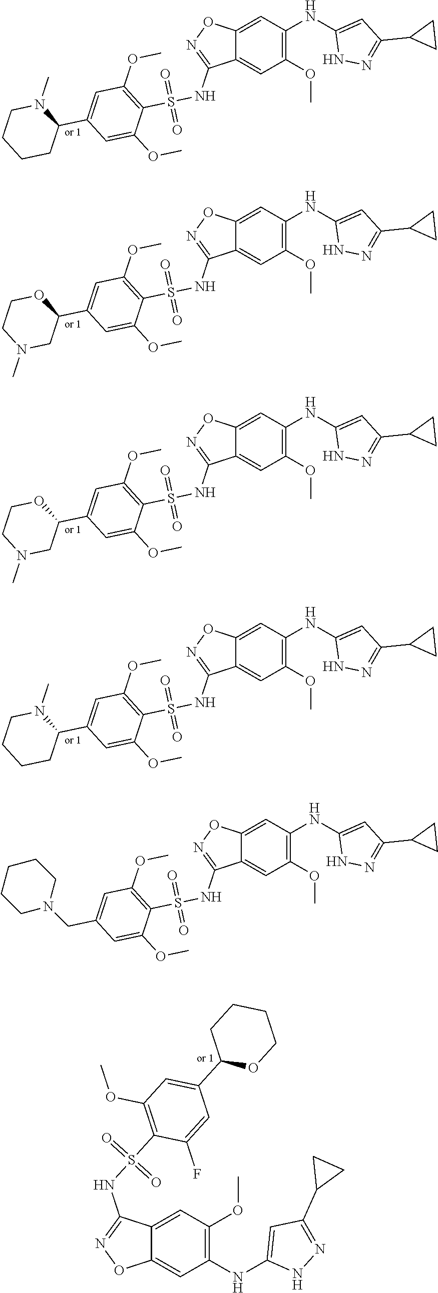 [SMILES: COc1cc2c(NS(=O)(=O)c3c(OC)cc([C@H]4CCCCN4C)cc3OC)noc2cc1Nc1cc(C2CC2)n[nH]1 ; COc1cc2c(NS(=O)(=O)c3c(OC)cc([C@H]4CN(C)CCO4)cc3OC)noc2cc1Nc1cc(C2CC2)n[nH]1 ; COc1cc2c(NS(=O)(=O)c3c(OC)cc([C@@H]4CN(C)CCO4)cc3OC)noc2cc1Nc1cc(C2CC2)n[nH]1 ; COc1cc2c(NS(=O)(=O)c3c(OC)cc([C@@H]4CCCCN4C)cc3OC)noc2cc1Nc1cc(C2CC2)n[nH]1 ; COc1cc2c(NS(=O)(=O)c3c(OC)cc(CN4CCCCC4)cc3OC)noc2cc1Nc1cc(C2CC2)n[nH]1 ; COc1cc2c(NS(=O)(=O)c3c(F)cc([C@H]4CCCCO4)cc3OC)noc2cc1Nc1cc(C2CC2)n[nH]1]

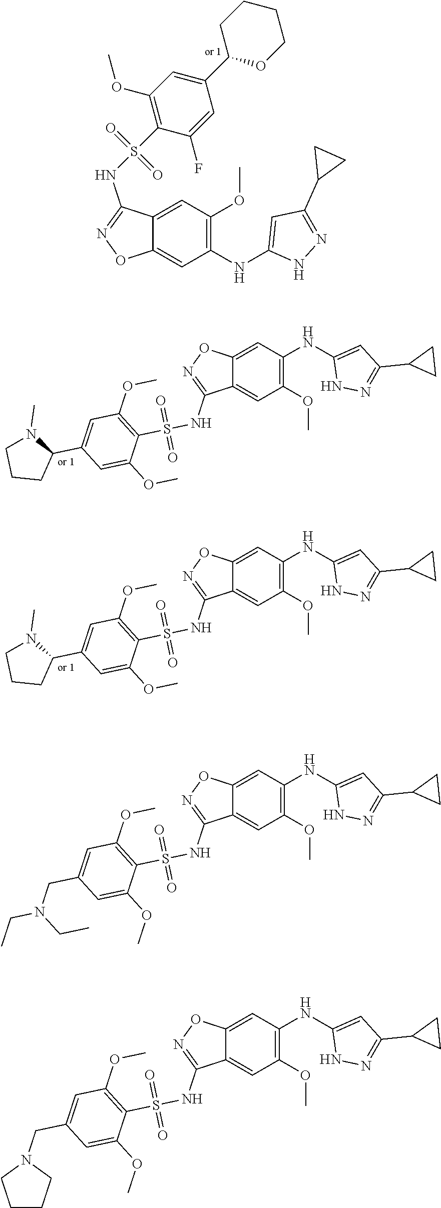 [SMILES: COc1cc2c(NS(=O)(=O)c3c(F)cc([C@@H]4CCCCO4)cc3OC)noc2cc1Nc1cc(C2CC2)n[nH]1 ; COc1cc2c(NS(=O)(=O)c3c(OC)cc([C@H]4CCCN4C)cc3OC)noc2cc1Nc1cc(C2CC2)n[nH]1 ; COc1cc2c(NS(=O)(=O)c3c(OC)cc([C@@H]4CCCN4C)cc3OC)noc2cc1Nc1cc(C2CC2)n[nH]1 ; CCN(CC)Cc1cc(OC)c(S(=O)(=O)Nc2noc3cc(Nc4cc(C5CC5)n[nH]4)c(OC)cc23)c(OC)c1 ; COc1cc2c(NS(=O)(=O)c3c(OC)cc(CN4CCCC4)cc3OC)noc2cc1Nc1cc(C2CC2)n[nH]1]

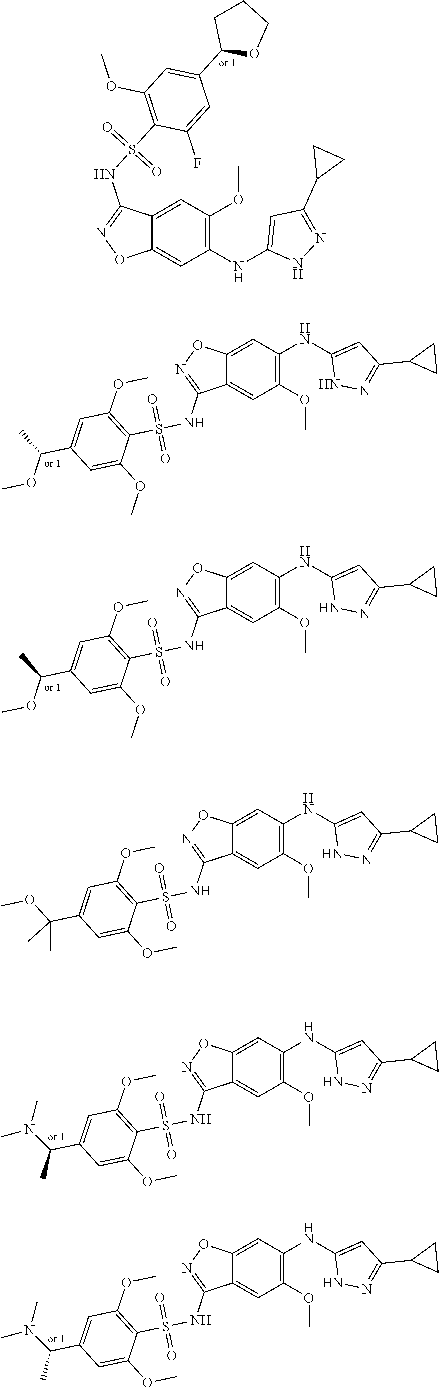 [SMILES: COc1cc2c(NS(=O)(=O)c3c(F)cc([C@H]4CCCO4)cc3OC)noc2cc1Nc1cc(C2CC2)n[nH]1 ; COc1cc2c(NS(=O)(=O)c3c(OC)cc([C@@H](C)OC)cc3OC)noc2cc1Nc1cc(C2CC2)n[nH]1 ; COc1cc2c(NS(=O)(=O)c3c(OC)cc([C@H](C)OC)cc3OC)noc2cc1Nc1cc(C2CC2)n[nH]1 ; COc1cc2c(NS(=O)(=O)c3c(OC)cc(C(C)(C)OC)cc3OC)noc2cc1Nc1cc(C2CC2)n[nH]1 ; COc1cc2c(NS(=O)(=O)c3c(OC)cc([C@@H](C)N(C)C)cc3OC)noc2cc1Nc1cc(C2CC2)n[nH]1 ; COc1cc2c(NS(=O)(=O)c3c(OC)cc([C@H](C)N(C)C)cc3OC)noc2cc1Nc1cc(C2CC2)n[nH]1]

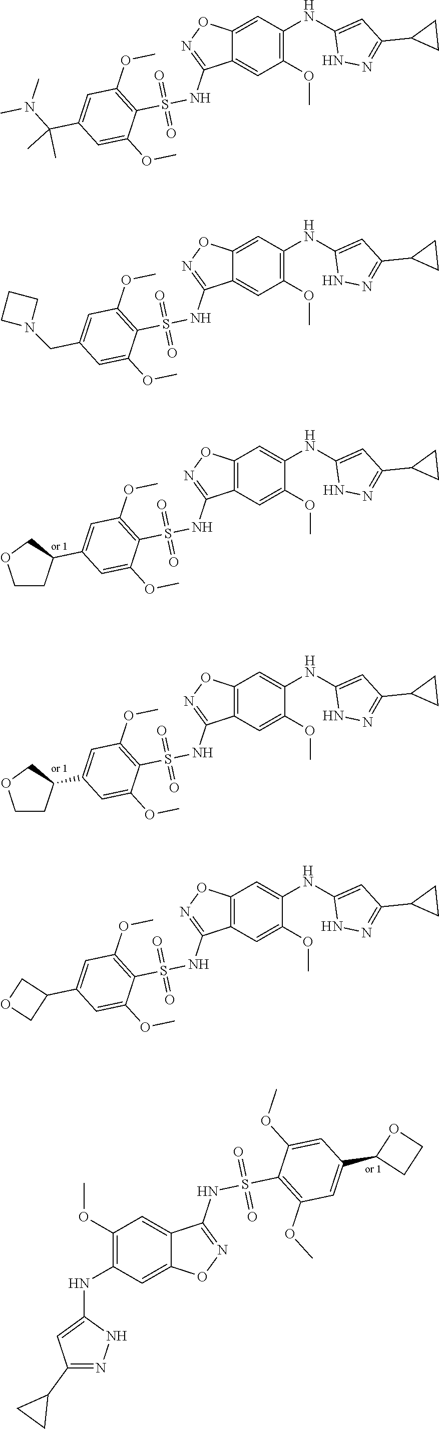

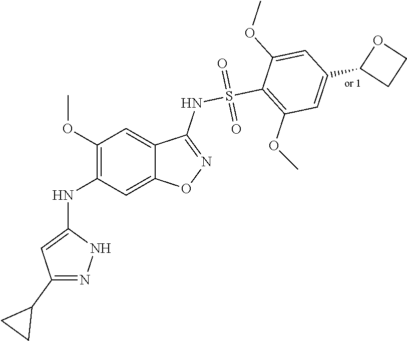 [SMILES: COc1cc2c(NS(=O)(=O)c3c(OC)cc([C@H]4CCO4)cc3OC)noc2cc1Nc1cc(C2CC2)n[nH]1]

A “pharmaceutical composition” refers to a mixture of one or more of the compounds of the invention, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the invention. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, as used herein, unless otherwise indicated, means reversing, alleviating, or inhibiting the progress of the disease, disorder or condition to which such term applies, or one or more symptoms of such disease, disorder or condition. The term “treatment”, as used herein, unless otherwise indicated, refers to the act of treating as “treating” is defined immediately above.

As used herein, the term, “subject, “individual” or “patient,” used interchangeably, refers to any animal, including mammals. Mammals according to the invention include canine, feline, bovine, caprine, equine, ovine, porcine, rodents, lagomorphs, primates, humans and the like, and encompass mammals in utero. In an embodiment, humans are preferred subjects. Human subjects may be of any gender and at any stage of development.

As used herein, the phrase “therapeutically effective amount” or “effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

### Salts

Salts encompassed within the term “pharmaceutically acceptable salts” refer to the compounds of this invention which are generally prepared by reacting the free base or free acid with a suitable organic or inorganic acid, or a suitable organic or inorganic base, respectively, to provide a salt of the compound of the invention that is suitable for administration to a subject or patient.

In addition, the compounds of Formulas (I)-(X), (Ia)-(VIIa), and (Ib)-(VIIb) may also include other salts of such compounds which are not necessarily pharmaceutically acceptable salts, which may be useful as intermediates for one or more of the following: 1) preparing compounds of Formulas (I)-(X), (Ia)-(VIIa), and (Ib)-(VIIb); 2) purifying compounds of Formulas (I)-(X), (Ia)-(VIIa), and (Ib)-(VIIb); 3) separating enantiomers of compounds of Formulas (I)-(X), (Ia)-(VIIa), and (Ib)-(VIIb); or 4) separating diastereomers of compounds of Formulas (I)-(X), (Ia)-(VIIa), and (Ib)-(VIIb).

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lithium, lysine, magnesium, meglumine, olamine, piperazine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50 (26), 6665-6672.

Pharmaceutically acceptable salts of compounds of the invention may be prepared by methods well known to one skilled in the art, including but not limited to the following procedures:
(i) by reacting a compound of the invention with the desired acid or base;
(ii) by removing an acid-or base-labile protecting group from a suitable precursor of a compound of the invention or by ring-opening a suitable cyclic precursor, for example, a lactone or lactam, using the desired acid or base; or
(iii) by converting one salt of a compound of the invention to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the invention, and pharmaceutically acceptable salts thereof, may exist in unsolvated and solvated forms. The term ‘solvate’ is used herein to describe a molecular complex comprising the compound of the invention, or a pharmaceutically acceptable salt thereof, and one or more pharmaceutically acceptable solvent molecules, for example, ethanol. The term ‘hydrate’ is employed when said solvent is water.

In addition, the compounds of Formulas (I)-(X), (Ia)-(VIIa), and (Ib)-(VIIb) may also include other solvates of such compounds which are not necessarily pharmaceutically acceptable solvates, which may be useful as intermediates for one or more of the following: 1) preparing compounds of Formulas (I)-(X), (Ia)-(VIIa), and (Ib)-(VIIb); 2) purifying compounds of Formulas (I)-(X), (Ia)-(VIIa), and (Ib)-(VIIb); 3) separating enantiomers of compounds of Formulas (I)-(X), (Ia)-(VIIa), and (Ib)-(VIIb); or 4) separating diastereomers of compounds of Formulas (I)-(X), (Ia)-(VIIa), and (Ib)-(VIIb).

A currently accepted classification system for organic hydrates is one that defines isolated site, channel, or metal-ion coordinated hydrates-see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together-see Chem Commun, 17; 1889-1896, by O. Almarsson and M. J. Zaworotko (2004). For a general review of multi-component complexes, see J Pharm Sci, 64 (8), 1269-1288, by Haleblian (August 1975).

### Solid Form

The compounds of the invention may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the invention may also exist in a mesomorphic state (mesophase or liquid crystal) when subjected to suitable conditions. The mesomorphic state is intermediate between the true crystalline state and the true liquid state (either melt or solution) and consists of two-dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4th Edition (Edward Arnold, 1970).

### Stereoisomers

Compounds of the invention may exist as two or more stereoisomers. Stereoisomers of the compounds may include cis and trans isomers (geometric isomers), optical isomers such as Rand S enantiomers, diastereomers, rotational isomers, atropisomers, and conformational isomers. For example, compounds of the invention containing one or more asymmetric carbon atoms may exist as two or more stereoisomers. Cis/trans isomers may also exist for saturated rings.

The pharmaceutically acceptable salts of compounds of the invention may also contain a counterion which is optically active (e.g., d-lactate or l-lysine) or racemic (e.g., dl-tartrate or di-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the invention contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the invention (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art-see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the invention containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the invention have been drawn herein in a single tautomeric form, all possible tautomeric forms are included within the scope of the invention.

Tautomerism in the compounds of the invention may be depicted as shown in the structure below.

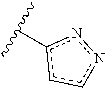

The dotted line in the structure above means that the tautomeric forms are in resonance, which depicts movement of only electrons. Resonance is the presence of more than one form (of the same chemical compound) which determines the actual structure of a compound.

The structure:

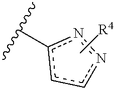

is equivalent to

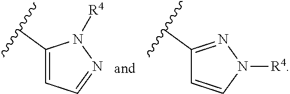

Compounds of the invention having the substituents

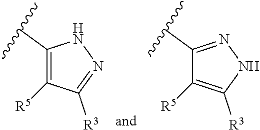

are tautomers.

### Isotopes

The present invention includes all pharmaceutically acceptable isotopically-labeled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the invention, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the invention may be defined by the deuterium enrichment factor. It is understood that one or more deuterium may exchange with hydrogen under physiological conditions.

In some embodiments, one or more hydrogen atoms on certain metabolic sites on the compounds of the invention are deuterated. Certain metabolic sites on the compounds of the invention are depicted below.

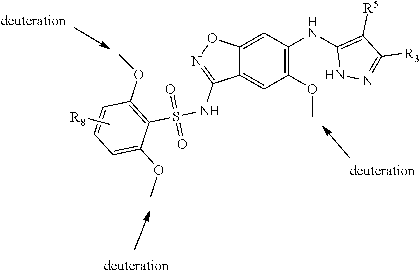

In some embodiments, the deuterium compound is selected from any one of the compounds set forth in Tables 1-12 shown in the Examples section.

TABLE 1

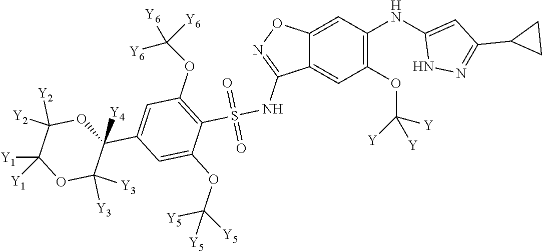

| Example | | | | | | | |
|---|---|---|---|---|---|---|---|
| Number | Y₁ | Y₂ | Y₃ | Y₄ | Y₅ | Y₆ | Y₇ |
| 145-D1 | D | H | H | H | H | H | H |
| 145-D2 | H | D | H | H | H | H | H |
| 145-D3 | H | H | H | H | D | H | H |
| 145-D4 | H | H | H | H | H | D | H |
| 145-D5 | H | H | H | H | H | H | D |
| 145-D6 | H | H | D | H | H | H | H |
| 145-D7 | H | H | H | D | H | H | H |

TABLE 2

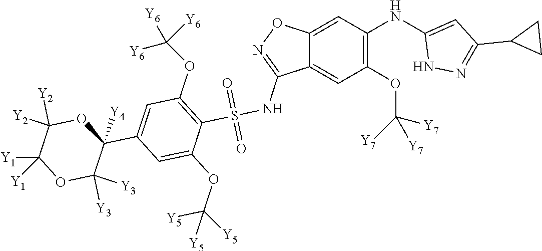

| Example | | | | | | | |
|---|---|---|---|---|---|---|---|
| Number | Y₁ | Y₂ | Y₃ | Y₄ | Y₅ | Y₆ | Y₇ |
| 146-D1 | D | H | H | H | H | H | H |
| 146-D2 | H | D | H | H | H | H | H |
| 146-D3 | H | H | H | H | D | H | H |
| 146-D4 | H | H | H | H | H | D | H |
| 146-D5 | H | H | H | H | H | H | D |
| 146-D6 | H | H | D | H | H | H | H |
| 146-D7 | H | H | H | D | H | H | H |

TABLE 3

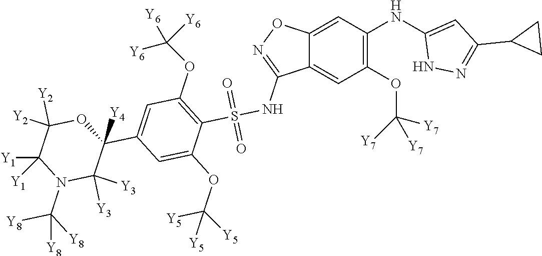

| Example | | | | | | | | |
|---|---|---|---|---|---|---|---|---|
| Number | Y₁ | Y₂ | Y₃ | Y₄ | Y₅ | Y₆ | Y₇ | Y₈ |
| 152-D1 | H | H | H | H | H | H | H | D |
| 152-D2 | D | H | H | H | H | H | H | H |
| 152-D3 | H | D | H | H | H | H | H | H |
| 152-D4 | H | H | H | D | H | H | H | H |
| 152-D5 | H | H | H | H | D | H | H | H |
| 152-D6 | H | H | H | H | H | D | H | H |
| 152-D7 | H | H | H | H | H | H | D | H |

TABLE 4

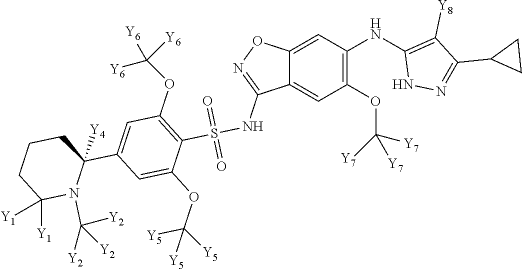

| Example | | | | | | | |
|---|---|---|---|---|---|---|---|
| Number | Y₁ | Y₂ | Y₄ | Y₅ | Y₆ | Y₇ | Y₈ |
| 174-D1 | H | D | H | H | H | H | H |
| 174-D2 | H | H | H | D | H | H | H |
| 174-D3 | H | H | H | H | D | H | H |
| 174-D4 | D | H | H | H | H | H | H |
| 174-D5 | H | H | H | H | H | D | H |
| 174-D6 | H | H | D | H | H | H | H |
| 174-D7 | H | H | H | H | H | H | D |

TABLE 5

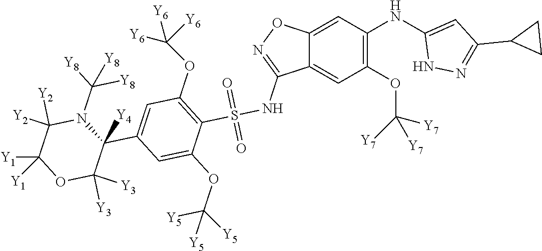

| Example | | | | | | | | |
|---|---|---|---|---|---|---|---|---|
| Number | Y₁ | Y₂ | Y₃ | Y₄ | Y₅ | Y₆ | Y₇ | Y₈ |
| 179-D1 | H | H | H | H | H | H | H | D |
| 179-D2 | D | H | H | H | H | H | H | H |
| 179-D3 | H | H | H | H | D | H | H | H |
| 179-D4 | H | H | H | H | H | D | H | H |
| 179-D5 | H | D | H | H | H | H | H | H |
| 179-D6 | H | H | H | H | H | H | D | H |
| 179-D7 | H | H | D | H | H | H | H | H |
| 179-D8 | H | H | H | D | H | H | H | H |

TABLE 6

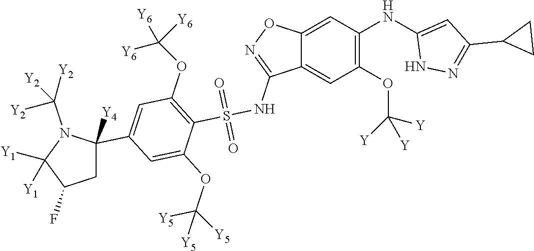

| Example | | | | | | | | |
|---|---|---|---|---|---|---|---|---|
| Number | Y₁ | Y₂ | Y₃ | Y₄ | Y₅ | Y₆ | Y₇ | Y₈ |
| 181-D1 | H | D | H | H | H | H | H | H |
| 181-D2 | H | H | H | H | D | H | H | H |
| 181-D3 | H | H | H | H | H | D | H | H |
| 181-D4 | D | H | H | H | H | H | H | H |
| 181-D5 | H | H | H | H | H | H | D | H |
| 181-D6 | H | H | H | D | H | H | H | H |

TABLE 7

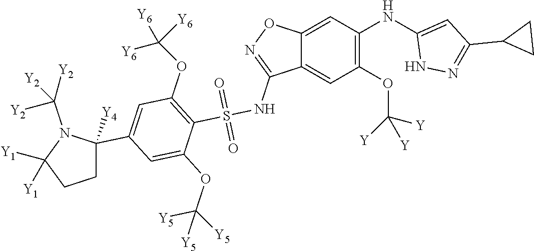

| Example | | | | | | | | |
|---|---|---|---|---|---|---|---|---|
| Number | Y₁ | Y₂ | Y₃ | Y₄ | Y₅ | Y₆ | Y₇ | Y₈ |
| 188-D1 | H | D | H | H | H | H | H | H |
| 188-D2 | D | H | H | H | H | H | H | H |
| 188-D3 | H | H | H | H | D | H | H | H |
| 188-D4 | H | H | H | H | H | D | H | H |
| 188-D5 | H | H | H | H | H | H | D | H |
| 188-D6 | H | H | H | D | H | H | H | H |

TABLE 8

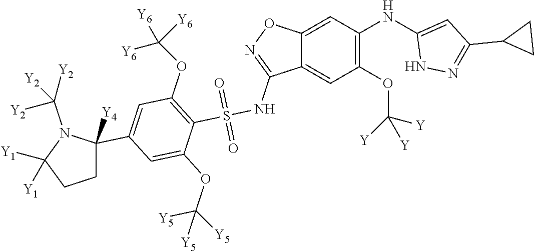

| Example | | | | | | | | |
|---|---|---|---|---|---|---|---|---|
| Number | Y₁ | Y₂ | Y₃ | Y₄ | Y₅ | Y₆ | Y₇ | Y₈ |
| 189-D1 | H | D | H | H | H | H | H | H |
| 189-D2 | D | H | H | H | H | H | H | H |
| 189-D3 | H | H | H | H | D | H | H | H |
| 189-D4 | H | H | H | H | H | D | H | H |
| 189-D5 | H | H | H | H | H | H | D | H |
| 189-D6 | H | H | H | D | H | H | H | H |

TABLE 9

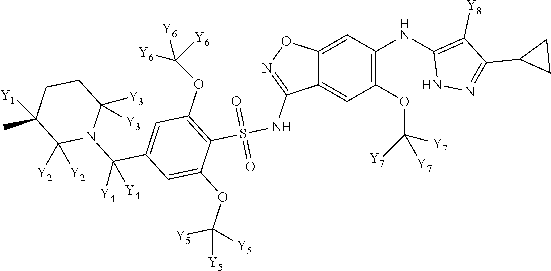

| Example | | | | | | | | |
|---|---|---|---|---|---|---|---|---|
| Number | Y₁ | Y₂ | Y₃ | Y₄ | Y₅ | Y₆ | Y₇ | Y₈ |
| 195-D1 | D | H | H | H | H | H | H | H |
| 195-D2 | H | H | H | D | H | H | H | H |
| 195-D3 | H | H | H | H | D | H | H | H |
| 195-D4 | H | H | H | H | H | D | H | H |
| 195-D5 | H | H | D | H | H | H | H | H |
| 195-D6 | H | D | H | H | H | H | H | H |

TABLE 10

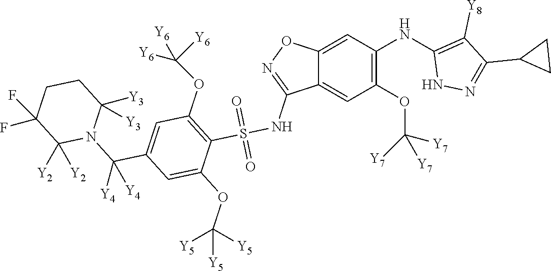

| Example | | | | | | | | |
|---|---|---|---|---|---|---|---|---|
| Number | Y₁ | Y₂ | Y₃ | Y₄ | Y₅ | Y₆ | Y₇ | Y₈ |
| 199-D1 | H | H | H | D | H | H | H | H |
| 199-D2 | H | H | D | H | H | H | H | H |
| 199-D3 | H | H | H | H | D | H | H | H |
| 199-D4 | H | H | H | H | H | D | H | H |
| 199-D5 | H | D | H | H | H | H | H | H |
| 199-D6 | H | H | H | H | H | H | D | H |
| 199-D7 | H | H | H | H | H | H | H | D |

TABLE 11

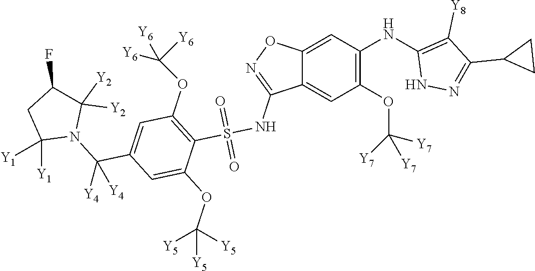

| Example | | | | | | | | |
|---|---|---|---|---|---|---|---|---|
| Number | Y₁ | Y₂ | Y₃ | Y₄ | Y₅ | Y₆ | Y₇ | Y₈ |
| 201-D1 | H | H | H | D | H | H | H | H |
| 201-D2 | H | H | H | H | D | H | H | H |
| 201-D3 | H | H | H | H | H | D | H | H |
| 201-D4 | D | H | H | H | H | H | H | H |
| 201-D5 | H | D | H | H | H | H | H | H |
| 201-D6 | H | H | H | H | H | H | D | H |
| 201-D7 | H | H | H | H | H | H | H | D |

TABLE 12

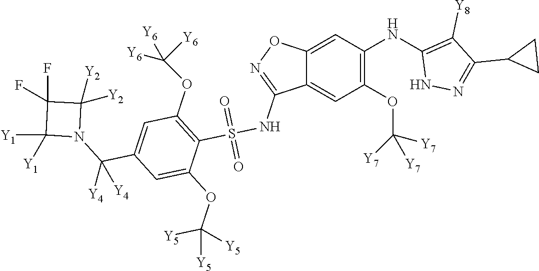

| Example | | | | | | | | |
|---|---|---|---|---|---|---|---|---|
| Number | Y₁ | Y₂ | Y₃ | Y₄ | Y₅ | Y₆ | Y₇ | Y₈ |
| 203-D1 | H | H | H | D | H | H | H | H |
| 203-D2 | D | H | H | H | H | H | H | H |
| 203-D3 | H | D | H | H | H | H | H | H |
| 203-D4 | H | H | H | H | D | H | H | H |
| 203-D5 | H | H | H | H | H | D | H | H |
| 203-D6 | H | H | H | H | H | H | D | H |
| 203-D7 | H | H | H | H | H | H | H | D |

TABLE 13

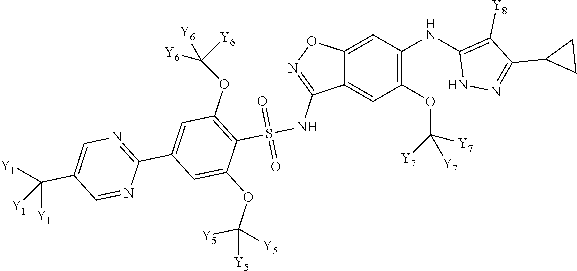

| Example | | | | | |
|---|---|---|---|---|---|
| Number | Y₁ | Y₅ | Y₆ | Y₇ | Y₈ |
| 213-D1 | D | H | H | H | H |
| 213-D2 | D | H | H | H | H |
| 213-D3 | H | H | H | H | H |
| 213-D4 | H | D | H | H | H |
| 213-D5 | H | H | D | H | H |
| 213-D6 | H | H | H | D | H |
| 213-D7 | H | H | H | H | D |

Isotopically-labeled compounds of the invention may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g., D₂O, d₆-acetone, d₆-DMSO.

### Prodrugs

A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; or (c) an amide, imine, carbamate or amine derivative of an amino group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂—(e.g., ¹BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀) alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

### Pharmaceutical Compositions

In another embodiment, the invention comprises pharmaceutical compositions. For pharmaceutical composition purposes, the compound per se or pharmaceutically acceptable salt thereof will simply be referred to as the compounds of the invention.

The compositions of this invention may be in a variety of forms. These include, for example, semi-solid and solid dosage forms, such as dispersions or suspensions, tablets, capsules, and pills. The form depends on the intended mode of administration and therapeutic application.

Oral administration of a solid dosage form may be, for example, presented in discrete units, such as hard or soft capsules, pills, cachets, lozenges, or tablets, each containing a predetermined amount of at least one compound of the invention. In such solid dosage forms, the compounds of the invention are ordinarily combined with one or more adjuvants. Such capsules or tablets may comprise a controlled release formulation. In the case of capsules, tablets, and pills, the dosage forms also may comprise buffering agents or may be prepared with enteric coatings.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the invention may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the invention may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the invention, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

A drug product intermediate (DPI) is a partly processed material that must undergo further processing steps before it becomes bulk drug product. Compounds of the invention may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the invention isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the invention and a polymer excipient. Other excipients as well as concentrations of said excipients and the compound of the invention are well known in the art and are described in standard textbooks. See, for example, “Amorphous Solid Dispersions Theory and Practice” by Navnit Shah et al.

### Administration and Dosing

Typically, a compound of the invention is administered in an amount effective to treat a condition as described herein. The compounds of the invention may be administered as compound per se, or alternatively, as a pharmaceutically acceptable salt. For administration and dosing purposes, the compound per se or pharmaceutically acceptable salt thereof will simply be referred to as the compounds of the invention.

The compounds of the invention are administered by any suitable route in the form of a pharmaceutical composition adapted to such a route, and in a dose effective for the treatment intended.

The compounds of the invention may be administered orally. Oral administration may involve swallowing, so that the compound enters the gastrointestinal tract, or buccal or sublingual administration may be employed by which the compound enters the bloodstream directly from the mouth.

The dosage regimen for the compounds of the invention or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the invention is typically from about 0.01 to about 100 mg/kg (i.e., mg compound of the invention per kg body weight) for the treatment of the indicated conditions discussed herein. In another embodiment, total daily dose of the compound of the invention is from about 0.1 to about 50 mg/kg, and in another embodiment, from about 0.5 to about 30 mg/kg. It is not uncommon that the administration of the compounds of the invention will be repeated a plurality of times in a day (typically no greater than 4 times). Multiple doses per day typically may be used to increase the total daily dose, if desired.

### Therapeutic Methods and Uses

The compounds of the invention act as Lysine Acetyl Transferase (KAT) inhibitors of the MYST family and may be useful in the treatment of abnormal cell growth, such as cancer. In particular, such compounds act as inhibitor of KAT6 and/or KAT7.

The references to the methods of treatment by therapy of this description are to be interpreted as being also references to the compound(s), pharmaceutical compositions and medicaments of the present invention for use in those methods, including use in the manufacture of a medicament for use in those method.

“Abnormal cell growth” or “cancer” as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; (5) benign and malignant cells of other proliferative diseases in which aberrant serine/threonine kinase activation occurs; (6) any tumors that proliferate by aberrant signaling, metabolic, epigenetic and transcriptional mechanism; and (7) benign and malignant cells of other proliferative diseases in which aberrant signaling, metabolic, epigenetic and transcriptional mechanism occur.

For convenience, certain well-known abbreviations, may be used herein, including: estrogen receptor positive (ER+), human epidermal growth factor receptor 2 negative (HER2−), non-small cell lung cancer (NSCLC) and castration resistant prostate cancer (CRPC).

Additional embodiments relate to methods of treating cancer in a subject in need thereof comprising administering to the subject an amount of a compound described herein that is effective in treating cancer.

In another embodiment, the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, and pituitary adenoma, or a combination of two or more of the foregoing cancers.

In another embodiment, the cancer is breast, lung, colon, brain, prostate, stomach, pancreatic, ovarian, melanoma, endocrine, uterine, testicular, or bladder.

In another embodiment, the cancer is breast, lung, prostate, pancreatic, or ovarian.

In another embodiment, the cancer is breast cancer.

In another embodiment, the breast cancer is ER+ breast cancer.

In another embodiment, the breast cancer is ER+ HER2− breast cancer.

In another embodiment, the breast cancer is locally advanced or metastatic ER+ HER2− breast cancer.

In another embodiment, the lung cancer is non-small cell lung cancer.

In another embodiment, the lung cancer is locally advanced or metastatic non-small cell lung cancer.

In another embodiment, the prostate cancer is castration resistant prostate cancer.

In another embodiment, the prostate cancer is locally advanced or metastatic castration resistant prostate cancer.

Additional embodiments relate to methods of treating hematologic tumors in a subject. Some embodiments relate to the treatment of hematologic tumors in a subject in need thereof comprising administering to the subject an amount of a compound described herein that is effective in treating the hematologic tumor.

In another embodiment, the hematologic tumor is leukemia, lymphoma or multiple myeloma.

In another embodiment, the hematologic tumor is leukemia or lymphoma.

Further embodiments relate to methods of treating cancer in a patient which comprises administering to the patient an amount of a compound described herein that is effective in treating cancer in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

More embodiments relate to pharmaceutical compositions for treating cancer in a patient comprising an amount of a compound described herein that is effective in treating cancer, and a pharmaceutically acceptable carrier.

Yet more embodiments relate to a method of treating a disorder associated with angiogenesis in a patient, including a human, comprising administering to said patient an amount of a compound described herein, as defined above, or a pharmaceutically acceptable salt, solvate, hydrate or prodrug thereof, that is effective in treating said disorder in combination with one or more anti-tumor agents listed above. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

Some embodiments relate to a method of (and to a pharmaceutical composition for) treating cancer in a patient which comprise an amount of a compound described herein, or a pharmaceutically acceptable salt, solvate, or hydrate thereof, in combination with an amount of one or more substances selected from anti-angiogenesis agents, signal transduction inhibitors (e.g., inhibiting the means by which regulatory molecules that govern the fundamental processes of cell growth, differentiation, and survival communicated within the cell), and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound described herein in the methods and pharmaceutical compositions described herein.

Tyrosine kinase inhibitors can also be combined with a compound described herein.

VEGF inhibitors, for example, sutent and axitinib, can also be combined with a compound described herein.

ErbB2 receptor inhibitors may be administered in combination with a compound described herein. Various other compounds, such as styrene derivatives, have also been shown to possess tyrosine kinase inhibitory properties, and some of tyrosine kinase inhibitors have been identified as erbB2 receptor inhibitors.

Epidermal growth factor receptor (EGFR) inhibitors may be administered in combination with a compound of the present invention.

PI3K inhibitors, such as PI3K alpha or PI3K beta inhibitors, may be administered in combination with a compound of the present invention.

Mammalian target of rapamycin (mTOR) inhibitors may be administered in combination with a compound of the present invention.

c-Met inhibitors may be administered in combination with a compound of the present invention.

CDK inhibitors may be administered in combination with a compound of the present invention.

MEK inhibitors may be administered in combination with a compound of the present invention.

PARP inhibitors may be administered in combination with a compound of the present invention.

JAK inhibitors may be administered in combination with a compound of the present invention.

An antagonist of a Programmed Death 1 protein (PD-1) may be administered in combination with a compound of the present invention.

An antagonist of Programmed Death-Ligand 1 (PD-L1) may be administered in combination with a compound of the present invention.

Other antiproliferative agents that may be used with the compounds described herein include inhibitors of the enzyme farnesyl protein transferase and inhibitors of the receptor tyrosine kinase PDGFr.

A compound described herein may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase.

A compound described herein may be applied as a sole therapy or may involve one or more other anti-tumor substances, for example those selected from, for example, mitotic inhibitors, alkylating agents, anti-metabolites, growth factor inhibitors, cell cycle inhibitors, intercalating antibiotics, enzymes, and anti-hormones.

The compounds described herein may be used alone or in combination with one or more of a variety of anti-cancer agents or supportive care agents. For example, the compounds described herein may be used with cytotoxic agents. Some embodiments also contemplate the use of the compounds described herein together with hormonal therapy. Further, some embodiments provide a compound described herein alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds described herein may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds described herein.

### Co-Administration

The compounds of the invention may be used alone, or in combination with one or more other therapeutic agents. The invention provides any of the uses, methods or compositions as defined herein wherein a compound of the invention, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the invention and the one or more other therapeutic agents may be administered as a fixed or non-fixed combination of the active ingredients. The term “fixed combination” means a compound of the invention, or a pharmaceutically acceptable salt thereof, and the one or more therapeutic agents, are both administered to a subject simultaneously in a single composition or dosage. The term “non-fixed combination” means that a compound of the invention, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

These agents and compounds of the invention may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising the compound of the invention or pharmaceutical compositions comprising the compound of the invention. A kit may include, in addition to the compound of the invention or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent.

In yet another embodiment, the invention comprises kits that are suitable for use in performing the methods of treatment described herein. In one embodiment, the kit contains a first dosage form comprising one or more of the compounds of the invention in quantities sufficient to carry out the methods of the invention. In another embodiment, the kit comprises one or more compounds of the invention in quantities sufficient to carry out the methods of the invention and a container for the dosage.

### Synthetic Methods

Compounds of the present invention may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds of general scientific interest or previously identified to satisfy a commercial need. Accordingly, such compounds may be one or more of: 1) commercially available; 2) reported in the literature; or 3) prepared from other commonly available substances by one skilled in the art using materials which have been reported in the literature.

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the invention. It will be further appreciated that it may be necessary or desirable to carry out the transformations in a different order from that described in the schemes, or to modify one or more of the transformations, to provide the desired compound of the invention.

In the preparation of compounds of the invention it is noted that some of the preparation methods useful for the preparation of the compounds described herein may require protection of remote functionality (e.g., a primary amine, secondary amine, carboxyl, etc. in a precursor of a compound of the invention). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains an amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the invention.

### General Experimental Details

In the non-limiting Examples and Preparations that illustrate the invention and that are set out in the Description, and in the following General Methods and Schemes, the following abbreviations, definitions and analytical procedures may be referred to:

Unless otherwise noted, materials were obtained from commercial suppliers and were used without further purification. Removal of solvent under reduced pressure or concentration refers to distillation using Büchi rotary evaporator attached to a vacuum pump (3 mm Hg). Products obtained as solids or high boiling oils were dried under vacuum (1 mm Hg). Silica gel chromatography was performed either by CombiFlash® (Teledyne ISCO), SP4 or Isolera™ (Biotage) purification systems. All reactions were performed under a positive pressure of nitrogen, argon, or with a drying tube, at ambient temperature (unless otherwise stated), in anhydrous solvents, unless otherwise indicated.

Analytical thin-layer chromatography was performed on glass-backed Silica Gel 60_F 254 plates (Analtech, 0.25 mm) and eluted with the appropriate solvent ratios (v/v). The reactions were assayed by high performance liquid chromatography-mass spectrometry (LCMS) or thin-layer chromatography (TLC) and terminated as judged by the consumption of starting material. The TLC plates were visualized by UV, p-anisaldehyde, phosphomolybdic acid, or iodine staining. Microwave assisted reactions were run in a Biotage Initiator.

¹H NMR spectra were recorded on a Bruker XWIN-NMR (400 MHZ) spectrometer. Proton resonances are reported in parts per million (ppm) downfield from tetramethylsilane (TMS). ¹H NMR data are reported as multiplicity (s, singlet; d, doublet; t, triplet; q, quartet; quint, quintuplet; sept, septuplet; dd, doublet of doublets; ddd, means doublet of doublet of doublets; dt, doublet of triplets; td, means triplet of doublets; tt, means triplet of triplets; dq, means double quartet; qd, means quartet of doublets; bs, broad singlet). Exchangeable protons are not always observed. In NMR spectra, “br” means broad, “m” means multiplet, “J” means coupling constant. For spectra obtained in CDCl₃, DMSO-d₆, and CD₃OD, the residual protons (7.27, 2.50, and 3.31 ppm, respectively) were used as the internal reference. The progress of reactions and the purity of products were measured using the LCMS at 254 and 220 nm wavelengths and either electrospray ionization (ESI) positive mode or atmospheric-pressure chemical ionization (APCI) in positive mode.

### Purification Statement

All final compounds were purified to ≥95% purity. For most compounds, purity was determined by Agilent 1200 or 1260 Series HPLCs coupled to an Agilent 6120 or 6140 Quadrupole LC/MS with simultaneous UV (220 and 254 nm) and TIC detection (either APCI or ESI) or using a Shimadzu LC-20 HPLC with UV 220 nm detection or using an Agilent 1260 Infinity Hybrid HPLC/SFC with an Aurora A5 SFC Control Module, DAD, Leap PAL autosampler and 6120 single quadrupole MS detection. Chiral purity was analyzed using chiral SFC analysis with an Agilent 1260 Infinity Hybrid HPLC/SFC and by screening a variety of chiral columns.

### Abbreviations

Abs is absolute;
Ac is acetyl;
ACN or CH₃CN is acetonitrile;
AcOH is acetic acid;
approx. is approximately;
aq is aqueous;
ArX is aryl bromide or aryl chloride or aryl iodide;
Boc is tert-butyloxycarbonyl;
BPin is boronic acid pinacol ester;
B₂Pin₂ is bis (pinacolato)diboron;
° C. is degrees celsius;
cat is catalytic;
CDCl₃ is deutero-chloroform;
CN is cyano;
δ is chemical shift;
D₂O is deuterated water;
DABSO is 1,4-diazabicyclo[2.2.2]octane bis(sulfur dioxide) adduct;
DAST is diethylaminosulfur trifluoride;
DBU is 1,8-diazabicyclo[5.4.0]undec-7-ene;
DCC is N,N-dicyclohexylcarbodiimide;
DCM is dichloromethane; methylene chloride;
DIEA is N,N-diisopropylethylamine;
DHP is dihydro-2H-pyran;
DIAD is diisopropyl azodicarboxylate;
DIPEA is N-ethyldiisopropylamine, also known as N,N-diisopropylethylamine;
DMA is dimethylacetamide;
DMAD is dimethyl acetylenedicarboxylate;
DMAP is 4-(dimethylamino)pyridine;
DMB is 2,4-dimethoxybenzyl;
DMF is N,N-dimethylformamide;
DMSO is dimethyl sulfoxide;
DMSO-d₆ is deuterodimethylsulfoxide;
DPPA is diphenylphosphoryl azide;
Et is ethyl;
EtOAc is ethyl acetate;
EtOH is ethanol;
F/mol is faraday per mole;
g is gram;
HATU is 1-[bis(dimethylamino)methylene]-1H-1,2,3-triazolo[4,5-b]pyridinium 3-oxide hexafluorophosphate;
Het(Ar) and Ar(Het) as used in the Schemes means heteroaryl or aryl, as defined in the specification and claims;
HFIP is hexafluoroisopropanol;
HPLC is high pressure liquid chromatography;
HSPMB is 4-methoxy-a-toluenethiol;
hr(s) is hour(s);
L is liter;
LCMS is liquid chromatography mass spectrometry;
LDA is lithium diisopropylamide;
LiHMDS is lithium hexamethyldisilazide;
M is molar;
mA is milliampere or one thousandth of an ampere;
MeOH is methanol;
mg is milligram;
MHz is mega Hertz;
MIDA is N-methyliminodiacetic acid;
min(s) is minute(s);
mL is milliliter;
mmol is millimole;
mol is mole;
MPa is megapascal;
MS (m/z) is mass spectrum peak;
MsOH is methanesulfonic acid;
MTBE is tert-butyl methyl ether;
NaOtPn is sodium 2-methylbutan-2-olate;
nBuLi is n-butyl lithium;
n-Bu₂Mg is di-n-butylmagnesium;
NCS is N-chlorosuccinimide;
N/D is not determined;
NMR is nuclear magnetic resonance spectroscopy;
Pd/C is palladium on carbon;
Pd₂(dba)₃ is tris(dibenzylideneacetone)dipalladium(0);
Pd(dppf)Cl₂ is [1,1′-bis(diphenylphosphino)ferrocene]dichloropalladium(II);
Pd(OAc)₂ is palladium (II) acetate;
Pd(PPh₃)₄ is tetrakis(triphenylphosphine)palladium(0);
Pet. ether is petroleum ether and is the petroleum fraction consisting of aliphatic hydrocarbons and boiling in the range 35-60° C.;
PG is protecting group;
pH is power of hydrogen or potential of hydrogen;
Ph₃P is triphenylphosphine;
PMB is para-methoxybenzyl or 4-methoxybenzyl;
ppm is parts per million;
rt is room temperature;
R_{f} is retention factor;
Rₜ is retention time;
sat. is saturated;
SEM is 2-(trimethylsilyl)ethoxymethyl;
SEM-Cl is 2-(trimethylsilyl)ethoxymethyl chloride or [2-(chloromethoxy)ethyl](trimethyl)silane;
SFC is supercritical fluid chromatography;
SiO₂ is silica;
SM is starting material;
STAB is sodium triacetoxyborohydride;
tBu is tert-butyl;
TEA is triethylamine;
TFA is trifluoroacetic acid;
TFAA is trifluoroacetic anhydride;
THF is tetrahydrofuran;
THP is tetrahydropyran;
TLC is thin layer chromatography;
TMPLi is tetramethylpiperidine lithium;
TMS is trimethylsilyl and tetramethylsilane;
Turbo grignard is isopropylmagnesium chloride lithium chloride complex solution;
μL is microliter;
μmol is micromole;
Xantphos is 4,5-bis(diphenylphosphino)-9,9-dimethylxanthene; and
XPhos is 2-dicyclohexylphosphino-2′,4′,6′-triisopropylbiphenyl.

The Schemes described below are intended to provide a general description of the methodology employed in the preparation of the compounds of the present invention. Some of the compounds of the present invention contain one or two chiral centers. In the following Schemes, the general methods for the preparation of the compounds are shown either in racemic or enantioenriched form. It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### General Methods

Unless stated otherwise, the variables in Schemes I-X have the same meanings as defined herein.

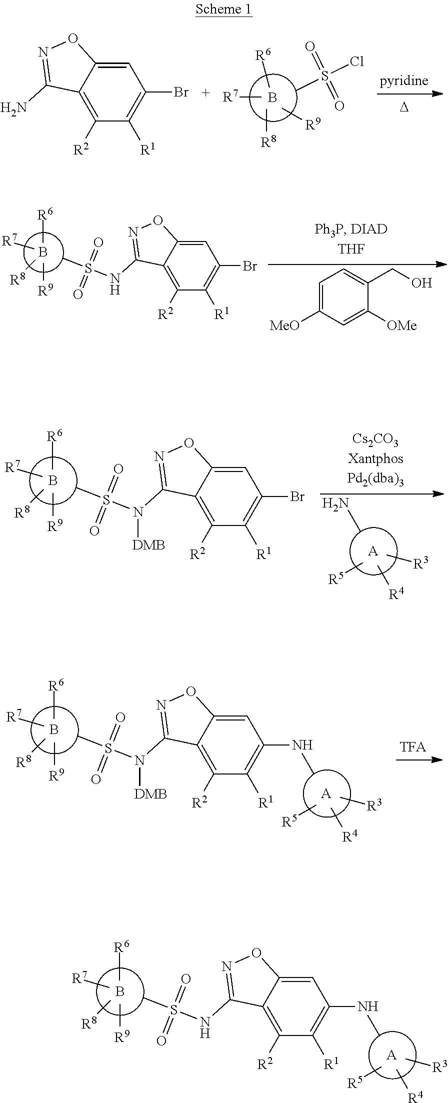

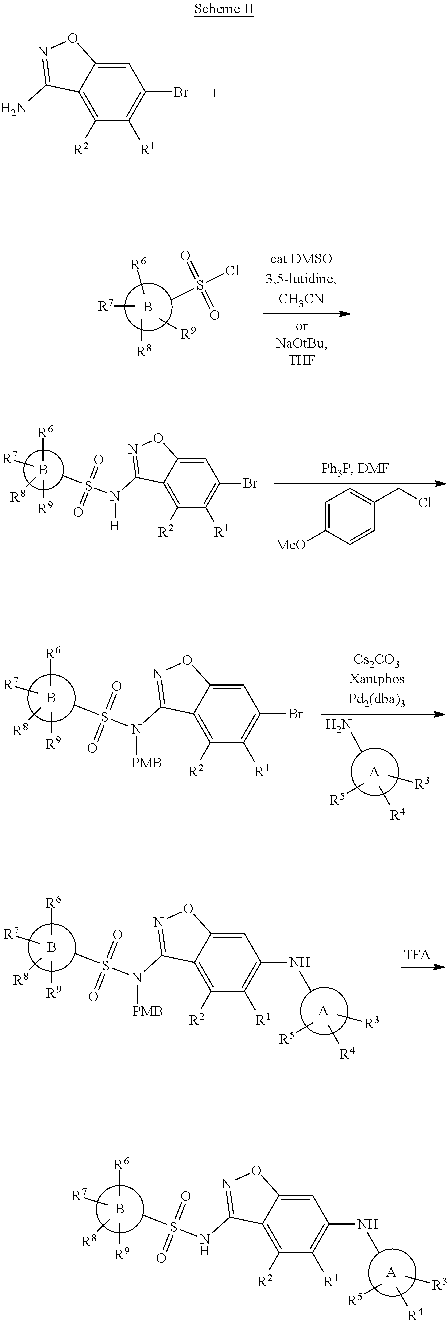

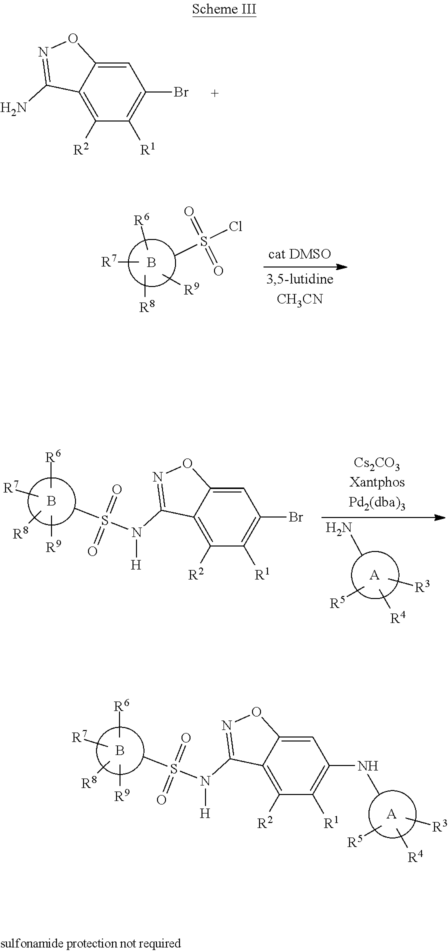

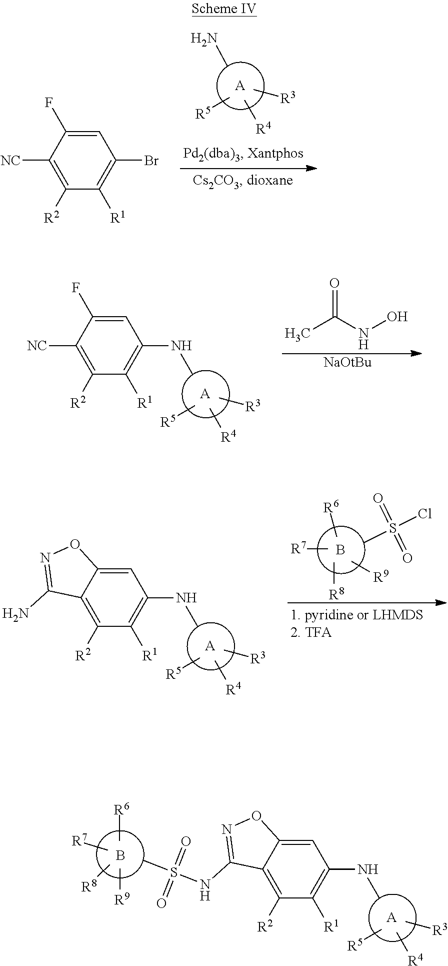

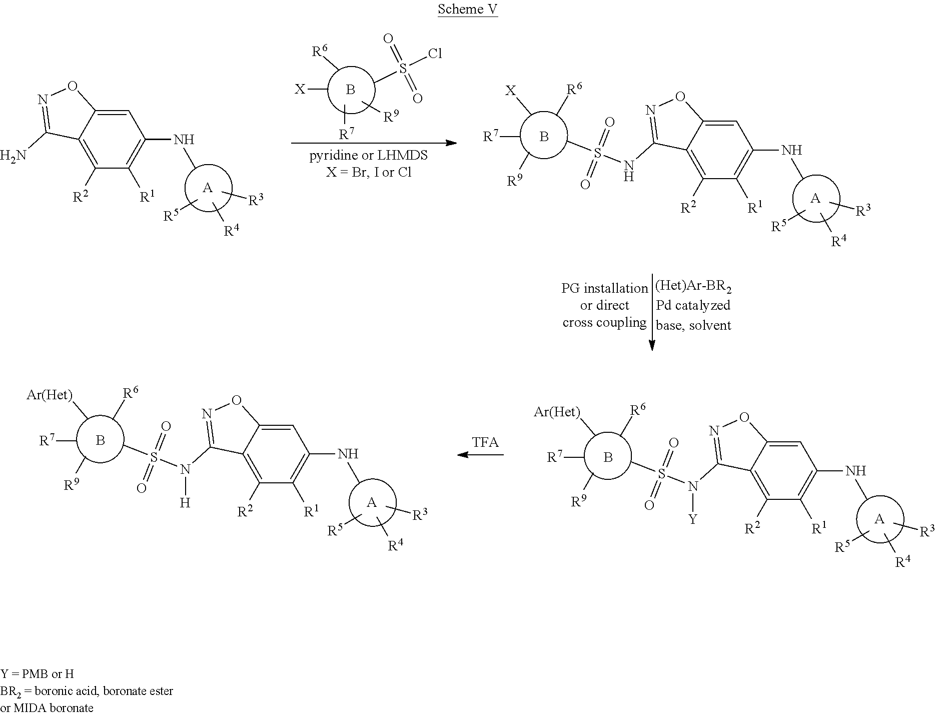

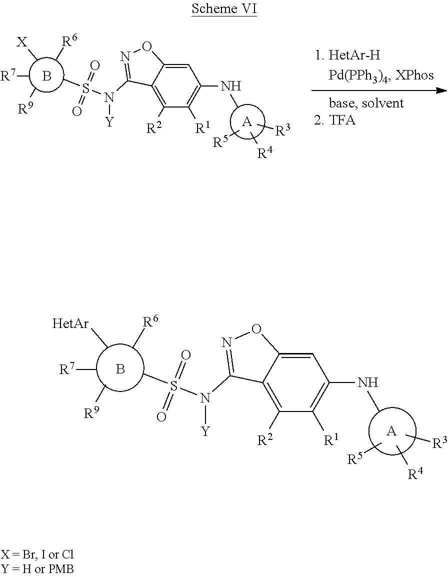

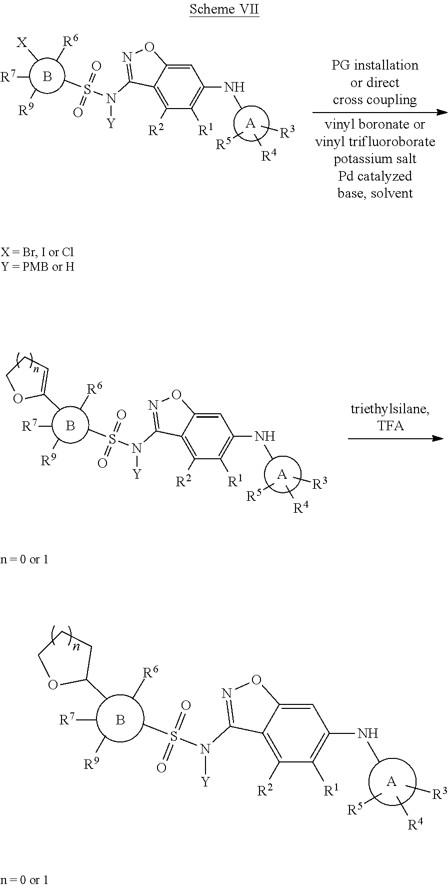

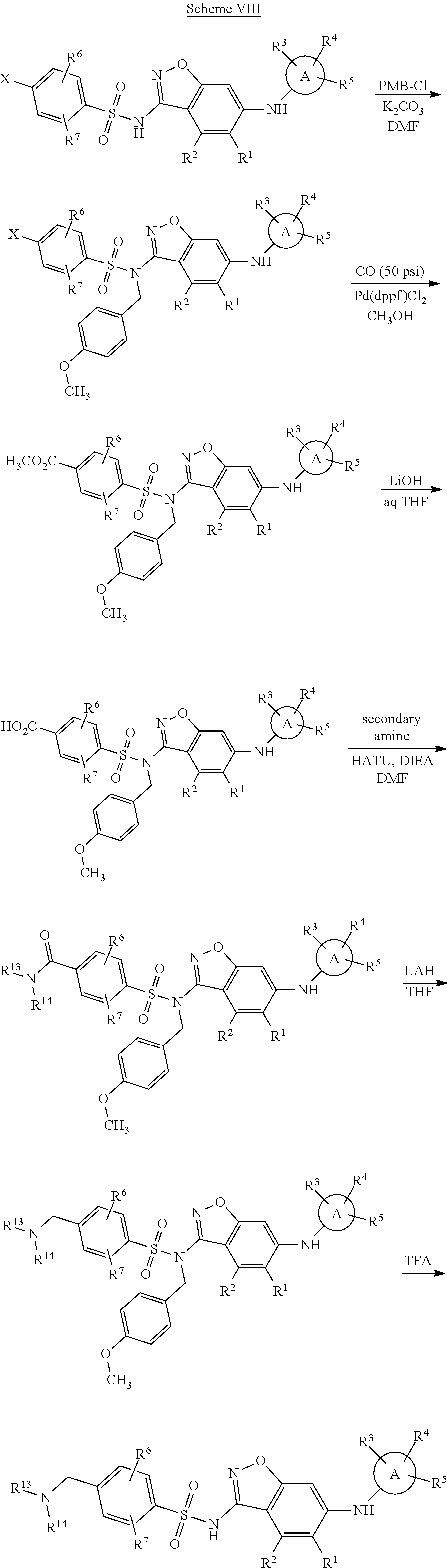

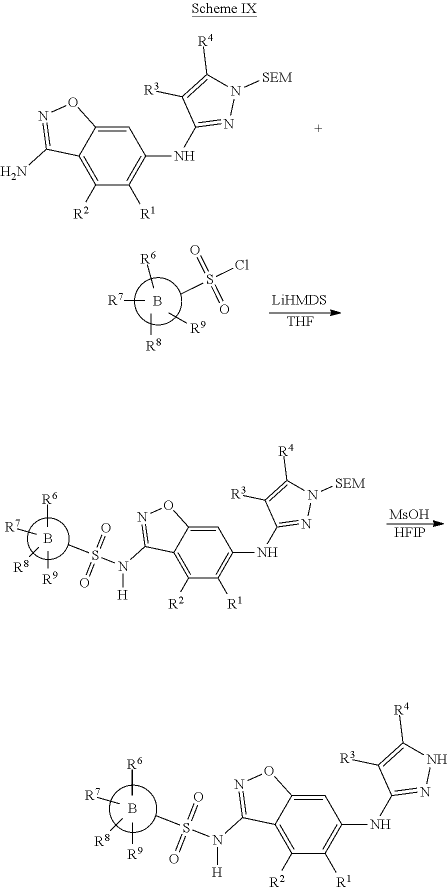

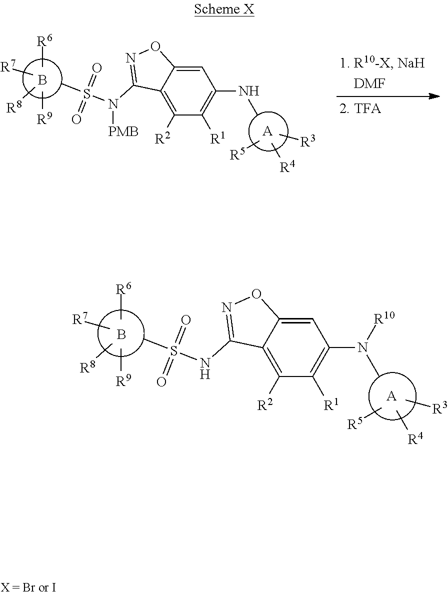

In some cases, compounds described in Schemes I-VIII may contain protecting groups, which may be appended or removed by additional steps in the synthetic sequence using conditions known in the art (March's Advanced Organic Chemistry: Reactions, Mechanisms, and Structure 8th Edition or Protecting Groups, 10 Georg Thieme Verlag, 1994). Compounds at every step may be purified by standard techniques, such as column chromatography, crystallization, or reverse phase SFC or HPLC. Ring A, Ring B, R¹, R², R³, R⁴, R⁵, R⁶, R⁷, R⁸, R⁹ R¹⁰, R¹³ and R¹⁴ are as defined in the embodiments, schemes, examples, and claims herein.

### EXAMPLES

In order that this invention may be better understood, the following examples are set forth. These examples are for purposes of illustration only and are not to be construed as limiting the scope of the invention in any manner.

The compounds and intermediates described below were named using the naming convention provided with ACD/Spectrus Processor 2019.1.1, Version S05S41; 2020.2.1.1, Version C25H41; or 2022.2.0, Version C45H41 (Advanced Chemistry Development, 8 King Street East, Suite 107, Toronto, Ontario, M5C 1B5, Canada). The naming convention provided with ACD/Spectrus Processor 2019.1.1, Version S05S41; 2020.2.1.1, Version C25H41; or 2022.2.0, Version C45H41, is well known by those skilled in the art and it is believed that the naming convention provided with ACD/Spectrus Processor 2019.1.1, Version S05S41; 2020.2.1.1, Version C25H41; or 2022.2.0, Version C45H41, generally comports with the IUPAC (International Union for Pure and Applied Chemistry) recommendations on Nomenclature of Organic Chemistry and the CAS Index rules. Unless noted otherwise, all reactants were obtained commercially without further purifications or were prepared using methods known in the literature.

### Preparations of Synthetic Intermediates

### Intermediate A: 6-Bromo-4-methoxy-1,2-benzoxazol-3-amine

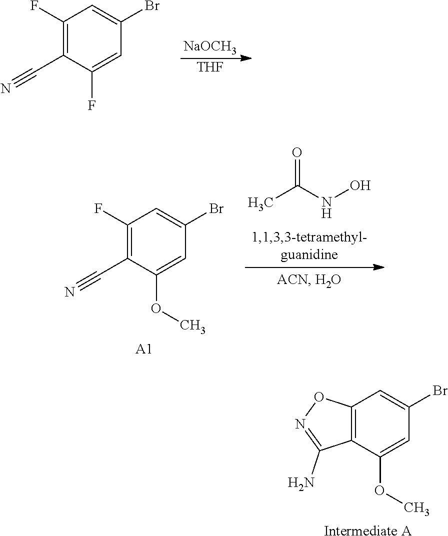

4-Bromo-2-fluoro-6-methoxybenzonitrile (A1): To a cooled (0° C.) solution of 4-bromo-2,6-difluorobenzonitrile (100 g, 459 mmol) in THF (1000 mL) was added sodium methoxide (65 g, 360 mmol) in several portions. When the addition was complete, stirring was continued at rt (30° C.) for 16 hrs. The mixture was quenched with H₂O (600 mL) and extracted with EtOAc (2×400 mL). The combined organic extracts were concentrated under reduced pressure, then the residue was recrystallized from DCM (100 mL) and Pet. ether (300 mL) to give A1 (64.5 g, 61%) as a white solid. ¹H NMR (400 MHZ, CHLOROFORM-d) δ 7.10-6.99 (m, 1H), 6.99-6.93 (m, 1H), 3.98 (br s, 3H).

6-Bromo-4-methoxy-1,2-benzoxazol-3-amine (Intermediate A): A mixture of 4-bromo-2-fluoro-6-methoxybenzonitrile (A1) (26.0 g, 90 mmol), N-hydroxyacetamide (20.4 g, 271 mmol), and 1,1,3,3-tetramethylguanidine (62.5 g, 543 mmol) in ACN (240 mL) and H₂O (30 mL) was heated at 60° C. for 16 hrs. After cooling to rt, the solvent was removed under vacuum. H₂O (50 mL) was added to the residue, causing a white precipitate to form. The precipitate was collected by filtration and dried to give Intermediate A (17.0 g, 77% yield) as a white solid. ¹H NMR (400 MHz, DMSO-d₆) δ7.32 (d, J=0.9 Hz, 1H), 6.91 (s, 1H), 6.05 (s, 2H), 3.93 (s, 3H).

### Intermediate B: 6-Bromo-5-methoxy-1,2-benzoxazol-3-amine

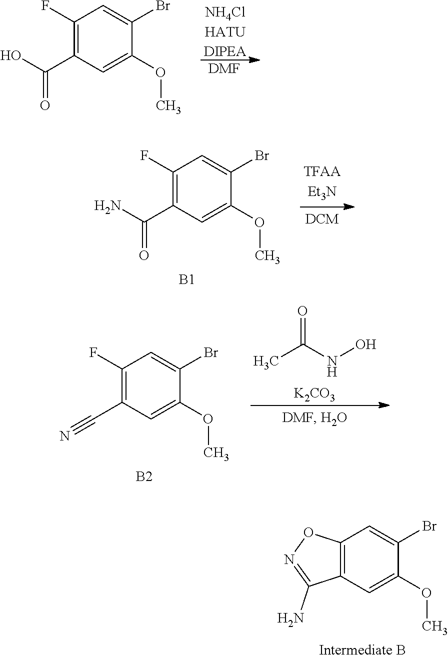

4-Bromo-2-fluoro-5-methoxybenzamide (B1): DIPEA (17.1 g, 133 mmol) was added to a solution of 4-bromo-2-fluoro-5-methoxybenzoic acid (11.0 g, 44.2 mmol) and ammonium chloride (3.58 g, 66.9 mmol) in DMF (100 mL) at rt (15° C.). Next, HATU (25.2 g, 66.3 mmol) was added in portions, slowly enough to maintain the internal temperature below 20° C. When the addition was complete, stirring was continued at rt (15° C.) for two hrs. The reaction mixture was poured into cold H₂O (200 mL) with stirring, causing a precipitate to form. The solids were collected by filtration, washed with H₂O, and dried to give B1 (9.3 g, 85%) as a white solid. ¹H NMR (400 MHZ, DMSO-d₆) δ 7.75 (br s, 2H), 7.67 (d, J=9.5 Hz, 1H), 7.31 (d, J=6.2 Hz, 1H), 3.87 (s, 3H).

4-Bromo-2-fluoro-5-methoxybenzonitrile (B2): A suspension of 4-bromo-2-fluoro-5-methoxybenzamide (B1) (9.3 g, 37.5 mmol) and TEA (11.4 g, 112 mmol) in DCM (180 mL) at 15° C. was treated dropwise with TFAA (11.8 g, 56 mmol). The resulting clear solution was stirred at 15° C. for two hrs. The mixture was poured into H₂O (200 mL), the layers separated, and the aqueous layer extracted with DCM (100 mL). The combined organic layers were washed with brine, dried over sodium sulfate, and concentrated to give B2 (10 g, >100%) as a yellow solid. ¹H NMR (400 MHZ, CHLOROFORM-d) δ 7.48 (d, J=7.8 Hz, 1H), 7.04 (d, J=5.3 Hz, 1H), 3.92 (s, 3H).

6-Bromo-5-methoxy-1,2-benzoxazol-3-amine (Intermediate B): 4-Bromo-2-fluoro-5-methoxybenzonitrile (B2) (6.00 g, 23 mmol) and potassium carbonate (18.8 g, 136 mmol) were suspended in DMF (140 mL) and H₂O (20 mL) at rt (20° C.). N-hydroxyacetamide (5.11 g, 68 mmol) was added and the mixture heated at 60° C. for three hrs. After cooling to rt, the crude reaction mixture was poured into ice water (250 mL), and the resulting precipitate was collected by filtration. The filter cake was washed with H₂O (50 mL) and dried, to give a first crop of product. The aqueous filtrate was extracted with EtOAc (2×100 mL). The combined organic extracts were washed with brine (3×50 mL), dried over sodium sulfate, concentrated, and the residue (900 mg) recrystallized from 20 mL of 1:4 EtOAc/Pet. ether, affording a second crop of product. The two product crops were combined and dried under vacuum to give Intermediate B (4.7 g, 85%) as a light-yellow solid. LCMS m/z 242.8 [M+H]⁺; ¹H NMR (400 MHZ, DMSO-d₆) δ 7.83 (s, 1H), 7.52 (s, 1H), 6.40 (s, 2H), 3.86 (s, 3H).

### Intermediate C: 6-Bromo-5-fluoro-4-methoxy-1,2-benzoxazol-3-amine

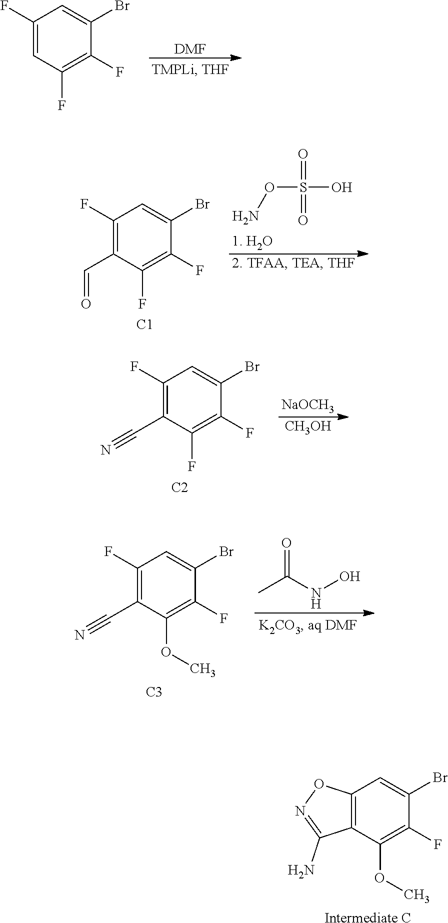

4-Bromo-2,3,6-trifluorobenzaldehyde (C1): To a solution of 1-bromo-2,3,5-trifluorobenzene (200 g, 948 mmol, 114 mL) in THF (2000 mL) was added dropwise TMPLi (1 M, 1422 mmol, 1.42 L) at −65° C. and stirred at 25° C. for one hour. Then DMF (83.2 g, 1.14 mol, 87.5 mL) was added dropwise at 25° C. and the crude reaction mixture was stirred at 25° C. for one hour. LCMS showed the starting material was consumed completely and desired product was formed. The process was repeated and the two batches were combined. The reaction was quenched with saturated NH₄Cl (2000 mL) at 0˜5° C. and adjusted to pH=5 with 2 N aqueous HCl. The aqueous layer was extracted with EtOAc (2000 mL×2). The combined organic layers were washed with brine (2000 mL), dried over MgSO₄, filtered and the filtrate was concentrated. The crude product was purified by silica gel chromatography and eluted with Pet. ether-THF (1:0˜97:3) and gave C1 as a yellow solid (158 g, 34%). ¹H NMR (400 MHZ, CHLOROFORM-d) δ 10.21 (s, 1H), 7.22-7.19 (m, 1H).

4-Bromo-2,3,6-trifluorobenzonitrile (C2): To a solution of 4-bromo-2,3,6-trifluorobenzaldehyde (C1) (108 g, 452 mmol) in H₂O (1000 mL) was added hydroxylamine-O-sulfonic acid (66.4 g, 588 mmol) and then the reaction was stirred at 105° C. for four hrs. TLC (Pet. ether-EtOAc=10:1, Rₜ=0.5) showed the starting material was consumed completely. The reaction was extracted with EtOAc (1000 mL×2). The combined organic layers were washed with brine (1000 mL), dried over MgSO₄, filtered and the filtrate was concentrated to the crude product. To a solution of the crude product (114 g, 449 mmol) and TEA (136.2 g, 1.4 mol, 187 mL) in THF (1200 mL) was added TFAA (113.1 g, 539 mmol, 74.9 mL) at 0˜5° C. and then the reaction was stirred at 25° C. for 2 hrs. TLC (Pet. ether-EtOAc=10:1, R_{f}=0.4) showed the starting material was consumed completely. The reaction was added to H₂O (1000 mL) and the aqueous layer was extracted with EtOAc (1000 mL×2). The combined organic layers were washed with brine (1000 mL), dried over MgSO₄, filtered and the filtrate was concentrated to the crude product. The crude product was combined with the crude product from a previous experiment (50 g, 209 mmol) of 4-bromo-2,3,6-trifluorobenzaldehyde (C1) and purified by silica gel chromatography and eluted with Pet. ether-THF (1:0˜98:2) and gave C2 as a yellow solid (115 g, 74% yield). ¹H NMR (400 MHZ, DMSO-d₆) δ 8.11-8.07 (m, 1H). 4-Bromo-3,6-difluoro-2-methoxybenzonitrile (C3): To a solution of 4-bromo-2,3,6-trifluorobenzonitrile (C2) (94.3 g, 400 mmol) in methanol (1000 mL) was added dropwise sodium methoxide (72 g, 400 mmol) at 0˜5° C. and then the reaction was stirred at 0˜5° C. for one hour. LCMS showed the starting material was consumed completely. The reaction was added to H₂O (1000 mL) and the aqueous layer was extracted with EtOAc (1000 mL×2). The combined organic layers were washed with brine (1000 mL), dried over MgSO₄, filtered and the filtrate was concentrated to give the crude product. The crude product was purified by silica gel chromatography and eluted with Pet. ether-THF (1:0˜99:1) and gave C3 as a yellow solid (84.3 g, 85%). ¹H NMR (400 MHZ, DMSO-d₆) δ 7.78 (dd, J=8.5, 4.9 Hz, 1H), 4.16 (d, J=3.4 Hz, 3H).

6-Bromo-5-fluoro-4-methoxy-1,2-benzoxazol-3-amine (Intermediate C): To a solution of 4-bromo-3,6-difluoro-2-methoxybenzonitrile (C3) (84.3 g, 340 mmol), N-hydroxyacetamide (76.5 g, 1.02 mol) in DMF (800 mL) and H₂O (100 mL) was added K₂CO₃ (281.8 g, 2.04 mol) and then the reaction was stirred at 60° C. for two hrs. LCMS showed the starting material was consumed completely and the desired product was formed. The reaction was added to H₂O (1000 mL) and extracted with EtOAc (1000 mL×2). The combined organic layers were washed with brine (1000 mL), dried over MgSO₄, filtered and the filtrate was concentrated to the crude product. The crude product was triturated with Pet. ether-EtOAc=10:1 (250 mL) for one hr at 25° C. The mixture was filtered and the filter cake washed with Pet. ether (20 mL×2). The cake was collected and concentrated under reduced pressure and gave Intermediate C as an off-white solid (53.5 g, 60%). LCMS m/z 261/263 [M+H]⁺; ¹H NMR (400 MHZ, DMSO-d⁶) δ 7.58 (d, J=4.2 Hz, 1H), 6.19 (s, 2H), 4.10 (d, J=3.8 Hz, 3H).

### Intermediate D: 3-Methoxy-5,6,7,8-tetrahydronaphthalene-2-sulfonyl chloride

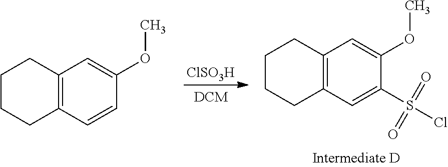

3-Methoxy-5,6,7,8-tetrahydronaphthalene-2-sulfonyl chloride (Intermediate D): Chlorosulfonic acid (338 mg, 0.22 mL, 3.33 mmol) was added dropwise to a cooled (0° C.) solution of 6-methoxy-1,2,3,4-tetrahydronaphthalene (200 mg, 1.1 mmol) in DCM (3.0 mL) under nitrogen. The solution was stirred at 0° C. for 1 hr, during which the color changed from grey to purple. The mixture was slowly poured into ice water and extracted with DCM (2×30 mL). The combined organic extracts were washed with H₂O (2×30 mL) and brine (30 mL), dried over sodium sulfate, filtered, and concentrated to give Intermediate D (230 mg, 80%) as a white solid. ¹H NMR (400 MHZ, CHLOROFORM-d) δ 7.65 (s, 1H), 6.79 (s, 1H), 4.01 (s, 3H), 2.84 (br s, 2H), 2.75 (br s, 2H), 1.82 (td, J=3.4, 6.4 Hz, 4H).

The sulfonyl chloride intermediates in Table 14 were prepared according to the general method of Intermediate D.

TABLE 14

| Intermediate | | |
|---|---|---|
| Designation | Structure/IUPAC name | Analytical Data |
| Da | 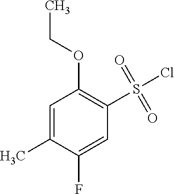 | ¹H NMR (400 MHz, CDCl₃) d 7.62 (δ, J = 8.3 Hz, 1H), 6.91 (d, J = 5.8 Hz, 1H), 4.24 (q, J = 7.0 Hz, 2H), 2.38 (d, J = 2.0 Hz, 3H), 1.55 (t, J = 7.0 Hz, 3H) |
| | 2-ethoxy-5-fluoro-4- | |
| | methylbenzene-1-sulfonyl chloride | |
| Db | 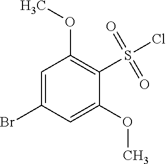 | ¹H NMR (400 MHz, CDCl₃) δ 6.83 (s, 2 H) 3.99 (s, 6 H) |
| | 4-bromo-2,6-dimethoxybenzene-1- | |
| | sulfonyl chloride | |

### Intermediate E: 2-Ethoxy-4-methylbenzene-1-sulfonyl chloride

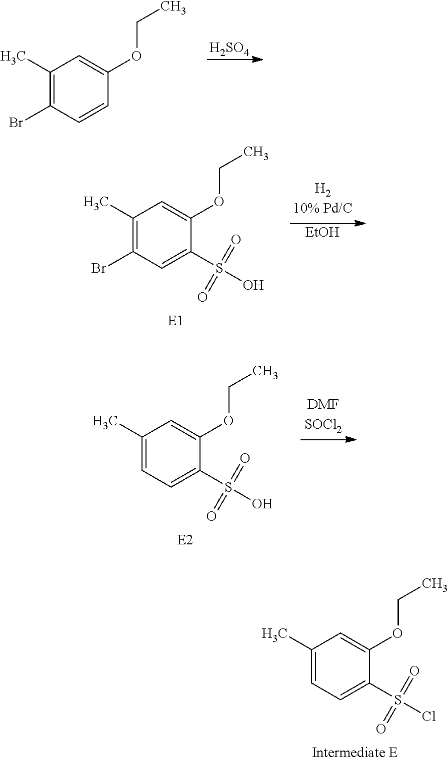

5-Bromo-2-ethoxy-4-methylbenzene-1-sulfonic acid (E1): To an ice bath-cooled flask containing concentrated sulfuric acid (300 mL) was added 1-bromo-4-ethoxy-2-methylbenzene (158 g, 732 mmol) dropwise over 10 mins. The resulting light brown mixture was stirred overnight, then quenched by adding crushed ice (750 mL), causing a white precipitate to form. The solid was collected by filtration and dried to give E1 (180 g, 83%) as a white solid. 2-Ethoxy-4-methylbenzene-1-sulfonic acid (E2): A suspension of 5-bromo-2-ethoxy-4-methylbenzene-1-sulfonic acid (E1) (150 g, 508 mmol) and 10% Pd/C (27 g) in ethanol (1000 mL) was reacted under 0.4 MPa hydrogen at 60° C. overnight. The mixture was filtered through a powdered cellulose pad to remove solids, and the filtrate concentrated to dryness, leaving E2 (96.6 g, 88%) as a white solid.

2-Ethoxy-4-methylbenzene-1-sulfonyl chloride (Intermediate E): A flask containing 2-ethoxy-4-methylbenzene-1-sulfonic acid (E2) (46.2 g, 214 mmol) was cooled in an ice bath. Thionyl chloride (240 mL, 3.3 mol) and DMF (30 drops) were added over 30 mins. The mixture was heated at reflux for two hrs, then allowed to stir at rt overnight. The mixture was concentrated to dryness, leaving Intermediate E (33.6 g, 67%) as a white solid. GCMS m/z 234 M⁺; ¹H NMR (400 MHZ, CHLOROFORM-d) δ 7.81 (d, J=8.5 Hz, 1H), 6.87-6.85 (m. 2H), 4.25 (q, J=7.0 Hz, 2H), 2.44 (s, 3H), 1.54 (t, J=7.0 Hz, 3H).

### Intermediate F: 2-Ethoxy-6-methoxybenzene-1-sulfonyl chloride

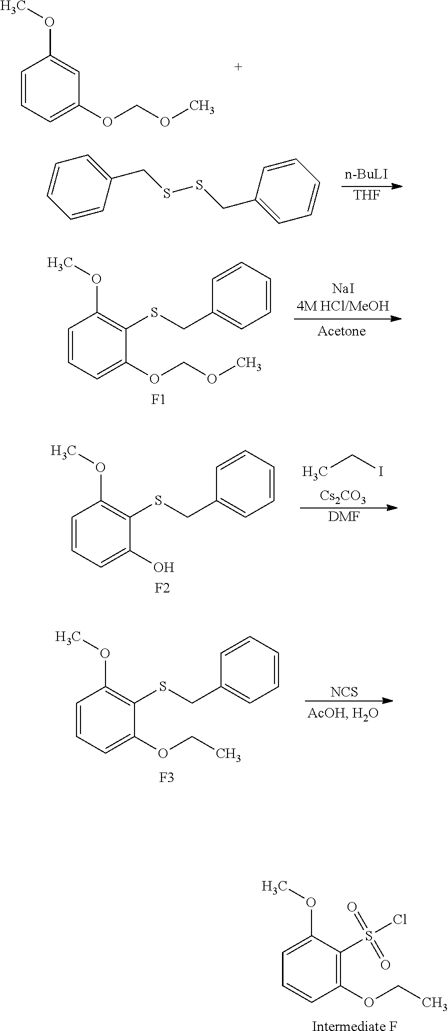

2-(Benzylsulfanyl)-1-methoxy-3-(methoxymethoxy)benzene (F1): To a cooled (0° C.) solution of 1-methoxy-3-(methoxymethoxy)benzene (6.00 g, 35.7 mmol) in dry THF (150 mL) was added n-butyllithium (2.5 M in THF, 17.8 mL, 44.6 mmol) dropwise under a dry nitrogen atmosphere. After the addition was complete, stirring was continued for one hr at 0° C., then dibenzyldisulfide (10.5 g, 42.8 mmol) was added in portions. Stirring was continued at 0° C. for two hrs, then at rt overnight. The reaction was quenched with methanol, adsorbed directly onto silica gel, and purified by silica gel chromatography (eluting with 0-50% EtOAc in Pet. ether) to give F1 (7.7 g, 74%) as a yellow oil. ¹H NMR (400 MHZ, CHLOROFORM-d) δ 7.24-7.14 (m, 6H), 6.77 (d, J=8.3 Hz, 1H), 6.60 (d, J=8.3 Hz, 1H), 5.09 (s, 2H), 4.02 (s, 2H), 3.85 (s, 3H), 3.44 (s, 3H).

2-(Benzylsulfanyl)-3-methoxyphenol (F2): A solution of 2-(benzylsulfanyl)-1-methoxy-3-(methoxymethoxy)benzene (F1) (7.70 g, 26.5 mmol) in acetone (330 mL) was treated with sodium iodide (4.77 g, 31.8 mmol) and stirred until all dissolved. The solution was cooled to 0° C., then a solution of HCl in methanol (4.0 M in dioxane, 166 mL, 664 mmol) was added dropwise. The cloudy yellow mixture was stirred at 0° C. for 10 mins, then poured into saturated aq NaHCO₃ (500 mL). Solid NaHCO₃ was added until the pH=7. The mixture was extracted with EtOAc (300 mL). The organic layer was washed with brine (200 mL), dried over sodium sulfate, and concentrated to afford crude F2 (7.0 g, 100%) as a yellow oil. ¹H NMR (400 MHZ, CHLOROFORM-d) δ 7.30-7.15 (m, 4H), 7.15-7.08 (m, 2H), 6.74 (s, 1H), 6.57 (dd, J=1.1, 8.3 Hz, 1H), 6.47 (d, J=8.3 Hz, 1H), 3.88 (s, 2H), 3.87 (s, 3H). 2-(Benzylsulfanyl)-1-ethoxy-3-methoxybenzene (F3): A solution of 2-(benzylsulfanyl)-3-methoxyphenol (F2) (1.0 g, 3.78 mmol), iodoethane (807 mg, 5.18 mmol), and cesium carbonate (2.81 g, 8.63 mmol) in dry DMF (20.0 mL) was stirred at 80° C. for 16 hrs. The mixture was poured into brine (30 mL) and extracted with EtOAc (2×50 mL). The combined organic extracts were washed with brine (30 mL), dried over sodium sulfate, concentrated, and purified by silica gel chromatography (eluting with 0-50% EtOAc in Pet. ether), yielding F3 (900 mg, 86%) as a pale-yellow oil. LCMS m/z 275 [M+H]⁺; ¹H NMR (400 MHZ, CHLOROFORM-d) δ 7.26-7.10 (m, 6H), 6.52 (d, J=8.4 Hz, 2H), 4.07-3.98 (m, 4H), 3.85-3.77 (m, 3H), 1.44 (t, J=6.9 Hz, 3H).

2-Ethoxy-6-methoxybenzene-1-sulfonyl chloride (Intermediate F): To a cooled (0° C.) solution of 2-(benzylsulfanyl)-1-ethoxy-3-methoxybenzene (F3) (900 mg, 3.28 mmol) in acetic acid (27 mL) and H₂O (9.0 mL) was added N-chlorosuccinimide (482 mg, 3.61 mmol). Stirring was continued at 0° C. for a few mins, then a second portion of NCS (482 mg, 3.61 mmol) was added, and the mixture stirred at 20° C. for 30 mins, but no further conversion was observed. A third portion of NCS (482 mg, 3.61 mmol) was added and stirring continued at 15° C. for 2.5 hrs. The reaction mixture was poured into saturated aq NaHCO₃ (100 mL), and solid NaHCO₃ was carefully added to basify the solution. The mixture was extracted with EtOAc (2×50 mL). The combined organic extracts were washed with brine, dried over sodium sulfate, concentrated, and immediately purified by silica gel chromatography (eluting with 0-50% EtOAc in Pet. ether), yielding Intermediate F (700 mg, 85%) as a colorless oil which solidified on standing. The product was stored at 0° C. to retard decomposition. ¹H NMR (400 MHZ, CHLOROFORM-d) δ 7.53 (t, J=8.5 Hz, 1H), 6.65 (d, J=8.5 Hz, 2H), 4.73 (s, 1H), 4.22 (q, J=7.0 Hz, 2H), 3.99 (s, 3H), 1.54 (t, J=7.0 Hz, 3H).

The sulfonyl chloride intermediates in Table 15 were prepared according to the general method of Intermediate F., using the indicated alkyl halide instead of iodoethane in the ether formation step.

TABLE 15

| Intermediate | | | Alkyl halide |
|---|---|---|---|
| Designation | Structure/IUPAC name | Analytical Data | used |
| Fa | 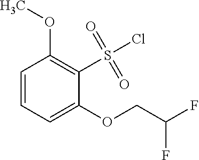 [SMILES: COc1cccc(OCC(F)F)c1S(=O)(=O)Cl] | ¹H NMR (400 MHz, CHLOROFORM-d) δ 7.58 (t, J = 8.5 Hz, 1H), 6.77 (d, J = 8.3 Hz, 1H), 6.64 (d, J = 8.0 Hz, 1H), 6.22 (tt, J = 4.0, 55.0 Hz, 1H), 4.33 (dt, J = 4.1, 12.5 Hz, 2H), 4.01 (s, 3H) | 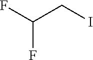 1,1-difluoro- 2-iodoethane |
| | 2-(2,2-difluoroethoxy)-6- | | |
| | methoxybenzene-1- | | |
| | sulfonyl chloride | | |
| Fb | 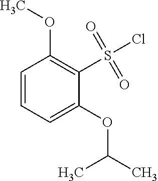 [SMILES: COc1cccc(OC(C)C)c1S(=O)(=O)Cl] | ¹H NMR (400 MHz, CHLOROFORM-d) δ 7.42 (t, J = 8.5 Hz, 1H), 6.57 (d, J = 8.1 Hz, 1H), 6.52 (d, J = 8.5 Hz, 1H), 4.70- 4.59 (m, 1H), 3.89 (s, 3H), 1.37 (d, J = 6.0 Hz, 6H) | 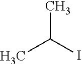 2-iodo- propane |
| | 2-methoxy-6-[(propan-2- | | |
| | yl)oxy]benzene-1-sulfonyl | | |
| | chloride | | |
| Fc | 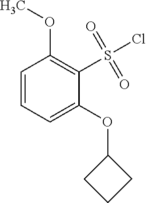 [SMILES: COc1cccc(OC2CCC2)c1S(=O)(=O)Cl] | ¹H NMR (400 MHz, CHLOROFORM-d) δ 7.48 (t, J = 8.5 Hz, 1H), 6.62 (d, J = 8.5 Hz, 1H), 6.47 (d, J = 8.5 Hz, 1H), 4.78 (quin, J = 7.1 Hz, 1H), 3.97 (s, 3H), 2.55-2.44 (m, 2H), 2.41-2.27 (m, 2H), 1.99-1.88 (m, 1H), 1.79- 1.65 (m, 1H) | 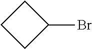 bromo- cyclobutane |
| | 2-(cyclobutyloxy)-6- | | |
| | methoxybenzene-1- | | |
| | sulfonyl chloride | | |

### Intermediate G: 2-(2,2-Difluoroethoxy)-4-methylbenzene-1-sulfonyl chloride

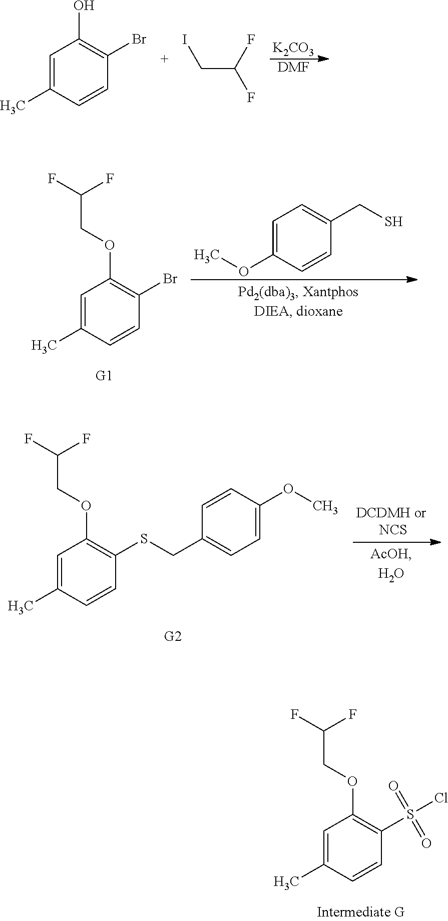

1-Bromo-2-(2,2-difluoroethoxy)-4-methylbenzene (G1): A mixture of 2-bromo-5-methylphenol (1.0 g, 5.3 mmol), 1,1-difluoro-2-iodoethane (2.0 g, 10.7 mmol), and potassium carbonate (2.2 g, 16 mmol) in DMF (10 mL) was heated at 80° C. for 16 hrs. The mixture was diluted with H₂O (100 mL) and extracted with EtOAc (100 mL). The organic layer was washed with brine, dried over Na₂SO₄, filtered and concentrated to provide G1 as a brown solid (1.4 g, quantitative). TLC (1/4 Pet. ether/EtOAc) R_{f} 0.3.

2-(2,2-Difluoroethoxy)-1-{[(4-methoxyphenyl)methyl]sulfanyl}-4-methylbenzene (G2): A solution of 1-bromo-2-(2,2-difluoroethoxy)-4-methylbenzene (G1) (700 mg, 2.8 mmol), Pd₂(dba)₃ (102 mg, 0.11 mmol), Xantphos (129 mg, 0.22 mmol), and (4-methoxyphenyl) methanethiol (1.3 g, 8.4 mmol) in dioxane (16 mL) was degassed and purged with nitrogen three times, then diisopropylamine (2.4 mL, 13.9 mmol) was added. The crude reaction mixture was heated at 105° C. for 16 hrs. The crude reaction mixture was concentrated under reduced pressure and the residue was purified using silica gel chromatography and eluted with 0-40% EtOAc-Pet. ether which gave G2 as a white solid (400 mg, 44%). TLC (1/4 Pet. ether/EtOAc) R_{f} 0.2; ¹H NMR (400 MHZ, CHLOROFORM-d) δ 7.24-7.15 (m, 3H), 6.85-6.75 (m, 3H), 6.69 (s, 1H), 6.35-5.96 (m, 1H), 4.23 (dt, J=4.3, 13.1 Hz, 2H), 4.04 (s, 2H), 3.80 (s, 3H), 2.42-2.26 (m, 3H).

2-(2,2-Difluoroethoxy)-4-methylbenzene-1-sulfonyl chloride (Intermediate G): To a solution of 2-(2,2-difluoroethoxy)-1-{[(4-methoxyphenyl)methyl]sulfanyl}-4-methylbenzene (G2) (400 mg, 1.2 mmol) in ACN (16 mL) was added acetic acid (0.7 mL), H₂O (0.5 mL) and 1,3-dichloro-5,5-dimethylhydantoin (486 mg, 2.5 mmol). The later was added in portions at 0° C. After the addition, the crude reaction mixture was stirred at 0-10° C. for 1 hour. The crude reaction mixture was diluted with EtOAc (20 mL) and washed with aq sodium bicarbonate (5 mL), brine (5 mL), dried over Na₂SO₄, filtered and concentrated. The crude product was purified by silica gel chromatography, eluted with 0-40% EtOAc-Pet. ether and gave Intermediate G as a white solid (250 mg, 75%). TLC (1/2 EtOAc/Pet. ether) R_{f} 0.1 vs R_{f} 0.3 for starting material; ¹H NMR (400 MHZ, CHLOROFORM-d) δ 7.88 (d, J=8.3 Hz, 1H), 7.01 (d, J=8.3 Hz, 1H), 6.92 (s, 1H), 6.38-6.08 (m, 1H), 4.41 (dt, J=4.3, 12.4 Hz, 2H), 2.49 (s, 3H).

The sulfonyl chloride intermediates in Table 16 were prepared according to the general method of Intermediate G.

TABLE 16

| Intermediate | | |
|---|---|---|
| Designation | Structure/IUPAC name | Analytical Data |
| Ga | 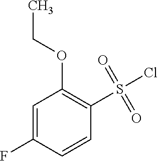 | ¹H NMR (400 MHz, CHLOROFORM-d) δ 8.02-7.95 (m, 1H), 6.84- 6.75 (m, 2H), 4.27 (q, J = 7.0 Hz, 2H), 1.58 (t, J = 7.0 Hz, 3H) |
| | 2-ethoxy-4-fluorobenzene-1- | |
| | sulfonyl chloride | |
| Gb | 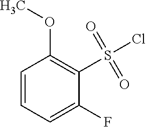 | ¹H NMR (400 MHz, CHLOROFORM-d) δ 7.65-7.60 (m, 1H), 6.94-6.82 (m, 2H), 4.06 (s, 3H) |
| | 2-fluoro-6-methoxybenzene-1- | |
| | sulfonyl chloride | |
| Gc | 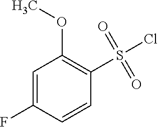 | ¹H NMR (400 MHz, CHLOROFORM-d) δ 8.00 (dd, J = 8.7, 5.9 Hz, 1H), 6.90 (d, J = 8.6 Hz, 1H), 6.85-6.82 (m, 1H) 4.07 (s, 3H) |
| | 4-fluoro-2-methoxybenzene-1- | |
| | sulfonyl chloride | |
| Gd | 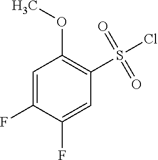 | ¹H NMR (400 MHz, CHLOROFORM-d) δ 7.87 (t, J = 8.7 Hz, 1H), 6.96 (dd, J = 11.3, 5.9 Hz, 1H), 4.05 (s, 3H) |
| | 4,5-difluoro-2-methoxybenzene-1- | |
| | sulfonyl chloride | |
| Ge | 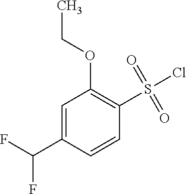 [SMILES: CCOc1cc(C(F)F)ccc1S(=O)(=O)Cl] | ¹H NMR (400 MHz, CHLOROFORM-d) δ 8.07 (d, J = 8.2 Hz, 1H), 7.36-7.30 (m, 1H), 6.96- 6.87 (m, 1H), 6.70 (t, J = 55.8 Hz, 1H), 4.36 (q, J = 7.0 Hz, 2H), 1.60 (t, J = 7.0 Hz, 3H) |
| | 4-(difluoromethyl)-2- | |
| | ethoxybenzene-1-sulfonyl chloride | |

### Intermediate H: 2-(Cyclopropyloxy)-6-methoxybenzene-1-sulfonyl chloride

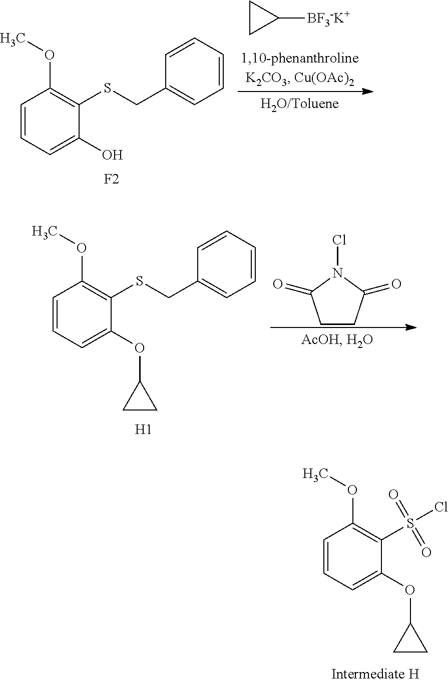

2-(Benzylsulfanyl)-1-(cyclopropyloxy)-3-methoxybenzene (H1): A mixture of 2-(benzylsulfanyl)-3-methoxyphenol (F2) (1.50 g, 6.09 mmol), potassium cyclopropyl-trifluoroborate (2.30 g, 15.5 mmol), 1,10-phenanthroline (274 mg, 1.52 mmol), copper (II) acetate (277 mg, 1.52 mmol), and potassium carbonate (1.68 g, 12.2 mmol) in toluene (12.0 mL) and H₂O (4.0 mL) was sparged with oxygen, then stirred at 70° C. under an oxygen balloon for 16 hrs. The mixture was diluted with EtOAc (50 mL) and the aqueous layer separated. The organic layer was concentrated and purified by silica gel chromatography (eluting with 0-10% EtOAc in Pet. ether) to give H1 (700 mg, 40%) as a yellow oil. ¹H NMR (400 MHZ, CHLOROFORM-d) δ 7.27-7.10 (m, 6H), 6.88 (dd, J=0.9, 8.3 Hz, 1H), 6.55 (d, J=8.6 Hz, 1H), 3.95 (s, 2H), 3.83 (s, 3H), 3.71 (br d, J=3.1 Hz, 1H), 0.81-0.72 (m, 2H), 0.72-0.63 (m, 2H). 2-(Cyclopropyloxy)-6-methoxybenzene-1-sulfonyl chloride (Intermediate H): By the same method used to convert G2 to Intermediate G, 2-(benzylsulfanyl)-1-(cyclopropyloxy)-3-methoxybenzene (H1) (900 mg, 3.14 mmol) was used to produce Intermediate H (700 mg, 85%) as a colorless oil which solidified on standing. ¹H NMR (400 MHZ, CHLOROFORM-d) δ 7.61-7.51 (m, 1H), 7.05 (dd, J=0.9, 8.4 Hz, 1H), 6.68 (d, J=8.6 Hz, 1H), 4.73 (s, 1H), 4.04-3.96 (m, 3H), 3.96-3.88 (m, 1H), 0.99-0.83 (m, 4H).

### Intermediate I: 3-(1,1-difluoroethyl)-1-[(4-methoxyphenyl)methyl]-1H-pyrazol-5-amine

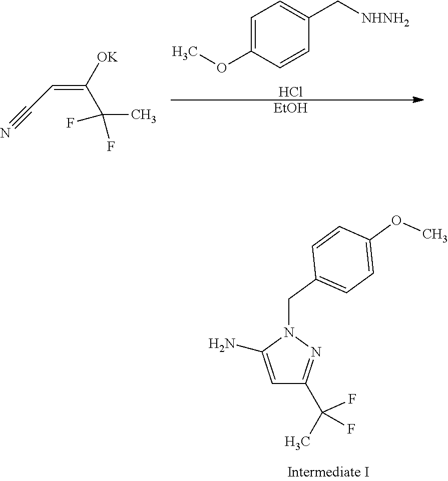

A mixture of potassium (E)-1-cyano-3,3-difluorobut-1-en-2-olate (171 mg, 1.0 mmol) and (4-methoxybenzyl) hydrazine hydrochloride (225 mg, 1.00 mmol) in ethanol (3.3 mL) was heated at 80° C. for six hrs. The crude reaction mixture was assayed by LCMS and gave a ˜3:2 mixture of isomers (Rₜ 1.2 min and Rₜ 1.5 min). The crude reaction mixture was concentrated to a solid and partitioned between EtOAc and H₂O. The EtOAc layer was washed with brine, dried with Na₂SO₄, filtered and concentrated to an oil. The crude product was purified by silica gel chromatography and eluted with 0-100% EtOAc-Heptane. Both isomers were separated on silica gel and the isomer with Rₜ 1.2 min was isolated as a light amber oil (189 mg, 71%). LCMS m/z 268 [M+H]⁺; ¹H NMR (400 MHZ, CHLOROFORM-d) δ 7.13 (d, J=8.6 Hz, 2H), 6.88 (d, J=8.6 Hz, 2H), 5.75 (s, 1H), 5.17 (s, 2H), 3.80 (s, 3H), 3.39 (br s, 2H), 2.00 (t, J=18.4 Hz, 3H).

### Intermediate J: 5-(Difluoromethoxy)-1-[(4-methoxyphenyl)methyl]-1H-pyrazol-3-amine

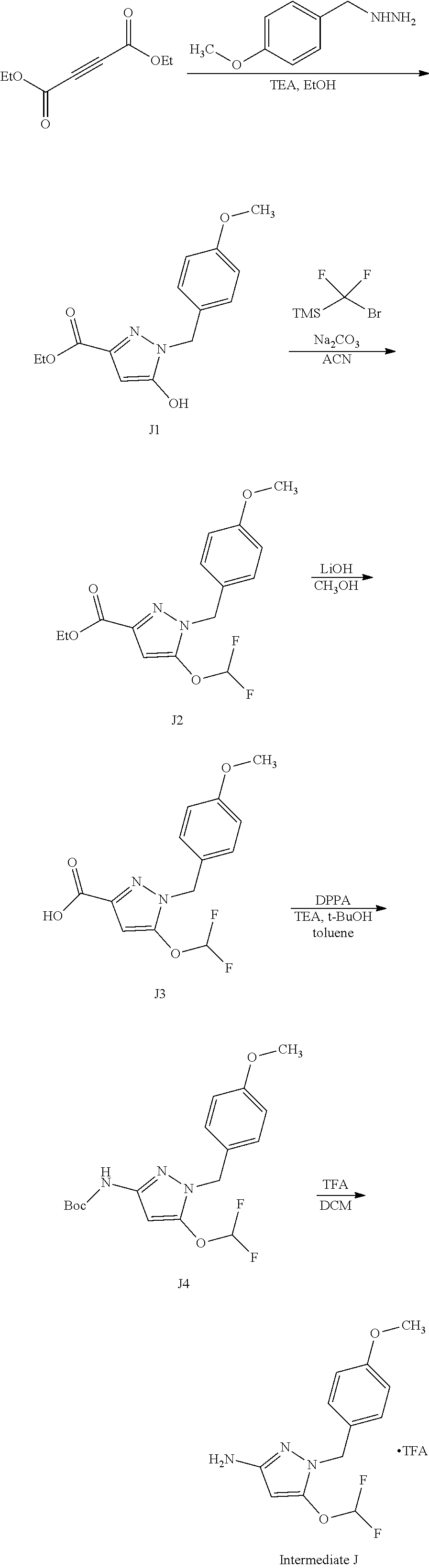

Ethyl 5-hydroxy-1-[(4-methoxyphenyl)methyl]-1H-pyrazole-3-carboxylate (J1): To a mixture of diethyl but-2-ynedioate (1.70 g, 10.0 mmol) and (4-methoxybenzyl) hydrazine (1.89 g, 10.0 mmol) in ethanol (20 mL) at rt was added TEA (1.1 g, 11.0 mmol, 1.5 mL). After two days, LCMS gave an 86:13 mixture of isomers (Rₜ 1.2 min (major) and Rₜ 1.4 min (minor)). The crude reaction mixture was concentrated and the crude product partitioned between EtOAc and H₂O. The EtOAc layer was washed with brine, dried with Na₂SO₄, filtered and concentrated to an oil. The crude product was purified by silica gel chromatography (40 g) and eluted with 0-100% EtOAc-heptane and gave J1 as a white solid (1.5 g, 54%). LCMS m/z 277 [M+H]⁺.

Ethyl 5-(difluoromethoxy)-1-[(4-methoxyphenyl)methyl]-1H-pyrazole-3-carboxylate (J2): A mixture of ethyl 5-hydroxy-1-[(4-methoxyphenyl)methyl]-1H-pyrazole-3-carboxylate (J1) (276 mg, 1.0 mmol) and sodium carbonate (116 mg, 1.1 mmol) in ACN (3.3 mL) was heated at 60° C. After 20 mins (bromodifluoromethyl) trimethylsilane (203 mg, 1.0 mmol, 155 μL) was added and a white precipitate formed. LCMS of the crude reaction mixture gave 36% conversion to product. More (bromodifluoromethyl) trimethylsilane (203 mg, 1.0 mmol, 155 μL) was added and after 45 mins, 57% conversion to product was observed. More (bromodifluoromethyl) trimethylsilane (203 mg, 1.0 mmol, 155 μL) was added. After 15 mins, the crude reaction mixture was cooled to rt and stirred overnight. The crude reaction mixture was assayed by LCMS and gave ˜11% starting material remaining, 61% product and 27% of an unknown side product. The crude reaction mixture was diluted with EtOAc and H₂O. The EtOAc layer was washed with brine, dried with Na₂SO₄, filtered and concentrated to an oil. The crude product was purified by silica gel chromatography (12 g) and eluted with 0-100% EtOAc-heptane to provide J2 as a white solid (176 mg, 54%). LCMS m/z 349 [M+Na]⁺; ¹H NMR (400 MHz, CHLOROFORM-d) δ 7.23 (d, J=8.8 Hz, 2H), 6.91-6.81 (m, 2H), 6.42 (s, 1H), 6.61-6.15 (m, 1H), 5.25 (s, 2H), 4.41 (q, J=7.1 Hz, 2H), 3.79 (s, 3H) 1.40 (t, J=7.1 Hz, 3H).

5-(Difluoromethoxy)-1-[(4-methoxyphenyl)methyl]-1H-pyrazole-3-carboxylic acid (J3): A mixture of ethyl 5-(difluoromethoxy)-1-[(4-methoxyphenyl)methyl]-1H-pyrazole-3-carboxylate (J2) (153 mg, 0.47 mmol) and lithium hydroxide hydrate (98.5 mg, 2.34 mmol) in methanol (1.56 mL) was stirred at rt for 20 mins then heated at 60° C. After one hour, LCMS gave no starting material and mostly product. The crude reaction mixture was concentrated to a white solid and partitioned between EtOAc and 1N aqueous HCl (3 mL). The EtOAc layer was washed with brine, dried with Na₂SO₄, filtered and concentrated to provide J3 as a white solid (141 mg, quantitative). LCMS m/z 321 [M+Na]⁺; ¹H NMR (400 MHZ, CHLOROFORM-d) δ 7.24 (d, J=8.7 Hz, 2H), 6.91-6.84 (m, 2H), 6.66-6.23 (m, 2H), 5.25 (s, 2H), 3.80 (s, 3H).

tert-Butyl {5-(difluoromethoxy)-1-[(4-methoxyphenyl)methyl]-1H-pyrazol-3-yl}carbamate (J4): To a suspension of 5-(difluoromethoxy)-1-[(4-methoxyphenyl)methyl]-1H-pyrazole-3-carboxylic acid (J3) (137 mg, 0.46 mmol) in toluene (1.53 mL) at rt was added TEA (51 mg, 0.5 mmol, 70 μL). A paste formed that stuck to the sides of the flask. Next, DPPA (139 mg, 0.50 mmol, 109 μL) was added and the mixture was heated at 90° C. for 16 hrs. LCMS gave a 2:1 mixture of the urea and the product. The crude reaction mixture was diluted with EtOAc and H₂O. The EtOAc layer was washed with brine, dried with Na₂SO₄, filtered and concentrated to an oil. The crude product was purified by silica gel chromatography (12 g) and eluted with 0-100% EtOAc-heptane to provide J4 as a colorless oil (36 mg, 22%). LCMS m/z 370 [M+H]⁺; ¹H NMR (400 MHZ, CHLOROFORM-d) δ 7.49 (br s, 1H), 7.15 (d, J=8.7 Hz, 2H), 6.84 (d, J=8.6 Hz, 2H), 6.66-6.26 (m, 1H), 6.22 (br s, 1H), 5.04 (s, 2H), 4.57-4.13 (m, 1H), 3.78 (s, 3H), 1.49 (s, 9H).

5-(Difluoromethoxy)-1-[(4-methoxyphenyl)methyl]-1H-pyrazol-3-amine (Intermediate J): A mixture of tert-butyl {5-(difluoromethoxy)-1-[(4-methoxyphenyl)methyl]-1H-pyrazol-3-yl}carbamate (J4) (36 mg, 0.10 mmol) and TFA (445 mg, 3.90 mmol, 0.3 mL) in DCM (0.3 mL) was stirred at rt for five hrs. The crude reaction mixture was concentrated to an oil (37 mg, quantitative) and H-NMR gave Intermediate J as the TFA salt. ¹H NMR (400 MHZ, CHLOROFORM-d) δ 7.23 (d, J=8.6 Hz, 2H), 6.93-6.81 (m, 2H), 6.73-6.35 (m, 1H), 6.26 (br s, 3H), 5.30 (s, 1H), 4.99 (s, 2H), 3.80 (s, 3H).

### Intermediate K: 5-(2,2-Difluorocyclopropyl)-1-[(4-methoxyphenyl)methyl]-1H-pyrazol-3-amine

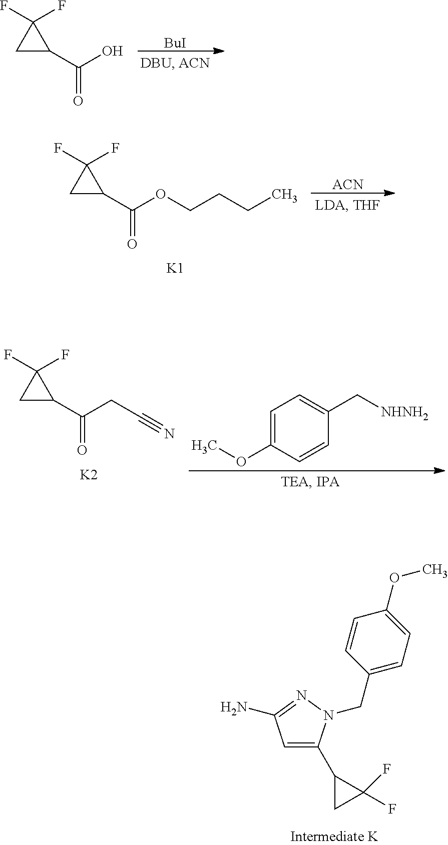

Butyl 2,2-difluorocyclopropane-1-carboxylate (K1): To a solution of 2,2-difluorocyclopropane-1-carboxylic acid (610 mg, 5.0 mmol) in ACN (10.0 mL) at rt was added 1,8-diazabicyclo [5.4.0]undec-7-ene (DBU, 837 mg, 5.5 mmol, 0.81 mL). The mixture was cooled in an ice water bath then butyliodide (1.0 g, 5.5 mmol, 0.63 mL) was added. After two days, the reaction mixture was concentrated to give a solid. The solid was partitioned between EtOAc and H₂O and the EtOAc layer was washed with brine, dried with Na₂SO₄, filtered and concentrated to provide K1 as an amber oil (499 mg, 56%). ¹H NMR (400 MHZ, CHLOROFORM-d) δ 4.16 (t, J=6.7 Hz, 2H), 2.45-2.41 (m, 1H), 2.11-2.03 (m, 1H), 1.84-1.70 (m, 1H), 1.67-1.57 (m, 2H), 1.40 (dq, J=15.0, 7.4 Hz, 2H), 0.95 (t, J=7.4 Hz, 3H).

3-(2,2-Difluorocyclopropyl)-3-oxopropanenitrile (K2): A solution of butyl 2,2-difluorocyclopropane-1-carboxylate (K1) (342 mg, 1.92 mmol) and ACN (158 mg, 3.84 mmol, 0.158 mL) in THF (0.91 mL) was cooled in a dry-ice acetone bath to −70° C. To the solution was added LDA (452 mg, 4.22 mmol, 2.11 mL, 2.0 M). A dark brown solution formed. After 30 mins, the crude reaction mixture was allowed to warm to rt. To the crude reaction mixture was added saturated aq NH₄Cl. The aqueous layer was partitioned between EtOAc and H₂O and the EtOAc layer was washed with brine, dried with Na₂SO₄, filtered and concentrated to an oil and the crude product K2 was used in the next step.

5-(2,2-Difluorocyclopropyl)-1-[(4-methoxyphenyl)methyl]-1H-pyrazol-3-amine (Intermediate K): The crude beta keto nitrile 3-(2,2-difluorocyclopropyl)-3-oxopropanenitrile (K2) was dissolved in isopropyl alcohol (2.0 mL), and (4-methoxybenzyl) hydrazine (362 mg, 1.9 mmol) was added, followed by TEA (194 mg, 1.92 mmol, 0.27 mL). The mixture was heated at 60° C. for 4 hrs and cooled to rt. The crude reaction mixture was concentrated to a solid. The solid was suspended in DCM and loaded onto a 5 g silica gel pre-column cartridge. LCMS of the solid confirmed it to be the TEA-HCl salt and it was discarded. The crude product was purified by silica gel chromatography (pre-column in series with a 12 g silica gel column) and eluted with 0-100% EtOAc-heptane to provide Intermediate K as a yellow oil (76 mg, 14%). LCMS m/z 280 [M+H]⁺; ¹H NMR (400 MHZ, CHLOROFORM-d) δ 7.11 (d, J=8.6 Hz, 2H), 6.90-6.84 (m, 2H), 5.42 (d, J=1.1 Hz, 1H), 5.11 (s, 2H), 3.79 (s, 3H), 3.55-3.08 (m, 2H), 2.69 (td, J=12.2, 8.0 Hz, 1H), 1.83-1.74 (m, 1H), 1.70-1.62 (m, 1H).

The amino pyrazole intermediates in Table 17 were prepared according to the general method of Intermediate K, using commercially available acids or esters and either excess hydrazine in an alcoholic solvent or (4-methoxybenzyl) hydrazine.

TABLE 17

| Intermediate | | |
|---|---|---|
| Designation | Structure/IUPAC name | Analytical Data |
| Ka | 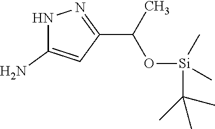 [SMILES: CC(O[Si](C)(C)C(C)(C)C)c1cc(N)[nH]n1] | LCMS m/z 242 [M + H]⁺; ¹H NMR (400 MHz, CHLOROFORM-d) δ 7.27 (s, 1H), 5.43 (s, 1H), 4.92 (q, J = 6.5 Hz, 1H), 1.43 (d, J = 6.5 Hz, 3H), 0.92 (m, 9H), 0.07 (s, 3H), 0.01 (s, 3H) |
| | 3-(1-{[tert-butyl(dimethyl)silyl]oxy}ethyl)- | |
| | 1 H-pyrazol-5-amine | |
| Kb | 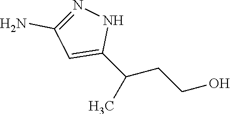 | LCMS m/z 156 [M + H]⁺; ¹H NMR (400 MHz, DMSO-d₆) δ 8.94-8.87 (m, 1H), 5.17 (s, 1H), 4.36 (br s, 3H), 3.38-3.26 (m, 2H), 2.79-2.71 (m, 1H), 1.71-1.64 (m, 1H), 1.60-1.52 (m, 1H), 1.12 (d, J = 7.1 Hz, 3H) |
| | 3-(3-amino-1 H-pyrazol-5-yl)butan-1-ol | |

### Intermediate L: 4-Methyl-5,6-dihydro-4H-pyrrolo[1,2-b]pyrazol-2-amine

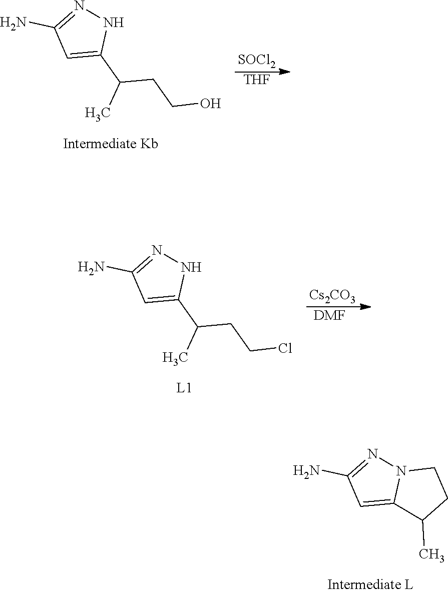

5-(4-Chlorobutan-2-yl)-1H-pyrazol-3-amine (L1): To a round bottom flask was added 3-(3-amino-1H-pyrazol-5-yl) butan-1-ol (Intermediate Kb) (183 mg, 1.18 mmol) and THF (7.8 mL) followed by thionyl chloride (701 mg, 5.9 mmol, 0.43 mL). The resultant milky white mixture was stirred for two hrs at rt. LCMS gave a ˜1:1 mixture of L1 and Intermediate L, LCMS m/z 174 [M+H]⁺ and 138 [M+H]⁺, respectively.

4-Methyl-5,6-dihydro-4H-pyrrolo[1,2-b]pyrazol-2-amine (Intermediate L): The crude reaction mixture from the preparation of L1 was concentrated followed by the addition of DMF (6.6 mL) and cesium carbonate (576 mg, 1.8 mmol). The mixture was placed in a pre-heated hot plate at 100° C. and stirred at 100° C. for one hour. LCMS gave full conversion to product. The crude reaction mixture was cooled to rt and filtered. The filtrate was concentrated and triturated with EtOAc and methanol (˜5:1) which gave Intermediate L as a beige solid (68 mg, 42%). LCMS m/z 138 [M+H]⁺.

### Intermediate M: 5′,6′-Dihydrospiro[cyclopropane-1,4′-pyrrolo[1,2-b]pyrazol]-2′-amine

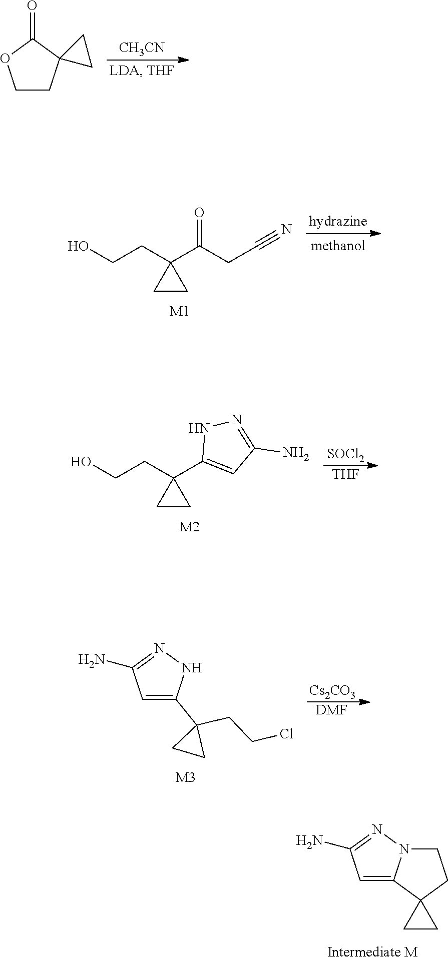

3-[1-(2-Hydroxyethyl)cyclopropyl]-3-oxopropanenitrile (M1): To a sealed oven dried vial flushed with nitrogen was added LDA (0.49 mL, 0.98 mmol) and anhydrous THF (1 mL). The solution was degassed, backfilled three times with nitrogen and cooled to −78° C. A degassed and nitrogen flushed solution of 5-oxaspiro[2.4]heptan-4-one (42 μL, 0.45 mmol) in anhydrous THF (1.4 mL) and anhydrous ACN (47 μL, 0.89 mmol) was added dropwise to the solution of LDA in THF. The solution was stirred at −78° C. for 15 mins, then the vial was warmed to rt and stirred for two hrs which gave a pale yellow mixture. The crude reaction mixture was diluted with aq saturated ammonium chloride solution and the aqueous layer was extracted three times with EtOAc. The combined organic extracts were washed with brine, dried over sodium sulfate, filtered and concentrated which gave M1 as an orange oil. The crude product was used in the next step.

2-[1-(3-Amino-1H-pyrazol-5-yl)cyclopropyl]ethan-1-ol (M2): To a vial was added 3-[1-(2-hydroxyethyl)cyclopropyl]-3-oxopropanenitrile (M1) (68 mg, 0.44 mmol) and methanol (0.63 mL). To the solution was added hydrazine monohydrate (32 μL, 0.67 mmol) and the yellow solution was stirred overnight at 80° C. LCMS gave product. The crude reaction mixture was cooled to rt and concentrated to an orange oil. The crude product was purified using silica gel chromatography (4 g) and eluted with 0-20% methanol-DCM to provide M2 as a yellow oil (26 mg, 35%). ¹H NMR (METHANOL-d₄, 400 MHZ) δ 5.31 (s, 1H), 3.50 (t, J=7.3 Hz, 2H), 1.70 (t, J=7.3 Hz, 2H), 0.80-0.70 (m, 2H), 0.69-0.60 (m, 2H).

5-[1-(2-Chloroethyl)cyclopropyl]-1H-pyrazol-3-amine (M3): To a vial was added 2-[1-(3-amino-1H-pyrazol-5-yl)cyclopropyl]ethan-1-ol (M2) (26 mg, 0.16 mmol) in anhydrous THF (86 mL) and thionyl chloride (58 μL, 0.78 mmol). The crude reaction mixture was stirred at rt for two hrs. The yellow solution quickly became a cloudy mixture, then an intense yellow solution. LCMS gave 2 to 1, 5-[1-(2-chloroethyl)cyclopropyl]-1H-pyrazol-3-amine (M3) to 5′,6′-dihydrospiro[cyclopropane-1,4′-pyrrolo[1,2-b]pyrazol]-2′-amine (Intermediate M). After 18 hrs, the LCMS trace gave the same as after two hrs. The crude reaction mixture was concentrated and provided a dark orange oil and used in the next step.

### Intermediate M: 5′,6′-Dihydrospiro[cyclopropane-1,4′-pyrrolo[1,2-b]pyrazol]-2′-amine

The crude product was dissolved in DMF (0.86 mL) and Cs₂CO₃ (76 mg, 0.23 mmol) was added. The crude reaction mixture was heated at 100° C. for one hour. LCMS of the resultant orange mixture gave full conversion of 5-[1-(2-chloroethyl)cyclopropyl]-1H-pyrazol-3-amine (M3) to 5′,6′-dihydrospiro[cyclopropane-1,4′-pyrrolo[1,2-b]pyrazol]-2′-amine (Intermediate M). The crude reaction mixture was cooled to rt and added dropwise to H₂O (75 mL). The aqueous layer was extracted three times with DCM. An emulsion formed which was separated by adding a scoop of LiCl to break the emulsion. A LCMS of the aqueous layer showed that the product remained in the aqueous layer, even after extracting three times with EtOAc. Thus, the organic layers and aqueous layers were combined and lyophilized over the weekend which provided an orange solid. The crude product was purified by reverse phase HPLC (5-45% ACN-H₂O gradient over 25 mins) which gave Intermediate M (6 mg, 25%). ¹H NMR (METHANOL-d₄, 400 MHz) δ 4.94 (s, 1H), 3.91 (t, J=7.2 Hz, 2H), 2.38 (dt, J=7.3, 1.0 Hz, 2H), 0.86 (s, 4H).

### Intermediate N: 5-Cyclopropyl-1-(oxan-2-yl)-1H-pyrazol-3-amine

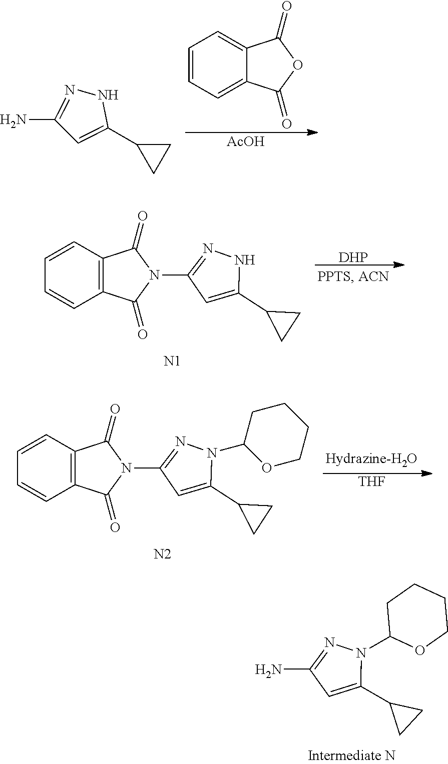

2-(5-cyclopropyl-1H-pyrazol-3-yl)-1H-isoindole-1,3 (2H)-dione (N1): A mixture of 5-cyclopropyl-1H-pyrazol-3-amine (10 g, 81 mmol) and 2-benzofuran-1,3-dione (12 g, 81 mmol) in acetic acid was heated at 100° C. for two hrs and 120° C. for six hrs. The crude reaction mixture was concentrated under reduced pressure and H₂O was added. A white solid was collected by filtration (19 g, 92%). LCMS m/z 254 [M+H]⁺.

2-[5-cyclopropyl-1-(oxan-2-yl)-1H-pyrazol-3-yl]-1H-isoindole-1,3 (2H)-dione (N2): To a suspension of 2-(5-cyclopropyl-1H-pyrazol-3-yl)-1H-isoindole-1,3 (2H)-dione (N1) (17.9 g, 71 mmol) in ACN (353 mL) was added pyridinium para-toluenesulfonate (2.7 g, 11 mmol) and dihydro-2H-pyran (12 g, 141 mmol). The reaction was stirred at 60° C. for 16 hrs. The crude reaction mixture was combined with a smaller batch (1 g of N1 was used) and concentrated under reduced pressure. The crude product was triturated with H₂O (300 mL) and EtOAc (50 mL), and filtered. The solids were washed with H₂O and EtOAc (50 mL×3), and dried to afford N2 as a white solid (19.8 g, 83.0%). The filtrate was extracted with EtOAc (100 mL×2), washed with brine (200 mL), concentrated at 66° C. under house vacuum and then high vacuum to give the crude product as a light red oil, which was triturated with Pet. ether:EtOAc=3:1 (100 mL) for 2 hrs, filtered and the collected solid was washed with H₂O and Pet. ether:EtOAc=3:1 (10 mL×3), and dried to afford additional N2 as an off-white solid (2.9 g, 12%). LCMS m/z 260 [M+Na]⁺; ¹H NMR (400 MHZ, DMSO-d₆) δ 7.99-7.89 (m, 4H), 6.06 (s, 1H), 5.64 (dd, J=9.9, 2.3 Hz, 1H), 3.98-2.94 (m, 1H), 3.75-3.63 (m, 1H), 2.33-2.19 (m, 1H), 2.09-1.96 (m, 2H), 1.96-1.87 (m, 1H), 1.77-1.61 (m, 1H), 1.61-1.48 (m, 2H), 1.07-0.96 (m, 2H), 0.79-0.64 (m, 2H). 5-Cyclopropyl-1-(oxan-2-yl)-1H-pyrazol-3-amine (Intermediate N): A solution of 2-[5-cyclopropyl-1-(oxan-2-yl)-1H-pyrazol-3-yl]-1H-isoindole-1,3 (2H)-dione (N2) (22.7 g, 67 mmol) in THF (336 mL) was treated with hydrazine monohydrate (9.5 g, 150 mmol) and stirred at 70° C. for 4 hrs. After cooling to ambient temperature, the suspension was filtered. The filtrate was extracted with EtOAc (200 mL×6) and the combined organic phases were washed with saturated sodium bicarbonate (200 mL×2) and brine (200 mL×2), dried over sodium sulfate and concentrated under reduced pressure to afford Intermediate N as a yellow oil (9.8 g, 70%). The combined aqueous phases were extracted with EtOAc (200 mL×6) again. The combined organic phases were washed with saturated sodium bicarbonate (100 mL) and brine (200 mL×2), dried over sodium sulfate and concentrated under reduced pressure to afford more Intermediate N as a yellow oil (2.84 g, 20%). LCMS m/z 208 [M+H]⁺; ¹H NMR (400 MHZ, CDCl₃) δ 5.35 (dd, J=2.4, 10.5 Hz, 1H), 5.28 (s, 1H), 4.15-4.04 (m, 1H), 3.92-3.20 (m, 3H), 2.47-2.30 (m, 1H), 2.17-2.00 (m, 1H), 1.91-1.82 (m, 1H), 1.81-1.64 (m, 3H), 1.58-1.51 (m, 1H), 0.93-0.90 (m, 2H), 0.78-0.69 (m, 1H), 0.65-0.55 (m, 1H).

### EXAMPLES

### Method A

### Example 01:2,6-Dimethoxy-N-{4-methoxy-6-[(1H-pyrazol-3-yl)amino]-1,2-benzoxazol-3-yl}benzene-1-sulfonamide

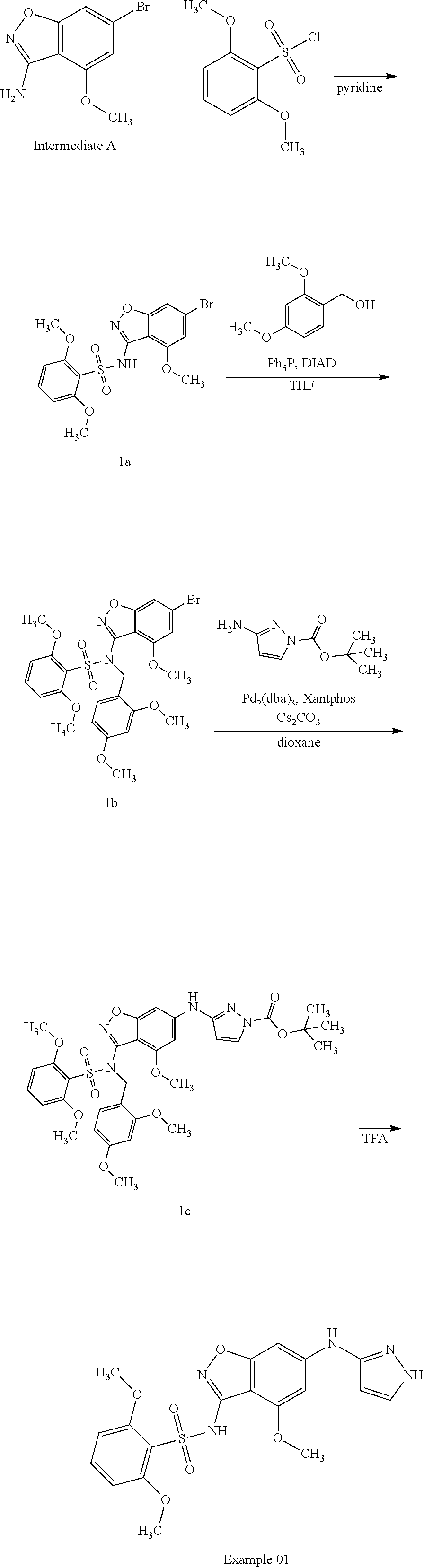

N-(6-bromo-4-methoxy-1,2-benzoxazol-3-yl)-2,6-dimethoxybenzene-1-sulfonamide (1a): A solution of 6-bromo-4-methoxy-1,2-benzoxazol-3-amine (Intermediate A) (17 g, 70 mmol) and 2,6-dimethoxybenzene-1-sulfonyl chloride (24.8 g, 105 mmol) in pyridine (170 mL) was heated at 120° C. for two hrs. After cooling to rt, the solvent was evaporated under reduced pressure and the residue purified by silica gel chromatography (eluting with 100% EtOAc) to give 1a (26.5 g, 85%) as a solid. LCMS m/z 443/445 [M+H]⁺; ¹H NMR (400 MHZ, DMSO-d₆) δ 9.78 (s, 1H), 7.56-7.45 (m, 2H), 7.05 (d, J=1.1 Hz, 1H), 6.78 (d, J=8.4 Hz, 2H), 3.93 (s, 3H), 3.77 (s, 6H).

N-(6-bromo-4-methoxy-1,2-benzoxazol-3-yl)-N-[(2,4-dimethoxyphenyl)methyl]-2,6-dimethoxybenzene-1-sulfonamide (1b): A solution of N-(6-bromo-4-methoxy-1,2-benzoxazol-3-yl)-2,6-dimethoxybenzene-1-sulfonamide (1a) (28.0 g, 63 mmol), (2,4-dimethoxyphenyl)-methanol (15.9 g, 94.8 mmol), and triphenylphosphine (41.4 g, 158 mmol) in THF (300 mL) was cooled to 0° C., then DIAD (25.5 g, 126 mmol) was added dropwise. After stirring at rt (15° C.) for 16 hrs, the pale-yellow solution was concentrated under reduced pressure and purified by silica gel chromatography (eluting with 60-70% EtOAc in Pet. ether) to give ˜25 g of product which was contaminated with triphenylphosphine oxide. Recrystallization from methanol afforded 1b (7.0 g, 19%) as a white solid. The mother liquor was concentrated and purified by reverse-phase preparative HPLC (YMC-Triart Prep C18 250×50 mm, 10 μm column, eluting with 50-70% H₂O+0.1% TFA in ACN). The product-containing fractions were concentrated to remove ACN, extracted with EtOAc, washed with brine, dried over sodium sulfate, and concentrated to give a second batch of 1b (8.30 g, 22%) as a solid. ¹H NMR (400 MHZ, DMSO-d₆) δ 7.64 (d, J=1.1 Hz, 1H), 7.56 (t, J=8.4 Hz, 1H), 7.22 (d, J=8.4 Hz, 1H), 7.03 (d, J=1.0 Hz, 1H), 6.77 (d, J=8.6 Hz, 2H), 6.45 (d, J=8.0 Hz, 1H), 6.39 (s, 1H), 4.78 (s, 2H), 3.70 (s, 3H), 3.65 (s, 3H), 3.52 (s, 6H), 3.39 (s, 3H).

tert-Butyl 3-[(3-{(2,6-dimethoxybenzene-1-sulfonyl)[(2,4-dimethoxyphenyl)methyl]amino}-4-methoxy-1,2-benzoxazol-6-yl)amino]-1H-pyrazole-1-carboxylate (1c): To a solution of N-(6-bromo-4-methoxy-1,2-benzoxazol-3-yl)-N-[(2,4-dimethoxyphenyl)methyl]-2,6-dimethoxybenzene-1-sulfonamide (1b) (100.0 mg, 0.169 mmol) and tert-butyl 3-amino-1H-pyrazole-1-carboxylate (92.6 mg, 0.506 mmol) in 1,4-dioxane (2 mL) was added cesium carbonate (165 mg, 0.506 mmol), 4,5-bis(diphenylphosphino)-9,9-dimethylxanthene (Xantphos, 29.3 mg, 0.051 mmol) and tris(dibenzylideneacetone)dipalladium(0) (23.1 mg, 0.025 mmol). The suspension was stirred at 100° C. for 16 hrs, then concentrated under vacuum to give crude 1c (150 mg, >100%) as a brown gum, which was used in the next step without further purification. LCMS m/z 696 [M+H]⁺.

2,6-Dimethoxy-N-{4-methoxy-6-[(1H-pyrazol-3-yl)amino]-1,2-benzoxazol-3-yl}benzene-1-sulfonamide (Example 01): Crude tert-butyl 3-[(3-{(2,6-dimethoxybenzene-1-sulfonyl)[(2,4-dimethoxyphenyl)methyl]amino}-4-methoxy-1,2-benzoxazol-6-yl)amino]-1H-pyrazole-1-carboxylate (1c) (100 mg, maximum 0.113 mmol) was dissolved in DCM (2 mL) and TFA (2 mL), and stirred at rt (20° C.) for 15 hrs. The resulting pink solution was concentrated to dryness, the residue was dissolved in methanol (2 mL) and DMSO (2 mL), the solution was filtered and purified by preparative reverse-phase HPLC (YMC Triart C18, 250×50 mm×7 um column, eluting with 11-51% H₂O+0.2% formic acid in ACN). The product-containing fractions were lyophilized to give Example 01 (40 mg, 80%) as a white solid. LCMS m/z 446.1 [M+H]⁺; ¹H NMR (400 MHZ, DMSO-d₆) δ 9.18 (s, 1H), 9.10 (s, 1H), 7.62 (d, J=2.2 Hz, 1H), 7.50 (t, J=8.5 Hz, 1H), 7.25 (s, 1H), 6.78 (d, J=8.4 Hz, 2H), 6.63 (s, 1H), 5.90 (d, J=2.1 Hz, 1H), 3.88 (s, 3H), 3.79 (s, 6H).

### Method B

### Example 02: N-{6-[(3-ethyl-1H-pyrazol-5-yl)amino]-5-methoxy-1,2-benzoxazol-3-yl}-2,6-dimethoxybenzene-1-sulfonamide

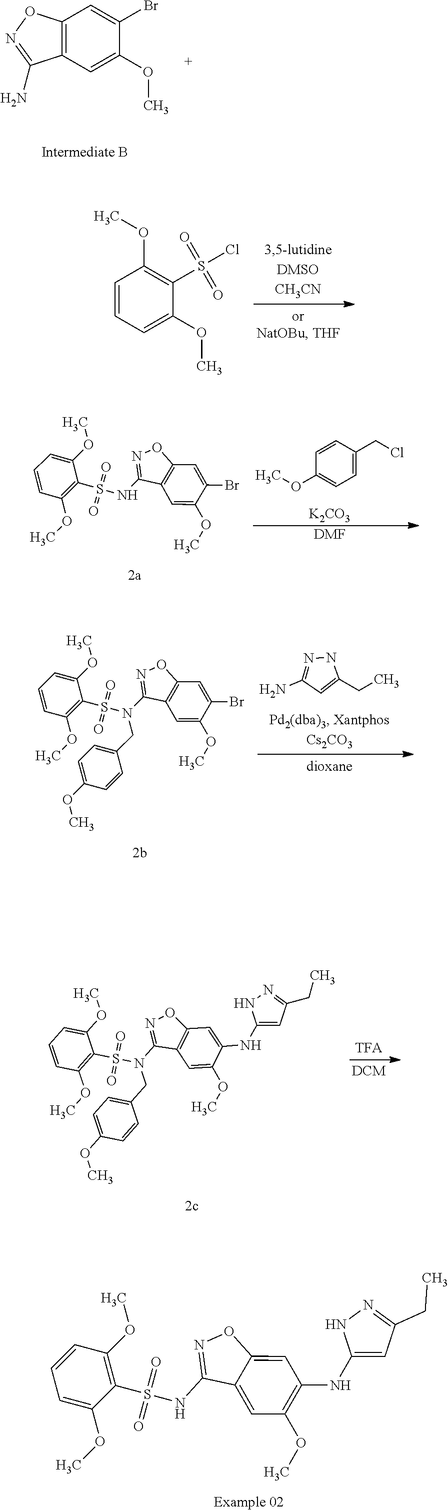

N-(6-bromo-5-methoxy-1,2-benzoxazol-3-yl)-2,6-dimethoxybenzene-1-sulfonamide (2a): A sealable reaction vessel was charged with 6-bromo-5-methoxy-1,2-benzoxazol-3-amine

(Intermediate B) (1.50 g, 6.17 mmol) and 2,6-dimethoxybenzene-1-sulfonyl chloride (1.75 g, 7.41 mmol). A 0.05 M solution of DMSO in ACN (6.17 mL, 0.309 mmol DMSO) was added, followed by a solution of 3,5-lutidine (1.98 g, 2.11 mmol) in ACN (15.0 mL). The vessel was sealed and stirred at 23° C. for 20 hrs. A solid precipitate formed during this time. Since unreacted Intermediate B was still present by LCMS, a second portion of 2,6-dimethoxybenzene-1-sulfonyl chloride (906 mg, 3.83 mmol) and 3,5-lutidine (992 mg, 9.26 mmol) were added, the vessel resealed, and stirring continued at 23° C. for an additional 20 hrs. The reaction mixture was then diluted with DCM (100 mL) and washed with 2N aqueous HCl (35 mL). The organic layer was washed with saturated aq NaHCO₃ (50 mL), and a white solid precipitate formed. The suspension was filtered, and the filter cake washed with H₂O (3×10 mL) and DCM (3×5 mL). The precipitate was dried under vacuum overnight, then suspended in ACN (5 mL) and H₂O (30 mL) and lyophilized to dryness, leaving 2a (2.1 g, 77%) as a white solid. LCMS m/z 443/445 [M+H]⁺; ¹H NMR (400 MHZ, DMSO-d₆) δ 7.65 (s, 1H), 7.28 (s, 1H), 7.24 (t, J=8.3 Hz, 1H), 6.61 (d, J=8.4 Hz, 2H), 3.83 (s, 3H), 3.59 (s, 6H).

Alternatively, a reaction vessel was charged with 6-bromo-5-methoxy-1,2-benzoxazol-3-amine (Intermediate B) (0.70 g, 2.88 mmol) and 2,6-dimethoxybenzene-1-sulfonyl chloride (1.02 g, 4.32 mmol) in ACN (15.0 mL). The vessel was stirred at 23° C. and a 2.0 M solution of sodium tert-butoxide in THF (5.0 mL, 10.1 mmol) was added dropwise. A solid precipitate formed during this time. The reaction mixture was stirred for two hrs then diluted with EtOAc (50 mL) and washed with 1N aqueous HCl (35 mL). The organic layer was washed with brine (30 mL) dried over sodium sulfate and concentrated. The solid residue was slurried in heptane, filtered under vacuum and washed with heptane leaving 2a (1.3 g, 99%) as a white solid. LCMS m/z 443/445 [M+H]⁺; ¹H NMR (400 MHZ, DMSO-d₆) δ 11.35 (s, 1H), 8.00 (s, 1H), 7.73 (s, 1H), 7.48 (t, J=8.4 Hz, 1H), 6.74 (d, J=8.5 Hz, 2H), 3.87 (s, 3H), 3.74 (s, 6H).

N-(6-bromo-5-methoxy-1,2-benzoxazol-3-yl)-2,6-dimethoxy-N-[(4-methoxyphenyl)methyl]benzene-1-sulfonamide (2b): A mixture of N-(6-bromo-5-methoxy-1,2-benzoxazol-3-yl)-2,6-dimethoxybenzene-1-sulfonamide (2a) (2.78 g, 6.17 mmol), 4-methoxybenzyl chloride (1.06 g, 6.79 mmol), and potassium carbonate (1.02 g, 7.40 mmol) in DMF (30.0 mL) was stirred at 50° C. for 16 hrs. The mixture was poured into H₂O (100 mL) and stirred for 20 mins. The resulting suspension was filtered, and the filter cake washed with H₂O (5 mL). The solids were taken up in ACN (50 mL) and evaporated to dryness (three cycles), yielding 2b (3.0 g, 86%) as a white solid. LCMS m/z 563/565 [M+H]⁺; ¹H NMR (400 MHZ, DMSO-d₆) δ 8.09 (s, 1H), 7.57 (t, J=8.4 Hz, 1H), 7.30 (d, J=8.7 Hz, 2H), 7.24 (s, 1H), 6.88-6.83 (m, 2H), 6.81 (d, J=8.6 Hz, 2H), 4.98 (s, 2H), 3.78 (s, 3H), 3.72 (s, 6H), 3.69 (s, 3H).

N-{6-[(3-ethyl-1H-pyrazol-5-yl)amino]-5-methoxy-1,2-benzoxazol-3-yl}-2,6-dimethoxy-N-[(4-methoxyphenyl)methyl]benzene-1-sulfonamide (2c): A yellow suspension of N-(6-bromo-5-methoxy-1,2-benzoxazol-3-yl)-2,6-dimethoxy-N-[(4-methoxyphenyl)methyl]benzene-1-sulfonamide (2b) (150 mg, 0.266 mmol), 5-ethyl-1H-pyrazol-3-amine (188 mg, 1.69 mmol), cesium carbonate (260 mg, 0.799 mmol), 4,5-bis(diphenylphosphino)-9,9-dimethylxanthene (Xantphos, 108 mg, 0.186 mmol) and tris(dibenzylideneacetone)dipalladium(0) (48.8 mg, 0.0532 mmol) in 1,4-dioxane (6.0 mL) was sparged with dry argon for one minute, then the mixture heated at 100° C. for 16 hrs. After cooling to rt, the reaction mixture was diluted with H₂O (30 mL) and stirred for 30 mins. The resulting solids were collected by filtration. The filter cake was washed with H₂O (2×5 mL), then suspended in DCM (100 mL) with stirring for 10 mins. The suspension was filtered, and the solids washed with methanol (4×1 mL). The filtrate was concentrated under vacuum and purified by silica gel chromatography (eluting with 0-100% EtOAc in Pet. ether) to give 2c (130 mg, 82%) as a yellow glass. LCMS m/z 594 [M+H]⁺; 1H NMR (400 MHZ, DMSO-d₆) δ 11.89 (s, 1H), 8.16 (d, J=12.5 Hz, 2H), 7.54 (t, J=8.4 Hz, 1H), 7.32 (d, J=8.5 Hz, 2H), 6.97 (s, 1H), 6.87 (d, J=8.8 Hz, 2H), 6.79 (d, J=8.5 Hz, 2H), 5.88 (d, J=1.8 Hz, 1H), 4.99 (s, 2H), 3.77 (s, 3H), 3.75 (s, 6H), 3.70 (s, 3H), 2.56 (q, J=7.7 Hz, 2H), 1.18 (t, J=7.5 Hz 3H).

N-{6-[(3-ethyl-1H-pyrazol-5-yl)amino]-5-methoxy-1,2-benzoxazol-3-yl}-2,6-dimethoxybenzene-1-sulfonamide (Example 02): TFA (1 mL) was added to a cooled (0° C.) solution of N-{6-[(3-ethyl-1H-pyrazol-5-yl)amino]-5-methoxy-1,2-benzoxazol-3-yl}-2,6-dimethoxy-N-[(4-methoxyphenyl)methyl]benzene-1-sulfonamide (2c) (130 mg, 0.219 mmol) in DCM (4 mL). After stirring at rt (25° C.) for five hrs, the solvent was evaporated, and the residue suspended and stirred in methanol (2 mL) and ACN (2 mL) for five mins. The solids were collected by filtration and washed with ACN (3×1 mL). The filter cake was dissolved in H₂O (20 mL) and DCM (20 mL), stirred for 10 mins, and adjusted to pH 10 with saturated aq NaHCO₃. The layers were separated, and the aqueous layer extracted with DCM/methanol (10:1, 4×25 mL). The combined organic layers were dried over sodium sulfate, filtered, concentrated, and purified by reverse-phase preparative HPLC (YMC Triart C1; 8 250×50 mm, 7 μm column, eluting with 0-40% H₂O+0.05% NH₄OH in ACN) to give Example 02 (55 mg, 53%) as a white solid. LCMS m/z 474.1 [M+H]⁺; ¹H NMR (400 MHZ, DMSO-d₆) δ 11.89 (s, 1H), 10.97 (br s, 1H), 8.16 (s, 1H), 8.10 (s, 1H), 7.46 (t, J=8.4 Hz, 1H), 7.40 (s, 1H), 6.74 (d, J=8.6 Hz, 2H), 5.89 (s, 1H), 3.88 (s, 3H), 3.76 (s, 6H), 2.57 (q, J=7.7 Hz, 2H), 1.18 (t, J=7.6 Hz, 3H).

### Method C

### Example 03:2,6-Dimethoxy-N-{5-methoxy-6-[(1H-pyrazol-3-yl)amino]-1,2-benzoxazol-3-yl}benzene-1-sulfonamide

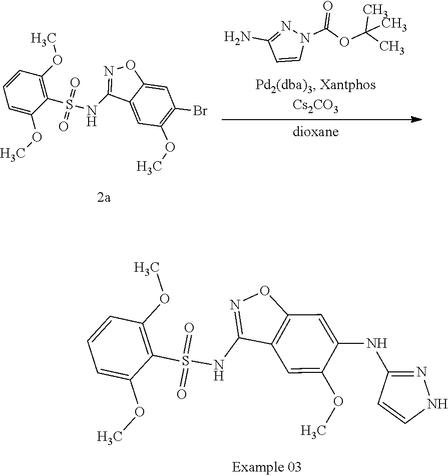 [SMILES: COc1cc2c(NS(=O)(=O)c3c(OC)cccc3OC)noc2cc1Br ; CC(C)(C)OC(=O)n1ccc(N)n1 ; COc1cc2c(NS(=O)(=O)c3c(OC)cccc3OC)noc2cc1Nc1cc[nH]n1]

A solution of N-(6-bromo-5-methoxy-1,2-benzoxazol-3-yl)-2,6-dimethoxybenzene-1-sulfonamide (2a, as prepared in Method B) (50.0 mg, 0.11 mmol), tert-butyl 3-amino-1H-pyrazole-1-carboxylate (62.0 mg, 0.338 mmol), cesium carbonate (110 mg, 0.338 mmol), 4,5-bis(diphenylphosphino)-9,9-dimethylxanthene (Xantphos, 19.6 mg, 0.0338 mmol) and tris(dibenzylideneacetone)dipalladium(0) (15.5 mg, 0.0169 mmol) in 1,4-dioxane (2.0 mL) was stirred at 100° C. for 16 hrs. A yellow precipitate formed. The suspension was diluted with EtOAc (10 mL), filtered to remove solids, and the filtrate concentrated to dryness under vacuum. The residue was purified by reverse-phase preparative HPLC (YMC Triart C18; 250×50 mm, 7 um column, eluting with 17-57% H₂O+0.05% NH₄OH in ACN), affording Example 03 (20 mg, 40%) as a pale-yellow solid. LCMS m/z 446.1 [M+H]⁺, 468 [M+Na]⁺; ¹H NMR (400 MHZ, DMSO-d₆) δ 12.17 (br s, 1H), 8.21 (s, 1H), 8.15 (br d, J=4.6 Hz, 1H), 7.59 (d, J=2.3 Hz, 1H), 7.46 (t, J=8.4 Hz, 1H), 7.41 (s, 1H), 6.74 (d, J=8.5 Hz, 2H), 6.11 (d, J=2.3 Hz, 1H), 3.89 (s, 3H), 3.76 (s, 6H).

### Example 04:2,6-Dimethoxy-N-{5-methoxy-6-[(1-methyl-1H-pyrazol-3-yl)amino]-1,2-benzoxazol-3-yl}benzene-1-sulfonamide

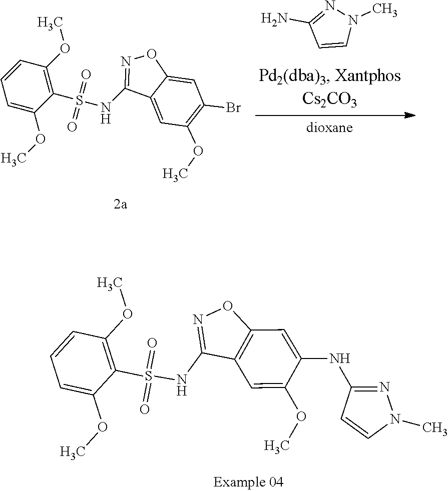

A solution of N-(6-bromo-5-methoxy-1,2-benzoxazol-3-yl)-2,6-dimethoxybenzene-1-sulfonamide (2a, as prepared in Method B) (50.0 mg, 0.11 mmol), 1-methyl-1H-pyrazol-3-amine (32.9 mg, 0.338 mmol), cesium carbonate (110 mg, 0.338 mmol), 4,5-bis(diphenylphosphino)-9,9-dimethylxanthene (Xantphos, 19.6 mg, 0.0338 mmol) and tris(dibenzylideneacetone) dipalladium(0) (15.5 mg, 0.0169 mmol) in 1,4-dioxane (2.0 mL) was stirred at 100° C. for 16 hrs. After cooling to rt, the mixture was diluted with EtOAc (10 mL), the solids were filtered off, the filtrate concentrated to dryness, and the residue purified by reverse-phase preparative HPLC (YMC Triart C18; 250×50 mm, 7 um column, eluting with 24-64% H₂O+0.2% formic acid in ACN), yielding Example 04 (30 mg, 52%) as a pale yellow solid. LCMS m/z 460.1 [M+H]⁺; 1H NMR (400 MHZ, DMSO-d₆) δ 10.92 (br s, 1H), 8.23 (s, 1H), 8.15 (s, 1H), 7.53 (d, J=2.3 Hz, 1H), 7.47 (t, J=8.4 Hz, 1H), 7.41 (s, 1H), 6.74 (d, J=8.5 Hz, 2H), 6.06 (d, J=2.4 Hz, 1H), 3.88 (s, 3H), 3.79 (s, 3H), 3.76 (s, 6H).

### Example 05:3-Methoxy-N-{5-methoxy-6-[(1H-pyrazol-3-yl)amino]-1,2-benzoxazol-3-yl}-5,6,7,8-tetrahydronaphthalene-2-sulfonamide

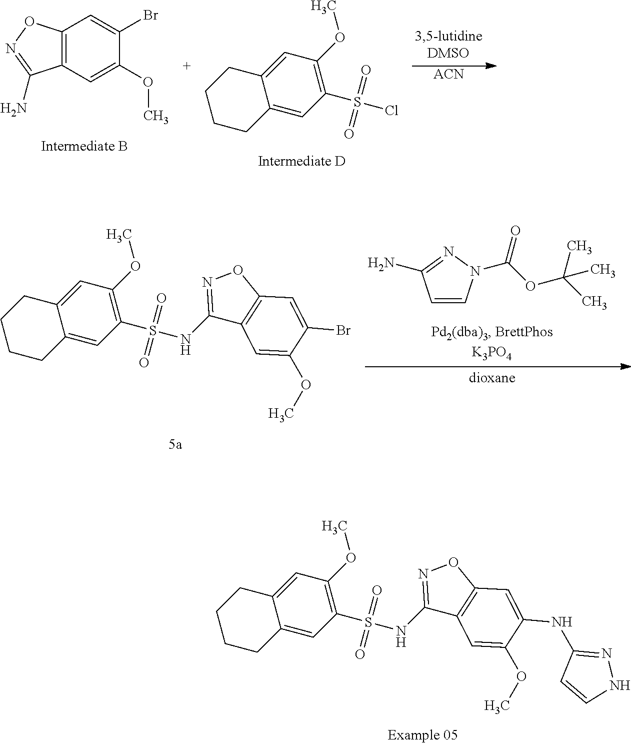

N-(6-Bromo-5-methoxy-1,2-benzoxazol-3-yl)-3-methoxy-5,6,7,8-tetrahydronaphthalene-2-sulfonamide (5a): By the same method used to synthesize 2a, 6-bromo-5-methoxy-1,2-benzoxazol-3-amine (Intermediate B) (257 mg, 1.06 mmol) and 3-methoxy-5,6,7,8-tetrahydronaphthalene-2-sulfonyl chloride (Intermediate D) (230 mg, 0.882 mmol) afforded 5a (165 mg, 40%) as a white solid, after purification by reverse-phase preparative HPLC. ¹H NMR (400 MHz, CHLOROFORM-d) δ 7.70 (s, 1H), 7.54 (s, 1H), 7.42 (s, 1H), 6.72 (s, 1H), 3.99 (d, J=1.7 Hz, 6H), 2.76 (br t, J=5.3 Hz, 2H), 2.62 (br t, J=5.0 Hz, 2H), 1.83-1.68 (m, 5H).

3-Methoxy-N-{5-methoxy-6-[(1H-pyrazol-3-yl)amino]-1,2-benzoxazol-3-yl}-5,6,7,8-tetrahydronaphthalene-2-sulfonamide (Example 05): By the same method used to synthesize Example 03, but using 2-(dicyclohexylphosphino) 3,6-dimethoxy-2′,4′,6′-triisopropyl-1,1′-biphenyl (BrettPhos) in the place of Xantphos, N-(6-bromo-5-methoxy-1,2-benzoxazol-3-yl)-3-methoxy-5,6,7,8-tetrahydronaphthalene-2-sulfonamide (5a) (165 mg, 0.353 mmol) and tert-butyl 3-amino-1H-pyrazole-1-carboxylate (129 mg, 0.706 mmol) were reacted to give Example 05 (15 mg, 9%) as a white solid. LCMS m/z 470.1 [M+H]⁺; ¹H NMR (400 MHZ, DMSO-d₆)· 12.19 (br s, 1H), 11.19 (br s, 1H), 8.25 (s, 1H), 8.16 (br d, J=5.5 Hz, 1H), 7.59 (d, J=2.3 Hz, 1H), 7.36 (s, 1H) 7.53 (s, 1H), 6.84 (s, 1H), 6.11 (d, J=2.3 Hz, 1H), 3.90 (s, 3H), 3.76 (s, 3H), 2.76-2.65 (m, 4H), 1.70 (br s, 4H).

### Method D

### Example 06:2-Methoxy-N-{5-methoxy-6-[(3-methyl-1H-pyrazol-5-yl)amino]-1,2-benzoxazol-3-yl}benzene-1-sulfonamide

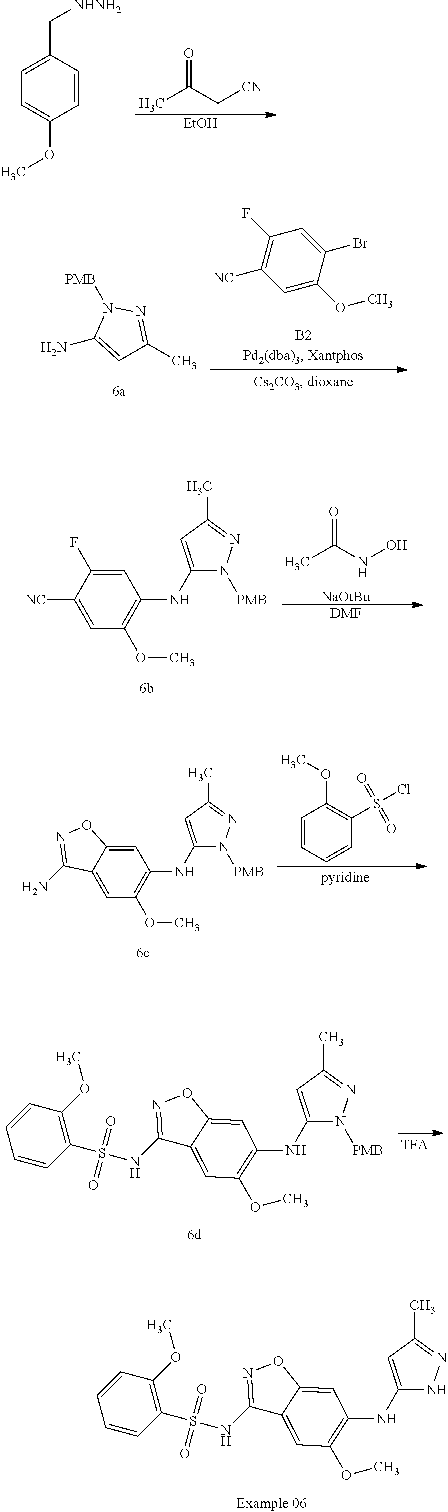

Step 1:1-[(4-Methoxyphenyl)methyl]-3-methyl-1H-pyrazol-5-amine (6a): A solution of 3-oxobutanenitrile (5.0 g, 60.2 mmol) and [(4-methoxyphenyl)methyl]hydrazine (11.9 g, 78.2 mmol) in ethanol (50 mL) was stirred at 50° C. for three hrs. The crude reaction mixture was concentrated under reduced pressure and diluted with EtOAc (50 mL). The organic layer was washed with saturated sodium bicarbonate (10 mL), H₂O (40 mL), brine, dried by anhydrous Na₂SO₄ and filtered. The filtrate was concentrated and the resultant residue was purified over silica gel (Combi-flash, 40 g) and eluted with 0-60% EtOAc-Pet. ether to afford 6a as a white solid (4 g, 49% yield). LCMS m/z 218 [M+H]⁺; ¹H NMR (400 MHZ, DMSO-d₆) δ 7.09 (d, J=8.6 Hz, 2H), 6.89-6.81 (m, 2H), 5.08 (d, J=8.1 Hz, 3H), 4.91 (s, 2H), 3.74-3.62 (m, 3H), 1.94 (s, 3H).

Step 2:2-Fluoro-5-methoxy-4-({1-[(4-methoxyphenyl)methyl]-3-methyl-1H-pyrazol-5-yl}amino) benzonitrile (6b): A yellow suspension of 4-bromo-2-fluoro-5-methoxybenzonitrile (B2) (100 mg, 0.4 mmol), 1-[(4-methoxyphenyl)methyl]-3-methyl-1H-pyrazol-5-amine (6a) (115 mg, 0.5 mmol), and cesium carbonate (425 mg, 1.3 mmol) in dioxane (6 mL) was degassed and purged with argon, followed by the addition of Pd₂(dba)₃ (40 mg, 0.04 mmol) and Xantphos (75 mg, 0.13 mmol). LCMS provided mostly product after heating the mixture at 80° C. for 18 hrs. The process was repeated using 4-bromo-2-fluoro-5-methoxybenzonitrile (B2) (1.5 g, 6.6 mmol), 1-[(4-methoxyphenyl)methyl]-3-methyl-1H-pyrazol-5-amine (6a) (1.7 g, 8.0 mmol), cesium carbonate (6.5 g, 20 mmol), Pd₂(dba)₃ (609 mg, 0.7 mmol), and Xantphos (1.1 g, 2.0 mmol) in dioxane (30 mL). After 18 hrs at 80° C., the crude reaction mixture was cooled to rt and diluted with EtOAc (50 mL) and filtered. The filtrates from both reactions were combined and purified over silica gel (Combi-flash, 40 g) and eluted with 0-45% EtOAc-Pet. ether to afford a yellow solid 6b (3 g, 56% pure by LCMS). LCMS m/z 367 [M+H]⁺.

Step 3:5-Methoxy-N⁶-{1-[(4-methoxyphenyl)methyl]-3-methyl-1H-pyrazol-5-yl}-1,2-benzoxazole-3,6-diamine (6c): A solution of N-hydroxyacetamide (1.8 g, 25 mmol) and sodium tert-butoxide (2.8 g, 25 mmol) in DMF (75 mL) was stirred for 30 mins. To the resultant white mixture was added a solution of 2-fluoro-5-methoxy-4-({1-[(4-methoxyphenyl)methyl]-3-methyl-1H-pyrazol-5-yl}amino) benzonitrile (6b) (3.0 g, 8.2 mmol) in DMF (5 mL). The mixture was stirred at 80° C. for 16 hrs. LCMS gave starting material and product. Another portion of N-hydroxyacetamide (1.8 g, 25 mmol) and sodium tert-butoxide (2.8 g, 25 mmol) was added to the crude reaction mixture. The resultant mixture was stirred at 80° C. for 16 hrs. LCMS gave more product but starting material remained. The process of adding more N-hydroxyacetamide (1.8 g, 25 mmol) and sodium tert-butoxide (2.8 g, 25 mmol) to the crude reaction mixture, followed by heating at 80° C. for 16 hrs, was repeated three more times. The crude reaction mixture was concentrated to remove DMF and the mixture was diluted with EtOAc (250 mL). The organic layer was washed with H₂O (250 mL) and the aqueous layer was extracted with EtOAc (3×250 mL). The combined organic extracts were washed with brine, dried over Na₂SO₄ and filtered. The filtrate was concentrated and the crude product was purified over silica gel (Combi-flash, 40 g) and eluted with 0-60% EtOAc-Pet. ether, which gave compound 6c as a brown oil (2 g, 64%). LCMS m/z 380 [M+H]⁺.

Step 4:2-Methoxy-N-[5-methoxy-6-({1-[(4-methoxyphenyl)methyl]-3-methyl-1H-pyrazol-5-yl}amino)-1,2-benzoxazol-3-yl]benzene-1-sulfonamide (6d): A solution of 5-methoxy-N6-{1-[(4-methoxyphenyl)methyl]-3-methyl-1H-pyrazol-5-yl}-1,2-benzoxazole-3,6-diamine (6c) (200 mg, 0.5 mmol) and 2-methoxybenzene-1-sulfonyl chloride (163 mg, 0.79 mmol) in pyridine (2 mL) was heated at 120° C. for six hrs. The crude reaction mixture was concentrated and purified over silica gel (12 g) and eluted with 0-80% EtOAc-Pet. ether, which gave compound 6d as a brown oil (50 mg, 17%). LCMS m/z 550 [M+H]⁺.

Step 5:2-Methoxy-N-{5-methoxy-6-[(3-methyl-1H-pyrazol-5-yl)amino]-1,2-benzoxazol-3-yl}benzene-1-sulfonamide (Example 06): A solution of 2-methoxy-N-[5-methoxy-6-({1-[(4-methoxyphenyl)methyl]-3-methyl-1H-pyrazol-5-yl}amino)-1,2-benzoxazol-3-yl]benzene-1-sulfonamide (6d) in TFA (2 mL) was stirred at 80° C. for 16 hrs. The crude reaction mixture was concentrated and the residue diluted with methanol (5 mL) and saturated sodium bicarbonate (1 mL). The mixture was concentrated and dissolved in DMSO (3 mL) and the crude product purified by reverse phase chromatography using a Phenomenex C18 column (75×30 mm; 3 micron) and eluted with aq ammonium hydroxide-ACN, which gave Example 06 as a white solid (6 mg, 25%). LCMS m/z 430.2 [M+H]⁺; ¹H NMR (400 MHZ, METHANOL-d₄) δ 7.89 (dd, J=1.6, 7.8 Hz, 1H), 7.65-7.52 (m, 2H), 7.26 (s, 1H), 7.13 (d, J=8.4 Hz, 1H), 7.03 (t, J=7.6 Hz, 1H), 5.88 (s, 1H), 3.97 (s, 3H), 3.87 (s, 3H), 2.27 (s, 3H).

### Method E

### Example 07: N-{6-[(3-cyclopropyl-1H-pyrazol-5-yl)amino]-5-methoxy-1,2-benzoxazol-3-yl}-2,6-dimethoxy-4-(pyridin-3-yl)benzene-1-sulfonamide

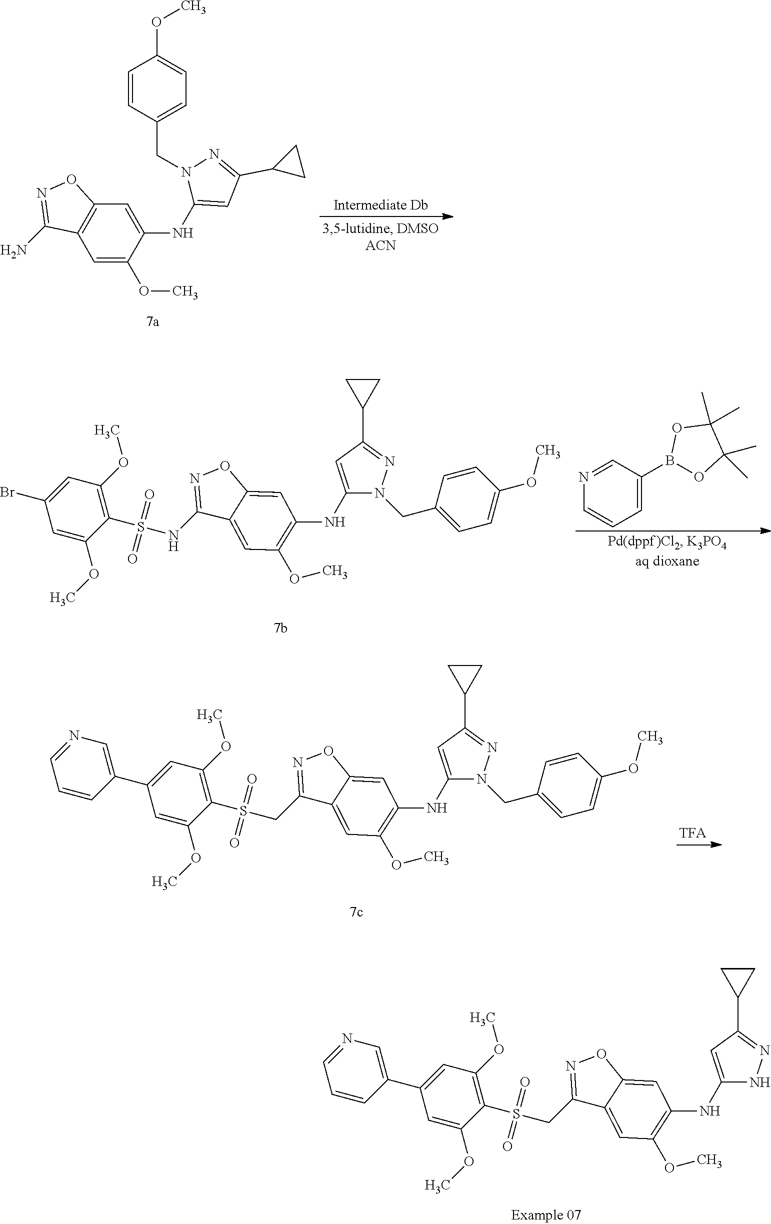 [SMILES: COc1ccc(Cn2nc(C3CC3)cc2Nc2cc3onc(N)c3cc2OC)cc1 ; COc1ccc(Cn2nc(C3CC3)cc2Nc2cc3onc(NS(=O)(=O)c4c(OC)cc(Br)cc4OC)c3cc2OC)cc1 ; CC1(C)OB(c2cccnc2)OC1(C)C ; COc1ccc(Cn2nc(C3CC3)cc2Nc2cc3onc(CS(=O)(=O)c4c(OC)cc(-c5cccnc5)cc4OC)c3cc2OC)cc1 ; COc1cc2c(CS(=O)(=O)c3c(OC)cc(-c4cccnc4)cc3OC)noc2cc1Nc1cc(C2CC2)n[nH]1]

### Step 1: Synthesis of N6-{3-cyclopropyl-1-[(4-methoxyphenyl)methyl]-1H-pyrazol-5-yl}-5-methoxy-1,2-benzoxazole-3,6-diamine (7a)

7a was prepared in a similar manner as 6c using 3-cyclopropyl-3-oxopropanenitrile in place of 3-oxobutanenitrile in the first step. ¹H NMR (400 MHZ, DMSO-d₆) 0 ppm 0.63-0.68 (m, 2H) 0.82-0.86 (m, 2H) 1.81-1.88 (m, 1H) 3.66-3.70 (m, 3H) 3.87 (s, 3H) 5.05 (s, 2H) 5.87-5.92 (m, 1H) 6.02-6.08 (m, 2H) 6.39-6.42 (m, 1H) 6.81 (d, J=8.63 Hz, 2H) 7.04 (d, J=8.63 Hz, 2H) 7.26 (s, 1H) 7.51 (s, 1H); LCMS m/z 406.1 (M+H).

### Step 2: Synthesis of 4-bromo-N-[6-({3-cyclopropyl-1-[(4-methoxyphenyl)methyl]-1H pyrazol-5-yl}amino)-5-methoxy-1,2-benzoxazol-3-yl]-2,6-dimethoxybenzene-1-sulfonamide (7b)

To a solution of N6-{3-cyclopropyl-1-[(4-methoxyphenyl)methyl]-1H-pyrazol-5-yl}-5-methoxy-1,2-benzoxazole-3,6-diamine 7a (900 mg, 2.22 mmol) and 4-bromo-2,6-dimethoxybenzene-1-sulfonyl chloride (Intermediate Db) (1.1 g, 3.6 mmol) in ACN (5 mL) was added 0.05 N DMSO in ACN (9 mg, 0.1 mmol) and 3,5-lutidine (714 mg, 6.66 mmol). The suspension was stirred at 60° C. for 16 hrs. The solvent color changed from yellow to red. The solvent was removed under reduced pressure and the resultant residue was purified over silica gel and eluted with 0-54% EtOAc in Pet. ether, which afforded 7a as a pink solid (810 mg, 53% yield). LCMS m/z 685 (M+H).

### Step 3: Synthesis of N-[6-({3-cyclopropyl-1-[(4-methoxyphenyl)methyl]-1H-pyrazol-5-yl}amino)-5-methoxy-1,2-benzoxazol-3-yl]-2,6-dimethoxy-4-(pyridin-3-yl)benzene-1-sulfonamide (7c)

To a solution of 4-bromo-N-[6-({3-cyclopropyl-1-[(4-methoxyphenyl)methyl]-1H-pyrazol-5-yl}amino)-5-methoxy-1,2-benzoxazol-3-yl]-2,6-dimethoxybenzene-1-sulfonamide (7b) (100 mg, 0.15 mmol) and 3-(4,4,5,5-tetramethyl-1,3,2-dioxaborolan-2-yl)pyridine (60 mg, 0.29 mmol) in dioxane (2.0 mL) and H₂O (0.7 mL) was added tripotassium phosphate (93 mg, 0.44 mmol) and Pd(dppf)Cl₂ (2.4 mg, 0.003 mmol). The mixture was heated to stir at 80° C. under nitrogen for 1 hour. LCMS showed the reaction was completed and the desired product was observed. The crude reaction mixture was combined with the batch from another experiment (25 mg of aryl bromide was used) and diluted with H₂O (10 mL). The aqueous layer was extracted with EtOAc (10×3 mL), and the combined organic extracts were washed with brine, dried over Na₂SO₄, filtered and concentrated to afford 7c as a brown solid (158 mg). LCMS m/z 683 [M+H]⁺.

### Step 4: Synthesis of N-{6-[(3-cyclopropyl-1H-pyrazol-5-yl)amino]-5-methoxy-1,2-benzoxazol-3-yl}-2,6-dimethoxy-4-(pyridin-3-yl)benzene-1-sulfonamide (Example 07)

The crude product 7c was dissolved in TFA (5 mL) and stirred at 80° C. for 18 hrs. LCMS showed the starting material 7c was consumed and the desired product was observed. The crude reaction mixture was concentrated under reduced pressure and diluted with H₂O. The aqueous layer was extracted with EtOAc (50 mL×2) and the combined extracts were washed with sat. aq NaHCO₃ (15 mL), brine (30 mL), dried over Na₂SO₄, and concentrated under reduced pressure to give the crude product as a brown solid (150 mg). The crude product was diluted into DMF (2 mL) and purified by reverse phase chromatography using an Phenomenex Gemini NX column (150×20 mm, 5 micron) and eluted with an aq ammonium hydroxide buffered solution and ACN gradient at 60 mL min 1, to yield Example 07 as a white solid (31 mg). LCMS m/z 563.3 [M+H]⁺; ¹H NMR (400 MHZ, DMSO-d₆) δ 11.90 (br s, 1H), 11.06 (s, 1H), 9.01 (s, 1H), 8.68-8.60 (m, 1H), 8.23-8.18 (m, 1H), 8.14 (s, 1H), 8.07 (d, J=1.1 Hz, 1H), 7.54-7.49 (m, 1H), 7.44 (s, 1H), 7.05 (d, J=2.0 Hz, 2H), 5.78 (s, 1H), 3.91-3.87 (m, 9H), 1.91-1.80 (m, 1H) 0.96-0.89 (m, 2H), 0.69-0.63 (m, 2H).

### Method F

### Example 08: N-{6-[(3-cyclopropyl-1H-pyrazol-5-yl)amino]-5-methoxy-1,2-benzoxazol-3-yl}-2,6-dimethoxy-4-(1,3-oxazol-2-yl)benzene-1-sulfonamide

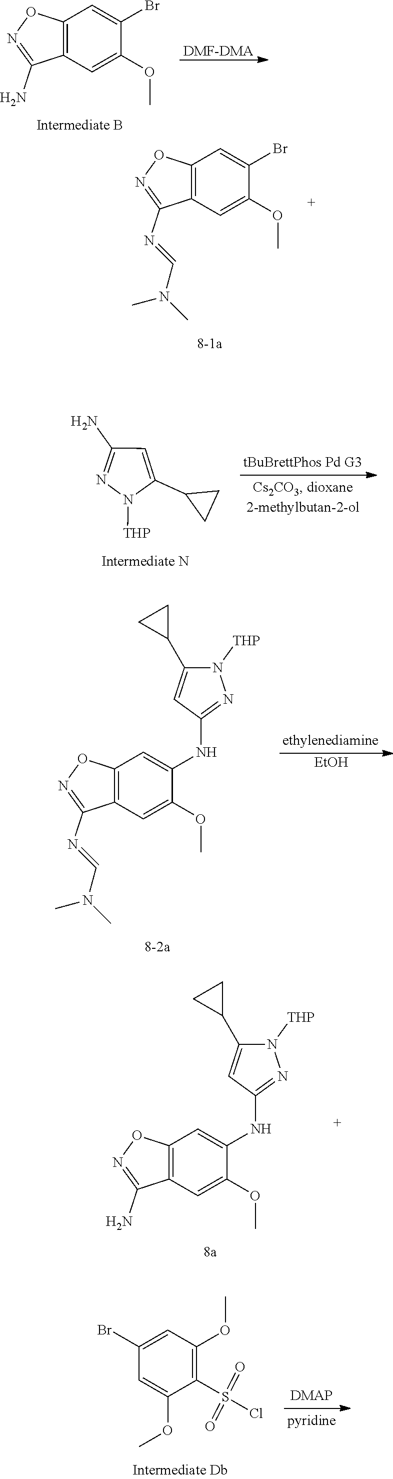

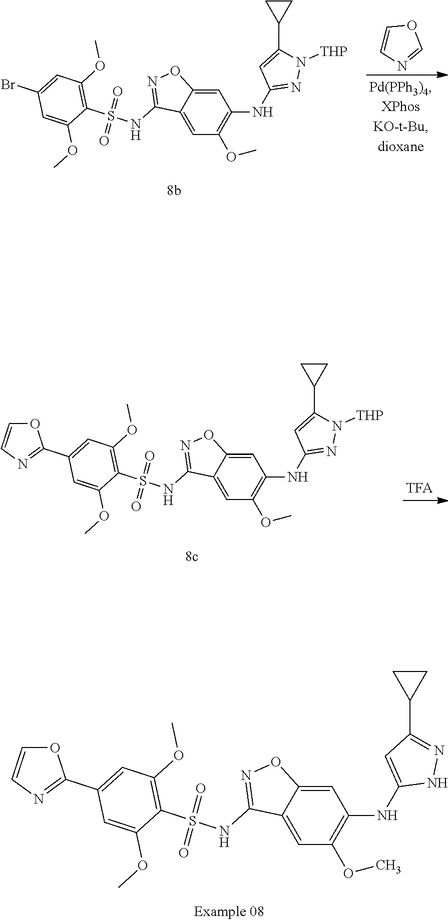

### Step 1: N-(6-bromo-5-methoxy-1,2-benzoxazol-3-yl)-N,N-dimethylmethanimidamide (8-1a)

A solution of Intermediate B (125 g, 514.28 mmol) in N,N-dimethylformamide dimethyl acetal (462.69 g, 3.88 mol, 515.82 mL) was heated to 90° C. for 30 min. To the crude reaction was added H₂O (3 L) at 15° C. ˜25° C., and the mixture was stirred for 2 hrs at 25° C., then filtered and washed with H₂O (500 mL×6). The filter cake was dried at 50° C. for 24 hrs to give 8-1a (151.2 g, 98% yield) as an off-white solid. ¹H NMR (400 MHZ, CDCl₃) δ 8.24 (s, 1H), 7.67 (s, 1H), 7.12 (s, 1H), 3.95 (s, 3H), 3.16 (s, 3H), 3.12 (s, 3H); LCMS m/z 299.8 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-N,N-dimethylmethanimidamide (8-2a)

The reaction was run in 2 batches. To a mixture of 8-1a (80 g, 268.33 mmol), tBuBrettPhos Pd G3 (27.51 g, 32.20 mmol) and Cs₂CO₃ (122.40 g, 375.67 mmol) in 2-methylbutan-2-ol (2.2 L) was added a solution of Intermediate N (66.74 g, 322.00 mmol) in dioxane (160 mL) at 25° C. under nitrogen. The reaction mixture was heated to 100° C. for 16 hrs then diluted with water (3 L) and brine (1 L) then extracted with EtOAc (3 L×3). The combined organic layers from two batches were washed with brine (1 L×2), dried over MgSO₄, filtered and concentrated. The crude material was purified by column chromatography on silica gel (0-85% EtOAc/DCM) to give 8-2a (210 g, 66% yield) as a yellow solid. ¹H NMR (400 MHZ, CDCl₃) δ 8.21 (s, 1H), 7.79 (s, 1H), 7.00-6.88 (m, 2H), 5.69 (s, 1H), 5.37-5.24 (m, 1H), 3.98-3.86 (m, 3H), 3.70-3.55 (m, 2H), 3.14 (s, 3H), 3.08 (s, 3H), 2.64-2.29 (m, 2H), 2.15 (br dd, J=3.7, 9.6 Hz, 1H), 1.79-1.61 (m, 4H), 0.99-0.90 (m, 2H), 0.81-0.45 (m, 2H); LCMS m/z 425.2 (M+H)⁺.

### Step 3: Synthesis of N6-[5-cyclopropyl-1-(oxan-2-yl)-1H-pyrazol-3-yl]-5-methoxy-1,2-benzoxazole-3,6-diamine (8a)

Reaction was run in 2 batches. To a solution of 8-2a (105 g, 247.35 mmol) in EtOH (2.6 L) was added ethane-1,2-diamine (93.17 g, 1.55 mol, 103.75 mL) at 25° C. The reaction was heated to 80° C. for 16 hrs then cooled to 25° C. and a ˜2:1 mixture of water/EtOH (4.5 L) was added dropwise. The reaction mixture was stirred for 30 mins, then filtered and washed with H₂O (900 mL×2). The filter cake from two batches was dried in oven at 50° C. for 18 hrs to give crude 8a. The crude solid was purified by column chromatography on silica gel (0-85% EtOAc/DCM). The resulting material was suspended in MTBE (1.5 L) and stirred at 25° C. for 2 hrs, then filtered, rinsed and dried to give 8a (101.3 g, 55% yield) as a yellow solid. ¹H NMR (400 MHZ, CDCl₃) δ 7.81 (s, 1H), 6.94 (s, 1H), 6.74 (s, 1H), 5.68 (s, 1H), 5.48 (dd, J=2.4, 10.0 Hz, 1H), 4.27-4.14 (m, 2H), 4.09 (br d, J=11.4 Hz, 1H), 3.89 (s, 3H), 3.72-3.63 (m, 1H), 2.66-2.44 (m, 1H), 2.15 (br dd, J=3.5, 9.1 Hz, 1H), 1.97 (br dd, J=2.1, 13.5 Hz, 1H), 1.91-1.83 (m, 1H), 1.82-1.68 (m, 2H), 1.59 (br d, J=11.5 Hz, 1H), 0.98 (dd, J=2.0, 8.4 Hz, 2H), 0.86-0.76 (m, 1H), 0.69-0.58 (m, 1H); LCMS m/z 370.2 (M+H)⁺.

### Step 4: Synthesis of 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide (8b)

To a solution of 8a (40 g, 108.28 mmol) in pyridine (200 mL) was added Intermediate Db (41.11 g, 130.26 mmol) and DMAP (1.59 g, 12.99 mmol) at 25° C. under nitrogen. The reaction solution was stirred at 25° C. for 16 hrs. The crude reaction mixture was combined with another crude reaction mixture that used 4 g of 8a. Both reaction mixtures were diluted with DCM (1.6 L) and washed with 1M AcOH (2.8 L) then extracted with DCM (900 mL×3). The combined organic phases were washed with brine (500 mL×2), then dried over MgSO₄, filtered and concentrated. The crude residue was purified by column chromatography on silica gel (0-8% EtOAc/DCM) to give 8b (68.6 g, 87% yield) as off-white solid. ¹H NMR (400 MHZ, DMSO-d₆) δ 11.12 (s, 1H), 8.38-8.08 (m, 2H), 7.53-7.31 (m, 1H), 6.99 (s, 2H), 5.77 (d, J=8.8 Hz, 1H), 5.50 (dd, J=2.3, 9.7 Hz, 1H), 3.93 (br d, J=11.0 Hz, 1H), 3.89 (s, 3H), 3.79 (s, 6H), 3.70-3.53 (m, 1H), 2.41-2.26 (m, 1H), 2.10-2.01 (m, 1H), 1.95-1.89 (m, 2H), 1.80-1.64 (m, 1H), 1.63-1.46 (m, 2H), 0.97 (dd, J=2.4, 8.3 Hz, 2H), 0.77-0.53 (m, 2H); LCMS m/z 648.1, 650.1 (M+H)⁺.

### Step 5: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-(1,3-oxazol-2-yl)benzene-1-sulfonamide (8c)

To a mixture of 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide 8b (280 mg, 0.43 mmol) in dioxane (6.0 mL) was added 1,3-oxazole (60 mg, 0.86 mmol), potassium tert-butoxide (104 mg, 0.93 mmol), 2-dicyclohexylphosphino-2′,4′,6′-triisopropylbiphenyl (41 mg, 0.09 mmol) and tetrakis(triphenylphosphine)palladium(0) (75 mg, 0.06 mmol) at 20° C. under nitrogen. Then the mixture was stirred at 100° C. for 10 hrs under nitrogen. LCMS of the crude reaction mixture showed the starting material was consumed and the desired product mass was observed. The crude reaction mixture was combined with another batch (20 mg of aryl bromide was used) and the mixture was filtered. The filter cake was washed with EtOAc (2×30 mL) and purified by combi-flash (12 g silica gel, Pet. ether/EtOAc, 0˜100%, then 0-20% methanol/DCM) to afford N-(6-{[5-cyclopropyl-1-(oxan-2-yl)-1H-pyrazol-3-yl]amino}-5-methoxy-1,2-benzoxazol-3-yl)-2,6-dimethoxy-4-(1,3-oxazol-2-yl)benzene-1-sulfonamide 8c (140 mg) as yellow gum. LCMS m/z 637 [M+H]⁺.

### Step 6: Synthesis of N-{6-[(3-cyclopropyl-1H-pyrazol-5-yl)amino]-5-methoxy-1,2-benzoxazol-3-yl}-2,6-dimethoxy-4-(1,3-oxazol-2-yl)benzene-1-sulfonamide (Example 8)

A solution of 8c (140 mg, 0.22 mmol) in DCM (2.0 mL) and TFA (5.0 mL) was stirred at rt for 3 hrs. LCMS showed the desired product was formed. The crude reaction mixture was concentrated under reduced pressure to give the crude product (200 mg), which was dissolved in DMF (2 mL) and purified by C18 reverse phase chromatography and provided Example 08 as a white solid (16 mg). LCMS m/z 553.2 [M+H]⁺; ¹H NMR (400 MHZ, DMSO-d₆) δ 11.91 (br s, 1H), 11.17 (br s, 1H), 8.32 (s, 1H), 8.14 (s, 1H), 8.08 (br s, 1H), 7.46 (s, 1H), 7.40 (s, 1H), 7.26 (s, 2H), 5.78 (s, 1H), 3.89 (s, 3H), 3.87 (s, 6H), 1.90-1.79 (m, 1H), 0.96-0.87 (m, 2H), 0.70-0.63 (m, 2H).

The compounds in Table 18 below were prepared according to the methods A-F as described above for Examples 01-08, using Intermediates A-N or commercially available reagents. The following examples were synthesized with non-critical changes or substitutions to the exemplified procedures that will be apparent to those skilled in the art.

TABLE 18

| Example | | | |
|---|---|---|---|
| Number | Structure/IUPAC Name | Analytical Data | Method |
| 09 | 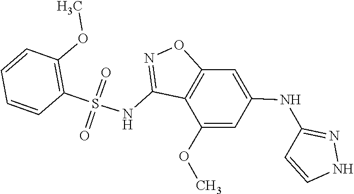 [SMILES: COc1ccccc1S(=O)(=O)Nc1noc2cc(Nc3cc[nH]n3)cc(OC)c12] | LCMS m/z 416.1 [M + H]⁺; ¹H NMR (400 MHz, DMSO-d₆) δ 12.20 (br s, 1H), 9.11 (s, 1H), 7.81 (d, J = 6.8 Hz, 1H), 7.67- 7.54 (m, 2H), 7.35 (s, 1H), 7.27 (s, 1H), 7.20 (d, J = 8.5 Hz, 1H), 7.09 (t, J = 7.5 Hz, 1H), 6.60 (s, 1H), 5.89 (s, 1H), 3.82 (s, 6H) | C |
| | 2-methoxy-N-{4-methoxy-6-[(1H- | | |
| | pyrazol-3-yl)amino]-1,2- | | |
| | benzoxazol-3-yl}benzene-1- | | |
| | sulfonamide | | |
| 10 | 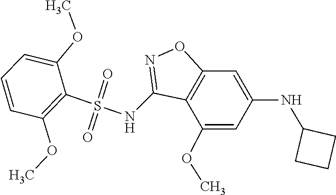 [SMILES: COc1cccc(OC)c1S(=O)(=O)Nc1noc2cc(NC3CCC3)cc(OC)c12] | LCMS m/z 434.1 [M + H]⁺; ¹H NMR (400 MHz, DMSO-d₆) δ 7.49 (t, J = 8.4 Hz, 1H), 6.81-6.75 (m, 3H), 6.05 (s, 1H), 5.95 (s, 1H), 3.90-3.81 (m, 4H), 3.78 (s, 6H), 2.40-2.29 (m, 2H), 1.88- 1.65 (m, 4H) | A |
| | N-[6-(cyclobutylamino)-4- | | |
| | methoxy-1,2-benzoxazol-3-yl]-2,6- | | |
| | dimethoxybenzene-1-sulfonamide | | |
| 11 | 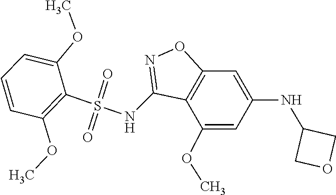 [SMILES: COc1cccc(OC)c1S(=O)(=O)Nc1noc2cc(NC3COC3)cc(OC)c12] | LCMS m/z 436.1 [M + H]⁺; ¹H NMR (400 MHz, DMSO-d₆) δ 9.06 (br s, 1H), 7.48 (t, J = 8.5 Hz, 1H), 7.21 (br d, J = 6.0 Hz, 1H), 6.77 (d, J = 8.5 Hz, 2H), 6.08 (s, 1H), 5.94 (s, 1H), 4.86 (t, J = 6.5 Hz, 2H), 4.63-4.55 (m, 1H), 4.40 (t, J = 6.0 Hz, 2H), 3.85 (s, 3H), 3.78 (s, 6H) | A |
| | 2,6-dimethoxy-N-{4-methoxy-6- | | |
| | [(oxetan-3-yl)amino]-1,2- | | |
| | benzoxazol-3-yl}benzene-1- | | |
| | sulfonamide | | |
| 12 | 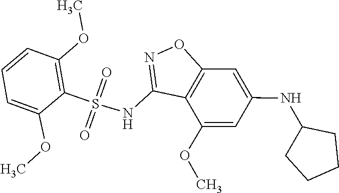 [SMILES: COc1cccc(OC)c1S(=O)(=O)Nc1noc2cc(NC3CCCC3)cc(OC)c12] | LCMS m/z 448.1 [M + H]⁺; ¹H NMR (400 MHz, DMSO-d₆) δ 8.94 (br s, 1H), 7.46 (br t, J = 7.7 Hz, 1H), 6.75 (br d, J = 8.4 Hz, 2H), 6.42 (br s, 1H), 6.09 (s, 1H), 6.01 (s, 1H), 3.83 (s, 3H), 3.77 (s, 6H), 3.75-3.67 (m, 1H), 1.92 (qd, J = 6.3, 12.3 Hz, 2H), 1.72-1.60 (m, 2H), 1.60-1.49 (m, 2H), 1.48-1.38 (m, 2H) | A |
| | N-[6-(cyclopentylamino)-4- | | |
| | methoxy-1,2-benzoxazol-3-yl]-2,6- | | |
| | dimethoxybenzene-1-sulfonamide | | |
| 13 | 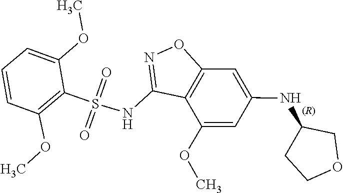 | LCMS m/z 450.1 [M + H]⁺; ¹H NMR (400 MHz, DMSO-d₆) δ 7.49 (t, J = 8.5 Hz, 1H), 6.77 (d, J = 8.5 Hz, 2H), 6.71 (br d, J = 6.3 Hz, 1H), 6.10 (d, J = 15.8 Hz, 2H), 4.01 (br s, 1H), 3.89-3.85 (m, 1H), 3.84 (s, 3H), 3.80 (d, J = 8.3 Hz, 1H), 3.78 (s, 6H), 3.71 (dt, J = 5.5, 8.3 Hz, 1H), 3.52 (dd, J = 3.4, 8.9 Hz, 1H), 2.24-2.13 (m, 1H), 1.80-1.70 (m, 1H) | A |
| | 2,6-dimethoxy-N-(4-methoxy-6- | | |
| | {[(3R)-oxolan-3-yl]amino}-1,2- | | |
| | benzoxazol-3-yl)benzene-1- | | |
| | sulfonamide | | |
| 14 | 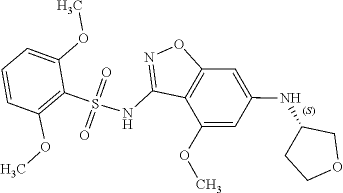 | LCMS m/z 450.1 [M + H]⁺; ¹H NMR (400 MHz, DMSO-d₆) δ 7.48 (t, J = 8.5 Hz, 1H), 6.77 (d, J = 8.6 Hz, 2H), 6.71 (d, J = 6.4 Hz, 1H), 6.15-6.03 (m, 2H), 4.05-3.97 (m, 1H), 3.89-3.85 (m, 1H), 3.83 (s, 3H), 3.80 (d, J = 8.1 Hz, 1H), 3.78 (s, 6H), 3.71 (dt, J = 5.5, 8.2 Hz, 1H), 3.52 (dd, J = 3.4, 8.9 Hz, 1H), 2.24-2.12 (m, 1H), 1.82-1.68 (m, 1H) | A |
| | 2,6-dimethoxy-N-(4-methoxy-6- | | |
| | {[(3S)-oxolan-3-yl]amino}-1,2- | | |
| | benzoxazol-3-yl)benzene-1- | | |
| | sulfonamide | | |
| 15 | 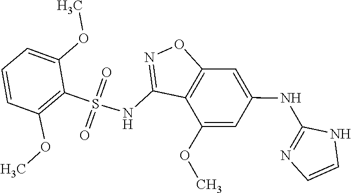 [SMILES: COc1cccc(OC)c1S(=O)(=O)Nc1noc2cc(Nc3ncc[nH]3)cc(OC)c12] | LCMS m/z 446.1 [M + H]⁺; ¹H NMR (400 MHz, DMSO-d₆) δ 9.35 (br s, 1H), 7.48 (t, J = 8.4 Hz, 1H), 7.37 (s, 1H), 6.79-6.67 (m, 5H), 3.86 (s, 3H), 3.77 (s, 6H) | A |
| | N-{6-[(1H-imidazol-2-yl)amino]-4- | | |
| | methoxy-1,2-benzoxazol-3-yl}-2,6- | | |
| | dimethoxybenzene-1-sulfonamide | | |
| 16 | 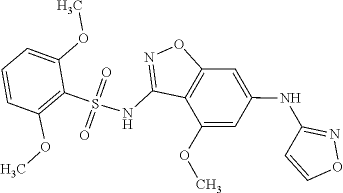 [SMILES: COc1cccc(OC)c1S(=O)(=O)Nc1noc2cc(Nc3ccon3)cc(OC)c12] | LCMS m/z 447.1 [M + H]⁺; ¹H NMR (400 MHz, DMSO-d₆) δ 9.80 (s, 1H), 8.69 (d, J = 1.8 Hz, 1H), 7.50 (t, J = 8.4 Hz, 1H), 7.34 (d, J = 1.3 Hz, 1H), 6.78 (d, J = 8.5 Hz, 2H), 6.72 (d, J = 1.1 Hz, 1H), 6.29 (d, J = 1.8 Hz, 1H), 3.91 (s, 3H), 3.78 (s, 6H) | A |
| | 2,6-dimethoxy-N-{4-methoxy-6- | | |
| | [(1,2-oxazol-3-yl)amino]-1,2- | | |
| | benzoxazol-3-yl}benzene-1- | | |
| | sulfonamide | | |
| 17 | 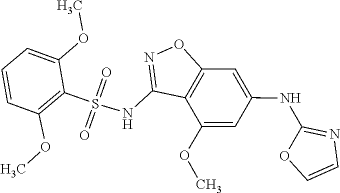 [SMILES: COc1cccc(OC)c1S(=O)(=O)Nc1noc2cc(Nc3ncco3)cc(OC)c12] | LCMS m/z 447.1 [M + H]⁺; ¹H NMR (400 MHz, DMSO-d₆) δ 7.69 (d, J = 1.0 Hz, 1H), 7.44 (d, J = 1.2 Hz, 1H), 7.35 (t, J = 8.4 Hz, 1H), 7.04 (d, J = 1.0 Hz, 1H), 6.83 (d, J = 1.3 Hz, 1H), 6.68 (d, J = 8.4 Hz, 2H), 3.86 (s, 3H), 3.68 (s, 6H) | A |
| | 2,6-dimethoxy-N-{4-methoxy-6- | | |
| | [(1,3-oxazol-2-yl)amino]-1,2- | | |
| | benzoxazol-3-yl}benzene-1- | | |
| | sulfonamide | | |
| 18 | 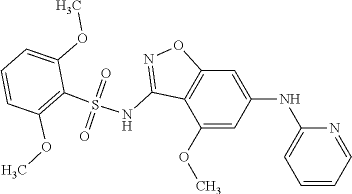 [SMILES: COc1cccc(OC)c1S(=O)(=O)Nc1noc2cc(Nc3ccccn3)cc(OC)c12] | LCMS m/z 457.1 [M + H]⁺; ¹H NMR (400 MHz, DMSO-d₆) δ 9.61 (s, 1H), 9.35 (s, 1H), 8.25 (br d, J = 3.7 Hz, 1H), 7.98 (s, 1H), 7.70-7.62 (m, 1H), 7.50 (t, J = 8.4 Hz, 1H), 6.92 (d, J = 8.4 Hz, 1H), 6.88-6.83 (m, 2H), 6.78 (d, J = 8.6 Hz, 2H), 3.92-3.90 (m, 3H), 3.79 (s, 6H) | A |
| | 2,6-dimethoxy-N-{4-methoxy-6- | | |
| | [(pyridin-2-yl)amino]-1,2- | | |
| | benzoxazol-3-yl}benzene-1- | | |
| | sulfonamide | | |
| 19 | 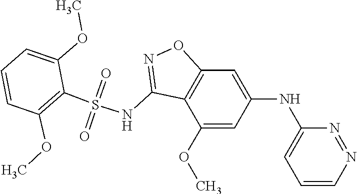 [SMILES: COc1cccc(OC)c1S(=O)(=O)Nc1noc2cc(Nc3cccnn3)cc(OC)c12] | LCMS m/z 458.0 [M + H]⁺; ¹H NMR (400 MHz, DMSO-d₆) δ 9.80 (s, 1H), 8.76 (d, J = 4.5 Hz, 1H), 8.02 (s, 1H), 7.55-7.44 (m, 2H), 7.22 (d, J = 8.8 Hz, 1H), 6.90 (s, 1H), 6.78 (d, J = 8.5 Hz, 2H), 3.91 (s, 3H), 3.78 (s, 6H) | A |
| | 2,6-dimethoxy-N-{4-methoxy-6- | | |
| | [(pyridazin-3-yl)amino]-1,2- | | |
| | benzoxazol-3-yl}benzene-1- | | |
| | sulfonamide | | |
| 20 | 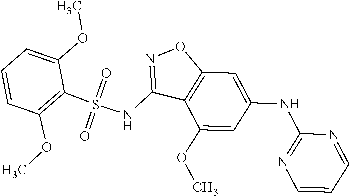 [SMILES: COc1cccc(OC)c1S(=O)(=O)Nc1noc2cc(Nc3ncccn3)cc(OC)c12] | LCMS m/z 458.1 [M + H]⁺; ¹H NMR (400 MHz, DMSO-d₆) δ 10.09 (s, 1H), 9.42 (br s, 1H), 8.59 (d, J = 4.8 Hz, 2H), 7.96 (s, 1H), 7.51 (t, J = 8.4 Hz, 1H), 7.13 (s, 1H), 6.97 (t, J = 4.9 Hz, 1H), 6.79 (d, J = 8.5 Hz, 2H), 3.90 (s, 3H), 3.79 (s, 6H) | A |
| | 2,6-dimethoxy-N-{4-methoxy-6- | | |
| | [(pyrimidin-2-yl)amino]-1,2- | | |
| | benzoxazol-3-yl}benzene-1- | | |
| | sulfonamide | | |
| 21 | 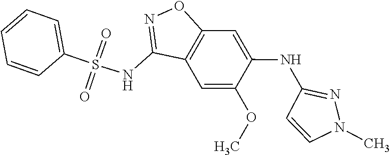 [SMILES: COc1cc2c(NS(=O)(=O)c3ccccc3)noc2cc1Nc1ccn(C)n1] | LCMS m/z 400.0 [M + H]⁺; ¹H NMR (400 MHz, DMSO-d₆) 8.28 (s, 1H), 8.18 (s, 1H), 7.95 (br d, J = 7.4 Hz, 2H), 7.71-7.64 (m, 1H), 7.64-7.58 (m, 2H), 7.53 (d, J = 2.1 Hz, 1H), 7.25 (s, 1H), 6.06 (d, J = 2.3 Hz, 1H), 3.89 (s, 3H), 3.79 (s, 3H) | B |
| | N-{5-methoxy-6-[(1-methyl-1H- | | |
| | pyrazol-3-yl)amino]-1,2- | | |
| | benzoxazol-3- | | |
| | yl}benzenesulfonamide | | |
| 22 | 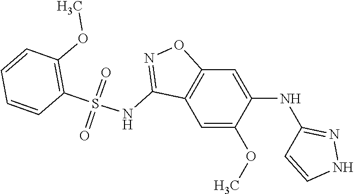 [SMILES: COc1cc2c(NS(=O)(=O)c3ccccc3OC)noc2cc1Nc1cc[nH]n1] | LCMS m/z 416.1 [M + H]⁺; ¹H NMR (400 MHz, DMSO-d₆) δ 7.94 (dd, J = 1.7, 7.8 Hz, 1H), 7.66 (br s, 1H), 7.58 (d, J = 2.2 Hz, 1H), 7.55-7.48 (m, 1H), 7.29 (s, 1H), 7.11 (d, J = 8.1 Hz, 1H), 7.06-6.96 (m, 1H), 6.13 (d, J = 2.3 Hz, 1H), 3.99 (s, 3H), 3.87 (s, 3H) | C |
| | 2-methoxy-N-{5-methoxy-6-[(1H- | | |
| | pyrazol-3-yl)amino]-1,2- | | |
| | benzoxazol-3-yl}benzene-1- | | |
| | sulfonamide | | |
| 23 | 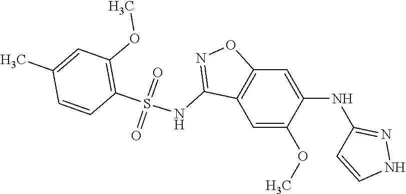 [SMILES: COc1cc2c(NS(=O)(=O)c3ccc(C)cc3OC)noc2cc1Nc1cc[nH]n1] | LCMS m/z 430.0 [M + H]⁺; ¹H NMR (400 MHz, METHANOL-d₄) δ 7.77 (d, J = 7.9 Hz, 1H), 7.70 (br s, 1H), 7.58 (d, J = 2.4 Hz, 1H), 7.30 (s, 1H), 6.97 (s, 1H), 6.87 (d, J = 7.9 Hz, 1H), 6.13 (d, J = 2.3 Hz, 1H), 4.00 (s, 3H), 3.88 (s, 3H), 2.38 (s, 3H) | C |
| | 2-methoxy-N-{5-methoxy-6-[(1H- | | |
| | pyrazol-3-yl)amino]-1,2- | | |
| | benzoxazol-3-yl}-4- | | |
| | methylbenzene-1-sulfonamide | | |
| 24 | 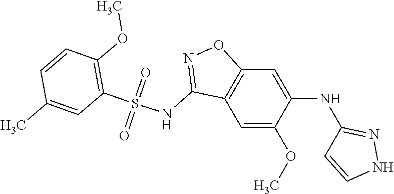 [SMILES: COc1cc2c(NS(=O)(=O)c3cc(C)ccc3OC)noc2cc1Nc1cc[nH]n1] | LCMS m/z 430.0 [M + H]⁺; ¹H NMR (400 MHz, DMSO-d₆) δ 12.19 (br s, 1H), 11.23 (br s, 1H), 8.27 (br s, 1H), 8.19 (br s, 1H), 7.67 (br s, 1H), 7.60 (br s, 1H), 7.46-7.35 (m, 2H), 7.08(br d, J = 8.4 Hz, 1H), 6.12 (s, 1H), 3.91 (s, 3H), 3.79 (s, 3H), 2.30 (s, 3H) | C |
| | 2-methoxy-N-{5-methoxy-6-[(1H- | | |
| | pyrazol-3-yl)amino]-1,2- | | |
| | benzoxazol-3-yl}-5- | | |
| | methylbenzene-1-sulfonamide | | |
| 25 | 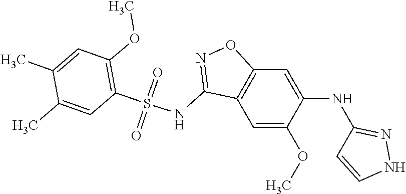 [SMILES: COc1cc2c(NS(=O)(=O)c3cc(C)c(C)cc3OC)noc2cc1Nc1cc[nH]n1] | LCMS m/z 444.1 [M + H]⁺; ¹H NMR (400 MHz, METHANOL-d₄) δ 7.72 (br s, 1H), 7.65 (s, 1H), 7.58 (d, J = 2.4 Hz, 1H), 7.29 (s, 1H), 6.94 (s, 1H), 6.13 (d, J = 2.4 Hz, 1H), 4.00 (s, 3H), 3.86 (s, 3H), 2.30 (s, 3H), 2.23 (s, 3H) | C |
| | 2-methoxy-N-{5-methoxy-6-[(1H- | | |
| | pyrazol-3-yl)amino]-1,2- | | |
| | benzoxazol-3-yl}-4,5- | | |
| | dimethylbenzene-1-sulfonamide | | |
| 26 | 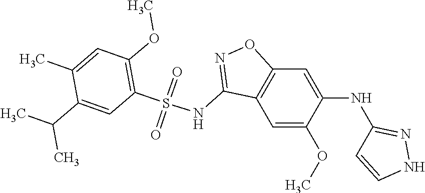 [SMILES: COc1cc2c(NS(=O)(=O)c3cc(C(C)C)c(C)cc3OC)noc2cc1Nc1cc[nH]n1] | LCMS m/z 472.0 [M + H]⁺; ¹H NMR (400 MHz, METHANOL-d₄) δ 7.73 (s, 1H), 7.71 (br s, 1H), 7.58 (d, J = 2.5 Hz, 1H), 7.33 (s, 1H), 6.91 (s, 1H), 6.13 (d, J = 2.5 Hz, 1H), 4.01 (s, 3H), 3.84 (s, 3H), 3.17-3.02 (m, 1H), 2.36 (s, 3H), 1.17 (d, J = 7.0 Hz, 6H) | C |
| | 2-methoxy-N-{5-methoxy-6-[(1H- | | |
| | pyrazol-3-yl)amino]-1,2- | | |
| | benzoxazol-3-yl}-4-methyl-5- | | |
| | (propan-2-yl)benzene-1- | | |
| | sulfonamide | | |
| 27 | 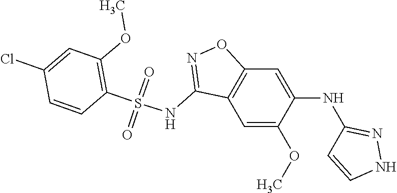 [SMILES: COc1cc2c(NS(=O)(=O)c3ccc(Cl)cc3OC)noc2cc1Nc1cc[nH]n1] | LCMS m/z 450.0 [M + H]⁺; ¹H NMR (400 MHz, DMSO-d₆) δ 12.19 (br s, 1H), 11.45 (br s, 1H), 8.27 (s, 1H), 8.19 (s, 1H), 7.86 (d, J = 8.5 Hz, 1H), 7.60 (s, 1H), 7.36-7.28 (m, 2H), 7.18 (dd, J = 1.6, 8.5 Hz, 1H), 6.12 (s, 1H), 3.91 (s, 3H), 3.86 (s, 3H) | B |
| | 4-chloro-2-methoxy-N-{5- | | |
| | methoxy-6-[(1H-pyrazol-3- | | |
| | yl)amino]-1,2-benzoxazol-3- | | |
| | yl}benzene-1-sulfonamide | | |
| 28 | 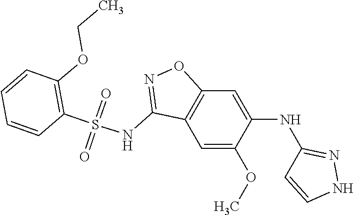 [SMILES: CCOc1ccccc1S(=O)(=O)Nc1noc2cc(Nc3cc[nH]n3)c(OC)cc12] | LCMS m/z 430.0 [M + H]⁺; ¹H NMR (400 MHz, METHANOL-d₄) δ 7.93 (br d, J = 7.9 Hz, 1H), 7.71 (br s, 1H), 7.61-7.51 (m, 2H), 7.27 (s, 1H), 7.12 (br d, J = 8.4 Hz, 1H), 7.03 (br t, J = 7.6 Hz, 1H), 6.11 (s, 1H), 4.59 (br s, 2H), 4.17 (q, J = 7.0 Hz, 2H), 3.97 (s, 3H), 1.34 (br t, J = 6.9 Hz, 3H) | C |
| | 2-ethoxy-N-{5-methoxy-6-[(1H- | | |
| | pyrazol-3-yl)amino]-1,2- | | |
| | benzoxazol-3-yl}benzene-1- | | |
| | sulfonamide | | |
| 29 | 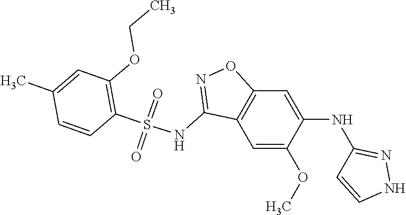 [SMILES: CCOc1cc(C)ccc1S(=O)(=O)Nc1noc2cc(Nc3cc[nH]n3)c(OC)cc12] | LCMS m/z 444.0 [M + H]⁺; ¹H NMR (400 MHz, METHANOL-d₄) δ 7.80 (d, J = 8.1 Hz, 1H), 7.71 (s, 1H), 7.58 (d, J = 2.3 Hz, 1H), 7.29 (s, 1H), 6.95 (s, 1H), 6.86 (d, J = 8.1 Hz, 1H), 6.13 (d, J = 2.3 Hz, 1H), 4.16 (q, J = 7.0 Hz, 2H), 3.99 (s, 3H), 2.37 (s, 3H), 1.36 (t, J = 7.0 Hz, 3H) | C |
| | 2-ethoxy-N-{5-methoxy-6-[(1H- | | |
| | pyrazol-3-yl)amino]-1,2- | | |
| | benzoxazol-3-yl}-4- | | |
| | methylbenzene-1-sulfonamide | | |
| 30 | 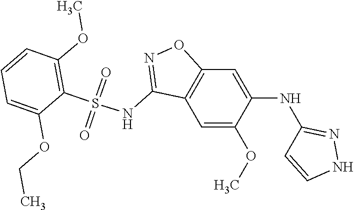 [SMILES: CCOc1cccc(OC)c1S(=O)(=O)Nc1noc2cc(Nc3cc[nH]n3)c(OC)cc12] | LCMS m/z 460.0 [M + H]⁺; ¹H NMR (400 MHz, DMSO-d₆) δ 12.19 (s, 1H), 10.94 (s, 1H), 8.25 (s, 1H), 8.19 (s, 1H), 7.62- 7.58 (m, 1H), 7.45 (t, J = 8.5 Hz, 1H), 7.41 (s, 1H), 6.75 (d, J = 4.3 Hz, 1H), 6.72 (d, J = 4.3 Hz, 1H), 6.12 (t, J = 2.2 Hz, 1H), 4.06 (q, J = 7.0 Hz, 2H), 3.88 (s, 3H), 3.76 (s, 3H), 1.21 (t, J = 7.0 Hz, 3H) | B |
| | 2-ethoxy-6-methoxy-N-{5- | | |
| | methoxy-6-[(1H-pyrazol-3- | | |
| | yl)amino]-1,2-benzoxazol-3- | | |
| | yl}benzene-1-sulfonamide | | |
| 31 | 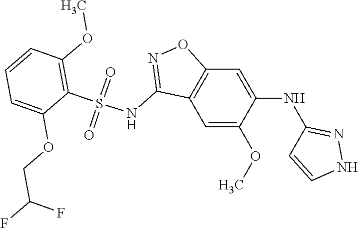 [SMILES: COc1cc2c(NS(=O)(=O)c3c(OC)cccc3OCC(F)F)noc2cc1Nc1cc[nH]n1] | LCMS m/z 496.0 [M + H]⁺; ¹H NMR (400 MHz, DMSO-d₆) δ 12.21 (s, 1H), 11.06 (s, 1H), 8.28 (s, 1H), 8.21 (s, 1H), 7.62 (t, J = 1.9 Hz, 1H), 7.52 (t, J = 8.5 Hz, 1H), 7.41 (s, 1H), 6.87 (d, J = 8.1 Hz, 1H), 6.84 (br d, J = 8.5 Hz, 1H), 6.38 (tt, J = 3.8, 54.6, Hz 1H), 6.13 (t, J = 2.1 Hz, 1H), 4.39 (dt, J = 3.8, 14.1 Hz, 2H), 3.91 (s, 3H), 3.78 (s, 3H) ¹⁹F NMR (376 MHz, DMSO-d₆) δ −124.71 (br s, 1F) | B |
| | 2-(2,2-difluoroethoxy)-6-methoxy- | | |
| | N-{5-methoxy-6-[(1H-pyrazol-3- | | |
| | yl)amino]-1,2-benzoxazol-3- | | |
| | yl}benzene-1-sulfonamide | | |
| 32 | 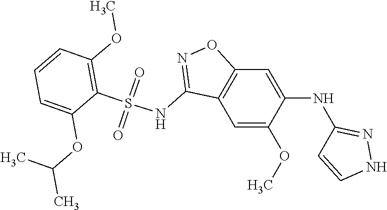 [SMILES: COc1cc2c(NS(=O)(=O)c3c(OC)cccc3OC(C)C)noc2cc1Nc1cc[nH]n1] | LCMS m/z 474.1 [M + H]⁺; ¹H NMR (400 MHz, DMSO-d₆) δ 12.19 (s, 1H), 10.87 (s, 1H), 8.25 (s, 1H), 8.20 (s, 1H), 7.60 (s, 1H), 7.47-7.38 (m, 2H), 6.75 (d, J = 8.1 Hz, 1H), 6.71 (br d, J = 8.3 Hz, 1H), 6.12 (t, J = 2.2 Hz, 1H), 4.65 (spt, J = 6.1 Hz, 1H), 3.87 (s, 3H), 3.77 (s, 3H), 1.13 (d, J = 6.0 Hz, 6H) | B |
| | 2-methoxy-N-{5-methoxy-6-[(1H- | | |
| | pyrazol-3-yl)amino]-1,2- | | |
| | benzoxazol-3-yl}-6-[(propan-2- | | |
| | yl)oxy]benzene-1-sulfonamide | | |
| 33 | 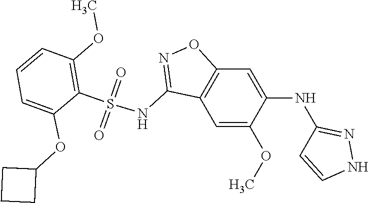 [SMILES: COc1cc2c(NS(=O)(=O)c3c(OC)cccc3OC3CCC3)noc2cc1Nc1cc[nH]n1] | LCMS m/z 486.1 [M + H]⁺; ¹H NMR (400 MHz, DMSO-d₆) δ 12.17 (br s, 1H), 10.98 (br s, 1H), 8.22 (br s, 1H), 8.18 (s, 1H), 7.59 (s, 1H), 7.47-7.32 (m, 2H), 6.71 (d, J = 8.5 Hz, 1H), 6.50 (br d, J = 8.3 Hz, 1H), 6.10 (t, J = 2.1 Hz, 1H), 4.66 (quin, J = 7.3 Hz, 1H), 3.86 (s, 3H), 3.76 (s, 3H), 2.21 (br s, 2H), 1.88 (quin, J = 9.7 Hz, 2H), 1.57-1.43 (m, 2H) | B |
| | 2-(cyclobutyloxy)-6-methoxy-N-{5- | | |
| | methoxy-6-[(1H-pyrazol-3- | | |
| | yl)amino]-1,2-benzoxazol-3- | | |
| | yl}benzene-1-sulfonamide | | |
| 34 | 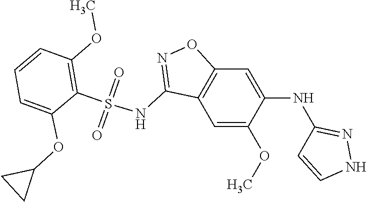 [SMILES: COc1cc2c(NS(=O)(=O)c3c(OC)cccc3OC3CC3)noc2cc1Nc1cc[nH]n1] | LCMS m/z 472.1 [M + H]⁺; ¹H NMR (400 MHz, DMSO-d₆) δ 12.19 (br s, 1H), 10.97 (s, 1H), 8.25 (s, 1H), 8.19 (s, 1H), 7.60 (s, 1H), 7.49 (t, J = 8.5 Hz, 1H), 7.39 (s, 1H), 7.02 (d, J = 8.3 Hz, 1H), 6.78 (d, J = 8.4 Hz, 1H), 6.12 (s, 1H), 3.91-3.85 (m, 4H), 3.78 (s, 3H), 0.69-0.60 (m, 2H), 0.55- 0.48 (m, 2H) | B |
| | 2-(cyclopropyloxy)-6-methoxy-N- | | |
| | {5-methoxy-6-[(1H-pyrazol-3- | | |
| | yl)amino]-1,2-benzoxazol-3- | | |
| | yl}benzene-1-sulfonamide | | |
| 35 | 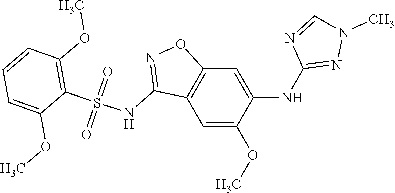 [SMILES: COc1cc2c(NS(=O)(=O)c3c(OC)cccc3OC)noc2cc1Nc1ncn(C)n1] | MS (flow injection mode) m/z 460.9 [M + H]⁺; ¹H NMR (400 MHz, DMSO-d₆) δ 11.09 (s, 1H), 8.31 (s, 1H), 8.19 (s, 1H), 8.10 (s, 1H), 7.51-7.44 (m, 2H), 6.75 (d, J = 8.4 Hz, 2H), 3.90 (s, 3H), 3.83 (s, 3H), 3.76 (s, 6H) | B |
| | 2,6-dimethoxy-N-{5-methoxy-6- | | |
| | [(1-methyl-1H-1,2,4-triazol-3- | | |
| | yl)amino]-1,2-benzoxazol-3- | | |
| | yl}benzene-1-sulfonamide | | |
| 36 | 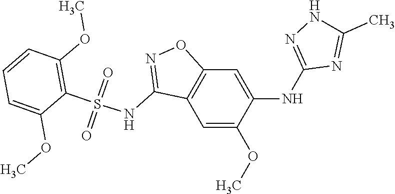 [SMILES: COc1cc2c(NS(=O)(=O)c3c(OC)cccc3OC)noc2cc1Nc1n[nH]c(C)n1] | MS (flow injection mode) m/z 460.9 [M + H]⁺; ¹H NMR (400 MHz, DMSO-d₆) δ 13.16 (br s, 1H), 11.14 (br s, 1H), 8.20 (br s, 1H), 7.91 (br s, 1H), 7.54-7.42 (m, 2H), 6.74 (d, J = 8.5 Hz, 2H), 3.90 (s, 3H), 3.76 (s, 6H), 2.32 (br s, 3H) | B |
| | 2,6-dimethoxy-N-{5-methoxy-6- | | |
| | [(5-methyl-1H-1,2,4-triazol-3- | | |
| | yl)amino]-1,2-benzoxazol-3- | | |
| | yl}benzene-1-sulfonamide | | |
| 37 | 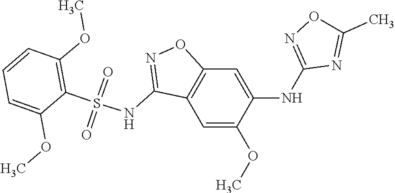 [SMILES: COc1cc2c(NS(=O)(=O)c3c(OC)cccc3OC)noc2cc1Nc1noc(C)n1] | MS (flow injection mode) m/z 483.9 [M + Na]⁺; ¹H NMR (400 MHz, DMSO-d₆) δ 11.18 (br s, 1H), 8.99 (s, 1H), 7.92 (s, 1H), 7.57 (s, 1H), 7.47 (t, J = 8.5 Hz, 1H), 6.74 (d, J = 8.5 Hz, 2H), 3.89 (s, 3H), 3.76 (s, 6H), 2.55 (s, 3H) | B |
| | 2,6-dimethoxy-N-{5-methoxy-6- | | |
| | [(5-methyl-1,2,4-oxadiazol-3- | | |
| | yl)amino]-1,2-benzoxazol-3- | | |
| | yl}benzene-1-sulfonamide | | |
| 38 | 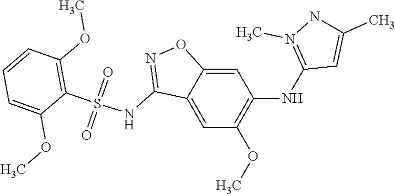 | LCMS m/z 474.1 [M + H]⁺; ¹H NMR (400 MHz, DMSO-d₆) δ 11.01 (s, 1H), 7.82 (s, 1H), 7.50- 7.44 (m, 2H), 6.74 (d, J = 8.8 Hz, 2H), 6.44 (s, 1H), 5.94 (s, 1H), 3.89 (s, 3H), 3.77 (s, 6H), 3.52 (s, 3H), 2.13 (s, 3H) | B |
| | N-{6-[(1,3-dimethyl-1H-pyrazol-5- | | |
| | yl)amino]-5-methoxy-1,2- | | |
| | benzoxazol-3-yl}-2,6- | | |
| | dimethoxybenzene-1-sulfonamide | | |
| 39 | 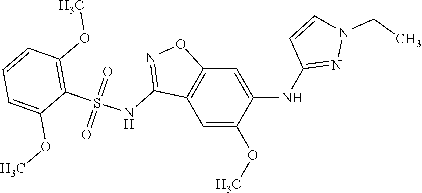 [SMILES: CCn1ccc(Nc2cc3onc(NS(=O)(=O)c4c(OC)cccc4OC)c3cc2OC)n1] | MS (flow injection mode) m/z 473.9 [M + H]⁺; ¹H NMR (400 MHz, DMSO-d₆) δ 10.98 (s, 1H), 8.25 (s, 1H), 8.15 (s, 1H), 7.58 (d, J = 2.3 Hz, 1H), 7.47 (t, J = 8.4 Hz, 1H), 7.42 (s, 1H), 6.74 (d, J = 8.5 Hz, 2H), 6.06 (d, J = 2.3 Hz, 1H), 4.07 (q, J = 7.3 Hz, 2H), 3.89 (s, 3H), 3.76 (s, 6H), 1.37 (t, J = 7.3 Hz, 3H) | B |
| | N-{6-[(1-ethyl-1H-pyrazol-3- | | |
| | yl)amino]-5-methoxy-1,2- | | |
| | benzoxazol-3-yl}-2,6- | | |
| | dimethoxybenzene-1-sulfonamide | | |
| 40 | 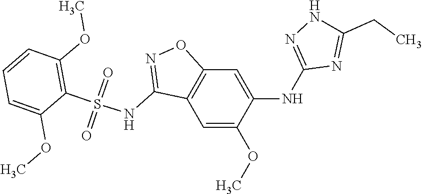 [SMILES: CCc1nc(Nc2cc3onc(NS(=O)(=O)c4c(OC)cccc4OC)c3cc2OC)n[nH]1] | LCMS m/z 475.0 [M + H]⁺; ¹H NMR (400 MHz, DMSO-d₆) δ 13.18 (s, 1H), 11.06 (s, 1H), 8.19 (s, 1H), 7.94 (s, 1H), 7.51- 7.43 (m, 2H), 6.74 (d, J = 8.5 Hz, 2H), 3.89 (s, 3H), 3.76 (s, 6H), 2.69 (q, J = 7.5 Hz, 2H), 1.24 (t, J = 7.7 Hz, 3H) | B |
| | N-{6-[(5-ethyl-1H-1,2,4-triazol-3- | | |
| | yl)amino]-5-methoxy-1,2- | | |
| | benzoxazol-3-yl}-2,6- | | |
| | dimethoxybenzene-1-sulfonamide | | |
| 41 | 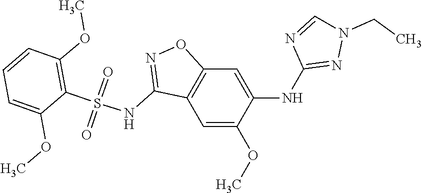 [SMILES: CCn1cnc(Nc2cc3onc(NS(=O)(=O)c4c(OC)cccc4OC)c3cc2OC)n1] | MS (flow injection mode) m/z 475.0 [M + H]⁺; ¹H NMR (400 MHz, DMSO-d₆) δ 11.07 (br s, 1H), 8.36 (s, 1H), 8.19 (s, 1H), 8.08 (s, 1H), 7.53-7.41 (m, 2H), 6.74 (d, J = 8.5 Hz, 2H), 4.15 (q, J = 7.3 Hz, 2H), 3.89 (s, 3H), 3.75 (s, 6H), 1.41 (t, J = 7.3 Hz, 3H) | B |
| | N-{6-[(1-ethyl-1H-1,2,4-triazol-3- | | |
| | yl)amino]-5-methoxy-1,2- | | |
| | benzoxazol-3-yl}-2,6- | | |
| | dimethoxybenzene-1-sulfonamide | | |
| 42 | 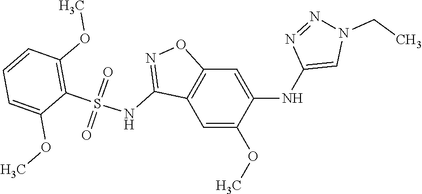 [SMILES: CCn1cc(Nc2cc3onc(NS(=O)(=O)c4c(OC)cccc4OC)c3cc2OC)nn1] | LCMS m/z 475.1 [M + H]⁺; ¹H NMR (400 MHz, DMSO-d₆) δ 11.00 (br s, 1H), 8.30 (s, 1H), 7.88 (s, 1H), 7.66 (s, 1H), 7.54- 7.39 (m, 2H), 6.74 (d, J = 8.5 Hz, 2H), 4.36 (q, J = 7.3 Hz, 2H), 3.88 (s, 3H), 3.75 (s, 6H), 1.44 (t, J = 7.4 Hz, 3H) | B |
| | N-{6-[(1-ethyl-1H-1,2,3-triazol-4- | | |
| | yl)amino]-5-methoxy-1,2- | | |
| | benzoxazol-3-yl}-2,6- | | |
| | dimethoxybenzene-1-sulfonamide | | |
| 43 | 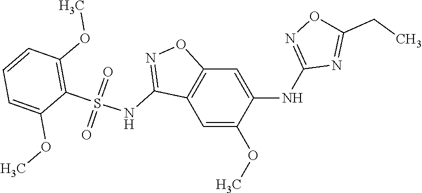 [SMILES: CCc1nc(Nc2cc3onc(NS(=O)(=O)c4c(OC)cccc4OC)c3cc2OC)no1] | MS (flow injection mode) m/z 497.9 [M + Na]⁺; ¹H NMR (400 MHz, DMSO-d₆) δ 11.17 (br s, 1H), 9.02 (s, 1H), 7.93 (s, 1H), 7.57 (s, 1H), 7.47 (t, J = 8.5 Hz, 1H), 6.75 (d, J = 8.6 Hz, 2H), 3.88 (s, 3H), 3.76 (s, 6H), 2.90 (q, J = 7.5 Hz, 2H), 1.30 (t, J = 7.5 Hz, 3H) | B |
| | N-{6-[(5-ethyl-1,2,4-oxadiazol-3- | | |
| | yl)amino]-5-methoxy-1,2- | | |
| | benzoxazol-3-yl}-2,6- | | |
| | dimethoxybenzene-1-sulfonamide | | |
| 44 | 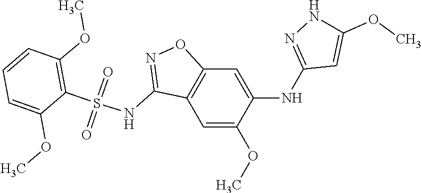 [SMILES: COc1cc(Nc2cc3onc(NS(=O)(=O)c4c(OC)cccc4OC)c3cc2OC)n[nH]1] | MS (flow injection mode) m/z 475.9 [M + H]⁺; ¹H NMR (400 MHz, DMSO-d₆) δ 11.76 (br s, 0.5H), 11.30 (br s, 0.5H), 11.0- 10.93 (m, 1H), 8.18 (s, 0.5H), 8.12 (s, 1H), 7.38-7.53 (m, 2H), 6.94 (br s, 0.5H), 6.73 (d, J = 8.6 Hz, 2H), 5.65 (br s, 0.5H), 5.51 (br s, 0.5H), 3.87 (s, 3H), 3.82- 3.71 (m, 9H) [mixture of tautomers] | B |
| | 2,6-dimethoxy-N-{5-methoxy-6- | | |
| | [(5-methoxy-1H-pyrazol-3- | | |
| | yl)amino]-1,2-benzoxazol-3- | | |
| | yl}benzene-1-sulfonamide | | |
| 45 | 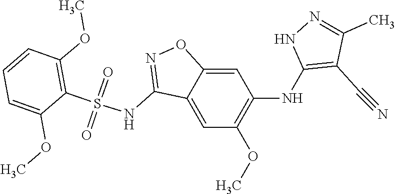 [SMILES: COc1cc2c(NS(=O)(=O)c3c(OC)cccc3OC)noc2cc1Nc1[nH]nc(C)c1C#N] | LCMS m/z 485.0 [M + H]⁺; ¹H NMR (400 MHz, DMSO-d₆) δ 13.01 (br s, 1H), 11.09 (br s, 1H), 7.85 (br s, 2H), 7.52 (s, 1H), 7.47 (t, J = 8.4 Hz, 1H), 6.74 (d, J = 8.4 Hz, 2H), 3.92 (s, 3H), 3.76 (s, 6H), 2.36 (s, 3H) | B |
| | N-{6-[(4-cyano-3-methyl-1H- | | |
| | pyrazol-5-yl)amino]-5-methoxy- | | |
| | 1,2-benzoxazol-3-yl}-2,6- | | |
| | dimethoxybenzene-1-sulfonamide | | |
| 46 | 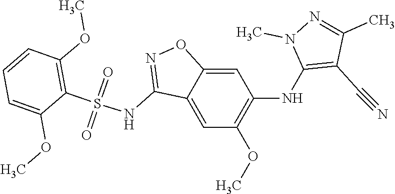 [SMILES: COc1cc2c(NS(=O)(=O)c3c(OC)cccc3OC)noc2cc1Nc1c(C#N)c(C)nn1C] | LCMS m/z 499.0 [M + H]⁺; ¹H NMR (400 MHz, DMSO-d₆) δ 11.11 (s, 1H), 8.46 (s, 1H), 7.56 (s, 1H), 7.47 (t, J = 8.5 Hz, 1H), 6.74 (d, J = 8.5 Hz, 2H), 6.64 (s, 1H), 3.91 (s, 3H), 3.76 (s, 6H), 3.60 (s, 3H), 2.24 (s, 3H) | B |
| | N-{6-[(4-cyano-1,3-dimethyl-1H- | | |
| | pyrazol-5-yl)amino]-5-methoxy- | | |
| | 1,2-benzoxazol-3-yl}-2,6- | | |
| | dimethoxybenzene-1-sulfonamide | | |
| 47 | 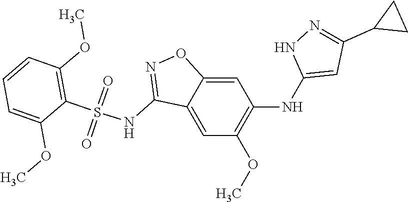 [SMILES: COc1cc2c(NS(=O)(=O)c3c(OC)cccc3OC)noc2cc1Nc1cc(C2CC2)n[nH]1] | LCMS m/z 486.0 [M + H]⁺; ¹H NMR (400 MHz, DMSO-d₆) δ 11.91 (br s, 1H), 10.97 (s, 1H), 8.12 (br s, 1H), 8.06 (s, 1H), 7.46 (t, J = 8.4 Hz, 1H), 7.40 (s, 1H), 6.74 (d, J = 8.5 Hz, 2H), 5.79 (s, 1H), 3.88 (s, 3H), 3.76 (s, 6H), 1.91-1.81 (m, 1H), 0.96- 0.87 (m, 2H), 0.70-0.62 (m, 2H) | B |
| | N-{6-[(3-cyclopropyl-1H-pyrazol-5- | | |
| | yl)amino]-5-methoxy-1,2- | | |
| | benzoxazol-3-yl}-2,6- | | |
| | dimethoxybenzene-1-sulfonamide | | |
| 48 | 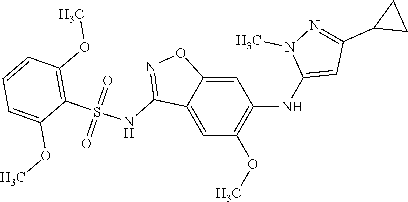 [SMILES: COc1cc2c(NS(=O)(=O)c3c(OC)cccc3OC)noc2cc1Nc1cc(C2CC2)nn1C] | LCMS m/z 500.1 [M + H]⁺; ¹H NMR (400 MHz, DMSO-d₆) δ 11.01 (br s, 1H), 7.79 (s, 1H), 7.51-7.43 (m, 2H), 6.74 (d, J = 8.6 Hz, 2H), 6.44 (s, 1H), 5.88 (s, 1H), 3.88 (s, 3H), 3.76 (s, 6H), 3.51 (s, 3H), 1.86-1.75 (m, 1H), 0.86-0.77 (m, 2H), 0.68- 0.60 (m, 2H) | B |
| | N-{6-[(3-cyclopropyl-1-methyl-1H- | | |
| | pyrazol-5-yl)amino]-5-methoxy- | | |
| | 1,2-benzoxazol-3-yl}-2,6- | | |
| | dimethoxybenzene-1-sulfonamide | | |
| 49 | 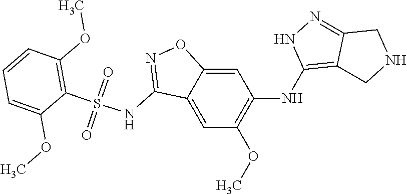 [SMILES: COc1cc2c(NS(=O)(=O)c3c(OC)cccc3OC)noc2cc1Nc1[nH]nc2c1CNC2] | LCMS m/z 487.0 [M + H]⁺; ¹H NMR (400 MHz, DMSO-d₆) δ 12.03 (br s, 1H), 7.95 (s, 1H), 7.41 (t, J = 8.4 Hz, 1H), 7.35 (s, 1H), 6.71 (d, J = 8.5 Hz, 1H), 3.98 (s, 2H), 3.90 (s, 2H), 3.86 (s, 3H), 3.72 (s, 6H) | B |
| | 2,6-dimethoxy-N-{5-methoxy-6- | | |
| | [(2,4,5,6-tetrahydropyrrolo[3,4- | | |
| | c]pyrazol-3-yl)amino]-1,2- | | |
| | benzoxazol-3-yl}benzene-1- | | |
| | sulfonamide | | |
| 50 | 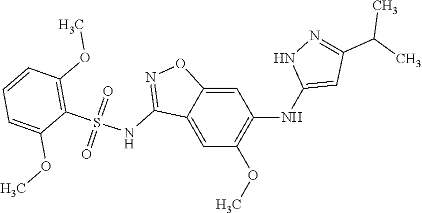 [SMILES: COc1cc2c(NS(=O)(=O)c3c(OC)cccc3OC)noc2cc1Nc1cc(C(C)C)n[nH]1] | LCMS m/z 488.1 [M + H]⁺; ¹H NMR (400 MHz, DMSO-d₆) δ 11.87 (br s, 1H), 8.10 (br s, 1H), 7.97 (s, 1H), 7.39 (t, J = 8.4 Hz, 1H), 7.30 (s, 1H), 6.70 (d, J = 8.4 Hz, 2H), 5.87 (s, 1H), 3.86 (s, 3H), 3.71 (s, 6H), 2.90 (td, J = 6.8, 13.7 Hz, 1H), 1.21 (d, J = 6.8 Hz, 6H) | B |
| | 2,6-dimethoxy-N-(5-methoxy-6- | | |
| | {[3-(propan-2-yl)-1H-pyrazol-5- | | |
| | yl]amino}-1,2-benzoxazol-3- | | |
| | yl)benzene-1-sulfonamide | | |
| 51 | 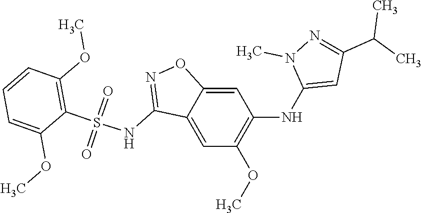 [SMILES: COc1cc2c(NS(=O)(=O)c3c(OC)cccc3OC)noc2cc1Nc1cc(C(C)C)nn1C] | LCMS m/z 502.2 [M + H]⁺; ¹H NMR (400 MHz, DMSO-d₆) δ 10.99 (br s, 1H), 7.79 (s, 1H), 7.52-7.41 (m, 2H), 6.72 (d, J = 8.5 Hz, 2H), 6.43 (s, 1H), 5.99 (s, 1H), 3.88 (s, 3H), 3.76 (s, 6H), 3.54 (s, 3H), 2.82 (sept, J = 6.9 Hz, 1H), 1.19 (d, J = 6.9 Hz, 6H) | B |
| | 2,6-dimethoxy-N-(5-methoxy-6- | | |
| | {[1-methyl-3-(propan-2-yl)-1H- | | |
| | pyrazol-5-yl]amino}-1,2- | | |
| | benzoxazol-3-yl)benzene-1- | | |
| | sulfonamide | | |
| 52 | 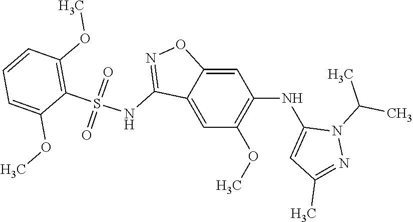 [SMILES: COc1cc2c(NS(=O)(=O)c3c(OC)cccc3OC)noc2cc1Nc1cc(C)nn1C(C)C] | LCMS m/z 502.2 [M + H]⁺; ¹H NMR (400 MHz, DMSO-d₆) δ 11.00 (br s, 1H), 7.78 (s, 1H), 7.50-7.43 (m, 2H), 6.74 (d, J = 8.5 Hz, 2H), 6.29 (s, 1H), 5.89 (s, 1H), 4.32 (sept, J = 6.6 Hz, 1H), 3.89 (s, 3H), 3.77 (s, 6H), 2.17 (s, 3H), 1.26 (d, J = 6.8 Hz, 6H) | B |
| | 2,6-dimethoxy-N-(5-methoxy-6- | | |
| | {[3-methyl-1-(propan-2-yl)-1H- | | |
| | pyrazol-5-yl]amino}-1,2- | | |
| | benzoxazol-3-yl)benzene-1- | | |
| | sulfonamide | | |
| 53 | 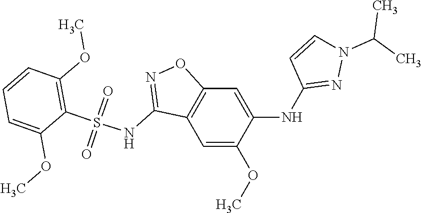 [SMILES: COc1cc2c(NS(=O)(=O)c3c(OC)cccc3OC)noc2cc1Nc1ccn(C(C)C)n1] | LCMS m/z 487.9 [M + H]⁺; ¹H NMR (400 MHz, DMSO-d₆) δ 10.98 (br s, 1H), 8.23 (s, 1H), 8.14 (s, 1H), 7.60 (d, J = 2.5 Hz, 1H), 7.46 (t, J = 8.5 Hz, 1H), 7.43 (s, 1H), 6.74 (d, J = 8.5 Hz, 2H), 6.06 (d, J = 2.3 Hz, 1H), 4.41 (sept, J = 6.7 Hz, 1H), 3.89 (s, 3H), 3.76 (s, 6H), 1.42 (d, J = 6.5 Hz, 6H) | B |
| | 2,6-dimethoxy-N-(5-methoxy-6- | | |
| | {[1-(propan-2-yl)-1H-pyrazol-3- | | |
| | yl]amino}-1,2-benzoxazol-3- | | |
| | yl)benzene-1-sulfonamide | | |
| 54 | 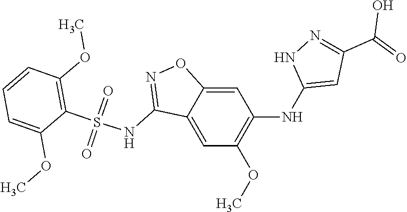 [SMILES: COc1cc2c(NS(=O)(=O)c3c(OC)cccc3OC)noc2cc1Nc1cc(C(=O)O)n[nH]1] | LCMS m/z 490.0 [M + H]⁺; ¹H NMR (400 MHz, DMSO-d₆) δ 13.19 (br s, 2H), 11.02 (br s, 1H), 8.48 (s, 1H), 8.16 (br s, 1H), 7.50-7.44 (m, 2H), 6.74 (d, J = 8.6 Hz, 2H), 6.59 (s, 1H), 3.90 (s, 3H), 3.77 (s, 6H) | Ester Hydrolysis of Ex 55 |
| | 5-({3-[(2,6-dimethoxybenzene-1- | | |
| | sulfonyl)amino]-5-methoxy-1,2- | | |
| | benzoxazol-6-yl}amino)-1H- | | |
| | pyrazole-3-carboxylic acid | | |
| 55 | 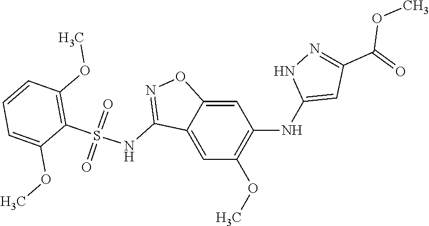 [SMILES: COC(=O)c1cc(Nc2cc3onc(NS(=O)(=O)c4c(OC)cccc4OC)c3cc2OC)[nH]n1] | LCMS m/z 504.0 [M + H]⁺; ¹H NMR (400 MHz, DMSO-d₆) δ 13.38 (br s, 1H), 11.03 (br s, 1H), 8.54 (s, 1H), 8.18 (s, 1H), 7.51-7.42 (m, 2H), 6.74 (d, J = 8.5 Hz, 2H), 6.66 (s, 1H), 3.91 (s, 3H), 3.84 (s, 3H), 3.76 (s, 6H) | B |
| | methyl 5-({3-[(2,6- | | |
| | dimethoxybenzene-1- | | |
| | sulfonyl)amino]-5-methoxy-1,2- | | |
| | benzoxazol-6-yl}amino)-1H- | | |
| | pyrazole-3-carboxylate | | |
| 56 | 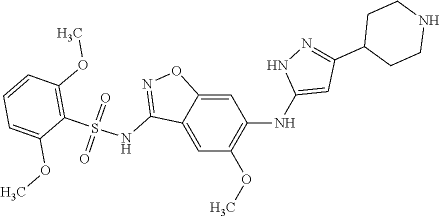 [SMILES: COc1cc2c(NS(=O)(=O)c3c(OC)cccc3OC)noc2cc1Nc1cc(C2CCNCC2)n[nH]1] | LCMS m/z 529.1 [M + H]⁺; ¹H NMR (400 MHz, DMSO-d₆) δ 11.93 (br s, 1H), 8.05 (br s, 1H), 7.91 (s, 1H), 7.34 (br t, J = 8.3 Hz, 1H), 7.24 (s, 1H), 6.67 (d, J = 8.4 Hz, 2H), 5.89 (s, 1H), 3.83 (s, 3H), 3.66 (s, 6H), 3.15 (br d, J = 12.1 Hz, 2H), 2.83-2.71 (m, 3H), 1.93 (br d, J = 12.2 Hz, 2H), 1.61 (br d, J = 12.7 Hz, 2H) | B |
| | 2,6-dimethoxy-N-(5-methoxy-6- | | |
| | {[3-(piperidin-4-yl)-1H-pyrazol-5- | | |
| | yl]amino}-1,2-benzoxazol-3- | | |
| | yl)benzene-1-sulfonamide | | |
| 57 | 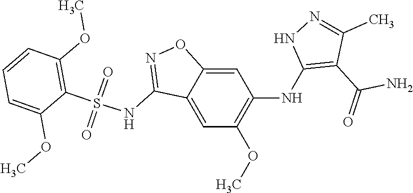 [SMILES: COc1cc2c(NS(=O)(=O)c3c(OC)cccc3OC)noc2cc1Nc1[nH]nc(C)c1C(N)=O] | LCMS m/z 503.0 [M + H]⁺; ¹H NMR (400 MHz, DMSO-d₆) δ 12.44 (br s, 1H), 11.07 (br s, 1H), 9.94 (br s, 1H), 8.33 (br s, 1H), 7.50 (br s, 2H), 6.99 (br s, 2H), 6.78 (br d, J = 8.3 Hz, 2H), 3.94 (br s, 3H), 3.80 (br s, 6H), 2.49 (br s, 3H) | B |
| | 5-({3-[(2,6-dimethoxybenzene-1- | | |
| | sulfonyl)amino]-5-methoxy-1,2- | | |
| | benzoxazol-6-yl}amino)-3-methyl- | | |
| | 1H-pyrazole-4-carboxamide | | |
| 58 | 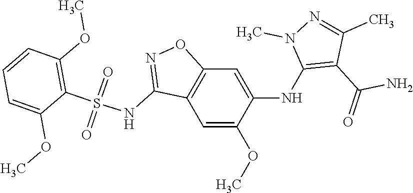 [SMILES: COc1cc2c(NS(=O)(=O)c3c(OC)cccc3OC)noc2cc1Nc1c(C(N)=O)c(C)nn1C] | LCMS m/z 517.2 [M + H]⁺; ¹H NMR (400 MHz, DMSO-d₆) δ 11.20 (s, 1H), 8.51 (s, 1H), 8.49 (s, 1H), 7.63 (s, 1H), 7.48 (t, J = 8.5 Hz, 1H), 6.75 (d, J = 8.5 Hz, 2H), 6.39 (s, 2H), 3.94 (s, 3H), 3.76 (s, 6H), 3.50 (s, 3H), 2.40 (s, 3H) | B |
| | 5-({3-[(2,6-dimethoxybenzene-1- | | |
| | sulfonyl)amino]-5-methoxy-1,2- | | |
| | benzoxazol-6-yl}amino)-1,3- | | |
| | dimethyl-1H-pyrazole-4- | | |
| | carboxamide | | |
| 59 | 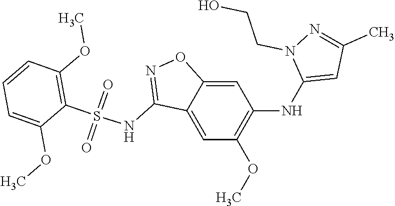 [SMILES: COc1cc2c(NS(=O)(=O)c3c(OC)cccc3OC)noc2cc1Nc1cc(C)nn1CCO] | LCMS m/z 504.1 [M + H]⁺; ¹H NMR (400 MHz, DMSO-d₆) δ 11.09 (br s, 1H), 8.29 (s, 1H), 7.58-7.46 (m, 2H), 6.86 (s, 1H), 6.80 (d, J = 8.4 Hz, 2H), 6.09 (s, 1H), 5.47 (br s, 1H), 4.06 (br t, J = 4.6 Hz, 2H), 3.94 (s, 3H), 3.82 (s, 6H), 3.75 (br s, 2H), 2.21 (s, 3H) | B |
| | N-(6-{[1-(2-hydroxyethyl)-3- | | |
| | methyl-1H-pyrazol-5-yl]amino}-5- | | |
| | methoxy-1,2-benzoxazol-3-yl)-2,6- | | |
| | dimethoxybenzene-1-sulfonamide | | |
| 60 | 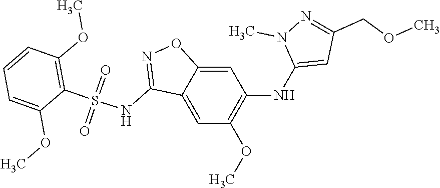 [SMILES: COCc1cc(Nc2cc3onc(NS(=O)(=O)c4c(OC)cccc4OC)c3cc2OC)n(C)n1] | LCMS m/z 504.1 [M + H]⁺; ¹H NMR (400 MHz, DMSO-d₆) δ 11.03 (s, 1H), 7.90 (s, 1H), 7.52- 7.43 (m, 2H), 6.74 (d, J = 8.6 Hz, 2H), 6.43 (s, 1H), 6.14 (s, 1H), 4.29 (s, 2H), 3.89 (s, 3H), 3.77 (s, 6H), 3.58 (s, 3H), 3.26 (s, 3H) | B |
| | 2,6-dimethoxy-N-(5-methoxy-6- | | |
| | {[3-(methoxymethyl)-1-methyl-1H- | | |
| | pyrazol-5-yl]amino}-1,2- | | |
| | benzoxazol-3-yl)benzene-1- | | |
| | sulfonamide | | |
| 61 | 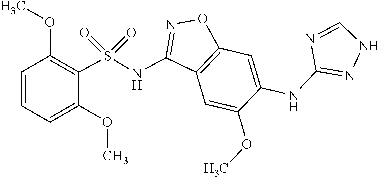 [SMILES: COc1cc2c(NS(=O)(=O)c3c(OC)cccc3OC)noc2cc1Nc1nc[nH]n1] | LCMS m/z 447.0 [M + H]⁺; ¹H NMR (400 MHz, DMSO-d₆) δ 11.08 (br s, 1H), 8.81-7.82 (m, 3H), 7.54-7.41 (m, 2H), 6.74 (d, J = 8.5 Hz, 2H), 3.91 (s, 3H), 3.75 (s, 6H) | |
| | 2,6-dimethoxy-N-{5-methoxy-6- | | |
| | [(1H-1,2,4-triazol-3-yl)amino]-1,2- | | |
| | benzoxazol-3-yl}benzene-1- | | |
| | sulfonamide | | |
| 62 | 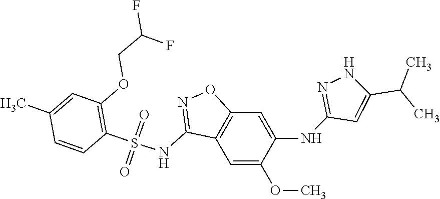 [SMILES: COc1cc2c(NS(=O)(=O)c3ccc(C)cc3OCC(F)F)noc2cc1Nc1cc(C(C)C)[nH]n1] | LCMS m/z 521.9 [M + H]⁺; ¹H NMR (400 MHz, DMSO-d₆) δ 11.90 (br s, 1H), 11.09 (br s, 1H), 8.16 (s, 1H), 8.11 (s, 1H), 7.86-7.65 (m, 1H), 7.38 (s, 1H), 6.99 (s, 1H), 6.88 (d, J = 8.0 Hz, 1H), 5.88 (s, 1H), 4.22-3.99 (m, 2H), 3.93-3.83 (m, 3H), 2.94- 2.87 (m, 1H), 2.33 (s, 3H) 1.26- 1.17 (m, 9H) | B |
| | 2-(2,2-difluoroethoxy)-N-(5- | | |
| | methoxy-6-{[5-(propan-2-yl)-1H- | | |
| | pyrazol-3-yl]amino}-1,2- | | |
| | benzoxazol-3-yl)-4- | | |
| | methylbenzene-1-sulfonamide | | |
| 63 | 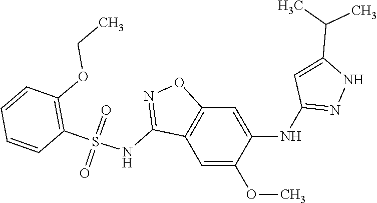 [SMILES: CCOc1ccccc1S(=O)(=O)Nc1noc2cc(Nc3cc(C(C)C)[nH]n3)c(OC)cc12] | LCMS m/z 472.4 [M + H]⁺; ¹H NMR (400 MHz, DMSO-d₆) δ 11.90 (br s, 1H), 11.21 (br s, 1H), 8.29-8.00 (m, 2H), 7.87 (dd, J = 1.5, 7.8 Hz, 1H), 7.56 (br t, J = 7.8 Hz, 1H), 7.35 (s, 1H), 7.16 (d, J = 8.5 Hz, 1H), 7.07 (t, J = 7.5 Hz, 1H), 5.87 (s, 1H), 4.12 (q, J = 6.9 Hz, 2H), 3.88 (s, 3H), 2.93-1.86 (m, 1H), 1.32-1.11 (m, 9H) | B |
| | 2-ethoxy-N-(5-methoxy-6-{[5- | | |
| | (propan-2-yl)-1H-pyrazol-3- | | |
| | yl]amino}-1,2-benzoxazol-3- | | |
| | yl)benzene-1-sulfonamide | | |
| 64 | 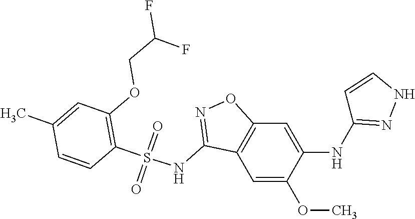 [SMILES: COc1cc2c(NS(=O)(=O)c3ccc(C)cc3OCC(F)F)noc2cc1Nc1cc[nH]n1] | LCMS m/z 480 [M + H]⁺; ¹H NMR (400 MHz, DMSO-d₆) δ 8.25 (s, 1H), 8.15 (br d, J = 7.5 Hz, 1H), 7.77 (d, J = 8.0 Hz, 1H), 7.59 (d, J = 1.9 Hz, 1H), 7.33 (s, 1H), 7.13 (s, 1H), 6.98 (d, J = 8.0 Hz, 1H), 6.60-6.26 (m, 1H), 6.11 (d, J = 1.9 Hz, 1H), 4.42 (dt, J = 3.8, 13.9 Hz, 2H), 3.90 (s, 3H), 2.34 (s, 3H) | B |
| | 2-(2,2-difluoroethoxy)-N-{5- | | |
| | methoxy-6-[(1H-pyrazol-3- | | |
| | yl)amino]-1,2-benzoxazol-3-yl}-4- | | |
| | methylbenzene-1-sulfonamide | | |
| 65 | 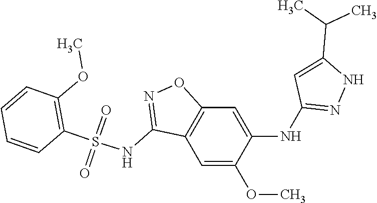 [SMILES: COc1cc2c(NS(=O)(=O)c3ccccc3OC)noc2cc1Nc1cc(C(C)C)[nH]n1] | LCMS m/z 457.9 [M + H]⁺; ¹H NMR (500 MHz, DMSO-d₆) δ 11.91 (br s, 1H), 11.29 (br s, 1H), 8.21-8.09 (m, 2H), 7.86 (dd, J = 1.5, 7.9 Hz, 1H), 7.61 (t, J = 7.9 Hz, 1H), 7.35 (s, 1H), 7.18 (d, J = 8.2 Hz, 1H), 7.10 (t, J = 7.6 Hz, 1H), 5.88 (s, 1H), 3.90 (s, 3H), 3.83 (s, 3H), 2.92-2.85 (m, 1H), 1.21 (d, J = 6.9 Hz, 6H) | B |
| | 2-methoxy-N-(5-methoxy-6-{[5- | | |
| | (propan-2-yl)-1H-pyrazol-3- | | |
| | yl]amino}-1,2-benzoxazol-3- | | |
| | yl)benzene-1-sulfonamide | | |
| 66 | 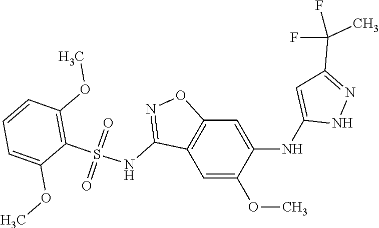 [SMILES: COc1cc2c(NS(=O)(=O)c3c(OC)cccc3OC)noc2cc1Nc1cc(C(C)(F)F)n[nH]1] | LCMS m/z 510 [M + H]⁺; ¹H NMR (400 MHz, DMSO-d6) · 13.10-12.74 (m, 1 H), 10.96 (s, 1H), 8.33 (br s, 1H), 7.96-8.22 (m, 1H), 7.50-7.42 (m, 2H), 6.74 (d, J = 8.5 Hz, 2H), 6.37 (s, 1H), 3.89 (s, 3H), 3.76 (s, 6H), 2.00 (t, J = 18.8 Hz, 3H) | B |
| | N-(6-{[3-(1,1-difluoroethyl)-1H- | | |
| | pyrazol-5-yl]amino}-5-methoxy- | | |
| | 1,2-benzoxazol-3-yl)-2,6- | | |
| | dimethoxybenzene-1-sulfonamide | | |
| 67 | 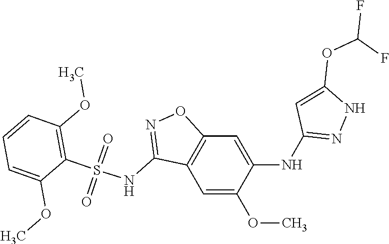 [SMILES: COc1cc2c(NS(=O)(=O)c3c(OC)cccc3OC)noc2cc1Nc1cc(OC(F)F)[nH]n1] | LCMS m/z 511 [M + H]⁺; ¹H NMR (600 MHz, DMSO-d₆) δ 11.92 (s, 1H), 11.02 (br s, 1H), 8.39-8.17 (m, 1H), 7.50 (s, 1H) 7.46 (t, J = 8.4 Hz, 1H), 7.26 (t, J = 73.8 Hz, 1H), 6.96 (s, 1H), 6.74 (d, J = 8.4 Hz, 2H), 5.94 (d, J = 1.4 Hz, 1H), 3.89 (s, 3H), 3.76 (s, 6H) | B |
| | N-(6-{[5-(difluoromethoxy)-1H- | | |
| | pyrazol-3-yl]amino}-5-methoxy- | | |
| | 1,2-benzoxazol-3-yl)-2,6- | | |
| | dimethoxybenzene-1-sulfonamide | | |
| 68 | 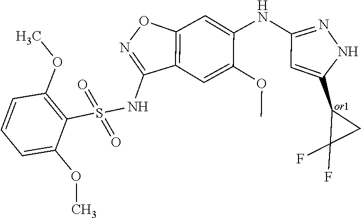 [SMILES: COc1cc2c(NS(=O)(=O)c3c(OC)cccc3OC)noc2cc1Nc1cc([C@H]2CC2(F)F)[nH]n1] N-(6-{[5-(2,2-difluorocyclopropyl)- 1H-pyrazol-3-yl]amino}-5- | SFC MS m/z 522 [M + H]⁺; [α]_{D}²² = +18.3° (C 0.1, MeOH); ¹H NMR (600 MHz, DMSO-d₆) δ 12.21 (br s, 1H), 10.94 (s, 1H), 8.18 (s, 1H), 8.12 (s, 1H), 7.46 (t, J = 8.4 Hz, 1H), 7.41 (s, 1H), 6.74 (d, J = 8.5 Hz, 2H), 6.01 (br s, 1H), 3.88 (s, 3H), 3.76 (s, 6H), 3.03-2.87 (m, 1H), 2.14- 1.99 (m, 1H), 1.89-1.76 (m, 1H). Column: Chiralpak ® AD SFC (20 × 250 mm; 10 micron); Mobile phase A: CO₂; Mobile phase B: Isopropanol + 10 mM NH₃; 50% B isocratic, 100 bar, 80 mL/min; Rₜ = 3.96 min | |
| | methoxy-1,2-benzoxazol-3-yl)-2,6- | | |
| | dimethoxybenzene-1-sulfonamide | | |
| | or1 = single enantiomer absolute | | |
| | stereochemistry unknown | | |
| 69 | 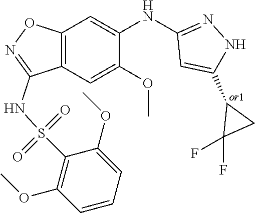 [SMILES: COc1cc2c(NS(=O)(=O)c3c(OC)cccc3OC)noc2cc1Nc1cc([C@@H]2CC2(F)F)[nH]n1] N-(6-{[5-(2,2-difluorocyclopropyl)- 1H-pyrazol-3-yl]amino}-5- | SFC MS m/z 522 [M + H]⁺; [α]_{D}²² = −10.1° (C 0.1, MeOH); ¹H NMR (600 MHz, DMSO-d₆) δ 12.21 (br s, 1H), 10.94 (s, 1H), 8.18 (s, 1H), 8.12 (s, 1H), 7.46 (t, J = 8.4 Hz, 1H), 7.41 (s, 1H), 6.74 (d, J = 8.5 Hz, 2H), 6.01 (s, 1H), 3.88 (s, 3H), 3.76 (s, 6H), 3.01-2.88 (m, 1H), 2.12-2.01 (m, 1H), 1.90-1.78 (m, 1H). Column: Chiralpak ® AD SFC (20 × 250 mm; 10 micron); Mobile phase A: CO₂; Mobile phase B: Isopropanol + 10 mM NH₃; 50% B isocratic, 100 bar, 80 mL/min; Rₜ = 7.97 min | B |
| | methoxy-1,2-benzoxazol-3-yl)-2,6- | | |
| | dimethoxybenzene-1-sulfonamide | | |
| | or1 = single enantiomer absolute | | |
| | stereochemistry unknown | | |
| 70 | 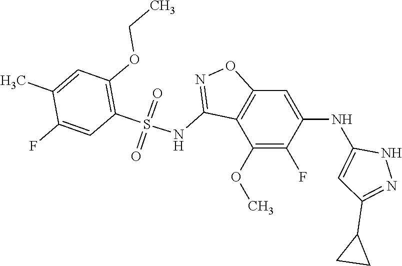 [SMILES: CCOc1cc(C)c(F)cc1S(=O)(=O)Nc1noc2cc(Nc3cc(C4CC4)n[nH]3)c(F)c(OC)c12] | LCMS m/z 520 [M + H]⁺; ¹H NMR (400 MHz, METHANOL-d₄) δ 7.61 (d, J = 8.9 Hz, 1H), 7.42 (br s, 1H), 7.02 (d, J = 5.8 Hz, 1H), 5.72 (s, 1H), 4.20-4.08 (m, 5H), 2.30 (d, J = 1.8 Hz, 3H), 1.93-1.85 (m, 1H), 1.36 (t, J = 7.0 Hz, 3H), 1.01-0.94 (m, 2H), 0.76-0.69 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-2-ethoxy-5- | | |
| | fluoro-4-methylbenzene-1- | | |
| | sulfonamide | | |
| 71 | 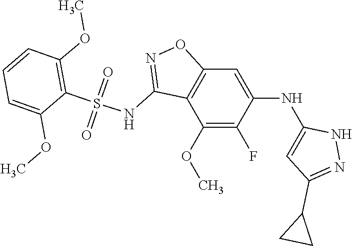 [SMILES: COc1cccc(OC)c1S(=O)(=O)Nc1noc2cc(Nc3cc(C4CC4)n[nH]3)c(F)c(OC)c12] | LCMS m/z 504 [M + H]⁺; ¹H NMR (400 MHz, DMSO-d₆) δ 11.98 (br s, 1H), 9.74 (br s, 1H), 8.73 (br s, 1H), 7.90 (br d, J = 4.7 Hz, 1H), 7.48 (br t, J = 8.5 Hz, 1H), 6.77 (d, J = 8.4 Hz, 2H), 5.73 (s, 1H), 4.05 (d, J = 2.6 Hz, 3H), 3.77 (s, 6H), 1.92-1.82 (m, 1H), 0.95-0.87 (m, 2H), 0.71-0.64 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-2,6- | | |
| | dimethoxybenzene-1-sulfonamide | | |
| 72 | 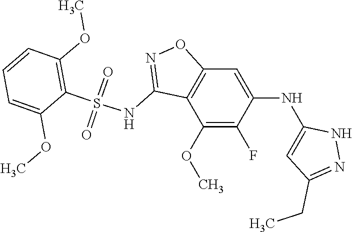 [SMILES: CCc1cc(Nc2cc3onc(NS(=O)(=O)c4c(OC)cccc4OC)c3c(OC)c2F)[nH]n1] | LCMS m/z 492 [M + H]⁺; ¹H NMR (400 MHz, DMSO-d₆) δ 11.97 (br s, 1H), 8.77 (br s, 1H), 7.94 (br s, 1H), 7.49 (br d, J = 4.7 Hz, 1H), 6.78 (br dd, J = 8.3, 4.5 Hz, 2H), 5.84 (br s, 1H), 4.05 (br s, 3H), 3.78 (br d, J = 4.3 Hz, 6H), 2.61-2.55 (m, 2H), 1.24-1.14 (m, 3H) | B |
| | N-{6-[(3-ethyl-1H-pyrazol-5- | | |
| | yl)amino]-5-fluoro-4-methoxy-1,2- | | |
| | benzoxazol-3-yl}-2,6- | | |
| | dimethoxybenzene-1-sulfonamide | | |
| 73 | 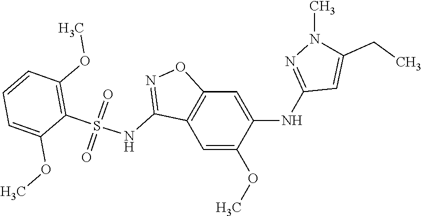 [SMILES: CCc1cc(Nc2cc3onc(NS(=O)(=O)c4c(OC)cccc4OC)c3cc2OC)nn1C] | LCMS m/z 488 [M + H]⁺; ¹H NMR (400 MHz, DMSO-d₆) δ 10.96 (br s, 1H), 8.18-8.10 (m, 2H), 7.46 (t, J = 8.4 Hz, 1H), 7.39 (s, 1H), 6.74 (d, J = 8.4 Hz, 2H), 5.89 (s, 1 H), 3.87 (s, 3H), 3.75 (s, 6H), 3.66 (s, 3H), 2.60-2.55 (m, 2H), 1.17 (t, J = 7.5 Hz, 3H) | B |
| | N-{6-[(5-ethyl-1-methyl-1H- | | |
| | pyrazol-3-yl)amino]-5-methoxy- | | |
| | 1,2-benzoxazol-3-yl}-2,6- | | |
| | dimethoxybenzene-1-sulfonamide | | |
| 74 | 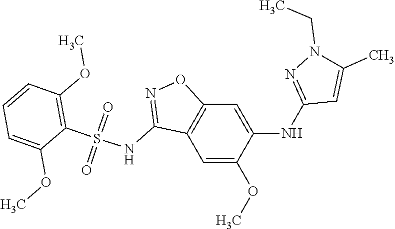 [SMILES: CCn1nc(Nc2cc3onc(NS(=O)(=O)c4c(OC)cccc4OC)c3cc2OC)cc1C] | LCMS m/z 488 [M + H]⁺; ¹H NMR (400 MHz, DMSO-d₆) δ 10.95 (br s, 1H), 8.15-8.09 (m, 2H), 7.45 (t, J = 8.4 Hz, 1H), 7.40 (s, 1H), 6.73 (d, J = 8.5 Hz, 2H), 5.86 (s, 1H), 3.97 (d, J = 7.1 Hz, 2H), 3.87 (s, 3H), 3.75 (s, 6H), 2.21 (s, 3H), 1.29 (t, J = 7.1 Hz, 3H) | B |
| | N-{6-[(1-ethyl-5-methyl-1H- | | |
| | pyrazol-3-yl)amino]-5-methoxy- | | |
| | 1,2-benzoxazol-3-yl}-2,6- | | |
| | dimethoxybenzene-1-sulfonamide | | |
| 75 | 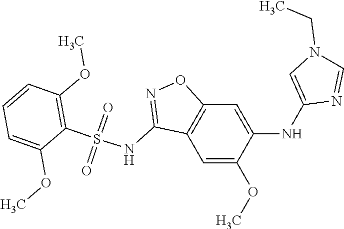 [SMILES: CCn1cnc(Nc2cc3onc(NS(=O)(=O)c4c(OC)cccc4OC)c3cc2OC)c1] | LCMS m/z 474 [M + H]⁺; ¹H NMR (400 MHz, CHLOROFORM-d) δ 8.11 (br s, 1H), 7.44 (s, 1H), 7.39 (t, J = 8.4 Hz, 1H), 7.34 (s, 1H), 7.03 (s, 1H), 6.96 (s, 1H), 6.76 (s, 1H), 6.60 (d, J = 8.5 Hz, 2H), 4.03- 3.98 (m, 2H), 3.97 (s, 3H), 3.92 (s, 6H), 1.50 (t, J = 7.3 Hz, 3H) | B |
| | N-{6-[(1-ethyl-1H-imidazol-4- | | |
| | yl)amino]-5-methoxy-1,2- | | |
| | benzoxazol-3-yl}-2,6- | | |
| | dimethoxybenzene-1-sulfonamide | | |
| 76 | 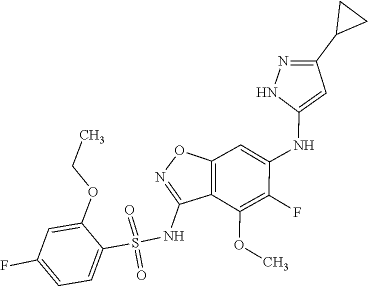 [SMILES: CCOc1cc(F)ccc1S(=O)(=O)Nc1noc2cc(Nc3cc(C4CC4)n[nH]3)c(F)c(OC)c12] | LCMS m/z 506 [M + H]⁺; ¹H NMR (400 MHz, METHANOL-d₄) δ 8.00 (dd, J = 8.9, 6.4 Hz, 1H), 7.41 (br s, 1H), 6.95 (dd, J = 10.9, 2.2 Hz, 1H), 6.83 (td, J = 8.4, 2.2 Hz, 1H), 5.72 (s, 1H), 4.20-4.11 (m, 5H), 1.93-1.84 (m, 1H), 1.38 (t, J = 7.0 Hz, 3H), 1.01-0.94 (m, 2H), 0.76-0.69 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-2-ethoxy-4- | | |
| | fluorobenzene-1-sulfonamide | | |
| 77 | 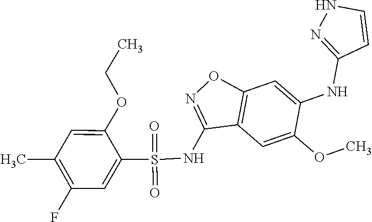 [SMILES: CCOc1cc(C)c(F)cc1S(=O)(=O)Nc1noc2cc(Nc3cc[nH]n3)c(OC)cc12] | LCMS m/z 462 [M + H]⁺; ¹H NMR (400 MHz, DMSO-d₆) δ 12.18 (br s, 1H), 11.33 (br s, 1H), 8.26 (s, 1H), 8.19 (s, 1H), 7.59 (s, 1H), 7.55 (d, J = 9.0 Hz, 1H), 7.38 (s, 1H), 7.13 (d, J = 5.9 Hz, 1H), 6.11 (s, 1H), 4.09 (q, J = 6.9 Hz, 2H), 3.90 (s, 3H), 2.26 (s, 3H), 1.21 (t, J = 6.9 Hz, 3 H) | B |
| | 2-ethoxy-5-fluoro-N-{5-methoxy-6- | | |
| | [(1H-pyrazol-3-yl)amino]-1,2- | | |
| | benzoxazol-3-yl}-4- | | |
| | methylbenzene-1-sulfonamide | | |
| 78 | 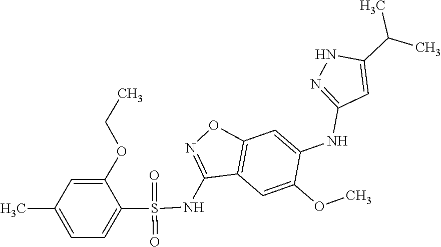 [SMILES: CCOc1cc(C)ccc1S(=O)(=O)Nc1noc2cc(Nc3cc(C(C)C)[nH]n3)c(OC)cc12] | LCMS m/z 486 [M + H]⁺; ¹H NMR (400 MHz, DMSO-d₆) δ 11.89 (br s, 1H), 11.08 (br s, 1H), 8.15 (s, 1H), 8.10 (s, 1H), 7.74 (d, J = 8.1 Hz, 1H), 7.38 (s, 1H), 6.98 (s, 1H), 6.87 (br d, J = 8.1 Hz, 1H), 5.87 (s, 1H), 4.15-4.04 (m, 2H), 3.88 (s, 3H) 2.94-2.84 (m, 1H), 2.32 (s, 3H), 1.26-1.17 (m, 9H) | B |
| | 2-ethoxy-N-(5-methoxy-6-{[5- | | |
| | (propan-2-yl)-1H-pyrazol-3- | | |
| | yl]amino}-1,2-benzoxazol-3-yl)-4- | | |
| | methylbenzene-1-sulfonamide | | |
| 79 | 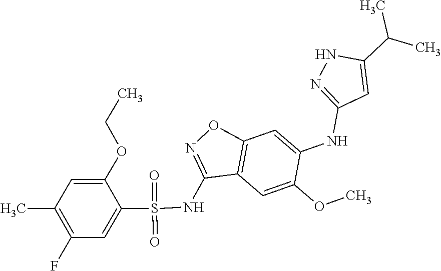 [SMILES: CCOc1cc(C)c(F)cc1S(=O)(=O)Nc1noc2cc(Nc3cc(C(C)C)[nH]n3)c(OC)cc12] | LCMS m/z 504 [M + H]⁺; ¹H NMR (400 MHz, DMSO-d₆) δ 11.90 (br s, 1H), 11.33 (br s, 1H), 8.16 (br s, 1H), 8.12 (br s, 1H), 7.55 (d, J = 9.0 Hz, 1H), 7.36 (s, 1H), 7.13 (br d, J = 5.8 Hz, 1H), 5.87 (s, 1H), 4.08 (q, J = 7.0 Hz, 2H), 3.88 (s, 3 H), 2.93-2.86 (m, 1H), 2.26 (d, J = 1.3 Hz, 3H), 1.23-1.18 (m, 9 H) | B |
| | 2-ethoxy-5-fluoro-N-(5-methoxy-6- | | |
| | {[5-(propan-2-yl)-1H-pyrazol-3- | | |
| | yl]amino}-1,2-benzoxazol-3-yl)-4- | | |
| | methylbenzene-1-sulfonamide | | |
| 80 | 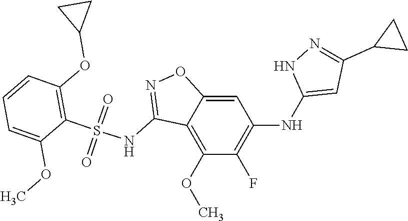 [SMILES: COc1cccc(OC2CC2)c1S(=O)(=O)Nc1noc2cc(Nc3cc(C4CC4)n[nH]3)c(F)c(OC)c12] | LCMS m/z 530 [M + H]⁺; ¹H NMR (400 MHz, CHLOROFORM-d) δ 7.46-7.34 (m, 2H), 7.01-6.92 (m, 1H), 6.65-6.57 (m, 1H), 6.50-6.39 (m, 1H), 5.75-5.64 (m, 1H), 4.30- 4.17 (m, 3H), 3.97-3.86 (m, 3H), 3.84-3.76 (m, 1H), 1.92-1.82 (m, 1H), 1.05-0.97 (m, 2H), 0.90- 0.84 (m, 2H), 0.80 (br s, 4H) | B |
| | 2-(cyclopropyloxy)-N-{6-[(5- | | |
| | cyclopropyl-1H-pyrazol-3- | | |
| | yl)amino]-5-fluoro-4-methoxy-1,2- | | |
| | benzoxazol-3-yl}-6- | | |
| | methoxybenzene-1-sulfonamide | | |
| 81 | 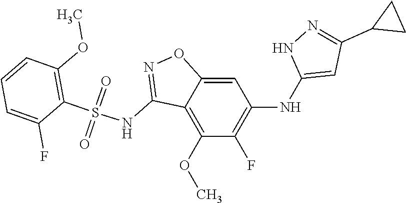 [SMILES: COc1cccc(F)c1S(=O)(=O)Nc1noc2cc(Nc3cc(C4CC4)n[nH]3)c(F)c(OC)c12] | LCMS m/z 492 [M + H]⁺; ¹H NMR (400 MHz, METHANOL-d₄) δ 7.49-7.41 (m, 1H), 7.39-7.31 (m, 1H), 6.90 (d, J = 8.3 Hz, 1H), 6.77 (dd, J = 10.2, 8.6 Hz, 1H), 5.72 (s, 1H), 4.13 (d, J = 2.3 Hz, 3H), 3.81 (s, 3H), 1.92-1.87 (m, 1H), 1.00-0.92 (m, 2H), 0.77-0.69 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-2-fluoro-6- | | |
| | methoxybenzene-1-sulfonamide | | |
| 82 | 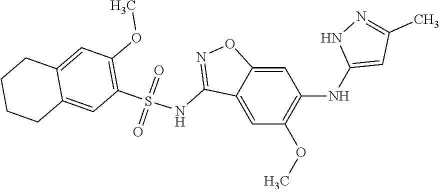 [SMILES: COc1cc2c(NS(=O)(=O)c3cc4c(cc3OC)CCCC4)noc2cc1Nc1cc(C)n[nH]1] | LCMS m/z 484 [M + H]⁺; ¹H NMR (400 MHz, METHANOL-d₄) δ 7.63-7.58 (m, 1H), 7.56 (s, 1H), 7.27-7.22 (m, 1H), 6.78 (s, 1H), 5.88 (s, 1H), 3.99-3.92 (m, 3H), 3.84-3.78 (m, 3H), 2.76 (br s, 2H), 2.72- 2.63 (m, 2H), 2.30-2.24 (m, 3H), 1.75 (br t, J = 3.0 Hz, 4H) | |
| | 3-methoxy-N-{5-methoxy-6-[(3- | | |
| | methyl-1H-pyrazol-5-yl)amino]- | | |
| | 1,2-benzoxazol-3-yl}-5,6,7,8- | | |
| | tetrahydronaphthalene-2- | | |
| | sulfonamide | | |
| 83 | 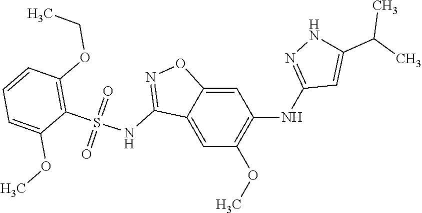 [SMILES: CCOc1cccc(OC)c1S(=O)(=O)Nc1noc2cc(Nc3cc(C(C)C)[nH]n3)c(OC)cc12] | LCMS m/z 502 [M + H]⁺; ¹H NMR (400 MHz, DMSO-d₆) δ 11.90 (s, 1H), 10.92 (br s, 1H), 8.16 (s, 1H), 8.09 (s, 1H), 7.43 (t, J = 8.3 Hz, 1H), 7.38 (s, 1H), 6.72 (dd, J = 8.5, 4.0 Hz, 2H), 5.87 (s, 1H), 4.05 (q, J = 7.0 Hz, 2H), 3.86 (s, 3H), 3.75 (s, 3H), 2.91-2.87 (m, 1H), 1.22-1.17 (m, 9H) | B |
| | 2-ethoxy-6-methoxy-N-(5- | | |
| | methoxy-6-{[5-(propan-2-yl)-1H- | | |
| | pyrazol-3-yl]amino}-1,2- | | |
| | benzoxazol-3-yl)benzene-1- | | |
| | sulfonamide | | |
| 84 | 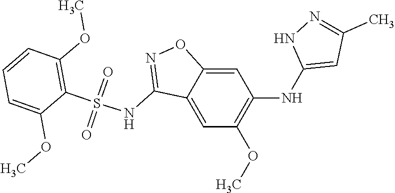 [SMILES: COc1cc2c(NS(=O)(=O)c3c(OC)cccc3OC)noc2cc1Nc1cc(C)n[nH]1] | LCMS m/z 460 [M + H]⁺; ¹H NMR (500 MHz, METHANOL-d₄) δ 7.60 (br s, 1H), 7.46 (t, J = 8.5 Hz, 1H), 7.31 (s, 1H), 6.72 (d, J = 8.5 Hz, 2H), 5.88 (s, 1H), 3.95 (s, 3H), 3.84 (s, 6H), 2.28 (s, 3H) | D |
| | 2,6-dimethoxy-N-{5-methoxy-6- | | |
| | [(3-methyl-1H-pyrazol-5-yl)amino]- | | |
| | 1,2-benzoxazol-3-yl}benzene-1- | | |
| | sulfonamide | | |
| 85 | 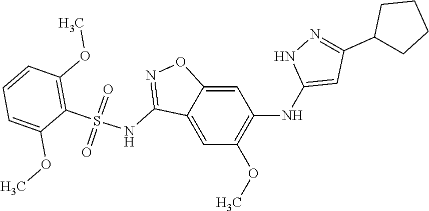 [SMILES: COc1cc2c(NS(=O)(=O)c3c(OC)cccc3OC)noc2cc1Nc1cc(C2CCCC2)n[nH]1] | LCMS m/z 514 [M + H]⁺; ¹H NMR (400 MHz, DMSO-d₆) δ 11.88 (s, 1H), 10.91 (s, 1H), 8.14 (br s, 1H), 8.04 (s, 1H), 7.46 (t, J = 8.5 Hz, 1H), 7.39 (s, 1H), 6.74 (d, J = 8.5 Hz, 2H), 5.83 (s, 1H), 3.87 (s, 3H), 3.76 (s, 6H), 3.11-2.92 (m, 1H), 2.14- 1.88 (m, 2H), 1.75-1.66 (m, 2H), 1.63-1.51 (m, 4H) | B |
| | N-{6-[(3-cyclopentyl-1H-pyrazol-5- | | |
| | yl)amino]-5-methoxy-1,2- | | |
| | benzoxazol-3-yl}-2,6- | | |
| | dimethoxybenzene-1-sulfonamide | | |
| 86 | 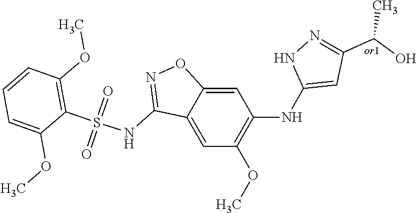 [SMILES: COc1cc2c(NS(=O)(=O)c3c(OC)cccc3OC)noc2cc1Nc1cc([C@H](C)O)n[nH]1] | SFC MS m/z 490 [M + H]⁺; [α]_{D}²² = −14.2° (C 0.1, CHCl₃); ¹H NMR (400 MHz, DMSO-d₆) δ 12.01 (s, 1H), 10.98 (br s, 1H), 8.20-8.14 (m, 2H), 7.52-7.45 (m, 1H), 6.80 (s, 1H), 6.77 (d, J = 8.6 Hz, 2H), 6.01 (s, 1H), 5.33 (br d, J = 4.9 Hz, 1H), 4.80-4.79 (m, 1H), 3.93 (s, 3H), 3.81 (s, 6H), 1.43 (d, J = 6.5 Hz, 3H) Column: ChiralPak ® AD (100 × 4.6 mm, 3 micron) Mobile phase A: CO₂ Mobile phase B: Methanol | B |
| | N-(6-{[3-(1-hydroxyethyl)-1H- | with 10 mM NH₃; 30% B | |
| | pyrazol-5-yl]amino}-5-methoxy- | isocratic, 160 bar, 4.0 mL/min; | |
| | 1,2-benzoxazol-3-yl)-2,6- | Rₜ = 6.65 min | |
| | dimethoxybenzene-1-sulfonamide | | |
| | or1 = single enantiomer absolute | | |
| | stereochemistry unknown | | |
| 87 | 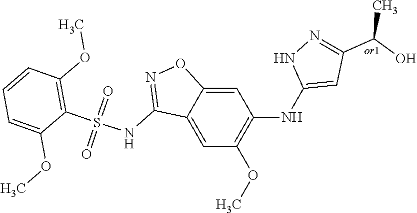 [SMILES: COc1cc2c(NS(=O)(=O)c3c(OC)cccc3OC)noc2cc1Nc1cc([C@@H](C)O)n[nH]1] | SFC MS m/z 490 [M + H]⁺; [α]_{D}²² = +33.8° (C 0.1, CHCl₃); ¹H NMR (400 MHz, DMSO-d₆) δ 11.99 (s, 1H), 10.94 (br s, 1H), 8.35-8.06 (m, 2H), 7.46 (t, J = 8.5 Hz, 1 H), 7.07 (s, 1H), 6.77 (d, J = 8.6 Hz, 2H), 6.00 (s, 1H), 5.33 (br d, J = 4.9 Hz, 1H), 4.80-4.76 (m, 1H), 3.92 (s, 3H), 3.78 (s, 6H), 1.43 (d, J = 6.5 Hz, 3H) Column: ChiralPak ® AD (100 × 4.6 mm, 3 micron) Mobile phase A: CO₂ Mobile phase B: Methanol | B |
| | N-(6-{[3-(1-hydroxyethyl)-1H- | with 10 mM NH₃; 30% B | |
| | pyrazol-5-yl]amino}-5-methoxy- | isocratic, 160 bar, 4.0 mL/min; | |
| | 1,2-benzoxazol-3-yl)-2,6- | Rₜ = 8.87 min | |
| | dimethoxybenzene-1-sulfonamide | | |
| | or1 = single enantiomer absolute | | |
| | stereochemistry unknown | | |
| 88 | 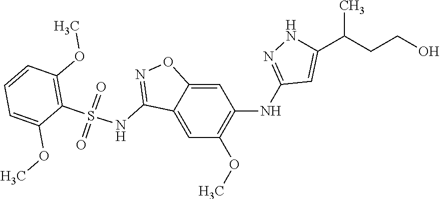 [SMILES: COc1cc2c(NS(=O)(=O)c3c(OC)cccc3OC)noc2cc1Nc1cc(C(C)CCO)[nH]n1] | LCMS m/z 518 [M + H]⁺; ¹H NMR (600 MHz, DMSO-d₆) δ 7.23 (t, J = 8.3 Hz, 1H), 7.03 (s, 1H), 7.00 (s, 1H), 6.60 (d, J = 8.4 Hz, 2H), 5.48 (s, 1H), 3.78 (s, 3H) 3.61 (s, 7H), 3.39 (q, J = 6.6 Hz, 3H), 2.80-2.69 (m, 1H), 1.72-1.53 (m, 2H), 1.11 (br d, J = 6.9 Hz, 3H). This is the H- NMR of the sodium salt | B |
| | N-(6-{[5-(4-hydroxybutan-2-yl)-1H- | | |
| | pyrazol-3-yl]amino}-5-methoxy- | | |
| | 1,2-benzoxazol-3-yl)-2,6- | | |
| | dimethoxybenzene-1-sulfonamide | | |
| 89 | 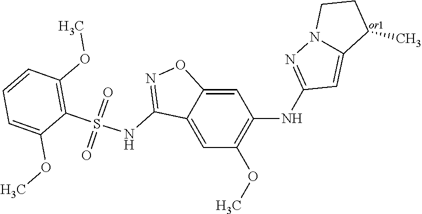 [SMILES: COc1cc2c(NS(=O)(=O)c3c(OC)cccc3OC)noc2cc1Nc1cc2n(n1)CC[C@@H]2C] | LCMS m/z 500 [M + H]⁺ ¹H NMR (400 MHz, DMSO-d₆) δ 10.93 (s, 1H), 8.07 (br s, 2H), 7.40 (br t, J = 8.2 Hz, 1H), 7.32 (br s, 1H), 6.71 (d, J = 8.4 Hz, 2H), 5.83 (s, 1H), 4.12-4.05 (m, 1H), 3.99-3.93 (m, 1H), 3.86 (s, 3H), 3.72 (s, 6H), 3.26-3.16 (m, 1H), 2.71-2.63 (m, 1H), 2.07- 2.00 (m, 1H), 1.23 (d, J = 7.0 Hz, 3H) The PMB protected enantiomer was separated by chiral SFC: Column: Chiralcel OJ-H SFC | B |
| | 2,6-dimethoxy-N-(5-methoxy-6- | (21 × 250 mm, 5 micron); Mobile | |
| | {[4-methyl-5,6-dihydro-4H- | phase A: CO₂; Mobile phase B: | |
| | pyrrolo[1,2-b]pyrazol-2-yl]amino}- | Methanol + 10 mM NH₃; 22% B | |
| | 1,2-benzoxazol-3-yl)benzene-1- | isocratic, 120 bar, 80 mL/min; | |
| | sulfonamide | Peak 2. | |
| | or1 = single enantiomer absolute | [α]_{D}²² = −10.4° (C 0.1, MeOH) | |
| | stereochemistry unknown | | |
| 90 | 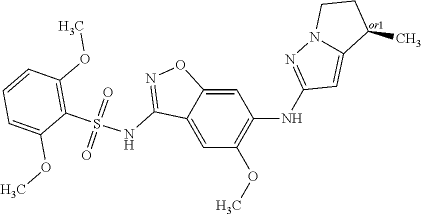 [SMILES: COc1cc2c(NS(=O)(=O)c3c(OC)cccc3OC)noc2cc1Nc1cc2n(n1)CC[C@H]2C] | LCMS m/z 500 [M + H]⁺; ¹H NMR (400 MHz, DMSO-d₆) δ 10.93 (br s, 1H), 8.08 (s, 2H), 7.41 (br t, J = 8.4 Hz, 1H), 7.33 (br s, 1H), 6.71 (d, J = 8.4 Hz, 2H), 5.84 (s, 1H), 4.13-4.05 (m, 1H), 4.01-3.90 (m, 1H), 3.87 (s, 3H), 3.72 (s, 6H), 3.27-3.16 (m, 1H), 2.71-2.63 (m, 1H) 2.07- 2.00 (m, 1H), 1.23 (d, J = 6.8 Hz, 3H) The PMB protected enantiomer was separated by chiral SFC: Column: Chiralcel OJ-H SFC | B |
| | 2,6-dimethoxy-N-(5-methoxy-6- | (21 × 250 mm, 5 micron); Mobile | |
| | {[4-methyl-5,6-dihydro-4H- | phase A: CO₂; Mobile phase B: | |
| | pyrrolo[1,2-b]pyrazol-2-yl]amino}- | Methanol + 10 mM NH₃; 22% B | |
| | 1,2-benzoxazol-3-yl)benzene-1- | isocratic, 120 bar, 80 mL/min; | |
| | sulfonamide | Peak 1. | |
| | or1 = single enantiomer absolute | [α]_{D}²² = +6.9° (C 0.1, MeOH) | |
| | stereochemistry unknown | | |
| 91 | 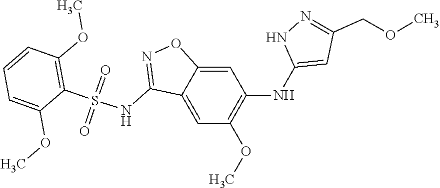 [SMILES: COCc1cc(Nc2cc3onc(NS(=O)(=O)c4c(OC)cccc4OC)c3cc2OC)[nH]n1] | LCMS m/z 490 [M + H]⁺; ¹H NMR (400 MHz, CDCl₃) δ 7.70 (s, 1H), 7.41 (s, 1H), 7.33 (t, J = 8.5 Hz, 1H), 7.07 (br s, 1H), 6.58 (d, J = 8.6 Hz, 2H), 5.99 (br s, 1H), 4.45 (s, 2H), 3.92 (s, 3H), 3.85 (s, 6H), 3.36 (s, 3H) | B |
| | 2,6-dimethoxy-N-(5-methoxy-6- | | |
| | {[3-(methoxymethyl)-1H-pyrazol-5- | | |
| | yl]amino}-1,2-benzoxazol-3- | | |
| | yl)benzene-1-sulfonamide | | |
| 92 | 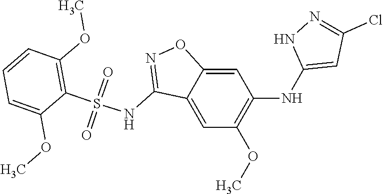 [SMILES: COc1cc2c(NS(=O)(=O)c3c(OC)cccc3OC)noc2cc1Nc1cc(Cl)n[nH]1] | LCMS m/z 480 [M + H]⁺; ¹H NMR (400 MHz, DMSO-d₆) δ 10.99 (s, 1H), 8.31 (s, 1H), 7.51- 7.47 (m, 2H), 6.74 (d, J = 8.5 Hz, 2H), 6.23 (s, 1H), 5.25 (s, 1H), 3.89 (s, 3H), 3.77 (s, 6H) | B |
| | N-{6-[(3-chloro-1H-pyrazol-5- | | |
| | yl)amino]-5-methoxy-1,2- | | |
| | benzoxazol-3-yl}-2,6- | | |
| | dimethoxybenzene-1-sulfonamide | | |
| 93 | 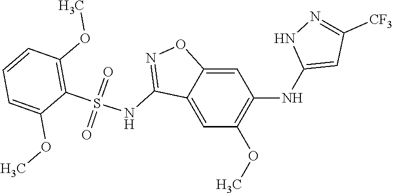 [SMILES: COc1cc2c(NS(=O)(=O)c3c(OC)cccc3OC)noc2cc1Nc1cc(C(F)(F)F)n[nH]1] | LCMS m/z 514 [M + H]⁺; ¹H NMR (600 MHz, DMSO-d₆) δ 12.04 (br s, 2H), 7.43 (s, 1H), 7.38 (t, J = 8.4 Hz, 1H), 6.66 (d, J = 8.4 Hz, 2H), 6.53 (s, 1H), 5.45 (s, 2H), 3.82 (s, 3H), 3.68 (s, 6H) | B |
| | 2,6-dimethoxy-N-(5-methoxy-6- | | |
| | {[3-(trifluoromethyl)-1H-pyrazol-5- | | |
| | yl]amino}-1,2-benzoxazol-3- | | |
| | yl)benzene-1-sulfonamide | | |
| 94 | 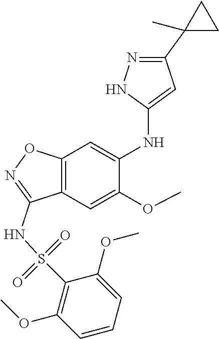 [SMILES: COc1cc2c(NS(=O)(=O)c3c(OC)cccc3OC)noc2cc1Nc1cc(C2(C)CC2)n[nH]1] | LCMS m/z 500 [M + H]⁺; ¹H NMR (600 MHz, DMSO-d₆) δ 10.94 (s, 1H), 8.08 (br s, 1H), 8.06 (s, 1H), 7.45 (t, J = 8.4 Hz, 1H), 7.41 (s, 1H), 6.73 (d, J = 8.5 Hz, 2H), 5.87 (br s, 1H), 3.87 (s, 3H), 3.76 (s, 6H), 1.37 (s, 3H), 0.90-0.86 (m, 2H), 0.77-0.73 (m, 2H) | B |
| | 2,6-dimethoxy-N-(5-methoxy-6- | | |
| | {[3-(1-methylcyclopropyl)-1H- | | |
| | pyrazol-5-yl]amino}-1,2- | | |
| | benzoxazol-3-yl)benzene-1- | | |
| | sulfonamide | | |
| 95 | 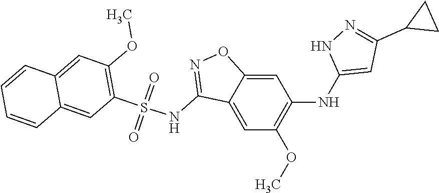 [SMILES: COc1cc2c(NS(=O)(=O)c3cc4ccccc4cc3OC)noc2cc1Nc1cc(C2CC2)n[nH]1] | LCMS m/z 506 [M + H]⁺; ¹H NMR (600 MHz, DMSO-d₆) δ 11.02 (s, 1H), 8.53 (s, 1H), 8.04 (d, J = 8.0 Hz, 1H), 7.94 (s, 1H), 7.86 (d, J = 8.4 Hz, 1H), 7.74 (br s, 1H), 7.61 (t, J = 7.7 Hz, 1H), 7.51 (s, 1H), 7.46 (t, J = 7.7 Hz, 1H), 7.32 (s, 1H), 5.76 (s, 1H), 3.92 (s, 3H), 3.87 (s, 3H), 1.88- 1.83 (m, 1H), 0.92-0.89 (m, 2H), 0.68-0.65 (m, 2 H) | B |
| | N-{6-[(3-cyclopropyl-1H-pyrazol-5- | | |
| | yl)amino]-5-methoxy-1,2- | | |
| | benzoxazol-3-yl}-3- | | |
| | methoxynaphthalene-2- | | |
| | sulfonamide | | |
| 96 | 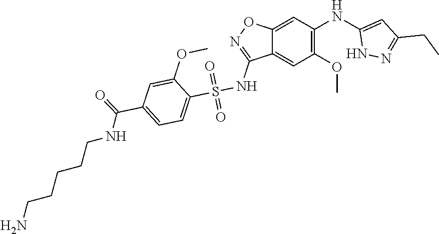 [SMILES: CCc1cc(Nc2cc3onc(NS(=O)(=O)c4ccc(C(=O)NCCCCCN)cc4OC)c3cc2OC)[nH]n1] | LCMS m/z 572 [M + H]⁺; ¹H NMR (400 MHz, METHANOL-d₄) δ 8.01 (d, J = 7.9 Hz, 1H), 7.53 (s, 1H), 7.47 (s, 1H), 7.39 (d, J = 9.2 Hz, 1H), 7.25 (s, 1H), 5.90 (s, 1H), 3.97 (s, 3H), 3.90 (s, 3H), 3.42-3.38 (m, 2H), 2.94-2.88 (m, 2H), 2.65 (q, J = 7.6 Hz, 2H), 1.73-1.62 (m, 4H), 1.45 (br d, J = 6.6 Hz, 2H), 1.29-1.25 (m, 3H) | D |
| | N-(5-aminopentyl)-4-({6-[(3-ethyl- | | |
| | 1H-pyrazol-5-yl)amino]-5-methoxy- | | |
| | 1,2-benzoxazol-3-yl}sulfamoyl)-3- | | |
| | methoxybenzamide | | |
| 97 | 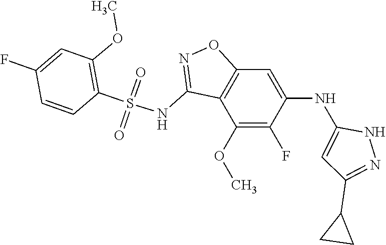 [SMILES: COc1cc(F)ccc1S(=O)(=O)Nc1noc2cc(Nc3cc(C4CC4)n[nH]3)c(F)c(OC)c12] | LCMS m/z 492 [M + H]⁺; ¹H NMR (400 MHz, METHANOL-d₄) δ 7.97 (dd, J = 8.8, 6.5 Hz, 1H), 7.49-7.39 (m, 1H), 6.98 (dd, J = 10.9, 2.3 Hz, 1H), 6.83 (td, J = 8.5, 2.3 Hz, 1H), 5.72 (s, 1H), 4.16 (d, J = 3.6 Hz, 3H), 3.89 (s, 3H), 1.94- 1.83 (m, 1H), 1.04-0.91 (m, 2 H) 0.78-0.63 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-4-fluoro-2- | | |
| | methoxybenzene-1-sulfonamide | | |
| 98 | 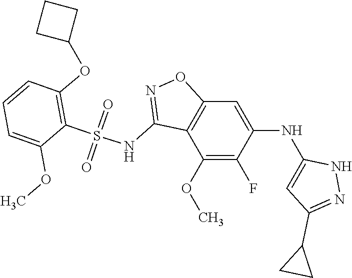 [SMILES: COc1cccc(OC2CCC2)c1S(=O)(=O)Nc1noc2cc(Nc3cc(C4CC4)n[nH]3)c(F)c(OC)c12] | LCMS m/z 544 [M + H]⁺; ¹H NMR (400 MHz, CHLOROFORM-d) δ 7.46 (d, J = 5.5 Hz, 1H), 7.34 (t, J = 8.4 Hz, 1H), 6.59 (d, J = 8.4 Hz, 1H), 6.50 (d, J = 4.5 Hz, 1H), 6.41 (d, J = 8.4 Hz, 1H), 5.70 (s, 1H), 4.71 (quin, J = 7.2 Hz, 1H), 4.23 (d, J = 3.9 Hz, 3H), 3.90 (s, 3H), 2.46-2.36 (m, 2H), 2.30-2.15 (m, 2H), 1.94-1.79 (m, 3H), 1.73-1.58 (m, 2H), 1.05-0.98 (m, 2H), 0.77 - 0.71 (m, 2H) | B |
| | 2-(cyclobutyloxy)-N-{6-[(3- | | |
| | cyclopropyl-1H-pyrazol-5- | | |
| | yl)amino]-5-fluoro-4-methoxy-1,2- | | |
| | benzoxazol-3-yl}-6- | | |
| | methoxybenzene-1-sulfonamide | | |
| 99 | 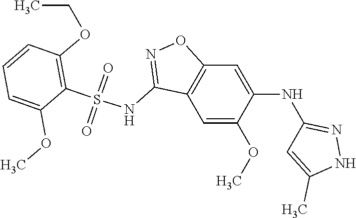 [SMILES: CCOc1cccc(OC)c1S(=O)(=O)Nc1noc2cc(Nc3cc(C)[nH]n3)c(OC)cc12] | LCMS m/z 474 [M + H]⁺; ¹H NMR (400 MHz, DMSO-d₆) δ 11.86 (s, 1H), 10.93 (s, 1H), 8.17 (s, 1H), 8.11 (s, 1H), 7.44 (t, J = 8.4 Hz, 1H), 7.39 (s, 1H), 6.73 (dd, J = 8.4, 4.1 Hz, 2H), 5.87 (s, 1H), 4.06 (q, J = 6.9 Hz, 2H), 3.87 (s, 3H), 3.76 (s, 3H), 2.19 (s, 3H), 1.20 (t, J = 7.0 Hz, 3H) | B |
| | 2-ethoxy-6-methoxy-N-{5- | | |
| | methoxy-6-[(5-methyl-1H-pyrazol- | | |
| | 3-yl)amino]-1,2-benzoxazol-3- | | |
| | yl}benzene-1-sulfonamide | | |
| 100 | 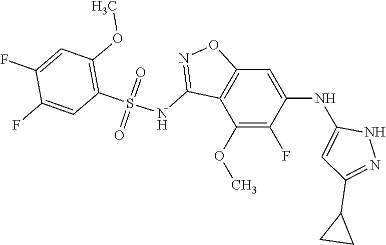 [SMILES: COc1cc(F)c(F)cc1S(=O)(=O)Nc1noc2cc(Nc3cc(C4CC4)n[nH]3)c(F)c(OC)c12] | LCMS m/z 510 [M + H]⁺; ¹H NMR (400 MHz, METHANOL-d₄) δ 7.86 (t, J = 9.4 Hz, 1H), 7.46-7.33 (m, 1H), 7.14 (dd, J = 12.1, 6.1 Hz, 1H), 5.72 (s, 1H), 4.15 (d, J = 2.9 Hz, 3H), 3.85 (s, 3H), 1.93-1.85 (m, 1H), 1.02-0.93 (m, 2H), 0.77-0.68 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-4,5-difluoro-2- | | |
| | methoxybenzene-1-sulfonamide | | |
| 101 | 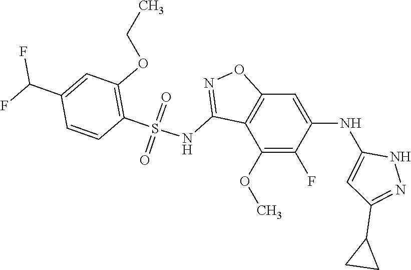 [SMILES: CCOc1cc(C(F)F)ccc1S(=O)(=O)Nc1noc2cc(Nc3cc(C4CC4)n[nH]3)c(F)c(OC)c12] | LCMS m/z 538.2 [M + H]⁺; ¹H NMR (400 MHz, CHLOROFORM-d₆) δ 8.14 (d, J = 8.1 Hz, 1H), 7.29 (d, J = 5.5 Hz, 1H), 7.12 (d, J = 7.9 Hz, 1H), 7.03 (s, 1H), 6.71-6.36 (m, 2H), 5.63 (s, 1H), 4.20-4.11 (m, 5H), 1.81-1.73 (m, 1H), 1.44 (t, J = 6.9 Hz, 3H), 0.96-0.90 (m, 2H), 0.68-0.62 (m, 2H) | B |
| | N-{6-[(3-cyclopropyl-1H-pyrazol-5- | | |
| | yl)amino]-5-fluoro-4-methoxy-1,2- | | |
| | benzoxazol-3-yl}-4- | | |
| | (difluoromethyl)-2-ethoxybenzene- | | |
| | 1-sulfonamide | | |
| 102 | 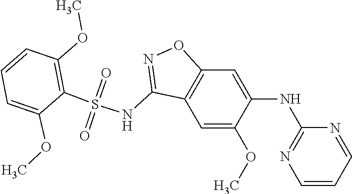 [SMILES: COc1cc2c(NS(=O)(=O)c3c(OC)cccc3OC)noc2cc1Nc1ncccn1] | LCMS m/z 458 [M + H]⁺; ¹H NMR (400 MHz, DMSO-d₆) δ 8.64 (s, 1H), 8.61 (d, J = 4.8 Hz, 2H), 8.29 (s, 1H), 7.55 (t, J = 8.5 Hz, 1H), 7.32 (d, J = 8.6 Hz, 2H), 7.16 (s, 1H), 7.03 (t, J = 4.8 Hz, 1H), 6.85-6.89 (m, 2H), 6.80 (d, J = 8.5 Hz, 2H), 3.84 (s, 3H), 3.75 (s, 6H), 3.70 (s, 3H) | B |
| | 2,6-dimethoxy-N-{5-methoxy-6- | | |
| | [(pyrimidin-2-yl)amino]-1,2- | | |
| | benzoxazol-3-yl}benzene-1- | | |
| | sulfonamide | | |
| 103 | 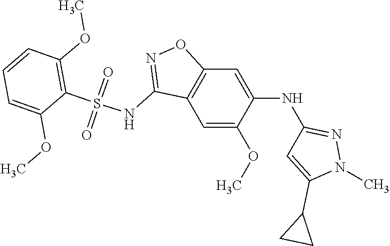 [SMILES: COc1cc2c(NS(=O)(=O)c3c(OC)cccc3OC)noc2cc1Nc1cc(C2CC2)n(C)n1] | LCMS m/z 500 [M + H]⁺; ¹H NMR (400 MHz, DMSO-d₆) δ 10.93 (s, 1H), 8.09 (s, 1H), 8.02 (s, 1H), 7.47 (t, J = 8.4 Hz, 1H), 7.39 (s, 1H), 6.75 (d, J = 8.5 Hz, 2H), 5.70 (s, 1H), 3.87 (s, 3H), 3.77 (s, 3H), 3.76 (s, 6H), 1.90- 1.82 (m, 1H), 0.98-0.92 (m, 2H), 0.61-0.56 (m, 2H) | B |
| | N-{6-[(5-cyclopropyl-1-methyl-1H- | | |
| | pyrazol-3-yl)amino]-5-methoxy- | | |
| | 1,2-benzoxazol-3-yl}-2,6- | | |
| | dimethoxybenzene-1-sulfonamide | | |
| 104 | 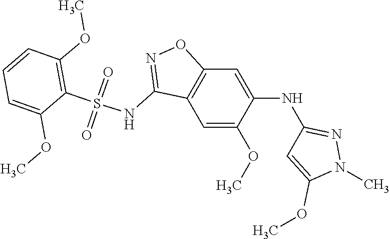 [SMILES: COc1cc2c(NS(=O)(=O)c3c(OC)cccc3OC)noc2cc1Nc1cc(OC)n(C)n1] | LCMS m/z 490 [M + H]⁺; ¹H NMR (400 MHz, DMSO-d₆) δ 10.95 (s, 1H), 8.17 (s, 1H), 8.13 (s, 1H), 7.47 (t, J = 8.4 Hz, 1H), 7.41 (s, 1H), 6.75 (d, J = 8.5 Hz, 2H), 5.55 (s, 1H), 3.88 (s, 3H), 3.85 (s, 3H), 3.77 (s, 6H) | B |
| | 2,6-dimethoxy-N-{5-methoxy-6- | | |
| | [(5-methoxy-1-methyl-1H-pyrazol- | | |
| | 3-yl)amino]-1,2-benzoxazol-3- | | |
| | yl}benzene-1-sulfonamide | | |
| 105 | 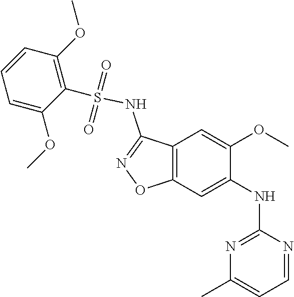 [SMILES: COc1cc2c(NS(=O)(=O)c3c(OC)cccc3OC)noc2cc1Nc1nccc(C)n1] | LCMS m/z 472 [M + H]⁺; ¹H NMR (400 MHz, DMSO-d₆) δ 11.12 (s, 1H), 8.66 (br s, 1H), 8.46 (d, J = 5.1 Hz, 1H), 8.15 (s, 1H), 7.56 (br s, 1H), 7.46 (br t, J = 8.4 Hz, 1H), 6.91 (d, J = 5.1 Hz, 1H), 6.74 (d, J = 8.3 Hz, 2H), 3.93 (s, 3H), 3.75 (s, 6H), 2.43 (s, 3H) | B |
| | 2,6-dimethoxy-N-{5-methoxy-6- | | |
| | [(4-methylpyrimidin-2-yl)amino]- | | |
| | 1,2-benzoxazol-3-yl}benzene-1- | | |
| | sulfonamide | | |
| 106 | 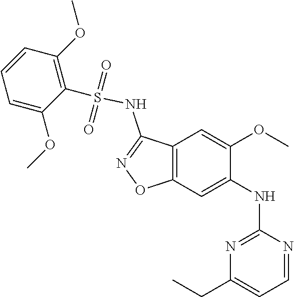 [SMILES: CCc1ccnc(Nc2cc3onc(NS(=O)(=O)c4c(OC)cccc4OC)c3cc2OC)n1] | LCMS m/z 486 [M + H]⁺; ¹H NMR (400 MHz, DMSO-d₆) δ 8.62 (s, 1H), 8.47 (d, J = 5.1 Hz, 1H), 8.10 (s, 1H), 7.48 (br s, 1H), 7.39 (br t, J = 8.3 Hz, 1H), 6.91 (d, J = 5.1 Hz, 1H), 6.70 (d, J = 8.5 Hz, 2H), 3.92 (s, 3H), 3.71 (s, 6H), 2.71 (q, J = 7.6 Hz, 2H), 1.92 (s, 1H), 1.26 (t, J = 7.6 Hz, 3H) | B |
| | N-{6-[(4-ethylpyrimidin-2- | | |
| | yl)amino]-5-methoxy-1,2- | | |
| | benzoxazol-3-yl}-2,6- | | |
| | dimethoxybenzene-1-sulfonamide | | |
| 107 | 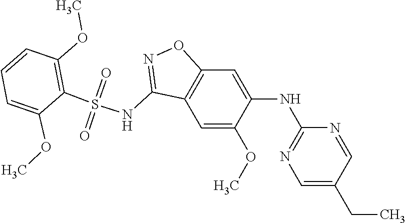 [SMILES: CCc1cnc(Nc2cc3onc(NS(=O)(=O)c4c(OC)cccc4OC)c3cc2OC)nc1] | LCMS m/z 486 [M + H]⁺; ¹H NMR (400 MHz, DMSO-d₆) δ 11.12 (br s, 1H), 8.57 (br d, J = 2.5 Hz, 1H), 8.50 (s, 2H), 8.13 (br s, 1H), 7.45-7.55 (m, 1H), 7.44-7.34 (m, 1H), 6.71 (br d, J = 8.0 Hz, 2H), 3.92 (s, 3H), 3.71 (br s, 6H), 2.59-2.53 (m, 2H), 1.20 (t, J = 7.6 Hz, 3H) | B |
| | N-{6-[(5-ethylpyrimidin-2- | | |
| | yl)amino]-5-methoxy-1,2- | | |
| | benzoxazol-3-yl}-2,6- | | |
| | dimethoxybenzene-1-sulfonamide | | |
| 108 | 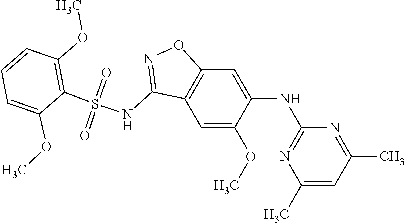 [SMILES: COc1cc2c(NS(=O)(=O)c3c(OC)cccc3OC)noc2cc1Nc1nc(C)cc(C)n1] | LCMS m/z 486 [M + H]⁺; ¹H NMR (400 MHz, DMSO-d₆) δ 11.14 (s, 1H), 8.64 (br s, 1H), 8.00 (br s, 1H), 7.45 (br s, 1H), 7.38 (t, J = 1.0 Hz, 1H), 6.78 (s, 1H), 6.70 (br d, J = 8.4 Hz, 2H), 3.92 (s, 3H), 3.70 (br s, 6H), 2.38 (s, 6H) | B |
| | N-{6-[(4,6-dimethylpyrimidin-2- | | |
| | yl)amino]-5-methoxy-1,2- | | |
| | benzoxazol-3-yl}-2,6- | | |
| | dimethoxybenzene-1-sulfonamide | | |
| 109 | 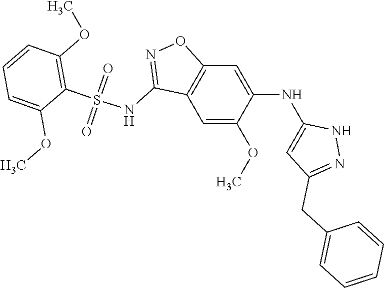 [SMILES: COc1cc2c(NS(=O)(=O)c3c(OC)cccc3OC)noc2cc1Nc1cc(Cc2ccccc2)n[nH]1] | LCMS m/z 536 [M + H]⁺; ¹H NMR (400 MHz, DMSO-d₆) δ 12.04 (s, 1H), 10.93 (s, 1H), 8.17 (s, 1H), 8.13 (s, 1H), 7.47 (t, J = 8.5 Hz, 1H), 7.40 (s, 1H), 7.34-7.19 (m, 5H), 6.74 (d, J = 8.5 Hz, 2H), 5.86 (d, J = 1.8 Hz, 1H), 3.92 (s, 2H), 3.86 (s, 3H), 3.76 (s, 6H) | B |
| | N-{6-[(3-benzyl-1H-pyrazol-5- | | |
| | yl)amino]-5-methoxy-1,2- | | |
| | benzoxazol-3-yl}-2,6- | | |
| | dimethoxybenzene-1-sulfonamide | | |
| 110 | 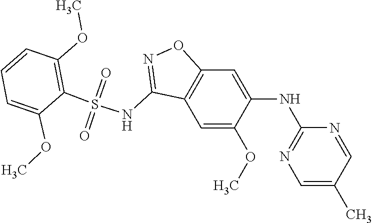 [SMILES: COc1cc2c(NS(=O)(=O)c3c(OC)cccc3OC)noc2cc1Nc1ncc(C)cn1] | LCMS m/z 472 [M + H]⁺; ¹H NMR (400 MHz, DMSO-d₆) δ 11.04 (br s, 1H), 8.52 (br s, 1H), 8.40 (s, 2H), 8.07 (s, 1H), 7.47 (br s, 1H), 7.37 (br t, J = 8.4 Hz, 1H), 6.66 (d, J = 8.4 Hz, 2H), 3.85 (s, 3H), 3.67 (s, 6H), 2.12 (s, 3H | B |
| | 2,6-dimethoxy-N-{5-methoxy-6- | | |
| | [(5-methylpyrimidin-2-yl)amino]- | | |
| | 1,2-benzoxazol-3-yl}benzene-1- | | |
| | sulfonamide | | |
| 111 | 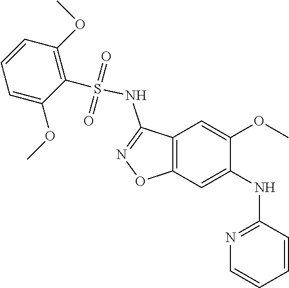 [SMILES: COc1cc2c(NS(=O)(=O)c3c(OC)cccc3OC)noc2cc1Nc1ccccn1] | LCMS m/z 457 [M + H]⁺; ¹H NMR (400 MHz, DMSO-d₆) δ 11.05 (br s, 1H), 8.75 (br s, 1H), 8.51 (br s, 1H), 8.27 (br d, J = 4.5 Hz, 1H), 7.67-7.60 (m, 1H), 7.46 (br d, J = 13.1 Hz, 1H), 7.27 (br d, J = 7.5 Hz, 1H), 6.87 (br t, J = 5.2 Hz, 1H), 6.73 (br d, J = 8.3 Hz, 2H), 3.91 (s, 3H), 3.75 (br s, 6H) | B |
| | 2,6-dimethoxy-N-{5-methoxy-6- | | |
| | [(pyridin-2-yl)amino]-1,2- | | |
| | benzoxazol-3-yl}benzene-1- | | |
| | sulfonamide | | |
| 112 | 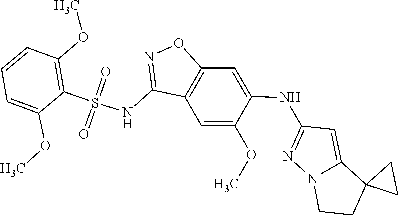 [SMILES: COc1cc2c(NS(=O)(=O)c3c(OC)cccc3OC)noc2cc1Nc1cc2n(n1)CCC21CC1] | LCMS m/z 512 [M + H]⁺; ¹H NMR (600 MHz, CD₃OD) δ 7.68 (s, 1H), 7.45 (br t, J = 8.3 Hz, 1H), 7.30 (s, 1H), 6.72 (br d, J = 8.4 Hz, 2H), 4.19 (br t, J = 7.1 Hz, 2H), 3.94 (s, 3H), 3.84 (s, 6H), 2.56 (br t, J = 7.1 Hz, 2H), 1.03 (br d, J = 12.2 Hz, 4H) | B |
| | N-{6-[(5′,6′- | | |
| | dihydrospiro[cyclopropane-1,4′- | | |
| | pyrrolo[1,2-b]pyrazol]-2′-yl)amino]- | | |
| | 5-methoxy-1,2-benzoxazol-3-yl}- | | |
| | 2,6-dimethoxybenzene-1- | | |
| | sulfonamide | | |
| 113 | 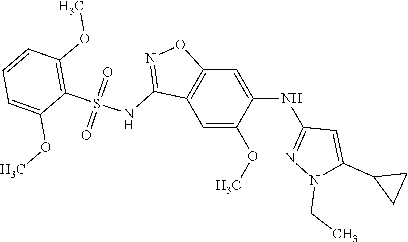 [SMILES: CCn1nc(Nc2cc3onc(NS(=O)(=O)c4c(OC)cccc4OC)c3cc2OC)cc1C1CC1] | LCMS m/z 514 [M + H]⁺; ¹H NMR (400 MHz, DMSO-d₆) δ 10.89 (s, 1H), 7.64 (s, 1H), 7.41- 7.37 (m, 1H), 6.66 (d, J = 8.5 Hz, 2H), 6.28 (s, 1H), 5.76 (s, 1H), 3.80 (s, 3H), 3.76 (q, J = 7.3 Hz, 2H), 3.69 (s, 6H), 1.79-1.74 (m, 1H), 1.12 (t, J = 7.2 Hz, 3H), 0.78-0.72 (m, 2H), 0.59-0.54 (m, 2H) | B |
| | N-{6-[(5-cyclopropyl-1-ethyl-1H- | | |
| | pyrazol-3-yl)amino]-5-methoxy- | | |
| | 1,2-benzoxazol-3-yl}-2,6- | | |
| | dimethoxybenzene-1-sulfonamide | | |
| 114 | 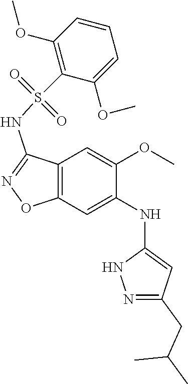 [SMILES: COc1cc2c(NS(=O)(=O)c3c(OC)cccc3OC)noc2cc1Nc1cc(CC(C)C)n[nH]1] | LCMS m/z 502 [M + H]⁺; ¹H NMR (400 MHz, DMSO-d₆) δ 11.86 (br s, 1H), 10.93 (s, 1H), 8.15 (br s, 1H), 8.08 (s, 1H), 7.47 (t, J = 8.4 Hz, 1H), 7.41 (s, 1H), 6.75 (d, J = 8.6 Hz, 2H), 5.88 (s, 1H), 3.88 (s, 3H), 3.77 (s, 6H), 1.94-1.81 (m, 1H), 0.90 (d, J = 6.6 Hz, 6H) | B |
| | 2,6-dimethoxy-N-(5-methoxy-6- | | |
| | {[3-(2-methylpropyl)-1H-pyrazol-5- | | |
| | yl]amino}-1,2-benzoxazol-3- | | |
| | yl)benzene-1-sulfonamide | | |
| 115 | 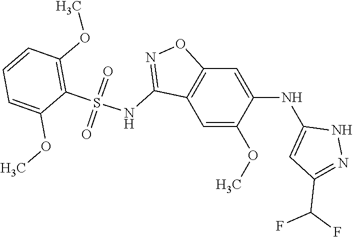 [SMILES: COc1cc2c(NS(=O)(=O)c3c(OC)cccc3OC)noc2cc1Nc1cc(C(F)F)n[nH]1] | LCMS m/z 496 [M + H]⁺; ¹H NMR (400 MHz, DMSO-d₆) δ 12.88 (s, 1H), 10.89 (br s, 1H), 8.11 (s, 1H), 7.45-7.36 (m, 2H), 7.07 (br t, J = 54.3 Hz, 1H), 6.67 (d, J = 8.5 Hz, 2H), 6.34 (s, 1H), 3.83 (s, 3H), 3.69 (s, 6H) | B |
| | N-(6-{[3-(difluoromethyl)-1H- | | |
| | pyrazol-5-yl]amino}-5-methoxy- | | |
| | 1,2-benzoxazol-3-yl)-2,6- | | |
| | dimethoxybenzene-1-sulfonamide | | |
| 116 | 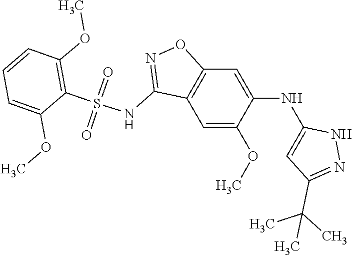 [SMILES: COc1cc2c(NS(=O)(=O)c3c(OC)cccc3OC)noc2cc1Nc1cc(C(C)(C)C)n[nH]1] | LCMS m/z 502 [M + H]⁺; ¹H NMR (400 MHz, DMSO-d₆) δ 11.90 (br s, 1H), 10.93 (s, 1H), 8.16 (s, 1H), 8.06 (s, 1H), 7.47 (t, J = 8.5 Hz, 1H), 7.40 (s, 1H), 6.75 (d, J = 8.5 Hz, 2H), 5.88 (s, 1H), 3.88 (s, 3H), 3.77 (s, 6H), 1.27 (s, 9H) | B |
| | N-{6-[(3-tert-butyl-1H-pyrazol-5- | | |
| | yl)amino]-5-methoxy-1,2- | | |
| | benzoxazol-3-yl}-2,6- | | |
| | dimethoxybenzene-1-sulfonamide | | |
| 117 | 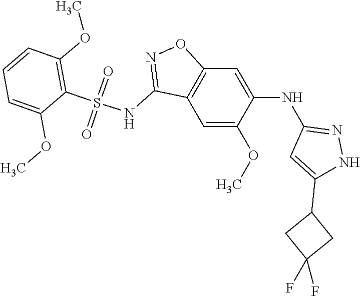 [SMILES: COc1cc2c(NS(=O)(=O)c3c(OC)cccc3OC)noc2cc1Nc1cc(C2CC(F)(F)C2)[nH]n1] | LCMS m/z 536 [M + H]⁺; ¹H NMR (DMSO-d₆, 400 MHz) 12.11 (s, 1H), 10.94 (s, 1H), 8.14 (s, 1H), 7.47 (t, J = 8.4 Hz, 1H), 7.42 (s, 1H), 6.75 (d, J = 8.5 Hz, 2H), 6.06 (s, 1H), 3.89 (s, 3H), 3.77 (s, 6H), 3.07-2.94 (m, 2H), 2.78-2.67 (m, 3H), 2.34- 2.33 (m, 1H) | B |
| | N-(6-{[5-(3,3-difluorocyclobutyl)- | | |
| | 1H-pyrazol-3-yl]amino}-5- | | |
| | methoxy-1,2-benzoxazol-3-yl)-2,6- | | |
| | dimethoxybenzene-1-sulfonamide | | |
| 118 | 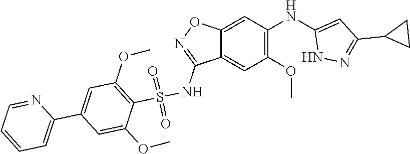 [SMILES: COc1cc2c(NS(=O)(=O)c3c(OC)cc(-c4ccccn4)cc3OC)noc2cc1Nc1cc(C2CC2)n[nH]1] | LCMS m/z 563 [M + H]⁺; ¹H NMR (400 MHz, METHANOL-d₄) δ 8.67-8.63 (m, 1H), 8.00-7.91 (m, 2H), 7.66 (br s, 1H), 7.47-7.42 (m, 1H), 7.38- 7.31 (m, 3H), 5.77 (s, 1H), 4.00- 3.96 (m, 9H), 1.96-1.86 (m, 1H), 1.02-0.96 (m, 2H), 0.79-0.72 (m, 2H) | |
| | N-{6-[(3-cyclopropyl-1H-pyrazol-5- | | |
| | yl)amino]-5-methoxy-1,2- | | |
| | benzoxazol-3-yl}-2,6-dimethoxy-4- | | |
| | (pyridin-2-yl)benzene-1- | | |
| | sulfonamide | | |
| 119 | 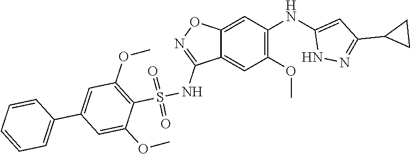 [SMILES: COc1cc2c(NS(=O)(=O)c3c(OC)cc(-c4ccccc4)cc3OC)noc2cc1Nc1cc(C2CC2)n[nH]1] | LCMS m/z 562 [M + H]⁺; ¹H NMR (400 MHz, CD₃OD) δ 7.62 (d, J = 7.3 Hz, 2H), 7.55- 7.49 (m, 1H), 7.46-7.41 (m, 2H), 7.40-7.35 (m, 1H), 7.29 (s, 1H), 6.87 (s, 2H), 5.75 (s, 1H), 3.93 (s, 3H), 3.84 (s, 6H), 1.92-1.85 (m, 1H), 0.99-0.93 (m, 2H), 0.75-0.71 (m, 2H) | E |
| | N-{6-[(3-cyclopropyl-1H-pyrazol-5- | | |
| | yl)amino]-5-methoxy-1,2- | | |
| | benzoxazol-3-yl}-3,5- | | |
| | dimethoxy[1,1′-biphenyl]-4- | | |
| | sulfonamide | | |
| 120 | 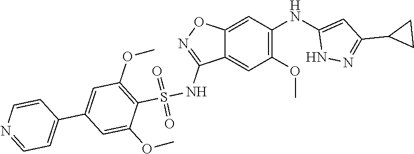 [SMILES: COc1cc2c(NS(=O)(=O)c3c(OC)cc(-c4ccncc4)cc3OC)noc2cc1Nc1cc(C2CC2)n[nH]1] | LCMS m/z 563 [M + H]⁺; ¹H NMR (400 MHz, DMSO-d₆) δ 11.89 (s, 1H), 11.09 (s, 1H), 8.70-8.65 (m, 2H), 8.13 (s, 1H), 8.06 (s, 1H), 7.82-7.79 (m, 2H), 7.42 (s, 1H), 7.08 (s, 2H), 5.78 (s, 1H), 3.89 (s, 9H), 1.90-1.79 (m, 1H), 0.94-0.86 (m, 2H), 0.69-0.62 (m, 2H) | E |
| | N-{6-[(3-cyclopropyl-1H-pyrazol-5- | | |
| | yl)amino]-5-methoxy-1,2- | | |
| | benzoxazol-3-yl}-2,6-dimethoxy-4- | | |
| | (pyridin-4-yl)benzene-1- | | |
| | sulfonamide | | |
| 121 | 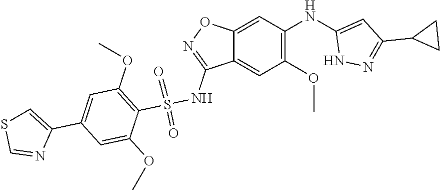 [SMILES: COc1cc2c(NS(=O)(=O)c3c(OC)cc(-c4cscn4)cc3OC)noc2cc1Nc1cc(C2CC2)n[nH]1] | LCMS m/z 569 [M + H]⁺; ¹H NMR (400 MHz, DMSO-d₆) δ 11.90 (br s, 1H), 11.02 (s, 1H), 9.22 (d, J = 1.8 Hz, 1H), 8.49 (d, J = 1.9 Hz, 1H), 8.12 (br s, 1H), 8.09-8.01 (m, 1H), 7.43(s, 1H), 7.33 (s, 2H), 5.77 (d, J = 7.5 Hz, 1H), 3.90-3.84 (m, 9H), 1.85 (ddd, J = 4.9, 8.4, 13.4 Hz, 1H), 0.98-0.90 (m, 2H), 0.71-0.63 (m, 2H) | E |
| | N-{6-[(3-cyclopropyl-1H-pyrazol-5- | | |
| | yl)amino]-5-methoxy-1,2- | | |
| | benzoxazol-3-yl}-2,6-dimethoxy-4- | | |
| | (1,3-thiazol-4-yl)benzene-1- | | |
| | sulfonamide | | |
| 122 | 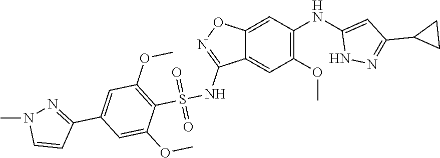 [SMILES: COc1cc2c(NS(=O)(=O)c3c(OC)cc(-c4ccn(C)n4)cc3OC)noc2cc1Nc1cc(C2CC2)n[nH]1] | LCMS m/z 566 [M + H]⁺; ¹H NMR (400 MHz, CDCl₃) δ 7.68 (s, 1H), 7.46 (s, 1H), 7.38 (d, J = 2.3 Hz, 1H), 7.01 (s, 3H), 6.53 (d, J = 2.4 Hz, 1H), 5.70 (s, 1H), 3.97 (s, 6H), 3.95 (s, 6H), 1.87-1.80 (m, 1H), 1.00-0.95 (m, 2H), 0.75-0.71 (m, 2H) | E |
| | N-{6-[(3-cyclopropyl-1H-pyrazol-5- | | |
| | yl)amino]-5-methoxy-1,2- | | |
| | benzoxazol-3-yl}-2,6-dimethoxy-4- | | |
| | (1-methyl-1H-pyrazol-3-yl)benzene- | | |
| | 1-sulfonamide | | |
| 123 | 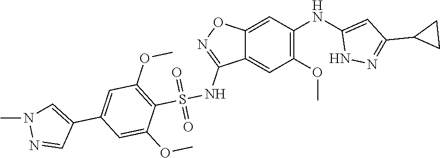 [SMILES: COc1cc2c(NS(=O)(=O)c3c(OC)cc(-c4cnn(C)c4)cc3OC)noc2cc1Nc1cc(C2CC2)n[nH]1] | LCMS m/z 566 [M + H]⁺; ¹H NMR (400 MHz, CDCl₃) δ 7.74 (d, J = 9.4 Hz, 2H), 7.63 (s, 1H), 7.46 (s, 1H), 6.98 (s, 1H), 6.63 (s, 2H), 5.69 (s, 1H), 3.98 (s, 3H), 3.94 (s, 3H), 3.93 (s, 6H), 1.90-1.82 (m, 1H), 1.02- 0.96 (m, 2H), 0.75-0.70 (m, 2H) | E |
| | N-{6-[(3-cyclopropyl-1H-pyrazol-5- | | |
| | yl)amino]-5-methoxy-1,2- | | |
| | benzoxazol-3-yl}-2,6-dimethoxy-4- | | |
| | (1-methyl-1H-pyrazol-4- | | |
| | yl)benzene-1-sulfonamide | | |

### Intermediate O: 5-Ethyl-1-{[2-(trimethylsilyl)ethoxy]methyl}-1H-pyrazol-3-amine

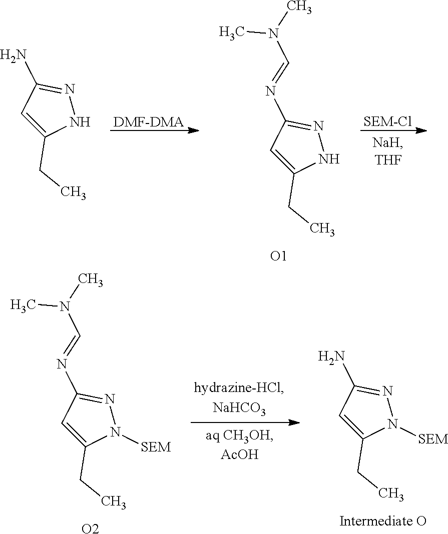

N-(5-ethyl-1H-pyrazol-3-yl)-N,N-dimethylmethanimidamide (O1): A solution of 5-ethyl-1H-pyrazol-3-amine in DMF-DMA was heated at 90° C. for 15 hrs after which time the reaction was cooled, concentrated and purified by silica gel chromatography (eluting with 0-10% methanol in DCM) to give 01 (4.2 g, 94%) as a viscous brown oil. LCMS m/z 167 [M+H]⁺.

N-(5-ethyl-1-{[2-(trimethylsilyl)ethoxy]methyl}-1H-pyrazol-3-yl)-N, N-dimethylmethanimidamide (O2): Sodium hydride (3.3 g, 81.6 mmol, 60% dispersion in mineral oil) was added in a portionwise manner to a stirred solution of N-(5-ethyl-1H-pyrazol-3-yl)-N,N-dimethylmethanimidamide (O1) (11.3 g, 67.9 mmol) in anhydrous THF (100 mL) at ˜0° C., and the reaction stirred for 30 mins. At this time, [2-(chloromethoxy)ethyl](trimethyl) silane (17.0 g, 18.0 mL, 102 mmol) was added, and the reaction stirred for two hrs at rt. LCMS indicated that most of the starting material was consumed, and the reaction was diluted with EtOAc (100 mL) and washed with saturated aq ammonium chloride solution (100 mL). The organic layer was separated, and the aqueous layer was extracted with EtOAc (3×50 mL). The combined organic layers were washed with brine (100 mL), dried over sodium sulfate, filtered and concentrated to give 02 (18.7 g, 93%) as a brown oil. LCMS m/z 297.1 [M+H]⁺.

5-Ethyl-1-{[2-(trimethylsilyl)ethoxy]methyl]-1H-pyrazol-3-amine (Intermediate O): Solid NaHCO₃ (17.1 g, 204 mmol) was added to a stirred solution of hydrazine hydrochloride (14.0 g, 204 mmol) in a mixture of methanol (140 mL)/H₂O (10 mL), and the reaction allowed to stir for 10 mins at rt. Next, N-(5-ethyl-1-{[2-(trimethylsilyl)ethoxy]methyl}-1H-pyrazol-3-yl)-N,N-dimethylmethanimidamide (O2) (20.2 g, 68.0 mmol) and acetic acid (4.1 g, 3.9 mL, 68.0 mmol) was added, and the reaction stirred at 50° C. for seven hrs. LCMS indicated the starting material was consumed, and the reaction was concentrated, diluted with EtOAc (200 mL), washed with saturated aq NaHCO₃ solution (100 mL) and brine (100 mL). The organic layer was dried over sodium sulfate, filtered, and concentrated to afford a residue that was purified by silica gel chromatography (eluting with 0-50% EtOAc in Pet. ether) to afford Intermediate O (5.3 g, 32%) as a yellow oil. LCMS m/z 242 [M+H]⁺; ¹H NMR (400 MHZ, CHLOROFORM-d) δ 5.49 (s, 1H), 5.20 (s, 2H), 3.60-3.53 (m, 2H), 2.61 (q, J=7.4 Hz, 2H), 1.24 (t, J=7.6 Hz, 3H), 0.93-0.86 (m, 2H, -0.01 (s, 9H).

### Intermediate P: N6-(5-ethyl-1-{[2-(trimethylsilyl)ethoxy]methyl}-1H-pyrazol-3-yl)-5-methoxy-1.2-benzoxazole-3,6-diamine

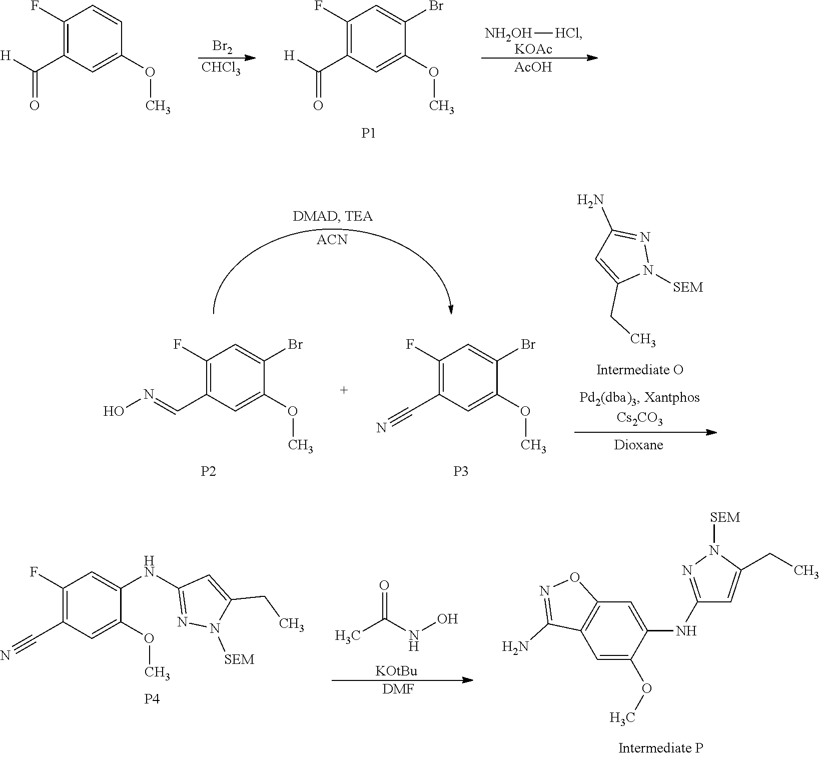

4-Bromo-2-fluoro-5-methoxybenzaldehyde (P1): To a cooled (0° C.) solution of 2-fluoro-5-methoxybenzaldehyde (25.0 g, 162.2 mmol) in chloroform (500 mL) was added bromine (51.8 g, 16.6 mL, 324.0 mmol) dropwise under a dry nitrogen atmosphere. After the addition was completed, the black solution was warmed to rt and stirred for 5 days. At this time, the red solution was poured into saturated aq NaHCO₃ solution (200 mL) and extracted with DCM (3×200 mL). The combined organic layers were washed with H₂O (100 mL), dried over sodium sulfate, filtered, concentrated, and purified by silica gel chromatography (eluting with 0-10% EtOAc in Pet. ether) to give 24 g of solid material, which was shown to be a 2:1 mixture of 4-bromo-2-fluoro-5-methoxybenzaldehyde (P1) and 2-fluoro-5-methoxybenzaldehyde. Trituration with Pet. ether (50 mL) followed by filtration afforded P1 (6.40 g, 17%) as a pale-yellow solid. 1H NMR (400 MHZ, CHLOROFORM-d) δ 10.39 (s, 1H), 7.23-6.95 (m, 2H), 3.95 (s, 3H).

N-[(E)-(4-bromo-2-fluoro-5-methoxyphenyl)methylidene]hydroxylamine (P2): To a suspension of 4-bromo-2-fluoro-5-methoxybenzaldehyde (P1) (6.4 g, 27.5 mmol) and potassium acetate (5.4 g, 54.9 mmol) in acetic acid (40 mL) was added hydroxylamine hydrochloride (3.8 g, 54.9 mmol). The reaction was then refluxed at 120° C. for 4 hrs after which LCMS indicated the starting material was consumed, and two new products detected. The reaction was cooled, concentrated and diluted with saturated aq NaHCO₃ solution (200 mL). The mixture was extracted with EtOAc (3×50 mL), and the organic extracts dried over sodium sulfate, filtered, concentrated, and purified by silica gel chromatography (eluting with 0-10% EtOAc in Pet. ether) to afford 4-bromo-2-fluoro-5-methoxybenzonitrile (P3, 2.0 g, 32%) as a white solid (see following experiment for analytical data) followed by the more polar P2 (4.0 g, 59%) also as a white solid. For P2, ¹H NMR (400 MHZ, CHLOROFORM-d) δ 8.24 (s, 1H), 7.27 (d, J=9.1 Hz, 1H), 7.22-7.16 (m, 1H), 3.84 (s, 3H).

4-Bromo-2-fluoro-5-methoxybenzonitrile (P3): To a solution of N-[(E)-(4-bromo-2-fluoro-5-methoxyphenyl)methylidene]hydroxylamine (P2) (3.5 g, 14.1 mmol) in ACN (70 mL) was added TEA (1.43 g, 14.1 mmol) followed by dimethyl acetylenedicarboxylate (DMAD, 4.1 g, 28.2 mmol). The resulting crude reaction mixture was stirred at rt for 25 hrs after which TLC (Pet. ether: EtOAc 3/1) indicated the starting material was consumed with the formation of a less polar product. The reaction was concentrated and purified by silica gel chromatography (eluting with 0-30% EtOAc in Pet. ether) to afford P3 (3.0 g, 81%) as a colorless solid. ¹H NMR (400 MHZ, CHLOROFORM-d) δ 7.48 (d, J=7.9 Hz, 1H), 7.03 (d, J=5.3 Hz, 1H), 3.92 (s, 3H). 4-[(5-ethyl-1-{[2-(trimethylsilyl)ethoxy]methyl}-1H-pyrazol-3-yl)amino]-2-fluoro-5-methoxybenzonitrile (P4): Argon was bubbled through a yellow suspension of 4-bromo-2-fluoro-5-methoxybenzonitrile (P3) (2.0 g, 8.7 mmol), 5-ethyl-1-{[2-(trimethylsilyl)ethoxy]methyl}-1H-pyrazol-3-amine (2.5 g, 10.3 mmol, Intermediate O) and cesium carbonate (8.4 g, 25.8 mmol) in dioxane (40 mL) for 1 minute prior to the addition of Pd₂(dba)₃ (0.79 g, 0.86 mmol) and Xantphos (1.5 g, 2.6 mmol). The resulting mixture was heated at 80° C. for 18 hrs after which time LCMS showed the starting material was consumed. The reaction was cooled and diluted with EtOAc (200 mL), filtered, and concentrated to afford a brown gum, which was purified by silica gel chromatography (eluting with 0-10% EtOAc in Pet. ether) to afford P4 (3.5 g, 74%) as a yellow oil. LCMS m/z 391 [M+H]⁺.

N⁶-(5-ethyl-1-{[2-(trimethylsilyl)ethoxy]methyl}-1H-pyrazol-3-yl)-5-methoxy-1,2-benzoxazole-3,6-diamine (Intermediate P): A solution of acetohydroxamic acid (2.3 g, 30.1 mmol) and potassium t-butoxide (3.4 g, 30.1 mmol) in anhydrous DMF (60 mL) was stirred under nitrogen for 30 mins. To the resultant white mixture was added a solution of 4-[(5-ethyl-1-{[2-(trimethylsilyl)ethoxy]methyl}-1H-pyrazol-3-yl)amino]-2-fluoro-5-methoxybenzonitrile (P4) (3.9 g, 10.0 mmol) in anhydrous DMF (20 mL). After the addition, the crude reaction mixture was stirred at 80° C. for 16 hrs. LCMS indicated that ˜35% desired product was formed and starting material was still present. The reaction was cooled to rt before the addition of further acetohydroxamic acid (2.3 g, 30.1 mmol) and potassium t-butoxide (3.4 g, 30.1 mmol). After being purged with nitrogen, the reaction was stirred at 80° C. for 40 hrs. LCMS indicated that ˜ 55% desired product was formed and starting material was still present. The reaction was allowed to cool to rt before the addition of further acetohydroxamic acid (2.3 g, 30.1 mmol) and potassium t-butoxide (3.4 g, 30.1 mmol). After being purged with nitrogen, the reaction was stirred at 80° C. for 16 hrs and LCMS indicated that ˜80% desired product was formed. The reaction was stopped, cooled to rt, and concentrated in vacuo to remove DMF and afforded a residue that was dissolved in EtOAc (250 mL). This organic solution was washed with H₂O (250 mL) and the aqueous layer back-extracted with EtOAc (3×250 mL). The combined organic extracts were washed with saturated brine solution (250 mL), dried over sodium sulfate, filtered, and concentrated to afford a gum, which was purified by silica gel chromatography (eluting with 0-60% EtOAc in Pet. ether) to give Intermediate P as a solid with ca. 80% purity. The crude product was slurried in a mixture of DCM (5 mL) and Pet. ether (50 mL), stirred for 1 hr, and filtered to provide Intermediate P (1.2 g, 30%) as a white solid. LCMS m/z 404 [M+H]⁺; ¹H NMR (400 MHZ, CHLOROFORM-d) δ 7.88 (s, 1H), 6.96 (s, 1H), 6.78 (s, 1H), 5.91 (s, 1H), 5.35 (s, 2H), 3.95 (s, 3H), 3.67-3.63 (m, 2H), 2.72 (q, J=7.4 Hz, 2H), 1.31 (t, J=7.6 Hz, 3H), 0.95-0.87 (m, 2H), −0.01 (s, 9H). Note subjecting the recovered filtrates to silica gel chromatography (eluting with 0-60% EtOAc in Pet. ether) followed by a DCM/Pet. ether slurry (1:10) as described enabled a second batch of Intermediate P (1.6 g, 40%) as a yellow/orange solid.

### Method G

### Protocol for the Synthesis of Library Compounds

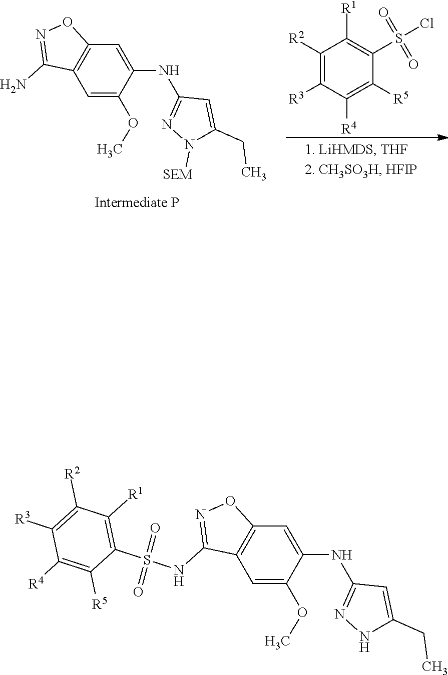

The reactions and stock solutions were set up in a glovebox environment. For the stock solutions, Intermediate P (170 mg) was dissolved in anhydrous THF (2.1 mL) leading to a clear solution. The stock solution of the base was prepared through the dissolution of solid LiHMDS (248 mg) in anhydrous THF (2.3 mL) to give a clear solution. The sulfonyl chloride monomers were purchased as required and supplied neat from our inventory in 1 mL glass tubes. To prepare the reactions, the solution of Intermediate P (100 μL corresponding to 20 μmol) was added to the sulfonyl chloride monomer (30 μmol) in a 1 mL glass vial followed by addition of the LiHMDS solution (100 μL, 60 μmol). The plate containing the array of glass vials was sealed, removed from the glove box, and shaken (950 rpm) at 60° C. for 16 hrs. LCMS analysis was carried out at this time to check for the presence of desired products, and the reactions were concentrated using an Evaporex N2. Saturated aq NaHCO₃ solution (200 μL) and EtOAc (300 μL) was added to each of the dried samples, which were then shaken at rt for 15 mins. The organic layer was separated, and the aqueous further extracted with EtOAc (2×200 μL). The combined organics were concentrated prior to addition of a 0.3 M solution of methanesulfonic acid in HFIP (200 μL, 60 μmol). The reactions were shaken at rt for 50 mins prior to concentration. The dried samples were reconstituted in DMSO (150 μL) and purified by mass-directed reverse-phase HPLC using a Sunfire C18 10×50 mm, 5 μm column (5 minute method: ACN/H₂O modified with 0.1% formic acid, 4 mL/minute).

TABLE 19

| Example | | | |
|---|---|---|---|
| Number | Structure/IUPAC Name | Analytical Data | Method |
| 124 | 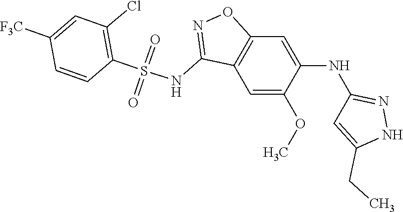 [SMILES: CCc1cc(Nc2cc3onc(NS(=O)(=O)c4ccc(C(F)(F)F)cc4Cl)c3cc2OC)n[nH]1] | LCMS m/z 516 [M + H]⁺ | G |
| | 2-chloro-N-{6-[(5-ethyl-1H- | | |
| | pyrazol-3-yl)amino]-5-methoxy- | | |
| | 1,2-benzoxazol-3-yl}-4- | | |
| | (trifluoromethyl)benzene-1- | | |
| | sulfonamide | | |
| 125 | 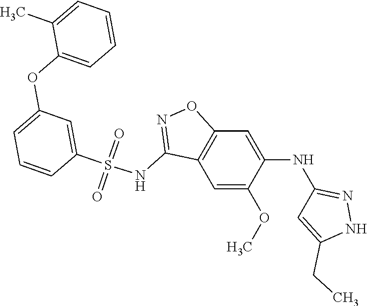 [SMILES: CCc1cc(Nc2cc3onc(NS(=O)(=O)c4cccc(Oc5ccccc5C)c4)c3cc2OC)n[nH]1] | LCMS m/z 520 [M + H]⁺ | G |
| | N-{6-[(5-ethyl-1 H-pyrazol-3- | | |
| | yl)amino]-5-methoxy-1,2- | | |
| | benzoxazol-3-yl}-3-(2- | | |
| | methylphenoxy)benzene-1- | | |
| | sulfonamide | | |
| 126 | 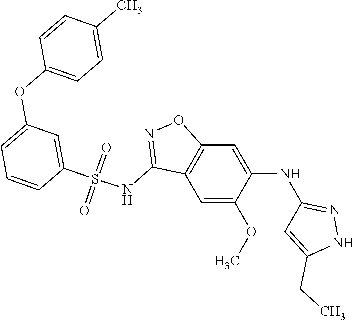 [SMILES: CCc1cc(Nc2cc3onc(NS(=O)(=O)c4cccc(Oc5ccc(C)cc5)c4)c3cc2OC)n[nH]1] | LCMS m/z 520 [M + H]⁺ | G |
| | N-{6-[(5-ethyl-1 H-pyrazol-3- | | |
| | yl)amino]-5-methoxy-1,2- | | |
| | benzoxazol-3-yl}-3-(4- | | |
| | methylphenoxy)benzene-1- | | |
| | sulfonamide | | |
| 127 | 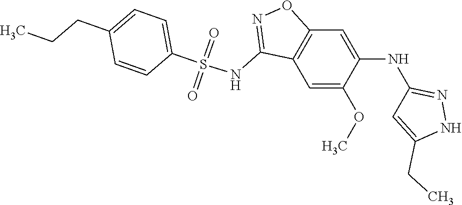 [SMILES: CCCc1ccc(S(=O)(=O)Nc2noc3cc(Nc4cc(CC)[nH]n4)c(OC)cc23)cc1] | LCMS m/z 456 [M + H]⁺ | G |
| | N-{6-[(5-ethyl-1 H-pyrazol-3- | | |
| | yl)amino]-5-methoxy-1,2- | | |
| | benzoxazol-3-yl}-4- | | |
| | propylbenzene-1-sulfonamide | | |
| 128 | 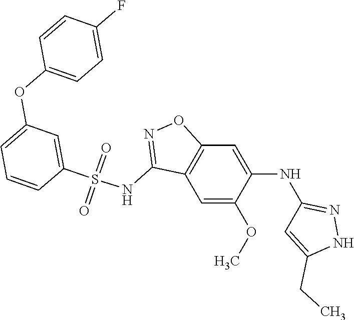 [SMILES: CCc1cc(Nc2cc3onc(NS(=O)(=O)c4cccc(Oc5ccc(F)cc5)c4)c3cc2OC)n[nH]1] | LCMS m/z 524 [M + H]⁺ | G |
| | N-{6-[(5-ethyl-1 H-pyrazol-3- | | |
| | yl)amino]-5-methoxy-1,2- | | |
| | benzoxazol-3-yl}-3-(4- | | |
| | fluorophenoxy)benzene-1- | | |
| | sulfonamide | | |
| 129 | 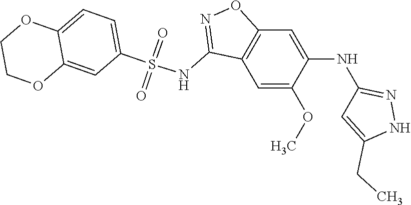 [SMILES: CCc1cc(Nc2cc3onc(NS(=O)(=O)c4ccc5c(c4)OCCO5)c3cc2OC)n[nH]1] | LCMS m/z 472 [M + H]⁺ | G |
| | N-{6-[(5-ethyl-1 H-pyrazol-3- | | |
| | yl)amino]-5-methoxy-1,2- | | |
| | benzoxazol-3-yl}-2,3-dihydro- | | |
| | 1,4-benzodioxine-6- | | |
| | sulfonamide | | |
| 130 | 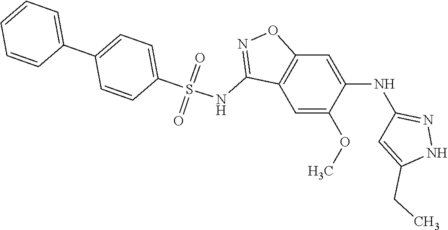 [SMILES: CCc1cc(Nc2cc3onc(NS(=O)(=O)c4ccc(-c5ccccc5)cc4)c3cc2OC)n[nH]1] | LCMS m/z 490 [M + H]⁺ | G |
| | N-{6-[(5-ethyl-1 H-pyrazol-3- | | |
| | yl)amino]-5-methoxy-1,2- | | |
| | benzoxazol-3-yl}[1,1′-biphenyl]- | | |
| | 4-sulfonamide | | |
| 131 | 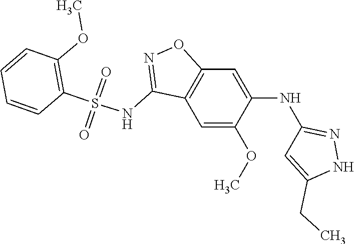 [SMILES: CCc1cc(Nc2cc3onc(NS(=O)(=O)c4ccccc4OC)c3cc2OC)n[nH]1] | LCMS m/z 443 [M + H]⁺ | G |
| | N-{6-[(5-ethyl-1 H-pyrazol-3- | | |
| | yl)amino]-5-methoxy-1,2- | | |
| | benzoxazol-3-yl}-2- | | |
| | methoxybenzene-1- | | |
| | sulfonamide | | |
| 132 | 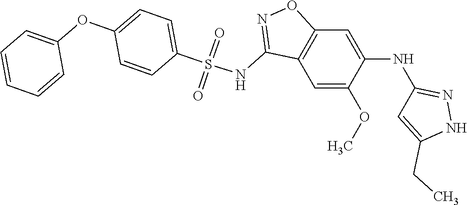 [SMILES: CCc1cc(Nc2cc3onc(NS(=O)(=O)c4ccc(Oc5ccccc5)cc4)c3cc2OC)n[nH]1] | LCMS m/z 506 [M + H]⁺ | G |
| | N-{6-[(5-ethyl-1 H-pyrazol-3- | | |
| | yl)amino]-5-methoxy-1,2- | | |
| | benzoxazol-3-yl}-4- | | |
| | phenoxybenzene-1- | | |
| | sulfonamide | | |
| 133 | 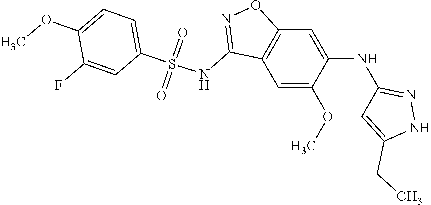 [SMILES: CCc1cc(Nc2cc3onc(NS(=O)(=O)c4ccc(OC)c(F)c4)c3cc2OC)n[nH]1] | LCMS m/z 462 [M + H]⁺ | G |
| | N-{6-[(5-ethyl-1 H-pyrazol-3- | | |
| | yl)amino]-5-methoxy-1,2- | | |
| | benzoxazol-3-yl}-3-fluoro-4- | | |
| | methoxybenzene-1- | | |
| | sulfonamide | | |
| 134 | 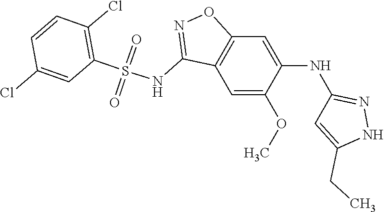 [SMILES: CCc1cc(Nc2cc3onc(NS(=O)(=O)c4cc(Cl)ccc4Cl)c3cc2OC)n[nH]1] | LCMS m/z 482 [M + H]⁺ | G |
| | 2,5-dichloro-N-{6-[(5-ethyl-1 H- | | |
| | pyrazol-3-yl)amino]-5-methoxy- | | |
| | 1,2-benzoxazol-3-yl}benzene- | | |
| | 1-sulfonamide | | |
| 135 | 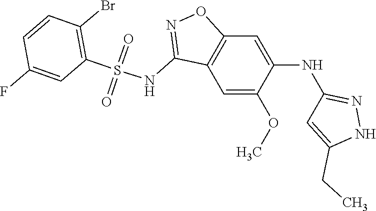 [SMILES: CCc1cc(Nc2cc3onc(NS(=O)(=O)c4cc(F)ccc4Br)c3cc2OC)n[nH]1] | LCMS m/z 510/512 [M + H]⁺ | G |
| | 2-bromo-N-{6-[(5-ethyl-1H- | | |
| | pyrazol-3-yl)amino]-5-methoxy- | | |
| | 1,2-benzoxazol-3-yl}-5- | | |
| | fluorobenzene-1-sulfonamide | | |
| 136 | 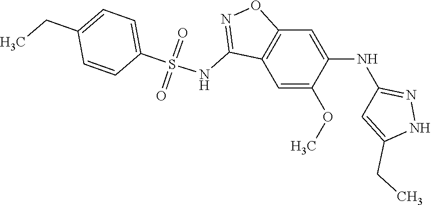 [SMILES: CCc1ccc(S(=O)(=O)Nc2noc3cc(Nc4cc(CC)[nH]n4)c(OC)cc23)cc1] | LCMS m/z 442 [M + H]⁺ | G |
| | 4-ethyl-N-{6-[(5-ethyl-1 H- | | |
| | pyrazol-3-yl)amino]-5-methoxy- | | |
| | 1,2-benzoxazol-3-yl}benzene- | | |
| | 1-sulfonamide | | |
| 137 | 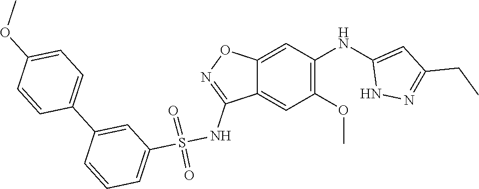 [SMILES: CCc1cc(Nc2cc3onc(NS(=O)(=O)c4cccc(-c5ccc(OC)cc5)c4)c3cc2OC)[nH]n1] | LCMS m/z 520.4 [M + H]⁺ | G |
| | N-{6-[(3-ethyl-1 H-pyrazol-5- | | |
| | yl)amino]-5-methoxy-1,2- | | |
| | benzoxazol-3-yl}-4′- | | |
| | methoxy[1,1′-biphenyl]-3- | | |
| | sulfonamide | | |

### Example 138:4-(16-[(3-Ethyl-1H-pyrazol-5-yl)amino]-5-methoxy-1,2-benzoxazol-3-yl}sulfamoyl)-3-methoxy-N-15-14-(6-methyl-1,2,4,5-tetrazin-3-yl)benzamido]pentyl}benzamide

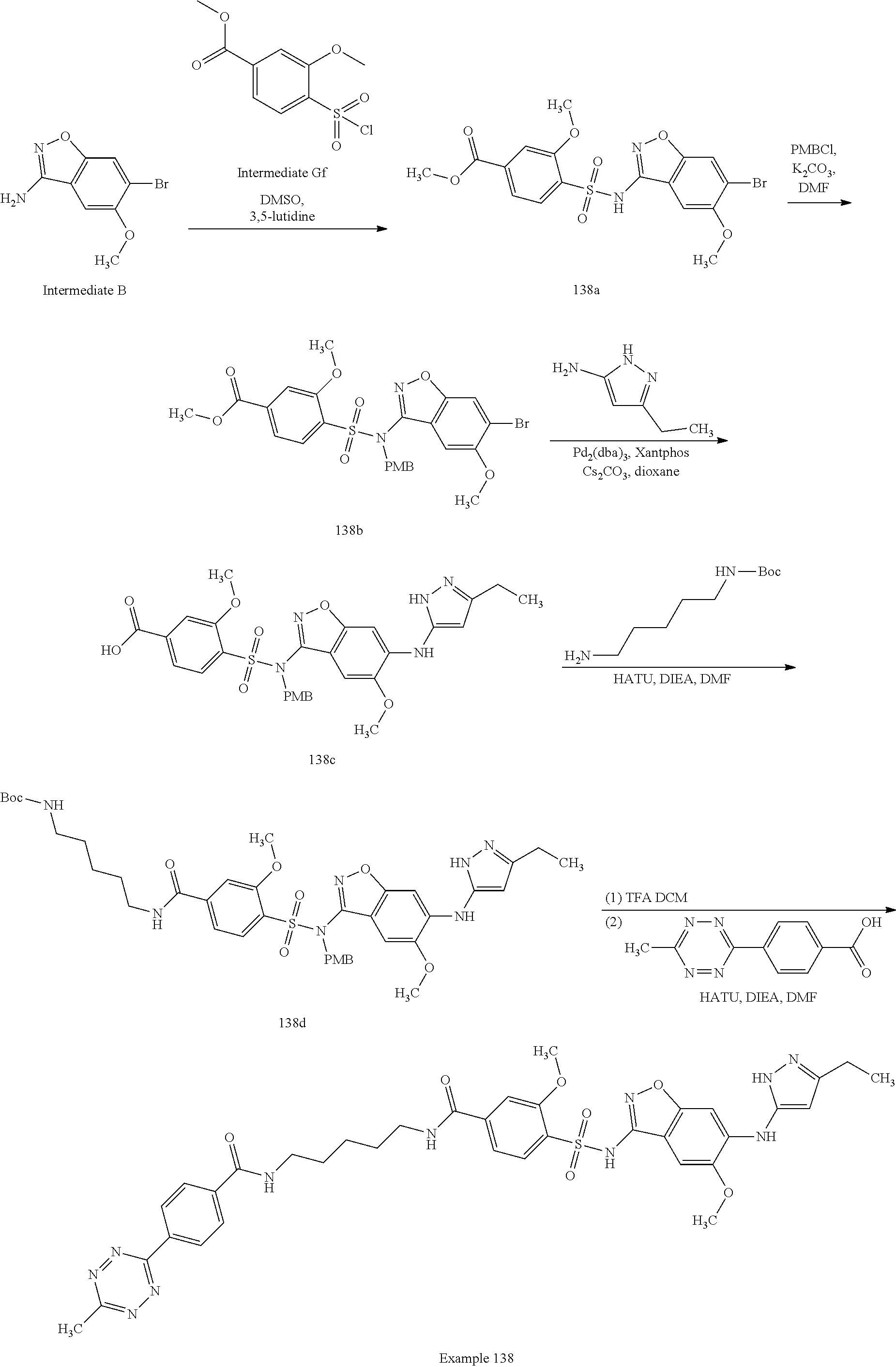

### Methyl 4-(chlorosulfonyl)-3-methoxybenzoate (Intermediate Gf)

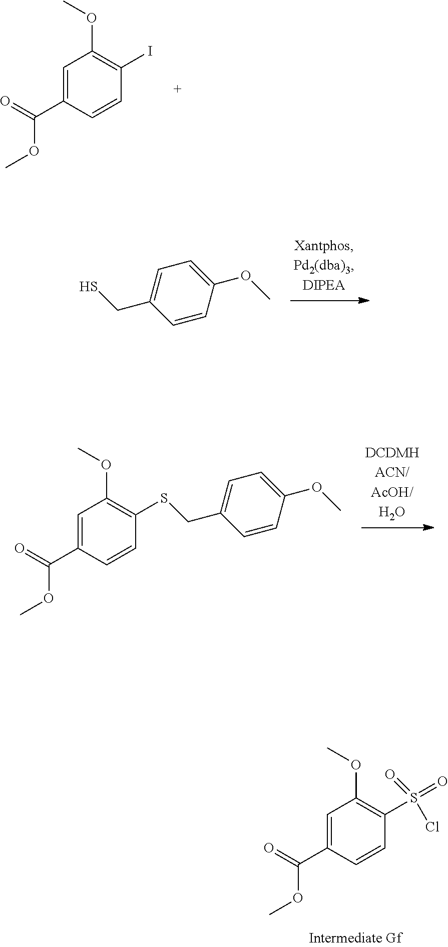

A mixture of methyl 4-iodo-3-methoxybenzoate (3500 mg, 11.98 mmol), 4-methoxybenzyl mercaptan (2030 mg, 13.2 mmol), DIPEA (3100 mg, 24.0 mmol), xantphos (693 mg, 1.20 mmol) and Pd₂(dba)₃ (549 mg, 0.599 mmol) in anhydrous dioxane (50.0 mL) was degassed with nitrogen 6 times and then stirred at 110° C. for 15 hrs. After cooling, the reaction was filtered and the filtrate was concentrated under reduced pressure. The resulting crude was purified by flash silica gel column chromatography (eluting with 0-5% EtOAc in Pet ether) to give methyl 3-methoxy-4-{[(4-methoxyphenyl)methyl]sulfanyl]benzoate (3500 mg, 91.7%) as a yellow solid, ¹H NMR (400 MHZ, CHLOROFORM-d) δ 7.60 (dd, J=1.38, 8.00 Hz, 1H), 7.49 (d, J=1.00 Hz, 1H), 7.31 (d, J=8.50 Hz, 2H), 7.24 (d, J=8.00 Hz, 1H), 6.86 (d, J=8.63 Hz, 2H), 4.15 (s, 2H), 3.96 (s, 3H), 3.93 (s, 3H), 3.74-3.86 (m, 3H)

To a stirred solution of methyl 3-methoxy-4-{[(4-methoxyphenyl)methyl]sulfanyl}benzoate (1000.0 mg, 3.141 mmol) in a mixture of ACN-HOAc-water (35 mL-1.8 mL-1.3 mL) was added 1,3-dichloro-5,5-dimethylhydantoin (1240 mg, 6.28 mmol) in portions at 0° C. After addition, the reaction mixture was stirred at 0-10° C. for 1 hr and was then diluted with EtOAc (20 mL). The organic layer was washed with aqueous NaHCO₃ (5 mL), brine (5 mL), dried over Na₂SO₄ and concentrated under reduced pressure. The resulting crude was purified by flash silica gel column chromatography (eluting with 0-40% EtOAc in Pet ether) to give Intermediate Gf (500 mg, 60.1%) as white solid. ¹H NMR (400 MHZ, CHLOROFORM-d) δ 8.06 (d, J=8.28 Hz, 1H), 7.80 (d, J=1.00 Hz, 1H), 7.76 (dd, J=1.51, 8.28 Hz, 1H), 4.12-4.18 (m, 3H), 3.98-4.04 (m, 3H)

### Methyl 4-[(6-bromo-5-methoxy-1,2-benzoxazol-3-yl)sulfamoyl]-3-methoxybenzoate (138a)

A solution of Intermediate B (400.0 mg, 1.65 mmol), Intermediate Gf (500.0 mg, 1.89 mmol), 3,5-lutidine (0.563 mL, 4.94 mmol) and DMSO (1.65 mL, 0.0823 mmol) in ACN (4 mL) was stirred at 32° C. for 16 hrs. LCMS showed the main peak was the desired product and that 40% of starting material remained. 3,5-lutidine (0.563 mL, 4.94 mmol) was added and the reaction was stirred at 32° C. for a further 16 hrs. The reaction was diluted with EtOAc and the organic layer was washed with 1N citric acid, dried over sodium sulfate, filtered and concentrated under reduced pressure. The resulting residue was purified by flash silica gel column chromatography (eluting with 0-100% ethyl acetate in DCM) to give 138a (250 mg, 32.2%) as yellow gum, LCMS m/z 471.0 [M+H]+.

### Methyl 4-{(6-bromo-5-methoxy-1,2-benzoxazol-3-yl) [(4-methoxyphenyl)methyl]sulfamoyl}-3-methoxybenzoate (138b)

A solution of 138a (250 mg, 0.530 mmol), 4-methoxybenzyl chloride (91.4 mg, 0.584 mmol) and K₂CO₃ (88.0 mg, 0.637 mmol) in DMF (5.0 mL) was stirred at ˜80° C. for ˜16 hrs. The reaction was cooled to 15° C., poured into H₂O (100 mL) and stirred for 20 mins. Some white solid precipitated. EtOAc was added and the aqueous layer was extracted three times with EtOAc. The combined organic layer was dried over sodium sulfate, filtered and concentrated under reduced pressure. The resulting crude was purified by flash silica gel column chromatography (eluting with 0-30% EtOAc in Pet ether) to give 138b (220 mg, 70.1%) as a gum, LCMS m/z 614.8 [M+H+Na]⁺.

4-({6-[(5-Ethyl-1H-pyrazol-3-yl)amino]-5-methoxy-1,2-benzoxazol-3-yl}[(4-methoxyphenyl)methyl]sulfamoyl)-3-methoxybenzoic acid (138c): Prepared from methyl 4-{(6-bromo-5-methoxy-1,2-benzoxazol-3-yl)[(4-methoxyphenyl)methyl]sulfamoyl}-3-methoxybenzoate 138b and 4-ethyl-1H-pyrazole using method B to give 138c (100 mg, 44%) as a yellow solid. LCMS m/z 608 [M+H]⁺.

tert-Butyl {5-[4-({6-[(5-ethyl-1H-pyrazol-3-yl)amino]-5-methoxy-1,2-benzoxazol-3-yl}[(4-methoxyphenyl)methyl]sulfamoyl)-3-methoxybenzamido]pentyl}carbamate (138d): A solution of 4-({6-[(5-ethyl-1H-pyrazol-3-yl)amino]-5-methoxy-1,2-benzoxazol-3-yl}[(4-methoxyphenyl)methyl]sulfamoyl)-3-methoxybenzoic acid (138c) (100 mg, 0.165 mmol), Boc-1,5-diaminopentane (50 mg, 0.247 mmol), HATU (94 mg, 0.247 mmol) and N,N-diisopropylethylamine (64 mg, 0.494 mmol) in dimethylformamide (2 mL) was stirred for 16 hrs, then quenched with saturated aq sodium bicarbonate, and partitioned between EtOAc and H₂O. The H₂O layer was extracted three more times with EtOAc. The combined organic layers were washed with brine, dried over sodium sulfate, filtered, concentrated, and purified by silica gel chromatography (eluting with 0-10% methanol in DCM) to give 138d (120 mg, 92%) as a brown oil. LCMS m/z 792 [M+H]⁺.

4-({6-[(3-Ethyl-1H-pyrazol-5-yl)amino]-5-methoxy-1,2-benzoxazol-3-yl}sulfamoyl)-3-methoxy-N-{5-[4-(6-methyl-1,2,4,5-tetrazin-3-yl)benzamido]pentyl}benzamide (Example 138): tert-Butyl {5-[4-({6-[(5-ethyl-1H-pyrazol-3-yl)amino]-5-methoxy-1,2-benzoxazol-3-yl}[(4-methoxyphenyl)methyl]sulfamoyl)-3-methoxybenzamido]pentyl}carbamate (138d) (120 mg, 0.152 mmol) was stirred in DCM (4 mL) and trifluoracetic acid (4 mL) for 16 hrs and concentrated. The residue was stirred in dimethylformamide (2 mL) and 4-(6-methyl-1,2,4,5-tetrazin-3-yl)benzoic acid (30 mg, 0.14 mmol), HATU (79.1 mg, 0.208 mmol) and N,N-diisopropylethylamine (143 mg, 1.11 mmol) were added. The mixture was stirred for 16 hrs, concentrated and purified by prep HPLC on Phenomenex C18 (75×30 mm, 3 micron) column eluting with 35-75% ACN-H₂O over 9 mins to give Example 138 (12 mg, 11%) as a solid. LCMS m/z 770 [M+H]⁺; ¹H NMR (400 MHZ, DMSO-d₆) δ 11.33 (s, 1H), 8.66-8.54 (m, 2H), 8.46 (d, J=8.4 Hz, 2H), 8.07 (s, 2H), 8.01 (d, J=8.5 Hz, 2H), 7.84 (d, J=8.1 Hz, 1H), 7.49-7.40 (m, 2H), 7.27 (s, 1H), 5.81 (s, 1H), 3.82 (d, J=4.5 Hz, 6H), 3.23-3.14 (m, 4H), 2.95 (s, 3H), 2.53-2.47 (m, 2H), 1.59-1.45 (m, 4H), 1.38-1.25 (m, 2H), 1.10 (t, J=1.0 Hz, 3H).

### Example 139:2-Ethoxy-5-fluoro-N-{6-[(4-fluoro-1H-pyrazol-3-yl)amino]-5-methoxy-1,2-benzoxazol-3-yl}-4-methylbenzene-1-sulfonamide

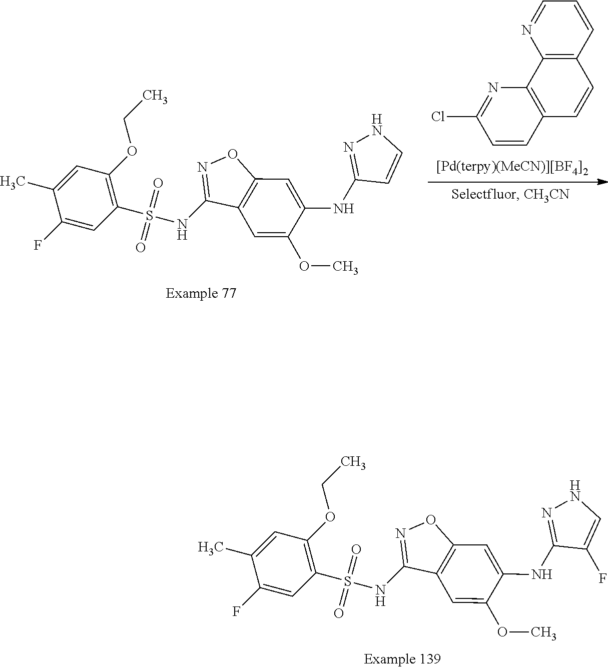 [SMILES: CCOc1cc(C)c(F)cc1S(=O)(=O)Nc1noc2cc(Nc3cc[nH]n3)c(OC)cc12 ; Clc1ccc2ccc3cccnc3c2n1 ; CCOc1cc(C)c(F)cc1S(=O)(=O)Nc1noc2cc(Nc3n[nH]cc3F)c(OC)cc12]

A catalyst complex solution was made as follows. To a separate 1 dram vial was made a bulk stock solution of 2-chloro-1,10-phenanthroline (0.21 mg), and [Pd(terpy)(ACN)][BF₄]₂ (0.51 mg) dissolved in ACN 91 μL, which resulted in a yellow solution. To a 1 dram pressure release vial containing 2-ethoxy-5-fluoro-N-{5-methoxy-6-[(1H-pyrazol-3-yl)amino]-1,2-benzoxazol-3-yl}-4-methylbenzene-1-sulfonamide (Example 77) (1.3 mg, 2.82 μmol), was added Selectfluor® (2.0 mg, 5.6 μmol) and ACN (94 μL), followed by the catalyst complex solution (14 μL, consisting of palladium complex (0.078 mg, 0.14 μmol, 5 mol %) and 2-chloro-phenanthroline (0.03 mg, 0.14 μmol, 5 mol %). The crude reaction mixture (noted as an orange solution) was stirred at 25° C. overnight and purified by HPLC to provide 0.03 mg of Example 139. LCMS m/z 480 [M+H]⁺; ¹H NMR (600 MHZ, DMSO-d₆) δ 12.40 (s, 1H), 11.38 (s, 1H), 7.88 (dd, J=4.7, 2.0 Hz, 1H), 7.62 (s, 1H), 7.56 (d, J=8.9 Hz, 1H), 7.45 (s, 1H), 7.31 (s, 1H), 7.15 (d, J=5.9 Hz, 1H), 4.10 (q, J=7.0 Hz, 2H), 3.90 (s, 3H), 2.27 (s, 3H), 1.22 (t, J=7.0 Hz, 4H).

### Examples 140 and 141: N-{6-[(3-cyclopropyl-1H-pyrazol-5-yl)amino]-5-methoxy-1,2-benzoxazol-3-yl}-2,6-dimethoxy-4-(oxolan-2-yl)benzene-1-sulfonamide

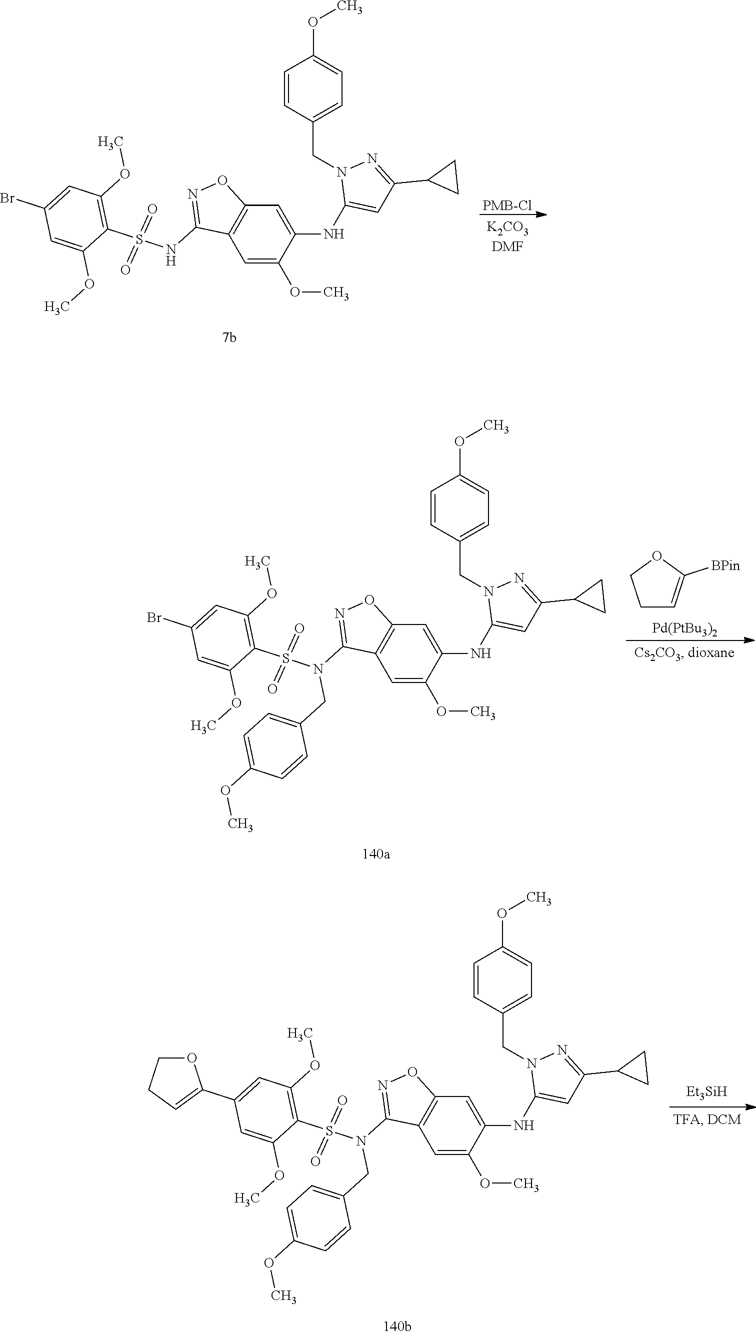

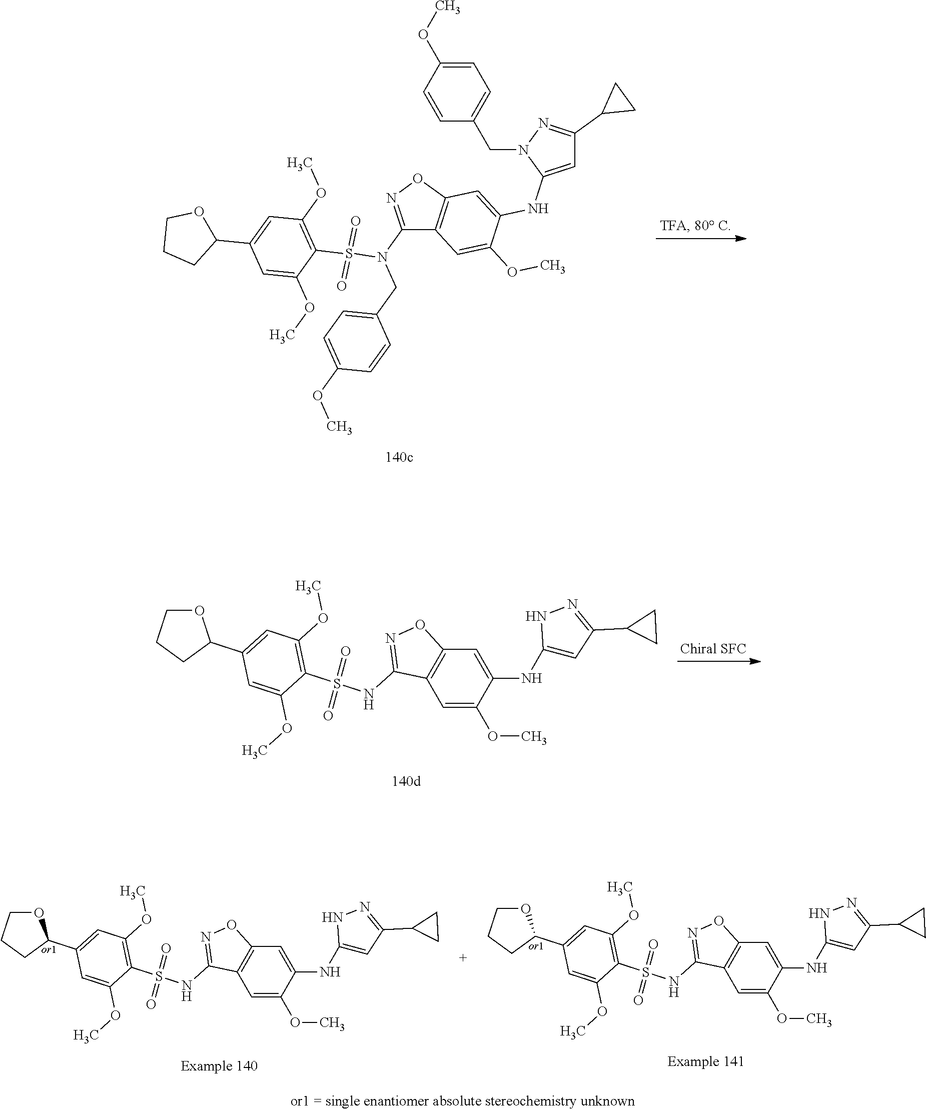 [SMILES: COc1ccc(CN(c2noc3cc(Nc4cc(C5CC5)nn4Cc4ccc(OC)cc4)c(OC)cc23)S(=O)(=O)c2c(OC)cc(C3CCCO3)cc2OC)cc1 ; COc1cc2c(NS(=O)(=O)c3c(OC)cc(C4CCCO4)cc3OC)noc2cc1Nc1cc(C2CC2)n[nH]1 ; COc1cc2c(NS(=O)(=O)c3c(OC)cc([C@H]4CCCO4)cc3OC)noc2cc1Nc1cc(C2CC2)n[nH]1 ; COc1cc2c(NS(=O)(=O)c3c(OC)cc([C@@H]4CCCO4)cc3OC)noc2cc1Nc1cc(C2CC2)n[nH]1]

### Step 1: Synthesis of 4-bromo-N-[6-({3-cyclopropyl-1-[(4-methoxyphenyl)methyl]-1H pyrazol-5-yl}amino)-5-methoxy-1,2-benzoxazol-3-yl]-2,6-dimethoxy-N-[(4-methoxyphenyl)methyl]benzene-1-sulfonamide (140a)

To a solution of 4-bromo-N-[6-({3-cyclopropyl-1-[(4-methoxyphenyl)methyl]-1H-pyrazol-5-yl}amino)-5-methoxy-1,2-benzoxazol-3-yl]-2,6-dimethoxybenzene-1-sulfonamide (7b) and K₂CO₃ (491 mg, 3.55 mmol) in DMF (11.8 mL) was added para-methoxybenzyl chloride (185 mg, 1.18 mmol). The mixture was stirred at 80° C. for 3.5 hrs. The crude reaction mixture was filtered and the filtrate concentrated under reduced pressure. The crude product was purified over silica gel and eluted with 0-40% EtOAc-Pet. ether, which provided 140a as a yellow solid (680 mg, 71% yield). LCMS m/z 805 (M+H).

### Step 2: Synthesis of N-[6-({3-cyclopropyl-1-[(4-methoxyphenyl)methyl]-1H-pyrazol-5-yl}amino)-5-methoxy-1,2-benzoxazol-3-yl]-4-(4,5-dihydrofuran-2-yl)-2,6-dimethoxy-N-[(4-methoxyphenyl)methyl]benzene-1-sulfonamide (140b)

A mixture of 4-bromo-N-[6-({3-cyclopropyl-1-[(4-methoxyphenyl)methyl]-1H-pyrazol-5-yl}amino)-5-methoxy-1,2-benzoxazol-3-yl]-2,6-dimethoxy-N-[(4-methoxyphenyl)methyl]benzene-1-sulfonamide (140a) (580 mg, 0.72 mmol), 2-(4,5-dihydrofuran-2-yl)-4,4,5,5-tetramethyl-1,3,2-dioxaborolane (250 mg, 1.28 mmol) bis(tri-tert-butylphosphine) palladium (37 mg, 0.07 mmol), Cs₂CO₃ (705 mg, 2.16 mmol) was stirred at 80° C. for one hr. The crude reaction mixture was combined with the crude reaction mixture from three other reactions (247 mg combined theoretical yield for these other three reactions) and the solvent removed under reduced pressure. The crude product was purified over silica gel (0-45% EtOAc-Pet. ether) and provided 400 mg of 140b as a white solid (55% pure by LCMS). The white solid was further purified by SFC (60% ethanol with 0.1% ammonium hydroxide in CO₂; 80 mL/min flow rate; Daicel Chiralpak® AD 250×30 mm, 10 μm) and provided 50 mg of 140b as a colorless oil (6% yield).

### Step 3: Synthesis of N-[6-({3-cyclopropyl-1-[(4-methoxyphenyl)methyl]-1H-pyrazol-5-yl}amino)-5-methoxy-1,2-benzoxazol-3-yl]-2,6-dimethoxy-N-[(4-methoxyphenyl)methyl]-4-(oxolan-2-yl)benzene-1-sulfonamide (140c)

To a solution of N-[6-({3-cyclopropyl-1-[(4-methoxyphenyl)methyl]-1H-pyrazol-5-yl}amino)-5-methoxy-1,2-benzoxazol-3-yl]-4-(4,5-dihydrofuran-2-yl)-2,6-dimethoxy-N-[(4-methoxyphenyl)methyl]benzene-1-sulfonamide (140b) (50 mg, 0.06 mmol) in TFA (1.0 mL) and DCM (1.0 mL) was added triethylsilane (0.4 mL, 2.5 mmol). The crude reaction mixture was stirred for 30 mins and the solvent was removed under reduced pressure and provided 50 mg of 140c as a yellow oil.

### Step 4: Synthesis of N-{6-[(3-cyclopropyl-1H-pyrazol-5-yl)amino]-5-methoxy-1,2-benzoxazol-3-yl}-2,6-dimethoxy-4-(oxolan-2-yl)benzene-1-sulfonamide (140d)

A solution of N-[6-({3-cyclopropyl-1-[(4-methoxyphenyl)methyl]-1H-pyrazol-5-yl}amino)-5-methoxy-1,2-benzoxazol-3-yl]-2,6-dimethoxy-N-[(4-methoxyphenyl)methyl]-4-(oxolan-2-yl)benzene-1-sulfonamide (140c) (350 mg, 0.6 mmol) in TFA (3 mL) was heated at 80° C. for 16 hrs. The crude reaction mixture was concentrated under reduced pressure and purified over silica gel (12 g, 0-60% EtOAc-Pet. ether) which provided 80 mg of 140d as a yellow solid (50% pure by LCMS). The yellow solid was combined with 20 mg of a crude product from another experiment (36 mg theoretical yield) and purified by SFC (35% ethanol with 0.1% ammonium hydroxide in CO₂; 80 mL/min flow rate; Daicel Chiralcel OJ 250×30 mm, 10 μm). Two isomers were isolated (10 mg each of a white solid).

### N-{6-[(3-cyclopropyl-1H-pyrazol-5-yl)amino]-5-methoxy-1,2-benzoxazol-3-yl}-2,6-dimethoxy-4-(oxolan-2-yl)benzene-1-sulfonamide (Example 140)

The first eluting peak was further purified using SFC (50% ethanol with 0.1% ammonium hydroxide in CO₂; 80 mL/min flow rate; Daicel Chiralcel OD 250×30 mm, 10 μm) and gave Example 140 as a white solid (6.3 mg, 2% yield). ¹H NMR (400 MHZ, CHLOROFORM-d) δ 7.63 (s, 1H), 7.47 (s, 1H), 7.16 (br.s, 1H), 6.57 (s, 2H), 5.75 (br. s, 1H), 4.83 (t, J=7.2 Hz, 1H), 4.03-3.93 (m, 1H), 3.96-3.94 (m, 4H), 3.91 (s, 6H), 2.26 (br d, J=6.1 Hz, 1H), 1.89 (br d, J=6.9 Hz, 2H), 1.82-1.76 (m, 1H), 1.69-1.62 (m, 2H), 0.98-0.88 (m, 2H), 0.72-0.66 (m, 2H); LCMS m/z 556.2 (M+H)⁺; [a]_{D}²⁶=−19.3° (C. 0.1, CH₃OH). N-{6-[(3-cyclopropyl-1H-pyrazol-5-yl)amino]-5-methoxy-1,2-benzoxazol-3-yl}-2,6-dimethoxy-4-(oxolan-2-yl)benzene-1-sulfonamide (Example 141)

The second eluting peak was further purified using SFC (50% ethanol with 0.1% ammonium hydroxide in CO₂; 150 mL/min flow rate; Daicel Chiralcel AD 250×30 mm, 10 μm) and gave Example 141 as a white solid (6.0 mg, 2% yield). ¹H NMR (400 MHZ, CHLOROFORM-d) δ 7.66 (s, 1H), 7.47 (s, 1H), 7.11 (br.s, 1H), 6.57 (s, 2H), 5.73 (br. s, 1H), 4.83 (t, J=7.2 Hz, 1H), 4.01-3.94 (m, 1H), 3.98-3.94 (m, 4H), 3.91 (s, 6H), 2.37-2.30 (m, 1H), 2.02-1.94 (m, 1H), 1.90-1.84 (m, 1H), 1.76-1.63 (m, 2H), 0.96-0.91 (m, 2H), 0.72-0.64 (m, 2H); LCMS m/z 556.3 (M+H)⁺; [a]_{D}²⁶=+4.0° (C. 0.1, CH₃OH).

### Example 140: Procedure to determine absolute stereochemistry N-16-1 (3-cyclopropyl-1H-pyrazol-5-yl)amino]-5-methoxy-1,2-benzoxazol-3-yl}-2,6-dimethoxy-4-[(2S)-oxolan-2-yl]benzene-1-sulfonamide

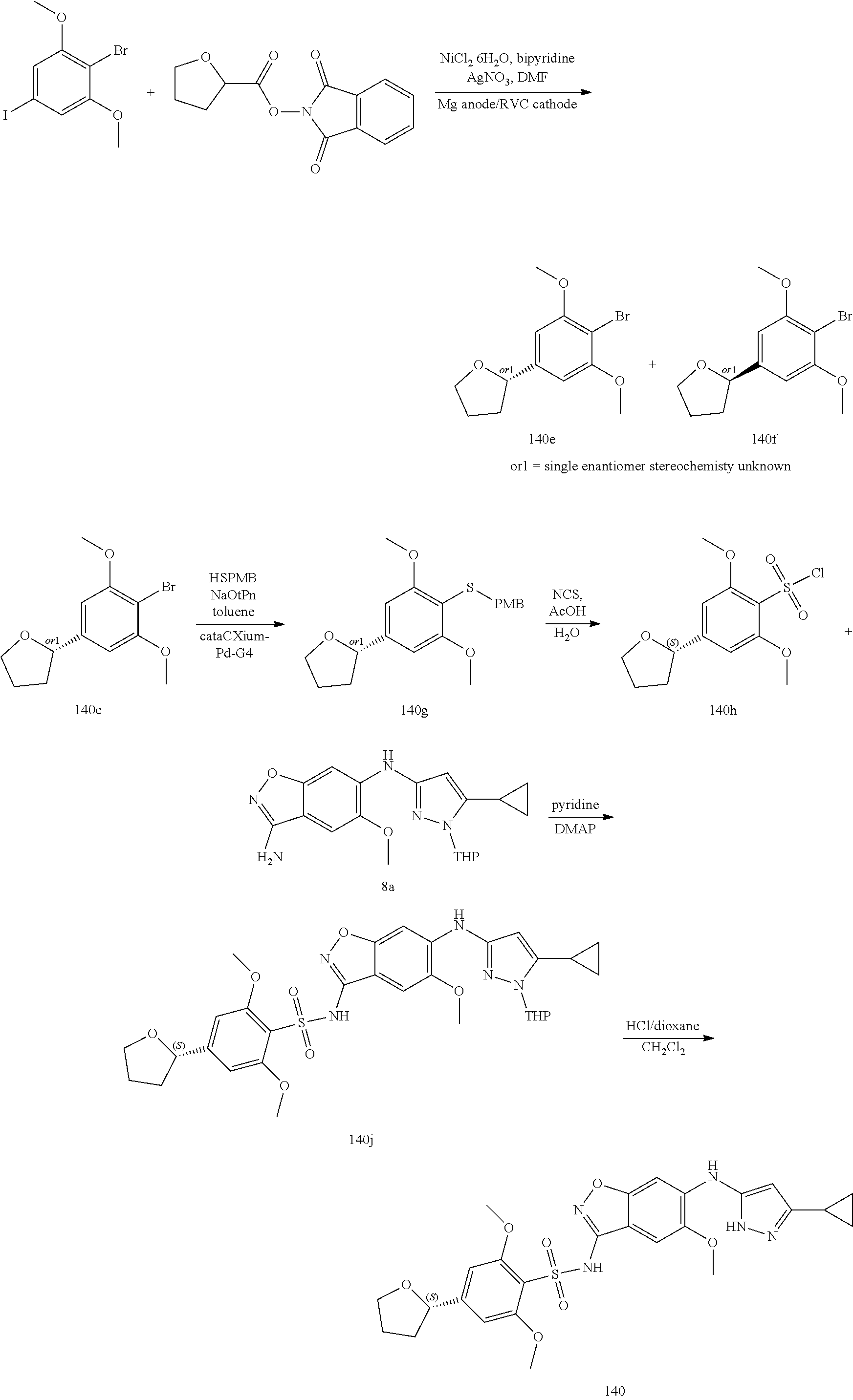

### Step 1: Synthesis of 2-(4-bromo-3,5-dimethoxyphenyl)oxolane (140e) and 2-(4-bromo-3,5-dimethoxyphenyl)oxolane (140f)

Reaction was run in four batches. A vial was charged with 2-bromo-5-iodo-1,3-dimethoxybenzene (0.89 g, 2.6 mmol), 2-[(oxolane-2-carbonyl)oxy]-1H-isoindole-1,3 (2H)-dione (0.52 g, 2.0 mmol), nickel chloride hexahydrate (95 mg, 0.4 mmol), 2,2′-bipyridine (62 mg, 0.4 mmol), DMF (15.0 mL) and a stir bar. The mixture was stirred for about 5 mins, then silver nitrate (170 mg, 1.0 mmol) was added. The vial was closed with an IKA ElectraSyn 2.0 vial cap with a magnesium sacrificial anode (left side) and a reticulated vitreous carbon (RVC) cathode (right side), then the vial was immediately placed on an IKA ElectraSyn 2.0 stir plate. Electrolysis was set to 40 mA, 2.0 mmol, 4.0 F/mol. The mixture was allowed to stand for approximately 24 hrs. The resulting crude reaction mixtures from four batches were combined then added to 600 mL sat. aq. NaHCO₃ and extracted with MTBE (3×200 mL). The combined organic extracts were washed with water (100 mL) and brine (100 mL), then dried over Na₂SO₄ and filtered thru a plug of silica gel (washed with 200 mL of EtOAc) and concentrated to give a beige semi-solid. To the solid was added 10 mL of DCM. The mixture was filtered and rinsed with DCM (2×10 mL). The resulting amber filtrate was concentrated and purified by column chromatography (SiO₂, eluting with 5-30% EtOAc in heptane) to give the racemic 2-(4-bromo-3,5-dimethoxyphenyl)oxolane (1.3 g, 56%) as an off-white solid. The enantiomers were separated by chiral SFC (14% methanol in CO₂; 100 mL/min flow rate, 120 bar; Regis (R,R) Whelk-O1 250×21.1 mm, 10 μm).

### 2-(4-bromo-3,5-dimethoxyphenyl)oxolane (140e)

The first eluting peak was isolated as a white solid (575 mg, 44%). ¹H NMR (400 MHZ, DMSO-d₆) δ 6.67 (s, 2H), 4.80 (t, J=7.2 Hz, 1H), 4.05-3.99 (m, 1H), 3.82-3.79 (m, 7H), 2.36-2.28 (m, 1H), 1.99-1.89 (m, 2H), 1.74-1.63 (m, 1H).; [a]_{D}²²=−30.1° (c 0.2, MeOH).

### 2-(4-bromo-3,5-dimethoxyphenyl)oxolane (140f)

The second eluting peak was isolated as a white solid (565 mg, 43%). ¹H NMR (400 MHZ, DMSO-d₆) δ 6.67 (s, 2H), 4.80 (t, J=7.2 Hz, 1H), 4.06-3.98 (m, 1H), 3.85-3.79 (m, 7H), 2.36-2.28 (m, 1H), 1.97-1.90 (m, 2H), 1.74-1.64 (m, 1H).; [a]_{D}²²=+21.9° (c 0.2, MeOH).

### Step 2: Synthesis of 2-(3,5-dimethoxy-4-{[(4-methoxyphenyl)methyl]sulfanyl}phenyl)oxolane (140 g)

A vial was charged with 2-(4-bromo-3,5-dimethoxyphenyl)oxolane (140e) (575 mg, 2.0 mmol), CataCXium-Pd-G4 (149 mg, 0.2 mmol), 4-methoxybenzyl mercaptan (371 mg, 2.4 mmol), sodium 2-methylbutan-2-olate (1.65 g, 1.8 mL, 40% wt, 6.01 mmol) in toluene (20 mL). The vial was sealed, flushed three times with nitrogen. The reaction was heated at 100° C. for 18 hrs to give a light brown/orange solution. Sat. aq. NaHCO₃ (20 mL) was added to the stirred reaction mixture, which was extracted with EtOAc (3×20 mL). The combined organic extracts were washed with brine (100 mL), then dried over Na₂SO₄, filtered, and concentrated to give a yellow oil that was purified by column chromatography (SiO₂, eluting with 5-40% EtOAc in heptane) to give compound 140 g (526 mg, 73%) as a thick orange oil. ¹H NMR (400 MHZ, DMSO-d₆) δ 7.12 (d, J=8.56 Hz, 2H) 6.79 (d, J=8.56 Hz, 2H) 6.58 (s, 2H) 4.77 (t, J=7.21 Hz, 1H) 4.05-3.97 (m, 1H) 3.90 (s, 2H) 3.85-3.79 (m, 1H) 3.77 (s, 6H) 3.70 (s, 3H) 2.36-2.22 (m, 1H) 1.97-1.88 (m, 2H) 1.73-1.61 (m, 1H).

### Step 3: Synthesis of 2,6-dimethoxy-4-[(2S)-oxolan-2-yl]benzene-1-sulfonyl chloride (140h) small molecule x-ray for stereochemical determination

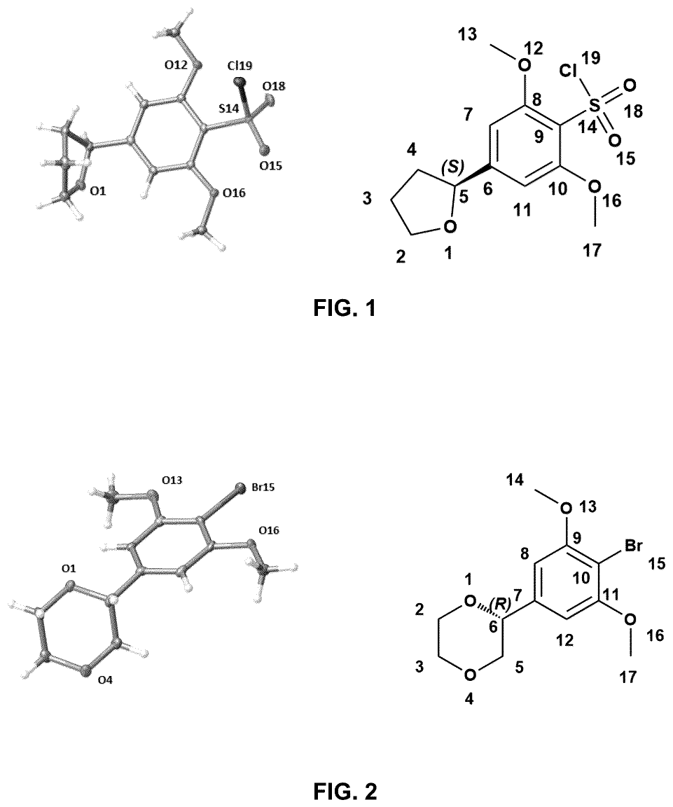

To a solution of 2-(3,5-dimethoxy-4-{[(4-methoxyphenyl)methyl]sulfanyl}phenyl)oxolane (140 g) (520 mg, 1.44 mmol) in acetic acid (10.8 mL) and water (3.61 mL) was added NCS (578 mg, 4.33 mmol) with the resulting reaction being stirred for 1 hr at rt. The crude reaction was diluted with water (15 mL) and extracted with EtOAc (3×15 mL). The combined organic extracts were washed with water (25 mL), dried over Na₂SO₄, and concentrated to afford an orange oil, which was purified by column chromatography (SiO₂, eluting with 5-75% EtOAc in heptane) to give compound 140h (231 mg, 52%) as a beige solid. The absolute stereochemistry of 140h was determined to be(S) by single-crystal X-ray crystallography. Crystals of Intermediate 140h were grown from DCM/Pentane, and data were collected in a nitrogen gas stream at 100 (2) K. See FIG. 1.

¹H NMR (400 MHZ, DMSO-d₆) δ 6.56 (s, 2H) 4.77 (t, J=7.09 Hz, 1H) 4.04-3.97 (m, 1H) 3.84-3.77 (m, 1H) 3.71 (s, 6H) 2.35-2.25 (m, 1H) 1.92 (quin, J=7.09 Hz, 2H) 1.72-1.60 (m, 1H).

### Step 4: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-[(2S)-oxolan-2-yl]benzene-1-sulfonamide (140j)

To a solution of N⁶-(5-cyclopropyl-1-(tetrahydro-2H-pyran-2-yl)-1H-pyrazol-3-yl)-5-methoxybenzo[d]isoxazole-3,6-diamine (8a) (180 mg, 0.46 mmol) and DMAP (5.67 mg, 0.046 mmol) in pyridine (1.16 mL) was added 2,6-dimethoxy-4-[(2S)-oxolan-2-yl]benzene-1-sulfonyl chloride (140h) (199 mg, 0.65 mmol). The solution turned a dark-orange color, and was stirred at rt for 18 hrs. The reaction was diluted with DCM (10 mL) and 1 M AcOH (10 mL) and extracted with DCM (3×8 mL). The combined organic extracts were washed with brine (20 mL), dried over Na₂SO₄, and concentrated to afford an orange oil that was purified by reverse phase HPLC (40-80% acetonitrile/water plus 10 mM ammonium acetate; 25 mL/min flow rate in 8 mins, 120 bar; Phenomenex Gemini 5 μm NX-C18 150×21.2 mm, 5 μm) to afford 140j (188 mg, 63%) as a white powder. ¹H NMR (400 MHZ, DMSO-d₆) δ 10.93 (s, 1H) 8.17 (s, 1H) 8.15 (s, 1H) 7.42 (s, 1H) 6.65 (s, 2H) 5.78 (s, 1H) 5.50 (dd, J=9.69, 2.44 Hz, 1H) 4.79 (t, J=7.13 Hz, 1H) 4.04-3.90 (m, 2H) 3.87 (s, 3H) 3.84-3.72 (m, 7H) 3.70-3.59 (m, 1H) 2.41-2.26 (m, 2H) 2.11-2.00 (m, 1H) 1.97-1.85 (m, 4H) 1.77-1.49 (m, 4H) 0.97 (dd, J=8.19, 2.44 Hz, 2H) 0.70-0.54 (m, 2H); LCMS m/z 639.9 (M+H)⁺.

### Step 5: Synthesis of N-{6-[(3-cyclopropyl-1H-pyrazol-5-yl)amino]-5-methoxy-1,2-benzoxazol-3-yl}-2,6-dimethoxy-4-[(2S)-oxolan-2-yl]benzene-1-sulfonamide (Example 140)

To a vial containing N-(6-{[5-cyclopropyl-1-(oxan-2-yl)-1H-pyrazol-3-yl]amino}-5-methoxy-1,2-benzoxazol-3-yl)-2,6-dimethoxy-4-[(2S)-oxolan-2-yl]benzene-1-sulfonamide (140j) (183 mg, 0.29 mmol) in DCM (1.43 mL) was added hydrogen chloride (2.86 mL, 4.0 M in dioxane, 11.4 mmol), and the reaction allowed to stir at rt for 48 hrs. The resulting solid was filtered and recrystallized from ACN to afford compound 140 (138 mg, 87%) as a white solid. ¹H NMR (400 MHz, DMSO-d₆) δ 10.93 (s, 1H) 8.11 (br. s, 1H) 8.03 (s, 1H) 7.42 (s, 1H) 6.65 (s, 2H) 5.82 (s, 1H) 4.78 (t, J=7.15 Hz, 1H) 4.03-3.95 (m, 1H) 3.88 (s, 3H) 3.84-3.78 (m, 1H) 3.76 (s, 6H) 2.37-2.27 (m, 1H) 1.96-1.83 (m, 3H) 1.65 (dq, J=12.15, 7.77 Hz, 1H) 0.99-0.89 (m, 2H) 0.73-0.65 (m, 2H)); LCMS m/z 556.9 (M+H)⁺; [a]_{D}²²=−14.8° (c 0.3, MeOH). The ee of N-{6-[(3-cyclopropyl-1H-pyrazol-5-yl)amino]-5-methoxy-1,2-benzoxazol-3-yl}-2,6-dimethoxy-4-[(2S)-oxolan-2-yl]benzene-1-sulfonamide (140) was confirmed to be >99% by chiral SFC (20% methanol plus 10 mM NH₃ in CO₂; 4 mL/min flow rate, 140 bar; Regis (S,S) Whelk-O1; 100×4.6 mm, 5 μm). The absolute stereochemical configuration of Example 140 was determined to be (S) owing to the x-ray structure of Intermediate 140h (Step 3) that demonstrated an absolute stereochemistry of (S) with none of the subsequent reactions of Intermediate 140h capable of impacting the stereochemical integrity of the chiral center. See FIG. 1.

### Example 141: Absolute stereochemical determination of N-{6-1 (3-cyclopropyl-1H-pyrazol-5-yl)amino]-5-methoxy-1,2-benzoxazol-3-yl}-2,6-dimethoxy-4-[(2R)-oxolan-2-yl]benzene-1-sulfonamide

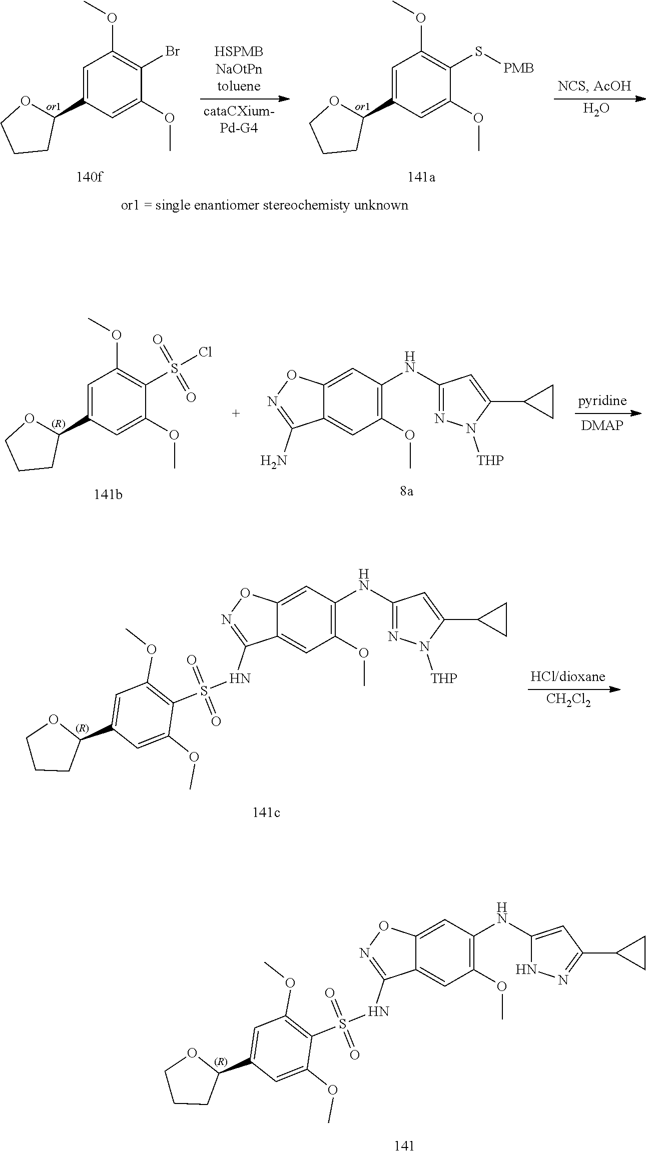

### Step 1: Synthesis of 2-(3,5-dimethoxy-4-{[(4-methoxyphenyl)methyl]sulfanyl}phenyl)oxolane (141a)

A vial was charged with 2-(4-bromo-3,5-dimethoxyphenyl)oxolane (140f) (565 mg, 1.97 mmol), CataCXium-Pd-G4 (146 mg, 0.19 mmol), 4-methoxybenzyl mercaptan (364 mg, 2.36 mmol), sodium 2-methylbutan-2-olate (1.63 g, 1.77 mL, 40% wt, 5.90 mmol) in toluene (19.7 mL). The vial was sealed, flushed three times with nitrogen. The reaction was heated at 100° C. for 18 hrs to give a light brown/orange solution. Sat. aq. NaHCO₃ (20 mL) was added to the stirred reaction mixture, which was extracted with EtOAc (3×20 mL). The combined organic extracts were washed with brine (100 mL), then dried over Na₂SO₄, filtered, and concentrated to give a yellow oil that was purified by column chromatography (SiO₂, eluting with 5-40% EtOAc in heptane) to give compound 141a (555 mg, 78%) as a thick orange gel. ¹H NMR (400 MHZ, DMSO-d₆) δ 7.12 (d, J=8.56 Hz, 2H) 6.79 (d, J=8.56 Hz, 2H) 6.58 (s, 2H) 4.77 (t, J=7.15 Hz, 1H) 4.05-3.97-(m, 1H) 3.90 (s, 2H) 3.85-3.79 (m, 1H) 3.77 (s, 6H) 3.70 (s, 3H) 2.30 (dq, J=12.47, 6.48 Hz, 1H) 1.93 (quin, J=7.09 Hz, 2H) 1.74-1.61 (m, 1H).

### Step 2: Synthesis of 2,6-dimethoxy-4-[(2R)-oxolan-2-yl]benzene-1-sulfonyl chloride (141b)

To a solution of 2-(3,5-dimethoxy-4-{[(4-methoxyphenyl)methyl]sulfanyl}phenyl)oxolane (141a) (550 mg, 1.53 mmol) in acetic acid (11.4 mL) and water (3.81 mL) was added NCS (611 mg, 4.58 mmol) with the resulting reaction being stirred for 1 hrs at rt. The crude reaction was diluted with water (15 mL) and extracted with EtOAc (3×15 mL). The combined organic extracts were washed with water (25 mL), brine (25 mL), dried over Na₂SO₄, and concentrated to afford an orange oil, which was purified by column chromatography (SiO₂, eluting with 5-75% EtOAc in heptane) to give compound 141b (256 mg, 55%) as a light yellow solid. The absolute stereochemistry of 141b was determined to be (R) owing to it being the opposite enantiomer of 140h that was studied by single-crystal X-ray crystallography. See FIG. 1. ¹H NMR (400 MHZ, DMSO-d₆) δ 6.56 (s, 2H) 4.77 (br. t, J=7.09 Hz, 1H) 3.97-4.05 (m, 1H) 3.76-3.85 (m, 1H) 3.72 (s, 6H) 2.25-2.35 (m, 1H) 1.92 (quin, J=7.09 Hz, 2H) 1.67 (dq, J=12.01, 7.77 Hz, 1H).

### Step 3: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-[(2R)-oxolan-2-yl]benzene-1-sulfonamide (141c)

To a solution of N⁶-(5-cyclopropyl-1-(tetrahydro-2H-pyran-2-yl)-1H-pyrazol-3-yl)-5-methoxybenzo[d]isoxazole-3,6-diamine (8a) (185 mg, 0.47 mmol) and DMAP (5.82 mg, 0.048 mmol) in pyridine (1.19 mL) was added 2,6-dimethoxy-4-[(2R)-oxolan-2-yl]benzene-1-sulfonyl chloride (141b) (249 mg, 0.81 mmol). The solution turned a dark-orange color, and was stirred at rt for 18 hrs. The reaction was diluted with DCM (10 mL) and 1 M AcOH (10 mL) and extracted with DCM (3×8 mL). The combined organic extracts were washed with brine (20 mL), dried over Na₂SO₄, and concentrated to afford an orange/brown oil that was dried under vacuum for 48 hrs to afford 141c (408 mg, >99% contains residual pyridine and acetic acid) as a beige solid that was used without further purification. ¹H NMR (400 MHZ, DMSO-d₆) δ 10.93 (s, 1H) 8.17 (s, 1H) 8.16 (s, 1H) 7.42 (s, 1H) 6.65 (s, 2H) 5.78 (s, 1H) 5.50 (dd, J=9.72, 2.38 Hz, 1H) 4.78 (t, J=7.15 Hz, 1H) 4.03-3.90 (m, 2H) 3.87 (s, 3H) 3.84-3.77 (m, 1H) 3.76 (s, 6H) 3.70-3.60 (m, 1H) 2.41-2.24 (m, 2H) 2.10-2.01 (m, 1H) 1.93-1.89 (m, 4H) 1.76-1.53 (m, 4H) 0.97 (dd, J=8.25, 2.38 Hz, 2H) 0.69-0.55 (m, 2H); LCMS m/z 641.0 (M+H)⁺.

### Step 4: Synthesis of N-{6-[(3-cyclopropyl-1H-pyrazol-5-yl)amino]-5-methoxy-1,2-benzoxazol-3-yl}-2,6-dimethoxy-4-[(2R)-oxolan-2-yl]benzene-1-sulfonamide (Example 141)

To a vial containing N-(6-{[5-cyclopropyl-1-(oxan-2-yl)-1H-pyrazol-3-yl]amino}-5-methoxy-1,2-benzoxazol-3-yl)-2,6-dimethoxy-4-[(2R)-oxolan-2-yl]benzene-1-sulfonamide (141c) (305 mg, 0.48 mmol) in DCM (2.38 mL) was added hydrogen chloride (4.77 mL, 4.0 M in dioxane, 19.1 mmol), and the reaction allowed to stir at rt for 18 hrs. The reaction was diluted with DCM (10 mL) and 1 M aqueous HCl (10 mL) and extracted with DCM (3×10 mL). The combined organic extracts were dried over Na₂SO₄, and concentrated to afford a clear oil that was purified by reverse phase HPLC (0-90% acetonitrile/water with 0.5% TFA; 25 mL/min flow rate in 8 mins, Phenomenex Gemini 5 μm NX-C18 150×21.2 mm, 5 μm) to afford 141 (216 mg, 82%) as a white powder. ¹H NMR (400 MHZ, DMSO-d₆) δ 10.91 (s, 1H) 8.08 (s, 1H) 8.04 (s, 1H) 7.40 (s, 1H) 6.65 (s, 2H) 5.79 (s, 1H) 4.79 (t, J=7.15 Hz, 1H) 4.03-3.94 (m, 1H) 3.87 (s, 3H) 3.84-3.78 (m, 2H) 3.76 (s, 6H) 2.36-2.26 (m, 1H) 1.97-1.81 (m, 3H) 1.65 (dq, J=12.15, 7.77 Hz, 1H) 0.97-0.88 (m, 2H) 0.71-0.62 (m, 2H); LCMS m/z 556.5 (M+H)⁺; [a]_{D}²²=+10.6° (c 0.3, MeOH). The ee of N-{6-[(3-cyclopropyl-1H-pyrazol-5-yl)amino]-5-methoxy-1,2-benzoxazol-3-yl}-2,6-dimethoxy-4-[(2R)-oxolan-2-yl]benzene-1-sulfonamide (141) was confirmed to be ˜99% by chiral SFC (20% methanol plus 10 mM NH₃ in CO₂; 4 mL/min flow rate, 140 bar; Regis (S,S) Whelk-O1; 100×4.6 mm, 5 μm). The absolute stereochemical configuration of Example 141 was determined to be (R) owing to Intermediate 141b being the opposite enantiomer of the x-ray structure of Intermediate 140h that demonstrated an absolute stereochemistry of(S) and with none of the subsequent reactions of Intermediate 141b capable of impacting the stereochemical integrity of the chiral center. See FIG. 1.

### Examples 142 and 143: N-{6-[(3-cyclopropyl-1H-pyrazol-5-yl)amino]-5-methoxy-1,2-benzoxazol-3-yl}-2,6-dimethoxy-4-(oxan-2-yl)benzene-1-sulfonamide

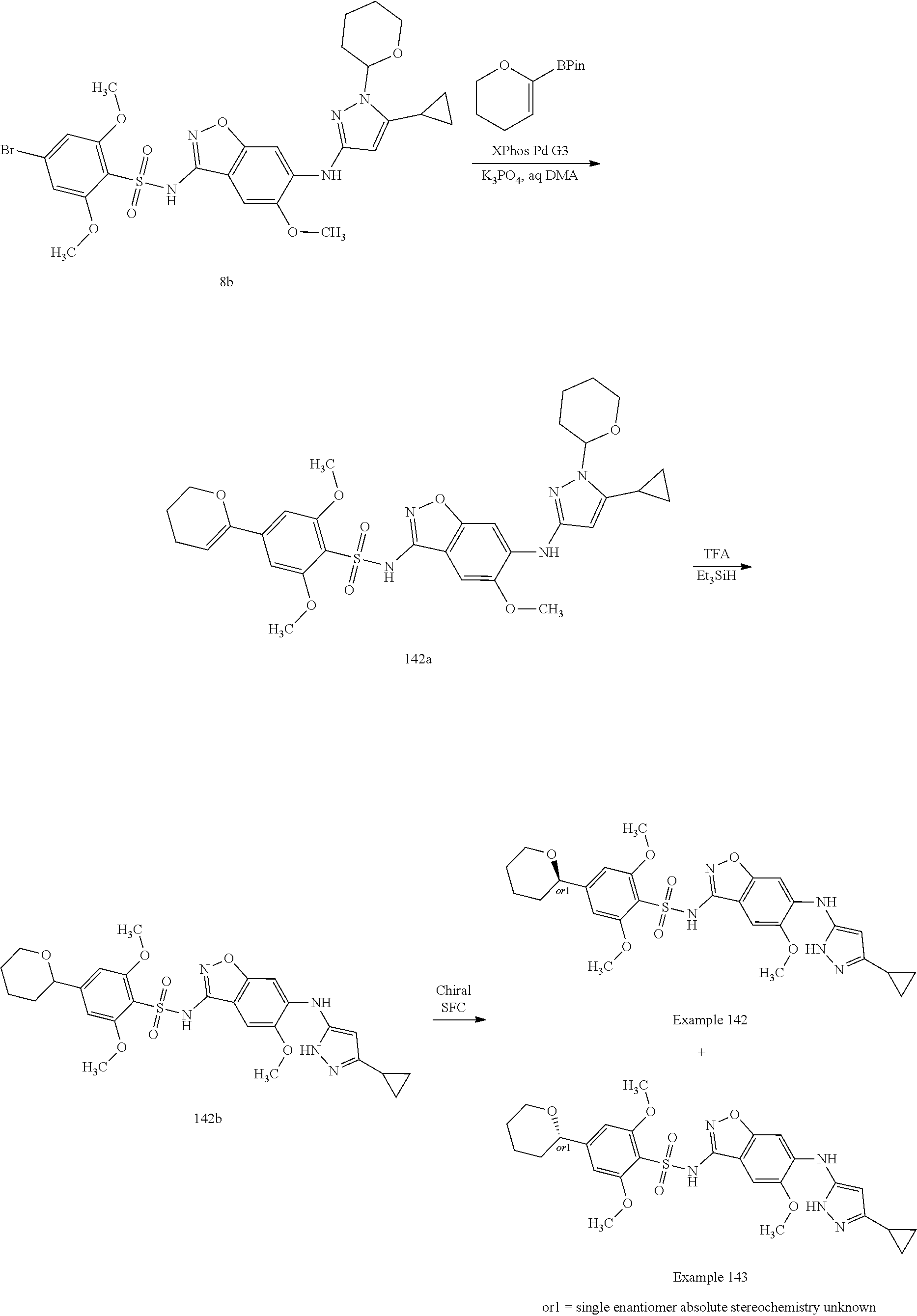

N-(6-{[5-cyclopropyl-1-(oxan-2-yl)-1H-pyrazol-3-yl]amino}-5-methoxy-1,2-benzoxazol-3-yl)-4-(3,4-dihydro-2H-pyran-6-yl)-2,6-dimethoxybenzene-1-sulfonamide (142a): To a solution of 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide (8b) (600 mg, 0.92 mmol) and 6-(4,4,5,5-tetramethyl-1,3,2-dioxaborolan-2-yl)-3,4-dihydro-2H-pyran (292 mg, 1.39 mmol) in DMA (5 mL) was added XPhos Pd G3 (78.3 mg, 0.09 mmol) and tripotassium phosphate (589 mg, 2.78 mmol) in H₂O (1.1 mL). The mixture was degassed, purged with nitrogen and heated at 80° C. for 3 hrs. The crude reaction mixture was combined with another batch (50 mg theoretical yield), filtered, concentrated under reduced pressure, and purified over silica gel (20 g, eluting with 50-100% EtOAc-Pet. ether, then 0-10% CH₃OH-DCM), which gave 142a as a yellow oil (673 mg, 100% yield). LCMS m/z 652.2 (M+H)⁺.

N-{6-[(3-cyclopropyl-1H-pyrazol-5-yl)amino]-5-methoxy-1,2-benzoxazol-3-yl}-2,6-dimethoxy-4-(oxan-2-yl)benzene-1-sulfonamide (Example 142 and 143): To a solution of N-(6-{[5-cyclopropyl-1-(oxan-2-yl)-1H-pyrazol-3-yl]amino}-5-methoxy-1,2-benzoxazol-3-yl)-4-(3,4-dihydro-2H-pyran-6-yl)-2,6-dimethoxybenzene-1-sulfonamide (142a) (730 mg, 1.12 mmol) in DCM (6 mL) and TFA (6 mL) was added triethylsilane (1.5 mL). After 30 mins, the brown solution turned black. An additional 6 mL of TFA was added and the crude reaction mixture was stirred for 3 hrs. The solvent was removed under reduced pressure and the resultant black oil was purified by reverse phase chromatography (C18 column, 150×40 mm, aq ammonium formate-acetonitrile mobile phase) which provided the product as a white solid (125 mg, 20% yield). LCMS m/z 570.3 (M+H)⁺. The enantiomers were separated by chiral SFC (30% methanol with 0.1% ammonium hydroxide in CO₂; 80 mL/min flow rate; Daicel Chiralcel OJ, 250 mm×30 mm, 10 μm).

N-{6-[(3-cyclopropyl-1H-pyrazol-5-yl)amino]-5-methoxy-1,2-benzoxazol-3-yl}-2,6-dimethoxy-4-(oxan-2-yl)benzene-1-sulfonamide (Example 142): The first eluting peak was isolated as a white solid (30 mg, 4.7% yield). ¹H NMR (400 MHZ, METHANOL-d₄) δ 7.61 (br s, 1H), 7.30 (s, 1H), 6.69 (s, 2H), 5.76 (s, 1H), 4.33 (dd, J=11.2, 1.9 Hz, 1H), 4.09-4.05 (m, 1H), 3.94 (s, 3H), 3.84 (s, 6H), 3.62-3.56 (m, 1H), 1.92-1.83 (m, 3H), 1.73-1.54 (m, 3H), 1.49-1.42 (m, 1H), 1.00-0.95 (m, 2H), 0.76-0.72 (m, 2H); LCMS m/z 570.3 (M+H)⁺; [a]_{D}²⁵=−17.43° (c, CH₃OH).

N-{6-[(3-cyclopropyl-1H-pyrazol-5-yl)amino]-5-methoxy-1,2-benzoxazol-3-yl}-2,6-dimethoxy-4-(oxan-2-yl)benzene-1-sulfonamide (Example 143): The second eluting peak (28 mg, white solid) was further purified by chiral SFC (35% methanol with 0.1% ammonium hydroxide in CO₂; 80 mL/min flow rate; Daicel Chiralcel OJ, 250 mm×30 mm, 10 μm) which provide the product as a white solid (15 mg, 2.3% yield). ¹H NMR (400 MHZ, METHANOL-d₄) δ 7.62 (br s, 1H), 7.30 (s, 1H), 6.69 (s, 2H), 5.76 (s, 1H), 4.33 (dd, J=11.2, 1.8 Hz, 1H), 4.09-4.06 (m, 1H), 3.94 (s, 3H), 3.83 (s, 6H), 3.62-3.56 (m, 1H), 1.95-1.86 (m, 3H), 1.72-1.56 (m, 3H), 1.49-1.42 (m, 1H), 1.00-0.95 (m, 2H), 0.76-0.72 (m, 2H); LCMS m/z 570.3 (M+H)⁺; [a]_{D}²⁵=+12.43° (c=0.19, CH₃OH).

### Example 144: N-{6-[(3-cyclopropyl-1H-pyrazol-5-yl)amino1-5-methoxy-1,2-benzoxazol-3-yl}-4-[(dimethylamino)methyl]-2,6-dimethoxybenzene-1-sulfonamide

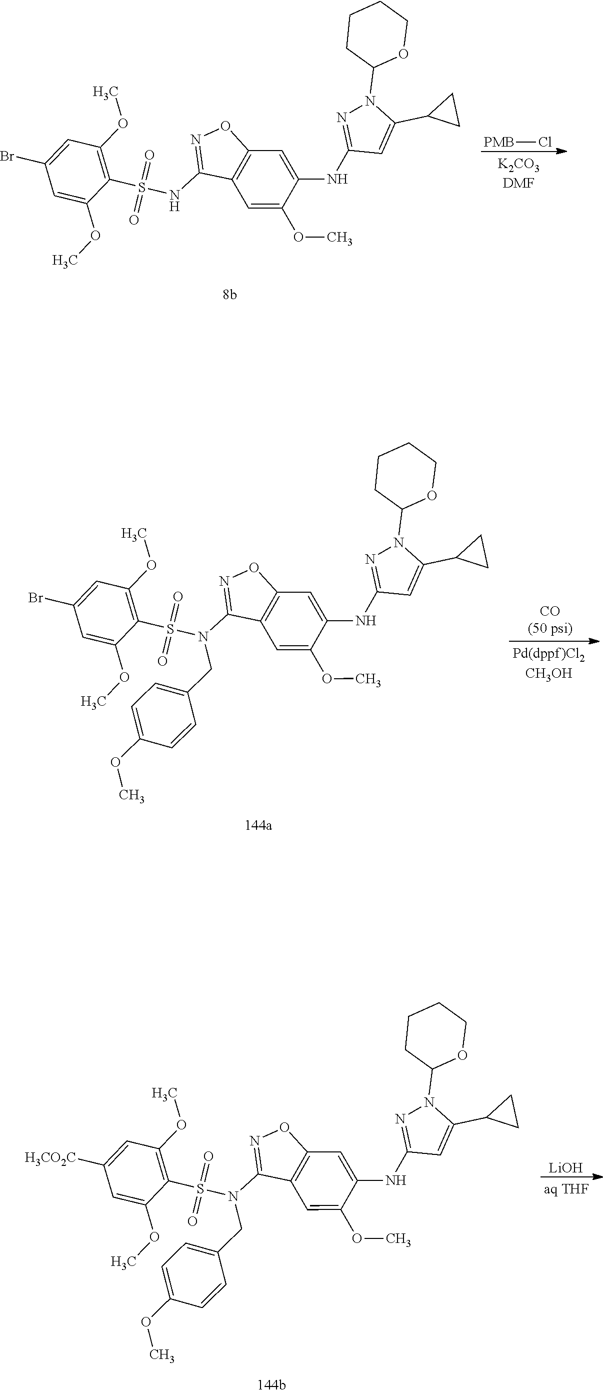

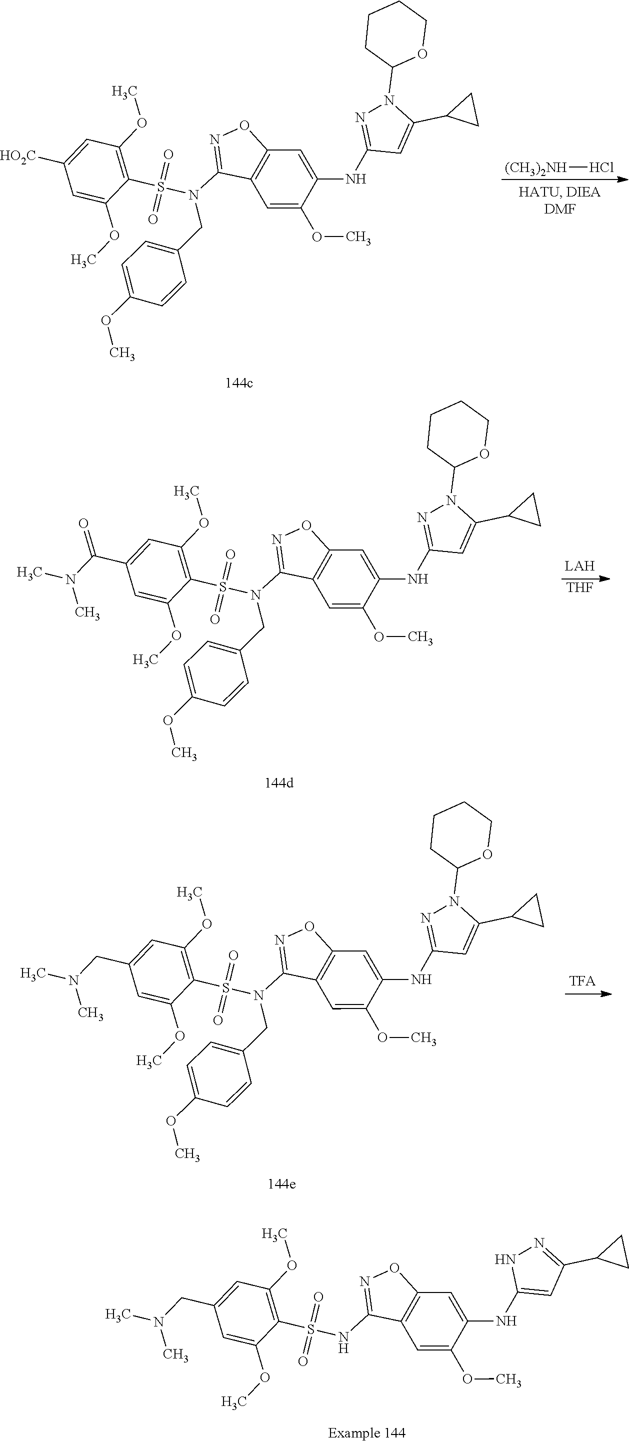

4-Bromo-N-(6-{[5-cyclopropyl-1-(oxan-2-yl)-1H-pyrazol-3-yl]amino}-5-methoxy-1,2-5 benzoxazol-3-yl)-2,6-dimethoxy-N-[(4-methoxyphenyl)methyl]benzene-1-sulfonamide (144a): To a mixture of 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide (8b) (930 mg, 1.43 mmol) and K₂CO₃ (793 mg, 5.74 mmol) in DMF (14.3 mL) was added 4-methoxybenzyl chloride (269 mg, 1.72 mmol) dropwise. The crude reaction mixture was stirred for 16 hrs at 80° C. The crude reaction mixture was added to ice water (10 mL) and a white solid formed. The mixture was extracted with EtOAc (5×3 mL) and the combined organic extracts were concentrated under reduced pressure to give an off-yellow solid. The crude product was purified over silica gel (40 g) and eluted with 0-50% EtOAc-Pet. ether which gave 144a as a white solid (930 mg, 84% yield). LCMS m/z 770 (M+H)⁺.

Methyl 4-{(6-{[5-cyclopropyl-1-(oxan-2-yl)-1H-pyrazol-3-yl]amino]-5-methoxy-1,2-benzoxazol-3-yl)[(4-methoxyphenyl)methyl]sulfamoyl}-3,5-dimethoxybenzoate (144b): To a mixture of 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide (144a) (930 mg, 1.21 mmol) in MeOH (60.5 mL) was added Pd(dppf)Cl₂ (177 mg, 0.242 mmol) and triethylamine (367 mg, 3.63 mmol). The mixture was degassed and purged with nitrogen two times then degassed and refilled with carbon monoxide twice. The reaction was stirred under an atmosphere of carbon monoxide (50 psi) at 65° C. for 16 hrs. The crude reaction mixture was concentrated and purified over silica gel (20 g) and eluted with 0-50% EtOAc-Pet. ether and gave 144b as a yellow solid (815 mg, 90% yield). LCMS m/z 748 (M+H)⁺.

4-{(6-{[5-Cyclopropyl-1-(oxan-2-yl)-1H-pyrazol-3-yl]amino}-5-methoxy-1,2-benzoxazol-3-yl)[(4-methoxyphenyl)methyl]sulfamoyl]-3,5-dimethoxybenzoic acid (144c): To a mixture of methyl 4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxybenzoate (144b) (815 mg, 1.09 mmol) in THF (10.9 mL) was added 2M LiOH—H₂O (1.36 mL, 2.72 mmol). The mixture was stirred at 25° C. for 3 hrs. The crude reaction mixture was diluted with H₂O (10 mL) at 0° C. then concentrated to remove THF. The resultant aqueous mixture was treated with 1M aqueous HCl until pH ˜3 then extracted with EtOAc (30 mL×3). The combined organic layers were washed with brine (30 mL) then dried (MgSO₄), filtered and concentrated to give 144c as a yellow solid (805 mg, 99% yield). ¹H NMR (400 MHZ, CHLOROFORM-d) δ 7.90 (s, 1H), 7.38 (d, J=8.6 Hz, 2H), 7.23 (s, 1H), 7.08 (s, 1H), 7.02 (s, 1H), 6.75 (d, J=8.6 Hz, 2H), 5.64 (s, 1H), 5.48 (dd, J=10.0, 2.4 Hz, 1H), 5.04 (s, 2H), 4.16-4.08 (m, 2H), 3.89 (s, 3H), 3.82-3.61 (m, 11H), 2.60-2.50 (m, 1H), 2.16-2.13 (m, 2H), 1.99-1.84 (m, 2H), 1.77-1.69 (m, 2H), 0.83-0.78 (m, 2H), 0.66-0.62 (m, 2H); LCMS m/z 734 (M+H)⁺.

4-{(6-{[5-Cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxy-N,N-dimethylbenzamide (144d): To a solution of 4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxybenzoic acid (144c) (400 mg, 0.545 mmol), DIEA (282 mg, 2.18 mmol) and dimethylamine hydrochloride (89 mg, 1.09 mmol) in DMF (2.73 mL) was added HATU (311 mg, 0.818 mmol). The mixture was stirred at rt for 16 hrs. The crude reaction mixture was diluted with ice water (20 mL), then extracted with EtOAc (20 mL×3). The combined organic layers were washed with brine (30 mL×2) then dried (MgSO₄), filtered and concentrated. The crude residue was purified over silica gel (20 g) and eluted with 0-100% EtOAc-Pet. ether, which gave 144d as a light orange solid (390 mg, 94% yield). LCMS m/z 761 (M+H)⁺.

N-(6-{[5-Cyclopropyl-1-(oxan-2-yl)-1H-pyrazol-3-yl]amino}-5-methoxy-1,2-benzoxazol-3-yl)-4-[(dimethylamino)methyl]-2,6-dimethoxy-N-[(4-methoxyphenyl)methyl]benzene-1-sulfonamide (144e): To a solution of 4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxy-N,N-dimethylbenzamide (144d) (50.0 mg, 0.066 mmol) in THF (0.66 mL) at 0° C. under nitrogen was added lithium aluminum hydride (2.74 mg, 0.0723 mmol). The crude reaction mixture was stirred for 50 mins at 0° C., then quenched with H₂O (3 mL) and extracted with EtOAc (5 mL×3). The combined organic extracts were washed with brine (10 mL) then dried (MgSO₄), filtered and concentrated to give 144e as a yellow gum (45 mg, 92% yield).

N-{6-[(3-cyclopropyl-1H-pyrazol-5-yl)amino]-5-methoxy-1,2-benzoxazol-3-yl}-4-[(dimethylamino)methyl]-2,6-dimethoxybenzene-1-sulfonamide (Example 144): A solution of N-(6-{[5-cyclopropyl-1-(oxan-2-yl)-1H-pyrazol-3-yl]amino}-5-methoxy-1,2-benzoxazol-3-yl)-4-[(dimethylamino)methyl]-2,6-dimethoxy-N-[(4-methoxyphenyl)methyl]benzene-1-sulfonamide (144e) (45 mg, 0.060 mmol) in TFA (1.0 mL) and DCM (1.0 mL) was stirred at rt for 16 hrs. The mixture was concentrated and purified by prep-HPLC (Boston Prime C18, 150×30 mm, 5 μm, H₂O with 0.1% ammonium hydroxide-ACN, gradient of 15-35% ACN over 10 mins, 35 mL/min) which gave Example 144 as a white solid (7.1 mg, 22% yield). ¹H NMR (METHANOL-d₄, 400 MHz) δ 7.54 (br s, 1H), 7.29 (s, 1H), 6.71 (s, 2H), 5.75 (s, 1H), 3.95 (s, 3H), 3.79 (s, 6H), 3.57 (s, 2H), 2.31 (s, 6H), 1.93-1.86 (m, 1H), 1.00-0.96 (m, 2H), 0.75-0.71 (m, 2H); LCMS m/z 543.3 (M+H)⁺.

### Examples 145 and 146: N-{6-[(3-cyclopropyl-1H-pyrazol-5-yl)amino]-5-methoxy-1,2-benzoxazol-3-yl}-4-[1,4-dioxan-2-yl]-2,6-dimethoxybenzene-1-sulfonamide

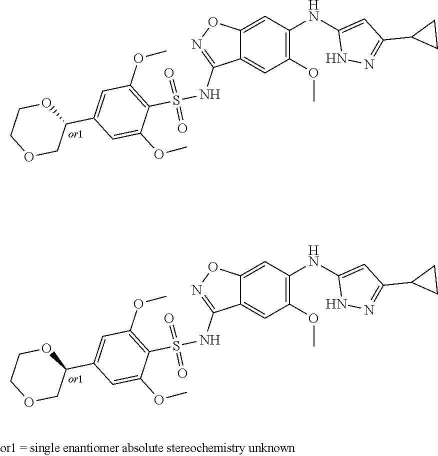 [SMILES: COc1cc2c(NS(=O)(=O)c3c(OC)cc([C@@H]4COCCO4)cc3OC)noc2cc1Nc1cc(C2CC2)n[nH]1 ; COc1cc2c(NS(=O)(=O)c3c(OC)cc([C@H]4COCCO4)cc3OC)noc2cc1Nc1cc(C2CC2)n[nH]1]

Examples 145 and 146 were made in a similar manner as Examples 142 and 143 using 5-(4,4,5,5-tetramethyl-1,3,2-dioxaborolan-2-yl)-2,3-dihydro-1,4-dioxine in place of 6-(4,4,5,5-tetramethyl-1,3,2-dioxaborolan-2-yl)-3,4-dihydro-2H-pyran. The enantiomers were separated by chiral SFC (60% isopropanol with 0.1% ammonium hydroxide in CO₂; 80 mL/min flow rate; Daicel Chiralcel OJ 250×30 mm, 10 μm).

### N-{6-[(3-cyclopropyl-1H-pyrazol-5-yl)amino]-5-methoxy-1,2-benzoxazol-3-yl}-4-[1,4-dioxan-2-yl]-2,6-dimethoxybenzene-1-sulfonamide (Example 145)

The first eluting peak was isolated as a white solid (46 mg, 19% yield). ¹H NMR (400 MHZ, CHLOROFORM-d) δ 7.78-7.71 (m, 1H), 7.47-7.42 (m, 1H), 7.06-6.98 (m, 1H), 6.62-6.54 (m, 2H), 5.76-5.64 (m, 1H), 4.61-4.52 (m, 1H), 4.00-3.96 (m, 3H), 3.96-3.93 (m, 1H), 3.92-3.90 (m, 6H), 3.89-3.78 (m, 3H), 3.75-3.65 (m, 1H), 3.40-3.28 (m, 1H), 1.90-1.82 (m, 1H), 1.04-0.95 (m, 2H), 0.78-0.70 (m, 2H); LCMS m/z 572.3 [M+H]⁺; [a]_{D}²⁶=−56° (c 0.1,MeOH). Example 145 was characterized by chiral SFC (40% isopropanol plus 0.05% DIPEA in CO₂; 4 mL/min flow rate, 103 bar, 35° C.; Chiralcel OJ-3; 50×4.6 mm, 3 μm) with a retention time of 0.961 min (peak 1).

N-{6-[(3-cyclopropyl-1H-pyrazol-5-yl)amino]-5-methoxy-1,2-benzoxazol-3-yl}-4-[1,4-dioxan-2-yl]-2,6-dimethoxybenzene-1-sulfonamide (Example 146): The second eluting peak was isolated as a white solid (39 mg, 16% yield). ¹H NMR (400 MHZ, CHLOROFORM-d) δ 7.82-7.72 (m, 1H), 7.50-7.38 (m, 1H), 7.07-6.96 (m, 1H), 6.64-6.54 (m, 2H), 5.77-5.62 (m, 1H), 4.65-4.47 (m, 1H), 4.00-3.96 (m, 3H), 3.96-3.93 (m, 1H), 3.92-3.90 (m, 6H), 3.89-3.77 (m, 3H), 3.76-3.65 (m, 1H), 3.40-3.27 (m, 1H), 1.89-1.82 (m, 1H), 1.04-0.95 (m, 2H), 0.79-0.68 (m, 2H); LCMS m/z 572.3 [M+H]⁺; [a]_{D}²⁶=+31° (c 0.1,MeOH). Example 146 was characterized chiral SFC (40% isopropanol plus 0.05% DIPEA in CO₂; 4 mL/min flow rate, 103 bar, 35° C.; Chiralcel OJ-3; 50×4.6 mm, 3 pm) with a retention time of 1.931 min (peak 2).

### Example 145 (Procedure to determine absolute stereochemistry)

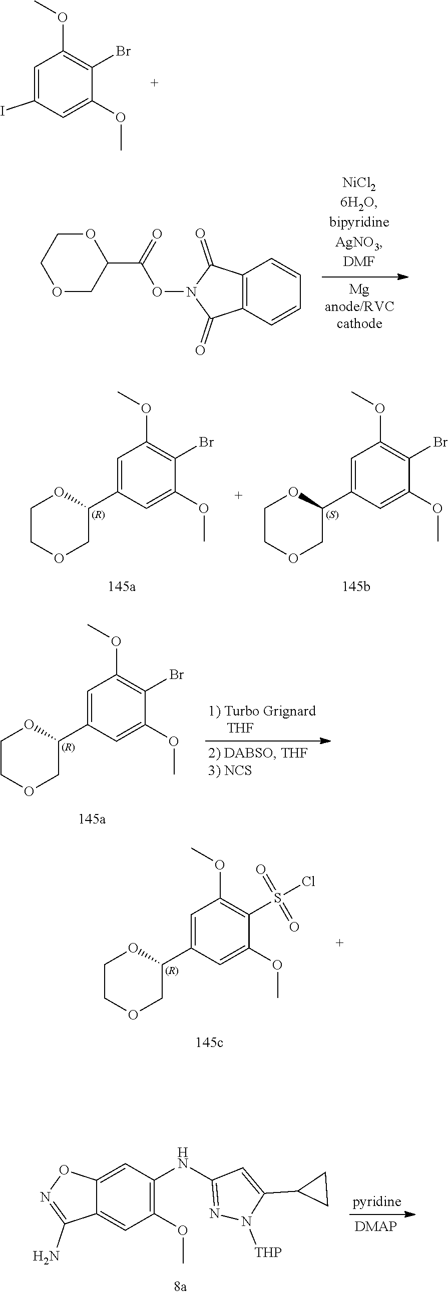

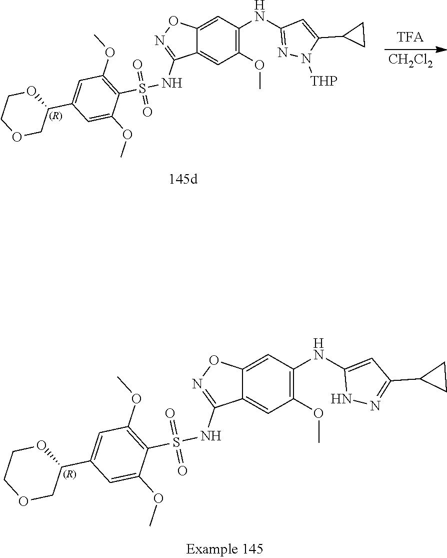

### Step 1: Synthesis of (2R)-2-(4-bromo-3,5-dimethoxyphenyl)-1,4-dioxane (145a) (small molecule x-ray for stereochemical determination) and (2S)-2-(4-bromo-3,5-dimethoxyphenyl)-1,4-dioxane (145b)

A vial was charged with 2-bromo-5-iodo-1,3-dimethoxybenzene (0.89 g, 2.6 mmol), 1,3-dioxoisoindolin-2-yl 1,4-dioxane-2-carboxylate (0.55 g, 2.0 mmol), nickel chloride hexahydrate (95 mg, 0.4 mmol), 2,2′-bipyridine (62 mg, 0.4 mmol), DMF (15.0 mL) and a stir bar. The mixture was stirred for about 5 mins, then silver nitrate (170 mg, 1.0 mmol) was added. The vial was closed with an IKA ElectraSyn 2.0 vial cap with a magnesium sacrificial anode (left side) and a reticulated vitreous carbon (RVC) cathode (right side), then the vial was immediately placed on an IKA ElectraSyn 2.0 stir plate. Electrolysis was set to 40 mA, 2.0 mmol, 4.0 F/mol. The mixture was allowed to stand for approximately 24 hrs. The crude reaction mixture was combined with an earlier batch of the same scale, diluted with water and extracted with MTBE (3×). The combined organic extracts were washed with brine then dried (Na₂SO₄), filtered and concentrated. The racemic mixture was purified by column chromatography (ISCO, SiO₂, eluting with 0-100% DCM in heptane) to give (477 mg, 39%) as a yellow solid. ¹H NMR (400 MHz, DMSO-d₆) δ 6.74 (s, 2H), 4.59 (dd, J=10.1, 2.7 Hz, 1H), 3.95-3.86 (m, 2H), 3.84 (s, 6H), 3.81-3.72 (m, 2H), 3.66-3.55 (m, 1H), 3.33-3.28 (m, 1H); LCMS m/z 303.6 (M+H)⁺. The enantiomers were separated by chiral SFC (10% methanol plus 10 mM NH₃ in CO₂; 4 mL/min flow rate, 160 bar, 25° C.; Lux Cell-1; 100×4.6 mm, 3 μm).

### 145a: (2R)-2-(4-bromo-3,5-dimethoxyphenyl)-1,4-dioxane

The first eluting peak was isolated as a white solid (188 mg, 31%, >95% ee). LCMS m/z 303.6 (M+H)⁺; [a]_{D}²²=−42.0° (c 0.1, MeOH). The absolute stereochemistry of 145a was determined to be (R) by single-crystal X-ray crystallography. Crystals of Intermediate 145a were grown from DCM/Pentane and data were collected in a nitrogen gas stream at 100 (2) K. See FIG. 2.

### 145b: (2S)-2-(4-bromo-3,5-dimethoxyphenyl)-1,4-dioxane

The second eluting peak was isolated as a white solid (193 mg, 32%, >95% ee). LCMS m/z 303.6 (M+H)⁺; [a]_{D}²²=+33.5° (c 0.1, MeOH). The absolute stereochemistry of 145b was determined to be(S) owing to it being the opposite enantiomer of 145a that was studied by single-crystal X-ray crystallography and determined to be (R). See FIG. 2.

### Step 2: Synthesis of 4-[(2R)-1,4-dioxan-2-yl]-2,6-dimethoxybenzene-1-sulfonyl chloride (145c)

To a vial was added (2R)-2-(4-bromo-3,5-dimethoxyphenyl)-1,4-dioxane (145a) (60 mg, 0.20 mmol) dissolved in THF (1.2 mL). Nitrogen was bubbled through the solution for 5 mins, then isopropylmagnesium chloride lithium chloride complex solution (69 mg, 0.37 mL, 1.3 M in THF, 2.4 eq, 0.48 mmol) was added and the reaction was stirred at 60° C. for 2.5 hrs. The reaction was cooled to rt and then added dropwise to a degassed solution of DABSO (0.12 g, 0.49 mmol) in THF (1.2 mL) at −40° C. The reaction was allowed to slowly warm to rt over 18 hrs. NCS (0.13 g, 0.95 mmol) was added, and the reaction was stirred at 40° C. for 20 mins then cooled to rt and diluted with EtOAc and water. The mixture was extracted with EtOAc (× 4) and washed with brine, then dried over sodium sulfate, filtered, and concentrated. The crude residue was purified by column chromatography (ISCO, SiO₂, eluting with 0-100% EtOAc in heptane) to give 145c (10 mg, 16%) as an oil. The crude oil was carried on.

### Step 3: Synthesis of N-(6-{[5-cyclopropyl-1-(oxan-2-yl)-1H-pyrazol-3-yl]amino}-5-methoxy-1,2-benzoxazol-3-yl)-4-[(2R)-1,4-dioxan-2-yl]-2,6-dimethoxybenzene-1-sulfonamide (145d)

To a vial was added 8a (10 mg, 27 μmol), 145c (10 mg, 32 μmol), DMAP (0.33 mg, 2.7 μmol), and pyridine (34 μL). The reaction was stirred at rt for 18 h. The crude reaction was diluted with 1 M AcOH and extracted with DCM (× 4). The combined organic layers were washed with brine then dried (Na₂SO₄), filtered and concentrated. The crude oil 145d was carried on. LCMS m/z 656.3 (M+H)⁺.

### Step 4: N-{6-[(3-cyclopropyl-1H-pyrazol-5-yl)amino]-5-methoxy-1,2-benzoxazol-3-yl}-4-[(2R)-1,4-dioxan-2-yl]-2,6-dimethoxybenzene-1-sulfonamide (145)

To a solution of 145d (10 mg, 15 μmol) in DCM (0.25 mL) was added dropwise TFA (0.21 g, 0.14 mL, 120 eq, 1.8 mmol). The reaction was sealed and stirred at rt for 18 hrs. The crude reaction was concentrated and analyzed by SFC to correlate retention time with structural assignment. Chiral SFC (40% isopropanol plus 0.05% DIPEA in CO₂; 4 mL/min flow rate, 103 bar, 35° C.; Chiralcel OJ-3; 50×4.6 mm, 3 μm) gave a retention time of 0.693 min (peak 1), which corresponds to 145. The absolute stereochemical configuration of Example 145 was determined to be (R) owing to the x-ray structure of Intermediate 145a (Step 1) that demonstrated an absolute stereochemistry of (R) with none of the subsequent reactions of Intermediate 145a capable of impacting the stereochemical integrity of the chiral center. See FIG. 2.

### Example 145 (stereospecific procedure): N-{6-[(3-cyclopropyl-1H-pyrazol-5-yl)amino]-5-methoxy-1,2-benzoxazol-3-yl}-4-[(2R)-1,4-dioxan-2-yl]-2,6-dimethoxybenzene-1-sulfonamide

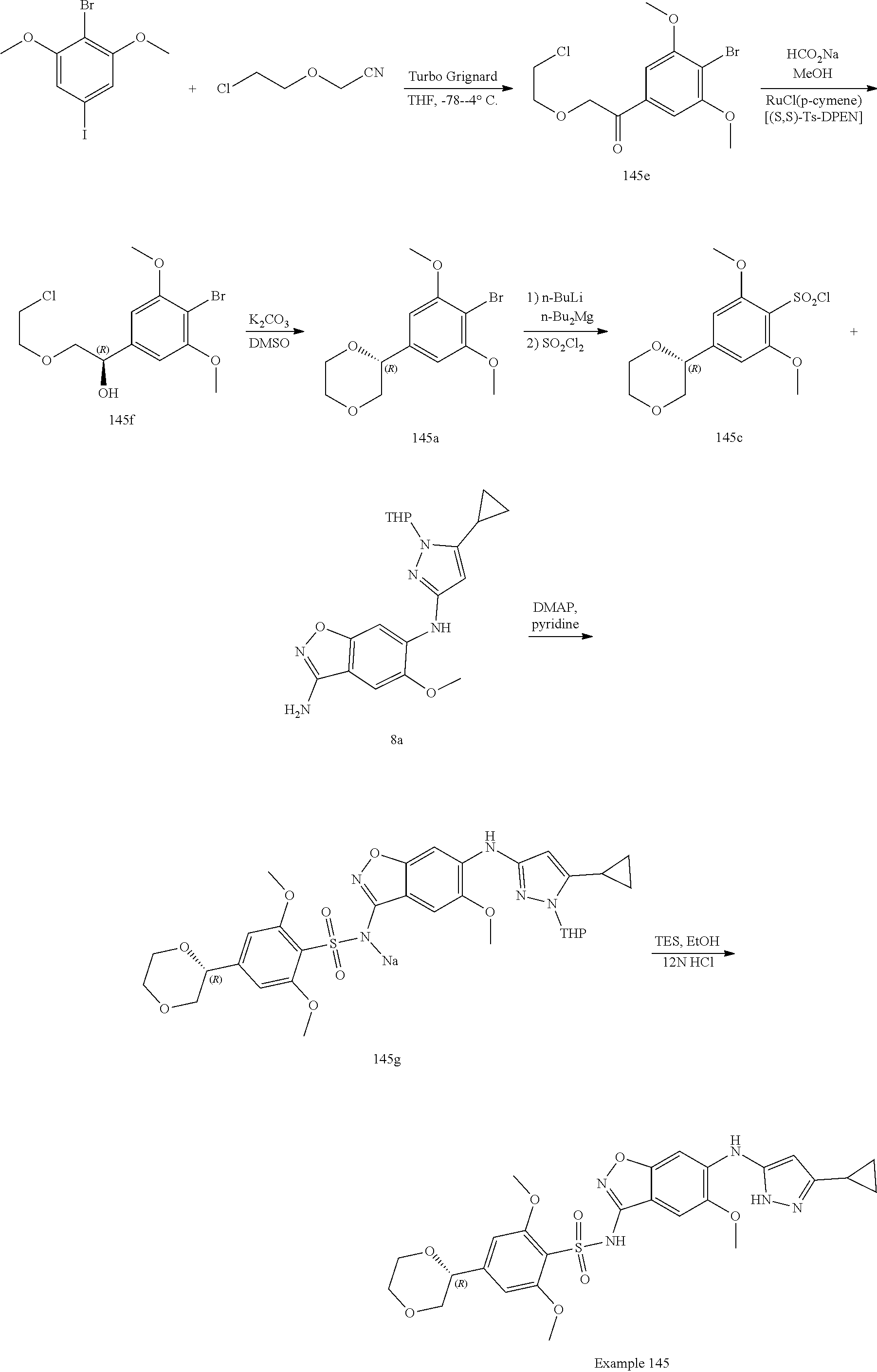

### Step 1: Synthesis of 1-(4-bromo-3,5-dimethoxyphenyl)-2-(2-chloroethoxy) ethan-1-one (145e)

A solution of 2-bromo-5-iodo-1,3-dimethoxybenzene (142.5 g, 415.51 mmol) in THF (1280 mL) was cooled in a dry ice/acetone bath and bubbled with N₂ for 15 mins. To the solution was added dropwise under nitrogen a solution of isopropylmagnesium chloride lithium chloride complex (1 M in THF, 457.06 mL). After stirring at −78° C. for 1 hr, 2-(2-chloroethoxy) acetonitrile (69.54 g, 581.71 mmol) in THF (143 mL) was added dropwise and stirred for 15 mins. The dry ice/acetone bath was replaced with a NaCl-ice bath and the reaction was stirred for 2.5 hrs. To the mixture was added water (500 mL) dropwise then the pH was adjusted to pH 3-4 with 1N aqueous HCl while keeping the reaction in the NaCl-ice bath. The aqueous phase was extracted with EtOAc (1 L×2). The organic layers were combined and washed with brine (1 L×2) then dried over MgSO₄, filtered, and concentrated. The crude residue was purified by column chromatography (SiO₂, 0-14% THF/Pet. ether). The resulting solid was slurried in MTBE (200 mL) at 25° C. for 3 hrs, then filtered and the filter cake was dried in vacuum to give 145e (150 g, 53.5%) as a white solid. ¹H NMR (400 MHZ, CDCl₃) δ 7.18 (s, 2H), 4.81 (s, 2H), 3.98 (s, 6H), 3.91-3.89 (m, 2H), 3.75-3.72 (m, 2H); LCMS m/z 339.0 (M+H)⁺.

### Step 2: Synthesis of (1R)-1-(4-bromo-3,5-dimethoxyphenyl)-2-(2-chloroethoxy) ethan-1-ol (145f)

To a solution of 145e (150 g, 444.32 mmol) in MeOH (2.5 L) was added sodium formate (60.43 g, 888.64 mmol, 48.04 mL) and chloro [(1R,2R)-(−)-2-amino-1,2-diphenylethyl](4-toluenesulfonyl)amido](p-cymene)ruthenium(II) (4.42 g, 6.93 mmol). The mixture was cooled in an ice bath and degassed via three vacuum-purge sequences. The resultant yellow suspension was stirred at 25° C. for 16 hrs. The mixture was concentrated to about 1.5 L MeOH to give a yellow solid precipitate, which was filtered. To the filtrate was added 1.5 L of H₂O with stirring, and additional 500 mL of H₂O were added to give a brown solid suspension. The brown solid was filtered and rinsed with H₂O. The yellow solid and the brown solid were combined then 500 mL of H₂O were added. The slurry was stirred at 25° C. for 1 hr, then filtered and rinsed with H₂O (250 mL×3) to provide 145f (150 g, 99.17%) as a gray solid. ¹H NMR (400 MHZ, CDCl₃) δ 6.63 (s, 2H), 4.89 (dd, J=3.1, 8.8 Hz, 1H), 3.91 (s, 6H), 3.82 (dt, J=3.6, 5.5 Hz, 2H), 3.75-3.64 (m, 3H), 3.58-3.44 (m, 1H), 2.98 (br s, 1H).

### Step 3: Synthesis of (2R)-2-(4-bromo-3,5-dimethoxyphenyl)-1,4-dioxane (145a)

To a solution of 145f (150 g, 441.68 mmol) in DMSO (1 L) was added K₂CO₃ (183.13 g, 1.33 mol) in H₂O (500 mL). The mixture was heated to 80° C. for 16 hrs then cooled to 25° C. H₂O (2 L) was added and the mixture was stirred at 25° C. for 1 hr then filtered and rinsed with H₂O (250 mL×5) to give a gray solid. The solid was dissolved in EtOAc (800 mL) then dried with MgSO₄, filtered and concentrated to give 145a (100 g, 74%) as an off-white solid. ¹H NMR (400 MHz, CDCl₃) δ 6.56-6.43 (m, 2H), 4.58-4.43 (m, 1H), 3.92-3.86 (m, 2H), 3.85-3.82 (m, 6H), 3.81-3.62 (m, 3H), 3.43-3.27 (m, 1H). LCMS m/z 305.1 (M+H)⁺.

### Step 4: Synthesis of 4-[(2R)-1,4-dioxan-2-yl]-2,6-dimethoxybenzene-1-sulfonyl chloride (145c)

Dibutylmagnesium (1 M in heptane, 362.86 mL) was added to n-butyllithium (2.5 M in hexanes, 72.57 mL) and stirred at 25° C. for 30 mins. The mixture was cooled to −60° C., then a solution of 145a (55 g, 181.43 mmol, 1 eq) in THF (250 mL) was added and stirring was continued at −60° C. for 3 hrs. The resulting mixture was added to a −60° C. solution of sulfuryl chloride (97.95 g, 725.72 mmol, 72.56 mL) in toluene (550 mL) and stirred for 30 mins. The reaction was quenched with H₂O (1 L) and extracted with EtOAc (250 ml×3) then washed with brine (500 ml×3) and dried (MgSO₄), filtered and concentrated. The crude residue was purified by column chromatography (SiO₂, 0-25% THF/Pet. ether) to give compound 145c (43.2 g, 70%) as an off-white solid. ¹H NMR (400 MHZ, CDCl₃) δ 6.63 (s, 2H), 4.63 (dd, J=2.6, 10.1 Hz, 1H), 3.99 (s, 6H), 3.98-3.79 (m, 4H), 3.78-3.68 (m, 1H), 3.39 (dd, J=10.2, 11.6 Hz, 1H).

### Step 5: sodium (6-{[5-cyclopropyl-1-(oxan-2-yl)-1H-pyrazol-3-yl]amino}-5-methoxy-1,2-benzoxazol-3-yl){4-[(2R)-1,4-dioxan-2-yl]-2,6-dimethoxybenzene-1-sulfonyl}azanide (145 g)

To a solution of compound 8a (25 g, 67.67 mmol) and DMAP (826.78 mg, 6.77 mmol) in pyridine (150 mL) was added 4 Å Molecular Sieves (75 g) The reaction was purged with nitrogen then 145c (28.4 g, 87.98 mmol) was added and stirring was continued at rt for 16 hrs. The reaction mixture was filtered and rinsed with MeOH. The resulting filtrate was concentrated then diluted in MeOH (450 mL) and NaOH (8.12 g, 203.02 mmol) was added. Upon addition of NaOH a yellow solid crashed out of solution. The mixture was heated at 60° C. for 1 hr, cooled to 25° C., then 0° C. The resulting mixture was filtered and rinsed with cold MeOH (100 mL×3) then dried to give 145 g (43 g, 94% yield) as a white solid. ¹H NMR (400 MHZ, DMSO-d₆) δ 8.05-7.97 (m, 1H), 7.80-7.71 (m, 1H), 7.09-7.02 (m, 1H), 6.67-6.56 (m, 2H), 5.78-5.70 (m, 1H), 5.53-5.44 (m, 1H), 4.57-4.46 (m, 1H), 3.95-3.82 (m, 6H), 3.78-3.68 (m, 2H), 3.61 (s, 7H), 3.31-3.24 (m, 1H), 2.41-2.27 (m, 1H), 2.09-2.02 (m, 1H), 1.96-1.87 (m, 2H), 1.78-1.65 (m, 1H), 1.61-1.51 (m, 2H), 1.03-0.90 (m, 2H), 0.55-0.53 (m, 1H), 0.70-0.52 (m, 2H); LCMS m/z 656.4 (M+H−Na)⁺.

### Step 6: N-{6-[(3-cyclopropyl-1H-pyrazol-5-yl)amino]-5-methoxy-1,2-benzoxazol-3-yl}-4-[(2R)-1,4-dioxan-2-yl]-2,6-dimethoxybenzene-1-sulfonamide (Example 145)

To a cooled (0˜5° C.) solution of 145 g (43 g, 63.45 mmol) in EtOH (450 mL) was added triethylsilane (14.76 g, 126.90 mmol, 20.27 mL) and HCl (12 M, 317.25 mL). The mixture was stirred at 25° C. for 16 hrs then concentrated and poured into a sat. aq. solution of NaHCO₃ (1.5 L) to adjust to pH ˜7. The aqueous layer was further extracted with DCM (550 mL×3) and the combined organic layers were dried over MgSO₄, filtered and concentrated. The resulting crude solid was dissolved in MeOH (150 mL) and heated to 70° C. for 5 hrs. The mixture was filtered, and the filter cake was washed with MeOH (100 mL×3) then dried to give 145 (30.3 g, 83%) as off-white solid. ¹H NMR (400 MHZ, DMSO-d₆) δ 12.01-11.86 (m, 1H), 11.05-10.89 (m, 1H), 8.16-8.11 (m, 1H), 8.07-8.03 (m, 1H), 7.43-7.34 (m, 1H), 6.81-6.69 (m, 2H), 5.82-5.75 (m, 1H), 4.62-4.53 (m, 1H), 3.93-3.87 (m, 5H), 3.79-3.69 (m, 8H), 3.61-3.51 (m, 1H), 3.31-3.22 (m, 1H), 1.95-1.77 (m, 1H), 0.97-0.84 (m, 2H), 0.73-0.61 (m, 2H); LCMS m/z 572.2 (M+H)⁺.

Structural assignment of 145 was confirmed by chiral SFC (40% isopropanol plus 0.05% DIPEA in CO₂; 4 mL/min flow rate, 103 bar, 35° C.; Chiralcel OJ-3; 50×4.6 mm, 3 μm) with a retention time of 0.686 min (peak 1). [a]_{D}²²=−21.3° (c 0.31, CH₃OH).

### Example 146 (stereospecific procedure): N-{6-[(3-cyclopropyl-1H-pyrazol-5-yl)amino]-5-methoxy-1,2-benzoxazol-3-yl}-4-[(2S)-1,4-dioxan-2-yl]-2,6-dimethoxybenzene-1-sulfonamide

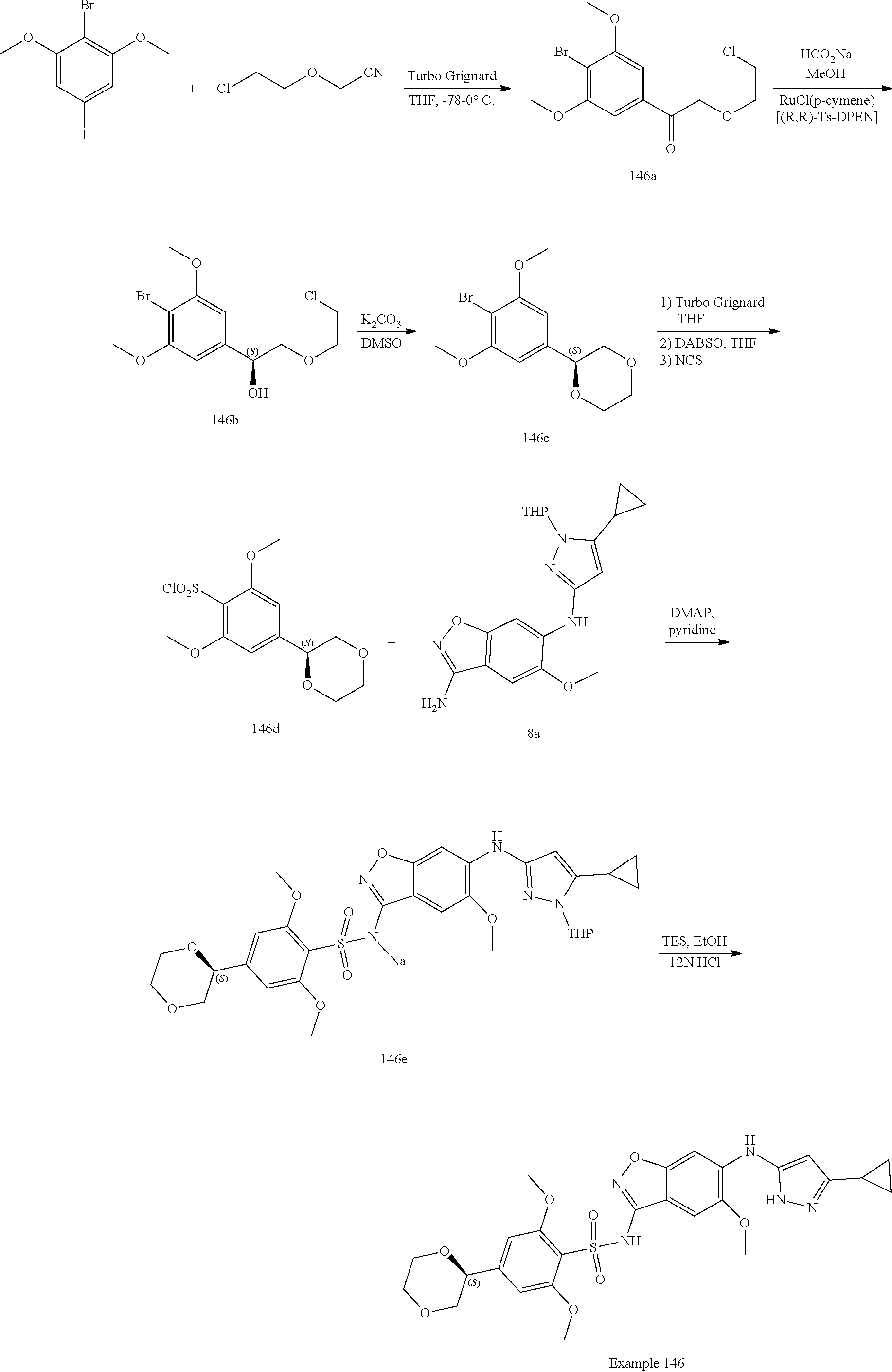

### Step 1: Synthesis of 1-(4-bromo-3,5-dimethoxyphenyl)-2-(2-chloroethoxy) ethan-1-one (146a)

A solution of 2-bromo-5-iodo-1,3-dimethoxybenzene (40.0 g, 117 mmol) in THF (360 mL) at −78° C. was sparged with nitrogen for 15 mins. To the solution was added dropwise under nitrogen a solution of isopropylmagnesium chloride lithium chloride complex (18.6 g, 128 mmol; 98.7 mL of a 1.30 M solution in THF). After stirring at −78° C. for 1 hr, 2-(2-chloroethoxy) acetonitrile (20.9 g, 175 mmol) was added dropwise as a solution in THF (40 mL). The cooling bath was replaced with a NaCl-ice bath (−4° C.) where it was allowed to warm to 0° C. over 3 hrs. The mixture was partitioned between an equal volume of water and EtOAc, and the pH of the mixture was adjusted to 6.0 by the stirred, slow addition of 1 N aqueous HCl (˜225 mL) while maintaining ice bath temperature. The aqueous layer was extracted with two additional volumes of EtOAc, and the combined organic extracts were washed with water (2×) followed by brine. Concentration under vacuum afforded a yellow solid, which was then stirred as a slurry in MeOH (100 mL) at rt for 1 h. The solid was collected via filtration, rinsing with cold MeOH (25 mL) to give batch 1. The resulting filtrate was concentrated and purified by column chromatography (120 g, silica gel, ISCO, 0-100% EtOAc/heptanes) to give batch 2. The two batches were combined to afford 146a (25.0 g, 63%) as a white solid. ¹H NMR (400 MHZ, CDCl₃) 0 7.19 (s, 2H), 4.80 (s, 2H), 3.98 (s, 6H), 3.91-3.87 (m, 2H), 3.75-3.71 (m, 2H).

### Step 2: Synthesis of (1S)-1-(4-bromo-3,5-dimethoxyphenyl)-2-(2-chloroethoxy) ethan-1-ol (146b)

A mixture of 1-(4-bromo-3,5-dimethoxyphenyl)-2-(2-chloroethoxy) ethan-1-one 146a (14.0 g, 41.5 mmol), sodium formate (5.64 g, 89.2 mmol), and chloro [(1R,2R)-(−)-2-amino-1,2-diphenylethyl](4-toluenesulfonyl)amido](p-cymene)ruthenium(II) (412 mg, 647 μmol) was cooled to −4° C. in a sodium chloride-ice bath, and MeOH (210 mL) was added. The suspension was degassed via three vacuum-purge sequences and the resultant yellow suspension was stirred overnight at ice bath temperature to afford an orange solution with a white precipitate. The solid was collected via filtration and the filtrate was concentrated under vacuum to approximately 100 mL. The resultant yellow solid was collected via filtration. To the filtrate was added with stirring 200 ml of water, followed by another 200 ml of water. The resultant brown precipitate was collected via filtration and rinsed with water. The prior white and yellow precipitates were combined and stirred as a slurry in 100 ml of water, collected via filtration, rinsed with water, and dried to afford 146b (9.44 g, 67%) as a pale-yellow solid. The filtrate was added to the brown solid from above, and the mixture was partitioned with an equal volume of EtOAc. The aqueous portion was extracted twice with EtOAc, and the combined extracts were washed with brine, dried over Na₂SO₄, and concentrated to afford additional 146b (4.54 g, 32%) as a brown solid. Combined yield: 14.0 g (99%). ¹H NMR (400 MHZ, CDCl₃) δ 6.64 (s, 2H), 4.90 (td, J=2.8, 8.6 Hz, 1H), 3.92 (s, 6H), 3.88-3.78 (m, 2H), 3.74-3.66 (m, 3H), 3.52 (dd, J=8.8, 9.8 Hz, 1H), 2.86 (d, J=2.4 Hz, 1H). Chiral assessment of each sample lot was performed individually via Chiral Pak IK-3 4.6×100 mm×3 mm column, eluting with 10% MeOH in CO₂ 120 bar 4 mL/mins, with enantiomer 1 eluting at 1.91 mins and enantiomer 2 at 2.33 mins. The yellow solid from above was obtained with 98% ee; the brown solid with 83% ee.

### Step 3: Synthesis of (2S)-2-(4-bromo-3,5-dimethoxyphenyl)-1,4-dioxane (146c)

To(S)-1-(4-bromo-3,5-dimethoxyphenyl)-2-(2-chloroethoxy) ethan-1-ol 146b (9.44 g, 27.8 mmol) in DMSO (69.9 mL) was added K₂CO₃ (11.5 g, 83.4 mmol) as a solution in water (35 mL). The mixture was heated at 75° C. overnight, then cooled to rt and stirred for 1 hr. Water (140 mL) was added slowly, and the resultant mixture was stirred for 1 hr. The solid was collected via filtration, rinsed with water, and dried to afford 146c (8.0 g, 95%) as a white solid. ¹H NMR (400 MHz, CDCl₃) δ 6.58 (s, 2H), 4.60 (dd, J=2.8, 10.1 Hz, 1H), 4.04-3.67 (m, 11H), 3.44 (dd, J=10.2, 11.7 Hz, 1H); LCMS m/z 305.0 (M+H)⁺.

### Step 4: Synthesis of 4-[(2S)-1,4-dioxan-2-yl]-2,6-dimethoxybenzene-1-sulfonyl chloride (146d)

All glassware and stir bars were oven-dried >100° C. overnight under vacuum. SM and DABSO were dried under vacuum overnight.

To (2S)-2-(4-bromo-3,5-dimethoxyphenyl)-1,4-dioxane 146c (6.94 g, 22.9 mmol) in a 200 mL round bottom flask under nitrogen was added degassed THF (43 mL). Isopropylmagnesium chloride lithium chloride complex solution (7.98 g, 55.0 mmol, 42.3 mL of a 1.3 M in THF) was added slowly. The mixture was heated to 60° C. for 2 hrs. The resultant yellow solution was cooled to rt and added via cannula to a stirred suspension of DABSO (14.3 g, 59.6 mmol) in degassed THF (46 mL) at −40° C. in a two-neck round bottom flask. The mixture was allowed to warm to 0° C. over 1 hr, at which point the cooling bath was removed and the mixture brought to rt. N-Chlorosuccinimide (9.12 g, 68.7 mmol) was added, and the resultant white slurry was stirred for 1 hr. The brown mixture was then partitioned between EtOAc (200 mL) and water (100 mL). The aqueous portion was extracted with EtOAc (2×45 mL), and the combined extracts were washed with water, dried over Na₂SO₄ and concentrated to afford a pale-yellow solid. The solid was slurried with MTBE, collected via filtration, and rinsed with additional MTBE to afford 146d (3.96 g, 54%) as a white solid. ¹H NMR (400 MHZ, CDCl₃) δ 6.64 (s, 2H), 4.63 (dd, J=2.8, 10.1 Hz, 1H), 4.04-3.68 (m, 11H), 3.39 (dd, J=10.0, 11.6 Hz, 1H).

### Step 5: Synthesis of sodium (6-{[5-cyclopropyl-1-(oxan-2-yl)-1H-pyrazol-3-yl]amino}-5-methoxy-1,2-benzoxazol-3-yl) {4-[(2S)-1,4-dioxan-2-yl]-2,6-dimethoxybenzene-1-sulfonyl}azanide (146e)

A mixture of N⁶-[5-cyclopropyl-1-(oxan-2-yl)-1H-pyrazol-3-yl]-5-methoxy-1,2-benzoxazole-3,6-diamine 8a (8.77 g, 23.7 mmol) and(S)-4-(1,4-dioxan-2-yl)-2,6-dimethoxybenzenesulfonyl chloride 146d (9.58 g, 29.7 mmol) was evacuated and backfilled with nitrogen, then anhydrous pyridine (24 mL) was added and the mixture was heated at 60° C. for 2 hrs. The resultant solution was concentrated and, with stirring, was diluted sequentially with MeOH (70 mL) and sodium hydroxide (17.8 mL of a 4 M solution; 71.2 mmol). The dark mixture was heated to 60° C. for 1 hr during which time a yellow suspension formed. The mixture was cooled to rt, and the precipitate was collected via filtration and rinsed with MeOH to afford 146e (8.68 g, 52%) as an off white solid. ¹H NMR (400 MHZ, D₂O) δ 7.59 (s, 1H), 7.05 (s, 1H), 6.72 (s, 2H), 5.79 (s, 1H), 5.67 (dd, J=2.3, 10.8 Hz, 1H), 4.04 (br d, J=2.6 Hz, 1H), 3.99-3.81 (m, 8H), 3.79-3.72 (m, 2H), 3.66 (s, 6H), 3.56 (dd, J=10.6, 11.8 Hz, 1H), 2.37-2.29 (m, 1H), 2.09-1.99 (m, 1H), 1.98-1.83 (m, 2H), 1.78-1.56 (m, 3H), 1.00 (dd, J=2.4, 8.7 Hz, 2H), 0.78-0.63 (m, 2H), LCMS m/z 656.3 (M+H−Na)⁺.

### Step 6: Synthesis of N-{6-[(3-cyclopropyl-1H-pyrazol-5-yl)amino]-5-methoxy-1,2-benzoxazol-3-yl}-4-[(2S)-1,4-dioxan-2-yl]-2,6-dimethoxybenzene-1-sulfonamide (Example 146)

A stirred suspension of sodium (6-{[5-cyclopropyl-1-(oxan-2-yl)-1H-pyrazol-3-yl]amino}-5-methoxy-1,2-benzoxazol-3-yl){4-[(2S)-1,4-dioxan-2-yl]-2,6-dimethoxybenzene-1-sulfonyl}azanide 146e (31.2 g, 46.0 mmol) and triethylsilane (10.7 g, 92.0 mmol) in ethanol (460 mL) was cooled in an ice bath. To the mixture was added dropwise by addition funnel 12 N HCl (230 mL, 2.76 mol). The mixture was warmed to rt and stirred for 4 hrs. The volume was reduced to 150 mL, and the material was then diluted with DCM (500 mL) and water (200 mL). The pH was neutralized by the careful addition of sat. aq NaHCO₃, affording a milky suspension. The resultant mixture was partitioned by the addition of DCM (450 mL), water (200 mL), brine (100 mL) and MeOH (250 mL). The layers were split, and the aqueous portion was extracted sequentially with DCM to afford additional product. Combined organic extracts were dried over Na₂SO₄ to afford Example 146 (24.9 g, 95%) as a pale-yellow solid with 93% NMR potency. ¹H NMR (400 MHZ, DMSO-d₆) δ11.89 (br s, 1H), 10.94 (br s, 1H), 8.10 (br s, 1H), 8.02 (s, 1H), 7.37 (s, 1H), 6.71 (s, 2H), 5.77 (s, 1H), 4.56 (dd, J=2.5, 10.0 Hz, 1H), 3.92-3.83 (m, 5H), 3.78-3.68 (m, 8H), 3.62-3.50 (m, 1H), 3.26 (dd, J=10.2, 11.4 Hz, 1H), 1.85 (tt, J=5.0, 8.4 Hz, 1H), 0.95-0.87 (m, 2H), 0.70-0.62 (m, 2H); LCMS m/z 572.1 (M+H)⁺.

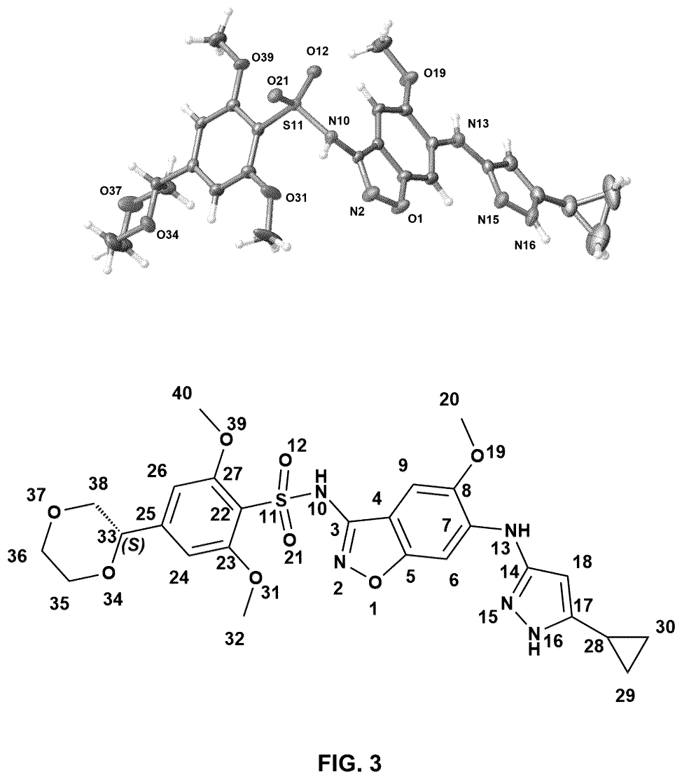

Structural assignment of 146 was confirmed by chiral SFC (40% isopropanol plus 0.05% DIPEA in CO₂; 4 mL/min flow rate, 103 bar, 35° C.; Chiralcel OJ-3; 50×4.6 mm, 3 □m) with a retention time of 1.327 min (peak 2), and optical rotation [a]_{D}²²=+20.6° (c 0.28, CH₃OH). The absolute stereochemistry of Example 146 was determined to be(S) by single crystal X-ray diffraction studies. Crystals of Example 146 were grown in MeOH/H₂O and data were collected in a nitrogen gas stream at 100 (2) K. See FIG. 3.

### Examples 147 and 148: N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide

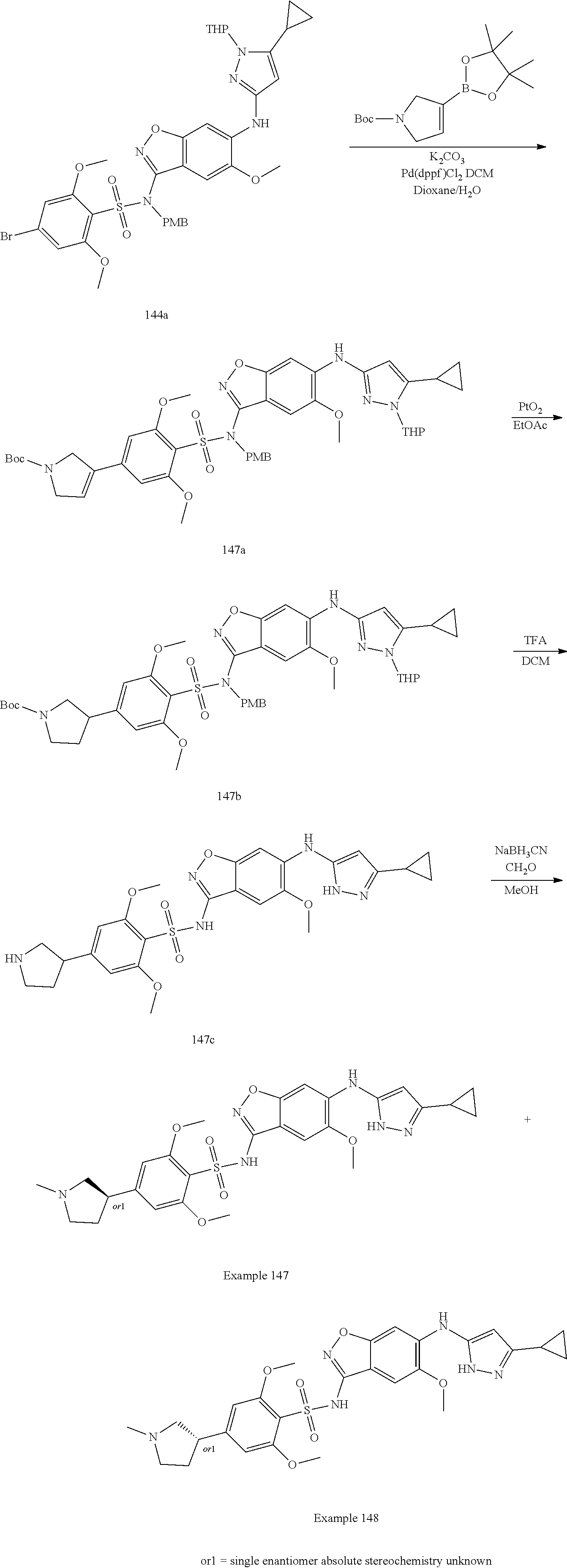

### Step 1: Synthesis of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)-2,5-dihydro-1H-pyrrole-1-carboxylate (147a)

To a mixture of 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide (144a) (800 mg, 1.04 mmol) in dioxane (8.0 mL) and H₂O (2.0 mL) was added tert-butyl 3-(4,4,5,5-tetramethyl-1,3,2-dioxaborolan-2-yl)-2,5-dihydro-1H-pyrrole-1-carboxylate (323 mg, 1.09 mmol), K₂CO₃ (432 mg, 3.12 mmol) and Pd(dppf)Cl₂ complexed with DCM (1:1) (85.0 mg, 0.104 mmol). The mixture was degassed 4 times with nitrogen and stirred at 110° C. for 2 hrs. The mixture was then cooled to 20° C., diluted with H₂O (10 mL), and extracted with EtOAc (3×15 mL). The combined organic layers were washed with brine, dried over Na₂SO₄ and concentrated under reduced pressure. The crude residue was purified by flash column chromatography (Silica gel, Biotage, EtOAc/Pet. ether 0-60%) to afford 147a as a yellow solid (700 mg, 79%). LCMS m/z 857.3 (M+H)⁺.

### Step 2: Synthesis of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)pyrrolidine-1-carboxylate (147b)

To a solution of tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-2,5-dihydro-1H-pyrrole-1-carboxylate (147a) (630 mg, 0.735 mmol) in EtOAc (10 mL) was added platinum dioxide (400 mg, 0.18 mmol). The resulting black mixture was stirred at 15° C. for 16 hrs under hydrogen. Additional platinum dioxide was added (400 mg, 0.18 mmol) and the mixture was stirred at 15° C. under hydrogen for another 16 hrs. At this point, a third portion of platinum dioxide was added (200 mg, 0.09 mmol) and the mixture was again stirred at 15° C. for 16 hrs under hydrogen. The mixture was filtered and the filter cake was washed with EtOAc (3×20 mL). The combined organic layers were concentrated under reduced pressure to afford 147b which was carried forward to the next step as is (570 mg, 95%). LCMS m/z 859.3 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-(pyrrolidin-3-yl)benzene-1-sulfonamide (147c)

To a solution of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)pyrrolidine-1-carboxylate (147b) (570 mg, 0.65 mmol) in DCM (3.0 mL) was added TFA (3.0 mL, 0.1 M). The mixture was stirred at 15° C. for 16 hrs, then concentrated under reduced pressure to afford 147c as a brown gum. Quantitative yield was assumed, and the crude material was carried forward asis (700 mg). LCMS m/z 555.2 (M+H)⁺.

### Step 4: Synthesis of N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide (Example 147) and N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide (Example 148)

To a suspension of N-{6-[(3-cyclopropyl-1H-pyrazol-5-yl)amino]-5-methoxy-1,2-benzoxazol-3-yl}-2,6-dimethoxy-4-(pyrrolidin-3-yl)benzene-1-sulfonamide (147c) (650 mg, 0.70 mmol) in MeOH (8.0 mL) was added formaldehyde (571 mg, 7.03 mmol) at 15° C. The mixture was stirred at 15° C. for 20 min. Sodium cyanoborohydride was added (663 mg, 10.5 mmol), and the mixture was stirred at 15° C. for 1 hr. The resulting mixture was concentrated under reduced pressure, and the crude residue was purified by preparative HPLC (C18 150×40 mm column, Mobile phase A: H₂O+NH₃H₂O+NH₄HCO₃ Mobile phase B: ACN 0-40% over 9 min Flow rate 60 mL/min) to afford a racemic mixture of N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide as a white solid (47.13 mg, 12%). LCMS m/z 569 (M+H)⁺; ¹H NMR (400 MHZ, DMSO-d₆) δ 11.92-11.87 (m, 1H), 11.13-10.52 (m, 1H), 8.13-8.09 (m, 1H), 8.02 (s, 1H), 7.40 (s, 1H), 6.64 (s, 2H), 5.79 (s, 1H), 3.87 (s, 3H), 3.76 (s, 6H), 2.84 (br. t, J=8.7 Hz, 1H), 2.73-2.58 (m, 3H), 2.33 (s, 3H), 2.25-2.19 (m, 1H), 1.92-1.74 (m, 3H), 0.95-0.90 (m, 2H), 0.69-0.64 (m, 2H). The enantiomers were separated by chiral SFC (30% methanol with 10 mm NH₃ in CO₂; 100 mL/min flow rate; 120 bar; ChromegaChiral CCO F4, 250 mm×20 mm, 5 μm).

### N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide (Example 147)

The first eluting peak was isolated as a white solid (10 mg, 25% yield). LCMS m/z 569.0 (M+H)⁺; ¹H NMR (600 MHZ, DMSO-d₆) δ 11.89 (br s, 1H), 8.11 (s, 1H), 8.01 (s, 1H), 7.40 (s, 1H), 6.66 (s, 2H), 5.78 (s, 1H), 3.87 (s, 3H), 3.76 (s, 6H), 2.26 (br s, 1H), 1.89-1.81 (m, 2H), 0.95-0.89 (m, 2H), 0.70-0.62 (m, 2H); [a]_{D}²²=−7.9° (c 0.1, MeOH).

### N-{6-[(3-cyclopropyl-1H-pyrazol-5-yl)amino]-5-methoxy-1,2-benzoxazol-3-yl}-2,6-dimethoxy-4-(1-methylpyrrolidin-3-yl)benzene-1-sulfonamide (Example 148)

The second eluting peak was isolated as a white solid (10 mg, 25% yield). LCMS m/z 569.0 (M+H)⁺; ¹H NMR (600 MHZ, DMSO-d₆) δ 11.89 (br s, 1H), 8.11 (s, 1H), 8.02 (s, 1H), 7.40 (s, 1H), 6.66 (s, 2H), 5.77 (s, 1H), 3.87 (s, 3H), 3.76 (s, 7H), 2.32-2.22 (m, 1H), 1.90-1.81 (m, 2H), 0.95-0.88 (m, 2H), 0.70-0.60 (m, 2H); [a]_{D}²²=+25.5° (c 0.1, MeOH).

### Examples 149 and 150: N-{6-[(3-cyclopropyl-1H-pyrazol-5-yl)amino]-5-methoxy-1,2-benzoxazol-3-yl}-2,6-dimethoxy-4-(1-methylpiperidin-3-yl)benzene-1-sulfonamide

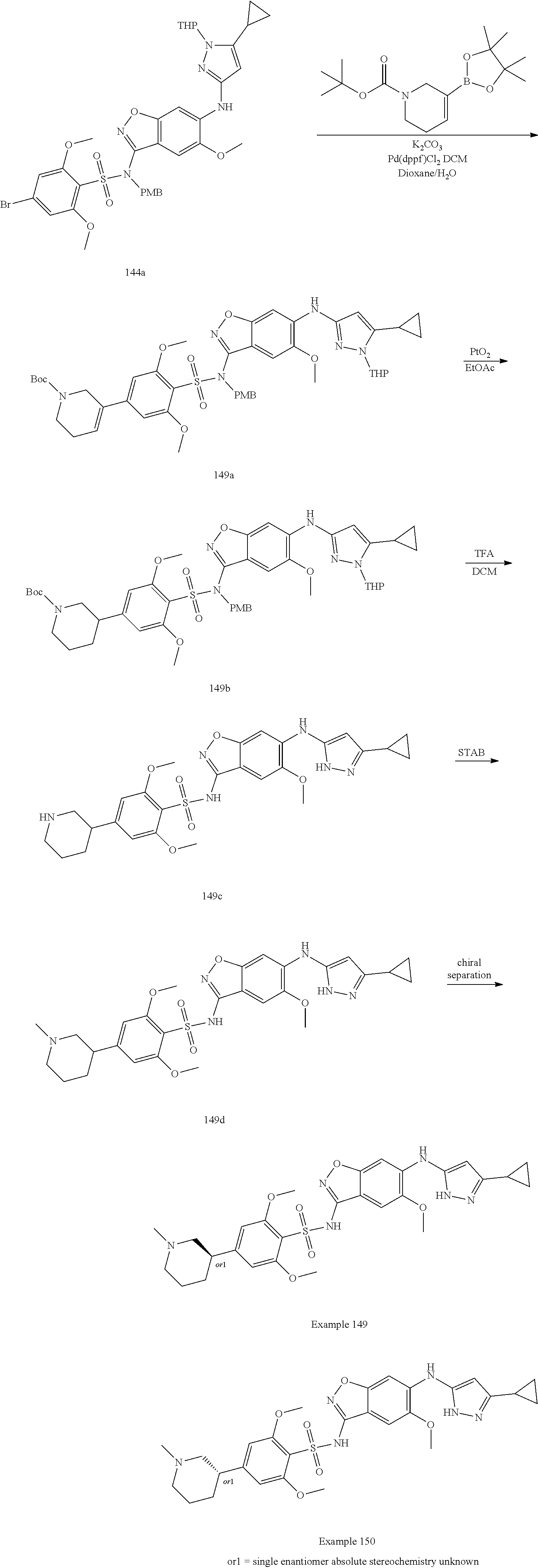

### Step 1: Synthesis of tert-butyl 5-{4-[{(6-{[5-cyclopropyl-1-(oxan-2-yl)-1H-pyrazol-3-yl]amino}-5-methoxy-1,2-benzoxazol-3-yl)[(4-methoxyphenyl)methyl]amino}(methylidene)oxo-λ⁶-sulfanyl]-3,5-dimethoxyphenyl}-3,6-dihydropyridine-1(2H)-carboxylate (149a)

tert-Butyl 5-{4-[{(6-{[5-cyclopropyl-1-(oxan-2-yl)-1H-pyrazol-3-yl]amino}-5-methoxy-1,2-benzoxazol-3-yl)[(4-methoxyphenyl)methyl]amino}(methylidene)oxo-λ⁶-sulfanyl]-3,5-dimethoxyphenyl}-3,6-dihydropyridine-1 (2H)-carboxylate (149a) was prepared in a similar manner as 147a using tert-butyl 5-(4,4,5,5-tetramethyl-1,3,2-dioxaborolan-2-yl)-3,6-dihydropyridine-1 (2H)-carboxylate in place of tert-butyl 3-(4,4,5,5-tetramethyl-1,3,2-dioxaborolan-2-yl)-2,5-dihydro-1H-pyrrole-1-carboxylate. The resulting crude was purified by flash silica gel column chromatography to afford 149a (470 mg, 83%) as a yellow solid. ¹H NMR (400 MHZ, CHLOROFORM-d) δ 7.89-7.82 (m, 1H), 7.40-7.35 (m, 2H), 7.27-7.23 (m, 1H), 7.01-6.94 (m, 1H), 6.79-6.71 (m, 2H), 6.57-6.49 (m, 2H), 6.32-6.26 (m, 1H), 5.67-5.64 (m, 1H), 5.53-5.44 (m, 1H), 5.00-4.97 (m, 2H), 4.80-4.79 (m, 2H), 4.30-4.18 (m, 2H), 4.14-4.08 (m, 1H), 3.92-3.90 (m, 3H), 3.75-3.73 (m, 3H), 3.72-3.70 (m, 6H), 3.60-3.54 (m, 2H), 2.62-2.50 (m, 1H), 2.40-2.31 (m, 2H), 2.20-2.11 (m, 1H), 2.03-1.94 (m, 1H), 1.92-1.83 (m, 1H), 1.81-1.69 (m, 2H), 1.53-1.51 (m, 9H), 1.02-0.95 (m, 2H), 0.85-0.77 (m, 1H), 0.69-0.61 (m, 1H); LCMS m/z 871.3 (M+H)⁺.

### Step 2: Synthesis of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl) piperidine-1-carboxylate (149b)

tert-Butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl) piperidine-1-carboxylate (149b) was prepared in a similar manner as 147b but using 149a in place of 147a. The resulting crude (470 mg, crude yield: ˜100%) was used in the next step without further purification, LCMS m/z 873.3 (M+H)⁺.

### Step 3: N-{6-[(5-cyclopropyl-1H-pyrazol-3-yl)amino]-5-methoxy-1,2-benzoxazol-3-yl}-2,6-dimethoxy-4-(piperidin-3-yl)benzene-1-sulfonamide (149c)

N-{6-[(5-cyclopropyl-1H-pyrazol-3-yl)amino]-5-methoxy-1,2-benzoxazol-3-yl}-2,6-dimethoxy-4-(piperidin-3-yl)benzene-1-sulfonamide (149c) was prepared in a similar manner as 147c but using 149b in place of 147b. The resulting crude material (750 mg, crude yield >99%) was used in the next step without further purification, LCMS m/z 569.2 (M+H)⁺. Step 4: N-{6-[(3-cyclopropyl-1H-pyrazol-5-yl)amino]-5-methoxy-1,2-benzoxazol-3-yl}-2,6-dimethoxy-4-(1-methylpiperidin-3-yl)benzene-1-sulfonamide (149d)

N-{6-[(3-cyclopropyl-1H-pyrazol-5-yl)amino]-5-methoxy-1,2-benzoxazol-3-yl}-2,6-dimethoxy-4-(1-methylpiperidin-3-yl)benzene-1-sulfonamide (149d) was prepared in a similar manner as 147d but using 149c in place of 147c. The resulting crude was purified by preparative HPLC (Xtimate C18 150×40 mm×5 mm Mobile phase A: Water (FA) Mobile phase B: ACN 5-45% over 9 mins, flow rate 60 mL/mins) to afford 149d (120.6 mg,) as a white solid. ¹H NMR (400 MHz, DMSO-d₆) δ 8.20-8.15 (m, 1H), 8.12-8.08 (m, 1H), 8.08-8.03 (m, 1H), 7.43-7.37 (m, 1H), 6.65-6.58 (m, 2H), 5.80-5.74 (m, 1H), 3.90-3.83 (m, 3H), 3.79-3.72 (m, 6H), 2.92-2.83 (m, 2H), 2.82-2.73 (m, 1H), 2.30-2.23 (m, 3H), 2.20-1.99 (m, 2H), 1.93-1.82 (m, 1H), 1.81-1.66 (m, 2H), 1.65-1.53 (m, 1H), 1.50-1.39 (m, 1H), 0.96-0.84 (m, 2H), 0.69-0.61 (m, 2H), LCMS m/z 583.2 (M+H)⁺. The enantiomers were separated by chiral SFC (30% methanol with 10 mM NH₃ in CO₂; 4.0 mL/min flow rate; 160 bar; ChromegaChiral CCO F4, 100 mm×4 mm, 3 μm).

### N-{6-[(3-cyclopropyl-1H-pyrazol-5-yl)amino]-5-methoxy-1,2-benzoxazol-3-yl}-2,6-dimethoxy-4-(1-methylpiperidin-3-yl)benzene-1-sulfonamide (Example 149)

The first eluting peak was isolated as a white solid (35 mg, 31% yield). LCMS m/z 583.2 (M+H)⁺; ¹H NMR (600 MHZ, DMSO-d₆) δ 8.16 (br s, 1H), 8.07 (br s, 1H), 8.01 (s, 1H), 7.39 (s, 1H), 6.62 (s, 2H), 5.77 (s, 1H), 3.86 (s, 3H), 3.75 (s, 6H), 2.89-2.74 (m, 3H), 2.24 (s, 3H), 2.13-1.99 (m, 2H), 1.88-1.82 (m, 1H), 1.80-1.66 (m, 2H), 1.63-1.54 (m, 1H), 1.49-1.41 (m, 1H), 0.94-0.88 (m, 2H), 0.67-0.63 (m, 2H); [a]_{D}²²=+6.9° (c 0.1, MeOH/DCM: CHCl₃ (6:2:2).

### N-{6-[(3-cyclopropyl-1H-pyrazol-5-yl)amino]-5-methoxy-1,2-benzoxazol-3-yl}-2,6-dimethoxy-4-(1-methylpiperidin-3-yl)benzene-1-sulfonamide (Example 150)

The second eluting peak was isolated as a white solid (37 mg, 32% yield). LCMS m/z 583.2 (M+H)⁺; ¹H NMR (600 MHZ, DMSO-d₆) δ 12.03-11.71 (m, 1H), 8.18-8.04 (m, 1H), 8.01 (s, 1H), 7.39 (s, 1H), 6.62 (s, 2H), 5.78 (s, 1H), 3.86 (s, 3H), 3.76 (s, 6H), 2.88-2.72 (m, 3H), 2.22 (s, 3H), 2.10-1.94 (m, 2H), 1.85 (tt, J=5.0, 8.4 Hz, 1H), 1.80-1.66 (m, 2H), 1.57 (tq, J=3.7, 12.5 Hz, 1H), 1.42 (dq, J=3.8, 12.4 Hz, 1H), 0.95-0.89 (m, 2H), 0.69-0.63 (m, 2H); [a]_{D}²²=−66.8° (c 0.1, MeOH/DCM: CHCl₃ (6:2:2).

### Example 151: N-{6-1 (3-cyclopropyl-1H-pyrazol-5-yl)amino]-5-methoxy-1,2-benzoxazol-3-yl}-2,6-dimethoxy-4-(1-methylpiperidin-4-yl)benzene-1-sulfonamide

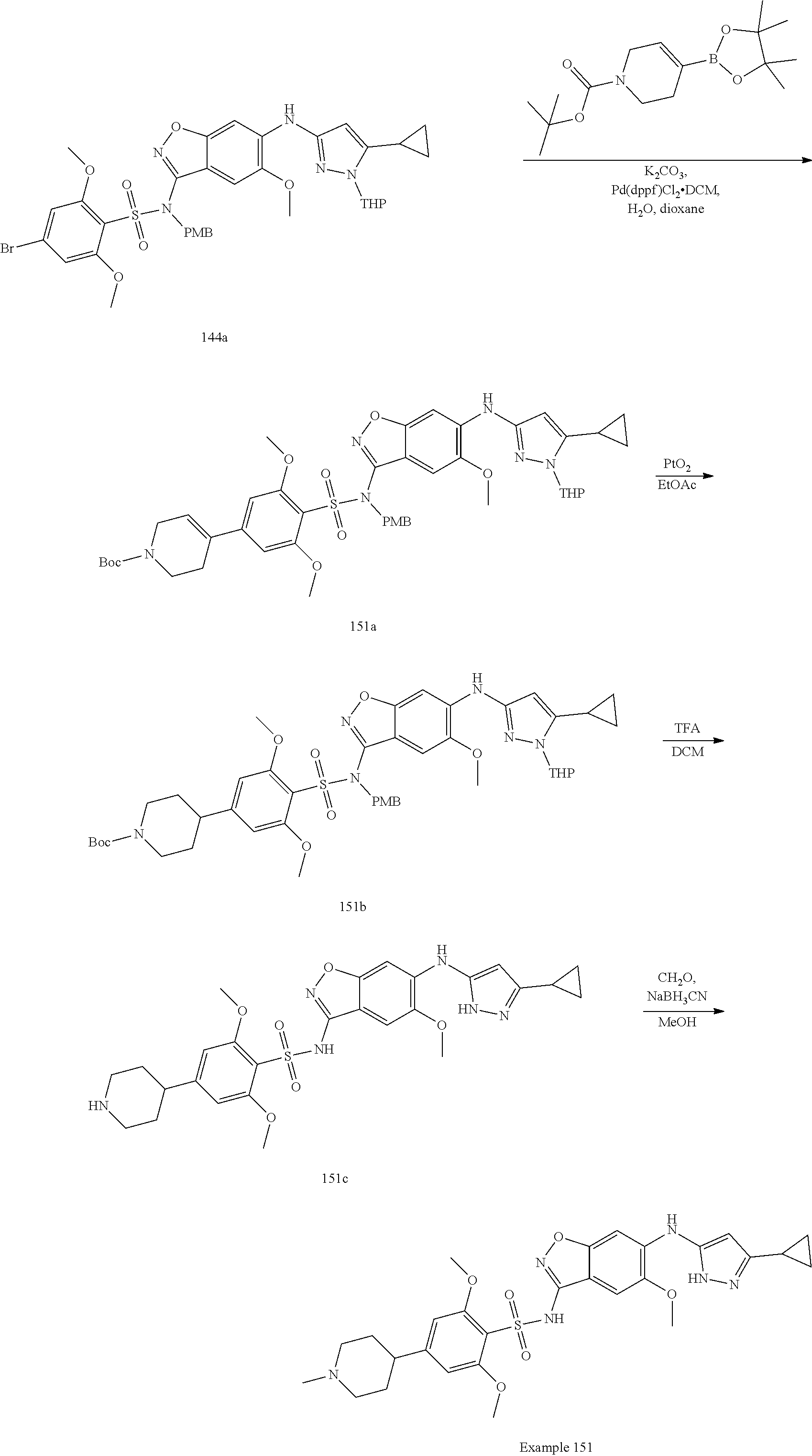

### Step 1: Synthesis of tert-butyl 4-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)-3,6-dihydropyridine-1 (2H)-carboxylate (151a)

151a was made in a similar manner as Example 147a using 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide 144a (400 mg, 0.52 mmol) and N-Boc-1,2,3,6-tetrahydropyridine-4-boronic acid pinacol ester (193 mg, 0.624 mmol). The resulting residue was purified by silica gel column chromatography (eluting with 0-45% EtOAc in Pet. ether) to give 151a (520 mg, 92%) as a yellow solid. LCMS m/z 871.1 (M+H)⁺.

### Step 2: Synthesis of tert-butyl 4-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)piperidine-1-carboxylate (151b)

151b was made in a similar manner as Example 147b using tert-butyl 4-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-3,6-dihydropyridine-1 (2H)-carboxylate 151a (520 mg, 0.597 mmol) and platinum dioxide (339 mg, 0.149 mmol). The resulting crude 151b (500 mg, crude yield: 96%) was obtained as a yellow solid, which was used in the next step without further purification. LCMS m/z 873.3 (M+H)⁺.

### Step 3: Synthesis of N-{6-[(5-cyclopropyl-1H-pyrazol-3-yl)amino]-5-methoxy-1,2-benzoxazol-3-yl}-2,6-dimethoxy-4-(piperidin-4-yl)benzene-1-sulfonamide (151c)

151c was made in a similar manner as Example 147c using tert-butyl 4-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl) piperidine-1-carboxylate 151b (500 mg, 0.573 mmol). The resulting crude 151c (500 mg, crude yield: 100%) was obtained as a black gum, which was used in the next step without further purification. LCMS m/z 569.1 (M+H)⁺.

### Step 4: Synthesis of N-{6-[(3-cyclopropyl-1H-pyrazol-5-yl)amino]-5-methoxy-1,2-benzoxazol-3-yl}-2,6-dimethoxy-4-(1-methylpiperidin-4-yl)benzene-1-sulfonamide (Example 151)

Example 151 was made in a similar manner as Example 147 using N-{6-[(5-cyclopropyl-1H-pyrazol-3-yl)amino]-5-methoxy-1,2-benzoxazol-3-yl}-2,6-dimethoxy-4-(piperidin-4-yl)benzene-1-sulfonamide (450 mg, 0.47 mmol). The resulting crude product was purified by preparative HPLC (C18 150×30 mm column, Mobile phase A: H₂O+NH₃H₂O+NH₄HCO₃ Mobile phase B: ACN 7-32% over 9 mins, Flow rate 30 mL/min) to afford 151 (82 mg, 30%) as a white solid. ¹H NMR (400 MHZ, DMSO-d₆) δ 11.94-11.87 (m, 1H), 8.12 (s, 1H), 8.03 (s, 1H), 7.42-7.38 (m, 1H), 6.61 (s, 2H), 5.78 (s, 1H), 3.87 (s, 3H), 3.79 (br. s, 1H), 3.76 (s, 6H), 2.87 (br. d, J=11.1 Hz, 1H), 2.47-2.32 (m, 4H), 2.20 (s, 3H), 2.01-1.92 (m, 2H), 1.89-1.83 (m, 1H), 1.72-1.70 (m, 2H), 0.95-0.89 (m, 2H), 0.69-0.64 (m, 2H); LCMS m/z 583.3 (M+H)⁺.

### Examples 152 and 153: N-{6-[(3-cyclopropyl-1H-pyrazol-5-yl)amino]-5-methoxy-1,2-benzoxazol-3-yl}-2,6-dimethoxy-4-(4-methylmorpholin-2-yl)benzene-1-sulfonamide

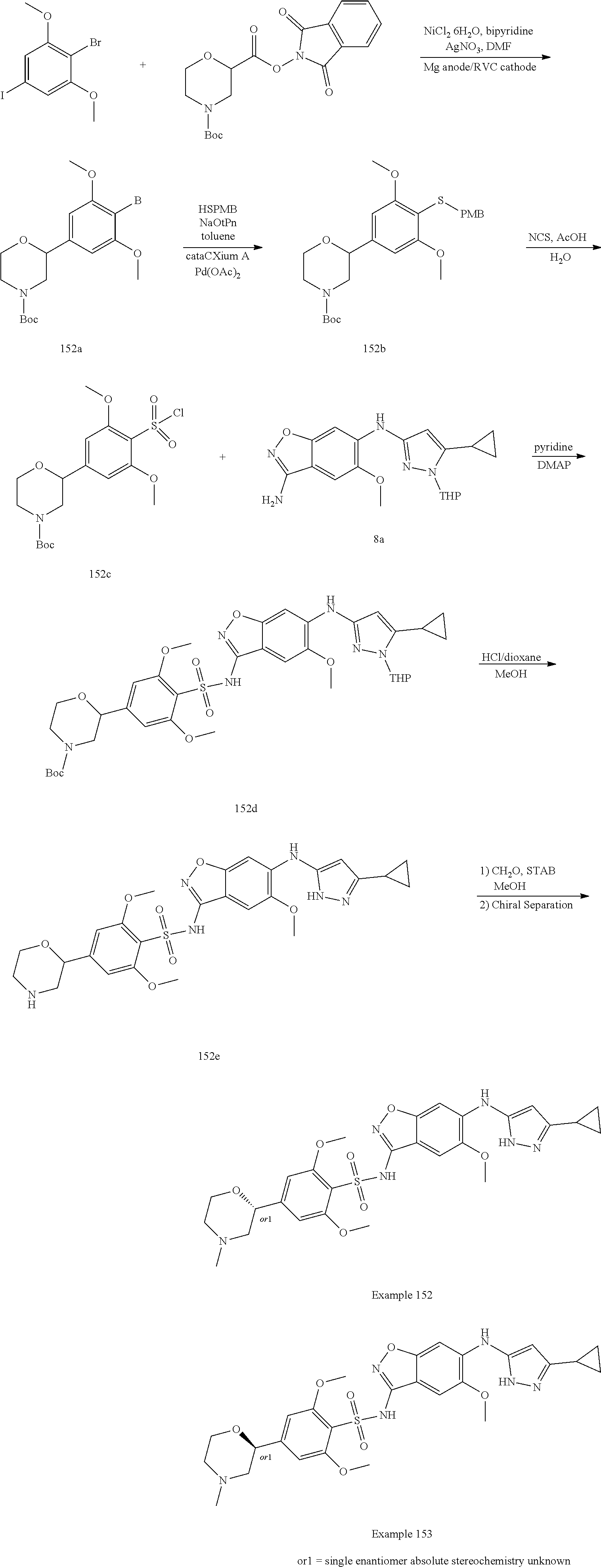

### Step 1: Synthesis of tert-butyl 2-(4-bromo-3,5-dimethoxyphenyl) morpholine-4-carboxylate (152a)

A vial was charged with 2-bromo-5-iodo-1,3-dimethoxybenzene (CAS #1318133-20-8) (1.03 g, 3.01 mmol), tert-butyl 2-{[(1,3-dioxo-1,3-dihydro-2H-isoindol-2-yl)oxy]carbonyl}morpholine-4-carboxylate (CAS #2240185-65-1) (755 mg, 2.01 mmol), nickel chloride hexahydrate (95.4 mg, 0.401 mmol), 2,2′-bipyridine (62.7 mg, 0.401 mmol), DMF (15.0 mL) and a stir bar. The mixture was stirred for about 5 mins, then silver nitrate (170 mg, 1.00 mmol) was added. The vial was closed with an IKA ElectraSyn 2.0 vial cap with a magnesium sacrificial anode (left side) and a reticulated vitreous carbon (RVC) cathode (right side), then the vial was immediately placed on an IKA ElectraSyn 2.0 stir plate. Electrolysis was set to 40 mA, 2.0 mmol, 4.0 F/mol. The mixture was allowed to stand for approx. 2.5 days. The resulting crude reaction mixture was added to 150 mL sat. aq NaHCO₃ and extracted with MTBE (3×50 mL). The combined organic layers were washed with brine, dried over Na₂SO₄, filtered, and concentrated under reduced pressure. To the crude residue was added approx. 10 mL of DCM. The mixture was filtered, and the filtrate was concentrated under reduced pressure to afford a white solid. This solid was dissolved in EtOAc and passed through a silica plug. The filtrate was concentrated under reduced pressure, and 3 mL of DCM was added to the resulting crude residue. Solids precipitated and were removed by filtration. The filtrate was purified over silica gel (2 mm chromatotron, EtOAc/heptanes 5-30%) to afford a white solid (390 mg). This material was combined with a separate batch made on a 150 mg scale in 4 mL of DCM (540 mg total). The resulting clear solution was again purified over silica gel (2 mm chromatotron) eluted with 0-5% EtOAc-heptane-DCM. The product was isolated as a clear oil which gradually dried to a white solid after standing to afford 152a (470 mg). LCMS m/z 302.5 (M+H−Boc)⁺.

### Step 2: Synthesis of tert-butyl 2-(3,5-dimethoxy-4-{[(4-methoxyphenyl)methyl]sulfanyl}phenyl) morpholine-4-carboxylate (152b)

To a flask was added tert-butyl 2-(4-bromo-3,5-dimethoxyphenyl) morpholine-4-carboxylate (152a) (425 mg, 1.06 mmol), toluene (10.6 mL), 4-methoxy-a-toluenethiol (196 mg, 1.27 mmol), sodium 2-methylbutan-2-olate (873 mg, 3.17 mmol, 40% by weight in toluene (0.95 mL)), and a mixture of cataCXium® A (23.7 mg, 0.066 mmol) and palladium (II) acetate (14.8 mg, 0.066 mmol) in toluene (1 mL). The flask was sealed with a rubber stopper, degassed and backfilled×3 with nitrogen, then placed in a pre-heated sand bath at 100° C. After approx. 20 hrs, the reaction mixture was cooled to rt and poured into 50 mL of 5% aq NaHCO₃. At this stage the material was combined with a previous test reaction performed on a 20 mg scale. To the mixture was added 50 mL of EtOAc. The layers were separated and the aqueous phase was extracted with EtOAc (2×50 mL). The organic layers were combined, washed with 50 mL of H₂O followed by 50 mL of brine, dried over Na₂SO₄, filtered, and concentrated under reduced pressure. The crude residue was purified by flash column chromatography (25 g silica gel, Biotage, EtOAc/heptane 5-40%) then concentrated under reduced pressure to afford 152b as an orange oil (437 mg, 87%). ¹H NMR (DMSO-d₆, 400 MHZ) δ 7.19-7.06 (m, 2H), 6.93-6.75 (m, 2H), 6.65 (s, 2H), 4.37 (dd, J=10.5, 2.5 Hz, 1H), 3.97 (dd, J=10.7, 2.2 Hz, 1H), 3.92 (s, 2H), 3.88 (br s, 1H), 3.81-3.78 (s, 7H), 3.70 (s, 3H), 3.55 (td, J=11.7, 2.8 Hz, 1H), 3.00 (br s, 1H), 2.81 (br s, 1H), 1.43 (s, 9H).

### Step 3: Synthesis of tert-butyl 2-[4-(chlorosulfonyl)-3,5-dimethoxyphenyl]morpholine-4-carboxylate (152c)

To a flask was added tert-butyl 2-(3,5-dimethoxy-4-{[(4-methoxyphenyl)methyl]sulfanyl}phenyl) morpholine-4-carboxylate (152b) (395 mg, 0.831 mmol) and acetic acid (6 mL). After an orange solution formed, H₂O (2 mL) was added, followed by NCS (333 mg, 2.49 mmol). After 15 min, the crude reaction mixture was added to MTBE (25 mL) and diluted with H₂O (60 mL). The layers were separated and the aqueous layer was extracted with MTBE (2×20 mL). The combined organic layers were washed with H₂O then brine, then dried over Na₂SO₄. The resulting material was filtered through a silica plug, which was washed with EtOAc (20 mL). The filtrate was concentrated under reduced pressure, then diluted with 1 mL of DCM and purified using a chromatotron (2 mm plate, eluted with 5-50% EtOAc/heptane) to afford 152c as a clear oil (244 mg, 70%). ¹H NMR (DMSO-d₆, 400 MHZ) δ 6.61 (s, 2H), 4.36 (dd, J=10.4, 2.4 Hz, 1H), 3.93-3.05 (m, 1H), 3.89 (br s, 1H), 3.80-3.76 (m, 1H), 3.71 (s, 6H), 3.59-3.48 (m, 1H), 3.02 (br s, 1H), 2.80 (br s, 1H), 1.43 (s, 9H).

### Step 4: Synthesis of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}morpholine-4-carboxylate (152d)

To a flask was added tert-butyl 2-[4-(chlorosulfonyl)-3,5-dimethoxyphenyl]morpholine-4-carboxylate (152c) (200 mg, 0.474 mmol), N6-[5-cyclopropyl-1-(oxan-2-yl)-1H-pyrazol-3-yl]-5-methoxy-1,2-benzoxazole-3,6-diamine (8a) (167 mg, 0.430 mmol), pyridine (2.2 mL) and DMAP in pyridine (7.88 mg, 0.0645 mmol, 0.79 mL of 10 mg/mL solution). The resulting solution was stirred at rt for 2.5 hrs, then diluted with EtOAc (20 mL) and aq HCl (20 mL, 1.2 M). The layers were separated, and the aqueous layer was extracted with EtOAc (20 mL). The combined organic layers were washed with aqueous HCl (1.2 M, 20 mL) and brine, dried over Na₂SO₄, and filtered. This material was combined with that of a test reaction performed on a 5 mg scale and concentrated under reduced pressure. The crude product was dissolved in DCM (1 mL) and purified over silica gel (2 mm chromatotron plate, eluted with 10-90% EtOAc/heptane+0.2-1% acetic acid). A portion of the material was re-purified using 50-80% EtOAc/heptane+1% AcOH. The pure fractions were combined to give 152d as a white solid (139 mg, 43%). LCMS m/z 755.3 (M+H)⁺; ¹H NMR (DMSO-d₆, 400 MHZ) δ 10.98 (s, 1H), 8.17 (s, 1H), 8.15 (s, 1H), 7.41 (s, 1H), 6.75 (s, 2H), 5.78 (s, 1H), 5.50 (dd, J=9.7, 2.4 Hz, 1H), 4.40 (dd, J=10.4, 2.6 Hz, 1H), 4.01-3.89 (m, 3H), 3.87 (s, 3H), 3.77 (s, 7H), 3.70-3.59 (m, 1H), 3.59-3.47 (m, 1H), 2.98 (br s, 1H), 2.79 (br s, 1H), 2.43-2.27 (m, 1H), 2.13-2.01 (m, 1H), 1.98-1.89 (m, 2H), 1.79-1.63 (m, 1H), 1.62-1.51 (m, 2H), 1.42 (s, 9H), 1.03-0.91 (m, 2H), 0.71-0.52 (m, 2H).

### Step 5: Synthesis of N-{6-[(3-cyclopropyl-1H-pyrazol-5-yl)amino]-5-methoxy-1,2-benzoxazol-3-yl}-2,6-dimethoxy-4-(morpholin-2-yl)benzene-1-sulfonamide (152e)

To a solution of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}morpholine-4-carboxylate (152d) (50 mg, 0.066 mmol) in MeOH (0.5 mL) was added HCl in dioxane (48 mg, 1.3 mmol, 4 M, 0.33 mL). The mixture was stirred at rt for approx. 20 hrs, at which point an additional 0.4 mL MeOH and 0.17 mL of 4 M HCl in dioxane were added. The resulting mixture was stirred at rt for 8 hrs, then concentrated under reduced pressure. MTBE was added and the mixture was again concentrated under reduced pressure to afford crude 152e as a white solid (54 mg, quant. yield). LCMS m/z 571.4 (M+H)⁺.

### Step 6: Synthesis of N-{6-[(3-cyclopropyl-1H-pyrazol-5-yl)amino]-5-methoxy-1,2-benzoxazol-3-yl}-2,6-dimethoxy-4-(4-methylmorpholin-2-yl)benzene-1-sulfonamide (Example 152) and N-{6-[(3-cyclopropyl-1H-pyrazol-5-yl)amino]-5-methoxy-1,2-benzoxazol-3-yl}-2,6-dimethoxy-4-(4-methylmorpholin-2-yl)benzene-1-sulfonamide (Example 153)

To a flask was added N-{6-[(3-cyclopropyl-1H-pyrazol-5-yl)amino]-5-methoxy-1,2-benzoxazol-3-yl}-2,6-dimethoxy-4-(morpholin-2-yl)benzene-1-sulfonamide (152e) (40 mg, 0.066 mmol) and MeOH (1.0 mL). Formaldehyde (20 mg, 0.66 mmol) and STAB (140 mg, 0.66 mmol) were added portionwise over 1 min. The resulting clear solution was stirred at rt for 1.5 hrs, then quenched with H₂O (0.1 mL). The material was purified by preparative chiral SFC (ChiralPak® IK 21×250 mm column, 5 μm particle size, eluted with 46% MeOH+10 mM NH₃ in CO₂, Flow rate 70 mL/min).

### N-{6-[(3-cyclopropyl-1H-pyrazol-5-yl)amino]-5-methoxy-1,2-benzoxazol-3-yl}-2,6-dimethoxy-4-(4-methylmorpholin-2-yl)benzene-1-sulfonamide (Example 152)

The first eluting peak was isolated as a white solid (13.3 mg, 35%). LCMS m/z 585.0 (M+H)⁺; ¹H NMR (DMSO-d₆, 400 MHZ) δ 11.89 (br. d, J=3.50 Hz, 1H), 10.92 (br. s, 1H), 8.12 (br. s, 1H), 8.02 (s, 1H), 7.40 (s, 1H), 6.71 (s, 2H), 5.79 (s, 1H), 4.48-4.47 (m, 1H), 3.98-3.92 (m, 1H), 3.87 (s, 3H), 3.76 (s, 6H), 3.71-3.60 (m, 1H), 3.02-2.89 (m, 1H), 2.69-2.66 (m, 1H), 2.21 (br. s, 3H), 2.10-2.02 (m, 1H), 1.94-1.76 (m, 2H), 0.94-0.90 (m, 2H), 0.69-0.65 (m, 2H); [a]_{D}²²=+3.9° (c 0.1, MeOH).

### N-{6-[(3-cyclopropyl-1H-pyrazol-5-yl)amino]-5-methoxy-1,2-benzoxazol-3-yl}-2,6-dimethoxy-4-(4-methylmorpholin-2-yl)benzene-1-sulfonamide (Example 153)

The second eluting peak was isolated as a white solid (13.0 mg, 34%). LCMS m/z 585.0 (M+H)⁺; ¹H NMR (DMSO-d₆, 400 MHZ) δ 11.89 (br. s, 1H) 10.92 (br. s, 1H), 8.11 (br. s, 1H), 8.00 (s, 1H), 7.38 (s, 1H), 6.70 (s, 2H), 5.78 (s, 1H), 4.46 (dd, J=10.1, 2.0 Hz, 1H), 3.94 (dd, J=11.3, 2.1 Hz, 1H), 3.87 (s, 3H), 3.75 (s, 6H), 3.69-3.59 (m, 1H), 2.92 (br. d, J=11.1 Hz, 1H), 2.71-2.60 (m, 1H), 2.20 (s, 3H), 2.05 (td, J=11.5, 3.1 Hz, 1H), 1.92-1.84 (m, 1H), 1.84-1.73 (m, 1H), 0.99-0.87 (m, 2H), 0.72-0.62 (m, 2H); [a]_{D}²²=−19.1° (c 0.1, MeOH).

### Example 154: N-{6-[(3-cyclopropyl-1H-pyrazol-5-yl)amino]-5-methoxy-1,2-benzoxazol-3-yl}-2,6-dimethoxy-4-(1-methylpyrrolidin-2-yl)benzene-1-sulfonamide

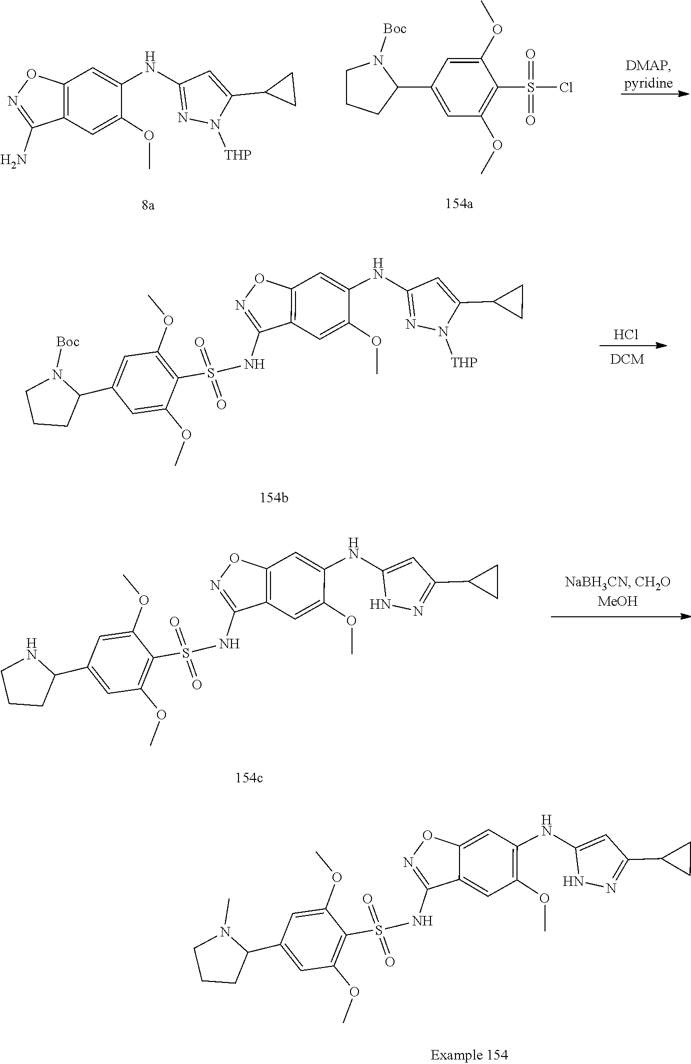

### Synthesis of tert-butyl 2-(4-(chlorosulfonyl)-3,5-dimethoxyphenyl)pyrrolidine-1-carboxylate 154a

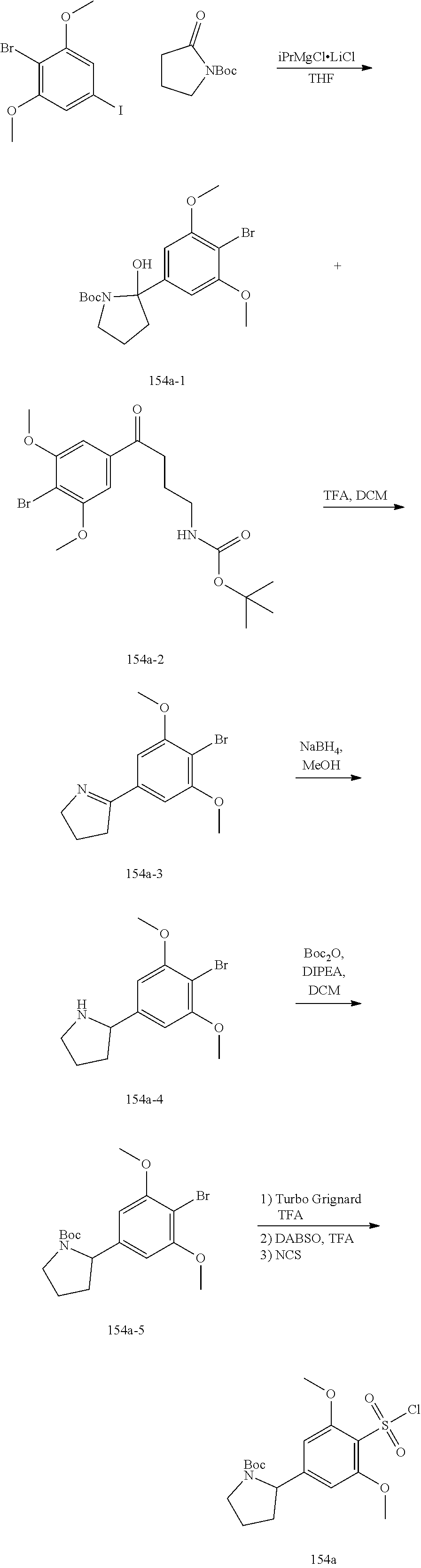

### Synthesis of tert-butyl 2-(4-bromo-3,5-dimethoxyphenyl)-2-hydroxypyrrolidine-1-carboxylate (154a-1)

To a solution of 2-bromo-5-iodo-1,3-dimethoxybenzene (5000.0 mg, 14.58 mmol) in THF (72.9 mL) was added 1.30 M isopropylmagnesium chloride lithium chloride complex solution (2330 mg, 16.0 mmol) at −70° C. After stirring for 1 hr, a solution of tert-butyl 2-oxopyrrolidine-1-carboxylate (3240 mg; 17.5 mmol) was added at −70° C. The reaction was stirred at −70° C. for 20 mins and stirred at r.t (˜20° C.) for 2 hrs. The mixture was quenched with sat. NH₄Cl (10 mL) and the aqueous layer was extracted with EtOAc (50 mL×3). The combined organic layer was washed with brine (50 mL), dried over Na₂SO₄, filtered and concentrated to afford a crude mixture of 154a-1 and 154a-2 (5.865 g) as a yellow oil, which was used in the next step without further purification.

### Synthesis of 5-(4-bromo-3,5-dimethoxyphenyl)-3,4-dihydro-2H-pyrrole (154a-3)

To a solution of crude 154a-1 and 154a-2 (5860.0 mg, 14.57 mmol) in DCM (146 mL) was added TFA (18300 mg, 160 mmol) at r.t (˜20° C.). The reaction mixture was stirred for 5 hrs and was then concentrated under reduced pressure to afford a crude brown oil (4140 mg), which was used in the next step without further purification.

### Synthesis of 2-(4-bromo-3,5-dimethoxyphenyl)pyrrolidine (154a-4)

To a solution of the crude brown oil from the previous step (4140 mg, 14.57 mmol) in MeOH (85.7 mL) was added sodium borohydride (730 mg, 19.3 mmol) at r.t (˜18° C.). The reaction mixture was stirred for 20 min. and was then quenched with NH₄Cl (12 mL). The aqueous layer was extracted, dried over Na₂SO₄, filtered and concentrated under reduced pressure to afford a crude brown solid (4169 mg), which was used in the next step without further purification.

### Synthesis of tert-butyl 2-(4-bromo-3,5-dimethoxyphenyl)pyrrolidine-1-carboxylate (154a-5)

To a mixture of the crude brown solid from the previous step (4169.0 mg, 14.57 mmol) and DCM (48.6 mL) was added DIPEA (6590 mg, 51.0 mmol) and di-tert-butyl dicarbonate (4770 mg, 21.9 mmol) in portions. After addition, the mixture was stirred at room temperature (20° C.) for 2 hrs. The mixture was washed with Sat.NaHCO₃ (20 mL×3). The organic layer was dried over Na₂SO₄, filtered and concentrated under reduced pressure. The resulting residue was purified by silica gel column chromatography (120 g silica gel, eluting with 0-20% EtOAc in Pet ether) to afford 154a-5 (2450 mg, 43.5%) as a yellow gum. ¹NMR (400 MHZ, CHLOROFORM-d) δ 6.39 (s, 2H), 4.90-4.70 (m, 1H), 3.88 (s, 6H), 3.70-3.50 (m, 2H), 2.38-2.27 (m, 1H), 2.00-1.76 (m, 3H), 1.47 (br s, 3H), 1.24 (br s, 6H),

### Synthesis of tert-butyl 2-(4-(chlorosulfonyl)-3,5-dimethoxyphenyl)pyrrolidine-1-carboxylate 154a

A mixture of 154a-5 (1250.0 mg, 3.236 mmol) in THF (16.2 mL) was degassed by bubbling argon through for 10 mins. 1.30 M Isopropylmagnesium chloride lithium chloride complex solution (1000 mg, 8 mmol) was then added and the mixture was heated to 60° C. and stirred under argon for 5 hrs. After cooling, the reaction mixture was transferred to an ACN/dry ice bath (˜−35° C.). To the mixture was added DABSO (1940 mg, 8.09 mmol) and the reaction was stirred overnight, during which time it warmed to rt (˜18° C.). NCS (1730 mg, 12.9 mmol) was then added and the reaction was stirred for 2 hrs at 40° C. The reaction was quenched with NH₄Cl and extracted with EtOAc (20 mL×3). The combined organic phase was washed with brine, dried over Na₂SO₄, filtered and concentrated under reduced pressure. The resulting residue was purified by flash silica gel column chromatography (12 g silica gel, eluting with 0-40% EOAc in Pet ether) to give 154a (865 mg, 65.9%) as a yellow oil. ¹H NMR (400 MHZ, CHLOROFORM-d) δ 6.45 (s, 2H), 4.90-4.70 (m, 1H), 3.96 (s, 6H), 3.70-3.58 (m, 2H), 2.42-2.32 (m, 1H), 1.95-1.78 (m, 3H), 1.48 (br s, 3H), 1.26 (br s, 6H)

### Step 1: tert-butyl 2-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)pyrrolidine-1-carboxylate (154b)

To a flask was added tert-butyl 2-(4-(chlorosulfonyl)-3,5-dimethoxyphenyl)pyrrolidine-1-carboxylate 154a (112 mg, 1.1 eq, 276 μmol), N6-[5-cyclopropyl-1-(oxan-2-yl)-1H-pyrazol-3-yl]-5-methoxy-1,2-benzoxazole-3,6-diamine (8a) (95 mg, 0.26 mmol), 4-dimethylaminopyridine (4.7 mg, 0.15 eq, 39 μmol) and pyridine (0.75 mL). The reaction was purged with nitrogen and stirred at rt for 2.5 hrs. The crude reaction was diluted with EtOAc (20 mL) and 1N HCl (15 mL). The layers were separated and the organic phase was washed with brine then dried (MgSO₄), filtered and concentrated. The crude residue was purified by column chromatography (eluting with 0-80% EtOAc with 1% AcOH in heptane) to give 154b (68 mg, 36%) as a yellow solid, LCMS m/z 739.2 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(pyrrolidin-2-yl)benzene-1-sulfonamide (154c)

To a flask containing 154b (68 mg, 1 eq, 92 μmol) was added DCM (1.0 mL). HCl (73 mg, 0.50 mL, 4 M in dioxane, 22 eq, 2.0 mmol) was added to the clear brown solution. Upon addition a precipitate formed that quickly dissolved to give clear orange solution. The reaction was stirred at rt. After about 15 min a white precipitate came out of solution. Stirring was continued for 3 hrs. A gummy residue formed at the bottom of the flask. 0.5 mL of MeOH were added. The reaction became homogeneous and was stirred for an additional 18 hrs. The crude reaction was concentrated under reduced pressure and dried on high vacuum to give crude 154c (58 mg) as a yellow solid which was used in the next step without further purification, ¹H NMR (400 MHz, DMSO-d₆) δ 11.09 (s, 1H), 10.13 (br s, 1H), 9.37 (br d, J=6.3 Hz, 1H), 8.28 (br d, J=1.9 Hz, 1H), 7.92 (s, 1H), 7.46 (s, 1H), 6.99 (s, 2H), 5.88 (s, 1H), 3.88 (s, 3H), 3.81 (s, 6H), 3.72-3.66 (m, 2H), 3.48 (br dd, J=4.3, 11.3 Hz, 2H), 2.41-2.28 (m, 1H), 2.04-1.95 (m, 2H), 1.93-1.85 (m, 1H), 1.01-0.91 (m, 2H), 0.78-0.67 (m, 2H), LCMS m/z 555.1 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-(1-methylpyrrolidin-2-yl)benzene-1-sulfonamide (154)

To a solution of 154c (58 mg, 1 Eq, 92 μmol) in methanol (1.0 mL) was added formaldehyde 37% solution in water (75 mg, 69 μL, 37% Wt, 10 Eq, 0.92 mmol) and STAB (50 mg, 3 Eq, 0.2 mmol). The reaction mixture was stirred at RT for 5 mins. The mixture was quenched with 0.5 mL of water, concentrated under reduced pressure and dried under high vacuum. The resulting orange residue was diluted in MeOH and DCM. The resulting solids were filtered off and the filtrate was concentrated. The resulting crude was purified as a racemate using preparative HPLC (Phenomenex Gemini NX-C18 150×21.2 mm column, 5 μm particle size, Mobile phase A: H₂O+10 mM ammonium acetate Mobile phase B: Acetonitrile 10-50% in 8 min, Flow rate 25 mL/min) to afford 154 (16.7 mg, 32%), LCMS m/z 569.2 (M+H)⁺; ¹H NMR (600 MHZ, DMSO-d₆) δ 11.89 (br. s, 1H), 10.89 (br. s, 1H), 8.11 (br. s, 1H), 8.01 (s, 1H), 7.39 (s, 1H), 6.69 (s, 2H), 5.78 (s, 1H), 3.87 (s, 3H), 3.75 (s, 6H), 3.16-3.11 (m, 1H), 3.10-3.05 (m, 1H), 2.23 (q, J=8.9 Hz, 1H), 2.15-2.07 (m, 4H), 1.86 (tt, J=5.0, 8.5 Hz, 1H), 1.82-1.77 (m, 1H), 1.76-1.69 (m, 1H), 1.56-1.50 (m, 1H), 0.96-0.87 (m, 2H), 0.70-0.62 (m, 2H).

### Example 155: N-{6-[(3-cyclopropyl-1H-pyrazol-5-yl)amino]-5-methoxy-1.2-benzoxazol-3-yl}-2,6-dimethoxy-4-(1-methylpiperidin-2-yl)benzene-1-sulfonamide

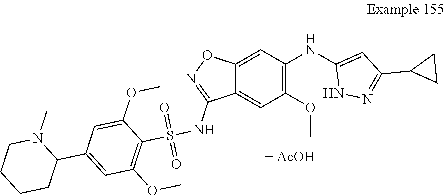 [SMILES: COc1cc2c(NS(=O)(=O)c3c(OC)cc(C4CCCCN4C)cc3OC)noc2cc1Nc1cc(C2CC2)n[nH]1]

Example 155 was made in a similar manner as Examples 152 and 153 using tert-butyl 2-{[(1,3-dioxo-1,3-dihydro-2H-isoindol-2-yl)oxy]carbonyl}piperidine-1-carboxylate in place of tert-butyl 2-{[(1,3-dioxo-1,3-dihydro-2H-isoindol-2-yl)oxy]carbonyl}morpholine-4-carboxylate in Step 1. Step 2 and Step 3 of Examples 152 and 153 were performed as a one-step sequence, wherein a solution of tert-butyl 2-(4-bromo-3,5-dimethoxyphenyl) piperidine-1-carboxylate (0.167 g, 417 μmol) in THF (2.0 mL) at rt was degassed by bubbling argon through the solution for 10 mins. An isopropylmagnesium chloride-lithium chloride complex (145 mg, 770 μL, 1.3 molar, 2.40 Eq, 1.00 mmol) was added and the reaction was heated to 60° C. under argon for 5 hrs, then transferred to an ACN/dry ice bath. 1,4-Diazabicyclo[2.2.2]octane bis (sulfur dioxide) adduct (251 mg, 1.04 mmol) was added and reaction was gradually warmed to rt and stirred for 18 hrs. To the reaction mixture was added NCS (223 mg, 1.67 mmol) and stirring was continued for 2 hrs at 40° C. The crude reaction was diluted with EtOAc (10 mL) and washed with water (10 mL) and brine (10 mL). The organic layer was concentrated and purified by flash column chromatography (Silica gel, 0-80% EtOAc/heptane) to give tert-butyl 2-[4-(chlorosulfonyl)-3,5-dimethoxyphenyl]piperidine-1-carboxylate as a clear oil (82 mg, 47%). For the final step, the material did not undergo separation by chiral SFC but was instead purified as a racemate using preparative HPLC (Phenomenex Gemini NX-C18 150×21.2 mm column, 5 μm particle size, Mobile phase A: H₂O+10 mM ammonium acetate Mobile phase B: Acetonitrile 10-50% in 12 min, Flow rate 40 mL/min). 155 was isolated as AcOH salt; LCMS m/z 583.9 (M+H)⁺; ¹H NMR (600 MHZ, DMSO-d₆) δ 8.02 (s, 1H), 7.89 (s, 1H), 7.31 (s, 1H), 6.63 (s, 2H), 5.76 (s, 1H), 3.84 (s, 3H), 3.70 (s, 6H), 2.91 (br. d, J=11.4 Hz, 1H), 2.75 (dd, J=2.4, 11.0 Hz, 1H), 2.00 (dt, J=2.6, 11.8 Hz, 1H), 1.90 (br. s, 3H), 1.89 (s, 3H), 1.85 (tt, J=5.1, 8.4 Hz, 1H), 1.70 (br. d, J=12.7 Hz, 1H), 1.64-1.51 (m, 3H), 1.43-1.34 (m, 1H), 1.26 (tq, J=3.6, 12.8 Hz, 1H), 0.94-0.89 (m, 2H), 0.68-0.64 (m, 2H).

### Example 156 and 157: N-{6-[(3-cyclopropyl-1H-pyrazol-5-yl)amino]-5-methoxy-1,2-benzoxazol-3-yl}-4-(4-ethylmorpholin-2-yl)-2,6-dimethoxybenzene-1-sulfonamide

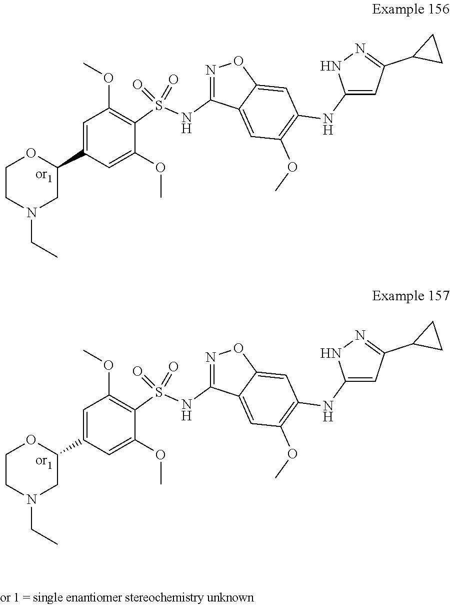 [SMILES: CCN1CCO[C@@H](c2cc(OC)c(S(=O)(=O)Nc3noc4cc(Nc5cc(C6CC6)n[nH]5)c(OC)cc34)c(OC)c2)C1 ; CCN1CCO[C@H](c2cc(OC)c(S(=O)(=O)Nc3noc4cc(Nc5cc(C6CC6)n[nH]5)c(OC)cc34)c(OC)c2)C1]

Examples 156 and 157 were made in a similar manner as Examples 152 and 153 using acetaldehyde in place of formaldehyde in the last step. The enantiomers were separated by chiral SFC (40% MeOH with 10 mM ammonium in CO₂; 70 mL/min flow rate, 120 bar, 40° C.; Chiralpak IK SFC, 250×21 mm, 5 μm).

### N-{6-[(3-cyclopropyl-1H-pyrazol-5-yl)amino]-5-methoxy-1,2-benzoxazol-3-yl}-4-(4-ethylmorpholin-2-yl)-2,6-dimethoxybenzene-1-sulfonamide (Example 156)

The first eluting peak was isolated as a white solid (6 mg, 22%, 99% ee). ¹H NMR (600 MHZ, DMSO-d₆) δ 11.91 (br. s, 1H), 8.11-8.06 (m, 1H), 8.05-8.01 (m, 1H), 7.38-7.32 (m, 1H), 6.70 (s, 2H), 5.76 (s, 1H), 4.50 (br. d, J=9.9 Hz, 1H), 3.98 (br. d, J=10.9 Hz, 1H), 3.88-3.83 (m, 3H), 3.75 (s, 6H), 1.88-1.80 (m, 1H), 1.04 (br. t, J=6.9 Hz, 3H), 0.94-0.88 (m, 2H), 0.67-0.61 (m, 2H) Additional protons under solvent peaks or very broad; LCMS m/z 599.2 (M+H)⁺;

### N-{6-[(3-cyclopropyl-1H-pyrazol-5-yl)amino]-5-methoxy-1,2-benzoxazol-3-yl}-4-(4-ethylmorpholin-2-yl)-2,6-dimethoxybenzene-1-sulfonamide (Example 157)

The second eluting peak was isolated as a white solid (6 mg, 22%, 92% ee). ¹H NMR (600 MHz, DMSO-d₆) δ 11.91 (br. s, 1H), 8.08 (br. s, 1H), 8.05 (s, 1H), 7.36 (s, 1H), 6.71 (s, 2H), 5.76 (s, 1H), 4.54 (br. d, J=7.0 Hz, 1H), 4.02 (br. d, J=9.5 Hz, 1H), 3.86 (s, 3H), 3.76 (s, 6H), 1.88-1.81 (m, 1H), 1.07 (br. s, 3H), 0.95-0.89 (m, 2H), 0.68-0.61 (m, 2H) Additional protons under solvent peaks or very broad; LCMS m/z 599.2 (M+H)⁺.

### Example 158 and 159: N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]piperidin-3-yl}benzene-1-sulfonamide

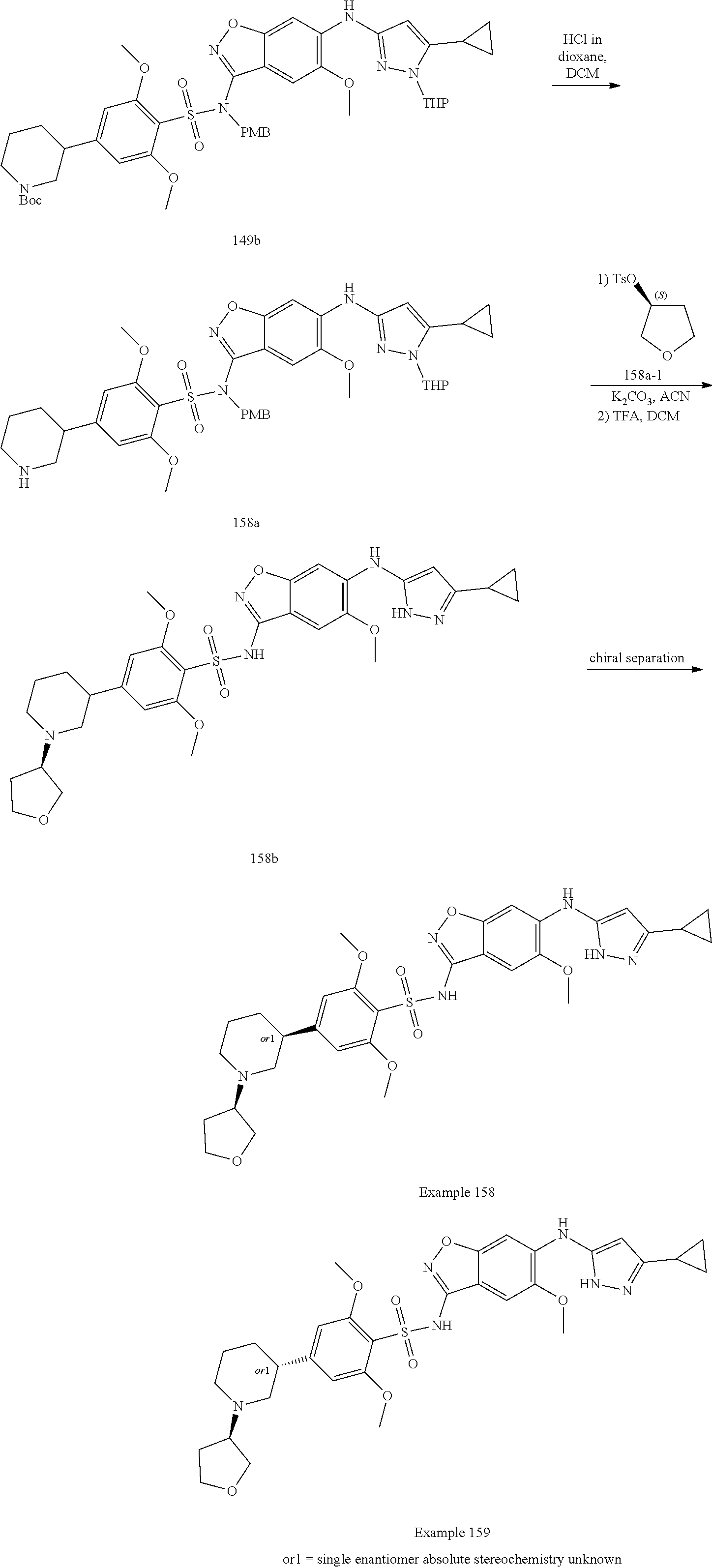

### Synthesis of (S)-tetrahydrofuran-3-yl 4-methylbenzenesulfonate 158a-1

To a white suspension of (S)-(+)-3-hydroxytetrahydrofuran (100 mg, 1.14 mmol) in DCM (2_mL) was added TEA (230 mg, 2.27 mmol) and tosyl chloride (260 mg, 1.36 mmol). The reaction mixture was stirred at 15-20° C. for 16 hrs. Water (10 mL) was added and the aqueous layer was extracted with EtOAc (10 mL×4). The combined organic phase was washed with brine (10 mL), dried over Na₂SO₄, filtered and concentrated under reduced pressure. The resulting residue was purified by flash silica gel column chromatography (eluting with 17-22% EtOAc in Per ether) to afford (S)-tetrahydrofuran-3-yl 4-methylbenzenesulfonate 158a-1 (200 mg, yield: 72.7%) as clean oil. ¹H NMR (400 MHZ, DMSO-d₆) δ 1.80-1.94 (m, 1H) 2.02-2.13 (m, 1H) 2.43 (s, 3H) 3.63-3.79 (m, 4H) 5.12 (td, J=3.96, 1.98 Hz, 1H) 7.43-7.55 (m, 2H) 7.81 (d, J=8.14 Hz, 2H)

### Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(piperidin-3-yl)benzene-1-sulfonamide 158a

A solution of tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl) piperidine-1-carboxylate 149b (2400 mg, 2.749 mmol) in 4M HCl in dioxane (10 mL) and DCM (10 mL) was stirred at 20° C. for 16 hrs. The reaction was concentrated to give crude 158a (2100 mg, 98%) which was a yellow solid and was used in the next step without further purification, LCMS m/z 773.3 (M+H)⁺.

### Synthesis of N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]piperidin-3-yl}benzene-1-sulfonamide (158b)

To a solution of 158a (500 mg, 0.725 mmol) in ACN (5 mL) was added K₂CO₃ (401 mg, 2.90 mmol) and(S)-tetrahydrofuran-3-yl 4-methylbenzenesulfonate 158-1 (527 mg, 2.17 mmol). The mixture was stirred at 100° C. for 16 hrs. The mixture was cooled to 30° C., then concentrated under reduced pressure to remove most of the solvent. The residue was diluted with EtOAc (10 mL), washed with H₂O (10 mL) and brine (10 mL), then dried over MgSO₄, filtered, and concentrated to afford a crude yellow oil, which was used in the next step without further purification.

To a solution of the crude material (600 mg) in DCM (5 mL) was added TFA (5 mL). The mixture was stirred at 20° C. for 16 hrs then concentrated under reduced pressure and diluted with water. Ammonium hydroxide (0.1 mL) was added and the mixture was extracted with EtOAc (2×2 mL). The combined extracts were washed with H₂O (2 mL), sat. aq. NaHCO₃ (2 mL) and brine (2 mL), then dried over Na₂SO₄, filtered, and concentrated. The crude residue was purified by preparative HPLC (Phenomenex Gemini NX 150×30 mm, 5 μm, Mobile phase A: H₂O+NH₃H₂O+NH₄HCO₃ Mobile phase B: ACN 6-46% over 9 min, Flow rate 60 mL/min) to afford a racemic mixture 158b as a white solid (260 mg, 52%). LCMS m/z 639.2 (M+H)⁺. The enantiomers were separated by chiral SFC (45% iPrOH with 0.1% NH₄OH in CO₂; 80 mL/min flow rate; Diacel Chiralcel OJ (250 mm×30 mm, 10 mm) to give x (60 mg, 23%) as white solid which was further purified by chiral SFC (50%/PrOH (0.1% NH₄OH) in CO₂; 150 mL/min flow rate; Diacel Chiralpak AD (250 mm×30 mm, 10 mm).

### N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]piperidin-3-yl}benzene-1-sulfonamide (Example 158)

The first eluting peak was isolated as a white solid (10 mg, 4%). LCMS m/z 639.2 (M+H)⁺; ¹H NMR (400 MHZ, DMSO-d₆) δ 11.91 (br. s, 1H), 11.26-10.50 (m, 1H), 8.41-7.93 (m, 2H), 7.39 (s, 1H), 6.80-6.52 (m, 2H), 5.78 (s, 1H), 3.86 (s, 3H), 3.75 (s, 6H), 3.65-3.54 (m, 3H), 2.92-2.82 (m, 2H), 2.68 (br. d, J=7.9 Hz, 2H), 2.13-2.02 (m, 2H), 2.01-1.90 (m, 2H), 1.85 (dt, J=4.6, 8.6 Hz, 1H), 1.77-1.63 (m, 3H), 1.55-1.44 (m, 2H), 0.91 (br. d, J=7.0 Hz, 2H), 0.69-0.60 (m, 2H); [a]_{D}²²=−23.9° (c 0.1, CH₃OH).

### N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]piperidin-3-yl}benzene-1-sulfonamide (Example 159)

The second eluting peak was isolated as a white solid (9 mg, 3%). LCMS m/z 639.2 (M+H)⁺; ¹H NMR (400 MHZ, DMSO-d₆) δ 11.91 (br. s, 1H), 11.24-10.64 (m, 1H), 8.28-7.93 (m, 2H), 7.40 (s, 1H), 6.64 (s, 2H), 5.77 (s, 1H), 3.86 (s, 3H), 3.76 (s, 6H), 3.60 (br. d, J=8.0 Hz, 3H), 2.93-2.86 (m, 2H), 2.75-2.69 (m, 2H), 2.21-2.06 (m, 2H), 2.02-1.93 (m, 2H), 1.85 (br. dd, J=3.9, 8.7 Hz, 1H), 1.80-1.67 (m, 3H), 1.56-1.44 (m, 2H), 0.91 (br. d, J=7.6 Hz, 2H), 0.69-0.60 (m, 2H); [a]_{D}²²=+16.908° (c 0.1, CH₃OH).

### Example 160 and 161: N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]pyrrolidin-3-yl}benzene-1-sulfonamide

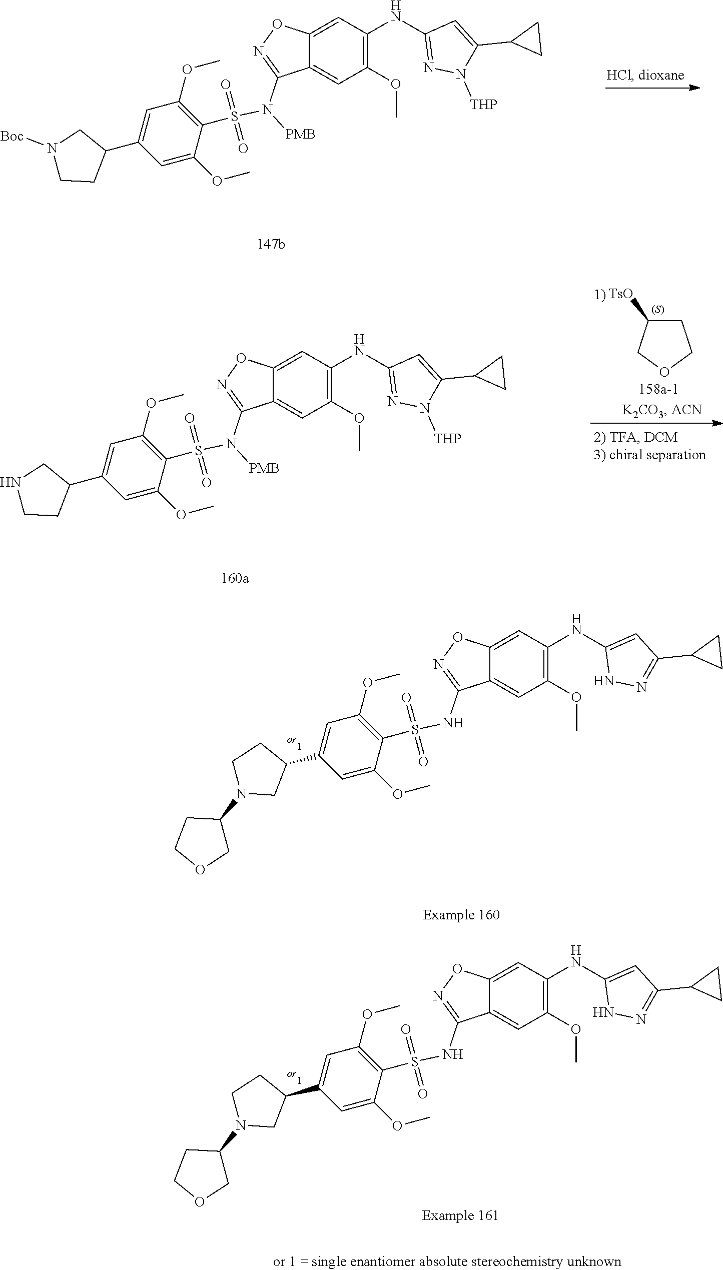

### Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(pyrrolidin-3-yl)benzene-1-sulfonamide (160a)

To a solution of 147b (1080 mg, 1.257 mmol) in DCM (4 mL) was added 4M HCl in dioxane (4 mL). The reaction mixture was stirred at 20° C. for 3 hrs and was then concentrated under reduced pressure to give crude 160a (1020 mg) which was a yellow solid and was used in the next step without further purification

### Synthesis of N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]pyrrolidin-3-yl}benzene-1-sulfonamide (Examples 160 and 161)

Examples 160 and 161 were made in a similar manner as Examples 158 and 159 using Intermediate 160a in place of 158a The racemic mixture was purified first by chiral SFC (55% MeOH with 0.1% NH₄OH in CO₂; 140 mL/min flow rate; Chiralpak IH, 250×30 mm, 10 μm) then the enantiomers were separated by chiral SFC (55% MeOH with 0.1% NH₄OH in CO₂; 80 mL/min flow rate, Daicel Chiralcel OJ, 250×30 mm, 10 μm).

### N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]pyrrolidin-3-yl}benzene-1-sulfonamide (Example 160)

The first eluting peak was isolated as a white solid (9 mg, 10%). ¹H NMR (400 MHZ, DMSO-d₆) δ 11.90 (br. s, 1H), 11.45-10.48 (m, 1H), 8.34-7.90 (m, 2H), 7.59-7.32 (m, 1H), 6.85-6.47 (m, 2H), 5.89-5.67 (m, 1H), 3.89-3.84 (m, 3H), 3.78 (s, 1H), 3.77-3.73 (m, 6H), 3.68 (s, 1H), 3.67-3.61 (m, 1H), 3.51 (dd, J=8.56, 5.87 Hz, 1H), 3.29-3.19 (m, 2H), 2.98-2.81 (m, 2H), 2.74-2.58 (m, 2H), 2.25-2.10 (m, 1H), 2.01-1.69 (m, 4H), 0.98-0.82 (m, 2H), 0.71-0.60 (m, 2H); LCMS m/z 625.2 (M+H)⁺; [a]_{D}²⁵=−23.936° (c 0.1, CH₃OH).

### N-{6-[(3-cyclopropyl-1H-pyrazol-5-yl)amino]-5-methoxy-1,2-benzoxazol-3-yl}-2,6-dimethoxy-4-{1-[(3R)-oxolan-3-yl]pyrrolidin-3-yl}benzene-1-sulfonamide (Example 161)

The second eluting peak was isolated as a white solid (15 mg, 17%). ¹H NMR (400 MHZ, DMSO-d₆) δ 11.90 (br. s, 1H), 11.44-10.68 (m, 1H), 8.25-7.94 (m, 2H), 7.40 (s, 1H), 6.77-6.57 (m, 2H), 5.88-5.69 (m, 1H), 3.90-3.83 (m, 3H), 3.80-3.76 (m, 1H), 3.76-3.73 (m, 6H), 3.73-3.70 (m, 1H), 3.68 (br. s, 2H), 3.54-3.49 (m, 1H), 3.27-3.23 (m, 1H), 2.94-2.77 (m, 2H), 2.71-2.59 (m, 2H), 2.25-2.09 (m, 1H), 2.02-1.63 (m, 4H), 0.94-0.86 (m, 2H), 0.69-0.62 (m, 2H); LCMS m/z 625.2 (M+H)⁺; [a]_{D}²⁵=+10.583° (c 0.1, CH₃OH).

### Example 162 and 163: N-{6-[(3-cyclopropyl-1H-pyrazol-5-yl)amino]-5-methoxy-1,2-benzoxazol-3-yl}-2,6-dimethoxy-4-{1-[(3S)-oxolan-3-yl]pyrrolidin-3-yl}benzene-1-sulfonamide

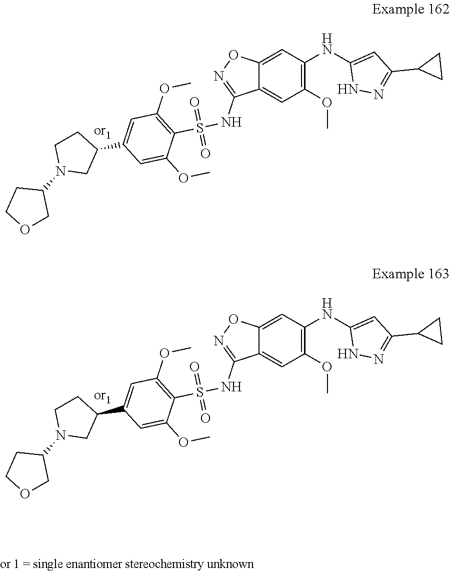

### Synthesis of (R)-tetrahydrofuran-3-yl 4-methylbenzenesulfonate 162a

(R)-tetrahydrofuran-3-yl 4-methylbenzenesulfonate 162a was prepared in a similar manner as(S) -tetrahydrofuran-3-yl 4-methylbenzenesulfonate 158a-1 using (R)-(−)-3-hydroxytetrahydrofuran in place of(S)-(+)-3-hydroxytetrahydrofuran

Examples 162 and 163 were made in a similar manner as Examples 160 and 161 using (R)-tetrahydrofuran-3-yl 4-methylbenzenesulfonate 162a in place of(S)-tetrahydrofuran-3-yl 4-methylbenzenesulfonate 158a-1. The enantiomers were separated by chiral SFC (55% i-PrOH with 0.1% NH₄OH in CO₂; 140 mL/min flow rate, ChiralPak IH, 250×30 mm, 10 μm).

### N-{6-[(3-cyclopropyl-1H-pyrazol-5-yl)amino]-5-methoxy-1,2-benzoxazol-3-yl}-2,6-dimethoxy-4-{1-[(3S)-oxolan-3-yl]pyrrolidin-3-yl}benzene-1-sulfonamide (Example 162)

The first eluting peak was isolated as a white solid (26 mg, 20%). ¹H NMR (400 MHZ, DMSO-d₆) δ 11.90 (br. s, 1H) 10.92 (br. s, 1H) 8.12 (s, 1H) 8.05 (s, 1H) 7.40 (s, 1H) 6.65 (s, 2H) 5.78 (s, 1H) 3.86 (s, 3H) 3.77 (br. s, 1H) 3.75 (s, 6H) 3.73-3.69 (m, 1H) 3.65 (q, J=7.25 Hz, 1H) 3.51 (dd, J=8.44, 5.94 Hz, 1H) 3.27-3.20 (m, 2H) 2.91-2.79 (m, 2H) 2.70-2.58 (m, 2H) 2.24-2.11 (m, 1H) 1.98-1.88 (m, 1H) 1.88-1.69 (m, 3H) 0.95-0.88 (m, 2H) 0.70-0.61 (m, 2H); LCMS m/z 625.2 (M+H)⁺; [a]_{D}²⁵=−7° (c 0.2, CH₃OH).

### N-{6-[(3-cyclopropyl-1H-pyrazol-5-yl)amino]-5-methoxy-1,2-benzoxazol-3-yl}-2,6-dimethoxy-4-{1-[(3S)-oxolan-3-yl]pyrrolidin-3-yl}benzene-1-sulfonamide (Example 163)

The second eluting peak was isolated as a white solid (28 mg, 22%). ¹H NMR (400 MHZ, DMSO-d₆) δ 11.91 (br. s, 1H) 11.14-10.69 (m, 1H) 8.13 (s, 1H) 8.07 (s, 1H) 7.41 (s, 1H) 6.67 (s, 2H) 5.79 (s, 1H) 3.87 (s, 3H) 3.82-3.77 (m, 1H) 3.76 (s, 6H) 3.74-3.70 (m, 1H) 3.66 (q, J=7.55 Hz, 1H) 3.52 (dd, J=8.44, 5.94 Hz, 1H) 3.30-3.22 (m, 2H) 2.99-2.84 (m, 2H) 2.77-2.60 (m, 2H) 2.24-2.13 (m, 1H) 2.03-1.93 (m, 1H) 1.92-1.73 (m, 3H) 0.94-0.88 (m, 2H) 0.69-0.64 (m, 2H); LCMS m/z 625.2 (M+H)⁺; [a]_{D}²⁵=+2° (c 0.2, CH₃OH).

### Example 164: N-{6-[(3-cyclopropyl-1H-pyrazol-5-yl)amino]-5-methoxy-1,2-benzoxazol-3-yl}-2,6-dimethoxy-4-[1-(oxetan-3-yl) piperidin-4-yl]benzene-1-sulfonamide

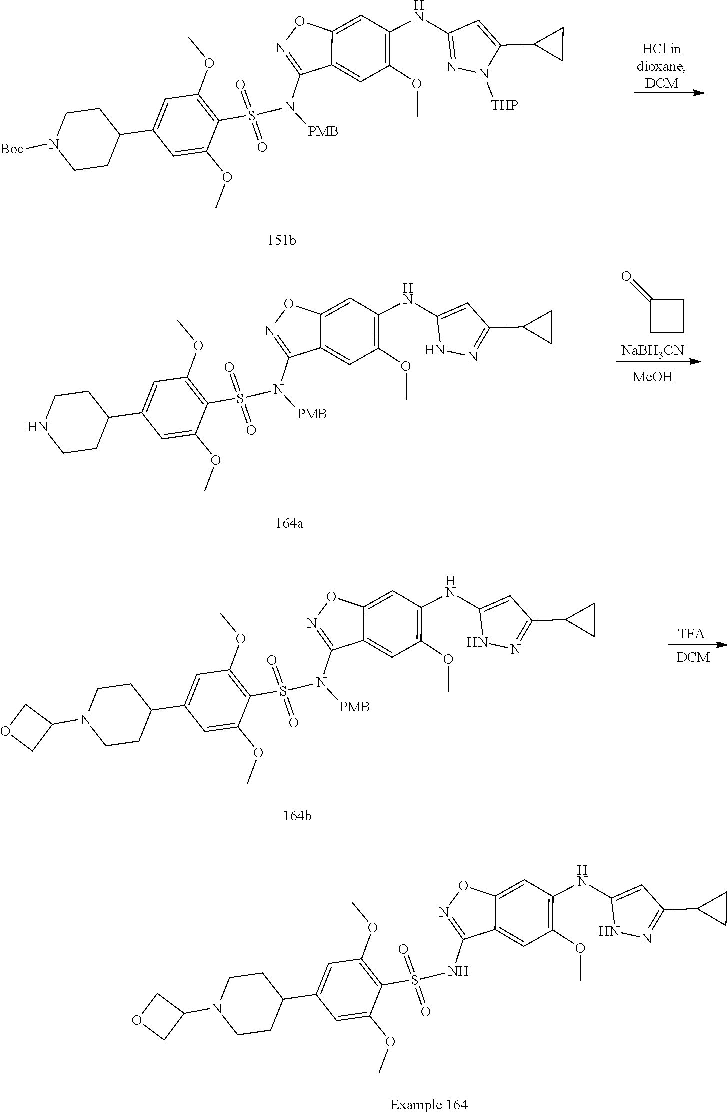

### Step 1: Synthesis of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-(piperidin-4-yl)benzene-1-sulfonamide 164a

A solution of 151b (1000 mg, 1.145 mmol) in DCM (4 mL) and 4M HCl in dioxane (4.0 mL) was stirred at 25° C. for 2 hrs. The reaction mixture was then concentrated under reduced pressure to give crude 164a (1000 mg, crude yield >99%) which was a yellow solid and was used in the next step without further purification, LCMS m/z 689.2 (M+H)⁺.

### Step 2: Synthesis of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[1-(oxetan-3-yl) piperidin-4-yl]benzene-1-sulfonamide (164b)

To a solution of N-{6-[(5-cyclopropyl-1H-pyrazol-3-yl)amino]-5-methoxy-1,2-benzoxazol-3-yl}-2,6-dimethoxy-N-[(4-methoxyphenyl)methyl]-4-(piperidin-4-yl)benzene-1-sulfonamide 164a (150 mg, 0.194 mmol) in MeOH (1.5 mL) was added 3-oxetanone (140 mg, 1.94 mmol) and sodium cyanoborohydride (61 mg, 0.97 mmol). The reaction mixture was stirred at ˜20° C. for 2 hrs and then combined with a smaller batch (50 mg of 164b). The resulting mixture was concentrated under reduced pressure to give crude 164b, which was used in the next step without further purification. LCMS m/z 745.3 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-[1-(oxetan-3-yl) piperidin-4-yl]benzene-1-sulfonamide (Example 164)

To a solution of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[1-(oxetan-3-yl) piperidin-4-yl]benzene-1-sulfonamide 164b (160 mg, 0.215 mmol) in DCM (2 mL) was added TFA (2 mL). The reaction mixture was stirred at 20° C. for 16 hrs and was then concentrated under reduced pressure. The resulting residue was diluted with ACN (3 mL) and purified by preparative HPLC (C18 150×30 mm column, Mobile phase A: water+NH₃H₂O+NH₄HCO₃, Mobile phase B: ACN 5-45% over 9 mins, flow rate 30 mL/min) to afford Example 164 as a white solid (44.3 mg, 33% yield); 1H NMR (400 MHZ, DMSO-d₆) δ 11.90 (br. s, 1H), 10.93-10.86 (m, 1H), 8.13 (s, 1H), 8.06 (s, 1H), 7.41 (s, 1H), 6.63 (s, 2H), 5.78 (s, 1H), 4.56-4.50 (m, 2H), 4.44-4.39 (m, 2H), 3.87 (s, 3H), 3.76 (s, 6H), 3.40-3.35 (m, 1H), 2.81-2.74 (m, 2H), 2.45-2.42 (m, 1H), 1.86 (s, 3H), 1.74-1.68 (m, 4H), 0.94-0.89 (m, 2H), 0.68-0.63 (m, 2H); LCMS m/z 625.2 (M+H)⁺.

### Example 165 and 166: N-{6-[(3-cyclopropyl-1H-pyrazol-5-yl)amino]-5-methoxy-1,2-benzoxazol-3-yl}-4-[1-(2,2-difluoroethyl)pyrrolidin-2-yl]-2,6-dimethoxybenzene-1-sulfonamide

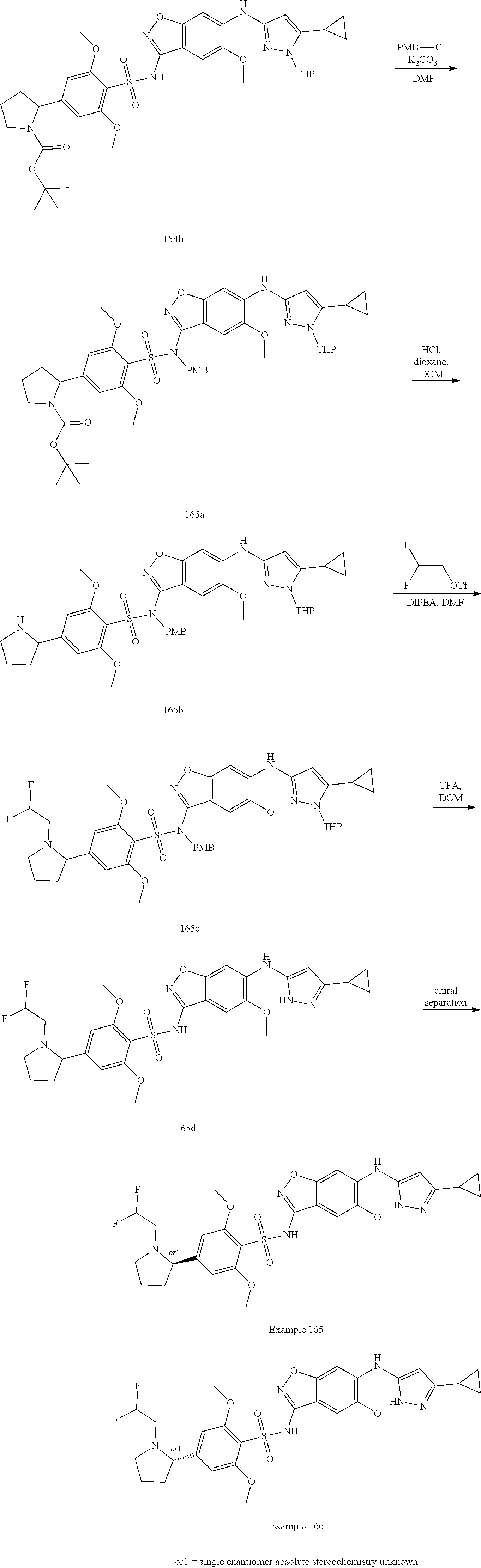

### Step 1: tert-butyl 2-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl]-3,5-dimethoxyphenyl)pyrrolidine-1-carboxylate (165a)

To a solution of 154b (820.0 mg 0.78 mmol), K₂CO₃ (236 mg, 1.71 mmol) in DMF (5.18 mL) was added 4-methoxybenzyl chloride (158 mg, 1.01 mmol). The reaction mixture was stirred at 80° C. for ˜3 hrs. The mixture was poured into water (˜20 mL) and extracted with ethyl acetate (20 mL×3), dried over Na₂SO₄, concentrated and purified by flash silica gel column chromatography (eluting with 0-80% EtOAc in Pet ether) to afford 165a (620 mg, 93%) as a yellow oil, LCMS m/z 859.4 (M+H)⁺.

### Step 2: N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(pyrrolidin-2-yl)benzene-1-sulfonamide (165b)

A solution of 165a (420.0 mg, 0.489 mmol) in DCM (2.0 mL) was added in 2M HCl in dioxane (2.0 mL). The reaction mixture was stirred at 20° C. for 2 hrs and was then concentrated under reduced pressure to afford crude 165b (430 mg) which was a white solid and was used in the next step without further purification, LCMS m/z 759.3 (M+H)⁺.

### Step 3: Synthesis of N-(6-{[5-cyclopropyl-1-(oxan-2-yl)-1H-pyrazol-3-yl]amino}-5-methoxy-1,2-benzoxazol-3-yl)-4-[1-(2,2-difluoroethyl)pyrrolidin-2-yl]-2,6-dimethoxy-N-[(4-methoxyphenyl)methyl]benzene-1-sulfonamide (165c)

To a solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(pyrrolidin-2-yl)benzene-1-sulfonamide 165b (165 mg, 0.217 mmol) in DMF (2.17 mL) was added DIPEA (281 mg, 2.17 mmol) and 2,2-difluoroethyl trifluoromethanesulfonate (233 mg, 1.09 mmol). The reaction mixture was stirred at ˜20° C. for 6 hrs and then combined with a smaller batch (50 mg of 165b was used). The resulting mixture was diluted with water (5 ml) and extracted with EtOAc (3×10 mL). The combined organic phase was washed with brine, dried over Na₂SO₄ and concentrated under reduced pressure to give crude 165c (250 mg, >100%) as a yellow oil, which was used in the next step without further purification, LCMS m/z 823.3 (M+H)⁺.

### Step 4: Synthesis of N-{6-[(3-cyclopropyl-1H-pyrazol-5-yl)amino]-5-methoxy-1,2-benzoxazol-3-yl}-4-[1-(2,2-difluoroethyl)pyrrolidin-2-yl]-2,6-dimethoxybenzene-1-sulfonamide (165d)

To a solution of N-(6-{[5-cyclopropyl-1-(oxan-2-yl)-1H-pyrazol-3-yl]amino}-5-methoxy-1,2-benzoxazol-3-yl)-4-[1-(2,2-difluoroethyl)pyrrolidin-2-yl]-2,6-dimethoxy-N-[(4-methoxyphenyl)methyl]benzene-1-sulfonamide 165c (250 mg, 0.304 mmol) in DCM (2 mL) was added TFA (2 mL) at 15° C. The reaction mixture was stirred at ˜15° C. for 16 hrs and was then concentrated under reduced pressure. The crude residue was purified by silica gel column chromatography (eluting with 0-5% MeOH in DCM) to give 165d (120 mg, 80% yield) as a yellow solid, LCMS m/z 619.2 (M+H)⁺. The enantiomers were separated by chiral SFC (50% ethanol with 0.1% ammonium hydroxide in CO₂; 150 mL/min flow rate; Column: Daicel Chiralpak® AD (250×30 mm; 10 μm)).

### N-{6-[(3-cyclopropyl-1H-pyrazol-5-yl)amino]-5-methoxy-1,2-benzoxazol-3-yl}-4-[1-(2,2-difluoroethyl)pyrrolidin-2-yl]-2,6-dimethoxybenzene-1-sulfonamide (Example 165)

The first eluting peak was lyophilized and gave 165 (31.4 mg, 26% yield, 100% ee) as a pink solid. ¹H NMR (400 MHZ, DMSO-d₆) δ 11.99-11.82 (m, 1H), 11.04-10.83 (m, 1H), 8.25-7.98 (m, 2H), 7.52-7.30 (m, 1H), 6.79-6.67 (m, 2H), 6.33-5.84 (m, 1H), 5.83-5.73 (m, 1H), 3.93-3.83 (m, 3H), 3.81-3.72 (m, 6H), 3.60-3.47 (m, 1H), 2.86-2.70 (m, 1H), 2.69-2.55 (m, 2H), 2.46-2.39 (m, 1H), 2.24-2.01 (m, 1H), 1.91-1.72 (m, 3H), 1.60-1.37 (m, 1H), 0.97-0.86 (m, 2H), 0.71-0.60 (m, 2H); LCMS m/z 619.2 (M+H)⁺; [a]_{D}²⁶=+102° (c 0.001, CH₃OH).

### N-{6-[(3-cyclopropyl-1H-pyrazol-5-yl)amino]-5-methoxy-1,2-benzoxazol-3-yl}-4-[1-(2,2-difluoroethyl)pyrrolidin-2-yl]-2,6-dimethoxybenzene-1-sulfonamide (Example 166)

The second eluting peak was lyophilized and gave 166 (32.9 mg, 27% yield, 100% ee) as a pink solid. ¹H NMR (400 MHZ, DMSO-d₆) δ 12.02-11.76 (m, 1H), 11.15-10.74 (m, 1H), 8.27-7.99 (m, 2H), 7.55-7.21 (m, 1H), 6.86-6.57 (m, 2H), 6.22-5.86 (m, 1H), 5.83-5.72 (m, 1H), 3.91-3.84 (m, 3H), 3.79-3.72 (m, 6H), 3.56-3.46 (m, 1H), 2.91-2.70 (m, 1H), 2.69-2.53 (m, 2H), 2.46-2.37 (m, 1H), 2.20-2.06 (m, 1H), 1.92-1.73 (m, 3H), 1.59-1.39 (m, 1H), 0.97-0.84 (m, 2H), 0.73-0.59 (m, 2H); LCMS m/z 619.2 (M+H)⁺; [a]_{D}²⁶=−100° (c 0.001, CH₃OH).

### Examples 167 and 168: N-{6-[(3-cyclopropyl-1H-pyrazol-5-yl)amino]-5-methoxy-1,2-benzoxazol-3-yl}-4-[1-(2,2-difluoroethyl) piperidin-3-yl]-2,6-dimethoxybenzene-1-sulfonamide

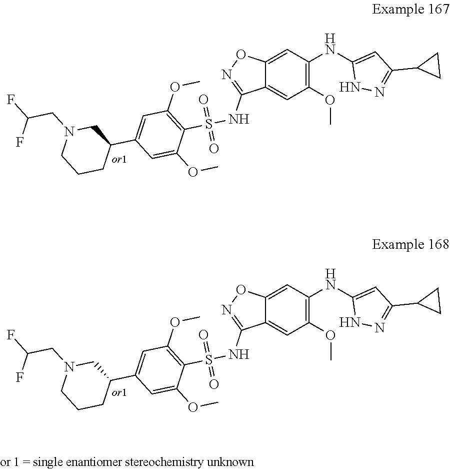 [SMILES: COc1cc2c(NS(=O)(=O)c3c(OC)cc([C@H]4CCCN(CC(F)F)C4)cc3OC)noc2cc1Nc1cc(C2CC2)n[nH]1 ; COc1cc2c(NS(=O)(=O)c3c(OC)cc([C@@H]4CCCN(CC(F)F)C4)cc3OC)noc2cc1Nc1cc(C2CC2)n[nH]1]

Examples 167 and Example 168 were made in a similar manner as Examples 165 and 166 using 1,1-difluoro-2-iodoethane in place of 2,2-difluoroethyl trifluoromethanesulfonate and 158a in place of 165b for step 3. After step 4, the enantiomers were separated by chiral SFC (35% MeOH with 0.1% NH₄OH in CO₂; 80 mL/min flow rate, Daicel Chiralcel OJ, 250×30 mm, 10 μm).

### N-{6-[(3-cyclopropyl-1H-pyrazol-5-yl)amino]-5-methoxy-1,2-benzoxazol-3-yl}-4-[1-(2,2-difluoroethyl) piperidin-3-yl]-2,6-dimethoxybenzene-1-sulfonamide (Example 167)

The first eluting peak was isolated as a white solid (5 mg, 15% yield, >99% ee). ¹H NMR (400 MHz, DMSO-d₆) 0 1H NMR (400 MHZ, DMSO-d₆) δ 11.91 (br. s, 1H), 11.05-10.80 (m, 1H), 8.09 (br. d, J=18.6 Hz, 2H), 7.38 (s, 1H), 6.64 (s, 2H), 6.32-5.93 (m, 1H), 5.77 (s, 1H), 3.86 (s, 3H), 3.75 (s, 6H), 2.91-2.82 (m, 2H), 2.72 (dt, J=4.4, 15.7 Hz, 3H), 2.32-2.16 (m, 2H), 1.89-1.80 (m, 1H), 1.79-1.70 (m, 1H), 1.70-1.61 (m, 1H), 1.61-1.37 (m, 2H), 1.00-0.84 (m, 2H), 0.72-0.59 (m, 2H); LCMS m/z 633.3 (M+H)⁺; [a]_{D}⁵⁰=−8.67° (c 0.1, CH₃OH).

### N-{6-[(3-cyclopropyl-1H-pyrazol-5-yl)amino]-5-methoxy-1,2-benzoxazol-3-yl}-4-[1-(2,2-difluoroethyl) piperidin-3-yl]-2,6-dimethoxybenzene-1-sulfonamide (Example 168)

The second eluting peak was isolated as a white solid (6 mg, 17% yield, 96% ee). ¹H NMR (400 MHz, DMSO-d₆) δ 12.07-11.80 (m, 1H), 10.95 (s, 1H), 8.16-8.04 (m, 2H), 7.40 (s, 1H), 6.64 (s, 2H), 6.47-6.05 (m, 1H), 5.77 (s, 1H), 3.86 (s, 3H), 3.76 (s, 6H), 3.12-2.90 (m, 2H), 2.90-2.74 (m, 2H), 1.88-1.81 (m, 1H), 1.80-1.67 (m, 2H), 1.66-1.40 (m, 2H), 0.96-0.86 (m, 2H), 0.70-0.61 (m, 2H) 3 protons obscured by water and DMSO; LCMS m/z 633.3 (M+H)⁺; [a]_{D}⁵⁰=+6.67° (c 0.1, CH₃OH).

### Example 169 and Example 170: N-{6-[(3-cyclopropyl-1H-pyrazol-5-yl)amino]-5-methoxy-1,2-benzoxazol-3-yl}-2.6-dimethoxy-4-[1-(2,2,2-trifluoroethyl) piperidin-3-yl]benzene-1-sulfonamide

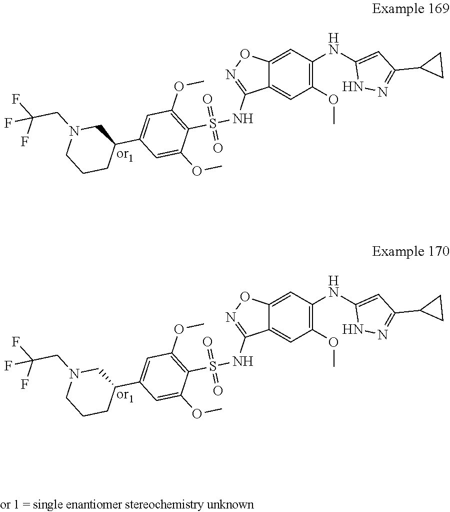 [SMILES: COc1cc2c(NS(=O)(=O)c3c(OC)cc([C@H]4CCCN(CC(F)(F)F)C4)cc3OC)noc2cc1Nc1cc(C2CC2)n[nH]1 ; COc1cc2c(NS(=O)(=O)c3c(OC)cc([C@@H]4CCCN(CC(F)(F)F)C4)cc3OC)noc2cc1Nc1cc(C2CC2)n[nH]1]

Examples 169 and 170were made in a similar manner as Examples 165 and 166 using 2,2,2-trifluoroethyl trifluoromethanesulfonate in place of 2,2-difluoroethyl trifluoromethanesulfonate and 158a in place of 165b for step 3. After step 4, the enantiomers were separated by chiral SFC (30% MeOH with 0.1% NH₄OH in CO₂; 150 mL/min flow rate, Daicel Chiralcel OJ, 250×30 mm, 10 μm).

### N-{6-[(3-cyclopropyl-1H-pyrazol-5-yl)amino]-5-methoxy-1,2-benzoxazol-3-yl}-2,6-dimethoxy-4-[1-(2,2,2-trifluoroethyl) piperidin-3-yl]benzene-1-sulfonamide (Example 169)

The first eluting peak was isolated as a white solid (18 mg, 18%). ¹H NMR (DMSO-d₆, 400 MHZ) δ 11.9-11.8 (m, 1H), 11.0-10.9 (m, 1H), 8.2-8.0 (m, 2H), 7.4-7.3 (m, 1H), 6.7-6.6 (m, 2H), 5.8-5.7 (m, 1H), 3.86 (s, 3H), 3.75 (s, 6H), 3.2-3.1 (m, 2H), 2.89 (br. d, J=10.9 Hz, 2H), 2.8-2.7 (m, 1H), 2.45 (br. s, 1H), 2.4-2.3 (m, 1H), 1.9-1.8 (m, 1H), 1.8-1.7 (m, 1H), 1.7-1.6 (m, 1H), 1.6-1.4 (m, 2H), 0.9-0.9 (m, 2H), 0.7-0.6 (m, 2H); ¹⁹F NMR (DMSO-d₆, 376 MHZ) δ −67.97 (s, 3F) LCMS m/z 651.3 (M+H)⁺; [a]_{D}⁵⁰=−21.71° (c 0.1, CH₃OH).

### N-{6-[(3-cyclopropyl-1H-pyrazol-5-yl)amino]-5-methoxy-1,2-benzoxazol-3-yl}-2,6-dimethoxy-4-[1-(2,2,2-trifluoroethyl)piperidin-3-yl]benzene-1-sulfonamide (Example 170)

The second eluting peak was isolated as a white solid (19 mg, 20%). ¹H NMR (DMSO-d₆, 400 MHz) δ 12.0-11.8 (m, 1H), 10.9-10.9 (m, 1H), 8.2-8.0 (m, 2H), 7.35 (s, 1H), 6.7-6.6 (m, 2H), 5.8-5.7 (m, 1H), 3.86 (s, 3H), 3.75 (s, 6H), 3.2-3.1 (m, 2H), 2.89 (br. d, J=10.9 Hz, 2H), 2.8-2.7 (m, 1H), 2.46 (br. s, 1H), 2.4-2.4 (m, 1H), 1.9-1.8 (m, 1H), 1.8-1.7 (m, 1H), 1.7-1.6 (m, 1H), 1.6-1.4 (m, 2H), 0.9-0.9 (m, 2H), 0.7-0.6 (m, 2H); ¹⁹F NMR (DMSO-d₆, 376 MHZ) δ −68.1-67.9 (m, 3F) LCMS m/z 651.3 (M+H)⁺; [a]_{D}⁵⁰=+5.549° (c 0.1, CH₃OH).

### Examples 171 and 172: N-{6-[(3-cyclopropyl-1H-pyrazol-5-yl)amino]-5-methoxy-1,2-benzoxazol-3-yl}-2,6-dimethoxy-4-(1-methylpiperidin-2-yl)benzene-1-sulfonamide

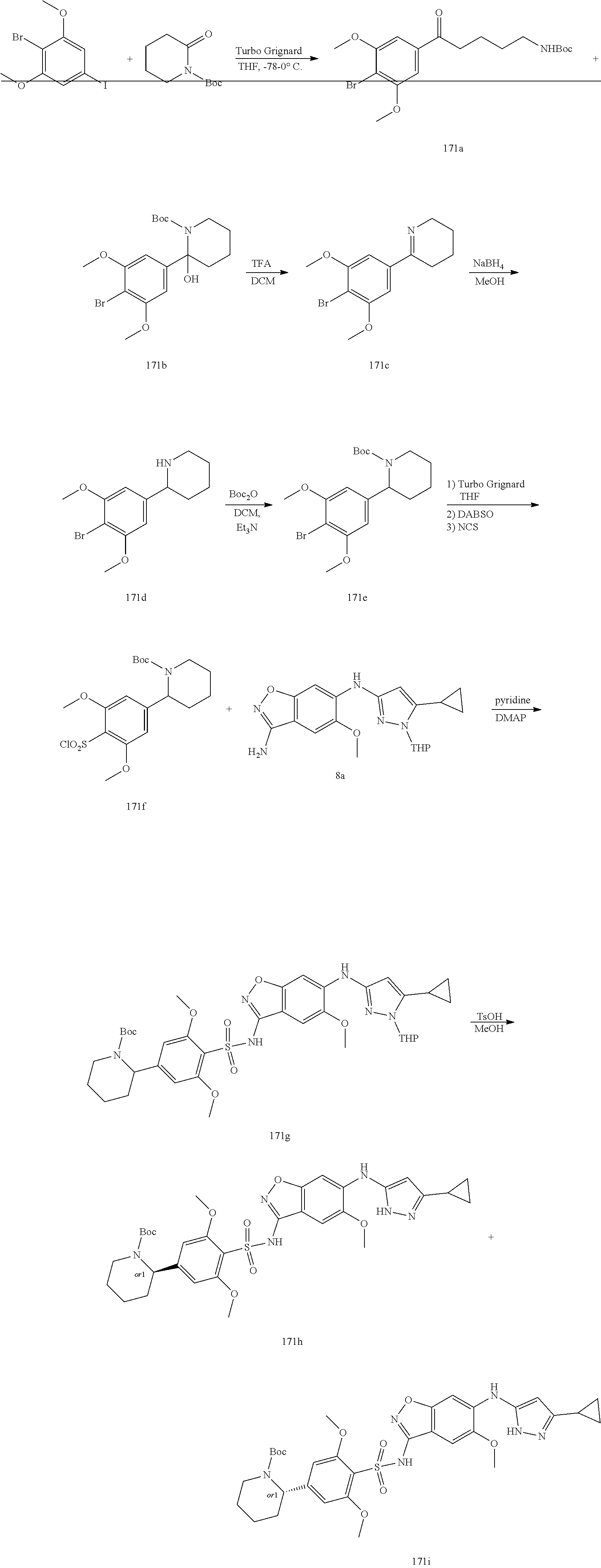

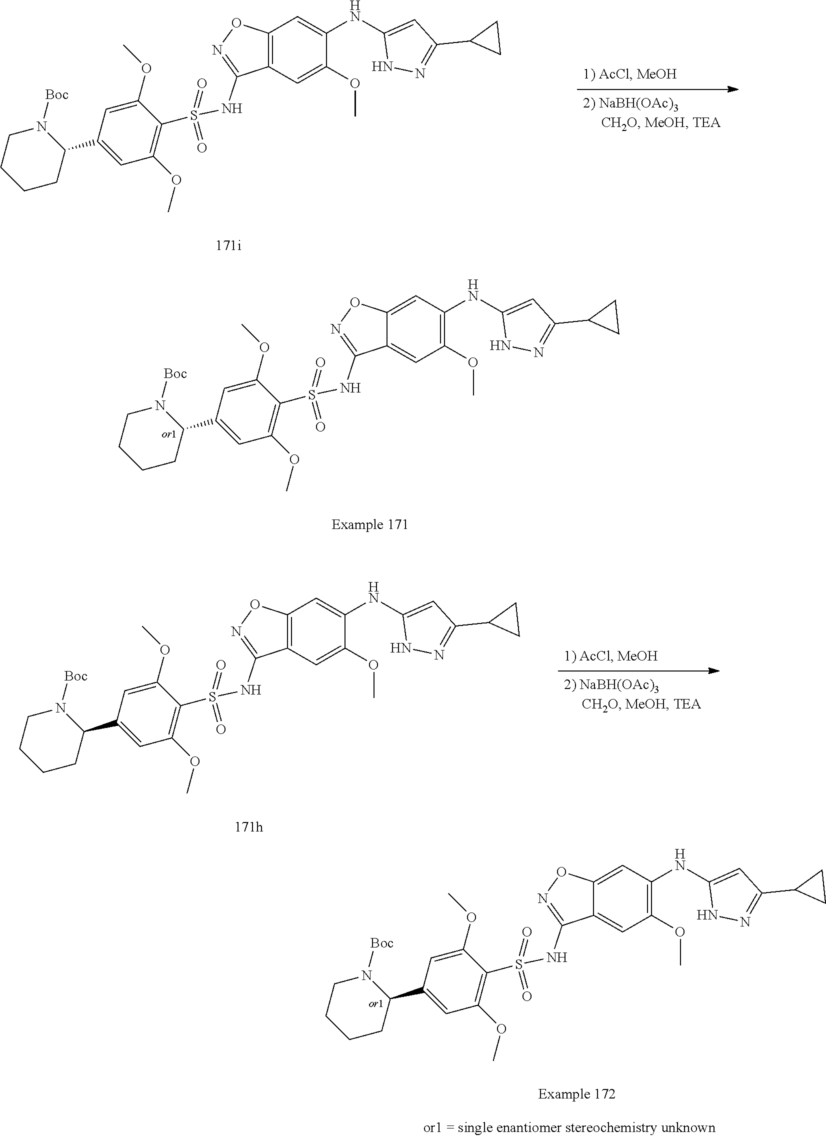 [SMILES: COc1cc2c(NS(=O)(=O)c3c(OC)cc([C@@H]4CCCCN4C(=O)OC(C)(C)C)cc3OC)noc2cc1Nc1cc(C2CC2)n[nH]1 ; COc1cc2c(NS(=O)(=O)c3c(OC)cc([C@@H]4CCCCN4C(=O)OC(C)(C)C)cc3OC)noc2cc1Nc1cc(C2CC2)n[nH]1 ; COc1cc2c(NS(=O)(=O)c3c(OC)cc([C@H]4CCCCN4C(=O)OC(C)(C)C)cc3OC)noc2cc1Nc1cc(C2CC2)n[nH]1 ; COc1cc2c(NS(=O)(=O)c3c(OC)cc([C@H]4CCCCN4C(=O)OC(C)(C)C)cc3OC)noc2cc1Nc1cc(C2CC2)n[nH]1]

### Step 1: Synthesis of tert-butyl [5-(4-bromo-3,5-dimethoxyphenyl)-5-oxopentyl]carbamate (171a) and tert-butyl 2-(4-bromo-3,5-dimethoxyphenyl)-2-hydroxypiperidine-1-carboxylate (171b)

To 2-bromo-5-iodo-1,3-dimethoxybenzene (2.01 g, 5.86 mmol) in THF (29.3 mmol) in an ice bath was added isopropylmagnesium chloride lithium chloride complex (936 mg, 6.45 mmol, 4.96 mL of a 1.3 M solution in THF). After stirring 1 hr, the ice bath was replaced with a dry ice/acetone bath and tert-butyl 2-oxopiperidine-1-carboxylate was added. After 20 mins the dry ice cooling bath was removed, and the mixture brought to rt where it was stirred for 2 hrs. The mixture was cooled in an ice bath and quenched with sat. ammonium chloride solution and partitioned with EtOAc. The organic layer was concentrated under vacuum to afford a mixture of 171a and 171b (2.44 g, 99%) as a golden oil which was used in the next step without further purification. LCMS m/z 316.0, 318.0 (M+H−Boc)⁺.

### Step 2: Synthesis of 6-(4-bromo-3,5-dimethoxyphenyl)-2,3,4,5-tetrahydropyridine (171c)

To a stirred solution of tert-butyl 2-(4-bromo-3,5-dimethoxyphenyl)-2-hydroxypiperidine-1-carboxylate 171b and tert-butyl (5-(4-bromo-3,5-dimethoxyphenyl)-5-oxopentyl) carbamate 171a (1.44 g, 3.46 mmol) in DCM (34 mL) was added TFA (4.4 g, 39 mmol, 3.0 mL). After 5 hrs, the mixture was concentrated under vacuum to afford a brown oil. This was dissolved in DCM/EtOAc/heptanes and concentrated again to remove residual TFA to afford the 171c (1.03 g, 99%) as a brown oil which was used without further purification in the next step.

### Step 3: Synthesis of 2-(4-bromo-3,5-dimethoxyphenyl) piperidine (171d)

To a solution of 6-(4-bromo-3,5-dimethoxyphenyl)-2,3,4,5-tetrahydropyridine 171c (1.03 g, 3.45 mmol) in MeOH (20 mL) was added NaBH₄ (270 mg, 7.14 mmol). The mixture was stirred for 20 mins, then concentrated under vacuum to give 171d (1.04 g, 99%) and used without further manipulation. LCMS m/z 300.0, 302.0 (M+H)⁺.

### Step 4: Synthesis of tert-butyl 2-(4-bromo-3,5-dimethoxyphenyl) piperidine-1-carboxylate (171e)

To a suspension of crude 2-(4-bromo-3,5-dimethoxyphenyl) piperidine 171d (1.04 g, 3.46 mmol) in DCM (25 mL) was added triethylamine (3.50 g, 34.6 mmol) followed by di-tert-butyl dicarbonate (1.51 g, 6.92 mmol). The mixture was stirred for 2 hrs, at which point celite was added and the solvent was removed under vacuum. The material was dry loaded onto an ISCO silica chromatography column and eluted with a 0-30% gradient of EtOAc/heptanes. Evaporation of pooled product fractions afforded 171e (1.19 g, 86%) as a clear colorless oil that crystallized into a white solid on standing. ¹H NMR (400 MHZ, CDCl₃) δ 6.45 (d, J=0.9 Hz, 2H), 5.35 (br. d, J=3.5 Hz, 1H), 4.08 (br. d, J=12.8 Hz, 1H), 3.88 (s, 6H), 2.79 (ddd, J=3.6, 12.1, 13.4 Hz, 1H), 2.26 (br. dd, J=2.3, 14.0 Hz, 1H), 1.92 (ddt, J=3.7, 5.5, 13.5 Hz, 1H), 1.70-1.55 (m, 2H), 1.53-1.36 (m, 11H); LCMS m/z 300.0, 302.0 (M+H−Boc)⁺.

### Step 5: Synthesis of tert-butyl 2-[4-(chlorosulfonyl)-3,5-dimethoxyphenyl]piperidine-1-carboxylate (171f)

A solution of tert-butyl 2-(4-bromo-3,5-dimethoxyphenyl) piperidine-1-carboxylate 171e (1.08 g, 2.70 mmol) in THF (13 mL) at rt was degassed via argon sparge for 10 mins. To the mixture was then added isopropylmagnesium chloride lithium chloride complex (980 mg, 6.74 mmol, 5.19 mL of a 1.3 M solution in THF). The mixture was heated to 60° C. under argon. After 4.5 hrs, the mixture was cooled in a ACN/dry ice bath and DABSO (1.75 g, 7.28 mmol) was added. The reaction was allowed to slowly warm to rt and stirred overnight. To the resultant mixture was then added N-chlorosuccinimide (1.44 g, 10.8 mmol) and the reaction was stirred at rt for 4 hrs. The mixture was diluted with EtOAc, washed sequentially with water and brine, and concentrated. The resultant oil was purified via ISCO chromatography, eluting with 0-80% EtOAc in heptanes, to afford 171f (677 mg, 60%) as a white gum. ¹H NMR (CDCl₃) d 6.50 (s, 2H), 5.35 (br. d, 0.4 Hz, 1H), 4.14-4.09 (m, 1H), 5.89 (s, 6H), 2.79 (m, 1H), 2.21-1.99 (br. dd, 1H), 1.96 (m, 1H), 1.75-1.5 (m, 3H), 1.48 (s, 9H), 1.42-1.38 (m, 1H).

### Step 6: Synthesis of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}piperidine-1-carboxylate (171 g)

To a solution of tert-butyl 2-(4-(chlorosulfonyl)-3,5-dimethoxyphenyl) piperidine-1-carboxylate 171f (178 mg, 424 μmol) and N⁶-(5-cyclopropyl-1-(tetrahydro-2H-pyran-2-yl)-1H-pyrazol-3-yl)-5-methoxybenzo[d]isoxazole-3,6-diamine 8a (157 mg, 425 μmol) in pyridine (2.0 mL) was added DMAP (5.1 mg, 42 μmol), and the mixture was stirred at rt overnight. The mixture was diluted with MeOH and purified by preparative HPLC (ISCO ACCQ Prep HP-125. Luna Omega Polar C18 50×250 mm, 5 mm, flow rate 35 ml/mins. 45-95% ACN: H₂O in 25 mins). The product fractions were combined and most of the ACN removed under vacuum, and the resultant suspension was diluted with ACN until homogeneous. The solution was frozen in a dry ice bath and lyophilized overnight to give 171 g (177 mg, 56%) as a tan powder. ¹H NMR (400 MHZ, CDCl₃) δ 7.99 (br. s, 1H), 7.83 (d, J=1.5 Hz, 1H), 7.45 (s, 1H), 6.99 (s, 1H), 6.44 (s, 2H), 5.66 (s, 1H), 5.48 (dd, J=2.6, 9.9 Hz, 1H), 5.28 (br. s, 1H), 4.12-4.01 (m, 2H), 3.96 (s, 3H), 3.88 (s, 6H), 3.71-3.63 (m, 1H), 2.71 (dt, J=3.2, 12.9 Hz, 1H), 2.59-2.48 (m, 1H), 2.21-2.12 (m, 2H), 2.01-1.83 (m, 3H), 1.81-1.67 (m, 2H), 1.67-1.45 (m, 4H), 1.43 (s, 9H), 1.39-1.30 (m, 1H), 1.02-0.96 (m, 2H), 0.84-0.77 (m, 1H), 0.69-0.62 (m, 1H); LCMS m/z 753.3 (M+H)⁺.

### Step 7: Synthesis of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}piperidine-1-carboxylate (171h) and tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}piperidine-1-carboxylate (171i)

tert-Butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}piperidine-1-carboxylate 171 g (174 mg, 231 μmol) was dissolved in MeOH (12 mL), and p-toluenesulfonic acid monohydrate (10 mg, 53 μmol), was added in four portions over four days. Triethylamine (46.8 mg, 462 μmol) was added and the mixture was concentrated under vacuum. Purification via preparative HPLC (Luna OMega 5 mm polar C18, 250×30 mm, ACN/water w/0.1 eq. formic acid, gradient 25-65%) afforded pure product fractions which were then combined and lyophilized. The enantiomers were separated by chiral SFC (Regis (R,R) Whelk-O1 30 mm×250 mm×10 mm Mobile phase A: CO₂ Mobile phase B: MeOH+10 mm NH₃ 22% B isocratic, 120 bar, 150 mL/mins) to afford compound 171h as the first eluting peak (47.8 mg, 98.0%, >99.0% ee) as a beige powder. ¹H NMR (400 MHZ, DMSO-d₆) δ 11.87 (br. s, 1H), 8.08 (br. s, 1H), 7.95 (br. s, 1H), 7.36 (s, 1H), 6.45 (s, 2H), 5.77 (s, 1H), 5.13 (br. s, 1H), 3.95-3.88 (m, 1H), 3.86 (s, 3H), 3.72 (s, 6H), 2.77 (dt, J=3.1, 12.9 Hz, 1H), 2.25 (br. d, J=12.4 Hz, 1H), 1.85 (tt, J=5.1, 8.4 Hz, 1H), 1.80-1.69 (m, 1H), 1.60-1.47 (m, 2H), 1.45-1.27 (m, 10H), 1.25-1.17 (m, 1H), 0.96-0.88 (m, 2H), 0.70-0.62 (m, 2H); LCMS m/z 669.2 (M+H)⁺; [a]_{D}22=−51.4° (c 0.1, MeOH) and compound 171i as the second eluting peak (38.0 mg, 98.0%, ˜99.0% ee) as a beige powder. ¹H NMR (400 MHZ, DMSO-d₆) δ 11.88 (br s, 1H), 10.94 (br s, 1H), 8.09 (br s, 1H), 7.98 (s, 1H), 7.38 (s, 1H), 6.45 (s, 2H), 5.77 (s, 1H), 5.13 (br. d, J=0.9 Hz, 1H), 3.91 (br. d, J=13.5 Hz, 1H), 3.86 (s, 3H), 3.73 (s, 6H), 2.78 (dt, J=3.1, 12.9 Hz, 1H), 2.24 (br. d, J=13.1 Hz, 1H), 1.85 (tt, J=5.0, 8.4 Hz, 1H), 1.81-1.70 (m, 1H), 1.60-1.48 (m, 2H), 1.46-1.28 (m, 10H), 1.25-1.18 (m, 1H), 0.95-0.88 (m, 2H), 0.69-0.63 (m, 2H); LCMS m/z 669.2 (M+H)⁺; [a]_{D}²²=+60.1° (c 0.1, MeOH)

### Step 8: Synthesis of N-{6-[(3-cyclopropyl-1H-pyrazol-5-yl)amino]-5-methoxy-1,2-benzoxazol-3-yl}-2,6-dimethoxy-4-(1-methylpiperidin-2-yl)benzene-1-sulfonamide (Example 171)

A suspension of 171i (37.0 mg, 55.3 μmol) in MeOH (1.5 mL) was cooled in an ice bath, and acetyl chloride (43.4 mg, 553 μmol, 39.3 mL) was added. The mixture was removed from the ice bath, stirred at rt overnight, and then concentrated under vacuum. The resultant pale-yellow solid was taken back up in MeOH and concentrated a second time. The crude material, N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide, was used in the next step without further purification. LCMS m/z 569.2 (M+H)⁺.

To a suspension of N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide (33.5 mg, 55.4 μmol) in MeOH (1.5 mL) was added in aliquots over 1-2 hrs formalin (3×13.5 mg, 166 μmol) and STAB (2×117 mg, 554 μmol), resulting in little reaction. After storing in the cold overnight, the mixture was warmed to rt and treated with TEA (18 mg, 0.18 mmol). The mixture was held at rt for 25 mins, then stored overnight at ˜5° C. to afford complete reaction. The mixture was filtered through celite and purified by preparative HPLC (Waters XSELECT CSH Prep C18 OBD, 150×19 mm, 5 mm, mobile phase A: Water+10 mM Ammonium Acetate, mobile phase B: ACN) to afford Example 171 (23.4 mg, 73%) as a white solid. Isolated as AcOH salt; ¹H NMR (600 MHZ, DMSO-d₆) δ 11.89 (br. s, 1H), 8.06 (br. s, 1H), 8.01 (s, 1H), 7.35 (s, 1H), 6.66 (s, 2H), 5.76 (s, 1H), 4.14 (br. s, 3H), 3.85 (s, 3H), 3.73 (s, 6H), 2.90 (br. d, J=11.4 Hz, 1H), 2.77 (dd, J=2.0, 11.0 Hz, 1H), 2.00 (dt, J=2.3, 11.8 Hz, 1H), 1.87 (s, 3H), 1.87-1.81 (m, 1H), 1.73-1.50 (m, 4H), 1.41-1.21 (m, 2H), 0.94-0.89 (m, 2H), 0.68-0.63 (m, 2H); LCMS m/z 583.0 (M+H)⁺.

Step 9a: Synthesis of N-{6-[(3-cyclopropyl-1H-pyrazol-5-yl)amino]-5-methoxy-1,2-benzoxazol-3-yl}-2,6-dimethoxy-4-(1-methylpiperidin-2-yl)benzene-1-sulfonamide (Example 172) A suspension of 171h (46.8 mg, 70.0 μmol) in MeOH (1.5 mL) was cooled in an ice bath, and acetyl chloride (110 mg, 1.41 mmol, 100 mL) was added. The solution was removed from the ice bath and stirred at rt overnight. The solvent was removed under vacuum and the solids were dissolved in MeOH and concentrated a second time. The crude material, N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide, was dried overnight under vacuum and was used in the next step without further purification. LCMS m/z 569.2 (M+H)⁺.

To a suspension of N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide (43 mg, 71 μmol) in MeOH (1.5 mL) was added triethylamine (22 mg, 0.21 mmol) followed by formalin (17 mg, 0.21 mmol). After 5 mins, STAB (150 mg, 0.71 mmol) was added. After about 5 mins, the reaction was filtered and concentrated. Purification was accomplished by preparative HPLC (Waters XSELECT CSH Prep C18 OBD, 150×19 mm, 5 mm, water+10 mM ammonium acetate, gradient of 5-95% ACN in 1.7 min, 1 ml/min) to afford Example 172 (30.4 mg, 81%) as a white solid. Isolated as AcOH salt; ¹H NMR (600 MHZ, DMSO-d₆) δ 11.91 (br. s, 1H), 8.05 (br. s, 1H), 8.00 (s, 1H), 7.35 (s, 1H), 6.66 (s, 2H), 5.76 (s, 1H), 4.30-4.00 (m, 3H), 3.85 (s, 3H), 3.73 (s, 6H), 2.90 (br. d, J=11.4 Hz, 1H), 2.77 (dd, J=2.0, 11.0 Hz, 1H), 2.03-1.97 (m, 1H), 1.87 (s, 3H), 1.86-1.82 (m, 1H), 1.72-1.48 (m, 4H), 1.42-1.23 (m, 2H), 0.93-0.88 (m, 2H), 0.67-0.63 (m, 2H); LCMS m/z 583.0 (M+H)⁺.

### Examples 173 and 174: N-{6-[(3-cyclopropyl-1H-pyrazol-5-yl)amino]-5-methoxy-1,2-benzoxazol-3-yl}-4-(1-ethylpiperidin-2-yl)-2,6-dimethoxybenzene-1-sulfonamide

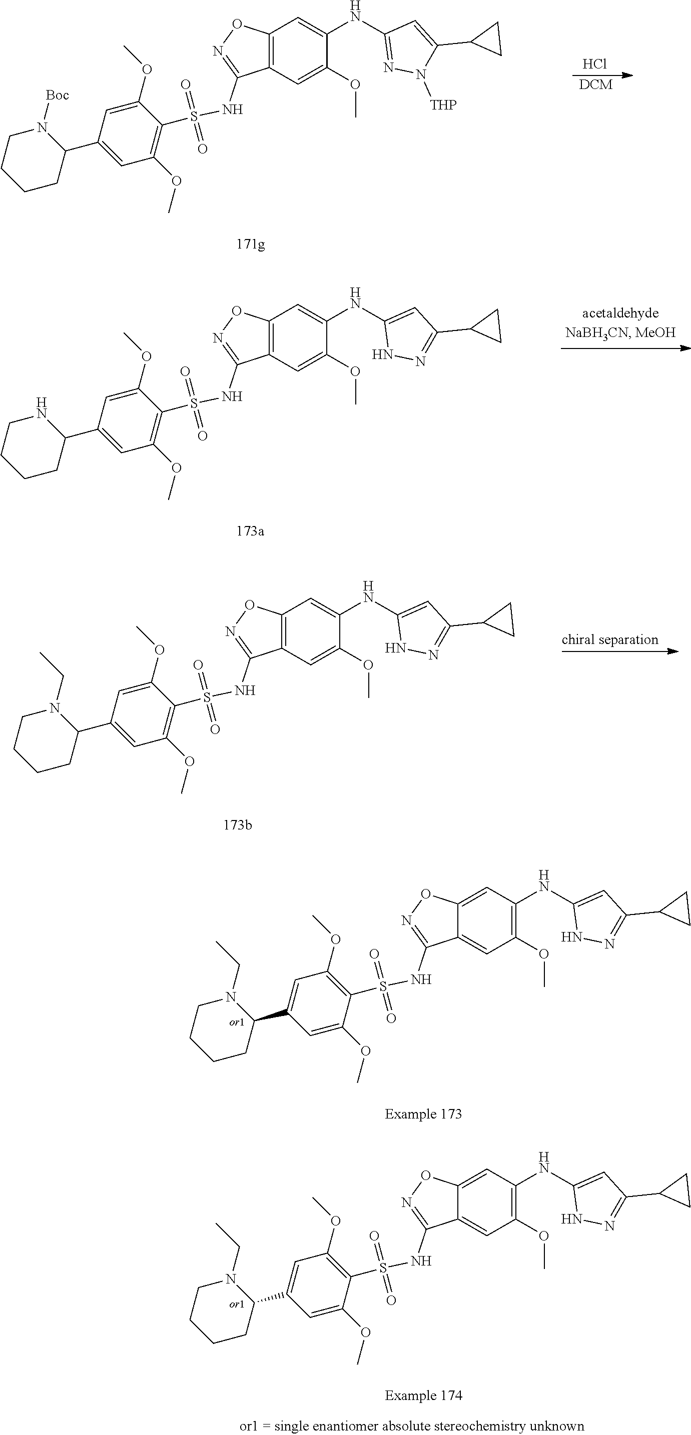

Step 1: Synthesis of N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide (173a) To a solution of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}piperidine-1-carboxylate 171 g (650 mg, 0.86 mmol) in DCM (1 mL) was added HCl in dioxane (3 mL). The reaction mixture was stirred at 20° C. for 16 hrs and then concentrated under reduced pressure to afford crude 173a (600 mg, >100%) which was a white solid and was used in the next step without further purification. LCMS m/z 569.3 (M+H)⁺.

### Step 2: Synthesis of N-{6-[(3-cyclopropyl-1H-pyrazol-5-yl)amino]-5-methoxy-1,2-benzoxazol-3-yl}-4-(1-ethylpiperidin-2-yl)-2,6-dimethoxybenzene-1-sulfonamide (Example 173) and N-{6-[(3-cyclopropyl-1H-pyrazol-5-yl)amino]-5-methoxy-1,2-benzoxazol-3-yl}-4-(1-ethylpiperidin-2-yl)-2,6-dimethoxybenzene-1-sulfonamide (Example 174)

To a suspension of N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide 173a (100 mg, 0.176 mmol) in MeOH (2.2 mL) at 15° C. was added acetaldehyde (77.5 mg, 1.76 mmol). The reaction mixture was stirred at that temperature for 20 min. Sodium cyanoborohydride (55.3 mg, 0.879 mmol) was then added and the mixture was stirred at 15° C. for 1 hr. An additional amount of acetaldehyde (77.5 mg, 1.76 mmol) was added and the reaction was stirred at 15° C. for a further 1 hr. This mixture was combined with a smaller batch (20 mg of 173a was used) and concentrated under reduced pressure. The resulting residue was then purified by silica gel column chromatography (eluting with 0-5% MeOH in DCM) to give racemic 173b (100 mg, combined yield: 79%) as a colorless oil, LCMS m/z 597.3 (M+H)⁺. The enantiomers were separated by chiral SFC (50% isopropanol with 0.1% ammonium hydroxide in CO₂; 150 mL/min flow rate; Column: Daicel Chiralpak® AD (250×30 mm; 10 □m)).

### N-{6-[(3-cyclopropyl-1H-pyrazol-5-yl)amino]-5-methoxy-1,2-benzoxazol-3-yl}-4-(1-ethylpiperidin-2-yl)-2,6-dimethoxybenzene-1-sulfonamide (Example 173)

The first eluting peak was lyophilized and gave 173 (23.04 mg, 23% yield, 100% ee) as a white solid. ¹H NMR (400 MHZ, METHANOL-d₄) δ 7.76-7.50 (m, 1H), 7.38-7.23 (m, 1H), 6.86-6.68 (m, 2H), 5.82-5.69 (m, 1H), 4.02-3.91 (m, 3H), 3.87-3.79 (m, 6H), 3.27-3.17 (m, 2H), 2.57-2.44 (m, 1H), 2.33-2.05 (m, 2H), 1.96-1.87 (m, 1H), 1.85-1.66 (m, 4H), 1.64-1.54 (m, 1H), 1.52-1.41 (m, 1H), 1.02-0.92 (m, 5H), 0.80-0.70 (m, 2H); LCMS m/z 597.3 (M+H)⁺; [a]_{D}²⁶=−62.7° (c 0.001, CH₃OH).

### N-{6-[(3-cyclopropyl-1H-pyrazol-5-yl)amino]-5-methoxy-1,2-benzoxazol-3-yl}-4-(1-ethylpiperidin-2-yl)-2,6-dimethoxybenzene-1-sulfonamide (Example 174)

The second eluting peak was lyophilized and gave 174 (23.24 mg, 23% yield, ˜98.6% ee) as a white solid. ¹H NMR (400 MHZ, METHANOL-d₄) δ 7.75-7.54 (m, 1H), 7.36-7.27 (m, 1H), 6.82-6.68 (m, 2H), 5.85-5.66 (m, 1H), 4.02-3.92 (m, 3H), 3.88-3.81 (m, 6H), 3.28-3.23 (m, 2H), 2.62-2.39 (m, 1H), 2.35-2.04 (m, 2H), 1.97-1.64 (m, 6H), 1.53-1.38 (m, 1H), 1.01-0.95 (m, 5H), 0.78-0.70 (m, 2H); LCMS m/z 597.3 (M+H)⁺; [a]_{D}²⁶=+54.7° (c 0.001, CH₃OH).

### Examples 175 and 176: N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide

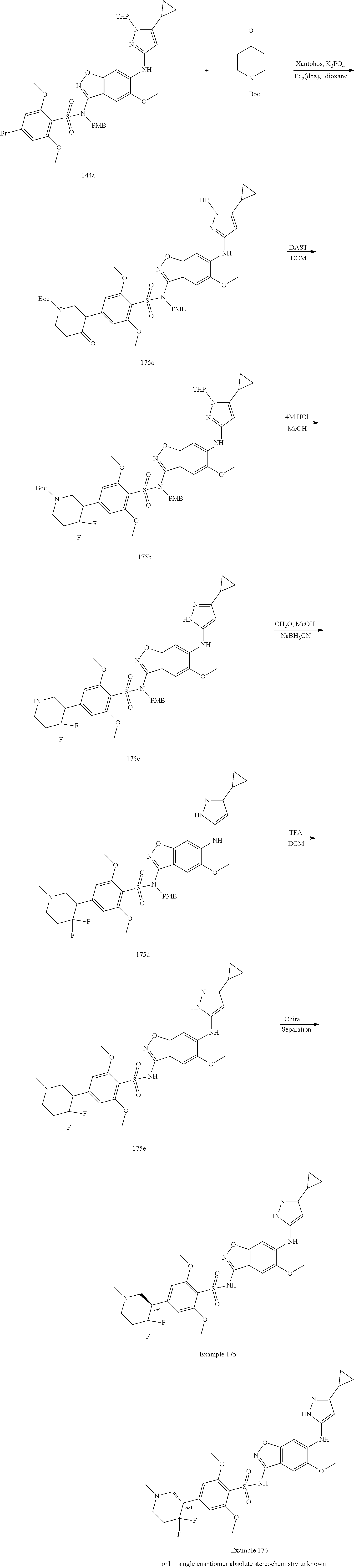

Step 1: tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-4-oxopiperidine-1-carboxylate (175a)

To tert-butyl 4-oxopiperidine-1-carboxylate (600 mg 0.781 mmol) in dioxane (8.0 mL) was added 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide 144a (311 mg, 1.56 mmol), tripotassium phosphate (497 mg, 2.34 mmol), Xantphos (45.2 mg, 2.34 mmol), and Pd₂(dba)₃. The mixture was degassed with nitrogen and stirred at 65° C. for 10 hrs. The mixture was partitioned with water and extracted with EtOAc (3×20 mL). The crude residue was purified by column chromatography (silica gel, 0-60% EtOAc/Pet. ether) to afford 175a (280 mg, 40%). LCMS m/z 887.4 (M+H)⁺.

### Step 2: tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-4,4-difluoropiperidine-1-carboxylate (175b)

To a solution of tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-4-oxopiperidine-1-carboxylate 175a (270 mg, 0.304 mmol) in DCM (3 mL) at ice bath temperature was added DAST (98.1 mg, 0.609 mmol), and the mixture was brought to RT. After stirring for 90 mins, the mixture was quenched with sat. aq NaHCO₃ (10 mL) and extracted with DCM (3×20 mL). The crude residue was purified by column chromatography (12 g silica gel, 0-60% EtOAc/Pet. ether) to afford 175b (170 mg, 61%) as a yellow gum. LCMS m/z 909.4 (M+H)⁺.

### Step 3: N-{6-[(3-cyclopropyl-1H-pyrazol-5-yl)amino]-5-methoxy-1,2-benzoxazol-3-yl}-4-(4,4-difluoropiperidin-3-yl)-2,6-dimethoxy-N-[(4-methoxyphenyl)methyl]benzene-1-sulfonamide (175c)

To tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-4,4-difluoropiperidine-1-carboxylate 175b (170 mg, 0.187 mmol) in MeOH (1 mL) was added 4 M HCl in dioxane (3.0 mL), and the solution was stirred at RT for 16 hrs. The mixture was then concentrated under vacuum to afford crude 175c (130 mg, 95%) which was a yellow solid and was used directly in the next step.

### Step 4: N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxy-N-[(4-methoxyphenyl)methyl]benzene-1-sulfonamide (175d)

To N-{6-[(3-cyclopropyl-1H-pyrazol-5-yl)amino]-5-methoxy-1,2-benzoxazol-3-yl}-4-(4,4-difluoropiperidin-3-yl)-2,6-dimethoxy-N-[(4-methoxyphenyl)methyl]benzene-1-sulfonamide 175c (120 mg, 0.166 mmol) in MeOH (2 mL) at RT was added formalin (50 mg, 1.66 mmol; 37% aq). After stirring for 20 mins, sodium cyanoborohydride (52 mg, 0.828 mmol) was added, and the mixture was stirred for 15 mins. The mixture was filtered through celite, concentrated, and the resulting crude 175d was used directly in the next step. LCMS m/z 739.3 (M+H)⁺.

### Step 5: Synthesis of N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide (175e)

To N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxy-N-[(4-methoxyphenyl)methyl]benzene-1-sulfonamide 175d (150 mg, 0.203 mmol) in DCM (2 mL) at rt was added TFA (4 mL). After 6 hrs, the solvent was removed under vacuum and the residue was purified by preparative HPLC (C18 150×40 mm column, ACN/water w/0.1 eq. formic acid, gradient 17-57% ACN over 9 mins, flow rate 30 mL/mins) to afford 175e (25 mg, 20%). LCMS m/z 619.5 (M+H)⁺.

### Step 6: Synthesis of N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide (Example 175) and N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide (Example 176)

The enantiomers were separated by chiral SFC (Chiralpak AD-3 50×46 mm, 5 mm column eluting with CO₂/EtOH at a flow rate of 4 mL/mins) to afford N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide (Example 175) (3.0 mg, 12%) as the first eluting peak. ¹H NMR (400 MHZ, CDCl₃) δ 7.79 (s, 1H), 7.44 (s, 1H), 6.97 (s, 1H), 6.60 (s, 2H), 5.70 (s, 1H), 3.98 (s, 3H), 3.91 (s, 6H), 2.97-2.81 (m, 2H), 2.63-2.52 (m, 1H), 2.38 (br. s, 3H), 2.21-2.07 (m, 2H), 1.87-1.81 (m, 2H), 1.04-0.96 (m, 2H), 0.77-0.69 (m, 2H); LCMS m/z 619.3 (M+H)⁺.

N-{6-[(3-cyclopropyl-1H-pyrazol-5-yl)amino]-5-methoxy-1,2-benzoxazol-3-yl}-4-(4,4-difluoro-1-methylpiperidin-3-yl)-2,6-dimethoxybenzene-1-sulfonamide (Example) (2.7 mg, 11%) as the second eluting peak. ¹H NMR (400 MHZ, CDCl₃) δ 7.80 (s, 1H), 7.44 (s, 1H), 6.97 (s, 1H), 6.60 (s, 2H), 5.70 (s, 1H), 3.98 (s, 3H), 3.91 (s, 6H), 3.01-2.80 (m, 2H), 2.51-2.34 (m, 4H), 2.24-2.10 (m, 2H), 1.88-1.80 (m, 2H), 1.03-0.97 (m, 2H), 0.79-0.70 (m, 2H); LCMS m/z 619.3 (M+H)⁺.

### Examples 177 and 178: N-{6-[(5-cyclopropyl-1H-pyrazol-3-yl)amino]-5-methoxy-1,2-benzoxazol-3-yl}-4-[4.4-difluoro-1-(²H₃) methylpiperidin-3-yl]-2,6-dimethoxybenzene-1-sulfonamide

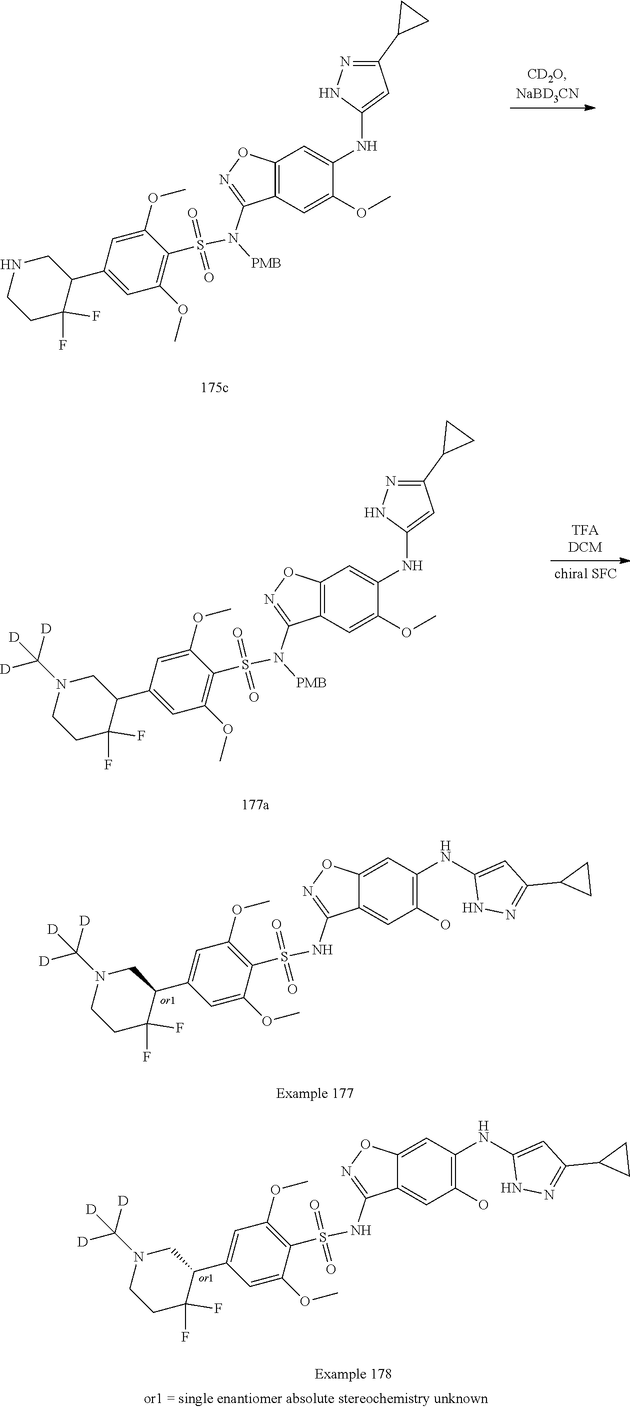

### Step 1: Synthesis of N-{6-[(5-cyclopropyl-1H-pyrazol-3-yl)amino]-5-methoxy-1,2-benzoxazol-3-yl}-4-[4,4-difluoro-1-(²H₃) methylpiperidin-3-yl]-2,6-dimethoxy-N-[(4-methoxyphenyl)methyl]benzene-1-sulfonamide (177a)

To a suspension of N-{6-[(5-cyclopropyl-1H-pyrazol-3-yl)amino]-5-methoxy-1,2-benzoxazol-3-yl}-4-(4,4-difluoropiperidin-3-yl)-2,6-dimethoxy-N-[(4-methoxyphenyl)methyl]benzene-1-sulfonamide (175c) (73 mg, 0.1 mmol) in methanol-d₄ (3 mL) was added 20 wt % D₂O solution of formaldehyde-d₂ solution (81 mg, 0.5 mmol). The reaction mixture was stirred for 10 min at rt. Sodium cyanoborodeuteride (20 mg, 0.3 mmol) was added, and the reaction was stirred at rt for 1 hr. The reaction mixture was concentrated, quenched with water, and extracted with dichloromethane. The organic extracts were combined, concentrated, dissolved in DMSO, and purified using preparatory HPLC (Luna Omega Polar C18 250×30 mm, 5 M, MeCN/water (0.1% formic acid), 25%-65% water at 65 mL/min) to give 177a (65 mg, 87% yield) as a white solid and mixture of enantiomers. ¹H NMR (400 MHZ, CDCl₃-d) δ 7.76 (s, 1H), 7.53 (s, 1H), 7.37 (d, J=8.8 Hz, 2H), 6.91 (s, 1H), 6.75 (d, J=8.8 Hz, 2H), 6.56 (s, 2H), 5.69 (s, 1H), 4.98 (s, 2H), 3.91 (s, 3H), 3.73 (s, 3H), 3.69 (s, 6H), 3.28-3.13 (m, 1H), 2.96-2.85 (m, 2H), 2.60-2.51 (m, 1H), 2.50-2.39 (m, 1H), 2.20-2.11 (m, 2H), 1.88-1.81 (m, 1H), 1.02-0.96 (m, 2H), 0.76-0.70 (m, 2H); LCMS m/z 742.3 (M+H)⁺; rt=1.857.

### Step 2: Synthesis of N-{6-[(5-cyclopropyl-1H-pyrazol-3-yl)amino]-5-methoxy-1,2-benzoxazol-3-yl}-4-[4,4-difluoro-1-(²H₃) methylpiperidin-3-yl]-2,6-dimethoxybenzene-1-sulfonamide (Example 177) and N-{6-[(5-cyclopropyl-1H-pyrazol-3-yl)amino]-5-methoxy-1,2-benzoxazol-3-yl}-4-[4,4-difluoro-1-(²H₃) methylpiperidin-3-yl]-2,6-dimethoxybenzene-1-sulfonamide (Example 178)

To a suspension of N-{6-[(5-cyclopropyl-1H-pyrazol-3-yl)amino]-5-methoxy-1,2-benzoxazol-3-yl}-4-[4,4-difluoro-1-(²H₃) methylpiperidin-3-yl]-2,6-dimethoxy-N-[(4-methoxyphenyl)methyl]benzene-1-sulfonamide (177a) (65 mg, 0.088 mmol) in dichloromethane (1.5 mL) was added trifluoroacetic acid (0.67 mL), and the reaction mixture was stirred at rt for 6 hrs. The reaction mixture was concentrated, taken up in dimethyl sulfoxide, and purified using chiral SFC (ES Industries ChromegaChiral CCO F4 (20 mm×250 mm, 5 μm) CO₂/MeOH (modified by 10 mM ammonia), 18% MeOH isocratic, at a flow rate of 120 mL/min) to give 177 (10.4 mg, 19% yield, >99% ee) as a pale pink solid and the first eluting peak. ¹H NMR (600 MHz, DMSO-d₆) δ 8.14-7.97 (m, 2H), 7.32 (s, 1H), 6.69 (s, 2H), 5.75 (s, 1H), 4.23 (br. s, 1H), 3.84 (s, 3H), 3.73 (s, 6H), 3.38-3.29 (m, 1H), 2.86-2.74 (m, 2H), 2.33-2.23 (m, 1H), 2.11-1.98 (m, 2H), 1.88-1.81 (m, 1H), 0.94-0.87 (m, 2H), 0.68-0.61 (m, 2H); LCMS m/z 622.0 (M+H)⁺; rt=3.415. [a]_{D}22=−11.0° (c 0.1, MeOH).

### N-{6-[(5-cyclopropyl-1H-pyrazol-3-yl)amino]-5-methoxy-1,2-benzoxazol-3-yl}-4-[4,4-difluoro-1-(²H₃) methylpiperidin-3-yl]-2,6-dimethoxybenzene-1-sulfonamide (Example 178)

(9.1 mg, 17% yield, 95% ee) as a white solid and the second eluting peak. ¹H NMR (600 MHZ, DMSO-d₆) δ 8.04 (br. s, 2H), 7.31 (br. d, J=12.6 Hz, 1H), 6.69 (br. d, J=4.8 Hz, 2H), 5.75 (br. s, 1H), 4.22 (br. s, 1H), 3.84 (d, J=3.2 Hz, 3H), 3.73 (br. s, 6H), 3.39-3.29 (m, 1H), 2.86-2.74 (m, 2H), 2.33-2.23 (m, 1H), 2.12-1.99 (m, 2H), 1.88-1.80 (m, 1H), 0.95-0.87 (m, 2H), 0.68-0.60 (m, 2H); LCMS m/z 622.2 (M+H)⁺; rt=3.415. [a]_{D}²²=+1.5° (c 0.1, MeOH).

### Example 179 and 180: N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide

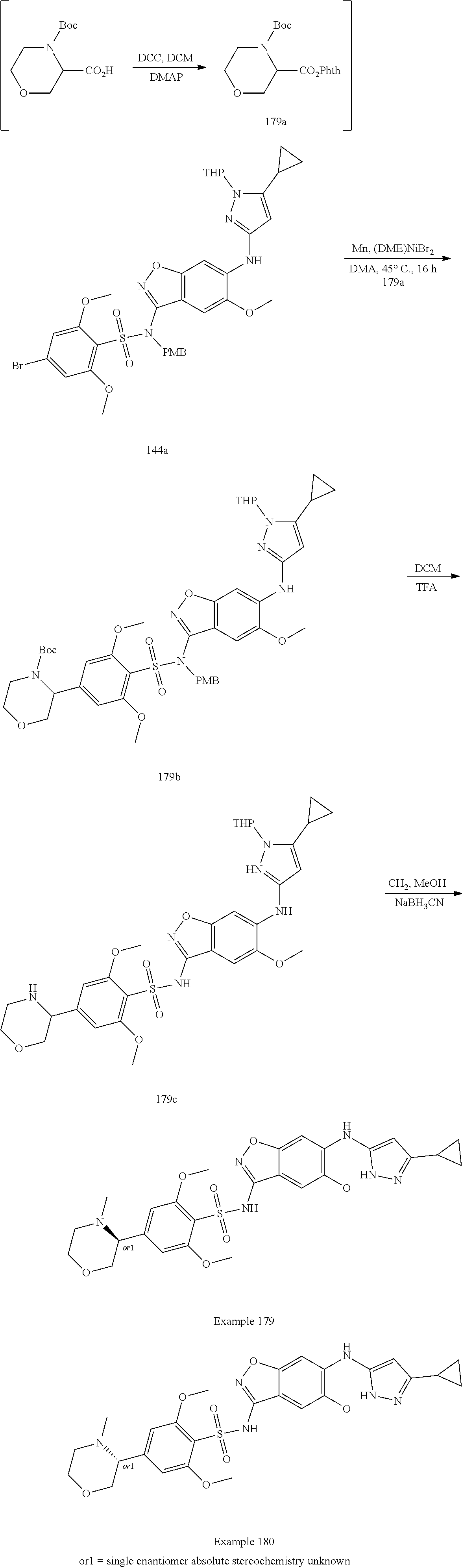

### Step 1: Synthesis of tert-butyl 3-{[(1,3-dioxo-1,3-dihydro-2H-isoindol-2-yl)oxy]carbonyl}morpholine-4-carboxylate (179a)

To a solution of 2-hydroxy-1H-isoindole-1,3 (2H)-dione (320 mg, 1.96 mmol) and DMAP (12.0 mg, 0.091 mmol) in DCM (7 mL) at rt was added 4-(tert-butoxycarbonyl) morpholine-3-carboxylic acid (544 mg, 2.35 mmol). While stirring, N,N′-dicyclohexylcarbodiimide was slowly added in portions. The mixture was stirred for 1 hr and was then filtered through a short pad of silica gel, rinsing with a small amount of DCM. Silica gel chromatography, eluting with 0-20% EtOAc/Pet. ether, afforded 179a (710 mg, 96%) as a white solid. LCMS m/z 276.9 (M+H−Boc)⁺.

### Step 2: Synthesis of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl) morpholine-4-carboxylate (179b)

To 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide 144a (680 mg, 885 mmol), tert-butyl 3-{[(1,3-dioxo-1,3-dihydro-2H-isoindol-2-yl)oxy]carbonyl}morpholine-4-carboxylate (179a) (999 mg, 2.65 mmol), nickel (II) bromide ethylene glycol dimethyl ether complex (273 mg, 885 mmol), N-cyanopyridine-2-carboximidamide (129 mg, 885 mmol), and manganese powder (486 mg, 8.85 mmol) under nitrogen was added DMA (12 mL). The mixture was sparged with nitrogen for 6 mins and was then heated to 45° C. for 18 hrs. The mixture was filtered through celite, rinsing with EtOAc. The filtrate was washed with sat. aq NaHCO₃ (100 mL), and the aqueous wash was extracted with EtOAc (4×80 mL). The combined organic extracts were washed with brine, dried over MgSO₄, and concentrated. The crude residue was purified by column chromatography (40 g silica gel, 0-60% EtOAc/Pet. ether) to afford 179b (400 mg, 52%) as a yellow oil. LCMS m/z 875.4 (M+H)⁺.

### Step 3: Synthesis of N-{6-[(5-cyclopropyl-1H-pyrazol-3-yl)amino]-5-methoxy-1,2-benzoxazol-3-yl}-2,6-dimethoxy-4-(morpholin-3-yl)benzene-1-sulfonamide (179c)

To tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl) morpholine-4-carboxylate 179b (300 mg, 0.343 mmol) in DCM (3 mL) was added TFA (6 mL). The solution was stirred for 10 hrs, and the solvent was then removed under vacuum to afford crude 179c, which was used directly in the next step. LCMS m/z 571.2 (M+H)⁺.

### Step 4: Synthesis of N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide (Example 179) and N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide (Example 180)

To N-{6-[(5-cyclopropyl-1H-pyrazol-3-yl)amino]-5-methoxy-1,2-benzoxazol-3-yl}-2,6-dimethoxy-4-(morpholin-3-yl)benzene-1-sulfonamide 179c (500 mg, 338 mmol) in MeOH (6 mL) was added formalin (711 mg, 8.76 mmol, 37% aq). The mixture was stirred for 15 mins, and sodium cyanoborohydride (275 mg, 4.38 mmol) was added. After stirring for 10 mins, the mixture was filtered and concentrated, and the residue was purified via preparative HPLC (Phenomenex Gemini NX 150×30 mm, 5 mm column H₂O with 0.1% formic acid-ACN, gradient of 15-55% ACN over 9 mins, 60 mL/mins) to afford a racemic mixture of N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide (35 mg, 95%) as a white solid. LCMS m/z 585.3 (M+H)⁺. The enantiomers were separated by chiral SFC (Daicel Chiralcel 250×30 mm, 10 mm column, 50% MeOH with 0.1% ammonium hydroxide in CO₂; 80 mL/mins flow rate)

### N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide (Example 179)

The first eluting peak was isolated as a white solid (10.4 mg, 21%, 98.0% ee). ¹H NMR (400 MHz, DMSO-d₆) δ 11.90 (br. s, 1H), 10.97 (br. s, 1H), 8.16-8.00 (m, 2H), 7.38 (s, 1H), 6.72 (s, 2 H), 5.78 (s, 1H), 3.86 (s, 3H), 3.80 (br. d, J=10.34 Hz, 1H), 3.75 (s, 6H), 3.64-3.55 (m, 2H), 3.20 (br. t, J=10.56 Hz, 1H), 3.04 (dd, J=10.01, 2.97 Hz, 1H), 2.80 (br. d, J=11.88 Hz, 1H), 2.23 (br. d, J=3.08 Hz, 1H), 1.96 (s, 3H), 1.89-1.80 (m, 1H), 0.96-0.82 (m, 2H), 0.69-0.58 (m, 2H); LCMS m/z 585.3 (M+H)⁺; [a]²⁵_{D}=−55.3 (c 0.1, MeOH).

### N-{6-[(5-cyclopropyl-1H-pyrazol-3-yl)amino]-5-methoxy-1,2-benzoxazol-3-yl}-2,6-dimethoxy-4-(4-methylmorpholin-3-yl)benzene-1-sulfonamide (Example 180)

The second eluting peak was isolated as a white solid (7.72 mg, 15%, 98.3% ee). ¹H NMR (400 MHz, DMSO-d₆) δ 11.90 (br. s, 1H), 10.97 (br. s, 1H), 8.16-8.00 (m, 2H), 7.38 (s, 1H) 6.72 (s, 2H), 5.78 (s, 1H), 3.86 (s, 3H), 3.80 (br. d, J=10.34 Hz, 1H), 3.75 (s, 6H), 3.64-3.55 (m, 2H), 3.20 (br. t, J=10.56 Hz, 1H), 3.04 (dd, J=10.01, 2.97 Hz, 1H), 2.80 (br. d, J=11.88 Hz, 1H), 2.23 (br. d, J=3.08 Hz, 1H), 1.96 (s, 3H), 1.89-1.80 (m, 1H), 0.96-0.82 (m, 2H), 0.69-0.58 (m, 2H); LCMS m/z 585.3 (M+H)⁺; [a]²⁵_{D}=+70.6 (c 0.1, MeOH).

### Example 181: N-{6-[(3-cyclopropyl-1H-pyrazol-5-yl)amino]-5-methoxy-1,2-benzoxazol-3-yl}-4-[(4S)-4-fluoro-1-methylpyrrolidin-2-yl]-2,6-dimethoxybenzene-1-sulfonamide

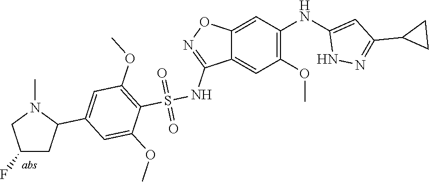 [SMILES: COc1cc2c(NS(=O)(=O)c3c(OC)cc(C4C[C@H](F)CN4C)cc3OC)noc2cc1Nc1cc(C2CC2)n[nH]1]

Example 181 was made in a similar manner as Examples 179 and 180 using 1,3-dioxo-1,3-dihydro-2H-isoindol-2-yl (4S)-1-(tert-butoxycarbonyl)-4-fluoroprolinate in place of tert-butyl 3-{[(1,3-dioxo-1,3-dihydro-2H-isoindol-2-yl)oxy]carbonyl}morpholine-4-carboxylate for step 1.

Example 181: ¹H NMR (400 MHZ, METHANOL-d₄) δ 7.62 (s, 1H), 7.32 (s, 1H), 6.81 (s, 2H), 5.75 (s, 1H), 5.42-5.17 (m, 1H), 3.96 (s, 3H), 3.93-3.76 (m, 6H), 3.01-2.84 (m, 2H), 2.51-2.36 (m, 4H), 2.28-2.07 (m, 2H), 1.94-1.83 (m, 1H), 1.02-0.94 (m, 2H), 0.77-0.69 (m, 2H); LCMS m/z 587.3 (M+H)⁺.

### Example 182: N-{6-[(3-cyclopropyl-1H-pyrazol-5-yl)amino]-5-methoxy-1,2-benzoxazol-3-yl}-4-[(4S)-1,4-dimethylpyrrolidin-2-yl]-2,6-dimethoxybenzene-1-sulfonamide

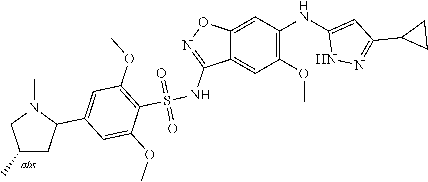 [SMILES: COc1cc2c(NS(=O)(=O)c3c(OC)cc(C4C[C@H](C)CN4C)cc3OC)noc2cc1Nc1cc(C2CC2)n[nH]1]

Example 182 was made in a similar manner as Examples 179 and 180 using 1,3-dioxo-1,3-dihydro-2H-isoindol-2-yl (4S)-1-(tert-butoxycarbonyl)-4-methyl-L-prolinate in place of tert-butyl 3-{[(1,3-dioxo-1,3-dihydro-2H-isoindol-2-yl)oxy]carbonyl}morpholine-4-carboxylate for step 1.

Example 182: 1H NMR (600 MHZ, DMSO-d₆) δ ¹H NMR (600 MHZ, DMSO-d₆) δ=12.00-11.79 (m, 1H), 11.21-10.57 (m, 1H), 8.13-7.97 (m, 2H), 7.36 (s, 1H), 6.70-6.64 (m, 2H), 5.76 (s, 1H), 3.85 (s, 3H), 3.76-3.71 (m, 6H), 3.28-3.11 (m, 2H), 2.37-2.15 (m, 1H), 2.08-2.03 (m, 3H), 1.93-1.82 (m, 2H), 1.78-1.70 (m, 1H), 1.08-0.94 (m, 3H), 0.94-0.88 (m, 2H), 0.68-0.63 (m, 2H), and 1H under the water peak at 3.33; LCMS m/z 583.3 (M+H)⁺.

### Examples 183 and 184: N-{6-[(3-cyclopropyl-1H-pyrazol-5-yl)amino]-5-methoxy-1,2-benzoxazol-3-yl}-4-(6,6-dimethyl-1,4-dioxan-2-yl)-2,6-dimethoxybenzene-1-sulfonamide

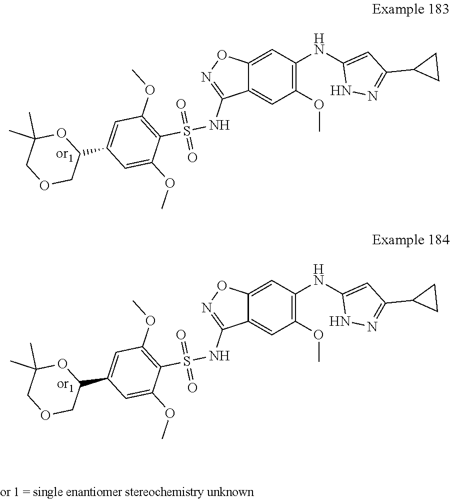

Examples 183 and 184 were made in a similar manner as Examples 179 and 180 using 2-[(6,6-dimethyl-1,4-dioxane-2-carbonyl)oxy]-1H-isoindole-1,3 (2H)-dione in place of tert-butyl 3-{[(1,3-dioxo-1,3-dihydro-2H-isoindol-2-yl)oxy]carbonyl}morpholine-4-carboxylate for step 1 and eliminating the final reductive amination step. The enantiomers were separated by chiral SFC (18% MeOH with 10 mM ammonium in CO₂; 100 mL/min flow rate, 100 bar; Phenomenex Lux Cellulose-1, 250×21.1 mm, 5 μm).

### N-{6-[(3-cyclopropyl-1H-pyrazol-5-yl)amino]-5-methoxy-1,2-benzoxazol-3-yl}-4-(6,6-dimethyl-1,4-dioxan-2-yl)-2,6-dimethoxybenzene-1-sulfonamide (183)

The first eluting peak was isolated as a white solid (5 mg, 8%, 99.0% ee). ¹H NMR (600 MHZ, DMSO-d₆) δ11.90 (br d, J=1.1 Hz, 1H), 11.19-10.54 (m, 1H), 8.12-7.96 (m, 2H), 7.33 (s, 1H), 6.71 (s, 2H), 5.75 (s, 1H), 4.82 (dd, J=2.8, 10.4 Hz, 1H), 1H under broad peak at 4.18, 3.85 (s, 4H), 3.73 (s, 6H), 3.24 (d, J=11.3 Hz, 1H), 3.07 (t, J=10.9 Hz, 1H), 1.88-1.79 (m, 1H), 1.31 (s, 3H), 1.12 (s, 3H), 0.93-0.88 (m, 2H), 0.68-0.62 (m, 2H); LCMS m/z 600.2 (M+H)⁺; [a]_{D}²²=−41.2° (c 0.1, MeOH).

### N-{6-[(3-cyclopropyl-1H-pyrazol-5-yl)amino]-5-methoxy-1,2-benzoxazol-3-yl]-4-(6,6-dimethyl-1,4-dioxan-2-yl)-2,6-dimethoxybenzene-1-sulfonamide (184)

The second eluting peak was isolated as a white solid (7 mg, 11%, 92.0% ee). ¹H NMR (600 MHz, DMSO-d₆) δ ¹H NMR (600 MHZ, DMSO-d₆) δ=11.91 (br s, 1H), 11.06-10.80 (m, 1H), 8.12-7.96 (m, 2H), 7.34 (s, 1H), 6.71 (s, 2H), 5.76 (s, 1H), 4.82 (dd, J=2.8, 10.4 Hz, 1H), 1H under broad peak at 4.18, 3.88-3.83 (m, 4H), 3.77-3.71 (m, 6H), 3.24 (d, J=11.3 Hz, 1H), 3.07 (t, J=10.9 Hz, 1H), 1.84 (tt, J=5.0, 8.4 Hz, 1H), 1.31 (s, 3H), 1.12 (s, 3H), 0.96-0.87 (m, 2H), 0.71-0.58 (m, 2H); LCMS m/z 600.2 (M+H)⁺; [a]_{D}²²=+15.1° (c 0.1, MeOH).

### Example 185 and 186: N-{6-[(3-cyclopropyl-1H-pyrazol-5-yl)amino]-5-methoxy-1,2-benzoxazol-3-yl}-4-(5,5-dimethyl-1,4-dioxan-2-yl)-2,6-dimethoxybenzene-1-sulfonamide

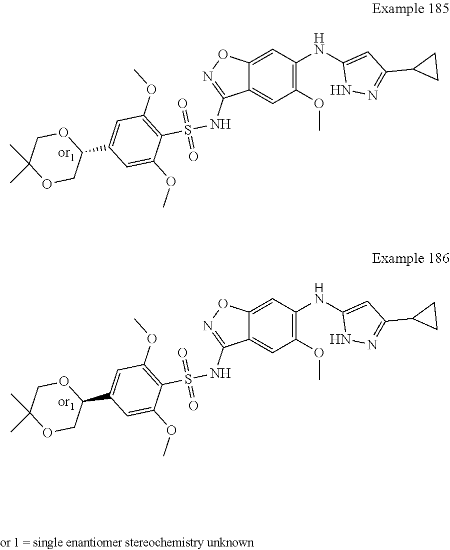 [SMILES: COc1cc2c(NS(=O)(=O)c3c(OC)cc([C@@H]4COC(C)(C)CO4)cc3OC)noc2cc1Nc1cc(C2CC2)n[nH]1 ; COc1cc2c(NS(=O)(=O)c3c(OC)cc([C@H]4COC(C)(C)CO4)cc3OC)noc2cc1Nc1cc(C2CC2)n[nH]1]

Examples 185 and 186 were made in a similar manner as Examples 179 and 180 using 2-[(5,5-dimethyl-1,4-dioxane-2-carbonyl)oxy]-1H-isoindole-1,3 (2H)-dione in place of tert-butyl 3-{[(1,3-dioxo-1,3-dihydro-2H-isoindol-2-yl)oxy]carbonyl}morpholine-4-carboxylate for step 1 and eliminating the final reductive amination step. The enantiomers were separated by chiral SFC (30% MeOH with 10 mM ammonium in CO₂; 4 mL/min flow rate, 120 bar, 40° C.; ChiralCel OJ-H, 150×4.6 mm, 5 μm).

### N-{6-[(3-cyclopropyl-1H-pyrazol-5-yl)amino]-5-methoxy-1,2-benzoxazol-3-yl}-4-(5,5-dimethyl-1,4-dioxan-2-yl)-2,6-dimethoxybenzene-1-sulfonamide (185)

The first eluting peak was isolated as a white solid (15 mg, 23%, 99.0% ee). ¹H NMR (600 MHZ, DMSO-d₆) δ 8.04 (br. s, 1H), 8.01-7.94 (m, 1H), 7.34 (s, 1H), 6.70 (s, 2H), 5.77 (br. s, 1H), 4.45 (dd, J=2.8, 10.3 Hz, 1H), 3.85 (s, 3H), 3.74 (s, 6H), 3.67 (br. d, J=11.4 Hz, 2H), 3.41 (br. s, 2H), 1.89-1.80 (m, 1H), 1.29 (s, 3H), 1.06 (s, 3H), 0.94-0.88 (m, 2H), 0.69-0.62 (m, 2H); LCMS m/z 600.2 (M+H)⁺; [a]_{D}²²=−29.5° (c 0.1, MeOH).

### N-{6-[(3-cyclopropyl-1H-pyrazol-5-yl)amino]-5-methoxy-1,2-benzoxazol-3-yl}-4-(5,5-dimethyl-1,4-dioxan-2-yl)-2,6-dimethoxybenzene-1-sulfonamide (186)

The second eluting peak was isolated as a white solid (15 mg, 23%, 95.0% ee). ¹H NMR (600 MHz, DMSO-d₆) δ 8.03 (br. s, 1H), 7.99 (br. s, 1H), 7.33 (s, 1H), 6.70 (s, 2H), 5.76 (br. s, 1H), 4.45 (br. dd, J=2.5, 10.3 Hz, 1H), 3.85 (s, 3H), 3.74 (s, 6H), 3.66 (br. d, J=11.3 Hz, 2H), 3.41 (br. d, J=11.5 Hz, 2H), 1.91-1.79 (m, 1H), 1.29 (s, 3H), 1.06 (s, 3H), 0.94-0.88 (m, 2H), 0.68-0.62 (m, 2H); LCMS m/z 600.2 (M+H)⁺; [a]_{D}²²=+24.6° (c 0.1, MeOH).

### Example 187: N-{6-[(3-cyclopropyl-1H-pyrazol-5-yl)amino]-5-methoxy-1,2-benzoxazol-3-yl}-2.6-dimethoxy-4-[1-(oxolan-3-yl)azetidin-3-yl]benzene-1-sulfonamide

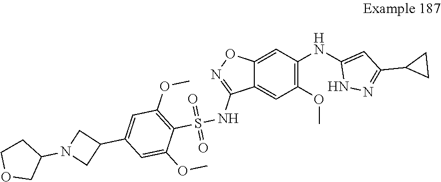 [SMILES: COc1cc2c(NS(=O)(=O)c3c(OC)cc(C4CN(C5CCOC5)C4)cc3OC)noc2cc1Nc1cc(C2CC2)n[nH]1]

Example 187 was made in a similar manner as Examples 179 and 180 using 1-(tert-butyl) 3-(1,3-dioxoisoindolin-2-yl) azetidine-1,3-dicarboxylate in place of 179a for step 1 and dihydrofuran-3 (2H)-one in place of formalin for the last step. The crude product was purified by reverse phase HPLC (10-50% acetonitrile/water with 10 mM NH4OAc; 25 mL/min flow rate in 8 mins, Phenomenex Gemini 5 μm NX-C18 150×21.2 mm, 5 μm) to afford Example 187 (8 mg, 1%) as a racemic mixture (white powder). ¹H NMR (600 MHZ, DMSO-d₆) δ 11.88 (br d, J=2.9 Hz, 1H), 11.22-10.50 (m, 1H), 8.08 (br d, J=1.3 Hz, 1H), 7.98 (s, 1H), 7.37 (s, 1H), 6.64 (s, 2H), 5.77 (s, 1H), 3.86 (s, 3H), 3.76 (s, 6H), 3.72-3.67 (m, 1H), 3.65-3.61 (m, 1H), 3.58-3.50 (m, 3H), 3.42 (br dd, J=2.6, 8.8 Hz, 2H), 3.10-2.99 (m, 3H), 1.85 (s, 1H), 1.75 (s, 1H), 1.64-1.56 (m, 1H), 0.95-0.87 (m, 2H), 0.66 (dd, J=2.2, 5.1 Hz, 2H); LCMS m/z 611.3 (M+H)⁺.

### Examples 188 and 189: N-{6-[(3-cyclopropyl-1H-pyrazol-5-yl)amino]-5-methoxy-1,2-benzoxazol-3-yl}-2,6-dimethoxy-4-(1-methylpyrrolidin-2-yl)benzene-1-sulfonamide

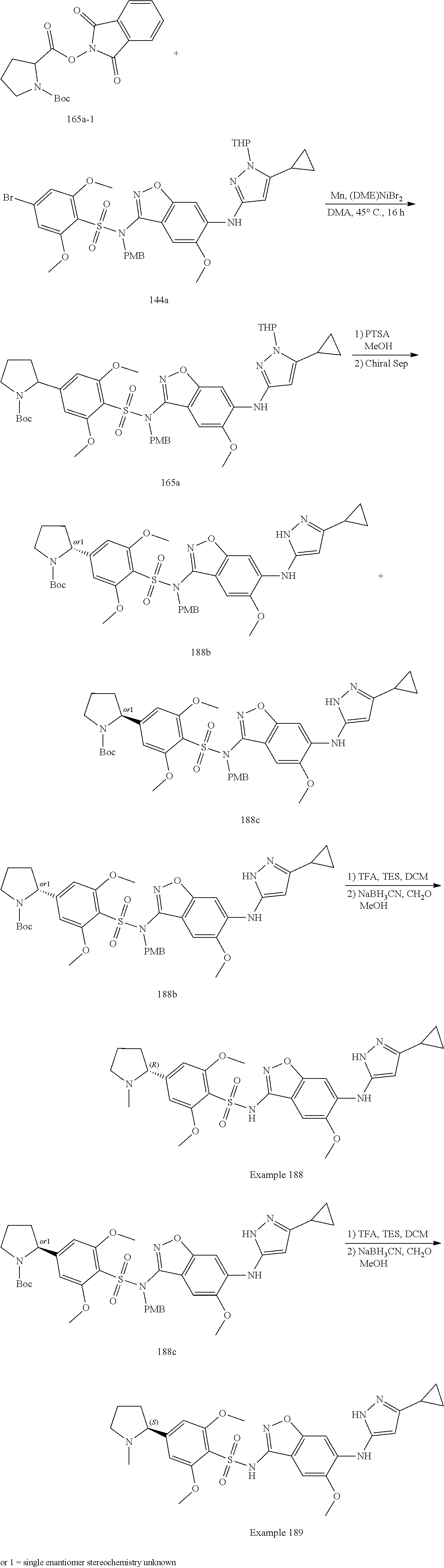

### Synthesis of 1,3-dioxo-1,3-dihydro-2H-isoindol-2-yl 1-(tert-butoxycarbonyl) prolinate 165a-1

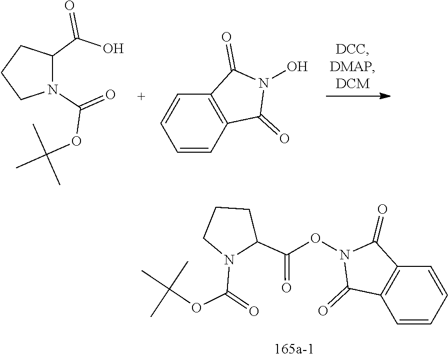 [SMILES: CC(C)(C)OC(=O)N1CCCC1C(=O)O ; O=C1c2ccccc2C(=O)N1O ; CC(C)(C)OC(=O)N1CCCC1C(=O)ON1C(=O)c2ccccc2C1=O]

To a solution of 1-(tert-butoxycarbonyl)pyrrolidine-2-carboxylic acid (1.72 g, 8 mmol) in dry DCM (40 mL) was added N-hydroxyphthalimide (1.31 g, 8 mmol), followed by DCC (1.98 g, 9.6 mmol) and DMAP (97.7 mg, 0.8 mmol) at 20° C. The white reaction mixture was stirred for 16 hrs at 20° C. and was then filtered by a short silica plug and the filter cake was rinsed with DCM (50 mL× 3). The filtrate was concentrated under reduced pressure to give 165a-1 (1.46 g) as a liquid oil, which solidified after 2 hrs. The filter cake was rinsed with DCM (50 mL×3) again and the filtrate was concentrated under reduced pressure to give an additional amount of 165a-1 (1.32 g) as a liquid oil, which also solidified after 2 hrs. The 2 batches were diluted with DCM, combined and concentrated under reduced pressure to afford 165a-1 (2.34 g, 80%) as a white solid ¹H NMR (400 MHZ, CHLOROFORM-d) δ 7.96-7.85 (m, 2H), 7.81 (dd, J=3.1, 5.4 Hz, 2H), 4.62 (dd, J=3.8, 8.8 Hz, 1H), 3.72-3.59 (m, 1H), 3.56-3.45 (m, 1H), 2.53-2.29 (m, 2H), 2.16-1.94 (m, 2H), 1.53 (s, 9H); LCMS m/z 261.1 (M+H−Boc)⁺.

### Step 1: tert-butyl 2-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl]-3,5-dimethoxyphenyl)pyrrolidine-1-carboxylate (165a)

To a vial was added intermediate 144a (344 mg, 448 μmol), 1-tert-butyl 2-(1,3-dioxo-2,3-dihydro-1H-isoindol-2-yl)pyrrolidine-1,2-dicarboxylate (484 mg, 1.34 mmol), Nickel (II) bromide ethylene glycol dimethyl ether complex (138 mg, 448 μmol), (Z)-N′-cyanopicolinimidamide (65.4 mg, 448 μmol) and Manganese (246 mg, 4.48 mmol). The vial was capped, purged with nitrogen then DMA (4.2 mL) was added. The mixture was sparged with nitrogen for 10 mins then placed on a 45° C. heating block with a nitrogen purge. The reaction was stirred for 20 hrs at 45° C. The crude reaction was filtered through a plug of celite and rinsed with EtOAc. The resulting filtrate was washed with sat. sodium bicarbonate solution (20 mL). The aqueous layer was extracted with EtOAc (20 mL). The combined organic layers were washed with brine (20 mL), dried with magnesium sulfate, filtered, and concentrated. The crude residue was purified by column chromatography (ISCO, 24 g silica gel, 0-100% EtOAc/heptanes) to give 165a as an orange solid (115 mg, 30%). ¹H NMR (400 MHZ, DMSO-d₆) δ 8.05 (s, 1H), 7.82 (s, 1H), 7.29 (d, J=8.6 Hz, 2H), 6.84 (d, J=8.6 Hz, 2H), 6.53 (s, 2H), 5.76 (s, 1H), 5.52 (dd, J=2.7, 9.3 Hz, 1H), 5.02 (s, 2H), 4.71 (br dd, J=4.3, 6.8 Hz, 1H), 3.96-3.89 (m, 1H), 3.81 (s, 3H), 3.73 (s, 6H), 3.71 (s, 3H), 3.69-3.60 (m, 1H), 3.59-3.43 (m, 2H), 2.44-2.25 (m, 2H), 2.08 (td, J=3.7, 12.0 Hz, 1H), 1.96-1.88 (m, 2H), 1.88-1.66 (m, 4H), 1.62-1.53 (m, 2H), 1.19 (br t, J=7.1 Hz, 9H), 0.97 (dd, J=2.4, 8.4 Hz, 2H), 0.72-0.63 (m, 1H), 0.63-0.55 (m, 1H); LCMS m/z 859.3 (M+H)⁺.

### Step 2: Synthesis of tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188b) and tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188c)

To a solution of tert-butyl 2-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)pyrrolidine-1-carboxylate 165a (165 mg, 192 μmol) in MeOH (10 mL) was added p-toluenesulfonic acid monohydrate (18 mg, 95 μmol). The reaction was stirred at rt for 2 hrs. Additional p-toluenesulfonic acid monohydrate (11 mg, 58 μmol) was added, and the reaction was stirred for an additional 18 hrs. The reaction was neutralized with TEA (38.9 mg, 53.5 μL, 384 μmol) then concentrated and dried on high vacuum. The enantiomers were separated by chiral SFC (35% MeOH with 10 mm NH₃ in CO₂; 4 mL/mins flow rate; 40° C.; 120 bar; Phenomenex Lux cell-2, 150 mm×4.6 mm, 5 μm).

### tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188b)

The first eluting peak was isolated as a white solid (27 mg, 18% yield). LCMS m/z 775.5 (M+H)⁺; [a]_{D}²²=+42.1° (c 0.1, MeOH).

### tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188c)

The second eluting peak was isolated as a white solid (27 mg, 18% yield). LCMS m/z 775.4 (M+H)⁺; [a]_{D}²²=−42.4° (c 0.1, MeOH).

### Step 3: Synthesis of N-{6-[(3-cyclopropyl-1H-pyrazol-5-yl)amino]-5-methoxy-1,2-benzoxazol-3-yl}-2,6-dimethoxy-4-[(2R)-1-methylpyrrolidin-2-yl]benzene-1-sulfonamide (Example 188)

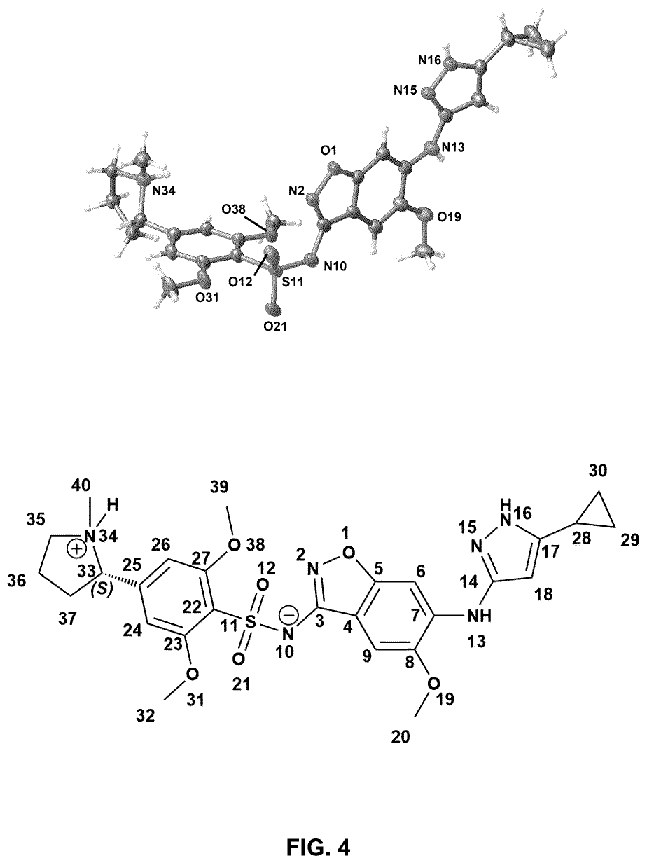

To a solution of tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188b) (26.5 mg, 34.2 μmol) in DCM (171 μL) was added triethylsilane (7.95 mg, 68.4 μmol) followed by TFA (331 mg, 2.91 mmol). After storing overnight, the solvent was removed under vacuum. The resulting material (N-{6-[(3-cyclopropyl-1H-pyrazol-5-yl)amino]-5-methoxy-1,2-benzoxazol-3-yl}-2,6-dimethoxy-4-(pyrrolidin-2-yl)benzene-1-sulfonamide) was taken forward without purification (19 mg, 100%). LCMS m/z (ESI+) for 555.2 (M+H)⁺. To the crude residue (19 mg, 34 μmol) was added MeOH (0.43 mL) followed by sodium cyanoborohydride (6.5 mg, 0.10 mmol) and formaldehyde (10 mg, 0.34 mmol). The mixture was stirred for 10 mins and was then concentrated and purified by preparative HPLC (Phenomenex Gemini NX-C18 4.6×50 mm×5 μm column; Mobile phase A: aqueous 10 mM NH4OAc+2% ACN, Mobile phase B: ACN, 0-80% over 5.0 mins, flow rate 2.25 mL/mins) to afford Example 188 (7.1 mg, 37%) as a white solid. ¹H NMR (600 MHZ, DMSO-d₆) δ 12.14-11.69 (m, 1H), 8.03 (br s, 1H), 7.94 (br s, 1H), 7.32 (s, 1H), 6.66 (s, 2H), 5.76 (s, 1H), 3.84 (s, 3H), 3.71 (s, 6H), 3.15-3.08 (m, 1H), 3.05 (t, J=8.3 Hz, 1H), 2.21 (q, J=9.0 Hz, 1H), 2.16-2.06 (m, 4H), 1.89-1.81 (m, 1H), 1.81-1.75 (m, 1H), 1.71 (ddd, J=2.8, 5.9, 8.7 Hz, 1H), 1.57-1.47 (m, 1H), 0.94-0.87 (m, 2H), 0.69-0.62 (m, 2H); LCMS m/z 569.0 (M+H)⁺; [a]_{D}²²=+47.3° (c 0.06, MeOH). Example 188 was characterized by chiral SFC 40% isopropanol+10 mM NH₃ in CO₂ in CO₂; 4 mL/min flow rate, 120 bar; Chiralpak AD-3; 100×4.6 mm, 3 m) with a retention time of 6.407 min (peak 2). The absolute stereochemistry of Example 188 was determined to be (R) owing to it being the opposite enantiomer of Example 189 that demonstrated an absolute stereochemistry of (S) by single-crystal X-ray crystallography. See FIG. 4.

### Step 3a: Synthesis of N-{6-[(3-cyclopropyl-1H-pyrazol-5-yl)amino]-5-methoxy-1,2-benzoxazol-3-yl}-2,6-dimethoxy-4-[(2S)-1-methylpyrrolidin-2-yl]benzene-1-sulfonamide (Example 189)

To a solution of tert-butyl 2-[4-({6-[(3-cyclopropyl-1H-pyrazol-5-yl)amino]-5-methoxy-1,2-benzoxazol-3-yl}[(4-methoxyphenyl)methyl]sulfamoyl)-3,5-dimethoxyphenyl]pyrrolidine-1-carboxylate (188c) (26.6 mg, 34.3 mmol) in DCM (0.172 mL) was added triethylsilane (8.0 mg, 0.069 mmol) followed by TFA (331 mg, 2.91 mmol), and the solution was stirred overnight. The mixture was concentrated under vacuum and the resulting material (N-{6-[(3-cyclopropyl-1H-pyrazol-5-yl)amino]-5-methoxy-1,2-benzoxazol-3-yl}-2,6-dimethoxy-4-(pyrrolidin-2-yl)benzene-1-sulfonamide) was used directly in the next step (19 mg, 100%). LCMS m/z 555.2 (M+H)⁺. To a solution of the crude residue (19 mg, 34 μmol) in MeOH (0.43 mL) was added sodium cyanoborohydride (6.5 mg, 0.10 mmol) and formaldehyde (10 mg, 0.34 mmol). The mixture was stirred for 10 mins then concentrated and purified by preparative HPLC (Phenomenex Gemini NX-C18 4.6×50 mm×5 mm column; Mobile phase A: aqueous 10 mM NH₄OAc+2% ACN, Mobile phase B: ACN 0-80% over 5.0 mins, flow rate 2.25 mL/mins) to afford Example 189 (8.4 mg, 44%) as a white solid. ¹H NMR (600 MHZ, DMSO-d₆) δ 12.27-11.51 (m, 1H), 7.98 (br s, 1H), 7.86 (br s, 1H), 7.27 (s, 1H), 6.64 (s, 2H), 5.75 (s, 1H), 3.83 (s, 3H), 3.68 (s, 6H), 3.14-3.08 (m, 1H), 3.06-3.01 (m, 1H), 2.21 (q, J=9.0 Hz, 1H), 2.15-2.06 (m, 4H), 1.87-1.81 (m, 1H), 1.81-1.75 (m, 1H), 1.71 (ddd, J=2.9, 5.9, 8.8 Hz, 1H), 1.53 (tdd, J=5.5, 8.9, 12.3 Hz, 1H), 0.94-0.87 (m, 2H), 0.68-0.63 (m, 2H); LCMS m/z 569.2 (M+H)⁺; [a]_{D}²²=−57.1° (c 0.06, MeOH). Example 189 was characterized by chiral SFC 40% isopropanol+10 mM NH₃ in CO₂ in CO₂; 4 mL/min flow rate, 120 bar; Chiralpak AD-3; 100×4.6 mm, 3 m) with a retention time of 5.442 min (peak 1). The absolute stereochemistry of Example 189 was determined to be(S) by single crystal X-ray diffraction studies. Crystals of Example 189 were grown from EtOH/H₂O and data were collected in a nitrogen gas stream at 100 (2) K. See FIG. 4.

### Example 189 (stereospecific procedure): N-{6-[(3-cyclopropyl-1H-pyrazol-5-yl)amino]-5-methoxy-1.2-benzoxazol-3-yl}-2,6-dimethoxy-4-[(2S)-1-methylpyrrolidin-2-yl]benzene-1-sulfonamide

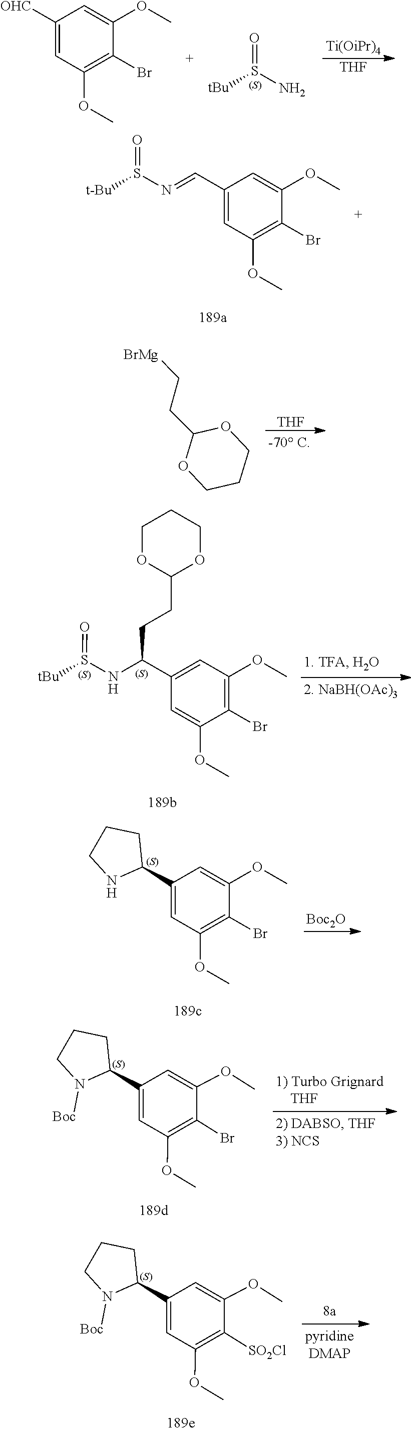

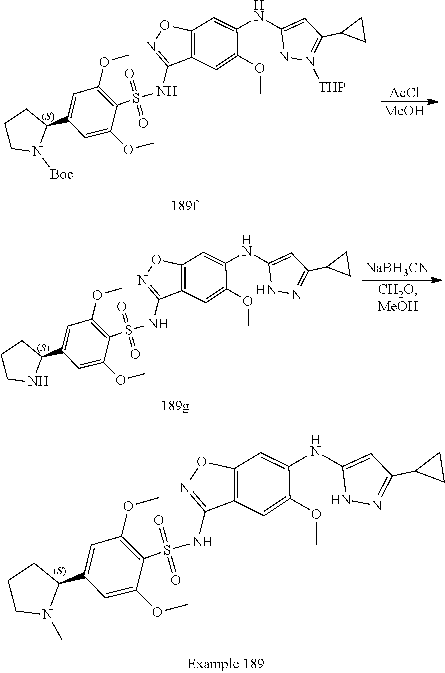

### Step 1: Synthesis of (S S)-N-[(E)-(4-bromo-3,5-dimethoxyphenyl)methylidene]-2-methylpropane-2-sulfinamide (189a)

To a solution of 4-bromo-3,5-dimethoxybenzaldehyde (4000 mg, 16.32 mmol) in THF (163 mL) was added titanium (IV) isopropoxide (9.278 g, 9.72 mL, 32.64 mmol) at rt. The reaction was stirred for 10 mins then(S)-(−)-2-methylpropane-2-sulphinamide (1.978 g, 16.32 mmol) was added and stirring was continued at rt for 48 hrs. Starting material remained as evidenced by TLC. Additional(S)-(−)-2-Methylpropane-2-sulphinamide (0.5 eq, 989 mg) was added and stirring was continued for 6 hrs at rt. The reaction was quenched with water and brine and diluted with THF. The mixture was filtered through celite then the layers were separated, and the organic phase was dried (Na₂SO₄), filtered and concentrated. The crude residue was purified by column chromatography (ISCO, SiO₂, eluting with 0-50% EtOAc in heptane) to afford 189a (4.59 g, 81%) as a white solid. ¹H NMR (400 MHZ, CDCl₃) 0 8.53 (s, 1H), 7.07 (s, 2H), 3.98 (s, 6H), 1.29 (s, 9H); LCMS m/z 350.0 (M+H)⁺.

### Step 2: Synthesis of (S²S)-N-[(1S)-1-(4-bromo-3,5-dimethoxyphenyl)-3-(1,3-dioxan-2-yl)propyl]-2-methylpropane-2-sulfinamide (189b)

A flask was charged with 189a (2000 mg, 5.743 mmol) and dry THF (5 mL) and cooled to −73° C. A solution of (1,3-dioxan-2-ylethyl) magnesium bromide (0.5 M in THF) (4.386 g, 28.71 mL, 14.36 mmol) was then added dropwise over 30 mins maintaining an internal temperature of −68° C. After addition, the reaction was stirred for another 30 mins then additional (1,3-dioxan-2-ylethyl) magnesium bromide (0.5 M in THF) (4.386 g, 28.71 mL, 14.36 mmol) was added dropwise maintaining an internal temperature of −68° C. After 1 hr the reaction reached −50° C., and sat. aq. NH₄Cl (20 mL, 0.38 M) was added. The crude reaction was extracted with EtOAc (× 3). The combined organic phases were washed with brine, then dried over sodium sulfate, filtered and concentrated. The crude residue was purified by column chromatography (ISCO, SiO₂, eluting with 0-100% EtOAc in heptane) to afford 189b (2.06 g, 77%) as a clear oil. ¹H NMR (400 MHz, CDCl₃) δ 6.57 (s, 2H), 4.50 (t, J=4.94 Hz, 1H), 4.34-4.27 (m, 1H), 4.11-4.06 (m, 2H), 3.90 (s, 6H), 3.79-3.68 (m, 2H), 2.15-2.06 (m, 1H), 2.02-1.99 (m, 1H), 1.92-1.81 (m, 1H), 1.68-1.45 (m, 3H), 1.24 (s, 9H).

### Step 3: Synthesis of (2S)-2-(4-bromo-3,5-dimethoxyphenyl)pyrrolidine (189c)

A mixture of TFA (44 g, 30 mL, 88 eq, 0.39 mol) and water (30 g, 30 mL, 1.7 mol) were cooled in an ice bath for 20 mins. This mixture was added to a cold (ice bath) flask containing 189b (2.06 g, 4.44 mmol). The reaction was stirred in an ice bath for 3 hrs, then placed in the fridge overnight. The reaction was then cooled in an ACN/dry ice bath (bath temperature ˜−30° C.) and STAB (2.10 g, 9.91 mmol) was added in 3 portions every 5 mins. The reaction was allowed to warm to rt then concentrated to give crude 189c (1.27 g, 100%) as a cloudy oil.

### Step 4: Synthesis of tert-butyl (2S)-2-(4-bromo-3,5-dimethoxyphenyl)pyrrolidine-1-carboxylate (189d)

A solution of crude 189c (1.27 g, 4.44 mmol) in 2-Methyltetrahydrofuran (90 mL) and sat. aq. NaHCO₃ (100 mL) was cooled in an ice bath. Di-tert-butyl dicarbonate (1.45 g, 1.49 mL, 6.65 mmol) and DMAP (75 mg, 0.14 eq, 0.61 mmol) were added and the biphasic mixture was stirred vigorously for 18 hrs, during which time the ice bath melted. The layers were separated, and the organic layer was washed with brine then concentrated. The crude residue was purified by column chromatography (ISCO, SiO₂, eluting with 0-70% EtOAc in heptane) to afford 189d (1.5 g, 87%) as a clear gum. ¹H NMR (400 MHZ, CDCl₃) δ 6.39 (s, 2H), 4.83-4.67 (m, 1H), 3.87 (s, 6H), 3.69-3.56 (m, 2H), 2.38-2.27 (m, 1H), 2.00-1.75 (m, 3H), 1.52-1.18 (m, 9H); LCMS m/z 286 (M+H−Boc)⁺.

### Step 5: Synthesis of tert-butyl (2S)-2-[4-(chlorosulfonyl)-3,5-dimethoxyphenyl]pyrrolidine-1-carboxylate (189e)

A solution of tert-butyl (2S)-2-(4-bromo-3,5-dimethoxyphenyl)pyrrolidine-1-carboxylate (189d) (1.32 g, 3.42 mmol) in THF (16 mL) was degassed for 10 mins with argon, then isopropylmagnesium chloride lithium chloride complex solution (1.24 g, 6.57 mL, 1.3 M in THF, 8.54 mmol) was added slowly. The reaction was heated to 60° C. for 3.5 hrs then cooled to rt then further cooled to −40° C. DABSO (2.13 g, 8.88 mmol) was ground into a powder then added as a solid under nitrogen to the reaction at −40° C. After 5 min at −40° C., the cold bath was removed and the reaction was allowed to warm to rt over 45 mins. The reaction was again cooled to −5° C. in a brine/ice bath and NCS (1.83 g, 1.13 mL, 13.7 mmol) was added in one portion under nitrogen. After stirred in the cooling bath for 5 mins with high stirring, the reaction mixture was quenched with H₂O and EtOAc. The aqueous phase was extracted with EtOAc (×1) and MTBE (×1) to break up the emulsion. The combined organic extracts were washed with brine, dried over MgSO₄, filtered and concentrated. The crude residue was purified by column chromatography (ISCO, SiO₂, 0-70% EtOAc in heptane) to give 189e (810 mg, 58%) as a white foam. ¹H NMR (400 MHZ, CDCl₃) δ 6.55-6.41 (m, 2H), 4.97-4.68 (m, 1H), 3.98 (s, 6H), 3.72-3.54 (m, 2H), 2.47-2.30 (m, 1H), 1.86 (d, J=1.0 Hz, 3H), 1.51-1.17 (m, 9H); LCMS m/z 386 (M-Cl+O)⁻.

### Step 6: Synthesis of tert-butyl (2S)-2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}pyrrolidine-1-carboxylate (189f)

To a flask was added N6-(5-cyclopropyl-1-(tetrahydro-2H-pyran-2-yl)-1H-pyrazol-3-yl)-5-methoxybenzo[d]isoxazole-3,6-diamine (8a) (567 mg, 1.53 mmol), DMAP (18.8 mg, 153 μmol) and 4 Å Molecular Sieves (1.7 g, 1.53 mmol). The mixture was evacuated and backfilled with nitrogen once. Anhydrous pyridine (3.07 mL) was added, and the reaction was stirred for 5 mins to allow a homogeneous solution then tert-butyl(S)-2-(4-(chlorosulfonyl)-3,5-dimethoxyphenyl)pyrrolidine-1-carboxylate (189e) (810 mg, 2.00 mmol) was added. The reaction was stirred under a nitrogen atmosphere for 18 hrs. The crude reaction was diluted with EtOAc and 1N HCl (25 mL). The layers were separated, and the organic phase was washed brine then dried (MgSO₄), filtered and concentrated. The crude residue was purified by column chromatography (ISCO, SiO₂, 0-100% EtOAc/Heptane) to give 189f (930 mg, 82%) as a white solid. ¹H NMR (400 MHZ, CDCl₃) δ 7.98 (s, 1H), 7.77 (d, J=4.0 Hz, 1H), 7.48 (s, 1H), 7.11 (br d, J=4.0 Hz, 1H), 6.39 (s, 2H), 5.66 (s, 1H), 5.49 (dd, J=2.6, 9.8 Hz, 1H), 4.91-4.58 (m, 1H), 4.12-4.04 (m, 1H), 3.96 (s, 3H), 3.88 (s, 6H), 3.72-3.52 (m, 3H), 2.60-2.46 (m, 1H), 2.38-2.24 (m, 1H), 2.19-2.10 (m, 1H), 1.97 (br dd, J=2.6, 12.9 Hz, 1H), 1.92-1.82 (m, 3H), 1.81-1.68 (m, 3H), 1.68-1.53 (m, 2H), 1.51-1.39 (m, 2H), 1.17-1.05 (m, 6H), 1.04-0.95 (m, 2H), 0.89-0.77 (m, 1H), 0.72-0.61 (m, 1H); LCMS m/z 739.3 (M+H)⁺.

### Step 7: Synthesis of N-{6-[(3-cyclopropyl-1H-pyrazol-5-yl)amino]-5-methoxy-1,2-benzoxazol-3-yl}-2,6-dimethoxy-4-[(2S)-pyrrolidin-2-yl]benzene-1-sulfonamide (189 g)

To a cooled (0° C.) solution of 189f (930 mg, 1.26 mmol) in MeOH (18.0 mL) was added acetyl chloride (1.28 g, 1.16 mL, 16.4 mmol) The reaction was to warm to rt and stirred for 18 hrs. The reaction was concentrated and dried under high vacuum. Crude 189 g was used in the next step without further purification. LCMS m/z 555.2 (M+H)⁺.

### Step 8: Synthesis of N-{6-[(3-cyclopropyl-1H-pyrazol-5-yl)amino]-5-methoxy-1,2-benzoxazol-3-yl}-2,6-dimethoxy-4-[(2S)-1-methylpyrrolidin-2-yl]benzene-1-sulfonamide (Example 189)

To a vial containing 189 g (653 mg, 1.18 mmol) was added sodium cyanoborohydride (222 mg, 3.52 mmol), MeOH (15 mL) and formaldehyde (353 mg, 0.324 mL, 11.8 mmol). The reaction was stirred at rt for 1 hr then concentrated. The crude solid was purified by SFC (15-40% methanol in CO₂; 100 mL/min flow rate, 120 bar; Cosmosil 3-Hydroxyphenyl; 150×20 mm, 5 μm) to give 189 (361 mg, 54%) as a white solid. Example 189 was characterized by chiral SFC (40% isopropanol+10 mM NH₃ in CO₂; 4 mL/min flow rate, 120 bar; Chiralpak AD-3; 100×4.6 mm, 3 μm) and compared to a racemic sample. Example 189 was determined to be peak 1 with a retention time of 6.223 min and >99.0% ee. The absolute stereochemistry was confirmed by X-ray crystallography. ¹H NMR (600 MHz, DMSO-d₆) δ 8.07-8.01 (m, 1H), 7.39 (s, 1H), 6.91-6.78 (m, 2H), 5.76 (s, 1H), 3.86 (s, 3H), 3.78 (s, 6H), 3.20-3.12 (m, 2H), 2.44-2.17 (m, 5H), 1.99-1.81 (m, 4H), 0.96-0.87 (m, 2H), 0.70-0.63 (m, 2H) (large water peak observed); LCMS m/z 569 (M+H)⁺.

### Example 190 and 191: N-{6-[(3-cyclopropyl-1H-pyrazol-5-yl)amino]-5-methoxy-1,2-benzoxazol-3-yl}-4-(1-ethylpyrrolidin-2-yl)-2,6-dimethoxybenzene-1-sulfonamide

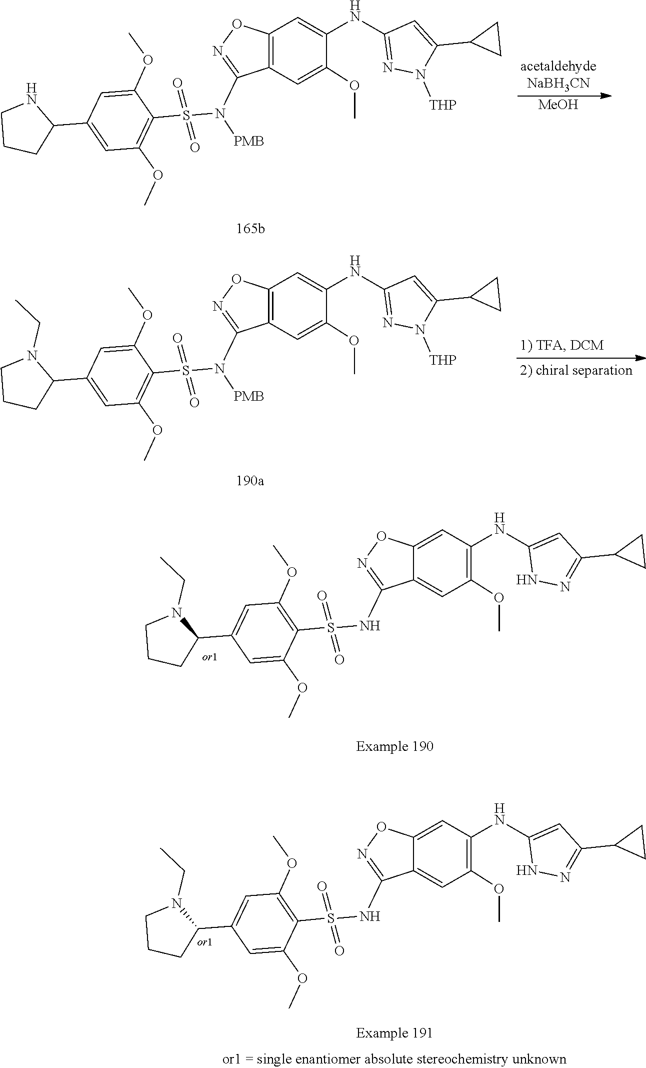

### Step 1: Synthesis of N-(6-{[5-cyclopropyl-1-(oxan-2-yl)-1H-pyrazol-3-yl]amino}-5-methoxy-1,2-benzoxazol-3-yl)-4-(1-ethylpyrrolidin-2-yl)-2,6-dimethoxy-N-[(4-methoxyphenyl)methyl]benzene-1-sulfonamide (190a)

190a was prepared in a similar manner as Example 173b using N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(pyrrolidin-2-yl)benzene-1-sulfonamide 165b (150 mg, 0.198 mmol) in place of N-{6-[(3-cyclopropyl-1H-pyrazol-5-yl)amino]-5-methoxy-1,2-benzoxazol-3-yl}-2,6-dimethoxy-4-(piperidin-2-yl)benzene-1-sulfonamide 173a. The resulting crude mixture was combined with a smaller batch (30 mg of 165b was used) and was then diluted with water (5 mL) and extracted with EtOAc (3×10 mL). The combined organic phase was washed with brine, dried over Na₂SO₄ and concentrated under reduced pressure to give crude 190a (200 mg, crude yield: 82%) as a yellow oil, which was used in the next step without further purification, LCMS m/z 787.3 (M+H)⁺.

### Step 3: Synthesis of N-{6-[(5-cyclopropyl-1H-pyrazol-3-yl)amino]-5-methoxy-1,2-benzoxazol-3-yl}-4-(1-ethylpyrrolidin-2-yl)-2,6-dimethoxybenzene-1-sulfonamide (Example 190 and Example 191)

To a solution of N-(6-{[5-cyclopropyl-1-(oxan-2-yl)-1H-pyrazol-3-yl]amino}-5-methoxy-1,2-benzoxazol-3-yl)-4-(1-ethylpyrrolidin-2-yl)-2,6-dimethoxy-N-[(4-methoxyphenyl)methyl]benzene-1-sulfonamide 190a (200 mg, 0.15 mmol) in DCM (2 mL) was added TFA (2 mL) at 15° C. The reaction mixture was stirred at ˜15° C. for 16 hrs and was then concentrated under reduced pressure. The crude residue was purified by silica gel column chromatography (eluting with 0-100% EtOAc in Pet. ether) to give a racemic mixture of Example 190 and 191 (120 mg, 81% yield) as a yellow oil, LCMS m/z 583.2 (M+H)⁺. The enantiomers were separated by chiral SFC (50% ethanol with 0.1% ammonium hydroxide in CO₂; 80 mL/min flow rate; Column: Daicel Chiralpak® IG (250×30 mm; 10 □m)).

### N-{6-[(5-cyclopropyl-1H-pyrazol-3-yl)amino]-5-methoxy-1,2-benzoxazol-3-yl}-4-(1-ethylpyrrolidin-2-yl)-2,6-dimethoxybenzene-1-sulfonamide (Example 190)

The first eluting peak was lyophilized and gave Example 190 (15.97 mg, 13% yield, 100% ee) as a white solid. ¹H NMR (400 MHZ, DMSO-d₆) δ 11.98-11.82 (m, 1H), 11.07-10.77 (m, 1H), 8.21-7.98 (m, 2H), 7.46-7.29 (m, 1H), 6.75-6.63 (m, 2H), 5.86-5.67 (m, 1H), 3.94-3.82 (m, 3H), 3.77-3.71 (m, 6H), 3.28-3.19 (m, 2H), 2.48-2.45 (m, 1H), 2.23-1.98 (m, 3H), 1.92-1.64 (m, 3H), 1.58-1.39 (m, 1H), 1.00-0.88 (m, 5H), 0.68-0.57 (m, 2H); LCMS m/z 583.2 (M+H)⁺; [a],29=−40° (C. 0.001, CH₃OH).

### N-{6-[(5-cyclopropyl-1H-pyrazol-3-yl)amino]-5-methoxy-1,2-benzoxazol-3-yl}-4-(1-ethylpyrrolidin-2-yl)-2,6-dimethoxybenzene-1-sulfonamide (Example 191)

The second eluting peak was lyophilized and gave Example 191 (12.34 mg, 10% yield, 100% ee) as a white solid. ¹H NMR (400 MHZ, DMSO-d₆) δ 12.07-11.78 (m, 1H), 11.09-10.69 (m, 1H), 8.25-7.90 (m, 2H), 7.52-7.25 (m, 1H), 6.91-6.50 (m, 2H), 5.91-5.65 (m, 1H), 3.92-3.80 (m, 3H), 3.78-3.66 (m, 6H), 3.29-3.27 (m, 2H), 2.45-2.43 (m, 1H), 2.25-1.93 (m, 3H), 1.92-1.66 (m, 3H), 1.57-1.35 (m, 1H), 1.01-0.83 (m, 5H), 0.70-0.57 (m, 2H), LCMS m/z 583.2 (M+H)⁺; [a]_{D}²⁹=+38° (C. 0.001, CH₃OH).

### Examples 192 and 193: N-{6-[(3-cyclopropyl-1H-pyrazol-5-yl)amino]-5-methoxy-1,2-benzoxazol-3-yl}-4-[(4R)-4-fluoro-1-methylpiperidin-2-yl]-2,6-dimethoxybenzene-1-sulfonamide

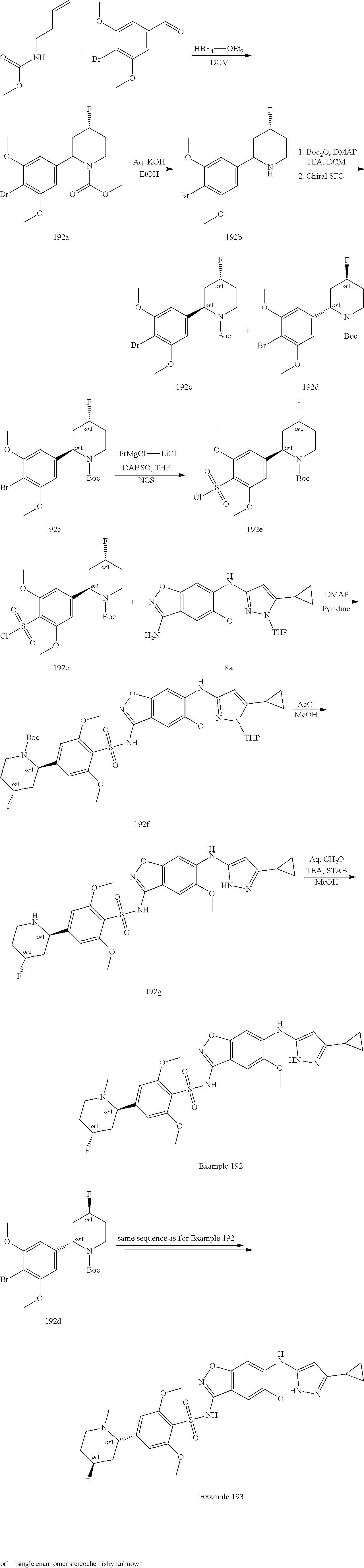

### Step 1: Synthesis of methyl trans 2-(4-bromo-3,5-dimethoxyphenyl)-4-fluoropiperidine-1-carboxylate (192a)

To a solution of 4-bromo-3,5-dimethoxybenzaldehyde (6.83 g, 27.9 mmol) and methyl but-3-en-1-ylcarbamate (3.60 g, 27.9 mmol) in DCM (100 mL) in an ice bath was added tetrafluoroboric acid-diethyl ether complex (4.54 mL, 33.4 mmol). The solution was stirred for 5 mins and then removed from the ice bath. After 4 hrs, the reaction was quenched with the addition of silica gel. The DCM was removed under vacuum, and the resulting powder was dry loaded (Silica gel, EtOAc/Heptane 0-80%) to afford 192a as a clear gum (4.62 g, 44%). LCMS m/z 376/378 (M+H)⁺; ¹H NMR (400 MHZ, CHLOROFORM-d) δ 6.44 (d, J=0.8 Hz, 2H), 5.61 (br s, 1H), 4.70 (ttd, J=4.6, 10.5, 49.0 Hz, 1H), 4.32-4.23 (m, 1H), 3.89 (s, 6H), 3.78 (s, 3H), 2.89 (dddd, J=1.4, 3.0, 13.0, 14.3 Hz, 1H), 2.70 (tdt, J=2.3, 4.3, 10.8 Hz, 1H), 2.10 (br s, 1H), 2.10-2.00 (m, 1H), 1.73 (dtt, J=5.4, 11.0, 12.9 Hz, 1H).

### Step 2: Synthesis of trans 2-(4-bromo-3,5-dimethoxyphenyl)-4-fluoropiperidine (192b)

To a solution of (192a) (4.6 g, 12 mmol) in EtOH (100 mL) was added 10M Aq. potassium hydroxide (20 mL, 0.20 mol). The reaction was heated to 80° C. for 40 hrs, 95° C. for 8 hrs and then 90° C. for an additional 18 hrs. The reaction was allowed to cool to rt, and then Sat. Aq. NaHCO₃ was added and stirred vigorously for 5 mins. The mixture was extracted with EtOAc. The organic layer was concentrated and dried under vacuum. The resulting orange gum was dissolved in ACN and concentrated again. The resulting brown solid (192b) was used without further purification.

LCMS m/z 318/320 (M+H)⁺; ¹H NMR (400 MHZ, CHLOROFORM-d) δ 6.64 (s, 2H), 5.11-4.92 (m, 1H), 4.01 (dd, J=2.5, 11.8 Hz, 1H), 3.91 (s, 6H), 3.24-3.15 (m, 1H), 3.05 (ddd, J=1.9, 5.3, 11.7 Hz, 1H), 2.13 (qdd, J=2.8, 11.2, 14.0 Hz, 1H), 2.06-1.95 (m, 1H), 1.92-1.71 (m, 2H). One proton under H₂O peak.

### Step 3: Synthesis of tert-butyl trans-2-(4-bromo-3,5-dimethoxyphenyl)-4-fluoropiperidine-1-carboxylate (192c) and tert-butyl trans-2-(4-bromo-3,5-dimethoxyphenyl)-4-fluoropiperidine-1-carboxylate (192d)

To a solution of crude (192b) (3.08 g, 9.68 mmol, theoretical mass from step 2), TEA (2.70 mL, 19.4 mmol), and DMAP (237 mg, 1.94 mmol) in an ice bath was added di-tert-butyl dicarbonate (3.47 g, 15.9 mmol). The reaction was removed from the ice bath and stirred at rt. overnight. Celite was added to the reaction mixture, and the solvent was removed under vacuum. This was used for dry loading (silica gel, 0-60% EtOAc/Heptanes) to afford a yellow solid (2.48 g, 61%). LCMS m/z 318/320 (M+H)⁺. The enantiomers were separated by chiral SFC (Regis (R,R) Whelk-O1 10/100 21.1 mm×250 mm Mobile phase A: CO2 Mobile phase B: MeOH 9% B isocratic, 120 bar, 100 mL/min)

### tert-butyl trans-2-(4-bromo-3,5-dimethoxyphenyl)-4-fluoropiperidine-1-carboxylate (192c)

The first eluting peak was isolated as a white solid (979 mg, 39% yield). LCMS m/z 318/320 (M-Boc+H)⁺; ¹H NMR (400 MHZ, DMSO-d₆) δ 6.52 (d, J=0.6 Hz, 2H), 5.37 (br s, 1H), 4.79-4.55 (m, 1H), 4.12-4.01 (m, 1H), 3.82 (s, 6H), 2.89 (dt, J=2.6, 13.4 Hz, 1H), 2.80-2.68 (m, 1H), 2.07-1.88 (m, 2H), 1.67-1.51 (m, 1H), 1.40 (s, 9H); ¹⁹F NMR (377 MHZ, DMSO-d₆) δ −172.51 (s, 1F); [a]_{D}²²=−64.4 (c 0.1, MeOH).

### tert-butyl trans-2-(4-bromo-3,5-dimethoxyphenyl)-4-fluoropiperidine-1-carboxylate (192d)

The second eluting peak was isolated as a white solid (994 mg, 40% yield). LCMS m/z 318/320 (M-Boc+H)⁺; ¹H NMR (400 MHZ, DMSO-d₆) δ 6.52 (d, J=0.5 Hz, 2H), 5.37 (br s, 1H), 4.83-4.52 (m, 1H), 4.13-4.02 (m, 1H), 3.82 (s, 6H), 2.89 (dt, J=2.6, 13.4 Hz, 1H), 2.79-2.68 (m, 1H), 2.06-1.88 (m, 2H), 1.67-1.51 (m, 1H), 1.40 (s, 9H); ¹⁹F NMR (377 MHZ, DMSO-d₆) δ −172.51 (s, 1F); [a]_{D}²²=+66.8 (c 0.1, MeOH).

### Step 4: Synthesis of tert-butyl trans-2-[4-(chlorosulfonyl)-3,5-dimethoxyphenyl]-4-fluoropiperidine-1-carboxylate (192e)

A solution of 192c (0.421 g, 1.01 mmol) in THF (5.0 mL) at rt. was degassed by bubbling argon through for 10 minutes. To this was added Turbo Grignard Solution (1.9 mL, 1.3 molar in THF, 2.5 mmol). The mixture was heated to 60° C. under argon. The reaction was transferred to an ACN/dry ice bath. To this was added DABSO (605 mg, 2.52 mmol). The reaction was allowed to continue overnight, during which time it warmed to rt. To this was then added NCS (538 mg, 4.03 mmol) and the reaction mixture was stirred at rt for 4 hrs. The resulting suspension was diluted with EtOAc and washed with water and brine. The organic layer was concentrated, and the crude residue was purified by flash column chromatography (Silica gel, 0-80% EtOAc/Heptanes) to give (192e) (139 mg, 32%, 65% pure) as a clear gum. 19F NMR (377 MHZ, CHLOROFORM-d) δ −174.65 (s, 1F).

### Step 5: Synthesis of tert-butyl trans-2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}-4-fluoropiperidine-1-carboxylate (192f)

To a solution of (192e) (139 mg, 317 μmol) and (8a) (118 mg, 319 μmol) in pyridine was added DMAP (5 mg, 0.1 eq, 0.04 mmol). The solution was stirred at rt for 18 hrs. The crude reaction mixture was diluted with methanol and purified by reverse phase HPLC (ISCO ACCQ Prep HP-125. Luna Omega Polar C18 21×250 mm, 5 μm, flow rate 35 ml/min. 45-95% ACN: H₂O in 25 min). The pure fractions were combined, concentrated and lyophilized to afford (192f) as a white powder (63 mg, 26%). LCMS m/z 771.2 (M+H)⁺; ¹⁹F NMR (377 MHZ, CHLOROFORM-d) 0-174.52 (br d, J=4.2 Hz, 1F).

### Step 6: Synthesis of N-{6-[(5-cyclopropyl-1H-pyrazol-3-yl)amino]-5-methoxy-1,2-benzoxazol-3-yl}-4-trans (4-fluoropiperidin-2-yl)-2,6-dimethoxybenzene-1-sulfonamide (192 g)

A suspension of (192f) (61 mg, 79 μmol) in methanol (2.0 mL) was cooled in an ice bath, and acetyl chloride (110 mg, 100 μL, 18 eq, 1.41 mmol) was added. The reaction was removed from the ice bath and stirred at rt for 1 day. The solvent was removed under vacuum and dried overnight under high vacuum. This material was used as crude 192 g for the next step. LCMS m/z 587.1 (M+H)⁺.

### Step 7: Synthesis of N-{6-[(5-cyclopropyl-1H-pyrazol-3-yl)amino]-5-methoxy-1,2-benzoxazol-3-yl}-4-trans (4-fluoro-1-methylpiperidin-2-yl)-2,6-dimethoxybenzene-1-sulfonamide (Example 192)

To a solution of (192 g) (39 mg, 1 eq, 66 μmol) in methanol (1.5 mL) was added TEA (22 mg, 30 μL, 3.2 eq, 0.22 mmol). The reaction became cloudy. To this was added Aq. Formaldehyde solution (15 μL, 37% Wt, 0.20 mmol). After 5 mins, STAB (70 mg, 0.33 mmol) was added. After 5 mins, the reaction mixture was filtered and loaded directly on a reverse phase HPLC column (Column: Phenemonex Gemini NX C18, 150×21.2 mm, 5 μm, AXIA Pack, Water+10 mM Ammonium Acetate/Acetonitrile 10-45% over 12 min) to afford Example 192 (25.5 mg, 64%) as a white solid. LCMS m/z 600.9 (M+H)⁺; ¹H NMR (600 MHZ, DMSO-d₆) δ 11.88 (br. s, 1H), 8.05 (s, 1H), 8.00 (s, 1H), 7.35 (s, 1H), 6.69 (s, 2H), 5.76 (s, 1H), 4.97-4.82 (m, 1H), 3.85 (s, 3H), 3.73 (s, 6H), 3.11 (dd, J=2.5, 11.9 Hz, 1H), 2.77-2.72 (m, 1H), 2.33 (dt, J=3.7, 11.9 Hz, 1H), 1.92-1.64 (m, 8H), 0.94-0.88 (m, 2H), 0.67-0.63 (m, 2H); ¹⁹F NMR (565 MHZ, DMSO-d₆) δ −184.26 (s, 1F).

Synthesis of N-{6-[(5-cyclopropyl-1H-pyrazol-3-yl)amino]-5-methoxy-1,2-benzoxazol-3-yl}-4-trans (4-fluoro-1-methylpiperidin-2-yl)-2,6-dimethoxybenzene-1-sulfonamide (Example 193). Example 193 was made in a similar fashion as Example 192 starting with 192d in place of 192c for Step 4. Example 193 (70.3 mg, 59%) was isolated as a white solid. LCMS m/z 601.3 (M+H)⁺; ¹H NMR (600 MHZ, DMSO-d6) δ 8.06 (s, 1H), 8.01 (s, 1H), 7.36 (s, 1H), 6.70 (s, 2H), 5.76 (s, 1H), 4.95-4.84 (m, 1H), 3.86 (s, 3H), 3.74 (s, 6H), 3.12 (dd, J=2.6, 11.9 Hz, 1H), 2.77-2.72 (m, 1H), 2.33 (dt, J=3.7, 11.9 Hz, 1H), 1.91-1.65 (m, 8H), 0.93-0.89 (m, 2H), 0.67-0.64 (m, 2H); ¹⁹F NMR (565 MHZ, DMSO-d6) δ −184.29 (s, 1F).

### Example 194: N-{6-[(3-cyclopropyl-1H-pyrazol-5-yl)amino]-5-methoxy-1,2-benzoxazol-3-yl}-2,6-dimethoxy-4-[(4-methoxypiperidin-1-yl)methyl]benzene-1-sulfonamide

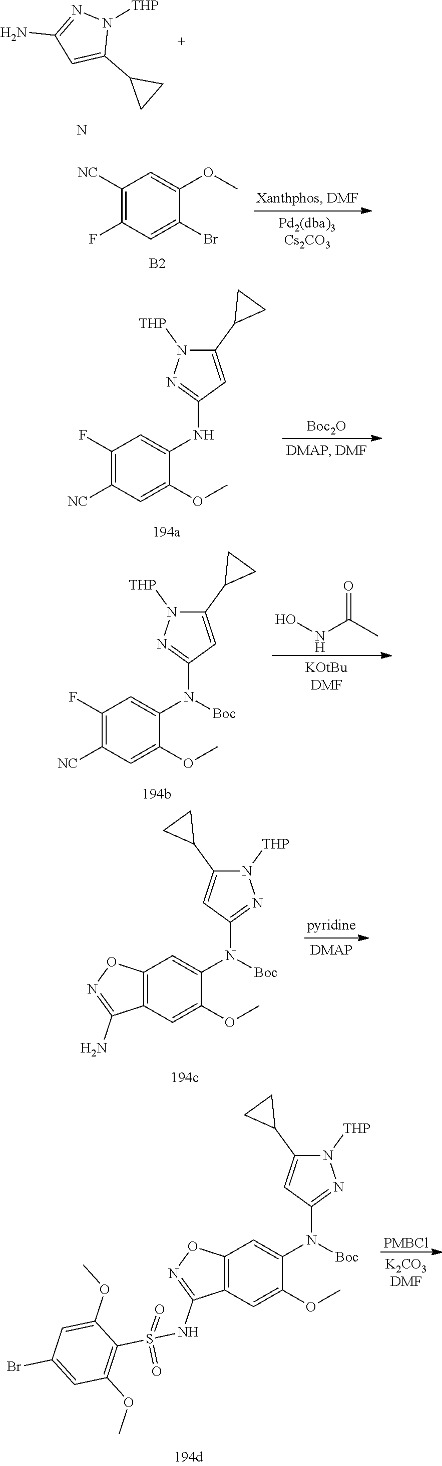

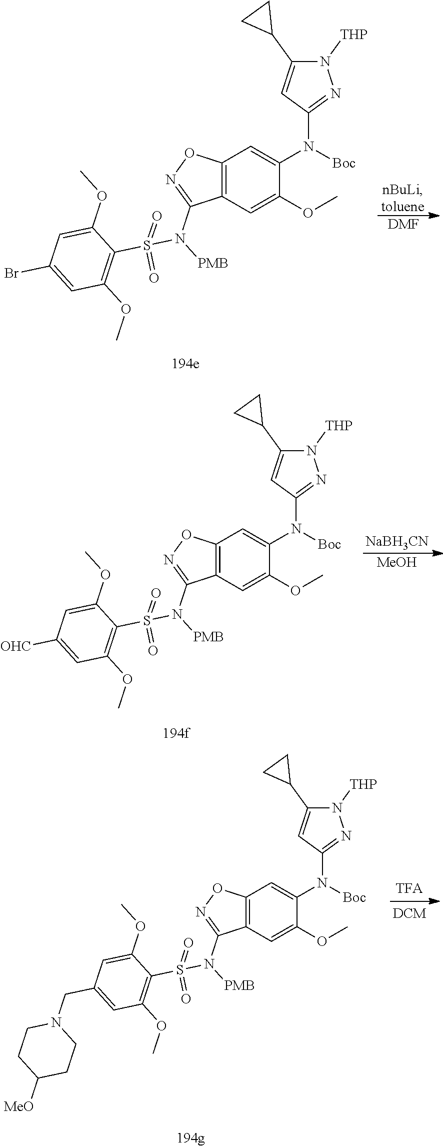

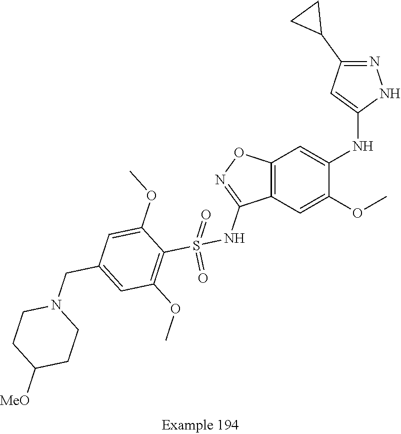 [SMILES: COc1cc2c(NS(=O)(=O)c3c(OC)cc(CN4CCC(OC)CC4)cc3OC)noc2cc1Nc1cc(C2CC2)n[nH]1]

### Step 1:4-{[5-cyclopropyl-1-(oxan-2-yl)-1H-pyrazol-3-yl]amino}-2-fluoro-5-methoxybenzonitrile (194a)

A mixture of 4-bromo-2-fluoro-5-methoxybenzonitrile B2 (186 g, 811 mmol), 5-cyclopropyl-1-(oxan-2-yl)-1H-pyrazol-3-amine Intermediate N (160 g, 772 mmol), and cesium carbonate (75.5 g, 2.32 mol) in DMF (1.6 L) was sparged with nitrogen for 5 mins. To the mixture was added Xantphos (40.2 g, 69.5 mmol) followed by Pd₂(dba)₃ (21.2 g, 23.2 mmol). The mixture was heated to 100° C. for 16 hrs, then cooled, filtered through a pad of silica gel and rinsed with DMF (10 vol.). The filtrate was stirred in an equal volume of water for 1 hr, and the resultant precipitate was collected via filtration and rinsed sequentially with 1:1 DMF/water (two vol.) followed by water (4 vol.). The resultant filter cake was dried under vacuum at 50° C. to afford 194a (222 g, 81%) as a white solid. ¹H NMR (400 MHZ, DMSO-d₆) d 8.71 (s, 1H), 8.13 (d, J=12.8 Hz, 1H), 7.27 (d, J=6 Hz, 1H), 5.79 (s, 1H), 5.52-5.50 (m, 1H), 4.02 (m, 4H), 3.74-3.65 (m, 1H), 2.35-2.26 (m, 1H), 2.10-2.01 (m, 1H), 1.98 (m, 2H), 1.40 (m, 1H), 1.50 (m, 2H), 0.90 (m, 2H), 0.70-0.50 (m, 2H).

### Step 2: tert-butyl (4-cyano-5-fluoro-2-methoxyphenyl) [5-cyclopropyl-1-(oxan-2-yl)-1H-pyrazol-3-yl]carbamate (194b)

To 4-{[5-cyclopropyl-1-(oxan-2-yl)-1H-pyrazol-3-yl]amino}-2-fluoro-5-methoxybenzonitrile 194a (45 g, 130 mmol), DMAP (2.62 g, 21.5 mmol), and di-tert-butyl dicarbonate (46.8 g, 215 mmol) was added DMF (450 mL). The mixture was heated at 80° C. for 16 hrs, then diluted with water (225 mL) and cooled to rt. After stirring at rt for 1 hr, the solids were collected via filtration and rinsed twice with two bed volumes of DMF/water (2:1). The filter cake was dried at 50° C. for 16 hrs to afford 194b (54.3 g, 94%) as a white solid.

### Step 3: tert-butyl (3-amino-5-methoxy-1,2-benzoxazol-6-yl) [5-cyclopropyl-1-(oxan-2-yl)-1H-pyrazol-3-yl]carbamate (194c)

To acetohydroxamic acid (59.2 g, 789 mmol) and potassium tert-butoxide (89.2 g, 789 mmol) was added DMF (600 mL). The mixture was stirred for 30 mins at rt, and was then charged with tert-butyl (4-cyano-5-fluoro-2-methoxyphenyl) [5-cyclopropyl-1-(oxan-2-yl)-1H-pyrazol-3-yl]carbamate 194b (60.0 g, 130 mmol) and additional DMF (600 mL). The mixture was brought to 80° C. for 1 hr and was then cooled to rt and filtered through celite. The filter cake was rinsed with DMF (5 vol.), and to the combined filtrate was added EtOAc (20 vol.) and water (20 vol.). The aqueous phase was extracted with EtOAc (2×10 vol.), and the combined organic extracts were concentrated under vacuum. The residue was passed through a silica column to afford 194c (35.0 g, 57%). ¹H NMR (400 MHZ, DMSO-d₆) d 7.44 (s, 1H), 7.28 (s, 1H), 6.33 (s, 2H), 6.05 (s, 1H), 5.39 (dd, J=2.0, 9.6 Hz, 1H), 3.88-3.78 (m, 4H), 3.60-3.51 (m, 2H), 2.30-2.01 (m, 6H), 1.32 (s, 9H), 0.95 (m, 2H), 0.06 (m, 2H).

### Step 4: tert-butyl [3-(4-bromo-2,6-dimethoxybenzene-1-sulfonamido)-5-methoxy-1,2-benzoxazol-6-yl][5-cyclopropyl-1-(oxan-2-yl)-1H-pyrazol-3-yl]carbamate (194d)

To tert-butyl (3-amino-5-methoxy-1,2-benzoxazol-6-yl) [5-cyclopropyl-1-(oxan-2-yl)-1H-pyrazol-3-yl]carbamate 194c (5.00 g, 10.6 mmol), 4-bromo-3,5-dimethoxybenzene-1-sulfonyl chloride (4.03 g, 12.8 mmol), and DMAP (130 mg, 1.06 mmol) was added pyridine. The mixture was capped and heated to 40° C. for 16 hrs. The mixture was partitioned between DCM and 1 M AcOH, and the aqueous layer was extracted three times with DCM. Combined extracts were washed with brine, dried over Na₂SO₄, and concentrated to afford 194d (7.97 g, 99%) as a yellow solid which was used directly in the next step. LCMS m/z 748, 750 (M+H)⁺.

### Step 5: tert-butyl (3-{(4-bromo-2,6-dimethoxybenzene-1-sulfonyl) [(4-methoxyphenyl)methyl]amino}-5-methoxy-1,2-benzoxazol-6-yl) [5-cyclopropyl-1-(oxan-2-yl)-1H-pyrazol-3-yl]carbamate (194e)

To a stirred mixture of tert-butyl [3-(4-bromo-2,6-dimethoxybenzene-1-sulfonamido)-5-methoxy-1,2-benzoxazol-6-yl][5-cyclopropyl-1-(oxan-2-yl)-1H-pyrazol-3-yl]carbamate 194d (7.97 g, 10.7 mmol), and potassium carbonate (5.89 g. 42.6 mmol) in DMF (106 mL) was added dropwise 4-methoxybenzyl chloride (2.50 g, 16.0 mmol), and the mixture was brought to 80° C. and stirred at that temperature for 3 hrs. To the mixture was added additional 4-methoxybenzyl chloride (600 g, 3.83 mmol). After heating for an additional 3 hrs, the mixture was cooled to rt, diluted with water (100 mL), and stirred for 10 mins. The mixture was extracted with EtOAc (3×100 mL) and the combined extracts were washed with brine, dried over sodium sulfate and concentrated. Silica gel chromatography eluting with a gradient of DCM/MeOH (0-5% MeOH) afforded 194e (8.26 g, 89%) as a yellow solid. LCMS m/z 868, 870 (M+H)⁺.

### Step 6: tert-butyl [5-cyclopropyl-1-(oxan-2-yl)-1H-pyrazol-3-yl](3-{(4-formyl-2,6-dimethoxybenzene-1-sulfonyl)[(4-methoxyphenyl)methyl]amino}-5-methoxy-1,2-benzoxazol-6-yl) carbamate (194f)

To tert-butyl (3-{(4-bromo-2,6-dimethoxybenzene-1-sulfonyl) [(4-methoxyphenyl)methyl]amino}-5-methoxy-1,2-benzoxazol-6-yl) [5-cyclopropyl-1-(oxan-2-yl)-1H-pyrazol-3-yl]carbamate 194e (4.00 g, 4.64 mmol) in toluene (96 mL) at −70° C. was added dropwise over 15 mins 2.5 M n-butyllithium in hexanes (590 mg, 9.21 mmol, 3.68 mL). After stirring for 30 mins, DMF (2.02 g, 27.6 mmol) was added dropwise. The resultant mixture was stirred at −70° C. for 45 mins and was then allowed to warm to −50° C., at which point it was quenched with sat. ammonium chloride. The mixture was brought to rt and partitioned between water and EtOAc. The aqueous portion was extracted with EtOAc (3×100 mL) and the combined extracts were washed with brine, dried over sodium sulfate, and concentrated to afford 194f (1.0 g, 27%) as a yellow solid. ¹H NMR (DMSO-d₆, 400 MHz) d 10.01 (s, 1H), 7.50-7.50 (m, 1H), 7.32 (s, 2H), 7.31 (d, 1H, J=2.3 Hz), 7.20-7.10 (m, 1H), 6.85 (d, 2H, J=8.1 Hz), 6.80 (d, 1H, J=8.6 Hz), 6.03 (br. s, 1H), 5.38 (dd, 1H, J=2.1, 9.6 Hz), 5.02 (br. d, 2H, J=9.4 Hz), 3.81 (s, 6H), 3.70 (s, 2H), 3.69 (s, 3H), 3.66 (d, 3H, J=3.4 Hz), 1.90-1.90 (m, 2H), 1.90-1.80 (m, 1H), 1.70-1.60 (m, 1H), 1.60-1.50 (m, 1H), 1.42 (br. s, 2H), 1.28 (br. s, 9H), 1.0-0.90 (m, 2H), 0.65 (br. dd, 2H, J=4.9, 7.6 Hz); LCMS m/z 818 (M+H)⁺.

### Step 7: tert-butyl [5-cyclopropyl-1-(oxan-2-yl)-1H-pyrazol-3-yl][3-({2,6-dimethoxy-4-[(4-methoxypiperidin-1-yl)methyl]benzene-1-sulfonyl}[(4-methoxyphenyl)methyl]amino)-5-methoxy-1,2-benzoxazol-6-yl]carbamate (194 g)

To 4-methoxypiperidine (18.6 mg, 0.161 mmol) in MeOH (1.0 mL) was added tert-butyl [5-cyclopropyl-1-(oxan-2-yl)-1H-pyrazol-3-yl](3-{(4-formyl-2,6-dimethoxybenzene-1-sulfonyl)[(4-methoxyphenyl)methyl]amino}-5-methoxy-1,2-benzoxazol-6-yl) carbamate 194f (110 Mg, 0.134 mmol). After stirring at rt for 20 mins, sodium cyanoborohydride was added (25.4 mg, 0.403 mmol). The mixture was stirred for 15 mins, then filtered and concentrated to afford 194 g (123 mg, 99%) as a yellow solid, which was used in the next step without further purification. LCMS m/z 818.8 (M+H−Boc)⁺.

Step 8: N-{6-[(3-cyclopropyl-1H-pyrazol-5-yl)amino]-5-methoxy-1,2-benzoxazol-3-yl}-2,6-dimethoxy-4-[(4-methoxypiperidin-1-yl)methyl]benzene-1-sulfonamide (Example 194) To a solution of tert-butyl [5-cyclopropyl-1-(oxan-2-yl)-1H-pyrazol-3-yl][3-({2,6-dimethoxy-4-[(4-methoxypiperidin-1-yl)methyl]benzene-1-sulfonyl}[(4-methoxyphenyl)methyl]amino)-5-methoxy-1,2-benzoxazol-6-yl]carbamate 194 g (120 mg, 0.131 mmol) in DCM (2.0 mL) was added TFA (1.0 mL) The solution was stirred for 16 hrs, and concentrated. Purification via preparative HPLC (C18 150×30 mm column, ammonium carbonate/H₂O, ACN, gradient of 19-39% ACN over 9 mins, 30 mL/mins) to afford Example 194 (6.01 mg, 3.2%) as a white solid. ¹H NMR (400 MHZ, DMSO-d₆) δ 11.95-11.86 (m, 1H), 10.93 (ddd, J=3.0, 6.8, 11.8 Hz, 1H), 8.12 (s, 1H), 8.06 (s, 1H), 7.39 (s, 1H), 6.66 (s, 2H), 5.77 (s, 1H), 3.86 (s, 3H), 3.74 (s, 6H), 3.41 (br s, 3H), 3.29-3.11 (m, 4H), 2.64-2.54 (m, 2H), 2.12-2.01 (m, 2H), 1.90-1.75 (m, 3H), 1.48-1.35 (m, 2H), 0.95-0.87 (m, 2H), 0.69-0.62 (m, 2H); LCMS m/z 613.4 (M+H)⁺.

The Examples in Table 20 were made in a similar fashion as Example 194 using commercially available amines for the reductive amination reaction (see step 7).

TABLE 20

| Example | | |
|---|---|---|
| Number | Structure/IUPAC Name | Analytical Data |
| 195 | 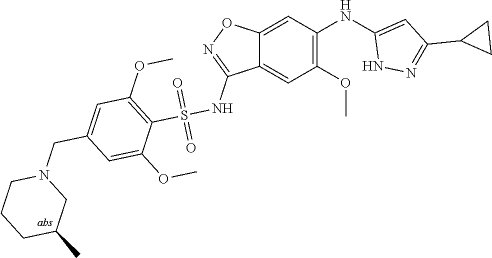 [SMILES: COc1cc2c(NS(=O)(=O)c3c(OC)cc(CN4CCC[C@H](C)C4)cc3OC)noc2cc1Nc1cc(C2CC2)n[nH]1] | *AcOH was used as an additive. ¹H NMR (MeOD, 400 MHz) δ 7.67 (br. s, 1H), 7.34 (s, 1H), 6.81 (s, 2H), 5.77 (s, 1H), 3.99 (s, 3H), 3.89 (br. s, 2H), 3.87 (s, 6H), 3.2-2.9 (m, 3H), 2.5-2.4 (m, 1H), 2.16 (br. d, 1H, J = 1.3 Hz), 2.0-1.9 (m, 1H), 1.8-1.7 (m, 3H), 1.68 (br. s, 1H), 1.0-1.0 (m, 2H), 0.93 (d, 3H, J = 6.5 Hz), 0.8-0.7 (m, 2H); LCMS m/z 597.3 (M + H)⁺. |
| | N-{6-[(3-cyclopropyl-1H-pyrazol-5- | |
| | yl)amino]-5-methoxy-1,2-benzoxazol- | |
| | 3-yl}-2,6-dimethoxy-4-{[(3S)-3- | |
| | methylpiperidin-1-yl]methyl}benzene- | |
| | 1-sulfonamide | |
| 196 | 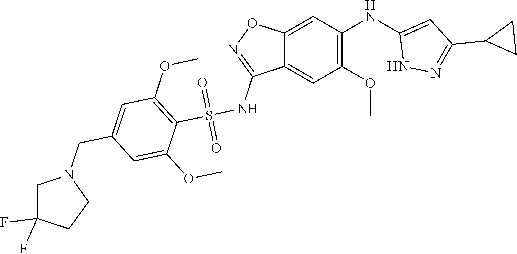 [SMILES: COc1cc2c(NS(=O)(=O)c3c(OC)cc(CN4CCC(F)(F)C4)cc3OC)noc2cc1Nc1cc(C2CC2)n[nH]1] | ¹H NMR (400 MHz, DMSO-d₆) δ 11.91 (br. s, 1H) 11.09-10.88 (m, 1 H) 8.10 (br. d, J = 19.59 Hz, 2H) 7.39 (s, 1H) 6.67 (s, 2H) 5.77 (s, 1H) 3.86 (s, 3H) 3.75 (s, 6H) 3.58 (s, 2H) 2.86 (t, J = 13.31 Hz, 2H) 2.72-2.64 (m, 2H) 2.32-2.16 (m, 2H) 1.91-1.79 (m, 1H) 0.95-0.87 (m, 2H) 0.69-0.62 (m, 2H); LCMS m/z 605.3 (M + H)⁺. |
| | N-{6-[(3-cyclopropyl-1H-pyrazol-5- | |
| | yl)amino]-5-methoxy-1,2-benzoxazol- | |
| | 3-yl}-4-[(3,3-difluoropyrrolidin-1- | |
| | yl)methyl]-2,6-dimethoxybenzene-1- | |
| | sulfonamide | |
| 197 | 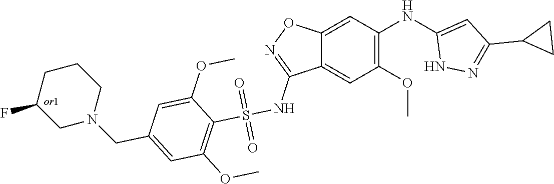 [SMILES: COc1cc2c(NS(=O)(=O)c3c(OC)cc(CN4CCC[C@H](F)C4)cc3OC)noc2cc1Nc1cc(C2CC2)n[nH]1] | ¹H NMR (600 MHz, DMSO-d₆) δ 11.98-11.78 (m, 1H), 8.11-7.88 (m, 2H), 7.32 (s, 1H), 6.64 (s, 2H), 5.75 (s, 1H), 4.71-4.54 (m, 1H), 3.84 (s, 3H), 3.70 (s, 6H), 3.62-3.57 (m, 2H), 2.66-2.57 (m, 1H), 2.40-2.31 (m, 2H), 2.23 (br t, J = 8.0 Hz, 1H), 1.85 (tt, J = 5.0, 8.5 Hz, 1H), 1.81- 1.65 (m, 2H), 1.52 (dt, J = 3.4, 7.9 Hz, 1H), 1.45 (ddd, J = 4.3, 8.3, 12.6 Hz, 1H), 0.94-0.88 (m, 2H), 0.69- 0.61 (m, 2H); LCMS m/z 601.0 (M + H)⁺ |
| | Or 1 = single enantiomer | |
| | stereochemistry unknown | |
| | N-{6-[(3-cyclopropyl-1H-pyrazol-5- | |
| | yl)amino]-5-methoxy-1,2-benzoxazol- | |
| | 3-yl}-4-[(3-fluoropiperidin-1-yl)methyl]- | |
| | 2,6-dimethoxybenzene-1-sulfonamide | |
| 198 | 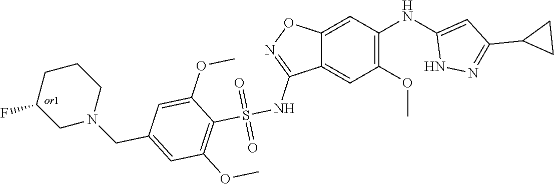 [SMILES: COc1cc2c(NS(=O)(=O)c3c(OC)cc(CN4CCC[C@@H](F)C4)cc3OC)noc2cc1Nc1cc(C2CC2)n[nH]1] | ¹H NMR (600 MHz, DMSO-d₆) δ 11.98-11.78 (m, 1H), 8.10-7.86 (m, 2H), 7.31 (s, 1H), 6.64 (s, 2H), 5.75 (s, 1H), 4.71-4.55 (m, 1H), 3.84 (s, 3H), 3.70 (s, 6H), 3.59 (br d, J = 18.4 Hz, 2H), 2.66-2.58 (m, 1H), 2.40-2.32 (m, 2H), 2.23 (br t, J = 8.1 Hz, 1H), 1.85 (tt, J = 5.1, 8.4 Hz, 1H), 1.82-1.66 (m, 2H), 1.52 (dt, J = 3.6, 8.1 Hz, 1H), 1.48-1.41 (m, 1H), 0.94-0.87 (m, 2H), 0.68-0.63 (m, 2H); LCMS m/z 601.0 (M + H)⁺ |
| | Or 1 = single enantiomer | |
| | stereochemistry unknown | |
| | N-{6-[(3-cyclopropyl-1H-pyrazol-5- | |
| | yl)amino]-5-methoxy-1,2-benzoxazol- | |
| | 3-yl}-4-[(3-fluoropiperidin-1-yl)methyl]- | |
| | 2,6-dimethoxybenzene-1-sulfonamide | |
| 199 | 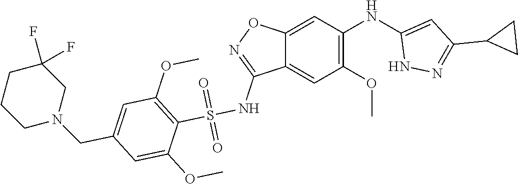 [SMILES: COc1cc2c(NS(=O)(=O)c3c(OC)cc(CN4CCCC(F)(F)C4)cc3OC)noc2cc1Nc1cc(C2CC2)n[nH]1] | ¹H NMR (400 MHz, DMSO-d₆) δ 11.02 (s, 1H) 8.09 (s, 2H) 7.39 (s, 1H) 7.24-6.94 (m, 1H) 6.75 (br. s, 2H) 5.77 (s, 1H) 3.87 (s, 3H) 3.76 (s, 6H) 3.55 (br. s, 2H) 3.04-2.84 (m, 2H) 2.73-2.57 (m, 2H) 2.01- 1.90 (m, 2H) 1.88-1.82 (m, 1H) 1.73 (br. d, J = 1.63 Hz, 2H) 0.96- 0.84 (m, 2H) 0.68-0.61 (m, 2H); LCMS m/z 619.3 (M + H)⁺. |
| | N-{6-[(3-cyclopropyl-1H-pyrazol-5- | |
| | yl)amino]-5-methoxy-1,2-benzoxazol- | |
| | 3-yl}-4-[(3,3-difluoropiperidin-1- | |
| | yl)methyl]-2,6-dimethoxybenzene-1- | |
| | sulfonamide | |

### Example 200: N-{6-[(3-cyclopropyl-1H-pyrazol-5-yl)amino]-5-methoxy-1,2-benzoxazol-3-yl}-2,6-dimethoxy-4-[(3-methylazetidin-1-yl)methyl benzene-1-sulfonamide

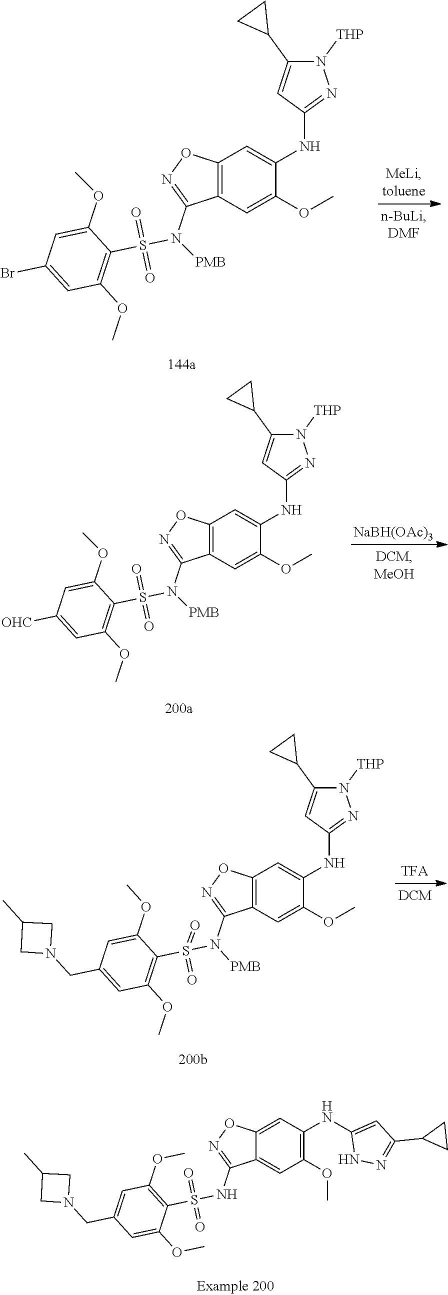

### Step 1: Synthesis of N-(6-{[5-cyclopropyl-1-(oxan-2-yl)-1H-pyrazol-3-yl]amino}-5-methoxy-1,2-benzoxazol-3-yl)-4-formyl-2,6-dimethoxy-N-[(4-methoxyphenyl)methyl]benzene-1-sulfonamide (200a)

To a-70° C. solution of 144a (2200 mg, 2.862 mmol) in toluene (59.6 mL) was added methyllithium (189 mg, 8.59 mmol, 5.27 mL) dropwise via syringe under a nitrogen atmosphere over 15 mins. After stirring at −70° C. for 0.5 hrs, n-butyllithium (367 mg, 5.72 mmol, 3.39 mL) was added dropwise via syringe and stirring was continued at −70° C. for another 0.5 hrs then DMF (1260 mg, 17.2 mmol) was added dropwise via syringe. The resulting mixture was stirred at −70° C. for 45 mins. The reaction was quenched with sat. aq. NH₄Cl (60 mL) at −50° C. and allowed to warm to rt. The mixture was partitioned between water (100 mL) and EtOAc (180 mL). The aqueous layer was extracted with EtOAc (100 mL) and the combined organic layers were washed with brine (120 mL), dried over anhydrous Na₂SO₄, filtered and concentrated. The crude residue was purified by column chromatography (28-35% EtOAc in pet. ether) to give 200a (1.38 g, 64%) as a yellow solid. LCMS m/z 773.3 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-[(3-methylazetidin-1-yl)methyl]benzene-1-sulfonamide (200b)

To a suspension of 200a (80.0 mg, 0.11 mmol) in DCM (0.7 mL) and MeOH (0.7 mL) was added 3-methylazetidine hydrochloride (14.4 mg, 0.134 mmol). The mixture was stirred at rt for 40 mins then STAB (70.9 mg, 0.334 mmol) was added, and the reaction was stirred for 1 hr. The crude reaction was diluted with H₂O (10 mL) and extracted with DCM (10×3 mL). The combined organic extracts were dried over Na₂SO₄, filtered and concentrated to afford crude 200b (85 mg) as yellow glass. LCMS m/z 773.3 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-[(3-methylazetidin-1-yl)methyl]benzene-1-sulfonamide (Example 200)

A solution 200b (85 mg, 0.11 mmol) in DCM (2 mL) and TFA (4 mL) was stirred at rt for 3 hrs. The reaction was concentrated and purified by reverse phase HPLC (8-48% acetonitrile/water with NH₄OH; 60 mL/min flow rate in 9 mins, Phenomenex Gemini NX 150×30 mm, 5 μm) to afford Example 200 (14 mg, 23%) as a white solid. ¹H NMR (400 MHZ, DMSO-d₆) δ 11.90 (s, 1H) 10.92 (br s, 1H) 8.15-8.01 (m, 2H) 7.39 (s, 1H) 6.61 (s, 2H) 5.78 (s, 1H) 3.86 (s, 3H) 3.74 (s, 6H) 3.50 (s, 2H) 3.38 (br. t, J=7.19 Hz, 2H) 2.74-2.64 (m, 2H) 2.33 (br. s, 1H) 1.89-1.81 (m, 1H) 1.09 (d, J=6.63 Hz, 3H) 0.95-0.86 (m, 2H) 0.70-0.60 (m, 2H); LCMS m/z 569.4 (M+H)⁺.

The Examples in Table 21 were made in a similar fashion as Example 200 using commercially available amines for the reductive amination reaction (see step 2)

TABLE 21

| Example | | |
|---|---|---|
| Number | Structure/IUPAC Name | Analytical Data |
| 201 | 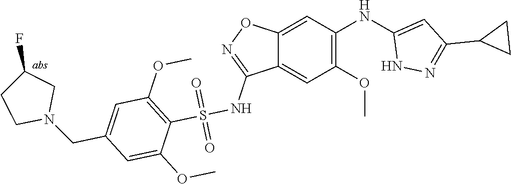 [SMILES: COc1cc2c(NS(=O)(=O)c3c(OC)cc(CN4CC[C@@H](F)C4)cc3OC)noc2cc1Nc1cc(C2CC2)n[nH]1] | ¹H NMR (400 MHz, DMSO-d₆) δ 11.90 (s, 1H) 10.93 (br. s, 1H) 8.12 (s, 1H) 8.05 (s, 1H) 7.39 (s, 1H) 6.68 (s, 2H) 5.78 (s, 1H) 5.29-5.08 (m, 1H) 3.87 (s, 3H) 3.75 (s, 6H) 3.58 (br. s, 2H) 2.83-2.70 (m, 2H) 2.68-2.55 (m, 1H) 2.39-2.32 (m, 1H) 2.20-2.07 (m, 1H) 1.95-1.81 (m, 2H) 0.95-0.86 (m, 2H) 0.70- 0.62 (m, 2H); LCMS m/z 587.3 (M + H)⁺. |
| | N-{6-[(3-cyclopropyl-1H-pyrazol-5- | |
| | yl)amino]-5-methoxy-1,2-benzoxazol- | |
| | 3-yl}-4-{[(3R)-3-fluoropyrrolidin-1- | |
| | yl]methyl}-2,6-dimethoxybenzene-1- | |
| | sulfonamide | |
| 202 | 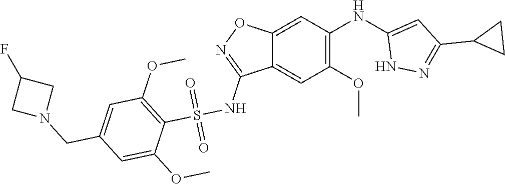 [SMILES: COc1cc2c(NS(=O)(=O)c3c(OC)cc(CN4CC(F)C4)cc3OC)noc2cc1Nc1cc(C2CC2)n[nH]1] | ¹H NMR (400 MHz, DMSO-d₆) δ 12.03-11.63 (m, 1H), 11.20-10.78 (m, 1H), 8.12-8.06 (m, 1H), 8.03 (s, 1H), 7.39 (s, 1H), 6.62 (s, 2H), 5.78 (s, 1H), 5.27-5.07 (m, 1H), 3.86 (s, 3H), 3.74 (s, 6H), 3.60 (s, 2H), 3.59-3.52 (m, 2H), 3.15 (td, J = 4.8, 19.4 Hz, 2H), 1.89-1.82 (m, 1H), 0.94-0.88 (m,2H), 0.68-0.63 (m, 2H); LCMS m/z 573.3 (M + H)⁺. |
| | N-{6-[(3-cyclopropyl-1H-pyrazol-5- | |
| | yl)amino]-5-methoxy-1,2-benzoxazol- | |
| | 3-yl}-4-[(3-fluoroazetidin-1-yl)methyl]- | |
| | 2,6-dimethoxybenzene-1-sulfonamide | |
| 203 | 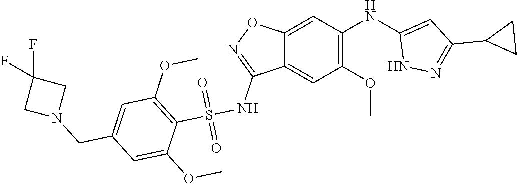 [SMILES: COc1cc2c(NS(=O)(=O)c3c(OC)cc(CN4CC(F)(F)C4)cc3OC)noc2cc1Nc1cc(C2CC2)n[nH]1] | *TEA and Dichloroethane were used in place of DCM and MeOH ¹H NMR (400 MHz, DMSO-d₆) δ 11.90 (br. s, 1H), 10.95 (br. s, 1H), 8.22-7.89 (m, 2H), 7.40 (s, 1H), 6.65 (s, 2H), 5.78 (s, 1H), 3.87 (s, 3H), 3.75 (s, 6H), 3.70 (s, 2H), 3.63 (t, J = 12.6 Hz, 4H), 1.91-1.80 (m, 1H), 0.97-0.86 (m, 2H), 0.70-0.61 (m, 2H); LCMS m/z 591.2 (M + H)⁺. |
| | N-{6-[(3-cyclopropyl-1H-pyrazol-5- | |
| | yl)amino]-5-methoxy-1,2-benzoxazol- | |
| | 3-yl}-4-[(3,3-difluoroazetidin-1- | |
| | yl)methyl]-2,6-dimethoxybenzene-1- | |
| | sulfonamide | |

### Example 204: N-{6-[(3-cyclopropyl-1H-pyrazol-5-yl)amino]-5-methoxy-1,2-benzoxazol-3-yl}-4-{[(3R)-3-(difluoromethyl)pyrrolidin-1-yl]methyl}-2,6-dimethoxybenzene-1-sulfonamide

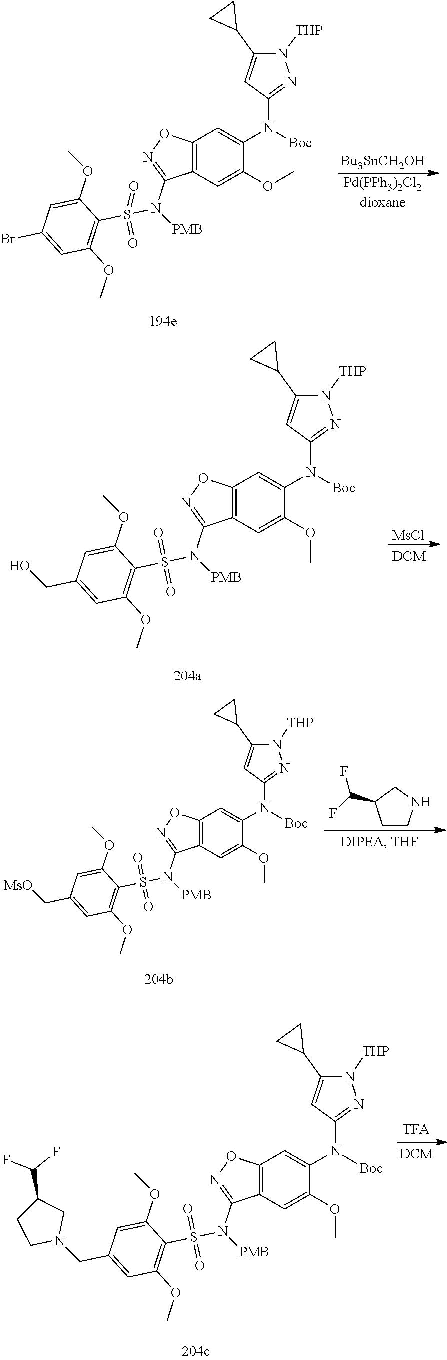

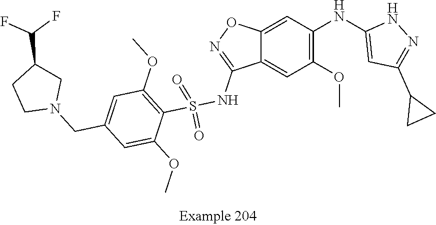 [SMILES: COc1cc2c(NS(=O)(=O)c3c(OC)cc(CN4CC[C@@H](C(F)F)C4)cc3OC)noc2cc1Nc1cc(C2CC2)n[nH]1]

### Step 1: Synthesis of tert-butyl [5-cyclopropyl-1-(oxan-2-yl)-1H-pyrazol-3-yl](3-{[4-(hydroxymethyl)-2,6-dimethoxybenzene-1-sulfonyl][(4-methoxyphenyl)methyl]amino]-5-methoxy-1,2-benzoxazol-6-yl)carbamate (204a)

A solution of 194e (2350 mg, 2.705 mmol and (tributylstannyl) methanol (1300 mg, 4.06 mmol) in dioxane (22.5 mL) was added Pd(PPh₃)₂Cl₂ (94.9 mg, 0.135 mmol). The reaction was stirred at 80° C. for 16 hrs under nitrogen. The solvent was removed, and the crude residue was purified by column chromatography (ISCO, SiO₂, 0-6% MeOH/DCM) to give 204a (2200 mg, 85%) as yellow solid. LCMS m/z 820.4 (M+H)⁺.

### Step 2: Synthesis of (4-{(6-{(tert-butoxycarbonyl)[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)methyl methanesulfonate (204b)

To a solution of 204a (2200 mg, 2.683 mmol) in DCM (22 mL) was added methyl morpholine (407 mg, 4.02 mmol) and methanesulfonyl chloride (1030 mg, 8.99 mmol) at 25° C. The reaction was stirred at 25° C. for 0.5 hrs. The crude reaction was diluted with DCM and water. The layers were separated, and the organic layer was washed with brine, then dried (Na₂SO₄), filtered and concentrated to give 204b (2400 mg, 99%) as a yellow solid. LCMS m/z 898.2 (M+H)⁺.

### Step 3: Synthesis of tert-butyl [5-cyclopropyl-1-(oxan-2-yl)-1H-pyrazol-3-yl](3-{(4-{[(3R)-3-(difluoromethyl)pyrrolidin-1-yl]methyl}-2,6-dimethoxybenzene-1-sulfonyl)[(4-methoxyphenyl)methyl]amino}-5-methoxy-1,2-benzoxazol-6-yl) carbamate (204c)

To a solution of 204b (100 mg, 0.089 mmol) and (3R)-3-(difluoromethyl)pyrrolidine (70.2 mg, 0.445 mmol) in THF (2.0 mL) was added DIPEA (173 mg, 1.34 mmol) at 25° C. The reaction was stirred at 25° C. for 16 hrs then concentrated to give crude 204c (100 mg, >99%) as yellow gum, which was used to the next step directly.

### Step 4: Synthesis of N-{6-[(3-cyclopropyl-1H-pyrazol-5-yl)amino]-5-methoxy-1,2-benzoxazol-3-yl}-4-{[(3R)-3-(difluoromethyl)pyrrolidin-1-yl]methyl}-2,6-dimethoxybenzene-1-sulfonamide (Example 204)

To a solution of crude 204c (100 mg, 0.108 mmol) in DCM (2 mL) was added TFA (1 mL) at 25° C. The reaction was stirred at 25° C. for 16 hrs. The crude reaction was concentrated and purified by reverse phase HPLC (23-43% acetonitrile/water plus 10 mM ammonium acetate; 35 mL/min flow rate in 10 mins; Boston Prime-C18 150×30 mm, 5 μm) to afford Example 204 (17 mg, 25%) as a white powder. ¹H NMR (400 MHZ, METHANOL-d₄) δ 7.74-7.56 (m, 1H), 7.37-7.28 (m, 1H), 6.77-6.69 (m, 2H), 5.98-5.60 (m, 2H), 4.01-3.94 (m, 3H), 3.89-3.80 (m, 6H), 3.68-3.54 (m, 2H), 2.72-2.50 (m, 5H), 2.02-1.87 (m, 2H), 1.87-1.74 (m, 1H), 1.03-0.95 (m, 2H), 0.79-0.71 (m, 2H); LCMS m/z 619.3 (M+H)⁺.

The Examples in Table 22 were made in a similar fashion as Example 204 using commercially available amines for the mesylate displacement (see step 3)

TABLE 22

| Example | | |
|---|---|---|
| Number | Structure/IUPAC Name | Analytical Data |
| 205 | 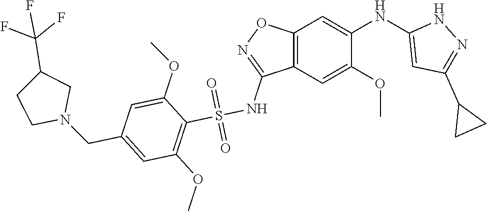 [SMILES: COc1cc2c(NS(=O)(=O)c3c(OC)cc(CN4CCC(C(F)(F)F)C4)cc3OC)noc2cc1Nc1cc(C2CC2)n[nH]1] | ¹H NMR (DMSO-d₆, 400 MHz) δ 11.91 (br. d, J = 1.1 Hz, 1H), 8.0-8.1 (m, 2H), 7.39 (s, 1H), 6.68 (s, 2H), 5.78 (d, J = 1.7 Hz, 1H), 3.87 (s, 3H), 3.74 (s, 6H), 3.5-3.7 (m, 2H), 3.0-3.2 (m, 1H), 2.68 (t, J = 9.2 Hz, 1H), 2.5-2.6 (m, 3H), 2.0-2.1 (m, 1H), 1.7-1.9 (m, 2H), 0.9-1.0 (m, 2H), 0.6-0.7 (m, 2H); LCMS m/z 637.3 (M + H)⁺. |
| | N-{6-[(3-cyclopropyl-1H-pyrazol-5- | |
| | yl)amino]-5-methoxy-1,2-benzoxazol- | |
| | 3-yl}-2,6-dimethoxy-4-{[3- | |
| | (trifluoromethyl)pyrrolidin-1- | |
| | yl]methyl}benzene-1-sulfonamide | |
| 206 | 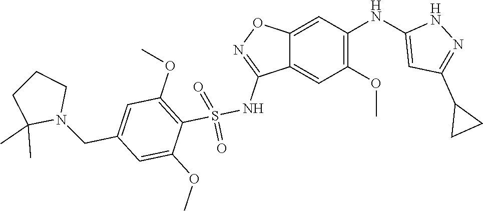 [SMILES: COc1cc2c(NS(=O)(=O)c3c(OC)cc(CN4CCCC4(C)C)cc3OC)noc2cc1Nc1cc(C2CC2)n[nH]1] | ¹H NMR (METHANOL-d₄, 400 MHz) δ 7.60 (br s, 1H), 7.31 (s, 1H), 6.73 (s, 2H), 5.75 (s, 1H), 3.95 (s, 3H), 3.81 (s, 6H), 3.66 (s, 2H), 2.77 (br d, J = 5.5 Hz, 2H), 1.89 (dt, J = 4.3, 8.9 Hz, 1H), 1.77 (br s, 4H), 1.16 (s, 6H), 1.02-0.91 (m, 2H), 0.77-0.69 (m, 2H); LCMS m/z 597.3 (M + H)⁺. |
| | N-{6-[(3-cyclopropyl-1H-pyrazol-5- | |
| | yl)amino]-5-methoxy-1,2-benzoxazol- | |
| | 3-yl}-4-[(2,2-dimethylpyrrolidin-1- | |
| | yl)methyl]-2,6-dimethoxybenzene-1- | |
| | sulfonamide | |

### Examples 207 and 208: N-{6-[(3-cyclopropyl-1H-pyrazol-5-yl)amino]-5-methoxy-1,2-benzoxazol-3-yl}-2,6-dimethoxy-4-[1-(morpholin-4-yl)ethyl]benzne-1-sulfonamide

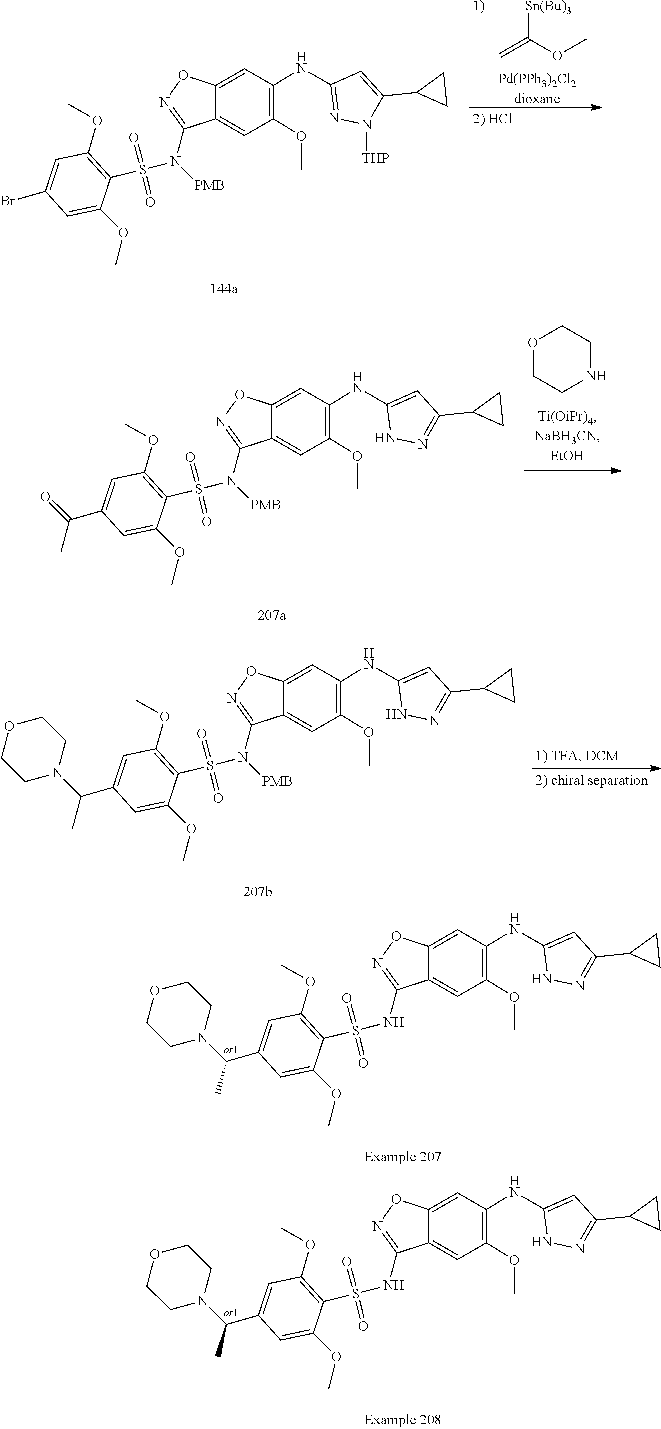

### Step 1: Synthesis of 4-acetyl-N-{6-[(3-cyclopropyl-1H-pyrazol-5-yl)amino]-5-methoxy-1,2-benzoxazol-3-yl}-2,6-dimethoxy-N-[(4-methoxyphenyl)methyl]benzene-1-sulfonamide (207a)

To a solution of 144a (1000 mg, 1.151 mmol) and tributyl (1-ethoxyvinyl) tin (540 mg, 1.5 mmol) in dioxane (9.59 mL) under nitrogen was added bis(triphenylphosphine) palladium chloride (40.4 mg, 0.0576 mmol). The reaction mixture was stirred at 100° C. for 3 hrs. HCl (2 N, 1.5 mL) was then added, and the mixture was stirred at 50° C. for 16 hrs. The reaction mixture was added to ice water (30 mL) and the resulting precipitate was filtered. The filter cake was washed with ice water (10 mL×3) and was then dried to give crude 207a (750 mg, >99%) as yellow solid. LCMS m/z 648.1 (M+H)⁺

### Step 2: Synthesis of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[1-(morpholin-4-yl)ethyl]benzene-1-sulfonamide (207b)

A solution of 4-acetyl-N-{6-[(3-cyclopropyl-1H-pyrazol-5-yl)amino]-5-methoxy-1,2-benzoxazol-3-yl}-2,6-dimethoxy-N-[(4-methoxyphenyl)methyl]benzene-1-sulfonamide 207a (500 mg, 0.654 mmol) and morpholine (171 mg, 1.96 mmol) in titanium (IV) isopropoxide (5 mL) was stirred at 60° C. for 16 hrs. Sodium cyanoborohydride (370 mg, 5.88 mmol) in EtOH (10 mL) was then added dropwise and the reaction mixture was stirred at 25° C. for 16 hrs. The resulting mixture was quenched with a sat. aq. solution of NaHCO₃ and filtered. The filtrate was extracted three times with DCM and the organic phase was dried and concentrated under reduced pressure. The resulting crude 207b (450 mg, >96%) was used in the next step without further purification. LCMS m/z 719.2 (M+H)⁺

### Step 3: Synthesis of N-{6-[(3-cyclopropyl-1H-pyrazol-5-yl)amino]-5-methoxy-1,2-benzoxazol-3-yl}-2,6-dimethoxy-4-[1-(morpholin-4-yl)ethyl]benzene-1-sulfonamide (Example 207 and 208)

To a solution of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[1-(morpholin-4-yl)ethyl]benzene-1-sulfonamide 207b (500 mg, 0.696 mmol) in DCM (10 mL) was added TFA (5 mL) at 25° C. The reaction mixture was stirred at 25° C. for 16 hrs and was then concentrated under reduced pressure. The crude residue was diluted with DMF (2.5 mL) and purified by preparative HPLC (C18 150×40 mm column, Mobile phase A: H₂O+NH₃H₂O+NH₄HCO₃ Mobile phase B: ACN 10-50% over 9 min, Flow rate 60 mL/min) to afford a racemic mixture of 207 and 208 (200 mg, 48%) as a white solid; LCMS m/z 599.1; 599.2 (M+H)⁺. The enantiomers were separated by chiral SFC (40% methanol with 0.1% ammonium hydroxide in CO₂; 150 mL/min flow rate; Column: Daicel Chiralpak® IH (250×30 mm; 10 □m))

### N-{6-[(3-cyclopropyl-1H-pyrazol-5-yl)amino]-5-methoxy-1,2-benzoxazol-3-yl}-2,6-dimethoxy-4-[1-(morpholin-4-yl)ethyl]benzene-1-sulfonamide (Example 207)

The first eluting peak was lyophilized and gave 207 (49.4 mg, 24.7%) as a white solid. ¹H NMR (400 MHZ, DMSO-d₆) δ 12.0-11.8 (m, 1H), 11.0-10.9 (m, 1H), 8.13 (s, 1H), 8.07 (s, 1H), 7.39 (s, 1H), 6.68 (s, 2H), 5.8-5.8 (m, 1H), 3.87 (s, 3H), 3.76 (s, 6H), 3.55 (br. s, 5H), 2.4-2.3 (m, 2H), 2.3-2.3 (m, 2H), 1.9-1.8 (m, 1H), 1.24 (br. d, J=6.4 Hz, 3H), 1.0-0.9 (m, 2H), 0.7-0.6 (m, 2H); LCMS m/z 599.3 (M+H)⁺

### N-{6-[(3-cyclopropyl-1H-pyrazol-5-yl)amino]-5-methoxy-1,2-benzoxazol-3-yl}-2,6-dimethoxy-4-[1-(morpholin-4-yl)ethyl]benzene-1-sulfonamide (Example 208)

The second eluting peak was lyophilized and gave 208 (41.1 mg, 20.6%) as a white solid. ¹H NMR (400 MHZ, DMSO-d₆) δ 12.0-11.8 (m, 1H), 11.0-10.9 (m, 1H), 8.2-8.1 (m, 1H), 8.1-8.0 (m, 1H), 7.4-7.4 (m, 1H), 6.67 (s, 2H), 5.8-5.8 (m, 1H), 3.87 (s, 3H), 3.76 (s, 6H), 3.6-3.5 (m, 5H), 2.37 (br. s, 2H), 2.23 (br. s, 2H), 1.9-1.8 (m, 1H), 1.24 (d, J=6.5 Hz, 3H), 0.92 (br. dd, J=2.1, 8.2 Hz, 2H), 0.66 (br. dd, J=2.1, 4.9 Hz, 2H); LCMS m/z 599.3 (M+H)⁺

### Example 209 and 210: N-{6-[(3-cyclopropyl-1H-pyrazol-5-yl)amino]-5-methoxy-1,2-benzoxazol-3-yl}-4-[1-(3-fluoroazetidin-1-yl)ethyl]-2,6-dimethoxybenzene-1-sulfonamide

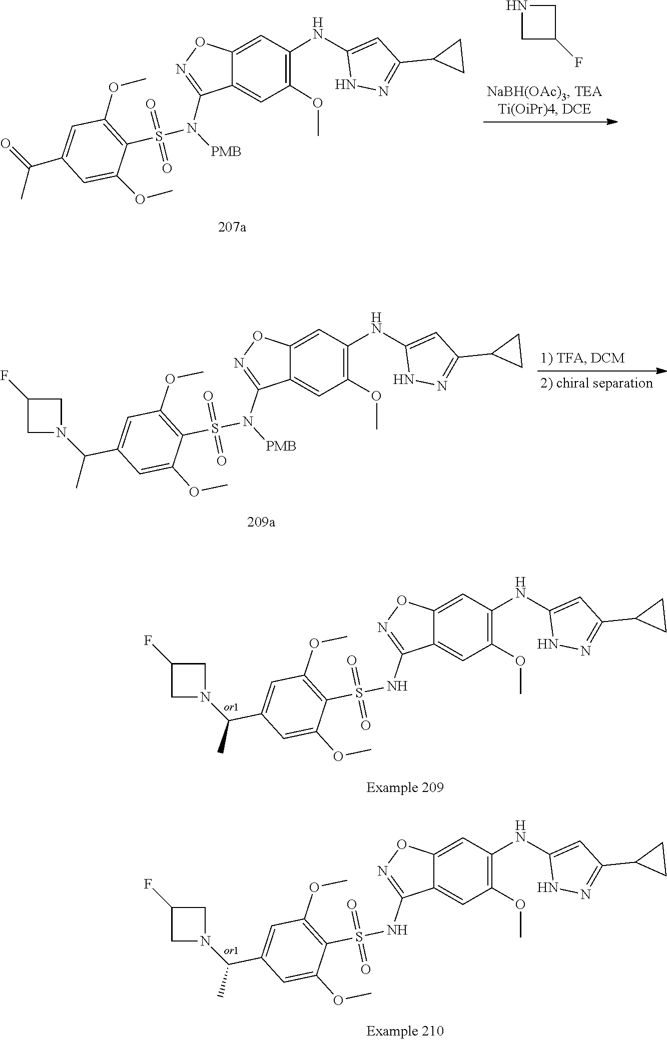

### Step 1: Synthesis of N-{6-[(5-cyclopropyl-1H-pyrazol-3-yl)amino]-5-methoxy-1,2-benzoxazol-3-yl}-4-[1-(3-fluoroazetidin-1-yl)ethyl]-2,6-dimethoxy-N-[(4-methoxyphenyl)methyl]benzene-1-sulfonamide (209a)

To a solution of 207a and 3-fluoroazetidine (107 mg, 0.961 mmol) in DCE (7.39 mL) was added triethylamine (598 mg, 5.91 mmol). The reaction mixture was treated with titanium (IV) isopropoxide (525 mg, 1.48 mmol) and was then stirred at 20° C. for 90 mins. Sodium triacetoxyborohydride (470 mg, 2.22 mmol) was then added portionwise and the reaction mixture was stirred at 10° C. for 16 hrs. The resulting mixture was quenched with a sat. aq. solution of NaHCO₃ and filtered. The filtrate was extracted three times with DCM and the organic phase was dried and concentrated under reduced pressure. The resulting crude 209a (520 mg, >99%) was used in the next step without further purification, LCMS m/z 707.2 (M+H)⁺

### Step 2: Synthesis of N-{6-[(5-cyclopropyl-1H-pyrazol-3-yl)amino]-5-methoxy-1,2-benzoxazol-3-yl}-4-[1-(3-fluoroazetidin-1-yl)ethyl]-2,6-dimethoxybenzene-1-sulfonamide (Examples 209 and 210)

To a solution of N-{6-[(5-cyclopropyl-1H-pyrazol-3-yl)amino]-5-methoxy-1,2-benzoxazol-3-yl}-4-[1-(3-fluoroazetidin-1-yl)ethyl]-2,6-dimethoxy-N-[(4-methoxyphenyl)methyl]benzene-1-sulfonamide 209a (520 mg, 0.736 mmol) in DCM (10 mL) was added TFA (5 mL) at 15° C. The reaction mixture was stirred at that temperature for 16 hrs and was then concentrated under reduced pressure. The resulting crude was diluted with DMF (2.5 mL) and purified by preparative HPLC (C18 150×40 mm column, Mobile phase A: H₂O+NH₃H₂O+NH₄HCO₃ Mobile phase B: ACN 22-42% over 9 min, Flow rate 60 mL/min) to afford a racemic mixture of 209 and 210 (140 mg, 32.4%) as a white solid, LCMS m/z 587.3 (M+H)⁺. The enantiomers were separated by chiral SFC (60% ethanol with 0.1% ammonium hydroxide in CO₂; 80 mL/min flow rate; Column: Daicel Chiralpak® IG (250×30 mm; 10 □m))

### N-{6-[(5-cyclopropyl-1H-pyrazol-3-yl)amino]-5-methoxy-1,2-benzoxazol-3-yl}-4-[1-(3-fluoroazetidin-1-yl)ethyl]-2,6-dimethoxybenzene-1-sulfonamide (Example 209)

The first eluting peak was lyophilized and gave 209 (29.6 mg, 21.1%) as a white solid. ¹H NMR (400 MHZ, CDCl₃) δ 7.81 (s, 1H), 7.46 (s, 1H), 6.98 (s, 1H), 6.57 (s, 2H), 5.70 (s, 1H), 5.10 (br. d, J=3.1 Hz, 1H), 3.99 (s, 3H), 3.92 (s, 6H), 3.7-3.6 (m, 1H), 3.5-3.4 (m, 1H), 3.27 (br. s, 1H), 3.2-3.1 (m, 1H), −3.1-2.9 (m, 1H), 1.9-1.8 (m, 1H), 1.19 (br. d, J=6.6 Hz, 3H), 1.0-1.0 (m, 2H), 0.8-0.7 (m, 2H), LCMS m/z 587.3 (M+H)⁺.

### N-{6-[(5-cyclopropyl-1H-pyrazol-3-yl)amino]-5-methoxy-1,2-benzoxazol-3-yl}-4-[1-(3-fluoroazetidin-1-yl)ethyl]-2,6-dimethoxybenzene-1-sulfonamide (Example 210)

The second eluting peak was lyophilized and gave 210 (49.5 mg, 35.4%) as a white solid. 1H NMR (400 MHZ, CDCl₃) δ 7.71 (s, 1H), 7.37 (s, 1H), 6.89 (s, 1H), 6.48 (s, 2H), 5.61 (s, 1H), 5.2-4.9 (m, 1H), 3.90 (s, 3H), 3.83 (s, 6H), 3.7-3.6 (m, 1H), 3.4-3.3 (m, 1H), 3.2-3.2 (m, 1H), 3.2-3.0 (m, 1H), 3.0-2.9 (m, 1H), 1.76 (s, 1H), 1.10 (d, J=6.4 Hz, 3H), 0.9-0.9 (m, 2H), 0.7-0.6 (m, 2H); LCMS m/z 587.3 (M+H)⁺.

### Example 211: N-{6-[(3-cyclopropyl-1H-pyrazol-5-yl)amino]-5-methoxy-1,2-benzoxazol-3-yl}-2,6-dimethoxy-4-(pyrimidin-2-yl)benzene-1-sulfonamide

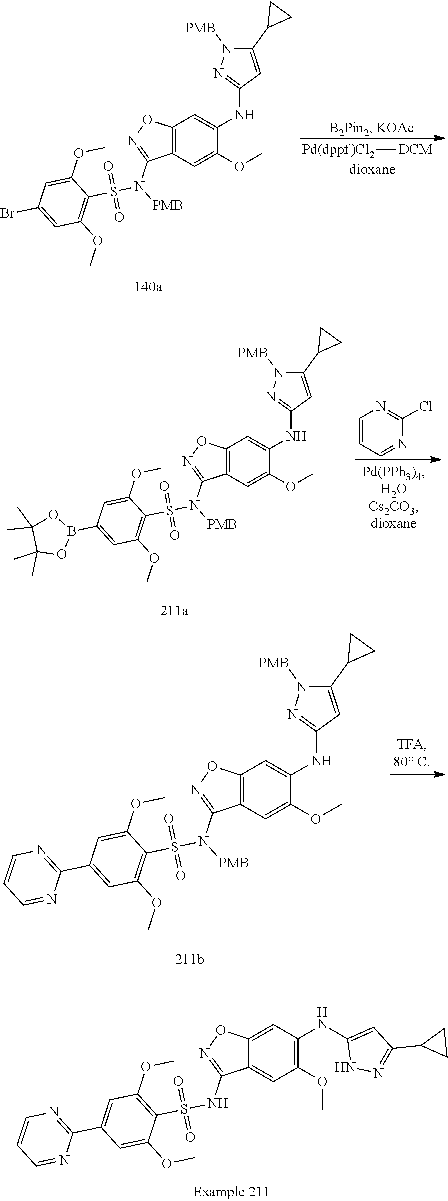

### Step 1: Synthesis of N-[6-({5-cyclopropyl-1-[(4-methoxyphenyl)methyl]-1H-pyrazol-3-yl}amino)-5-methoxy-1,2-benzoxazol-3-yl]-2,6-dimethoxy-N-[(4-methoxyphenyl)methyl]-4-(4,4,5,5-tetramethyl-1,3,2-dioxaborolan-2-yl)benzene-1-sulfonamide (211a)

To a solution of 4-Bromo-N-[6-({3-cyclopropyl-1-[(4-methoxyphenyl)methyl]-1H-pyrazol-5-yl}amino)-5-methoxy-1,2-benzoxazol-3-yl]-2,6-dimethoxy-N-[(4-methoxyphenyl)methyl]benzene-1-sulfonamide (140a) in dioxane (4.0 mL) was added bis (pinacolato)diboron (75.7 mg, 0.298 mmol), potassium acetate (23.4 mg, 0.239 mmol) and (1,1′-bis(diphenylphosphino) ferrocene) dichloropalladium-dichloromethane (1:1) (16.2 mg, 0.199 mmol). The mixture was stirred at 80° C. under nitrogen for 40 mins, then allowed to cool to rt. The mixture was filtered, and the filtrate was concentrated under reduced pressure to afford 211a as a black gum which was carried forward asis to the next step (276 mg, quant. yield). LCMS m/z 852.3 (M+H)⁺.

### Step 2: Synthesis of N-[6-({5-cyclopropyl-1-[(4-methoxyphenyl)methyl]-1H-pyrazol-3-yl}amino)-5-methoxy-1,2-benzoxazol-3-yl]-2,6-dimethoxy-N-[(4-methoxyphenyl)methyl]-4-(pyrimidin-2-yl)benzene-1-sulfonamide (211b)

To a solution of N-[6-({5-cyclopropyl-1-[(4-methoxyphenyl)methyl]-1H-pyrazol-3-yl}amino)-5-methoxy-1,2-benzoxazol-3-yl]-2,6-dimethoxy-N-[(4-methoxyphenyl)methyl]-4-(4,4,5,5-tetramethyl-1,3,2-dioxaborolan-2-yl)benzene-1-sulfonamide 211a (135 mg, 0.158 mmol) and 2-chloropyrimidine (18.2 mg, 0.158 mmol) in dioxane (2.4 mL) and water (0.6 mL) was added tetrakis (triphenylphosphine) palladium (9.16 mg, 0.00792 mmol) and cesium carbonate (155 mg, 0.475 mmol). The mixture was degassed and purged with nitrogen 3 times, then heated to 90° C. and stirred for 3.5 hrs. The resulting mixture was combined with a separate batch created using an identical procedure on a 20 mg scale, and the combined material was filtered and concentrated under reduced pressure. This afforded 211b as a black gum. Quantitative yield was assumed and the material was carried forward as is to the next step (320 mg). LCMS m/z 804.2 (M+H)⁺.

### Step 3: Synthesis of N-{6-[(5-cyclopropyl-1H-pyrazol-3-yl)amino]-5-methoxy-1,2-benzoxazol-3-yl}-2,6-dimethoxy-4-(pyrimidin-2-yl)benzene-1-sulfonamide (Example 211)

A solution of N-[6-({5-cyclopropyl-1-[(4-methoxyphenyl)methyl]-1H-pyrazol-3-yl}amino)-5-methoxy-1,2-benzoxazol-3-yl]-2,6-dimethoxy-N-[(4-methoxyphenyl)methyl]-4-(pyrimidin-2-yl)benzene-1-sulfonamide 211b (320 mg, 0.36 mmol) in TFA (8 mL, 0.05 M) was stirred at 80° C. for 16 hrs. The resulting mixture was concentrated under reduced pressure to afford a crude residue, which was re-diluted in DMF (5 mL) and purified via preparative HPLC (Phenomenex Gemini NX 150×30 mm column, 5 um particle size, Mobile phase A: water+ammonia hydroxide Mobile phase B: ACN 6-46% over 9 min, flow rate 60 mL/mins) to afford Example 211 as a yellow solid (13.5 mg, 6%). ¹H NMR (400 MHZ, METHANOL-d₄) δ 8.87 (d, J=4.89 Hz, 2H), 7.80 (s, 2H), 7.64 (br. s, 1H), 7.42 (t, J=4.89 Hz, 1H), 7.32 (s, 1H), 5.75 (s, 1H), 3.97-3.94 (m, 9H), 1.88 (s, 1H), 1.01-0.93 (m, 2H), 0.73 (dd, J=4.83, 2.02 Hz, 2H); LCMS m/z 564.3 (M+H)⁺.

### Example 212: N-{6-[(3-cyclopropyl-1H-pyrazol-5-yl)amino]-5-methoxy-1,2-benzoxazol-3-yl}-2,6-dimethoxy-4-(2-methyl-2H-1,2,3-triazol-4-yl)benzene-1-sulfonamide

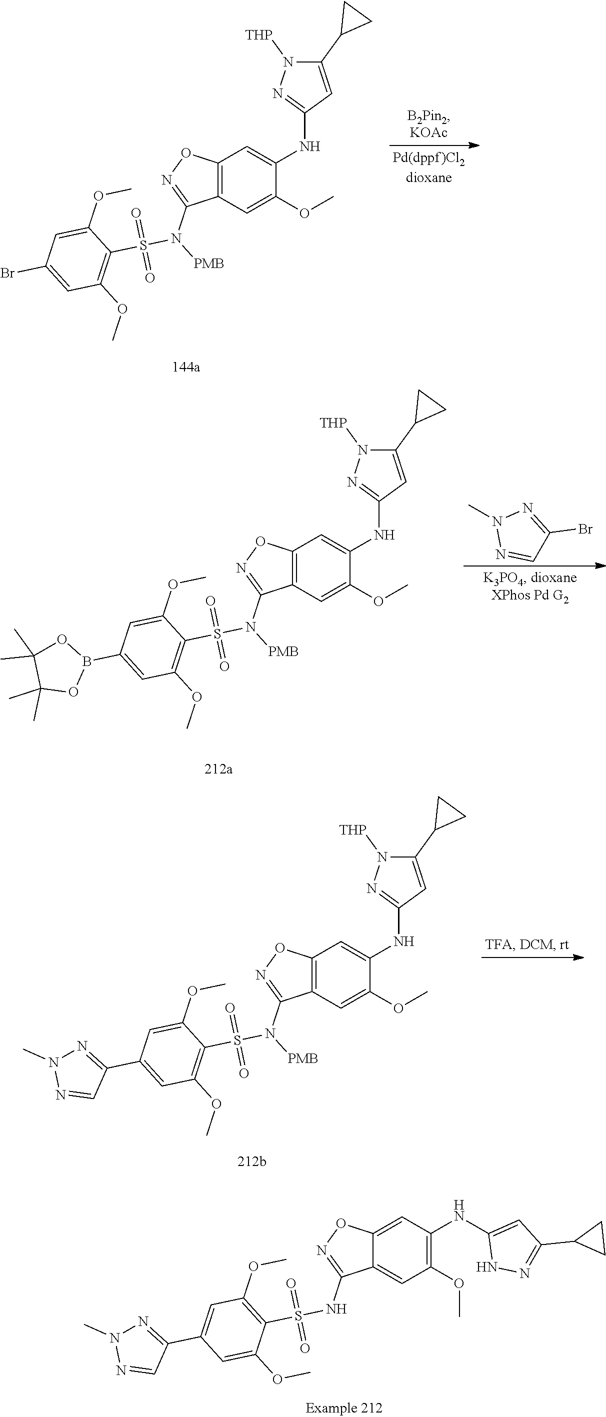

### Step 1: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(4,4,5,5-tetramethyl-1,3,2-dioxaborolan-2-yl)benzene-1-sulfonamide (212a)

To a solution of 4-bromo-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide (144a) (500 mg, 0.65 mmol) in dioxane (5 mL) was added bis (pinacolato)diboron (248 mg, 0.976 mmol), potassium acetate (76.6 mg, 0.781 mmol) and Pd(dppf)Cl₂ (47.6 mg, 0.650 mmol). The mixture was stirred at 80° C. under nitrogen for 3 hrs, then allowed to cool to 26° C. The mixture was filtered, and the filtrate was concentrated under reduced pressure to afford 212a as a black gum, which was used directly in the next step without further purification (860 mg, quant. yield). LCMS m/z 816.5 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(2-methyl-2H-1,2,3-triazol-4-yl)benzene-1-sulfonamide (212b)

To a solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(4,4,5,5-tetramethyl-1,3,2-dioxaborolan-2-yl)benzene-1-sulfonamide 212a (460 mg, 0.348 mmol) and 4-bromo-2-methyl-2H-1,2,3-triazole (40.0 mg, 0.25 mmol) in dioxane (1.23 mL) was added tripotassium phosphate (157 mg, 0.71 mmol). The suspension was degassed and purged 3 times with nitrogen. XPhos Pd G2 (9.71 mg, 0.0123 mmol) was added and the resulting mixture was degassed and purged twice with nitrogen. The suspension was stirred at 90° C. for 18 hrs under a nitrogen atmosphere. Water (5 mL) was added and the resulting mixture was stirred for 10 mins. The mixture was extracted with EtOAc (3×5 mL) and the combined organic layers were washed with brine (3×5 mL), dried over Na₂SO₄, filtered, and concentrated under reduced pressure. The crude residue was purified by flash column chromatography (ISCO, EtOAc/Pet. ether 0-100%) to afford 212b as a white gum (100 mg, 53%). LCMS m/z 771.2 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-(2-methyl-2H-1,2,3-triazol-4-yl)benzene-1-sulfonamide (Example 212)

A solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(2-methyl-2H-1,2,3-triazol-4-yl)benzene-1-sulfonamide 212b (100 mg, 0.130 mmol) in TFA (2 mL) and DCM (1 mL) was stirred at 25° C. for 4 hrs. The resulting mixture was diluted with DMF (2.5 mL) and purified by preparative HPLC (C18 150×40 mm column, Mobile phase A: water+formic acid Mobile phase B: ACN 23-63% over 9 mins, flow rate 60 mL/mins) to afford Example 212 as a white solid (46.0 mg, 63%). ¹H NMR (400 MHZ, DMSO-d₆) d 11.90 (br. s, 1H), 11.05 (br. s, 1H), 8.41 (s, 1H), 8.16-8.08 (m, 1H), 8.05 (s, 1H), 7.41 (s, 1H), 7.15 (s, 2H), 5.78 (s, 1H), 4.21 (s, 3H), 3.88 (s, 3H), 3.85 (s, 6H), 1.89-1.82 (m, 1H), 0.95-0.88 (m, 2H), 0.69-0.63 (m, 2H); LCMS m/z 567.3 (M+H)⁺.

The Examples in Table 23 were made in a similar fashion as Example 212 using commercially available hetero-aryl chlorides or bromides for the Suzuki reaction (see step 2).

TABLE 23

| Example | | |
|---|---|---|
| Number | Structure/IUPAC Name | Analytical Data |
| 213 | 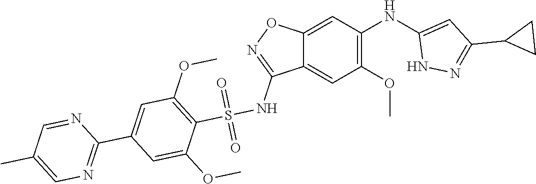 [SMILES: COc1cc2c(NS(=O)(=O)c3c(OC)cc(-c4ncc(C)cn4)cc3OC)noc2cc1Nc1cc(C2CC2)n[nH]1] | LCMS m/z 578.3 (M + H)⁺; ¹H NMR (400 MHz, DMSO-d₆) d 11.91-11.86 (m, 1H), 11.11 (s, 1H), 8.80 (s, 2H), 8.15-8.04 (m, 2H), 7.68 (s, 2H), 7.44-7.40 (m, 1H), 5.80-5.74 (m, 1H), 3.90-3.85 (m, 9H), 2.33 (s, 3H), 1.88-1.80 (m, 1H), 0.93-0.87 (m, 2H), 0.68-0.62 (m, 2H). |
| | N-{6-[(3-cyclopropyl-1H-pyrazol-5- | |
| | yl)amino]-5-methoxy-1,2-benzoxazol-3- | |
| | yl}-2,6-dimethoxy-4-(5-methylpyrimidin- | |
| | 2-yl)benzene-1-sulfonamide | |
| 214 | 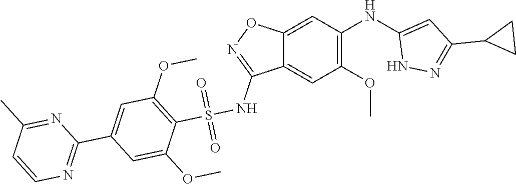 [SMILES: COc1cc2c(NS(=O)(=O)c3c(OC)cc(-c4nccc(C)n4)cc3OC)noc2cc1Nc1cc(C2CC2)n[nH]1] | LCMS m/z 578.3 (M + H)⁺; ¹H NMR (400 MHz, DMSO-d₆) d 11.89 (s, 1H), 11.11 (s, 1H), 8.79 (d, J = 5.06 Hz, 1H), 8.16-8.05 (m, 2H), 7.70 (s, 2H), 7.43-7.40 (m, 2H), 5.77 (s, 1H), 3.90-3.86 (m, 9H), 2.55 (s, 3H), 1.84 (s, 1H), 0.90 (br. dd, J = 8.36, 2.20 Hz, 2H), 0.69- 0.61 (m, 2H). |
| | N-{6-[(3-cyclopropyl-1H-pyrazol-5- | |
| | yl)amino]-5-methoxy-1,2-benzoxazol-3- | |
| | yl}-2,6-dimethoxy-4-(4-methylpyrimidin- | |
| | 2-yl)benzene-1-sulfonamide | |
| 215 | 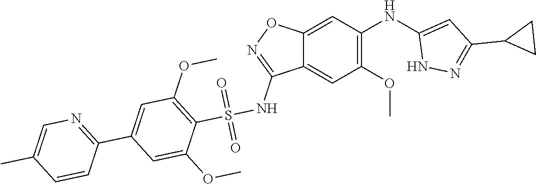 [SMILES: COc1cc2c(NS(=O)(=O)c3c(OC)cc(-c4ccc(C)cn4)cc3OC)noc2cc1Nc1cc(C2CC2)n[nH]1] | LCMS m/z 577.3 (M + H)⁺; ¹H NMR (400 MHz, DMSO-d₆) d 11.89 (br. s, 1H), 11.12-10.94 (m, 1H), 8.14-8.10 (m, 1H), 8.06 (s, 1H), 7.91-7.87 (m, 1H), 7.82-7.77 (m, 1H), 7.42 (s, 1H), 7.36 (s, 2H), 7.29 (d, J = 7.5 Hz, 1H), 5.78-5.76 (m, 1H), 3.90-3.85 (m, 9H), 2.54 (s, 3H), 1.88-1.81 (m, 1H), 0.94-0.87 (m, 2H), 0.68-0.62 (m, 2H). |
| | N-{6-[(3-cyclopropyl-1H-pyrazol-5- | |
| | yl)amino]-5-methoxy-1,2-benzoxazol-3- | |
| | yl}-2,6-dimethoxy-4-(5-methylpyridin-2- | |
| | yl)benzene-1-sulfonamide | |
| 216 | 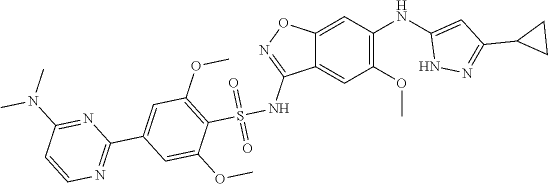 [SMILES: COc1cc2c(NS(=O)(=O)c3c(OC)cc(-c4nccc(N(C)C)n4)cc3OC)noc2cc1Nc1cc(C2CC2)n[nH]1] | LCMS m/z 607.3 (M + H)⁺; ¹H NMR (400 MHz, DMSO-d₆) d 11.88 (br. s, 1H), 11.02 (br. dd, J = 5.81, 3.48 Hz, 1H), 8.29 (d, J = 6.11 Hz, 1H), 8.10 (br. s, 1H), 8.04-7.94 (m, 1H), 7.66 (s, 2H), 7.40 (s, 1H), 6.67 (d, J = 6.24 Hz, 1H), 5.77 (s, 1H), 3.87 (s, 3H), 3.85 (s, 6H), 3.19-3.05 (m, 6H), 1.91- .79 (m, 1H), 0.95-0.85 (m, 2H), 0.72-0.58 (m, 2H). |
| | N-{6-[(3-cyclopropyl-1H-pyrazol-5- | |
| | yl)amino]-5-methoxy-1,2-benzoxazol-3- | |
| | yl}-4-[4-(dimethylamino)pyrimidin-2-yl]- | |
| | 2,6-dimethoxybenzene-1-sulfonamide | |
| 217 | 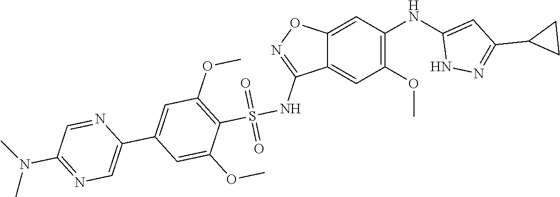 [SMILES: COc1cc2c(NS(=O)(=O)c3c(OC)cc(-c4cnc(N(C)C)cn4)cc3OC)noc2cc1Nc1cc(C2CC2)n[nH]1] | LCMS m/z 607.3 (M + H)⁺; ¹H NMR (400 MHz, DMSO-d₆) d 11.93-11.85 (m, 1H), 11.03-10.89 (m, 1H), 8.84-8.80 (m, 1H), 8.23 (d, J = 1.1 Hz, 1H), 8.15-8.09 (m, 1H), 8.08-8.01 (m, 1H), 7.45-7.40 (m, 1H), 7.28 (s, 2H), 5.77 (s, 1H), 3.87 (s, 3H), 3.86-3.83 (m, 6H), 3.15- 3.11 (m, 6H), 1.88-1.81 (m, 1H), 0.94-0.87 (m, 2H), 0.68-0.62 (m, 2H). |
| | N-{6-[(3-cyclopropyl-1H-pyrazol-5- | |
| | yl)amino]-5-methoxy-1,2-benzoxazol-3- | |
| | yl}-4-[5-(dimethylamino)pyrazin-2-yl]- | |
| | 2,6-dimethoxybenzene-1-sulfonamide | |
| 218 | 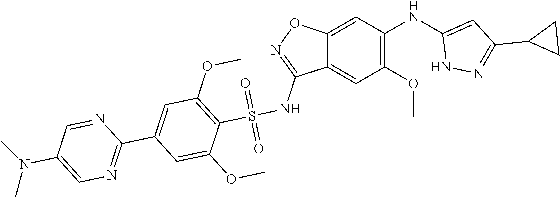 [SMILES: COc1cc2c(NS(=O)(=O)c3c(OC)cc(-c4ncc(N(C)C)cn4)cc3OC)noc2cc1Nc1cc(C2CC2)n[nH]1] | LCMS m/z 607.3 (M + H)⁺; ¹H NMR (400 MHz, DMSO-d₆) d 11.92-11.85 (m, 1H), 11.05-10.97 (m, 1H), 8.40 (s, 2H), 8.05 (s, 2H), 7.56 (s, 2H), 7.45-7.39 (m, 1H), 5.80-5.75 (m, 1H), 3.90-3.87 (m, 3H), 3.86-3.82 (m, 6H), 3.03 (s, 6H), 1.89-1.81 (m, 1H), 0.95-0.88 (m, 2H), 0.68-0.62 (m, 2H). |
| | N-{6-[(3-cyclopropyl-1H-pyrazol-5- | |
| | yl)amino]-5-methoxy-1,2-benzoxazol-3- | |
| | yl}-4-[5-(dimethylamino)pyrimidin-2-yl]- | |
| | 2,6-dimethoxybenzene-1-sulfonamide | |
| 219 | 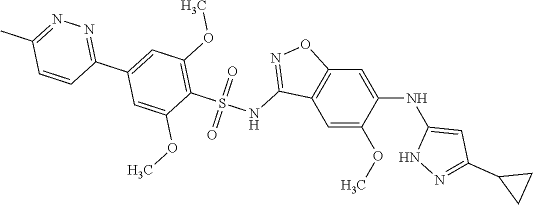 [SMILES: COc1cc2c(NS(=O)(=O)c3c(OC)cc(-c4ccc(C)nn4)cc3OC)noc2cc1Nc1cc(C2CC2)n[nH]1] | ¹H NMR (400 MHz, DMSO-d₆) δ 11.93-11.85 (m, 1H), 11.19-11.03 (m, 1H), 8.29 (d, J = 8.9 Hz, 1H), 8.17-8.04 (m, 2H), 7.72 (d, J = 8.9 Hz, 1H), 7.47-7.41 (m, 3H), 5.78 (s, 1H), 3.89 (d, J = 2.5 Hz, 9H), 2.68 (s, 3H), 1.89-1.82 (m, 1H), 0.94- 0.89 (m, 2H), 0.68-0.63 (m, 2H); LCMS m/z 578.3 (M + H)⁺; rt = 0.778 |
| | N-{6-[(3-cyclopropyl-1H-pyrazol-5- | |
| | yl)amino]-5-methoxy-1,2-benzoxazol-3- | |
| | yl}-2,6-dimethoxy-4-(6-methylpyridazin- | |
| | 3-yl)benzene-1-sulfonamide | |
| 220 | 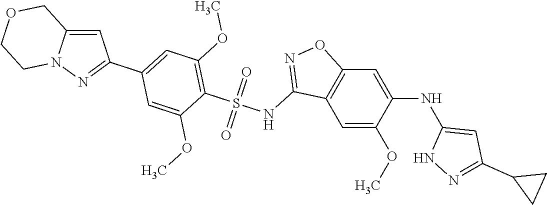 [SMILES: COc1cc2c(NS(=O)(=O)c3c(OC)cc(-c4cc5n(n4)CCOC5)cc3OC)noc2cc1Nc1cc(C2CC2)n[nH]1] | ¹H NMR (400 MHz, DMSO-d₆) δ 12.02-11.79 (m, 1H) 11.28-10.79 (m, 1H) 8.13-8.00 (m, 2H) 7.40 (s, 1H) 7.07 (s, 2H) 6.70 (s, 1H) 5.77 (s, 1H) 4.82 (s, 2H) 4.16 (br. s, 2H) 4.08 (br. d, J = 5.14 Hz, 2H) 3.87 (s, 3H) 3.81 (s, 6H) 1.90-1.80 (m, 1H) 0.96-0.87 (m, 2H) 0.70-0.60 (m, 2H); LCMS m/z 608.3 (M + H)⁺; rt = 0.830 |
| | N-{6-[(3-cyclopropyl-1H-pyrazol-5- | |
| | yl)amino]-5-methoxy-1,2-benzoxazol-3- | |
| | yl}-4-(6,7-dihydro-4H-pyrazolo[5,1- | |
| | c][1,4]oxazin-2-yl)-2,6- | |
| | dimethoxybenzene-1-sulfonamide | |
| 221 | 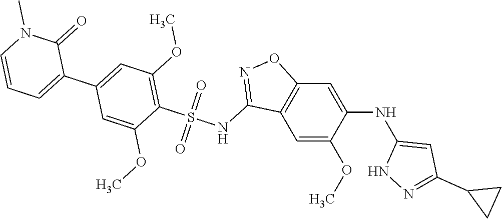 [SMILES: COc1cc2c(NS(=O)(=O)c3c(OC)cc(-c4cccn(C)c4=O)cc3OC)noc2cc1Nc1cc(C2CC2)n[nH]1] | ¹H NMR (400 MHz, DMSO-d₆) δ 11.90 (br s, 1H), 11.12-10.86 (m, 1H), 8.20-7.99 (m, 2H), 7.82 (d, J = 7.0 Hz, 2H), 7.40 (s, 1H), 7.12 (s, 2H), 6.34 (t, J = 7.0 Hz, 1H), 5.78 (s, 1H), 3.87 (s, 3H), 3.78 (s, 6H), 3.50 (s, 3H), 1.94-1.74 (m, 1H), 1.01-0.79 (m, 2H), 0.75-0.59 (m, 2H); LCMS m/z 593.3 (M + H)⁺; rt = 0.830 |
| | N-{6-[(3-cyclopropyl-1H-pyrazol-5- | |
| | yl)amino]-5-methoxy-1,2-benzoxazol-3- | |
| | yl}-2,6-dimethoxy-4-(1-methyl-2-oxo- | |
| | 1,2-dihydropyridin-3-yl)benzene-1- | |
| | sulfonamide | |

### Example 222: N-{6-[(3-cyclopropyl-1H-pyrazol-5-yl)amino]-5-methoxy-1,2-benzoxazol-3-yl}-4-{4-[(dimethylamino) methyl]pyrimidin-2-yl}-2,6-dimethoxybenzene-1-sulfonamide

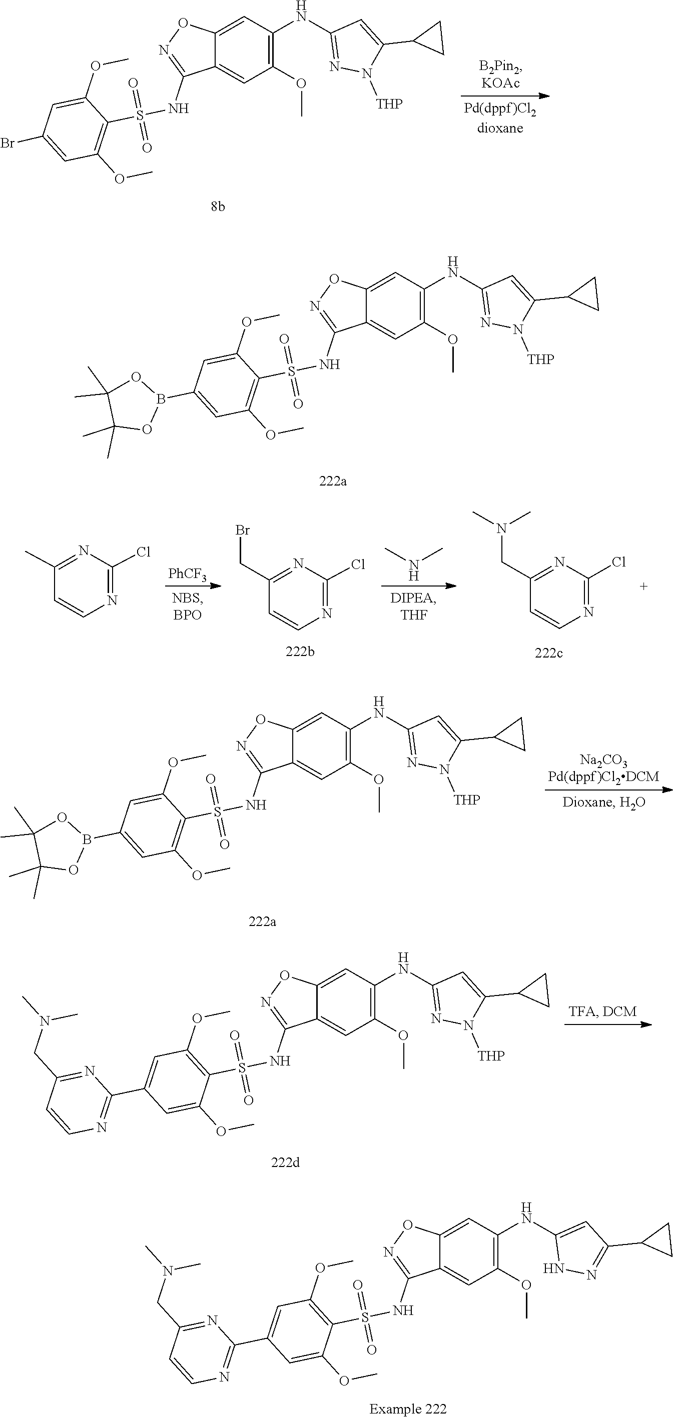

### Step 1: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-(4,4,5,5-tetramethyl-1,3,2-dioxaborolan-2-yl)benzene-1-sulfonamide (222a)

To a solution of 8b (800 mg, 1.23 mmol) in dioxane (10 mL) was added bis (pinacolato)diboron (470 mg, 1.85 mmol), potassium acetate (145 mg, 1.48 mmol) and (1,1′-bis(diphenylphosphino) ferrocene) dichloropalladium (90.3 mg, 0.123 mmol). The mixture was stirred at 80° C. under nitrogen for 3 hrs. The mixture was filtered and concentrated to give crude 222a (1400 mg, 100%) as black gum, which was used directly without purification. LCMS m/z 614.1 (M+H)⁺ (mass of the boronic acid)

### Step 2: Synthesis of 4-(bromomethyl)-2-chloropyrimidine (222b)

To a solution of 2-chloro-4-methylpyrimidine (500 mg, 3.89 mmol) in (trifluoromethyl)benzene (15.0 mL) was added N-bromosuccinimide (1.04 g, 5.83 mmol) and benzoyl peroxide (47.1 mg, 0.194 mmol). The mixture was stirred at 80° C. for 16 hrs. LCMS showed that the reaction had stalled, so additional benzoyl peroxide (94.2 mg, 0.389 mmol) was added and the mixture was stirred at 80° C. for 2.5 hrs. This mixture was combined with a separate batch made using an identical procedure on a 500 mg scale and the combined material was filtered and concentrated under reduced pressure. The crude residue was purified by column chromatography (40 g, silica gel, 9-15% EtOAc/Pet. ether) to afford 222b (400 mg, 50%) as a yellow oil. LCMS m/z 209.0 (M+H)⁺.

### Step 3: Synthesis of 1-(2-chloropyrimidin-4-yl)-N,N-dimethylmethanamine (222c)

To a solution of 4-(bromomethyl)-2-chloropyrimidine 222b (300 mg, 1.45 mmol) in THF (3 mL) was added DIPEA (934 mg, 7.23 mmol) and dimethylamine (236 mg, 2.89 mmol). The mixture was stirred at rt for 16 hrs, then concentrated under reduced pressure. The crude residue was purified by column chromatography (20 g silica gel, 0-3% DCM/MeOH) to afford 222c (125 mg, 50%) as a yellow oil. ¹H NMR (400 MHZ, DMSO-d₆) δ 8.72 (d, J=5.01 Hz, 1H) 7.57 (d, J=5.14 Hz, 1H) 3.54 (s, 2H) 2.22 (s, 6H); LCMS m/z 172.1 (M+H)⁺.

### Step 4: Synthesis of N-(6-{[5-cyclopropyl-1-(oxan-2-yl)-1H-pyrazol-3-yl]amino}-5-methoxy-1,2-benzoxazol-3-yl)-4-{4-[(dimethylamino) methyl]pyrimidin-2-yl}-2,6-dimethoxybenzene-1-sulfonamide (222d)

To a solution of 222a (152 mg, 0.175 mmol) in dioxane (1.6 mL) and water (0.4 mL) was added 222c (25.0 mg, 0.15 mmol), sodium carbonate (69.5 mg, 0.655 mmol) and (1,1′-bis(diphenylphosphino) ferrocene) dichloropalladium-dichloromethane (1:1) (11.9 mg, 0.0146 mmol). The mixture was degassed and purged with nitrogen twice, then heated to 100° C. in a microwave for 2 hrs. The resulting mixture was combined with a separate batch created using an identical procedure on a 60 mg scale, and the combined material was filtered and concentrated under reduced pressure to afford 222d as a black oil, which was used in the next step without further purification (300 mg, quant. yield). LCMS m/z 705.4 (M+H)⁺.

### Step 5: Synthesis of N-{6-[(3-cyclopropyl-1H-pyrazol-5-yl)amino]-5-methoxy-1,2-benzoxazol-3-yl}-4-{4-[(dimethylamino) methyl]pyrimidin-2-yl}-2,6-dimethoxybenzene-1-sulfonamide (Example 222)

A solution of N-(6-{[5-cyclopropyl-1-(oxan-2-yl)-1H-pyrazol-3-yl]amino}-5-methoxy-1,2-benzoxazol-3-yl)-4-{4-[(dimethylamino) methyl]pyrimidin-2-yl}-2,6-dimethoxybenzene-1-sulfonamide 222d (300 mg, 0.426 mmol) in DCM (3.0 mL) and TFA (5.0 mL) was stirred at 15° C. for 3 hrs. The reaction was concentrated under reduced pressure and the resulting crude residue was diluted with DMF (5.5 mL) and purified by preparative HPLC (Phenomenex Gemini NX 150×30 mm column, Mobile phase A: Water+0.1% ammonium hydroxide Mobile phase B: ACN 2-42% over 9 mins, flow rate 60 mL/mins) to afford Example 222 (7.27 mg, 3%) as a white solid. ¹H NMR (400 MHZ, DMSO-d₆) d 11.90 (br. s, 1H), 11.24-10.09 (m, 1H), 8.90 (d, J=5.13 Hz, 1H), 8.13 (s, 1H), 8.06 (s, 1H), 7.70 (s, 2H), 7.54 (d, J=5.13 Hz, 1H), 7.42 (s, 1H), 5.78 (s, 1H), 3.92-3.85 (m, 9H), 3.66 (s, 2H), 2.26 (s, 6H), 1.91-1.80 (m, 1H), 0.96-0.86 (m, 2H), 0.69-0.59 (m, 2H); LCMS m/z 621.3 (M+H)⁺.

### Example 223: N-{6-[(3-cyclopropyl-1H-pyrazol-5-yl)amino]-5-methoxy-1,2-benzoxazol-3-yl}-4-{5-[(dimethylamino) methyl]pyrimidin-2-yl}-2,6-dimethoxybenzene-1-sulfonamide

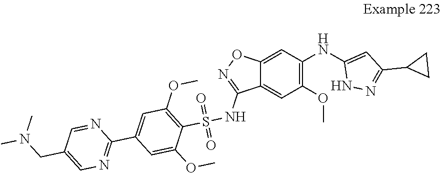 [SMILES: COc1cc2c(NS(=O)(=O)c3c(OC)cc(-c4ncc(CN(C)C)cn4)cc3OC)noc2cc1Nc1cc(C2CC2)n[nH]1]

Example 223 was made in a similar manner as Example 222 using 2-chloro-5-methylpyrimidine in place of 2-chloro-4-methylpyrimidine in step 2. ¹H NMR (400 MHZ, DMSO-d₆) δ 12.03-11.79 (m, 1H), 11.49-10.79 (m, 1H), 8.84 (s, 2H), 8.12-8.07 (m, 1H), 8.07-8.04 (m, 1H), 7.70 (s, 2H), 7.42-7.39 (m, 1H), 5.77 (s, 1H), 3.89-3.85 (m, 9H), 3.49 (s, 2H), 2.17 (s, 6H), 1.88-1.81 (m, 1H), 0.93-0.87 (m, 2H), 0.67-0.62 (m, 2H); LCMS m/z 621.3 (M+H)⁺.

### Example 224:4-(5-cyanopyrimidin-2-yl)-N-{6-[(3-cyclopropyl-1H-pyrazol-5-yl)amino]-5-methoxy-1,2-benzoxazol-3-yl}-2,6-dimethoxybenzene-1-sulfonamide

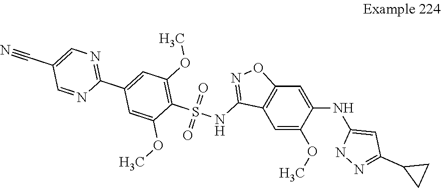

Example 224 was prepared analogous to Example 222 with 2-chloropyrimidine-5-carbonitrile being used as the coupling partner and heated to 80° C. during the coupling reaction (step 4) to provide Example 224 (12 mg, 16% yield) as a yellow solid. ¹H NMR (400 MHZ, DMSO-d₆) δ 11.90 (br. s, 1H) 11.21 (br. d, J=1.50 Hz, 1H) 9.43 (s, 2H) 8.02-8.17 (m, 2H) 7.73 (s, 2H) 7.40 (s, 1H) 5.77 (s, 1H) 3.88 (s, 9H) 1.79-1.90 (m, 1H) 0.85-0.96 (m, 2H) 0.60-0.70 (m, 2H); LCMS m/z 589.2 (M+H)⁺; rt=0.830.

### Example 225: N-{6-[(3-cyclopropyl-1H-pyrazol-5-yl)amino]-5-methoxy-1,2-benzoxazol-3-yl}-2.6-dimethoxy-4-(7-methyl-5,6,7,8-tetrahydropyrido[3,4-d]pyrimidin-2-yl)benzene-1-sulfonamide

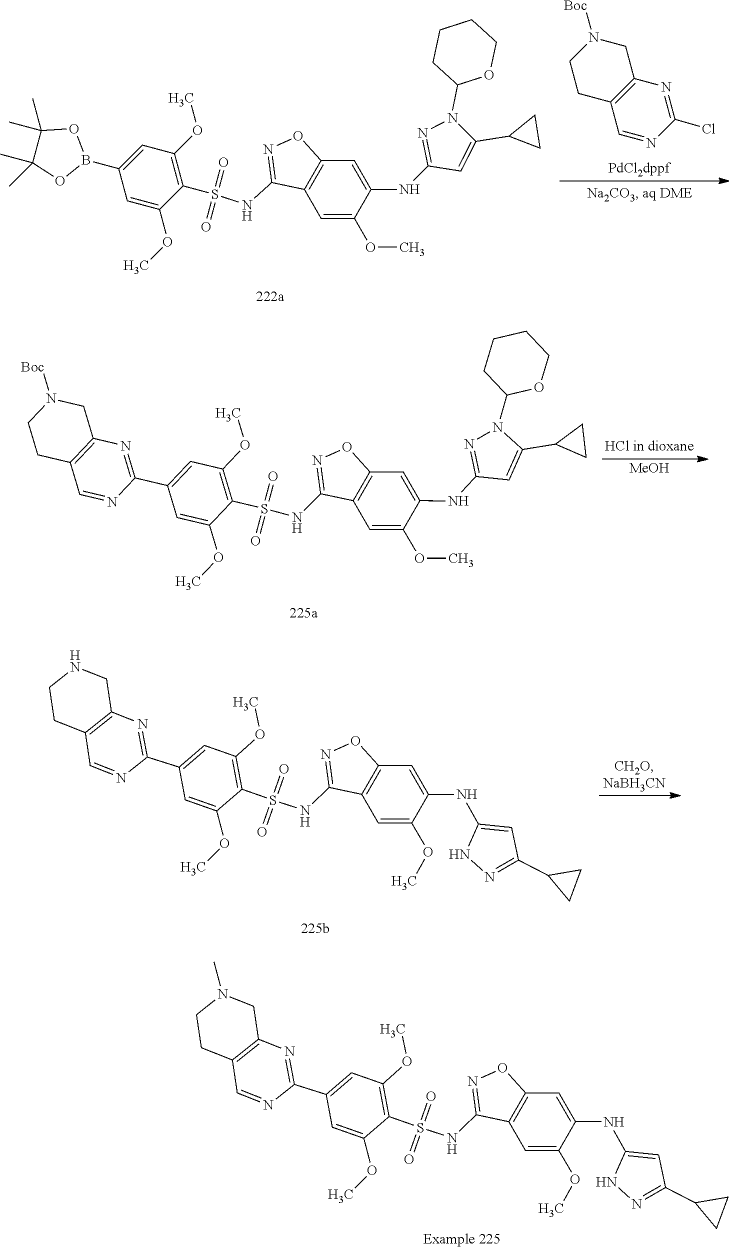 [SMILES: COc1cc2c(NS(=O)(=O)c3c(OC)cc(B4OC(C)(C)C(C)(C)O4)cc3OC)noc2cc1Nc1cc(C2CC2)n(C2CCCCO2)n1 ; "MeOH" ; CC(C)(C)OC(=O)N1CCc2cnc(Cl)nc2C1 ; COc1cc2c(NS(=O)(=O)c3c(OC)cc(-c4ncc5c(n4)CN(C(=O)OC(C)(C)C)CC5)cc3OC)noc2cc1Nc1cc(C2CC2)n(C2CCCCO2)n1 ; COc1cc2c(NS(=O)(=O)c3c(OC)cc(-c4ncc5c(n4)CNCC5)cc3OC)noc2cc1Nc1cc(C2CC2)n[nH]1 ; COc1cc2c(NS(=O)(=O)c3c(OC)cc(-c4ncc5c(n4)CN(C)CC5)cc3OC)noc2cc1Nc1cc(C2CC2)n[nH]1]

### Step 1: Synthesis of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}-5,8-dihydropyrido[3,4-d]pyrimidine-7 (6H)-carboxylate (225a)

To a solution of N-(6-{[5-cyclopropyl-1-(oxan-2-yl)-1H-pyrazol-3-yl]amino}-5-methoxy-1,2-benzoxazol-3-yl)-2,6-dimethoxy-4-(4,4,5,5-tetramethyl-1,3,2-dioxaborolan-2-yl)benzene-1-sulfonamide (222a) (600 mg, 0.69 mmol) in 1,2 dimethoxy ethane (7.5 mL) and water (2.5 mL) were added tert-butyl 2-chloro-5,8-dihydropyrido[3,4-d]pyrimidine-7 (6H)-carboxylate (170 mg, 0.630 mmol), 1,1′-Bis(diphenylphosphino)ferrocene dichloropalladium (II) (46.1 mg, 0.063 mmol), and sodium carbonate (200 mg, 1.89 mmol). The reaction mixture was degassed and purged with nitrogen, and the solution was heated at 60° C. for 5 hrs. The reaction mixture was concentrated and purified by column chromatography (SiO₂, Petroleum ether/Ethyl acetate=100/1 to 1/5) to give 225a (300 mg, 59% yield) as a yellow solid. LCMS m/z 803.4 (M+H)⁺, rt=1.105 min.

### Step 2: Synthesis of N-{6-[(3-cyclopropyl-1H-pyrazol-5-yl)amino]-5-methoxy-1,2-benzoxazol-3-yl}-2,6-dimethoxy-4-(5,6,7,8-tetrahydropyrido[3,4-d]pyrimidin-2-yl)benzene-1-sulfonamide (225b)

To a solution of tert-butyl 2-{4-[(6-{[5-cyclopropyl-1-(oxan-2-yl)-1H-pyrazol-3-yl]amino}-5-methoxy-1,2-benzoxazol-3-yl)sulfamoyl]-3,5-dimethoxyphenyl}-5,8-dihydropyrido[3,4-d]pyrimidine-7 (6H)-carboxylate (225a) (350 mg, 0.436 mmol) in anhydrous MeOH (1 mL) was added 4M HCl in dioxane (3 mL). The reaction mixture was stirred at 15° C. for 16 hrs, then 45° C. for 3 hrs. The reaction mixture was concentrated to give a crude mixture of 225b (320 mg, >95% yield) which was a yellow solid and was used directly in the next step. LCMS m/z 619.3 (M+H)⁺, rt=0.798 min.

### Step 3: Synthesis of N-{6-[(3-cyclopropyl-1H-pyrazol-5-yl)amino]-5-methoxy-1,2-benzoxazol-3-yl}-2,6-dimethoxy-4-(7-methyl-5,6,7,8-tetrahydropyrido[3,4-d]pyrimidin-2-yl)benzene-1-sulfonamide (Example 225)

To a suspension of N-{6-[(3-cyclopropyl-1H-pyrazol-5-yl)amino]-5-methoxy-1,2-benzoxazol-3-yl}-2,6-dimethoxy-4-(5,6,7,8-tetrahydropyrido[3,4-d]pyrimidin-2-yl)benzene-1-sulfonamide (225b) (100 mg, 0.153 mmol) in MeOH (2 mL) was added 37 wt % (aq.) formaldehyde solution (124 mg, 1.53 mmol) at 15° C. The reaction mixture was stirred at 15° C. for 20 min. Sodium cyanoborohydride (48 mg, 0.763 mmol) was added to the reaction mixture. The reaction mixture was stirred at 15° C. for 1 hr then filtered through celite and concentrated. The crude material was purified using preparative HPLC (C18 150×40 mm, 18-38% ACN: H₂O (modified with ammonium formate) over 9 min, flow rate 60 ml/mins). The product fractions were combined and lyophilized to give Example 225 (50 mg) with remaining impurities. This material was then purified using preparative SFC (Daicel Chiralpak IC (250×30 mm, 10 μm, eluting with CO₂/MeOH at a flow rate of 100 mL/mins) to afford Example 225 (15.5 mg, 16% yield) as a white solid. ¹H NMR (400 MHZ, METHANOL-d₄) δ 8.69-8.56 (m, 1H), 7.81-7.71 (m, 2H), 7.67-7.55 (m, 1H), 7.39-7.26 (m, 1H), 5.81-5.69 (m, 1H), 3.98-3.94 (m, 9H), 3.76-3.69 (m, 2H), 3.04-2.93 (m, 2H), 2.90-2.77 (m, 2H), 2.60-2.47 (m, 3H), 1.95-1.82 (m, 1H), 1.05-0.93 (m, 2H), 0.80-0.70 (m, 2H); LCMS m/z 633.3 (M+H)⁺; rt=0.849.

### Example 226: N-{6-[(3-cyclopropyl-1H-pyrazol-5-yl)amino]-5-methoxy-1,2-benzoxazol-3-yl}-2,6-dimethoxy-4-[5-(4-methylpiperazin-1-yl)pyrimidin-2-yl]benzene-1-sulfonamide

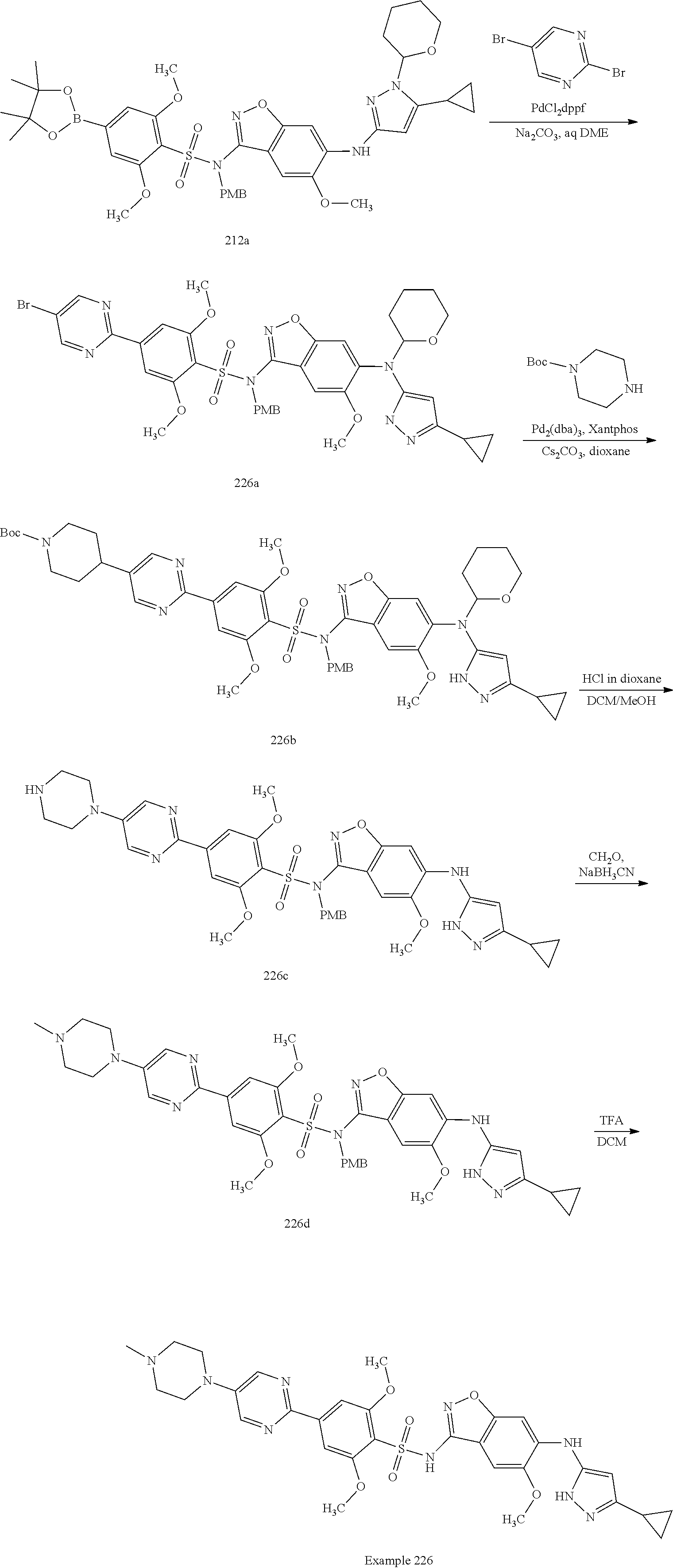

### Step 1: Synthesis of 4-(5-bromopyrimidin-2-yl)-N-{6-[(3-cyclopropyl-1H-pyrazol-5-yl) (oxan-2-yl)amino]-5-methoxy-1,2-benzoxazol-3-yl}-2,6-dimethoxy-N-[(4-methoxyphenyl)methyl]benzene-1-sulfonamide (226a)

To a solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(4,4,5,5-tetramethyl-1,3,2-dioxaborolan-2-yl)benzene-1-sulfonamide (212a) (1000 mg, 0.83 mmol) in 1,2 dimethoxy ethane (4.5 mL) and water (1.5 mL) were added were added 2,5-dibromopyrimidine (238 mg, 1.0 mmol), sodium carbonate (265 mg, 2.5 mmol), and 1,1′-Bis(diphenylphosphino) ferrocene dichloropalladium (II) (61.0 mg, 0.0834 mmol). The reaction mixture was degassed and purged with nitrogen, and the solution was heated to 60° C. The reaction mixture was stirred for 5 hrs. The reaction was diluted with water (10 mL) and washed with ethyl acetate (10 mL×3). The organic layers were combined, washed with brine (10 mL×2), dried (sodium sulfate), filtered, and concentrated to give a crude yellow solid. The crude residue was purified using column chromatography (SiO₂, Petroleum ether/Ethyl acetate=100/1 to 1/100) to give 226a (380 mg, 54% yield) as a yellow solid. LCMS m/z 846.3 (M+H), rt=0.97 min.

### Step 2: Synthesis of tert-butyl 4-{2-[4-({6-[(3-cyclopropyl-1H-pyrazol-5-yl) (oxan-2-yl)amino]-5-methoxy-1,2-benzoxazol-3-yl}[(4-methoxyphenyl)methyl]sulfamoyl)-3,5-dimethoxyphenyl]pyrimidin-5-yl}piperazine-1-carboxylate (226b)

To a yellow suspension of 4-(5-bromopyrimidin-2-yl)-N-{6-[(3-cyclopropyl-1H-pyrazol-5-yl) (oxan-2-yl)amino]-5-methoxy-1,2-benzoxazol-3-yl}-2,6-dimethoxy-N-[(4-methoxyphenyl)methyl]benzene-1-sulfonamide (226a) (380 mg, 0.449 mmol), tert-butyl piperazine-1-carboxylate (167 mg, 0.898 mmol), and cesium carbonate (439 mg, 1.35 mmol) in dioxane (4.49 mL) was bubbled nitrogen for 1 min. Tris (dibenzylideneacetone)dipalladium (0) (82.2 mg, 0.0898 mmol) and Xantphos (156 mg, 0.269 mmol) were added to the reaction mixture. The reaction mixture atmosphere was evacuated and backfilled with nitrogen. The reaction mixture was stirred at 100° C. for 18 hrs. The reaction mixture was cooled to ambient temperature, diluted with ethyl acetate (5 mL) and filtered. The filtrate was washed with brine (5 mL×2), dried over sodium sulfate, filtered, and concentrated to furnish a crude material. The crude product was purified by column chromatography (SiO₂, dichloromethane/methanol=100/1 to 20/1) to give 226b (300 mg, 70% yield) as a yellow solid. LCMS m/z 952.6 (M+H), rt=1.03 min.

### Step 3: Synthesis of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[5-(piperazin-1-yl)pyrimidin-2-yl]benzene-1-sulfonamide (226c)

To a solution of tert-butyl 4-{2-[4-({6-[(3-cyclopropyl-1H-pyrazol-5-yl) (oxan-2-yl)amino]-5-methoxy-1,2-benzoxazol-3-yl}[(4-methoxyphenyl)methyl]sulfamoyl)-3,5-dimethoxyphenyl]pyrimidin-5-yl}piperazine-1-carboxylate (226b) (300 mg, 0.315 mmol) in dichloromethane (3 mL) and methanol (0.6 mL) was added 4M HCl in dioxane (9 mL). The reaction mixture was stirred at 15° C. for 16 hrs. The reaction mixture was filtered and concentrated to deliver a crude mixture of 226c (242 mg, >95% yield) which was a yellow solid and was used directly in the next step.

### Step 4: Synthesis of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[5-(4-methylpiperazin-1-yl)pyrimidin-2-yl]benzene-1-sulfonamide (226d)

To a suspension of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[5-(piperazin-1-yl)pyrimidin-2-yl]benzene-1-sulfonamide (226c) (242 mg, 0.315 mmol) in methanol (2.5 mL) was added 37 wt % (aq.) formaldehyde solution (256 mg, 3.1 mmol) at 15° C. The reaction mixture was stirred at 15° C. for 20 mins. Sodium cyanoborohydride (99.0 mg, 1.58 mmol) was added to the reaction mixture. The reaction mixture was stirred at 15° C. for 20 min then filtered through celite and concentrated to give crude 226d (246 mg, >95% yield) as a yellow solid used directly in the next step.

### Step 5: Synthesis of N-{6-[(3-cyclopropyl-1H-pyrazol-5-yl)amino]-5-methoxy-1,2-benzoxazol-3-yl}-2,6-dimethoxy-4-[5-(4-methylpiperazin-1-yl)pyrimidin-2-yl]benzene-1-sulfonamide (Example 226)

To a solution of N-{6-[(3-cyclopropyl-1H-pyrazol-5-yl)amino]-5-methoxy-1,2-benzoxazol-3-yl}-2,6-dimethoxy-N-[(4-methoxyphenyl)methyl]-4-[5-(4-methylpiperazin-1-yl)pyrimidin-2-yl]benzene-1-sulfonamide (226d) (246 mg, 0.315 mmol) in dichloromethane (5 mL) was added trifluoroacetic acid (2.5 mL), and the reaction mixture was stirred at 15° C. for 16 hrs. The reaction mixture was concentrated then diluted with dimethylformamide (2.5 mL) and purified using prep HPLC (C18 150×40 mm, 17-37% ACN: H₂O (modified with ammonium formate) over 9 min, flow rate 30 ml/mins). The product fractions were combined and lyophilized to give 226 (50 mg) with remaining impurities. This material was then purified using preparative HPLC (C18 150×40 mm, 5 μm 0-30% ACN: H₂O (modified with ammonium formate) over 9 min, flow rate 25 ml/mins) to deliver 226 (7.88 mg, 4% yield) as a white solid. ¹H NMR (400 MHZ, CDCl₃-d) δ 8.33 (s, 2H), 7.66 (s, 1H), 7.57 (s, 2H), 7.39 (s, 1H), 6.89 (s, 1H), 5.61 (s, 1H), 3.94 (s, 6H), 3.90 (s, 3H), 3.33.2 (m, 4H), 2.6-2.5 (m, 4H), 2.30 (s, 3H), 1.8-1.7 (m, 1H), 1.0-0.8 (m, 2H), 0.7-0.6 (m, 2H); LCMS m/z 662.4 (M+H)⁺; rt=0.778.

### Example 227: N-{6-[(3-cyclopropyl-1H-pyrazol-5-yl)amino]-5-methoxy-1,2-benzoxazol-3-yl}-4-{4-[1-(dimethylamino)ethyl]pyrimidin-2-yl}-2,6-dimethoxybenzene-1-sulfonamide

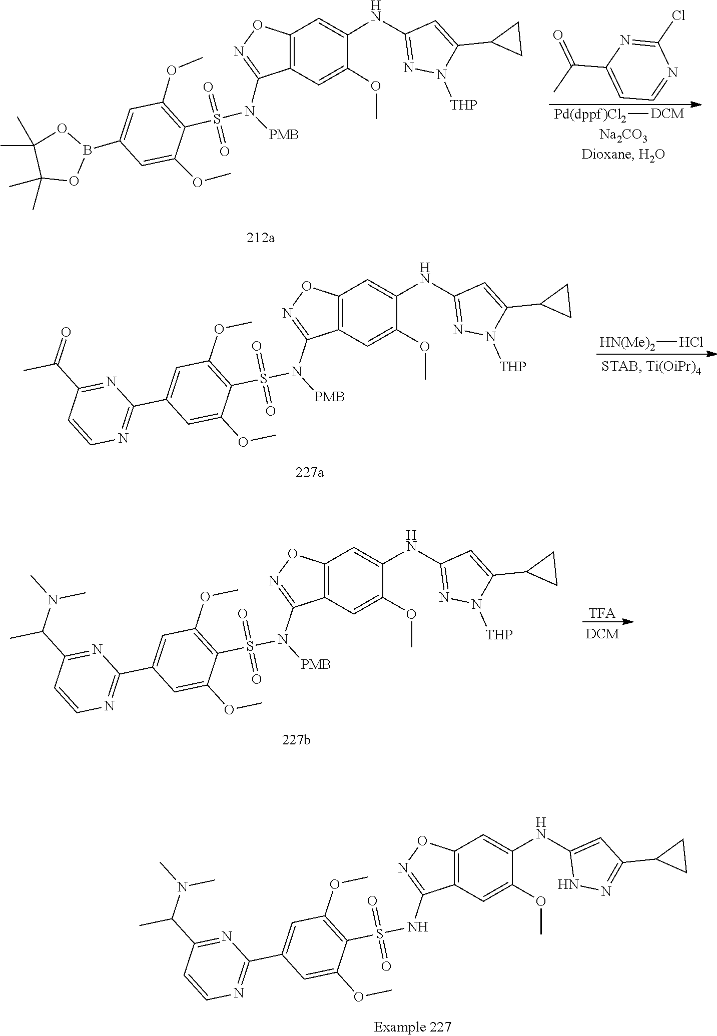

### Step 1: Synthesis of 4-(4-acetylpyrimidin-2-yl)-N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]benzene-1-sulfonamide (227a)

To a solution of 212a (1040 mg, 1.15 mmol) in dioxane (10 mL) and H₂O (2.5 mL) was added 1-(2-chloropyrimidin-4-yl) ethan-1-one (150 mg, 0.958 mmol), Na₂CO₃ (457 mg, 4.31 mmol) and Pd(dppf)Cl₂ complexed with DCM (1:1) (78 mg, 0.0958 mmol). The mixture was degassed and purged with nitrogen, then heated in a microwave to 100° C. for 2 hrs. The crude reaction was diluted with DCM, poured into water then extracted with DCM three times. The combined organic extracts were washed with water and brine, then dried over Na₂SO₄, filtered and concentrated. The crude residue was purified by column chromatography (SiO₂, 0-80% EtOAc/Pet. ether) to afford 227a (554 mg, 82%) as a yellow solid. LCMS m/z 810.4 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-4-{4-[1-(dimethylamino)ethyl]pyrimidin-2-yl}-2,6-dimethoxy-N-[(4-methoxyphenyl)methyl]benzene-1-sulfonamide (227b)

To a solution of 227a (500 mg, 0.617 mmol) and N,N-dimethylamine hydrochloride (65.4 mg, 0.803 mmol) in dichloroethane (6.17 mL) was added TEA (500 mg, 4.94 mmol). The reaction mixture was treated with titanium (IV) isopropoxide (439 mg, 1.23 mmol) and stirred at rt for 3 hrs. STAB (393 mg, 1.85 mmol) was added in portions and stirring was continued for 16 hrs. The reaction diluted with DCM and sat. aq. ammonium chloride then filtered through a celite pad and rinsed with DCM. The filtrate was washed with brine, then dried over MgSO₄ and concentrated. The crude residue was purified by column chromatography (SiO₂, 0-10% MeOH/DCM) to give 227b (420 mg, 81%) as a yellow solid. LCMS m/z 839.5 (M+H)⁺.

### Step 3: Synthesis of N-{6-[(3-cyclopropyl-1H-pyrazol-5-yl)amino]-5-methoxy-1,2-benzoxazol-3-yl}-4-{4-[1-(dimethylamino)ethyl]pyrimidin-2-yl}-2,6-dimethoxybenzene-1-sulfonamide (Example 227)

To a solution of 227b (420 mg, 0.501 mmol) in DCM (6.0 mL) and TFA (6.0 mL) was stirred at 15° C. for 8 hrs. The crude reaction was concentrated and purified by reverse phase HPLC (13-53% acetonitrile/water plus formic acid; 60 mL/min flow rate in 9 mins; Phenomenex Gemini NX 150×30 mm, 5 μm) to afford Example 227 (87 mg, 28%) as a white solid. ¹H NMR (400 MHz, DMSO-d₆) δ 8.95 (d, J=5.14 Hz, 1H) 8.14 (s, 1H) 8.07 (d, J=11.49 Hz, 2H) 7.73 (s, 2H) 7.58 (d, J=5.14 Hz, 1H) 7.43 (s, 1H) 5.77 (s, 1H) 3.89 (s, 9H) 2.45 (br s, 6H) 1.89-1.78 (m, 1H) 1.46 (br. d, J=6.60 Hz, 3H) 0.96-0.86 (m, 2H) 0.68-0.61 (m, 2H); LCMS m/z 635.4 (M+H)⁺.

### Examples 228 and 229: N-{6-[(3-cyclopropyl-1H-pyrazol-5-yl)amino]-5-methoxy-1,2-benzoxazol-3-yl}-4-(5,5-difluoro-1-methylpiperidin-3-yl)-2,6-dimethoxybenzene-1-sulfonamide

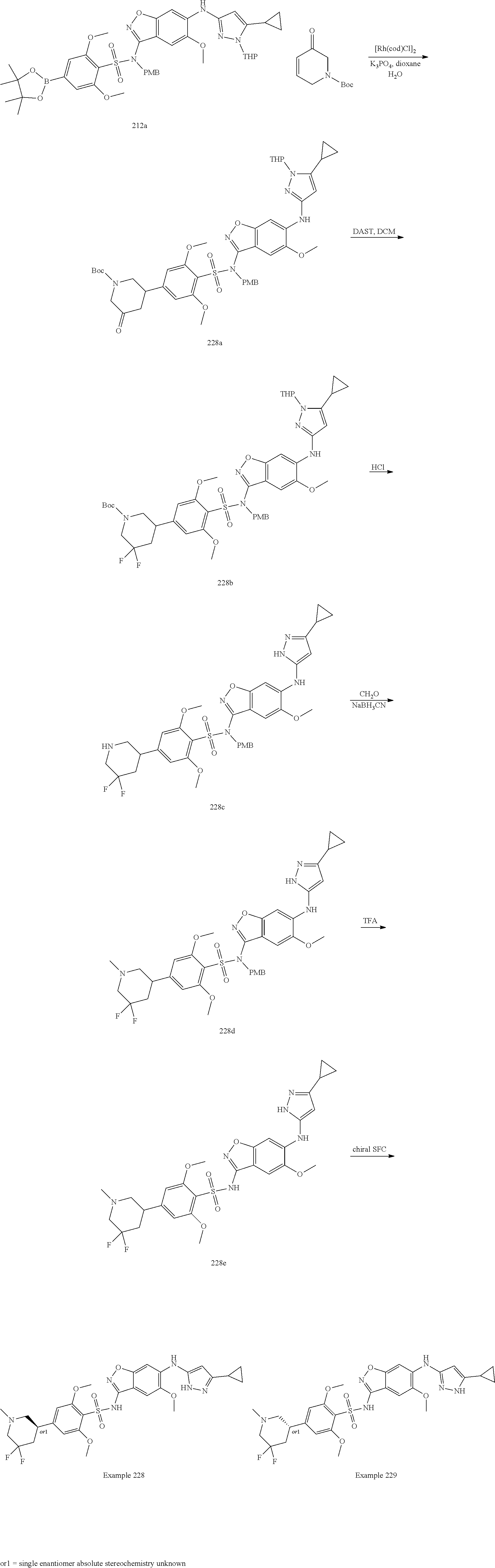

### Step 1: Synthesis of tert-butyl 3-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)-5-oxopiperidine-1-carboxylate (228a)

To a solution of tert-butyl 3-oxo-3,6-dihydropyridine-1 (2H)-carboxylate (250 mg, 1.27 mmol) and N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(4,4,5,5-tetramethyl-1,3,2-dioxaborolan-2-yl)benzene-1-sulfonamide (212a) (1590 mg, 1.95 mmol) in dioxane (5 mL) was added potassium phosphate (tribasic) (323 mg, 1.52 mmol), water (1 mL), and chloro (1,5-cyclooctadiene) rhodium (I) dimer (31 mg, 0.0634 mmol). The mixture was stirred under nitrogen at 25° C. for 3 hrs. The mixture was diluted with ethyl acetate (30 mL) and H₂O (20 mL). The organic layer was separated, washed with brine (20 mL), concentrated under reduced pressure. The residue was purified by column chromatography (SiO₂, Petroleum ether/Ethyl acetate=100/1 to 1/2) to give 228a (680 mg, 60.5% yield) as a yellow solid and mixture of enantiomers. LCMS m/z 887.4 (M+H), rt=2.121 min.

### Step 2: Synthesis of tert-butyl 5-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)-3,3-difluoropiperidine-1-carboxylate (228b)

To a solution of tert-butyl 3-(4-{(6-{[5-cyclopropyl-1-(oxan-2-yl)-1H-pyrazol-3-yl]amino}-5-methoxy-1,2-benzoxazol-3-yl) [(4-methoxyphenyl)methyl]sulfamoyl}-3,5-dimethoxyphenyl)-5-oxopiperidine-1-carboxylate (228a) (680 mg, 0.767 mmol) in dichlromethane (7.7 mL) at 0-5° C. was added diethylaminosulfur trifluoride (247 mg, 1.54 mmol). The reaction was warmed to 20° C. and stirred for 1 hr. The reaction mixture was quenched with sat. aq. NaHCO₃ (26 mL) and extracted with dichloromethane (3×20 mL). The combined organic extracts were washed with brine (50 mL), dried, filtered, and concentrated. The crude mixture was purified by biotage (20 g, EA/PE=0% to 60%) to give 228b (580 mg, 83% yield) as a yellow solid and mixture of enantiomers. LCMS m/z 909.5 (M+H)⁺, rt=1.113 min.

### Step 3: Synthesis of N-{6-[(5-cyclopropyl-1H-pyrazol-3-yl)amino]-5-methoxy-1,2-benzoxazol-3-yl}-4-(5,5-difluoropiperidin-3-yl)-2,6-dimethoxy-N-[(4-methoxyphenyl)methyl]benzene-1-sulfonamide (228c)

To a solution of tert-butyl 5-(4-{(6-{[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxyphenyl)-3,3-difluoropiperidine-1-carboxylate (228b) (580 mg, 0.64 mmol) in anhydrous MeOH (3 mL) at 15° C. was added 2M HCl in dioxane (9 mL). The reaction mixture was stirred at 15° C. for 16 hrs. The reaction mixture was concentrated to give crude 228c (510 mg, >95% yield) as a mixture of enantiomers, which was used directly in the next step. LCMS m/z 725.2 (M+H)⁺, rt=0.847.

### Step 4: Synthesis of N-{6-[(5-cyclopropyl-1H-pyrazol-3-yl)amino]-5-methoxy-1,2-benzoxazol-3-yl}-4-(5,5-difluoro-1-methylpiperidin-3-yl)-2,6-dimethoxy-N-[(4-methoxyphenyl)methyl]benzene-1-sulfonamide (228d)

To a suspension of N-{6-[(5-cyclopropyl-1H-pyrazol-3-yl)amino]-5-methoxy-1,2-benzoxazol-3-yl}-4-(5,5-difluoropiperidin-3-yl)-2,6-dimethoxy-N-[(4-methoxyphenyl)methyl]benzene-1-sulfonamide (228c) (500 mg, 0.627 mmol) in methanol (20.85 mL) was added 37 wt % (aq) formaldehyde solution (254 mg, 3.13 mmol) at 15° C. The reaction mixture was stirred at 15° C. for 10 mins. Sodium cyanoborohydride (98.5 mg, 1.57 mmol) was added to the reaction mixture. The reaction mixture was stirred at 15° C. for 10 min then filtered through celite and concentrated to give crude 228d (550 mg, >95% yield) which was used directly in the next step. LCMS m/z 739.3 (M+H)⁺, rt=0.898 min.

### Step 5: Synthesis of N-{6-[(5-cyclopropyl-1H-pyrazol-3-yl)amino]-5-methoxy-1,2-benzoxazol-3-yl}-4-(5,5-difluoro-1-methylpiperidin-3-yl)-2,6-dimethoxybenzene-1-sulfonamide (228e)

To a solution of N-{6-[(5-cyclopropyl-1H-pyrazol-3-yl)amino]-5-methoxy-1,2-benzoxazol-3-yl}-4-(5,5-difluoro-1-methylpiperidin-3-yl)-2,6-dimethoxy-N-[(4-methoxyphenyl)methyl]benzene-1-sulfonamide (228d) (500 mg, 0.677 mmol) in dichloromethane (19.8 mL) was added trifluoroacetic acid (6.6 mL). The reaction mixture was stirred at 15° C. for 16 hrs. The reaction mixture was concentrated and purified by preparative HPLC (C18 150×40 mm, 21-41% ACN: H₂O (modified with ammonium formate) over 9 min, flow rate 30 ml/mins). The product was lyophilized overnight to give 228e (71 mg, 17%) as a solid and mixture of enantiomers. LCMS m/z 619.3 (M+H)⁺, rt=0.883. The enantiomers were separated by chiral SFC (Daicel Chiralpak AD 250×30 mm, 10 μm, eluting with CO₂/EtOH (modified with 0.1% ammonium hydroxide) at a flow rate of 150 mL/mins) to afford N-{6-[(5-cyclopropyl-1H-pyrazol-3-yl)amino]-5-methoxy-1,2-benzoxazol-3-yl}-4-(5,5-difluoro-1-methylpiperidin-3-yl)-2,6-dimethoxybenzene-1-sulfonamide (Example 228) (18 mg, 51% yield, >99% ee) as a white solid and the first eluting peak. ¹H NMR (400 MHZ, METHANOL-d₄) δ 7.53 (br. d, J=2.00 Hz, 1H) 7.21 (s, 1H) 6.54 (s, 2H) 5.65 (s, 1H) 3.85 (s, 3H) 3.75 (s, 6H) 3.06-2.91 (m, 2H) 2.80 (br. d, J=12.01 Hz, 1H) 2.36-2.20 (m, 4H) 2.19-2.05 (m, 2H) 2.05-1.85 (m, 1H) 1.84-1.74 (m, 1H) 0.92-0.83 (m, 2H) 0.68-0.59 (m, 2H); LCMS m/z 619.3 (M+H)⁺, rt=0.883. [a]_{D}²²=−2° (c 1.0 mg/mL, CH₃OH).

### N-{6-[(5-cyclopropyl-1H-pyrazol-3-yl)amino]-5-methoxy-1,2-benzoxazol-3-yl}-4-(5,5-difluoro-1-methylpiperidin-3-yl)-2,6-dimethoxybenzene-1-sulfonamide (Example 229)

(16.7 mg, 47% yield, 97% ee) as a white solid and the second eluting peak. ¹H NMR (400 MHZ, METHANOL-d₄) δ 7.64 (br. s, 1H) 7.33 (s, 1H) 6.66 (s, 2H) 5.77 (s, 1H) 3.97 (s, 3H) 3.90-3.84 (m, 6H) 3.20-3.02 (m, 2H) 2.91 (br. d, J=11.51 Hz, 1H) 2.46-2.31 (m, 4H) 2.31-2.16 (m, 2H) 2.16-1.97 (m, 1H) 1.96-1.83 (m, 1H) 1.07-0.91 (m, 2H) 0.79-0.72 (m, 2H); LCMS m/z 619.3 (M+H)⁺, rt=0.883. [a]_{D}²²=+19.33° (c 1.0 mg/mL, CH₃OH).

### Example 230: N-{6-[(3-ethyl-1H-pyrazol-5-yl)amino]-5-methoxy-1,2-benzoxazol-3-yl}-2,6-dimethoxy-4-(1-methyl-1H-pyrazol-3-yl)benzene-1-sulfonamide

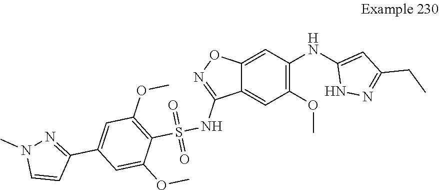 [SMILES: CCc1cc(Nc2cc3onc(NS(=O)(=O)c4c(OC)cc(-c5ccn(C)n5)cc4OC)c3cc2OC)[nH]n1]

Example 230 was made according to Method E using 4-bromo-N-[6-({5-ethyl-1-[(4-methoxyphenyl)methyl]-1H-pyrazol-3-yl}amino)-5-methoxy-1,2-benzoxazol-3-yl]-2,6-dimethoxybenzene-1-sulfonamide in place of 7a, ¹H NMR (400 MHZ, DMSO-d₆) δ 11.88 (s, 1 H), 10.97 (br s, 1H) 8.18-8.07 (m, 2H), 7.77 (d, J=2.32 Hz, 1H), 7.42 (s, 1H), 7.08 (s, 2H), 6.87 (d, J=2.32 Hz, 1H), 5.88 (s, 1H), 3.88 (d, J=3.18 Hz, 6H), 3.82 (s, 6H), 2.55 (q, J=7.82 Hz, 2H), 1.17 (t, J=7.64 Hz, 3H);LCMS m/z 554.3 (M+H)⁺

### Examples 231-238 were Made in a Library Format

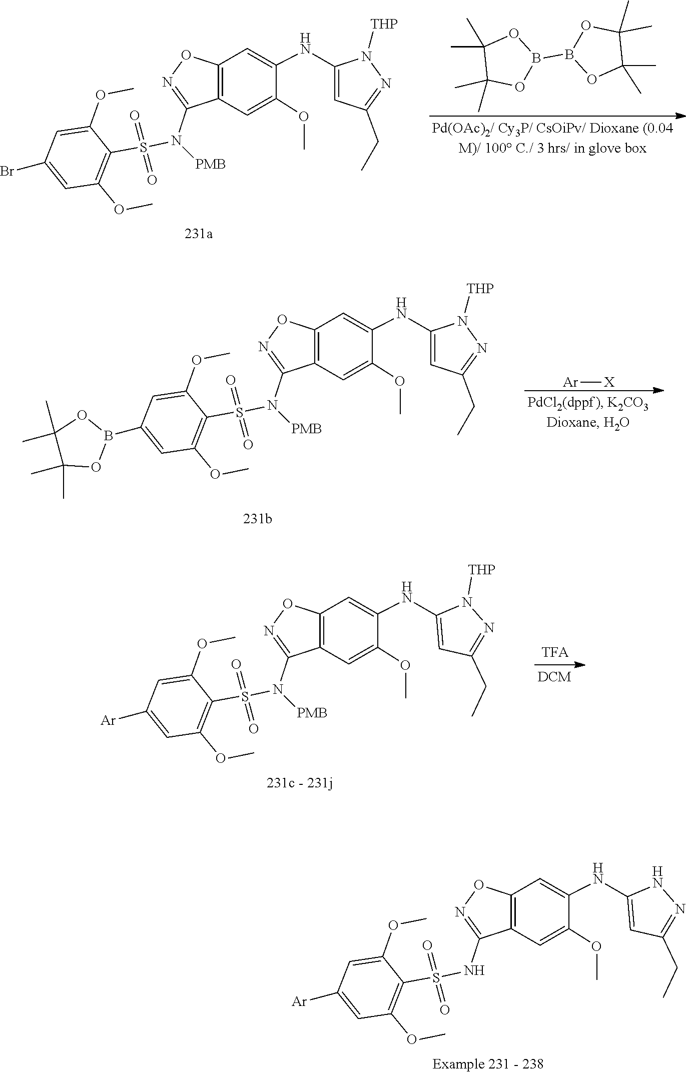

231a was prepared in a similar manner as 144a using 3-ethyl-1-(tetrahydro-2H-pyran-2-yl)-1H-pyrazol-5-amine in place of Intermediate N to prepare 143a. 3-ethyl-1-(tetrahydro-2H-pyran-2-yl)-1H-pyrazol-5-amine was prepared in a similar manner as Intermediate N using 5-ethyl-1H-pyrazol-3-amine in place of 5-cyclopropyl-1H-pyrazol-3-amine.

### Protocol for the Synthesis of Library Compounds

Step 1: Prepare a 0.04 M solution of 231a in dioxane, dispense 500 ul (20 umol, 1.0 eq.) of solution to 4 mL reaction vials in glove box. Dispense bis (pinacolato)diboron (30 umol, 1.5 eq.) to above vials, followed by cesium pivalate (60 umol, 3.0 eq.), Pd (OAc) 2 (2 umol, 0.1 eq.) and tricyclohexylphosphine (4 umol, 0.2 eq.). Cap vials and transfer out of the glove box. Stir the mixtures at 100° C. for 3 hrs. Evaporate solvent by Speed vacuum. Wash the mixture with H₂O (0.5 mL) and extract with EtOAc (0.75 mL×2). Collect organic layer and concentrate to give crude intermediate 231b which was used for next step directly.

Step 2: A 1.5 M solution of K₂CO₃ in H₂O was prepared. Dispense 200 ul of dioxane to 8 mL reaction vials containing 231b (20 umol, 1.0 eq.) in glove box. Dispense each ArX (30 umol, 1.5 eq.) to above vials. Dispense 40 ul (60 umol, 3.0 eq.) of K₂CO₃ solution to each vial. Dispense Pd(dppf)Cl₂ to each vial. Cap vials and transfer out of the glove box. Shake at 100° C. for 16 hrs. Evaporate solvent by Speed vacuum. Wash the mixture with of H₂O (0.5 mL) and extract with EtOAc (0.75 mL×2). Collect organic layer, dry over anhydrous Na₂SO₄, filter and concentrate the filtrate to give crude intermediate 231c-231j which was used for next step directly. Step 3: Prepare a 1:1 solution of DCM/TFA. Dispense 0.4 mL of TFA solution to 8 mL reaction vials containing intermediates 231c-231j (20 umol, 1.0 eq.). Cap vials and shake at 30° C. for 8 hrs. Remove solvent by Speed vacuum. Purify residue by preparative HPLC (acetonitrile/water plus 0.225% formic acid; 25 mL/min; Boston Prime C18 150×30 mm×5 mm) to give Examples 231-238.

TABLE 24

| Example | | |
|---|---|---|
| Number | Structure/IUPAC Name | LCMS data |
| 231 | 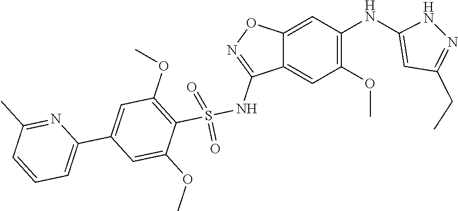 [SMILES: CCc1cc(Nc2cc3onc(NS(=O)(=O)c4c(OC)cc(-c5cccc(C)n5)cc4OC)c3cc2OC)[nH]n1] | LCMS m/z 565.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(6-methylpyridin-2- | |
| | yl)benzene-1-sulfonamide | |
| 232 | 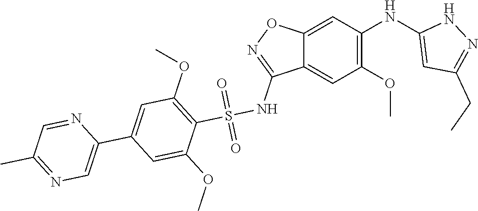 [SMILES: CCc1cc(Nc2cc3onc(NS(=O)(=O)c4c(OC)cc(-c5cnc(C)cn5)cc4OC)c3cc2OC)[nH]n1] | LCMS m/z 566.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(5-methylpyrazin-2- | |
| | yl)benzene-1-sulfonamide | |
| 233 | 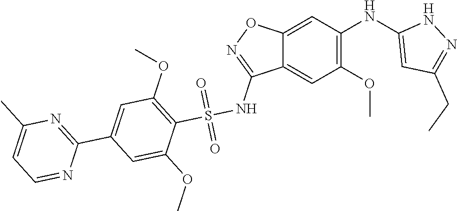 [SMILES: CCc1cc(Nc2cc3onc(NS(=O)(=O)c4c(OC)cc(-c5nccc(C)n5)cc4OC)c3cc2OC)[nH]n1] | LCMS m/z 566.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(4-methylpyrimidin-2- | |
| | yl)benzene-1-sulfonamide | |
| 234 | 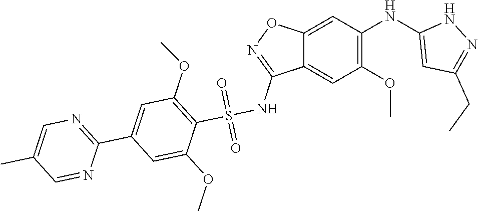 [SMILES: CCc1cc(Nc2cc3onc(NS(=O)(=O)c4c(OC)cc(-c5ncc(C)cn5)cc4OC)c3cc2OC)[nH]n1] | LCMS m/z 566.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(5-methylpyrimidin-2- | |
| | yl)benzene-1-sulfonamide | |
| 235 | 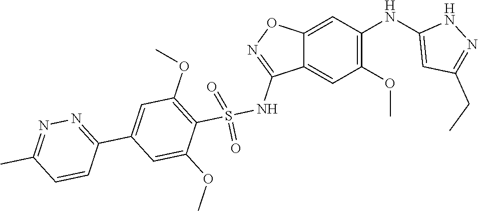 [SMILES: CCc1cc(Nc2cc3onc(NS(=O)(=O)c4c(OC)cc(-c5ccc(C)nn5)cc4OC)c3cc2OC)[nH]n1] | LCMS m/z 566.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(6-methylpyridazin-3- | |
| | yl)benzene-1-sulfonamide | |
| 236 | 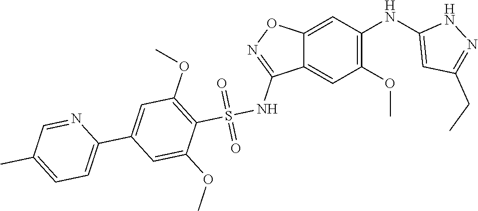 [SMILES: CCc1cc(Nc2cc3onc(NS(=O)(=O)c4c(OC)cc(-c5ccc(C)cn5)cc4OC)c3cc2OC)[nH]n1] | LCMS m/z 565.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(5-methylpyridin-2- | |
| | yl)benzene-1-sulfonamide | |
| 237 | 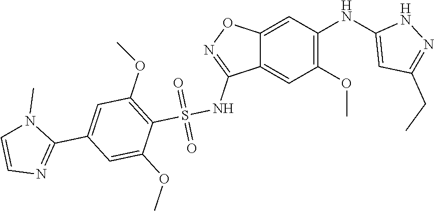 [SMILES: CCc1cc(Nc2cc3onc(NS(=O)(=O)c4c(OC)cc(-c5nccn5C)cc4OC)c3cc2OC)[nH]n1] | LCMS m/z 554.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(1-methyl-1H-imidazol-2- | |
| | yl)benzene-1-sulfonamide | |
| 238 | 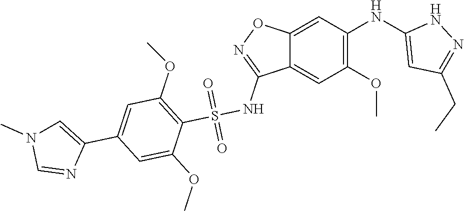 [SMILES: CCc1cc(Nc2cc3onc(NS(=O)(=O)c4c(OC)cc(-c5cn(C)cn5)cc4OC)c3cc2OC)[nH]n1] | LCMS m/z 554.2 (M + H)⁺ |
| | N-{6-[(3-ethyl-1H-pyrazol-5-yl)amino]- | |
| | 5-methoxy-1,2-benzoxazol-3-yl}-2,6- | |
| | dimethoxy-4-(1-methyl-1H-imidazol-4- | |
| | yl)benzene-1-sulfonamide | |

### Examples 239 and 240:4-(1,4-dioxan-2-yl)-N-{6-[(3-ethyl-1H-pyrazol-5-yl)amino]-5-methoxy-1.2-benzoxazol-3-yl}-2,6-dimethoxvbenzene-1-sulfonamide

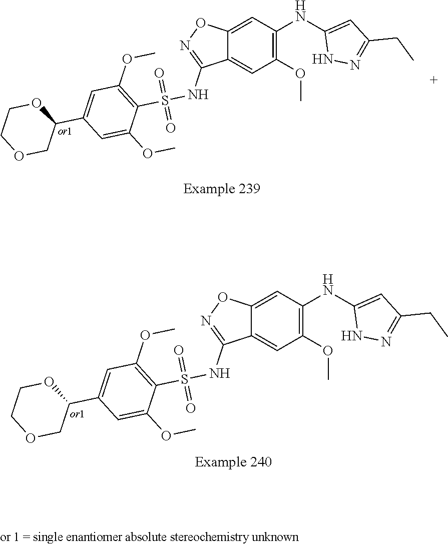 [SMILES: CCc1cc(Nc2cc3onc(NS(=O)(=O)c4c(OC)cc([C@H]5COCCO5)cc4OC)c3cc2OC)[nH]n1 ; CCc1cc(Nc2cc3onc(NS(=O)(=O)c4c(OC)cc([C@@H]5COCCO5)cc4OC)c3cc2OC)[nH]n1]

Examples 239 and 240 were prepared according to Method B using racemic 145c in place of 2,6-dimethoxybenzene-1-sulfonyl chloride. The enantiomers were separated by chiral SFC (Daicel Chiralcel OJ (250×30 mm, 10 μm) eluting with CO₂/iPrOH (modified with 0.1% ammonium hydroxide) isocratic at a flow rate of 80 mL/mins) to afford Example 239 4-(1,4-dioxan-2-yl)-N-{6-[(3-ethyl-1H-pyrazol-5-yl)amino]-5-methoxy-1,2-benzoxazol-3-yl}-2,6-dimethoxybenzene-1-sulfonamide (29.2 mg, 18% yield, >99% ee) as a white solid and the first eluting peak. ¹H NMR (400 MHZ, CDCl₃-d) δ 7.73 (s, 1H), 7.5-7.4 (m, 1H), 7.1-7.0 (m, 1H), 6.6-6.5 (m, 2H), 5.9-5.8 (m, 1H), 4.6-4.5 (m, 1H), 4.0-3.9 (m, 3H), 3.91 (s, 6H), 3.9-3.8 (m, 4H), 3.7-3.7 (m, 1H), 3.34 (dd, J=10.2, 11.6 Hz, 1H), 2.68 (q, J=7.1 Hz, 2H), 1.30 (t, J=7.6 Hz, 3H); LCMS m/z 560.2 (M+H)⁺; rt=0.852. [a]_{D}22=−168.7° (c 1.0, MeOH), and 4-(1,4-dioxan-2-yl)-N-{6-[(3-ethyl-1H-pyrazol-5-yl)amino]-5-methoxy-1,2-benzoxazol-3-yl}-2,6-dimethoxybenzene-1-sulfonamide (Example 240) (44.0 mg, 28% yield, 99% ee) as a white solid and the second eluting peak. ¹H NMR (400 MHZ, CHLOROFORM-d) δ 7.8-7.7 (m, 1H), 7.5-7.4 (m, 1H), 7.07 (br. s, 1H), 6.58 (s, 2H), 5.9-5.8 (m, 1H), 4.56 (dd, J=2.6, 9.9 Hz, 1H), 4.0-3.9 (m, 3H), 3.92 (s, 6H), 3.9-3.8 (m, 4H), 3.7-3.7 (m, 1H), 3.4-3.3 (m, 1H), 2.7-2.6 (m, 2H), 1.30 (t, J=7.6 Hz, 3H); LCMS m/z 560.3 (M+H)⁺; rt=0.852. [a]_{D}²²=159.33° (c 1.0, MeOH).

### Examples 241 and 242: 2,6-dimethoxy-N-(5-methoxy-6-{[3-(2-methylcyclopropyl)-1H pyrazol-5-yl]amino}-1,2-benzoxazol-3-yl)-4-(1-methyl-1H-pyrazol-3-yl)benzene-1-sulfonamide

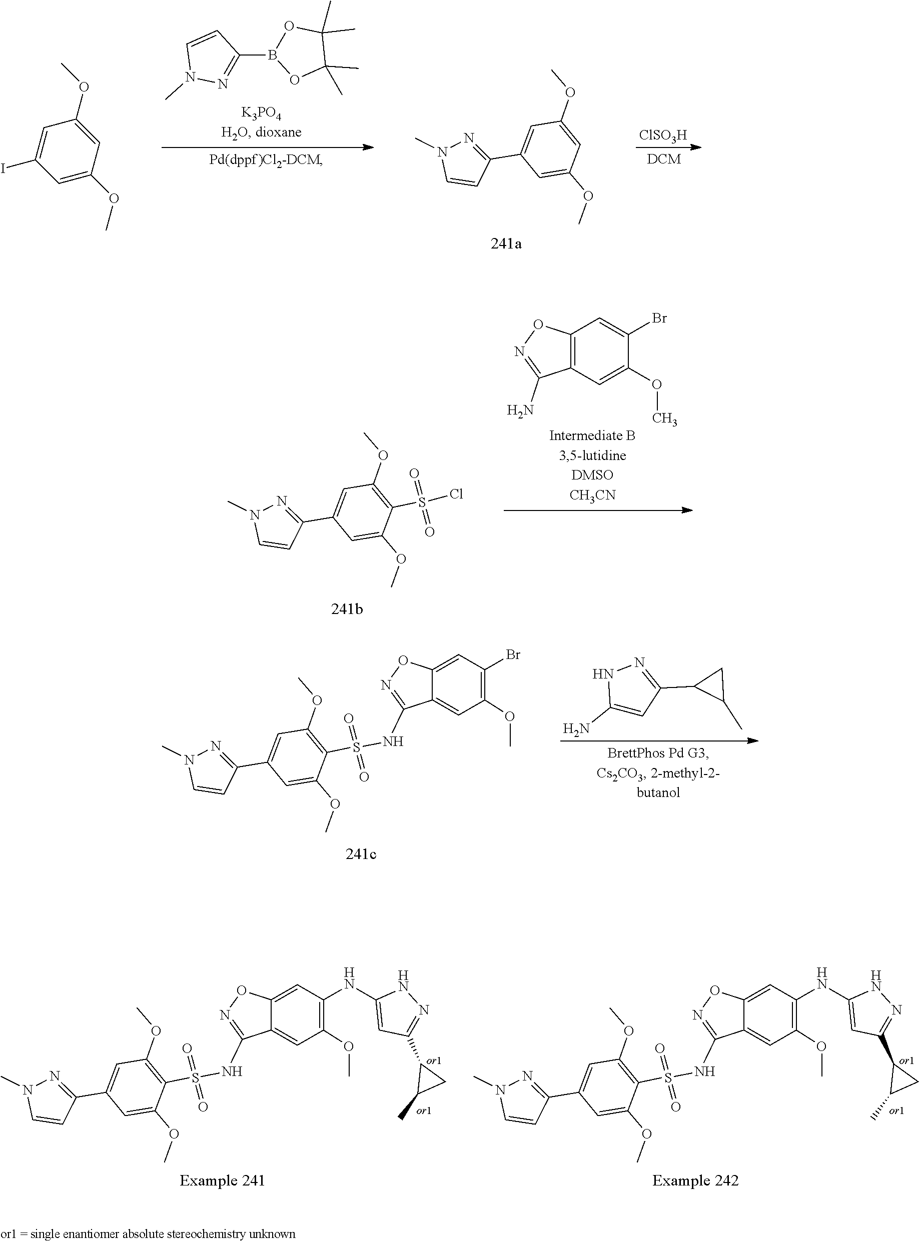

### Step 1: Synthesis of 3-(3,5-dimethoxyphenyl)-1-methyl-1H-pyrazole (241a)

To a solution of 1-iodo-3,5-dimethoxybenzene (9000 mg, 34.08 mmol) and 1-methyl-1H-pyrazole-3-boronic acid pinacol ester (7090 mg, 34.1 mmol) in dioxane (140 mL) and H₂O (30 mL) under nitrogen was added tripotassium phosphate (21700 mg, 102 mmol) and [1,1′-bis(diphenylphosphino)ferrocene]dichloropalladium (II), complex with dichloromethane (2490 mg, 3.41 mmol). The reaction mixture was stirred at 80° C. for 30 mins and was then concentrated under reduced pressure. The resulting crude was purified by flash silica gel column chromatography (eluting with 0-35% EtOAC in Pet. ether) to give 3300 mg of 241a as a yellow oil and 5000 mg of 241a with impurities (purity ˜60%). The latter was repurified by preparative HPLC (C18 150×40 mm column, Mobile phase A: H₂O+NH₃H₂O+NH₄HCO₃ Mobile phase B: ACN 26-66% over 9 min, Flow rate 60 mL/min) to give 241a (2500 mg) as a grey solid. The combined batches afforded 241a (5800 mg, 78%), ¹H NMR (400 MHZ, CDCl₃) δ 7.41-7.36 (m, 1H), 6.99-6.95 (m, 2H), 6.54-6.51 (m, 1H), 6.47-6.39 (m, 1H), 3.99-3.95 (m, 3H), 3.88-3.83 (m, 6H), LCMS m/z 218.9 (M+H)⁺.

### Step 2: Synthesis of 2,6-dimethoxy-4-(1-methyl-1H-pyrazol-3-yl)benzene-1-sulfonyl chloride (241b)

To a solution of 3-(3,5-dimethoxyphenyl)-1-methyl-1H-pyrazole 241a (1000 mg, 4.582 mmol) in DCM (20 mL) at 0° C. was added chlorosulfonic acid (5 mL). The reaction mixture was stirred at at that temperature for 1.5 hrs and was then slowly added to ice water. The aqueous phase was extracted with DCM (20 mL×3). The combined organic phase was washed with brine, dried over sodium sulfate, filtered, and concentrated under reduced pressure. The crude 241b (1050 mg) was obtained as a brown solid and was used in the next step without further purification. LCMS m/z 317.0 (M+H)⁺.

### Step 3: Synthesis of N-(6-bromo-5-methoxy-1,2-benzoxazol-3-yl)-2,6-dimethoxy-4-(1-methyl-1H-pyrazol-3-yl)benzene-1-sulfonamide (241c)

To a mixture of Intermediate B (500 mg, 2.06 mmol) and 2,6-dimethoxy-4-(1-methyl-1H-pyrazol-3-yl)benzene-1-sulfonyl chloride 241b (1050 mg, 3 mmol) in MeCN (4.11 mL) was added 3,5-lutidine (882 mg, 8.23 mmol), followed by a 0.05 M solution of DMSO in ACN (2.06 mL). The reaction mixture was stirred at 60° C. for 16 hrs and was then concentrated under reduced pressure. The resulting residue was purified by flash silica gel column chromatography (eluting with 0-80% EtOAc in Pet. ether) to give the desired product 241c (290 mg, 29%) as a yellow solid. ¹H NMR (400 MHZ, CDCl₃) δ 7.74-7.67 (m, 1H), 7.48-7.44 (m, 1H), 7.40-7.36 (m, 1H), 6.56-6.53 (m, 1H), 6.51-6.47 (m, 1H), 6.31-6.26 (m, 1H), 3.95-3.89 (m, 6H), 3.88-3.80 (m, 6H); LCMS m/z 524.9 (M+H)⁺. And side product N-(6-bromo-5-methoxy-1,2-benzoxazol-3-yl)-2,4-dimethoxy-6-(1-methyl-1H-pyrazol-3-yl)benzene-1-sulfonamide (310 mg, 27%) as a yellow solid. ¹H NMR (400 MHZ, CDCl₃) δ 7.72-7.64 (m, 2H), 7.45-7.39 (m, 1H), 7.07-7.02 (m, 2H), 6.58-6.53 (m, 1H), 4.03-3.99 (m, 9H), 3.99-3.96 (m, 3H); LCMS m/z 524.9 (M+H)⁺.

### Step 4: Synthesis of 2,6-dimethoxy-N-(5-methoxy-6-{[3-(2-methylcyclopropyl)-1H-pyrazol-5-yl]amino}-1,2-benzoxazol-3-yl)-4-(1-methyl-1H-pyrazol-3-yl)benzene-1-sulfonamide (Example 241 and 242)

A yellow suspension of N-(6-bromo-5-methoxy-1,2-benzoxazol-3-yl)-2,6-dimethoxy-4-(1-methyl-1H-pyrazol-3-yl)benzene-1-sulfonamide 241c (240 mg, 0.459 mmol), 5-(2-Methylcyclopropyl)-1H-pyrazol-3-amine (2 M in dioxane, 126 mg, 0.917 mmol), cesium carbonate (448 mg, 1.38 mmol) and BrettPhos Pd G3 (83.1 mg, 0.0917 mmol) in 2-methyl-2-butanol (4.59 mL) was degassed six times with nitrogen and was stirred at 100° C. for 1 hr. The reaction mixture was allowed to cool to rt and combined with a smaller batch (100 mg of 241c was used). EtOAc was added and the mixture was filtered and concentrated under reduced pressure. The resulting residue was purified by flash silica gel column chromatography (eluting with 0-54% EtOAc in Pet. ether followed by 1-10% MeOH in DCM) to afford a racemic mixture of 241 and 242 (180 mg, combined yield: 48%) as a yellow gum. LCMS m/z 580.2 (M+H)⁺. The material was submitted for chiral separation by SFC (80% ethanol with 0.1% ammonium hydroxide in heptane; 80 mL/min flow rate; Column: Daicel Chiralpak® IG (250×30 mm; 10 □m)) but led to a mixture of the 2 enantiomers 241 and 242 (117 mg) as a white solid. ¹H NMR (400 MHZ, CDCl₃) δ 7.71-7.68 (m, 1H), 7.49-7.46 (m, 1H), 7.42-7.39 (m, 1H), 7.03-7.02 (m, 2H), 7.01-6.97 (m, 1H), 6.57-6.51 (m, 1H), 5.68-5.64 (m, 1H), 4.00-3.94 (m, 12H), 1.57-1.50 (m, 1H), 1.22 (br. s, 3H), 1.15-1.08 (m, 1H), 0.97-0.89 (m, 1H), 0.81-0.74 (m, 1H); LCMS m/z 580.2 (M+H)⁺. The enantiomers were separated by chiral SFC (column: Chiralpak IK (21 mm×250 mm; 5 μm), Mobile phase A: CO₂ Mobile phase B: MeOH+10 mm NH₃ 28% B isocratic @ 100 bar, 100 mL/min flow rate).

### 2,6-dimethoxy-N-(5-methoxy-6-{[3-(2-methylcyclopropyl)-1H-pyrazol-5-yl]amino}-1,2-benzoxazol-3-yl)-4-(1-methyl-1H-pyrazol-3-yl)benzene-1-sulfonamide (Example 241)

The first eluting peak was lyophilized and gave 241 (33.2 mg, 8.8%, >99% ee) as a white solid. ¹H NMR (400 MHZ, DMSO-d₆) δ 11.82 (br s, 1H), 11.03-10.83 (m, 1H), 8.09 (br s, 1H), 8.03-7.92 (m, 1H), 7.76 (d, J=2.2 Hz, 1H), 7.40 (s, 1H), 7.08 (s, 2H), 6.86 (d, J=2.4 Hz, 1H), 5.74 (s, 1H), 3.89 (s, 3H), 3.87 (s, 3H), 3.82 (s, 6H), 1.55 (td, J=4.5, 8.7 Hz, 1H), 1.14-1.08 (m, 3H), 1.08-1.00 (m, 1H), 0.89-0.79 (m, 1H), 0.79-0.63 (m, 1H); LCMS m/z 580.3 (M+H)⁺; [a],22=−13.4° (c 0.1, MeOH)

### 2,6-dimethoxy-N-(5-methoxy-6-{[3-(2-methylcyclopropyl)-1H-pyrazol-5-yl]amino}-1,2-benzoxazol-3-yl)-4-(1-methyl-1H-pyrazol-3-yl)benzene-1-sulfonamide (Example 242)

The second eluting peak was lyophilized and gave 242 (35.6 mg, 8.8%, ˜90% ee) as an off-white powder. ¹H NMR (600 MHZ, DMSO-d₆) δ 11.82 (br. s, 1H), 10.92 (br. s, 1H), 8.09 (br. s, 1H), 7.98 (br. s, 1H), 7.76 (d, J=2.2 Hz, 1H), 7.40 (s, 1H), 7.08 (s, 2H), 6.86 (d, J=2.4 Hz, 1H), 5.74 (s, 1H), 3.89 (s, 3H), 3.87 (s, 3H), 3.82 (s, 6H), 1.55 (td, J=4.6, 8.6 Hz, 1H), 1.11 (d, J=5.8 Hz, 3H), 1.07-1.01 (m, 1H), 0.84 (td, J=4.5, 8.7 Hz, 1H), 0.70 (td, J=5.1, 9.1 Hz, 1H); LCMS m/z 580.3 (M+H)⁺; [a]_{D}²²=+21.8° (c 0.1, MeOH).

### Example 243: N-6-[(3-cyclopropyl-1H-pyrazol-5-yl)amino]-5-methoxy-1,2-benzoxazol-3-yl}-2,6-dimethoxy-4-(6-oxopyridazin-1(6H)-yl)benzene-1-sulfonamide

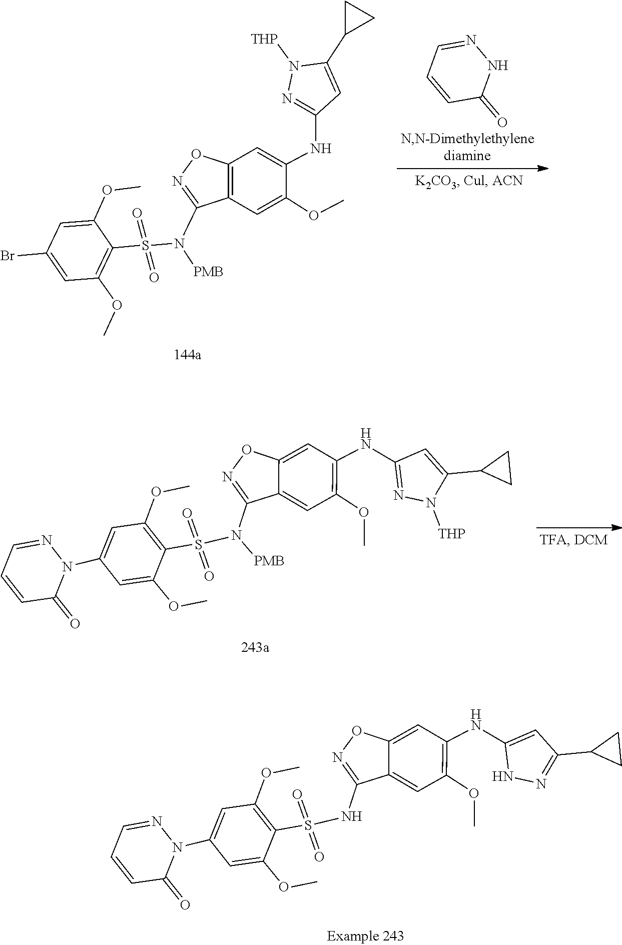

### Step 1: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(6-oxopyridazin-1(6H)-yl)benzene-1-sulfonamide (243a)

To a solution of 144a (500 mg, 0.651 mmol), 3(2H)-pyridazinone (62.5 mg, 0.650 mmol), potassium carbonate (198 mg, 1.43 mmol) and N,N-dimethylethylenediamine (28.7 mg, 0.325 mmol) in ACN (3.0 mL) was added copper iodide (31.0 mg, 0.163 mmol) at 25° C. The reaction was bubbled with nitrogen for 5 mins, then sealed in a microwave tube and irradiated in the microwave on a Biotage Smith Synthesizer at 120° C. for 2 hrs. The resulting mixture was concentrated under reduced pressure, and the crude residue was purified by flash column chromatography (24 g silica gel, 0-100% EtOAc/Pet. ether) to afford 243a (220 mg, 43% yield) as a brown solid. LCMS m/z 784.2 (M+H)⁺.

### Step 2: Synthesis of N-{6-[(3-cyclopropyl-1H-pyrazol-5-yl)amino]-5-methoxy-1,2-benzoxazol-3-yl}-2,6-dimethoxy-4-(6-oxopyridazin-1(6H)-yl)benzene-1-sulfonamide (Example 243)

A solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(6-oxopyridazin-1(6H)-yl)benzene-1-sulfonamide 243a (255 mg, 0.325 mmol) in DCM (1.0 mL) and TFA (2.0 mL) was stirred at 25° C. for 4 hrs. The reaction was concentrated under reduced pressure and the resulting crude residue was purified by preparative HPLC (C18 150×40 mm column, Mobile phase A: Water+NH₃H₂O+NH₄HCO₃ Mobile phase B: ACN 3-43% over 9 mins flow rate 60 mL/mins) to afford Example 243 (122 mg, 65% yield) as a white solid. ¹H NMR (400 MHZ, DMSO-d₆) δ 11.93-11.88 (m, 1H), 11.17-11.10 (m, 1H), 8.14 (s, 1H), 8.09-8.06 (m, 2H), 7.50 (dd, J=9.57, 3.85 Hz, 1H), 7.40-7.37 (m, 1H), 7.08 (dd, J=9.68, 1.54 Hz, 1H), 7.02-6.99 (m, 2H), 5.79-5.76 (m, 1H), 3.88 (s, 3H), 3.77 (s, 6H), 1.91-1.80 (m, 1H), 0.97-0.86 (m, 2H), 0.69-0.62 (m, 2H); LCMS m/z 580.3 (M+H)⁺.

### Examples 244 and 245: N-{6-[(3-cyclopropyl-1H-pyrazol-5-yl)amino]-5-methoxy-1,2-benzoxazol-3-yl}-2,6-dimethoxy-4-(2-methyl-5-oxopyrrolidin-1-yl)benzene-1-sulfonamide

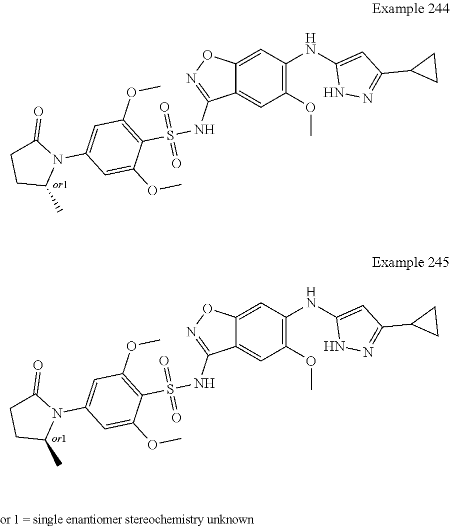 [SMILES: COc1cc2c(NS(=O)(=O)c3c(OC)cc(N4C(=O)CC[C@H]4C)cc3OC)noc2cc1Nc1cc(C2CC2)n[nH]1 ; COc1cc2c(NS(=O)(=O)c3c(OC)cc(N4C(=O)CC[C@@H]4C)cc3OC)noc2cc1Nc1cc(C2CC2)n[nH]1]

Examples 244 and 245 were made in a similar manner as Example 243 using 5-methylpyrrolidin-2-one in place of pyridazin-3(2H)-one. The racemic mixture was purified by reverse phase chromatography (C18 column, 150×40 mm, aq ammonium formate-acetonitrile mobile phase) to give 40 mg (25%) as a white solid. The enantiomers were separated by chiral SFC (Daicel Chiralpak AS 250×30 mm, 10 μm, 55% isopropanol with 0.1% ammonium hydroxide in CO₂; 80 mL/mins flow rate).

### N-{6-[(3-cyclopropyl-1H-pyrazol-5-yl)amino]-5-methoxy-1,2-benzoxazol-3-yl}-2,6-dimethoxy-4-(2-methyl-5-oxopyrrolidin-1-yl)benzene-1-sulfonamide (Example 244)

The first eluting peak was isolated as a white solid (10 mg, 6% yield). ¹H NMR (400 MHZ, DMSO-d₆) δ 11.90 (s, 1H), 10.92 (br. s, 1H), 8.12 (s, 1H), 8.03 (s, 1H), 7.40 (s, 1H), 6.95 (s, 2H), 5.78 (s, 1H), 4.60-4.46 (m, 1H), 3.87 (s, 3H), 3.75 (s, 6H), 2.64 (td, J=8.8, 17.1 Hz, 1H), 2.46-2.34 (m, 1H), 2.30-2.18 (m, 1H), 1.91-1.81 (m, 1H), 1.67 (tdd, J=4.5, 8.5, 12.7 Hz, 1H), 1.18 (d, J=6.1 Hz, 3H), 0.96-0.88 (m, 2H), 0.71-0.60 (m, 2H); LCMS m/z 583.2 (M+H)⁺; [a],26=−2° (c=0.1 g/100 mL, CH₃OH).

### N-{6-[(3-cyclopropyl-1H-pyrazol-5-yl)amino]-5-methoxy-1,2-benzoxazol-3-yl}-2,6-dimethoxy-4-(2-methyl-5-oxopyrrolidin-1-yl)benzene-1-sulfonamide (Example 245)

The second eluting peak was isolated as a white solid (10 mg, 6% yield). ¹H NMR (400 MHZ, DMSO-d₆) δ11.88 (br. s, 1H), 10.92 (br. d, J=2.2 Hz, 1H), 8.09 (s, 1H), 8.02-7.87 (m, 1H), 7.36 (br. s, 1H), 6.93 (s, 2H), 5.77 (s, 1H), 4.58-4.47 (m, 1H), 3.86 (s, 3H), 3.72 (s, 6H), 2.71-2.57 (m, 1H), 2.45-2.34 (m, 1H), 2.30-2.15 (m, 1H), 1.93-1.79 (m, 1H), 1.67 (tdd, J=4.5, 8.8, 12.9 Hz, 1H), 1.17 (d, J=6.2 Hz, 3H), 0.96-0.86 (m, 2H), 0.71-0.60 (m, 2H); LCMS m/z 583.2 (M+H)⁺; [a]_{D}²⁶=+22° (c=0.1 g/100 mL, CH₃OH).

### Example 246: N-{6-[(3-cyclopropyl-1H-pyrazol-5-yl)amino]-5-methoxy-1,2-benzoxazol-3-yl}-2,6-dimethoxy-4-(2-oxopyrrolidin-1-yl)benzene-1-sulfonamide

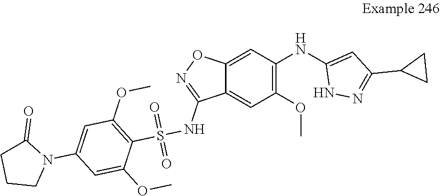 [SMILES: COc1cc2c(NS(=O)(=O)c3c(OC)cc(N4CCCC4=O)cc3OC)noc2cc1Nc1cc(C2CC2)n[nH]1]

Example 246 was made in a similar manner as Example 243 using pyrrolidin-2-one in place of 3(2H)-pyridazinone. ¹H NMR (400 MHZ, DMSO-d₆) δ 12.03-11.81 (m, 1H), 11.05-10.72 (m, 1H), 8.17-8.08 (m, 1H), 8.08-8.02 (m, 1H), 7.45-7.38 (m, 1H), 7.09-6.98 (m, 2H), 5.85-5.75 (m, 1H), 3.92-3.81 (m, 5H), 3.80-3.69 (m, 6H), 2.58-2.53 (m, 2H), 2.09-1.98 (m, 2H), 1.91-1.81 (m, 1H), 0.97-0.89 (m, 2H), 0.71-0.59 (m, 2H); LCMS m/z 569.3 (M+H)⁺.

### Example 247: N-{6-[(3-cyclopropyl-1H-pyrazol-5-yl)amino]-5-methoxy-1,2-benzoxazol-3-yl}-2,6-dimethoxy-4-[methyl (oxolan-3-yl)amino]benzene-1-sulfonamide

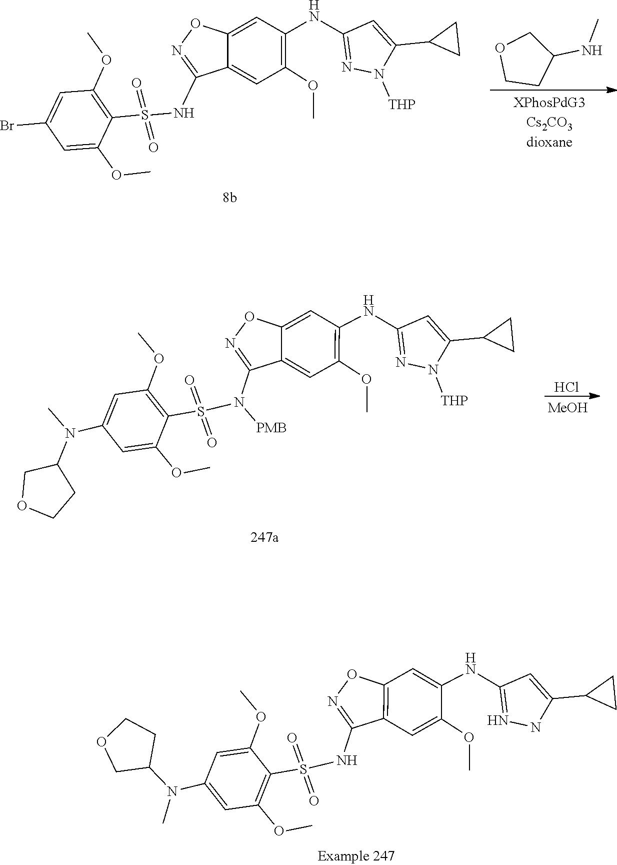

### Step 1: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-[methyl (oxolan-3-yl)amino]benzene-1-sulfonamide (247a)

To 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide (8b) (200 mg, 0.308 mmol) in 1,4-dioxane (5 mL) was added sequentially N-methyltetrahydrofuran-3-amine hydrochloride (84.9 mg, 0.617 mmol), Xphos Pd G3 (52 mg, 0.0617 mmol), and cesium carbonate (502 mg, 1.43 mmol). The mixture was degassed via nitrogen sparge and heated to 95° C. for 16 hrs. The mixture was cooled to rt, filtered, and concentrated under reduced pressure to 247a (40 mg, 20%) in crude form. The material was taken directly into the next step.

### Step 2: Synthesis of N-{6-[(3-cyclopropyl-1H-pyrazol-5-yl)amino]-5-methoxy-1,2-benzoxazol-3-yl}-2,6-dimethoxy-4-[methyl (oxolan-3-yl)amino]benzene-1-sulfonamide (Example 247)

To N-(6-{[5-cyclopropyl-1-(oxan-2-yl)-1H-pyrazol-3-yl]amino}-5-methoxy-1,2-benzoxazol-3-yl)-2,6-dimethoxy-4-[methyl (oxolan-3-yl)amino]benzene-1-sulfonamide 247a (40 mg, 0.060 mmol) in methanol (2.0 mL) was added a solution of 4.0 M HCl in 1,4-dioxane (2.0 mL) at rt. The mixture was stirred for 3 hrs and was then concentrated under reduced pressure. The residue was then diluted with water (2 mL) and the pH was adjusted to weakly alkaline by the addition of ammonium hydroxide (0.100 mL). The solution was extracted with ethyl acetate (2×2 mL), and the combined extracts were washed with brine (2 mL), dried over sodium sulfate, and concentrated. The residue was purified by reverse phase HPLC (C18 150×40 mm column, aqueous ammonium carbonate/acetonitrile) to afford Example 247 (18.3 mg, 23%) as a white solid. ¹H NMR (400 MHZ, DMSO-d₆) δ 11.89 (br. s, 1H), 10.57 (s, 1H), 8.33-7.89 (m, 2H), 7.45 (s, 1H), 5.93 (s, 2H), 5.77 (s, 1H), 4.75-4.56 (m, 1H), 3.97-3.90 (m, 1H), 3.89-3.83 (m, 3H), 3.75 (s, 6H), 3.70 (d, J=5.38 Hz, 2H), 3.63-3.52 (m, 1H), 2.89-2.73 (m, 3H), 2.28-2.14 (m, 1H), 1.90-1.70 (m, 2H), 0.98-0.86 (m, 2H), 0.71-0.60 (m, 2H); LCMS m/z (ESI+) for C₂₇H₃₂N₆O₇S 585.2 (M+H)⁺.

The Examples in Table 25 were prepared in a similar fashion to Example 247

TABLE 25

| Example | | |
|---|---|---|
| Number | Structure/IUPAC Name | Analytical Data |
| 248 | 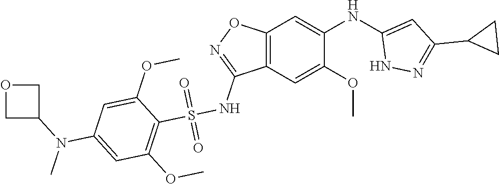 [SMILES: COc1cc2c(NS(=O)(=O)c3c(OC)cc(N(C)C4COC4)cc3OC)noc2cc1Nc1cc(C2CC2)n[nH]1] | ¹H NMR (400 MHz, DMSO-d₆) δ 11.93-11.87 (m, 1H), 10.66-10.58 (m, 1H), 8.15-8.08 (m, 1H), 8.07- 7.99 (m, 1H), 7.49-7.41 (m, 1H), 5.87-5.71 (m, 3H), 5.02-4.93 (m, 1H), 4.80-4.73 (m, 2H), 4.65-4.58 (m, 2H), 3.88-3.85 (m, 3H), 3.77- 3.70 (m, 6H), 3.00-2.95 (m, 3H), 1.92-1.79 (m, 1H), 0.97-0.85 (m, 2H), 0.71-0.62 (m, 2H); LCMS m/z (ESI+) for C₂₆H₃₀N₆O₇S, 571.4 (M + H)⁺ |
| | N-{6-[(3-cyclopropyl-1H-pyrazol-5- | |
| | yl)amino]-5-methoxy-1,2- | |
| | benzoxazol-3-yl}-2,6-dimethoxy-4- | |
| | [methyl(oxetan-3- | |
| | yl)amino]benzene-1-sulfonamide | |
| 249 | 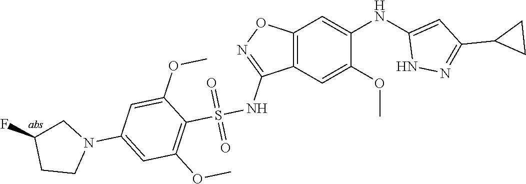 [SMILES: COc1cc2c(NS(=O)(=O)c3c(OC)cc(N4CC[C@@H](F)C4)cc3OC)noc2cc1Nc1cc(C2CC2)n[nH]1] | ¹H NMR (400 MHz, CD₃OD) δ 7.64 (br. s, 1H), 7.37 (s, 1H), 5.8-5.8 (m, 1H), 5.74 (s, 2H), 5.43 (br. s, 1H), 3.96 (s, 3H), 3.84 (s, 6H), 3.60 (br. d, J = 4.9 Hz, 1H,), 3.4-3.5 (m, 3H), 2.1-2.4 (m, 2H), 1.91 (s, 1H), 1.0- 1.0 (m, 2H), 0.7-0.8 (m, 2H); LCMS m/z (ESI+) for C₂₆H₂₉FN₆O₆S, 573.2 (M + H)⁺. |
| | N-{6-[(3-cyclopropyl-1H-pyrazol-5- | |
| | yl)amino]-5-methoxy-1,2- | |
| | benzoxazol-3-yl}-4-[(3R)-3- | |
| | fluoropyrrolidin-1-yl]-2,6- | |
| | dimethoxybenzene-1-sulfonamide | |
| 250 | 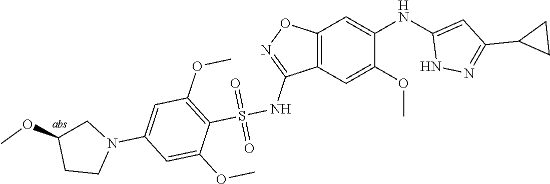 [SMILES: COc1cc2c(NS(=O)(=O)c3c(OC)cc(N4CC[C@@H](OC)C4)cc3OC)noc2cc1Nc1cc(C2CC2)n[nH]1] | ¹H NMR (400 MHz, CD₃OD) δ 7.6- 7.7 (m, 1H), 7.3-7.4 (m, 1H), 5.77 (s, 1H), 5.7-5.7 (m, 2H), 4.1-4.1 (m, 1H), 3.96 (s, 3H), 3.83 (s, 6H), 3.4- 3.5 (m, 1H), 3.3-3.4 (m, 6H), 2.0- 2.2 (m, 2H), 1.9-2.0 (m, 1H), 1.0- 1.0 (m, 2H), 0.7-0.8 (m, 2H); LCMS m/z (ESI+) for C₂₇H₃₂N₆O₇S, 585.1 (M + H)⁺. |
| | N-{6-[(3-cyclopropyl-1H-pyrazol-5- | |
| | yl)amino]-5-methoxy-1,2- | |
| | benzoxazol-3-yl}-2,6-dimethoxy-4- | |
| | [(3R)-3-methoxypyrrolidin-1- | |
| | yl]benzene-1-sulfonamide | |
| 251 | 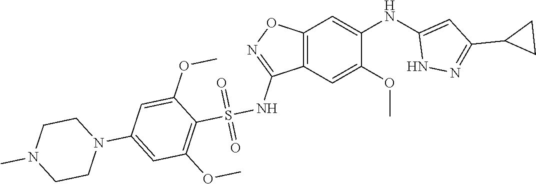 [SMILES: COc1cc2c(NS(=O)(=O)c3c(OC)cc(N4CCN(C)CC4)cc3OC)noc2cc1Nc1cc(C2CC2)n[nH]1] | ¹H NMR (400 MHz, DMSO-d₆) δ 11.95-11.85 (m, 1H), 10.84-10.42 (m, 1H), 8.13-8.10 (m, 1H), 8.04 (s, 1H), 7.44 (s, 1H), 6.08 (s, 2H), 5.78 (s, 1H), 3.87 (s, 3H), 3.74 (s, 6H), 3.39 (br. d, J = 3.5 Hz, 2H), 3.30- 3.28 (m, 2H), 2.41-2.35 (m, 4H), 2.20 (s, 3H), 1.89-1.83 (m, 1H), 0.94-0.89 (m, 2H), 0.68-0.64 (m, 2H); LCMS m/z (ESI+) for C₂₇H₃₃N₇O₆S, 584.3 (M + H)⁺ |
| | N-{6-[(3-cyclopropyl-1H-pyrazol-5- | |
| | yl)amino]-5-methoxy-1,2- | |
| | benzoxazol-3-yl}-2,6-dimethoxy-4- | |
| | (4-methylpiperazin-1-yl)benzene-1- | |
| | sulfonamide | |
| 252 | 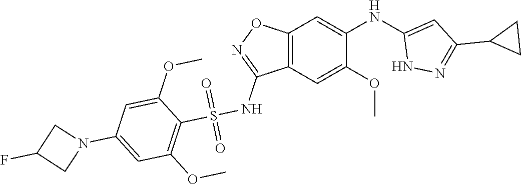 [SMILES: COc1cc2c(NS(=O)(=O)c3c(OC)cc(N4CC(F)C4)cc3OC)noc2cc1Nc1cc(C2CC2)n[nH]1] | ¹H NMR (400 MHz, DMSO-d₆) δ 12.00-11.80 (m, 1H), 10.78-10.29 (m, 1H), 8.04 (s, 2H), 7.50-7.38 (m, 1H), 5.82-5.74 (m, 1H), 5.66-5.57 (m, 2H), 5.57-5.36 (m, 1H), 4.30- 4.15 (m, 2H), 4.05-3.91 (m, 2H), 3.90-3.81 (m, 3H), 3.77-3.67 (m, 6H), 1.90-1.78 (m, 1H), 0.99-0.82 (m, 2H), 0.70-0.54 (m, 2H); LCMS m/z (ESI+) for C₂₅H₂₇FN₇O₆S, 559.2 (M + H)⁺ . |
| | N-{6-[(3-cyclopropyl-1H-pyrazol-5- | |
| | yl)amino]-5-methoxy-1,2- | |
| | benzoxazol-3-yl}-4-(3- | |
| | fluoroazetidin-1-yl)-2,6- | |
| | dimethoxybenzene-1-sulfonamide | |
| 253 | 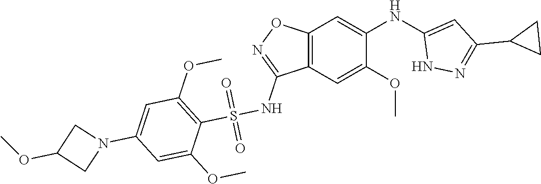 [SMILES: COc1cc2c(NS(=O)(=O)c3c(OC)cc(N4CC(OC)C4)cc3OC)noc2cc1Nc1cc(C2CC2)n[nH]1] | ¹H NMR (400 MHz, CD₃OD) δ 7.72-7.58 (m, 1H), 7.39-7.31 (m, 1H), 5.81-5.75 (m, 1H), 5.61-5.53 (m, 2H), 4.38-4.27 (m, 1H), 4.18- 4.08 (m, 1H), 4.11-4.08 (m, 1H), 3.99-3.93 (m, 3H), 3.86-3.78 (m, 6H), 3.79-3.72 (m, 2H), 3.32-3.32 (m, 3H), 1.97-1.86 (m, 1H), 1.06- 0.93 (m, 2H), 0.83-0.70 (m, 2H); LCMS m/z (ESI+) for C₂₆H₃₀N₆O₇S, 571.2 (M + H)⁺. |
| | N-{6-[(3-cyclopropyl-1H-pyrazol-5- | |
| | yl)amino]-5-methoxy-1,2- | |
| | benzoxazol-3-yl}-2,6-dimethoxy-4- | |
| | (3-methoxyazetidin-1-yl)benzene- | |
| | 1-sulfonamide | |
| 254 | 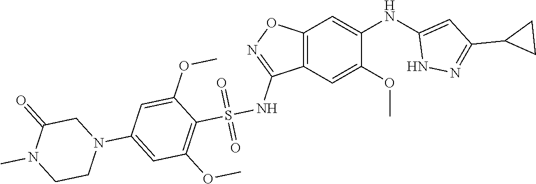 [SMILES: COc1cc2c(NS(=O)(=O)c3c(OC)cc(N4CCN(C)C(=O)C4)cc3OC)noc2cc1Nc1cc(C2CC2)n[nH]1] | ¹H NMR (400 MHz, DMSO-d₆) δ 12.08-11.70 (m, 1H), 10.81-10.41 (m, 1H), 8.22-7.91 (m, 2H), 7.44 (s, 1H), 6.04 (s, 2H), 5.83-5.68 (m, 1H), 3.95-3.89 (m, 2H), 3.89-3.83 (m, 3H), 3.80-3.72 (m, 6H), 3.64- 3.57 (m, 2H), 3.43-3.38 (m, 2H), 2.99-2.83 (m, 3H), 1.94-1.74 (m, 1H), 0.98-0.68 (m, 2H), 0.72-0.58 (m, 2H); LCMS m/z (ESI+) for C₂₇H₃₁N₇O₇S, 598.2 (M + H)⁺ |
| | N-{6-[(3-cyclopropyl-1H-pyrazol-5- | |
| | yl)amino]-5-methoxy-1,2- | |
| | benzoxazol-3-yl}-2,6-dimethoxy-4- | |
| | (4-methyl-3-oxopiperazin-1- | |
| | yl)benzene-1-sulfonamide | |

### Example 255: N-{6-[(3-cyclopropyl-1H-pyrazol-5-yl)amino]-5-methoxy-1,2-benzoxazol-3-yl}-2,6-dimethoxy-4-(6-oxo-5-azaspiro[3.4]octan-5-yl)benzene-1-sulfonamide

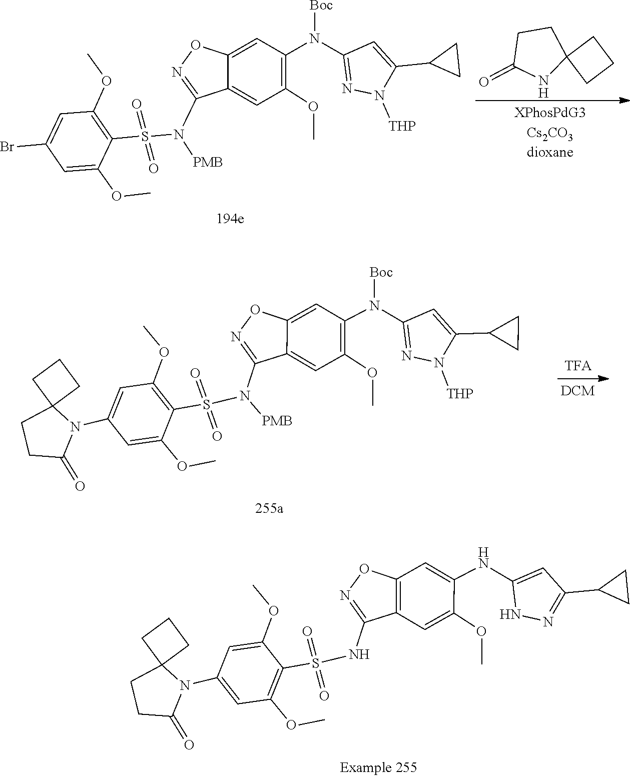

### Step 1: Synthesis of tert-butyl [5-cyclopropyl-1-(oxan-2-yl)-1H-pyrazol-3-yl](3-{[2,6-dimethoxy-4-(6-oxo-5-azaspiro[3.4]octan-5-yl)benzene-1-sulfonyl][(4-methoxyphenyl)methyl]amino}-5-methoxy-1,2-benzoxazol-6-yl) carbamate (255a)

A mixture of tert-butyl (3-{(4-bromo-2,6-dimethoxybenzene-1-sulfonyl) [(4-methoxyphenyl)methyl]amino}-5-methoxy-1,2-benzoxazol-6-yl) [5-cyclopropyl-1-(oxan-2-yl)-1H-pyrazol-3-yl]carbamate (194e) (180 mg, 207 mmol), 5-azaspiro[3.4]octan-6-one (64.8 mg, 0.518 mmol), and cesium carbonate (135 mg, 0.414 mmol) in 1,4-dioxane at rt was degassed via nitrogen sparge. To the mixture was added Xphos Pd G3 (26.3 mg, 0.0311 mmol). The mixture was heated to 95° C. and stirred for 16 hrs, and was then cooled and combined with that from another preparation run on 0.173 mmol. The combined lots were concentrated to afford 255a (500 mg, >99%) in crude form, which was taken into the next step without purification. LCMS m/z (ESI+) for C₄₇H₅₆N₆O₁₁S, 913.4 (M+H)⁺.

### Step 2. Synthesis of N-{6-[(3-cyclopropyl-1H-pyrazol-5-yl)amino]-5-methoxy-1,2-benzoxazol-3-yl}-2,6-dimethoxy-4-(6-oxo-5-azaspiro[3.4]octan-5-yl)benzene-1-sulfonamide (Example 255)

To a solution of tert-butyl [5-cyclopropyl-1-(oxan-2-yl)-1H-pyrazol-3-yl](3-{[2,6-dimethoxy-4-(6-oxo-5-azaspiro[3.4]octan-5-yl)benzene-1-sulfonyl][(4-methoxyphenyl)methyl]amino}-5-methoxy-1,2-benzoxazol-6-yl) carbamate 255a (345 mg, 0.270 mmol) in dichloromethane (2.0 mL) at rt was added trifluoroacetic acid (2.0 mL), and the resultant mixture was stirred for 16 hrs. The mixture was then concentrated and partitioned between water (10 mL) and dichloromethane (3×20 mL). The combined organic extracts were washed with brine, dried over sodium sulfate, and concentrated. The residue was purified via reversed-phase HPLC (Phenomenex Gemini NX 150×30 mm, 5 mm column, aqueous ammonium carbonate/acetonitrile) in two batches to afford Example 255 (11.7 mg, 7%) as a white solid. ¹H NMR (400 MHZ, DMSO-d₆) δ 11.91 (s, 1H) 11.10 (br. s, 1H) 8.14 (s, 1H) 8.07 (s, 1H) 7.38 (s, 1H) 6.51 (s, 2H) 5.78 (d, J=1.71 Hz, 1H) 3.87 (s, 3H) 3.76-3.73 (m, 6H) 2.42-2.37 (m, 2H) 2.31-2.24 (m, 2H) 2.23-2.10 (m, 2H) 2.02-1.90 (m, 2H) 1.89-1.81 (m, 1H) 1.68-1.57 (m, 1H) 1.47 (br. d, J=10.15 Hz, 1H) 0.95-0.87 (m, 2H) 0.68-0.63 (m, 2H); LCMS m/z for C₂₉H₃₂N₆O₇S, 609.2 (M+H)⁺.

### Example 256: N-{6-[(3-cyclopropyl-1H-pyrazol-5-yl)amino]-5-methoxy-1,2-benzoxazol-3-yl}-4-(2,2-dimethyl-5-oxopyrrolidin-1-yl)-2,6-dimethoxybenzene-1-sulfonamide

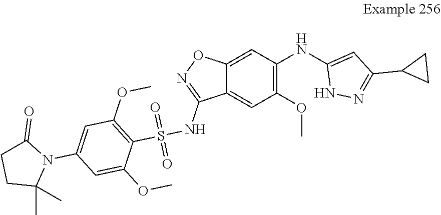 [SMILES: COc1cc2c(NS(=O)(=O)c3c(OC)cc(N4C(=O)CCC4(C)C)cc3OC)noc2cc1Nc1cc(C2CC2)n[nH]1]

Example 256 was prepared in a similar fashion to Example 255. ¹H NMR (400 MHZ, DMSO-d₆) 11.90 (br. s, 1H), 11.43-10.65 (m, 1H), 8.24-7.90 (m, 2H), 7.41-7.29 (m, 1H), 6.60-6.43 (m, 2H), 5.86-5.71 (m, 1H), 3.87 (s, 3H), 3.76-3.69 (m, 6H), 2.47-2.40 (m, 2H), 2.04-1.91 (m, 2H), 1.90-1.77 (m, 1H), 1.31-1.12 (m, 6H), 0.98-0.84 (m, 2H), 0.70-0.59 (m, 2H); LCMS m/z (ESI+) for C₂₈H₃₂N₆O₇S, 597.2 (M+H)⁺.

### Example 257: N-{6-[(3-cyclopropyl-1H-pyrazol-5-yl)amino]-5-methoxy-1,2-benzoxazol-3-yl}-4-(3,3-difluoropyrrolidine-1-carbonyl)-2,6-dimethoxybenzene-1-sulfonamide

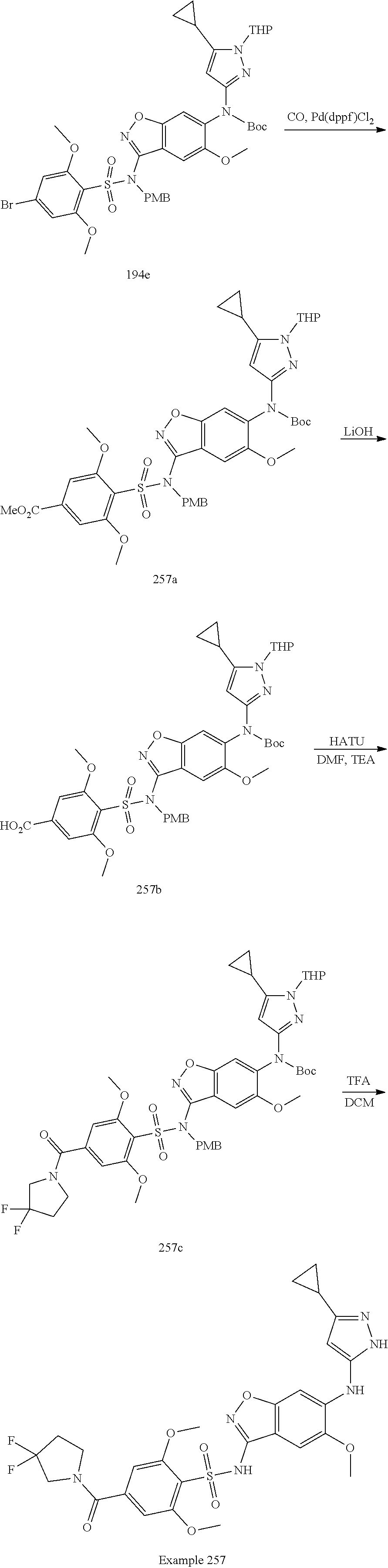

### Step 1: Synthesis of methyl 4-{(6-{(tert-butoxycarbonyl) [5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxybenzoate (257a)

A mixture of tert-butyl (3-{(4-bromo-2,6-dimethoxybenzene-1-sulfonyl) [(4-methoxyphenyl)methyl]amino}-5-hydroxy-1,2-benzoxazol-6-yl) [5-cyclopropyl-1-(oxan-2-yl)-1H-pyrazol-3-yl]carbamate 194e (3.00 g, 3.45 mmol) and Pd(dppf)Cl₂ (505 mg, 0.691 mmol) in methanol (173 mL) was degassed twice via vacuum/nitrogen purge, and the nitrogen was then replaced with a 50 psi atmosphere of carbon monoxide. The mixture was heated to 65° C. and stirred for 16 hrs, and then was cooled and the pressure released. The mixture was concentrated, and the residue was purified by flash column chromatography (ethyl acetate/petroleum ether) to afford 257a (2.92 g, 99%) as a purple solid which was progressed directly to step 2 without further purification.

### Step 2: Synthesis of 4-{(6-{(tert-butoxycarbonyl) [5-cyclopropyl-1-(oxan-2-yl)-1H-pyrazol-3-yl]amino}-5-methoxy-1,2-benzoxazol-3-yl)[(4-methoxyphenyl)methyl]sulfamoyl]-3,5-dimethoxybenzoic acid (257b)

To a solution of methyl 4-{(6-{(tert-butoxycarbonyl) [5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxybenzoate 257a (2.92 g, 3.44 mmol) in THF at rt was added a solution of lithium hydroxide hydrate (723 mg, 17.2 mmol) in water (6.88 mL). After stirring for 16 hrs, the mixture was diluted with water (10 mL) and the aqueous phase was adjusted to PH ˜6 with 1 M HCl. The resultant precipitate was collected via filtration, rinsed with water (20 mL×3) and dried to 257b (2.8 g, 97%) as a brown solid. ¹H NMR (400 MHZ, DMSO-d₆) δ 14.17-12.50 (m, 1H), 7.53 (s, 2H), 7.31 (br. d, J=8.3 Hz, 2H), 7.24 (s, 2H), 7.14-6.99 (m, 1H), 6.84 (br. d, J=8.4 Hz, 2H), 6.11-5.94 (m, 1H), 5.37 (br. d, J=8.8 Hz, 1H), 5.09-4.92 (m, 2H), 3.77 (s, 6H), 3.66 (br. d, J=12.4 Hz, 6H), 2.01-1.78 (m, 4H), 1.69-1.60 (m, 1H), 1.57-1.48 (m, 1H), 1.45-1.36 (m, 2H), 1.27 (br. s, 9H), 1.00-0.86 (m, 2H), 0.74-0.53 (m, 2H); LCMS m/z (ESI+) for C₄₁H₄₇N₅O₁₂S, 834.2 (M+H)⁺.

### Step 3: Synthesis of tert-butyl [5-cyclopropyl-1-(oxan-2-yl)-1H-pyrazol-3-yl](3-{[4-(3,3-difluoropyrrolidine-1-carbonyl)-2,6-dimethoxybenzene-1-sulfonyl][(4-methoxyphenyl)methyl]amino}-5-methoxy-1,2-benzoxazol-6-yl) carbamate (257c)

To a solution of 4-{(6-{(tert-butoxycarbonyl)[5-cyclopropyl-1-(oxan-2-yl)-1H-pyrazol-3-yl]amino}-5-methoxy-1,2-benzoxazol-3-yl)[(4-methoxyphenyl)methyl]sulfamoyl}-3,5-dimethoxybenzoic acid 257b (1500 mg, 0.179 mmol) in DMF (1.00 mL) at rt was added triethylamine (72.8 mg, 0.720 mmol), HATU 82 mg, 0.216 mmol), and 3,3-difluoropyrrolidine hydrochloride (31 mg, 0.216 mmol). The mixture was stirred for 18 hrs and was then poured into saturated aqueous sodium carbonate (8.0 mL). The mixture was extracted with ethyl acetate (3×10 mL) and the combined extracts were then dried over sodium sulfate and concentrated to afford 257c (130 mg, 78%) as a brown solid which was taken directly into the next step. LCMS m/z (ESI+) for C₄₅H₅₂F₂N₆O₁₁S, 923.2 (M+H)⁺.

### Step 4: Synthesis of N-{6-[(3-cyclopropyl-1H-pyrazol-5-yl)amino]-5-methoxy-1,2-benzoxazol-3-yl}-4-(3,3-difluoropyrrolidine-1-carbonyl)-2,6-dimethoxybenzene-1-sulfonamide (Example 257)

A solution of tert-butyl [5-cyclopropyl-1-(oxan-2-yl)-1H-pyrazol-3-yl](3-{[4-(3,3-difluoropyrrolidine-1-carbonyl)-2,6-dimethoxybenzene-1-sulfonyl][(4-methoxyphenyl)methyl]amino}-5-methoxy-1,2-benzoxazol-6-yl) carbamate 257c (130.0 mg, 0.141 mmol) in dichloromethane (3.0 mL) and trifluoroacetic acid (3.0 mL) at rt was stirred for 16 hrs. The mixture was concentrated and purified by HPLC (C18 column, aqueous ammonium carbonate/acetonitrile) to Example 257 (34.8 mg, 40%) as a white solid. ¹H NMR (400 MHZ, DMSO-d₆) δ 12.0-11.9 (m, 1H), 11.2-11.0 (m, 1H), 8.2-8.0 (m, 2H), 7.4-7.3 (m, 1H), 6.9-6.7 (m, 2H), 5.8-5.5 (m, 1H), 3.9-3.8 (m, 5H), 3.8-3.8 (m, 6H), 3.68 (br. t, J=7.5 Hz, 1H), 3.60 (br. t, J=7.3 Hz, 1H), 2.5-2.3 (m, 2H), 1.9-1.8 (m, 1H), 1.0-0.8 (m, 2H), 0.7-0.6 (m, 2H); LCMS m/z (ESI+) for C₂₇H₂₈F₂N₆O₇S, 619.2 (M+H)⁺.

The Examples in Table 26 were prepared similarly to Example 257 using commercially available amines for the amide coupling (see step 3).

TABLE 26

| Example | | |
|---|---|---|
| Number | Structure/IUPAC Name | Analytical Data |
| 258 | 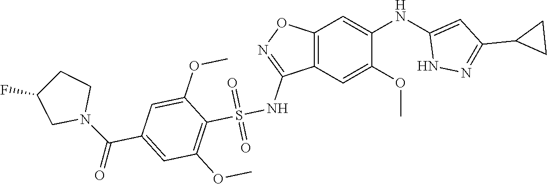 [SMILES: COc1cc2c(NS(=O)(=O)c3c(OC)cc(C(=O)N4CC[C@@H](F)C4)cc3OC)noc2cc1Nc1cc(C2CC2)n[nH]1] | LCMS m/z (ESI+) for C₂₇H₂₉FN₆O₇S, 601.2 [M + H]⁺. ¹H NMR (400 MHz, DMSO-d₆) δ 11.96-11.85 (m, 1H) 11.24-10.95 (m, 1H) 8.20-8.10 (m, 1 H) 8.06 (s, 1H) 7.37 (s, 1H) 6.81 (d, J = 5.4 Hz, 2H) 5.78 (s, 1H) 5.46-5.19 (m, 1H) 3.87 (s, 3H) 3.78 (d, J = 2.45 Hz, 6H) 3.73-3.62 (m, 2H) 3.59- 3.48 (m, 1H) 3.48-3.40 (m, 1H) 2.21-1.94(m, 2H) 1.89-1.81 (m, 1H) 0.97-0.86 (m, 2H) 0.69-0.63 (m, 2H). |
| | N-{6-[(3-cyclopropyl-1H-pyrazol-5- | |
| | yl)amino]-5-methoxy-1,2-benzoxazol-3- | |
| | yl}-4-[(3R)-3-fluoropyrrolidine-1- | |
| | carbonyl]-2,6-dimethoxybenzene-1- | |
| | sulfonamide | |
| 259 | 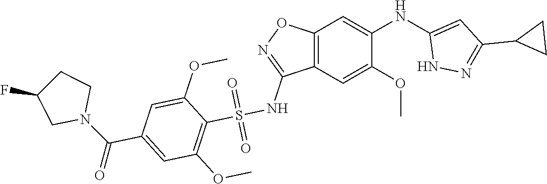 [SMILES: COc1cc2c(NS(=O)(=O)c3c(OC)cc(C(=O)N4CC[C@H](F)C4)cc3OC)noc2cc1Nc1cc(C2CC2)n[nH]1] | LCMS m/z (ESI+) for C₂₇H₂₉FN₆O₇S, 601.2 [M + H]⁺. ¹H NMR (400 MHz, DMSO-d₆) δ 11.91 (s, 1H), 11.38-10.83 (m, 1H), 8.29-7.91 (m, 2H), 7.54-7.23 (m, 1H), 6.94-6.67 (m, 2H), 5.78 (s, 1H), 5.49-5.09 (m, 1H), 3.93-3.84 (m, 3H), 3.79 (d, J = 2.63 Hz, 6H), 3.75-3.69 (m, 1H), 3.69-3.61 (m, 1 H), 3.59 (br. d, J = 7.00 Hz, 1H), 3.47-3.41 (m, 1 H), 2.22-1.96 (m, 2H), 1.92-1.79 (m, 1H), 0.85 (br. s, 2H), 0.73-0.60 (m, 2H). |
| | N-{6-[(3-cyclopropyl-1H-pyrazol-5- | |
| | yl)amino]-5-methoxy-1,2-benzoxazol-3- | |
| | yl}-4-[(3S)-3-fluoropyrrolidine-1- | |
| | carbonyl]-2,6-dimethoxybenzene-1- | |
| | sulfonamide | |
| 260 | 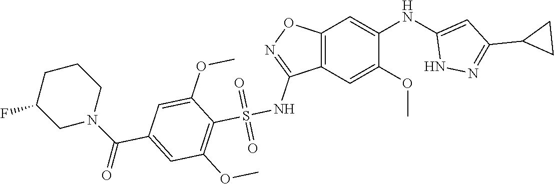 [SMILES: COc1cc2c(NS(=O)(=O)c3c(OC)cc(C(=O)N4CCC[C@@H](F)C4)cc3OC)noc2cc1Nc1cc(C2CC2)n[nH]1] | LCMS m/z (ESI+) for C₂₈H₃₁FN₆O₇S, 615.2 [M + H]⁺. ¹H NMR (400 MHz, DMSO-d₆) δ 12.0-11.8(m, 1H), 11.3-10.9(m, 1H), 8.13 (s, 1H), 8.1-8.0 (m, 1H), 7.4-7.3 (m, 1H), 6.7-6.6 (m, 2H), 5.78 (s, 1H), 4.9-4.6 (m, 1H), 4.01 (br. s, 1H), 3.87 (s, 3H), 3.76 (br. s, 6H), 3.6-3.5 (m, 1H), 3.3-3.2 (m, 1H), 3.2-3.0 (m, 1H), 1.9-1.4 (m, 5H), 0.9-0.9 (m, 2H), 0.7-0.6 (m, 2H). |
| | N-{6-[(3-cyclopropyl-1H-pyrazol-5- | |
| | yl)amino]-5-methoxy-1,2-benzoxazol-3- | |
| | yl}-4-[(3R)-3-fluoropiperidine-1-carbonyl]- | |
| | 2,6-dimethoxybenzene-1-sulfonamide | |
| 261 | 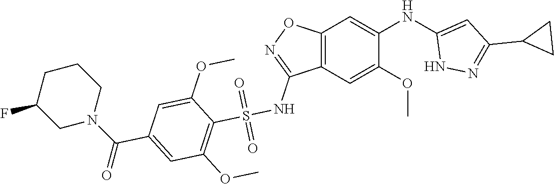 [SMILES: COc1cc2c(NS(=O)(=O)c3c(OC)cc(C(=O)N4CCC[C@H](F)C4)cc3OC)noc2cc1Nc1cc(C2CC2)n[nH]1] | LCMS m/z (ESI+) for C₂₈H₃₁FN₆O₇S, 615.1 [M + H]⁺. ¹H NMR (400 MHz, DMSO-d₆) δ 12.05-11.87 (m, 1H), 11.25-11.02 (m, 1H), 8.23-7.94 (m, 2H), 7.47- 7.24 (m, 1H), 6.81-6.56 (m, 2H), 5.84-5.70 (m, 1H), 5.00-4.61 (m, 1H), 4.21-3.98 (m, 1H), 3.94-3.83 (m, 3H), 3.81-3.71 (m, 6H), 3.63- 3.47 (m, 1H), 3.31-3.23 (m, 1H), 3.22-2.95 (m, 1H), 1.96-1.73 (m, 3H), 1.72-1.39 (m, 2H), 0.98-0.86 (m, 2H), 0.71-0.61 (m, 2H). |
| | N-{6-[(3-cyclopropyl-1H-pyrazol-5- | |
| | yl)amino]-5-methoxy-1,2-benzoxazol-3- | |
| | yl}-4-[(3S)-3-fluoropiperidine-1-carbonyl]- | |
| | 2,6-dimethoxybenzene-1-sulfonamide | |
| 262 | 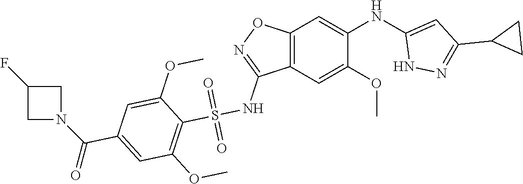 [SMILES: COc1cc2c(NS(=O)(=O)c3c(OC)cc(C(=O)N4CC(F)C4)cc3OC)noc2cc1Nc1cc(C2CC2)n[nH]1] | LCMS m/z (ESI+) for C₂₆H₂₇FN₆O₇S, 587.1 [M + H]⁺. ¹H NMR (400 MHz, DMSO-d₆) δ 11.91 (br. s, 1H), 11.28-11.01 (m, 1H), 8.26-7.96 (m, 2H), 7.50-7.25 (m, 1H), 7.02-6.68 (m, 2H), 5.83- 5.69 (m, 1H), 5.56-5.23 (m, 1H), 4.61-4.28 (m, 3H), 4.16-4.00 (m, 1H), 3.95-3.86 (m, 3H), 3.86-3.69 (m, 6H), 2.00-1.76 (m, 1 H), 1.01- 0.83 (m, 2H), 0.78-0.57 (m, 2H). |
| | N-{6-[(3-cyclopropyl-1H-pyrazol-5- | |
| | yl)amino]-5-methoxy-1,2-benzoxazol-3- | |
| | yl}-4-(3-fluoroazetidine-1-carbonyl)-2,6- | |
| | dimethoxybenzene-1-sulfonamide | |
| 263 | 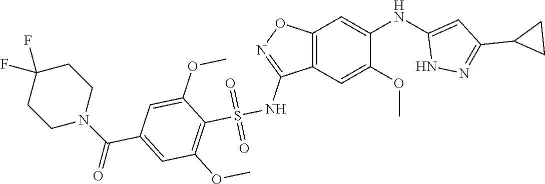 [SMILES: COc1cc2c(NS(=O)(=O)c3c(OC)cc(C(=O)N4CCC(F)(F)CC4)cc3OC)noc2cc1Nc1cc(C2CC2)n[nH]1] | LCMS m/z (ESI+) for C₂₈H₃₀F₂N₆O₇S, 633.2 [M + H]⁺. ¹H NMR (400 MHz, DMSO-d₆) δ 12.03-11.89 (m, 1H), 11.23-10.94 (m, 1H), 8.18-7.98 (m, 2H), 7.47- 7.29 (m, 1H), 6.87-6.68 (m, 2H), 5.94-5.66 (m, 1H), 3.92-3.83 (m, 3H), 3.81-3.72 (m, 6H), 3.72-3.57 (m, 2H), 3.31-3.26 (m, 2H), 2.15- 1.93 (m, 4H), 1.89-1.79 (m, 1H), 1.05-0.84 (m, 2H), 0.72-0.57 (m, 2H). |
| | N-{6-[(3-cyclopropyl-1H-pyrazol-5- | |
| | yl)amino]-5-methoxy-1,2-benzoxazol-3- | |
| | yl}-4-(4,4-difluoropiperidine-1-carbonyl)- | |
| | 2,6-dimethoxybenzene-1-sulfonamide | |
| 264 | 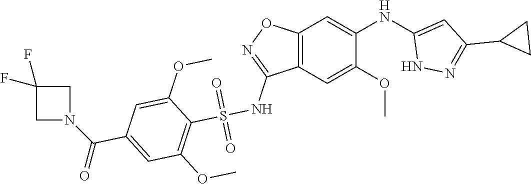 [SMILES: COc1cc2c(NS(=O)(=O)c3c(OC)cc(C(=O)N4CC(F)(F)C4)cc3OC)noc2cc1Nc1cc(C2CC2)n[nH]1] | LCMS m/z (ESI+) for C₂₆H₂₆F₂N₆O₇S, 605.1 [M + H]⁺. ¹H NMR (400 MHz, DMSO-d₆) δ ppm 11.90 (br. s, 1H), 11.01- 11.24 (m, 1H), 8.14 (s, 1H), 8.07 (s, 1H), 7.37 (s, 1H), 6.91 (s, 2H), 5.78 (s, 1H), 4.80 (br. t, J = 11.07 Hz, 2H), 4.46 (br. t, J = 11.80 Hz, 2H), 3.88 (s, 3H), 3.81 (s, 6H), 1.80- 1.90 (m, 1H), 0.87-0.95 (m, 2H), 0.62-0.69 (m, 2H |
| | N-{6-[(3-cyclopropyl-1H-pyrazol-5- | |
| | yl)amino]-5-methoxy-1,2-benzoxazol-3- | |
| | yl}-4-(3,3-difluoroazetidine-1-carbonyl)- | |
| | 2,6-dimethoxybenzene-1-sulfonamide | |

### Example 265: N-{6-[(3-cyclopropyl-1H-pyrazol-5-yl)amino]-5-methoxy-1,2-benzoxazol-3-yl}-2,6-dimethoxy-4-(oxan-4-yl) benzene-1-sulfonamide

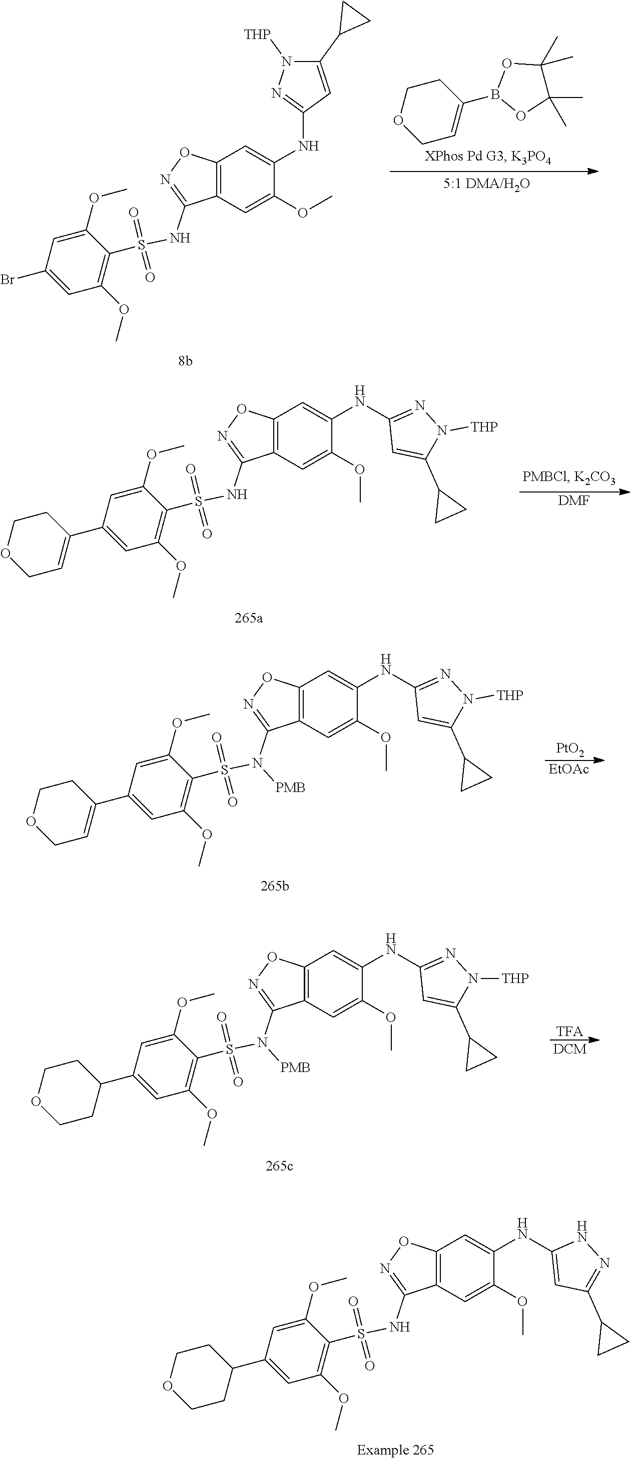

### Step 1: Synthesis of N-(6-{[5-cyclopropyl-1-(oxan-2-yl)-1H-pyrazol-3-yl]amino}-5-methoxy-1,2-benzoxazol-3-yl)-4-(3,6-dihydro-2H-pyran-4-yl)-2,6-dimethoxybenzene-1-sulfonamide (265a)

To a solution of 4-bromo-N-(6-{[5-cyclopropyl-1-(oxan-2-yl)-1H-pyrazol-3-yl]amino}-5-methoxy-1,2-benzoxazol-3-yl)-2,6-dimethoxybenzene-1-sulfonamide (8b) (250 mg, 0.386 mmol) and 2-10 (3,6-dihydro-2H-pyran-4-yl)-4,4,5,5-tetramethyl-1,3,2-dioxaborolane (121 mg, 0.578 mmol) in DMA (1.0 mL) and water (0.2 mL) was added tripotassium phosphate (245 mg, 1.16 mmol) and XPhos Pd G3 (32.6 mg, 0.385 mmol). The mixture was degassed 5 times with nitrogen and stirred at 80° C. for 3 hrs. The mixture was then diluted with water, extracted with EtOAc (3×12 mL), and washed with water (10 mL), and the combined organic layers were dried over Na₂SO₄ and concentrated under reduced pressure. The crude residue was purified by column chromatography (20 g silica gel, 0-95% EtOAc/Pet. ether) to afford 265a (160 mg, 64%) as a yellow solid. LCMS m/z 652.1 (M+H)⁺.

### Step 2: Synthesis of N-(6-{[5-cyclopropyl-1-(oxan-2-yl)-1H-pyrazol-3-yl]amino}-5-methoxy-1,2-benzoxazol-3-yl)-4-(3,6-dihydro-2H-pyran-4-yl)-2,6-dimethoxy-N-[(4-methoxyphenyl)methyl]benzene-1-sulfonamide (265b)

To a solution of N-(6-{[5-cyclopropyl-1-(oxan-2-yl)-1H-pyrazol-3-yl]amino}-5-methoxy-1,2-benzoxazol-3-yl)-4-(3,6-dihydro-2H-pyran-4-yl)-2,6-dimethoxybenzene-1-sulfonamide 265a (160 mg, 0.245 mmol) and potassium carbonate (136 mg, 0.982 mmol) in DMF (2.46 mL) was added para-methoxybenzyl chloride (46.1 mg, 0.295 mmol) dropwise. The resulting mixture was stirred at 80° C. for 2 hrs, then was diluted with water, extracted with EtOAc (3×12 mL) and washed with water (10 mL). The combined organic layers were dried over Na₂SO₄, filtered and concentrated under reduced pressure. The crude residue was purified by column chromatography (12 g silica gel, 0-70% EtOAc/Pet. ether) to afford 265b (110 mg, 58%) as a pale purple solid. LCMS m/z 772.2 (M+H)⁺.

### Step 3: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(oxan-4-yl)benzene-1-sulfonamide (265c)

To a solution of N-(6-{[5-cyclopropyl-1-(oxan-2-yl)-1H-pyrazol-3-yl]amino}-5-methoxy-1,2-benzoxazol-3-yl)-4-(3,6-dihydro-2H-pyran-4-yl)-2,6-dimethoxy-N-[(4-methoxyphenyl)methyl]benzene-1-sulfonamide 265b (110 mg, 0.1425 mmol) in EtOAc (1.43 mL) was added platinum dioxide (7.77 mg, 0.034 mmol) at rt. The solution was stirred at rt for 16 hrs under a hydrogen balloon. The resulting mixture was concentrated under reduced pressure and used directly in the next step (quant. yield assumed). LCMS m/z 774.4 (M+H)⁺.

### Step 4: Synthesis of N-{6-[(3-cyclopropyl-1H-pyrazol-5-yl)amino]-5-methoxy-1,2-benzoxazol-3-yl}-2,6-dimethoxy-4-(oxan-4-yl)benzene-1-sulfonamide (Example 265)

To a solution of N-(6-{[5-cyclopropyl-1-(oxan-2-yl)-1H-pyrazol-3-yl]amino}-5-methoxy-1,2-benzoxazol-3-yl)-2,6-dimethoxy-N-[(4-methoxyphenyl)methyl]-4-(oxan-4-yl)benzene-1-sulfonamide 265c (110 mg, 0.142 mmol) in DCM (1.0 mL) was added TFA (2.0 mL). The mixture was stirred at rt for 4 hrs, then concentrated under reduced pressure. The crude residue was purified by preparative HPLC (Phenomenex Gemini NX 150×30 mm column, 5 μm particle size, Mobile phase A: Water+ammonia hydroxide Mobile phase B: ACN 1-41% over 9 mins, flow rate 60 mL/mins) to give Example 265 (27.6, 34%) as a white solid. ¹H NMR (400 MHZ, DMSO-d₆) δ 11.90 (br. s, 1H), 10.91 (br. s, 1H), 8.12 (br. s, 1H), 8.05 (br. s, 1H), 7.41 (br. s, 1H), 6.63 (s, 2H), 5.78 (br. s, 1H), 3.93 (br. d, J=9.9 Hz, 2H), 3.87 (br. s, 3H), 3.76 (s, 6H), 3.44-3.35 (m, 2H), 2.85-2.71 (m, 1H), 1.93-1.80 (m, 1H), 1.78-1.60 (m, 4H), 1.00-0.87 (m, 2H), 0.70-0.59 (m, 2H); LCMS m/z 570.1 (M+H)⁺.

### Example 266 and 267: N-{6-[(3-cyclopropyl-1H-pyrazol-5-yl)amino]-5-methoxy-1,2-benzoxazol-3-yl}-2,6-dimethoxy-4-(oxolan-3-yl)benzene-1-sulfonamide

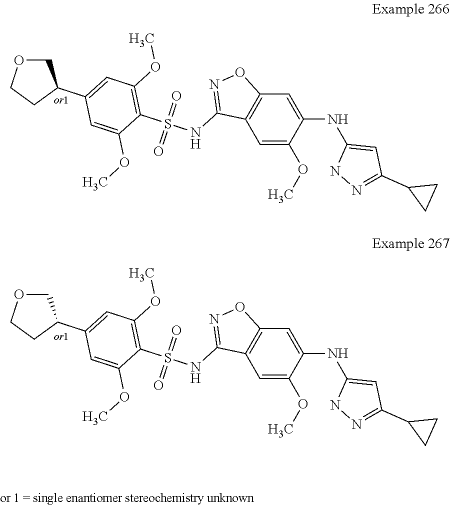

Examples 266 and 267 were prepared analogous to Examples 142 and 143 with 2-(4,5-dihydrofuran-3-yl)-4,4,5,5-tetramethyl-1,3,2-dioxaborolane as the coupling partner. The enantiomers were separated by chiral SFC (Daicel Chiralcel OJ (250×30 mm, 10 μm, eluting with CO₂/MeOH (modified with 0.1% ammonium hydroxide) at a flow rate of 80 mL/mins) to afford N-{6-[(3-cyclopropyl-1H-pyrazol-5-yl)amino]-5-methoxy-1,2-benzoxazol-3-yl}-2,6-dimethoxy-4-(oxolan-3-yl)benzene-1-sulfonamide (Example 266) (6.3 mg, 16% yield, >99% ee) as a white solid and the first eluting peak. ¹H NMR (400 MHZ, METHANOL-d₄) δ 7.68-7.58 (m, 1H), 7.41-7.25 (m, 1H), 6.69-6.58 (m, 2H), 5.82-5.71 (m, 1H), 4.09-4.01 (m, 2H), 3.99-3.94 (m, 3H), 3.88-3.82 (m, 7H), 3.78-3.70 (m, 1H), 3.47-3.38 (m, 1H), 2.43-2.32 (m, 1H), 2.06-1.85 (m, 2H), 1.04-0.96 (m, 2H), 0.80-0.71 (m, 2H); LCMS m/z 556.2 (M+H)⁺, rt=0.857.

N-{6-[(3-cyclopropyl-1H-pyrazol-5-yl)amino]-5-methoxy-1,2-benzoxazol-3-yl}-2,6-dimethoxy-4-(oxolan-3-yl)benzene-1-sulfonamide (Example 267) (4.5 mg, 11% yield, 94% ee) as a white solid and the second eluting peak. ¹H NMR (400 MHZ, METHANOL-d₄) δ 7.70-7.53 (m, 1H), 7.37-7.27 (m, 1H), 6.67-6.60 (m, 2H), 5.81-5.74 (m, 1H), 4.11-4.00 (m, 2H), 3.99-3.93 (m, 3H), 3.92-3.78 (m, 7H), 3.77-3.69 (m, 1H), 3.49-3.37 (m, 1H), 2.43-2.32 (m, 1H), 2.06-1.87 (m, 2H), 1.04-0.95 (m, 2H), 0.82-0.69 (m, 2H); LCMS m/z 556.2 (M+H)⁺, rt=0.857.

### Examples 268 and 269: N-{6-[(3-cyclopropyl-1H-pyrazol-5-yl)amino]-5-methoxy-1,2-benzoxazol-3-yl}-2,6-dimethoxy-4-(oxan-3-yl)benzene-1-sulfonamide

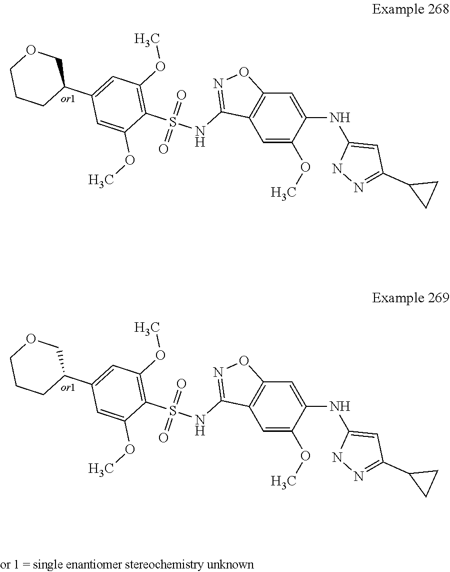

Examples 268 and 269 were prepared analogous to 142 and 143 with 5-(4,4,5,5-tetramethyl-1,3,2-dioxaborolan-2-yl)-3,4-dihydro-2H-pyran as the coupling partner. The enantiomers were separated by chiral SFC (Daicel Chiralcel OJ (250×30 mm, 10 μm, eluting with CO₂/EtOH (modified with 0.1% ammonium hydroxide) at a flow rate of 80 mL/mins) to afford N-{6-[(3-cyclopropyl-1H-pyrazol-5-yl)amino]-5-methoxy-1,2-benzoxazol-3-yl}-2,6-dimethoxy-4-(oxan-3-yl)benzene-1-sulfonamide (Example 268) (8.4 mg, 28% yield, >99% ee) as a white solid and the first eluting peak. ¹H NMR (400 MHZ, DMSO-d₆) δ 11.90 (br. s, 1H) 8.13 (s, 1H) 8.07 (s, 1H) 7.40 (s, 1H) 6.64 (s, 2H) 5.78 (s, 1H) 3.86 (s, 3H) 3.85-3.78 (m, 2H) 3.76 (s, 6H) 3.41-3.36 (m, 2H) 2.83-2.73 (m, 1H) 1.92-1.83 (m, 2H) 1.76 (br. dd, J=11.99, 4.73 Hz, 1H) 1.67-1.55 (m, 2H) 0.96-0.85 (m, 2H) 0.72-0.62 (m, 2H); LCMS m/z 570.3 (M+H)+, rt=0.909. [a],22=−6° (C. 1.0 mg/mL MeOH).

N-{6-[(3-cyclopropyl-1H-pyrazol-5-yl)amino]-5-methoxy-1,2-benzoxazol-3-yl}-2,6-dimethoxy-4-(oxan-3-yl)benzene-1-sulfonamide (Example 269) (6.7 mg, 22% yield, 98% ee) as a white solid and the second eluting peak. ¹H NMR (400 MHZ, DMSO-d₆) δ 11.91 (br. s, 1H) 8.14 (s, 1H) 8.08 (s, 1H) 7.41 (s, 1H) 6.64 (s, 2H) 5.79 (s, 1H) 3.87 (s, 3H) 3.85-3.79 (m, 2H) 3.77 (s, 6H) 3.40 (br. s, 2H) 2.78 (br. s, 1H) 1.90-1.84 (m, 2H) 1.76 (br. dd, J=11.66, 4.84 Hz, 1H) 1.65-1.59 (m, 2H) 0.94-0.88 (m, 2H) 0.68-0.63 (m, 2H); LCMS m/z 570.3 (M+H)+, rt=0.909. [a]_{D}²²=+1.3° (C. 1.0 mg/mL MeOH).

### Example 270: N-{6-[(3-cyclopropyl-1H-pyrazol-5-yl)amino]-5-methoxy-1,2-benzoxazol-3-yl}-2.6-dimethoxy-4-(methoxymethyl)benzene-1-sulfonamide

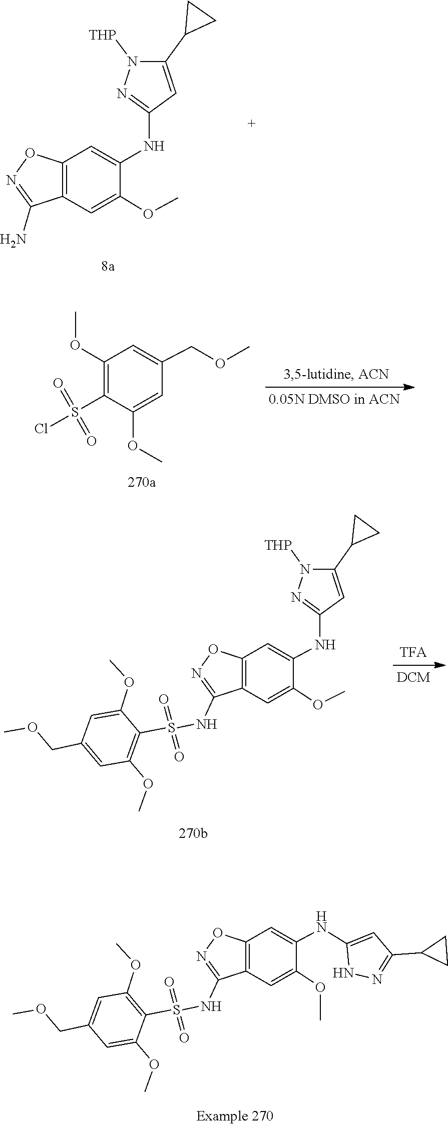

Step 1: Synthesis of 2,6-dimethoxy-4-(methoxymethyl)benzene-1-sulfonyl chloride (270a) 270a was prepared in a similar fashion as intermediate F starting with commercially available 1,3-dimethoxy-5-(methoxymethyl)benzene in place of 1-methoxy-3-(methoxymethoxy)benzene. ¹H NMR (400 MHZ, CDCl₃) d 6.67-6.61 (m, 2H), 4.52-4.47 (m, 2H), 4.03-3.98 (m, 6H), 3.51-3.44 (m, 3H).

### Step 2: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-(methoxymethyl)benzene-1-sulfonamide (270b)

To a solution of 8a (220 mg, 0.596 mmol) in ACN (1.2 mL) was added 0.05N DMSO in ACN (2 mg, 0.03 mmol) and 3,5-lutidine (204 mg, 1.91 mmol). 270a was added (251 mg 0.893 mmol) in six portions. The suspension was stirred at 60° C. for 16 hrs, then concentrated. The crude residue was purified by column chromatography (0-50% EtOAc/Pet. ether) to afford 270b (80 mg, 22%) as yellow oil. LCMS m/z 614.3 (M+H)⁺.

### Step 3: Synthesis of N-{6-[(3-cyclopropyl-1H-pyrazol-5-yl)amino]-5-methoxy-1,2-benzoxazol-3-yl}-2,6-dimethoxy-4-(methoxymethyl)benzene-1-sulfonamide (Example 176)

A solution 270b (80 mg, 0.13 mmol) in TFA (1.0 mL) was stirred at 25° C. for 3 hrs. The reaction was concentrated, and the residue was purified by preparative HPLC (C18 150×40 mm, Mobile phase A: Water+NH₃H₂O+NH₄HCO₃ Mobile phase B: ACN 5-45% over 9 mins, flow rate 60 mL/mins) to afford Example 270 (28.5 mg, 41%) as a white solid. ¹H NMR (400 MHZ, CDCl₃) δ 7.65 (s, 1H), 7.44 (s, 1H), 7.11 (br. s, 1H), 6.56 (s, 2H), 5.72 (s, 1H), 4.40 (s, 2H), 3.95 (s, 3H), 3.90 (s, 6H), 3.39 (s, 3H), 1.89-1.80 (m, 1H), 1.03-0.96 (m, 2H), 0.77-0.70 (m, 2H); LCMS m/z 530.3 (M+H)⁺.

### Example 271: N-{6-[(3-cyclopropyl-1H-pyrazol-5-yl)amino]-5-methoxy-1,2-benzoxazol-3-yl}-2,6-dimethoxy-4-(2-methoxypropan-2-yl)benzene-1-sulfonamide

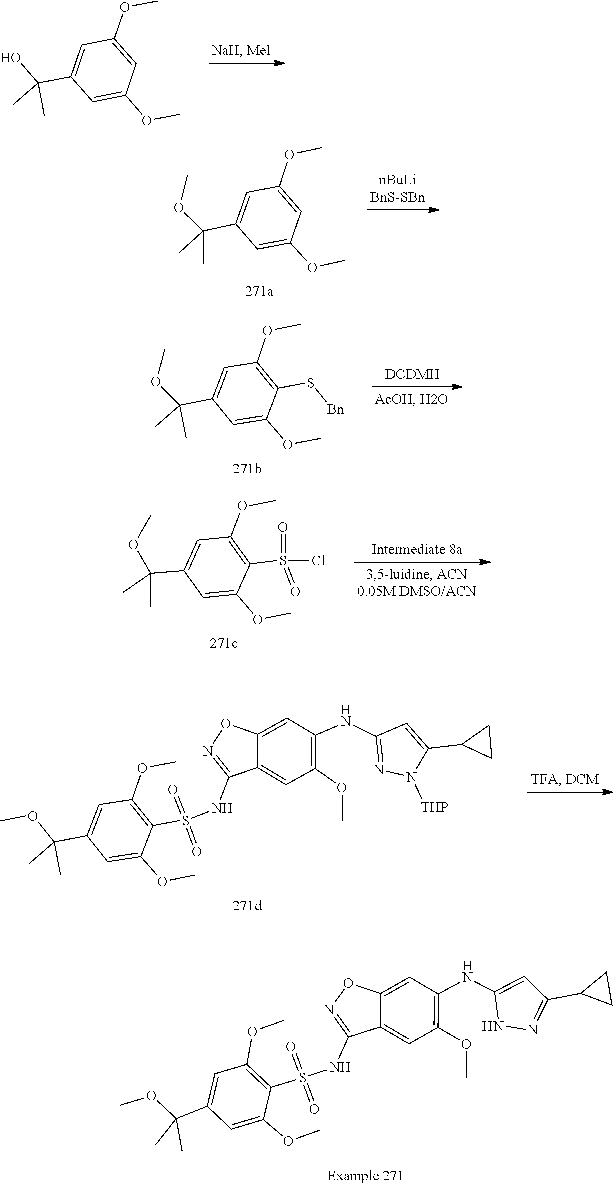

### Step 1: Synthesis of 1,3-dimethoxy-5-(2-methoxypropan-2-yl)benzene (271a)

To a stirred solution of 3,5-dimethoxy-α,α-dimethylbenzenemethanol (1.0 g, 5.1 mmol) in THF (17.0 mL) was added sodium hydride (306 mg, 7.64 mmol) at 5° C. and the mixture was allowed to stir for 20 mins. Methyl iodide (1.01 g, 7.13 mmol) was added and the resulting mixture was stirred at 30° C. for 16 hrs. The reaction was diluted with water (10 mL) and extracted with EtOAc (4×20 mL). The combined organic layers were washed with brine (20 mL), dried over Na₂SO₄, filtered, and concentrated under reduced pressure. The crude residue was purified by column chromatography (Silica gel, 0-30% EtOAc/Pet. ether) to afford 271a (0.87 g, 81%) as a white oil. ¹H NMR (400 MHZ, DMSO-d₆) d 6.50 (d, 2H, J=2.3 Hz), 6.40 (t, 1H, J=2.3 Hz), 3.74 (s, 6H), 2.98 (s, 3H), 1.42 (s, 6H); LCMS m/z 211.1 (M+H)⁺.

### Step 2: Synthesis of 2-(benzylsulfanyl)-1,3-dimethoxy-5-(2-methoxypropan-2-yl)benzene (271b)

To a stirred solution of 1,3-dimethoxy-5-(2-methoxypropan-2-yl)benzene 271a (630 mg, 3.0 mmol) in anhydrous THF (15.0 mL) was added dropwise n-butyllithium (240 mg, 3.75 mmol) at 5° C. under nitrogen. The mixture was stirred for 1 hr at 0° C., then dibenzyl disulfide was added under a nitrogen atmosphere in portions at 0° C. The resulting mixture was stirred at 0° C. for 2 hrs. The reaction was poured into water (20 mL) and extracted with EtOAc (2×20 mL). The organic layers were washed with water (2×20 ml), concentrated under reduced pressure, and dried over Na₂SO₄. The crude residue was purified by column chromatography (20 g silica gel, 0-30% EtOAc/Pet. ether) to afford 271b (840 mg, 84%) as a white gum. ¹H NMR (400 MHZ, CDCl₃) δ 7.21-7.12 (m, 5H), 6.57 (s, 2H), 3.97 (s, 2H), 3.80 (s, 6H), 3.09 (s, 3H), 1.52 (s, 6H); LCMS m/z 333.1 (M+H)⁺.

### Step 3:2,6-dimethoxy-4-(2-methoxypropan-2-yl)benzene-1-sulfonyl chloride (271c)

To a stirred solution of 2-(benzylsulfanyl)-1,3-dimethoxy-5-(2-methoxypropan-2-yl)benzene 271b (840 mg, 2.53 mmol) in AcOH (0.45 mL), water (0.3 mL) and ACN (8 mL) was added 1,3-dichloro-5,5-dimethylhydantoin (697 mg, 3.54 mmol) at 0° C. The mixture was stirred for 20 mins, then poured into water (10 mL) and extracted with EtOAc (2×10 mL). The combined organic layers were washed with brine (3×10 mL), dried over Na₂SO₄ and concentrated under reduced pressure. The crude residue was purified by column chromatography (12 g silica gel, 0-40% EtOAc/Pet. ether) to afford 271c (600 mg, 77%) as a white solid. ¹H NMR (400 MHZ, DMSO-d₆) d 6.71 (s, 2H), 4.01 (s, 6H), 3.17 (s, 3H), 1.55 (s, 6H).

### Step 4: Synthesis of 2,6-dimethoxy-4-(2-methoxypropan-2-yl)benzene-1-sulfonyl chloride (271d)

To a solution of 8a (200 mg, 0.541 mmol) and 271c (301 mg, 0.975 mmol) in ACN (2 mL) was added 0.05N DMSO in ACN (2.12 mg, 0.027 mmol) and 3,5-lutidine (174 mg, 1.62 mmol). The reaction was sealed and stirred at 60° C. for 16 hrs. The reaction mixture was poured into water (˜5 mL) and extracted with EtOAc (5 mL×2). The combined extracts were washed with brine (5 mL×3), dried over Na₂SO₄, filtered and concentrated. The crude residue was purified by column chromatography (12 g silica gel, 0-80% EtOAc/Pet. ether) to afford 271d (300 mg, 86%) as a white solid. LCMS m/z 642.3 (M+H)⁺.

### Step 5: Synthesis of N-{6-[(3-cyclopropyl-1H-pyrazol-5-yl)amino]-5-methoxy-1,2-benzoxazol-3-yl}-2,6-dimethoxy-4-(2-methoxypropan-2-yl)benzene-1-sulfonamide (Example 271)

A solution of 271d (300 mg, 0.467 mmol) in TFA (3 mL) and DCM (3 mL) was stirred at 25° C. for 16 hrs. The product was concentrated then purified by preparative HPLC (Xtimate C18 150×40 mm×5 mm Mobile phase A: Water+NH₃H₂O+NH₄HCO₃ Mobile phase B: ACN 8-48% over 9 mins, flow rate 60 mL/mins) to afford Example 271 (84.4 mg, 32%) as a white solid. ¹H NMR (400 MHZ, DMSO-d₆) δ11.91 (br. s, 1H), 10.98 (s, 1H), 8.14 (s, 1H), 8.07 (s, 1H), 7.41 (s, 1H), 6.68 (s, 2H), 5.78 (s, 1H), 3.87 (s, 3H), 3.78 (s, 6H), 3.01 (s, 3H), 1.91-1.81 (m, 1H), 1.45-1.41 (m, 6H), 0.96-0.87 (m, 2H), 0.70-0.62 (m, 2H); LCMS m/z 558.3 (M+H)⁺.

### Example 272 and 273: N-{6-[(3-cyclopropyl-1H-pyrazol-5-yl)amino]-5-methoxy-1,2-benzoxazol-3-yl}-2,6-dimethoxy-4-(1-methoxyethyl)benzene-1-sulfonamide

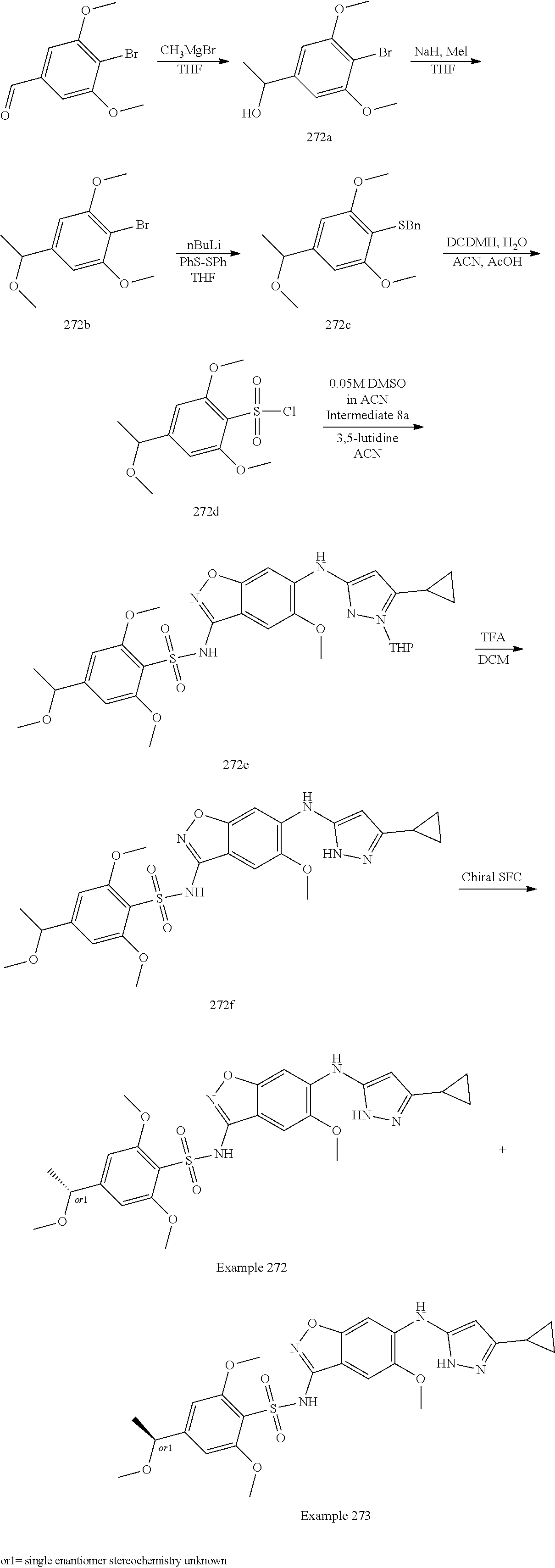

### Step 1: Synthesis of 1-(4-bromo-3,5-dimethoxyphenyl) ethan-1-ol (272a)

To a solution of 4-bromo-3,5-dimethoxybenzaldehyde (1.00 g, 4.08 mmol) in THF (13.6 mL) was added methylmagnesium bromide (730 mg, 6.12 mmol) dropwise at −10° C. while stirring. The mixture was stirred at 0° C. for 30 mins, then quenched with water, extracted with EtOAc (3×12 mL) and washed with water (10 mL). The combined organic layers were dried over Na₂SO₄, concentrated under reduced pressure, and purified by column chromatography (20 g silica gel, 0-34% EtOAc/Pet. ether) to afford 272a (1.00 g, 94%) as a white solid. ¹H NMR (400 MHz, DMSO-d₆) δ 6.71 (s, 2H), 5.26 (d, J=4.3 Hz, 1H), 4.71 (dd, J=4.4, 6.3 Hz, 1H), 3.82 (s, 6H), 1.33 (d, J=6.5 Hz, 3H).

### Step 2: Synthesis of 2-bromo-1,3-dimethoxy-5-(1-methoxyethyl)benzene (272b)

To a stirred solution of 1-(4-bromo-3,5-dimethoxyphenyl) ethan-1-ol 272a (700 mg, 2.68 mmol) in THF (8.94 mL) was added sodium hydride (161 mg, 4.02 mmol) at 0° C., and the resulting mixture was allowed to stir for 20 mins. Methyl iodide (533 mg, 3.74 mmol) was added, and the mixture was stirred at 30° C. for 16 hrs. The reaction was quenched with water, extracted with EtOAc (3×12 mL) and washed with water (10 mL). The combined organic layers were dried over Na₂SO₄, concentrated under reduced pressure, and purified by column chromatography (12 g silica gel, 0-12% EtOAc/Pet. ether) to afford 272b (695 mg, 94%) as a colorless oil. 1H NMR (400 MHZ, DMSO-d₆) δ 6.66 (s, 2H), 4.31 (q, J=6.4 Hz, 1H), 3.83 (s, 6H), 3.15 (s, 3H), 1.34 (d, J=6.4 Hz, 3H).

### Step 3: Synthesis of 2-(benzylsulfanyl)-1,3-dimethoxy-5-(1-methoxyethyl)benzene (272c)

To a stirred solution of 2-bromo-1,3-dimethoxy-5-(1-methoxyethyl)benzene 272b (495 mg, 1.80 mmol) in anhydrous THF (8.86 mL) was added dropwise n-butyllithium (144 mg, 2.25 mmol) at-65° C. under nitrogen. The mixture was stirred for 1 hr at −65° C., then diphenyl disulfide (532 mg, 2.16 mmol) was added portionwise under nitrogen at −65° C. The mixture was stirred at −65° C. for 2 hrs, then quenched with MeOH. The material was purified by flash column chromatography (20 g silica gel, 0-16% EtOAc/Pet. ether) to afford 272c (380 mg, 66%) as a colorless oil. ¹H NMR (400 MHZ, DMSO-d₆) δ 7.29-7.10 (m, 5H), 6.56 (s, 2H), 4.27 (q, J=6.4 Hz, 1H), 3.94 (s, 2H), 3.76 (s, 6H), 3.13 (s, 3H), 1.32 (d, J=6.4 Hz, 3H).

### Step 4: Synthesis of 2,6-dimethoxy-4-(1-methoxyethyl)benzene-1-sulfonyl chloride (272d)

To a stirred solution of 2-(benzylsulfanyl)-1,3-dimethoxy-5-(1-methoxyethyl)benzene 272c (380 mg, 1.19 mmol) in AcOH (0.25 mL), water (0.1 mL) and ACN (4.0 mL) was added 1,3-dichloro-5,5-dimethylhydantoin (329 mg, 1.67 mmol) at 25° C. The mixture was stirred for 20 mins, then poured into water (20 mL) and extracted with EtOAc (2×15 mL). The combined organic layers were washed with water (10 mL) and brine (10 mL), dried over Na₂SO₄, concentrated under reduced pressure, and purified by column chromatography (20 g silica gel, 0-25% EtOAc/Pet. ether) to afford 272d (200 mg, 60%) as a colorless oil. ¹H NMR (400 MHZ, DMSO-d₆) δ 6.57 (s, 2H), 4.27 (q, J=6.3 Hz, 1H), 3.73 (s, 6H), 3.15 (s, 3H), 1.32 (d, J=6.4 Hz, 3H).

### Step 5: Synthesis of N-(6-{[5-cyclopropyl-1-(oxan-2-yl)-1H-pyrazol-3-yl]amino}-5-methoxy-1,2-benzoxazol-3-yl)-2,6-dimethoxy-4-(1-methoxyethyl)benzene-1-sulfonamide (272e)

To a solution of 272d (197 mg, 0.67 mmol) and/6-[5-cyclopropyl-1-(oxan-2-yl)-1H-pyrazol-3-yl]-5-methoxy-1,2-benzoxazole-3,6-diamine (8a) (165 mg, 0.447 mmol) in 0.05 N DMSO in ACN (1.74 mg, 0.0223 mmol, 0.447 mL) was added 3,5-dimethylpyridine (144 mg, 1.34 mmol) in ACN (1.49 mL). The suspension was stirred at 60° C. for 16 hrs. This mixture was combined with a separate batch created using an identical procedure on an 80 mg scale, and the combined material was purified by column chromatography (20 g silica gel, 0-73% EtOAc/Pet. ether) to afford 272e (170 mg, 61%) as a white solid. LCMS m/z 628.2 (M+H)⁺.

### Step 6: Synthesis of N-{6-[(5-cyclopropyl-1H-pyrazol-3-yl)amino]-5-methoxy-1,2-benzoxazol-3-yl}-2,6-dimethoxy-4-(1-methoxyethyl)benzene-1-sulfonamide (Example 178) and N-{6-[(3-cyclopropyl-1H-pyrazol-5-yl)amino]-5-methoxy-1,2-benzoxazol-3-yl}-2,6-dimethoxy-4-(1-methoxyethyl)benzene-1-sulfonamide (Example 272)

A solution of N-(6-{[5-cyclopropyl-1-(oxan-2-yl)-1H-pyrazol-3-yl]amino}-5-methoxy-1,2-benzoxazol-3-yl)-2,6-dimethoxy-4-(1-methoxyethyl)benzene-1-sulfonamide (272e) in DCM (1.0 mL) and TFA (2.0 mL) was stirred at rt for 4 hrs. The mixture was purified by column chromatography (12 g silica gel, 0-100% EtOAc/Pet. ether) to afford a racemic mixture of N-{6-[(5-cyclopropyl-1H-pyrazol-3-yl)amino]-5-methoxy-1,2-benzoxazol-3-yl}-2,6-dimethoxy-4-(1-methoxyethyl)benzene-1-sulfonamide (272f) as a white solid (130 mg, 88%). The enantiomers were separated by chiral SFC (Daicel ChiralPak IG 250×30 mm column, 10 mm particle size Mobile phase A: CO₂ Mobile phase B: EtOH+0.1% NH₃H₂O 60% isocratic flow rate: 80 mL/mins).

### N-{6-[(5-cyclopropyl-1H-pyrazol-3-yl)amino]-5-methoxy-1,2-benzoxazol-3-yl}-2,6-dimethoxy-4-(1-methoxyethyl)benzene-1-sulfonamide (Example 272)

The first eluting peak was isolated as a white solid and further purified by preparative HPLC (Phenomenex Gemini NX 150×30 mm column, 5 mm particle size Mobile phase A: water+0.1% ammonium hydroxide Mobile phase B: ACN 6-46% over 9 mins, flow rate: 60 mL/mins) to afford Example 272 as a white solid (13.8 mg, 9%). ¹H NMR (400 MHZ, DMSO-d₆) δ 11.90 (s, 1H), 10.94 (br. s, 1H), 8.12 (s, 1H), 8.03 (s, 1H), 7.40 (s, 1H), 6.66 (s, 2H), 5.78 (s, 1H), 4.30 (q, J=6.2 Hz, 1H), 3.87 (s, 3H), 3.77 (s, 6H), 3.15 (s, 3H), 1.92-1.79 (m, 1H), 1.31 (d, J=6.5 Hz, 3H), 0.97-0.86 (m, 2H), 0.73-0.61 (m, 2H); LCMS m/z 544.2 (M+H)⁺; [a]_{D}²²=+26.0° (c 0.1, MeOH).

### N-{6-[(5-cyclopropyl-1H-pyrazol-3-yl)amino]-5-methoxy-1,2-benzoxazol-3-yl}-2,6-dimethoxy-4-(1-methoxyethyl)benzene-1-sulfonamide (Example 273)

The second eluting peak was isolated as a white solid and further purified by preparative HPLC (Phenomenex Gemini NX 150×30 mm column, 5 mm particle size Mobile phase A: water+0.1% ammonium hydroxide Mobile phase B: ACN 6-46% over 9 mins, flow rate: 60 mL/mins) to afford Example 273 as a white solid (10.8 mg, 7%). ¹H NMR (400 MHZ, DMSO-d₆) δ11.90 (s, 1H), 10.94 (br. s, 1H), 8.12 (s, 1H), 8.03 (s, 1H), 7.40 (s, 1H), 6.66 (s, 2H), 5.78 (s, 1H), 4.30 (q, J=6.4 Hz, 1H), 3.87 (s, 3H), 3.77 (s, 6H), 3.15 (s, 3H), 1.91-1.81 (m, 1H), 1.31 (d, J=6.5 Hz, 3H), 0.96-0.88 (m, 2H), 0.70-0.62 (m, 2H); LCMS m/z 544.2 (M+H)⁺; [a]_{D}22=−36.7° (c 0.1, MeOH).

### Example 274 and 275: N-{6-[(3-cyclopropyl-1H-pyrazol-5-yl)amino]-5-methoxy-1,2-benzoxazol-3-yl}-4-(1,2-dimethoxyethyl)-2,6-dimethoxybenzene-1-sulfonamide

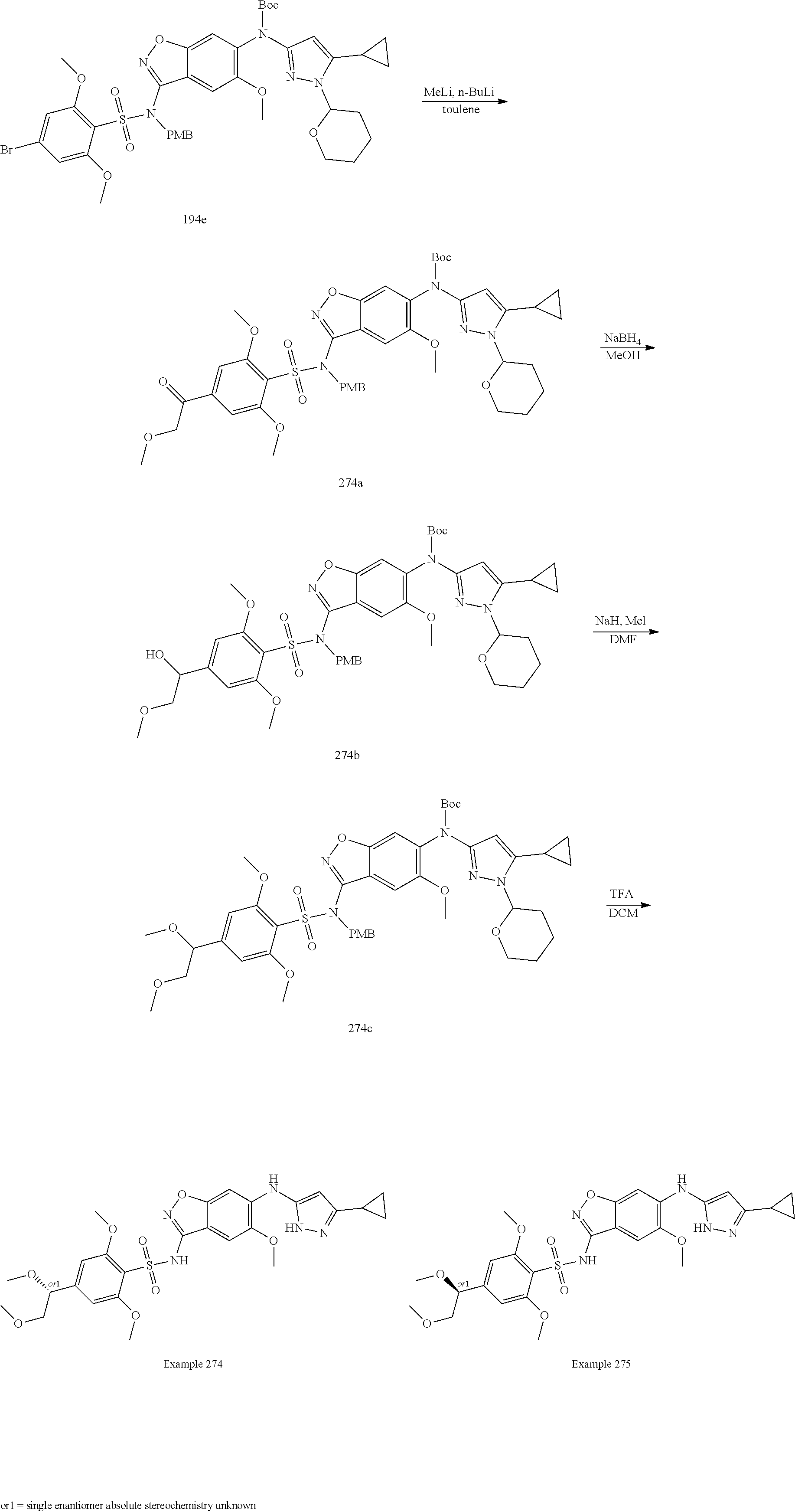

### Step 1: Synthesis of tert-butyl [5-cyclopropyl-1-(oxan-2-yl)-1H-pyrazol-3-yl](3-{[2,6-dimethoxy-4-(methoxyacetyl)benzene-1-sulfonyl][(4-methoxyphenyl)methyl]amino}-5-methoxy-1,2-benzoxazol-6-yl)carbamate (274a)

To a solution of tert-butyl (3-{(4-bromo-2,6-dimethoxybenzene-1-sulfonyl) [(4-methoxyphenyl)methyl]amino}-5-methoxy-1,2-benzoxazol-6-yl) [5-cyclopropyl-1-(oxan-2-yl)-1H-pyrazol-3-yl]carbamate 194e (850 mg, 0.978 mmol) in toluene (24.5 mL) under nitrogen at −70° C. was added methyllithium (1.6 M, 43 mg, 1.96 mmol) dropwise over 15 mins. The reaction mixture was stirred at −70° C. for 1 hr and n-butyllithium (2.5 M, 125 mg, 1.96 mmol) was then added dropwise. After stirring at −70° C. for another 1 hr, N,2-dimethoxy-N-methylacetamide (782 mg 5.87 mmol) was added dropwise. The resulting mixture was stirred at −70° C. for 2 hrs and then allowed to warm to rt and quenched with NH₄Cl (10 mL). The aqueous phase was extracted with EtOAc (20 mL×3). The combined organic layer was washed with brine (20 mL), dried over Na₂SO₄, filtered and concentrated under reduced pressure. The resulting residue was purified by silica gel column chromatography (eluting with 0-80% EtOAc in Pet. ether) to give 274a (450 mg, 53.4%) as a yellow oil. LCMS m/z 862.4 (M+H)⁺.

### Step 2: Synthesis of tert-butyl [5-cyclopropyl-1-(oxan-2-yl)-1H-pyrazol-3-yl](3-{[4-(1-hydroxy-2-methoxyethyl)-2,6-dimethoxybenzene-1-sulfonyl][(4-methoxyphenyl)methyl]amino}-5-methoxy-1,2-benzoxazol-6-yl) carbamate (274b)

To a red suspension of tert-butyl [5-cyclopropyl-1-(oxan-2-yl)-1H-pyrazol-3-yl](3-{[2,6-dimethoxy-4-(methoxyacetyl)benzene-1-sulfonyl][(4-methoxyphenyl)methyl]amino}-5-methoxy-1,2-benzoxazol-6-yl) carbamate 274a (0.7 g, 0.8 mmol) in MeOH (5.41 mL) at 0° C. was added sodium borohydride (150 mg, 3.96 mmol). The reaction mixture was stirred at 15° C. for 2 hrs and was then quenched with sat. aq. NH₄Cl (5 mL). The aqueous phase was extracted with DCM (5 mL×2). The combined organic layer was dried over Na₂SO₄, filtered, and concentrated under reduced pressure. The resulting crude (500 mg) was obtained as a yellow oil, which was used in the next step without further purification. 300 mg was used in the next step without further purification and 200 mg was purified by preparative HPLC (C18 150×40 mm column, Mobile phase A: water+NH₃H₂O+NH₄HCO₃, Mobile phase B: ACN 53-73% over 9 mins, flow rate 30 mL/min) to give 274b (87.6 mg) as a white solid. ¹H NMR (400 MHZ, DMSO-d₆) δ 7.55-7.48 (m, 1H), 7.34-7.28 (m, 2H), 7.22-7.16 (m, 1H), 6.87-6.81 (m, 2H), 6.80-6.76 (m, 2H), 6.10-5.92 (m, 1H), 5.69-5.59 (m, 1H), 5.42-5.32 (m, 1H), 5.04-4.90 (m, 2H), 4.78-4.66 (m, 1H), 3.88-3.76 (m, 1H), 3.71-3.65 (m, 13H), 3.58-3.51 (m, 1H), 3.28-3.27 (m, 3H), 2.00-1.88 (m, 2H), 1.87-1.79 (m, 1H), 1.70-1.35 (m, 5H), 1.33-1.24 (m, 9H), 1.02-0.88 (m, 2H), 0.74-0.57 (m, 2H); LCMS m/z 864.5 (M+H)⁺.

### Step 3: Synthesis of tert-butyl [5-cyclopropyl-1-(oxan-2-yl)-1H-pyrazol-3-yl](3-{[4-(1,2-dimethoxyethyl)-2,6-dimethoxybenzene-1-sulfonyl][(4-methoxyphenyl)methyl]amino}-5-methoxy-1,2-benzoxazol-6-yl)carbamate (274c)

To a solution of tert-butyl [5-cyclopropyl-1-(oxan-2-yl)-1H-pyrazol-3-yl](3-{[4-(1-hydroxy-2-methoxyethyl)-2,6-dimethoxybenzene-1-sulfonyl][(4-methoxyphenyl)methyl]amino}-5-methoxy-1,2-benzoxazol-6-yl)carbamate 274b (300 mg, 0.347 mmol) in DMF (3.47 mL) at 0° C. was added sodium hydride (60% in mineral oil, 20 mg, 0.5 mmol). The reaction mixture was stirred at 0° C. for 1 hrs and then methyl iodide (246 mg, 1.74 mmol) was added. The mixture was allowed to warm to 15° C. and was stirred at that temperature for 3 hrs. The reaction was diluted with H₂O (5 mL) and extracted with EtOAc (20 mL×3). The combined organic phase was washed with brine (20 mL×3), dried over Na₂SO₄ and concentrated to give crude 274c (305 mg, crude) as a yellow oil which was used directly in the next step. LCMS m/z 878.6 (M+H)⁺.

### Step 4: Synthesis of N-{6-[(5-cyclopropyl-1H-pyrazol-3-yl)amino]-5-methoxy-1,2-benzoxazol-3-yl}-4-(1,2-dimethoxyethyl)-2,6-dimethoxybenzene-1-sulfonamide (Example 274)

To a solution of tert-butyl [5-cyclopropyl-1-(oxan-2-yl)-1H-pyrazol-3-yl](3-{[4-(1,2-dimethoxyethyl)-2,6-dimethoxybenzene-1-sulfonyl][(4-methoxyphenyl)methyl]amino}-5-methoxy-1,2-benzoxazol-6-yl) carbamate 274c (305 mg, 0.347 mmol) in DCM (2 mL) was added TFA (4.0 mL) at 15° C. The reaction mixture was stirred at ˜15° C. for 16 hrs and then concentrated under reduced pressure. The resulting residue was purified by flash silica gel column chromatography (eluting with 0-90% EtOAc in Pet. ether) to give a racemic mixture of 274 and 275 (150 mg, 75%) as a yellow oil. LCMS m/z 574.2 (M+H)⁺. The enantiomers were separated by chiral SFC (50% methanol with 0.1% ammonium hydroxide in CO₂; 150 mL/min flow rate; Column: Daicel Chiralpak® AD (250×30 mm; 10 μm)).

### N-{6-[(5-cyclopropyl-1H-pyrazol-3-yl)amino]-5-methoxy-1,2-benzoxazol-3-yl}-4-(1,2-dimethoxyethyl)-2,6-dimethoxybenzene-1-sulfonamide (Example 274)

The first eluting peak was lyophilized and gave 273 (46.08 mg, 23% yield, 100% ee) as a white solid. ¹H NMR (400 MHZ, METHANOL-d₄) δ 7.77-7.49 (m, 1H), 7.42-7.25 (m, 1H), 6.75-6.69 (m, 2H), 5.84-5.68 (m, 1H), 4.44-4.31 (m, 1H), 3.99-3.96 (m, 3H), 3.90-3.84 (m, 6H), 3.57-3.44 (m, 2H), 3.35-3.34 (m, 3H), 3.30-3.28 (m, 3H), 1.98-1.83 (m, 1H), 1.03-0.95 (m, 2H), 0.79-0.73 (m, 2H); LCMS m/z 574.2 (M+H)⁺; [a]_{D}²⁶=+61.33° (c 0.001, CH₃OH).

### N-{6-[(5-cyclopropyl-1H-pyrazol-3-yl)amino]-5-methoxy-1,2-benzoxazol-3-yl}-4-(1,2-dimethoxyethyl)-2,6-dimethoxybenzene-1-sulfonamide (Example 275)

The second eluting peak was lyophilized to give 274 (56.25 mg, 28% yield, 92.2% ee) as a white solid. ¹H NMR (400 MHZ, METHANOL-d₄) δ 7.70-7.56 (m, 1H), 7.40-7.22 (m, 1H), 6.76-6.66 (m, 2H), 5.83-5.71 (m, 1H), 4.44-4.31 (m, 1H), 4.01-3.94 (m, 3H), 3.91-3.83 (m, 6H), 3.57-3.43 (m, 2H), 3.32-3.31 (m, 3H), 3.30-3.26 (m, 3H), 1.99-1.81 (m, 1H), 1.05-0.91 (m, 2H), 0.86-0.67 (m, 2H); LCMS m/z 574.2 (M+H)⁺; [a]_{D}²⁶=−34.67° (c 0.001, CH₃OH).

### Example 276: N-{6-[(3-cyclopropyl-1H-pyrazol-5-yl)amino]-5-methoxy-1,2-benzoxazol-3-yl}-2,6-dimethoxy-4-(oxetan-2-yl)benzene-1-sulfonamide

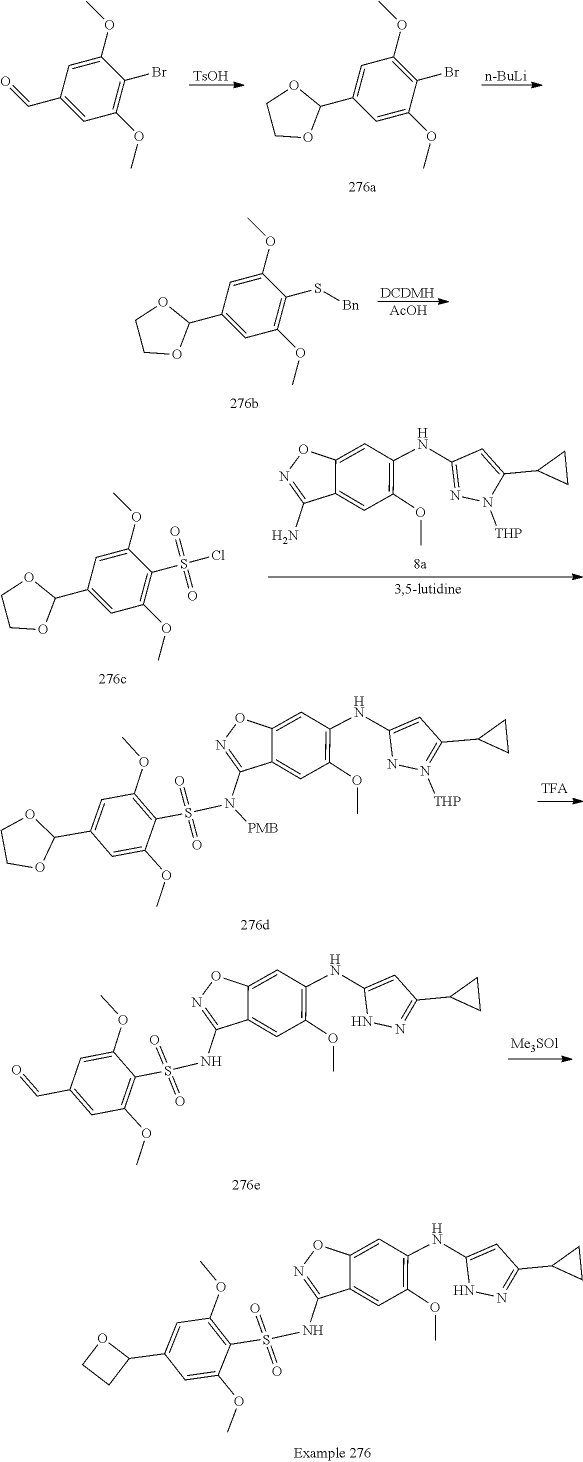

### Step 1: Synthesis of 2-(4-bromo-3,5-dimethoxyphenyl)-1,3-dioxolane (276a)

A mixture of 4-bromo-3,5-dimethoxybenzaldehyde (4000 mg, 16.32 mmol), ethylene glycol (2030 mg, 32.6 mmol) and p-toluenesulfonic acid monohydrate (621 mg, 3.26 mmol) in toluene (65 mL) was stirred at 100° C. for 16 hrs. After completion, the reaction mixture was washed with a sat. aq. solution of NaHCO₃ (100 mL). The organic layer was dried over Na₂SO₄, filtered and concentrated under reduced pressure. The crude residue was purified by silica gel column chromatography (eluting with 0-20% EtOAC in Pet. ether) to give 276a (4000 mg, 84.8%) as colorless oil. ¹H NMR (400 MHZ, CDCl₃) δ 6.79-6.67 (m, 2H), 5.87-5.76 (m, 1H), 4.18-4.05 (m, 4H), 3.97-3.91 (m, 6H).

### Step 2: Synthesis of 2-(3,5-dimethoxy-4-{[(4-methoxyphenyl)methyl]sulfanyl}phenyl)-1,3-dioxolane (276b)

To a stirred solution of 2-(4-bromo-3,5-dimethoxyphenyl)-1,3-dioxolane 276a (2000 mg, 6.917 mmol) in dry THF (30 mL) at −78° C. under nitrogen was added n-butyllithium (2.5 M, 620 mg, 9.68 mmol) dropwise. The solution became gray. After addition, the reaction mixture was stirred at −78° C. for 1 hr. A solution of benzyl disulfide (2220 mg, 8.99 mmol) in THF (5 mL) under nitrogen was then added dropwise at −78° C. and the resulting mixture was stirred at that temperature for 2 hrs. The reaction was quenched with water and extracted with EtOAc. The organic phase was washed with brine, dried over Na₂SO₄ and concentrated under reduced pressure. The resulting crude was purified by flash silica gel column chromatography (eluting with 0-20% EtOAc in Pet. ether) to give 276b (1500 mg, 73%) as a colorless oil. ¹H NMR (400 MHz, CDCl₃) δ 7.24-7.14 (m, 5H), 6.71-6.65 (m, 2H), 5.82-5.77 (m, 1H), 4.19-4.04 (m, 4H), 4.02-3.97 (m, 2H), 3.88-3.82 (m, 6H).

### Step 3: Synthesis of 4-(1,3-dioxolan-2-yl)-2,6-dimethoxybenzene-1-sulfonyl chloride (276c)

To a stirred solution of 2-(3,5-dimethoxy-4-{[(4-methoxyphenyl)methyl]sulfanyl}phenyl)-1,3-dioxolane 276b (1500 mg, 5.06 mmol) in a mixture of AcOH (4.8 mL), ACN (30 mL) and H₂O (3.2 mL) at 0˜5° C. was added 1,3-dichloro-5,5-dimethylhydantoin (1400 mg, 7.09 mmol). The reaction mixture was stirred at that temperature for 30 min and was then diluted with EtOAc (5 mL×3). The combined organic phase was washed with brine (8 mL), dried over Na₂SO₄, filtered, and concentrated under reduced pressure. The resulting residue was purified by flash silica gel column chromatography (eluting with 0-30% EtOAc in Pet. ether) to give 276c (700 mg, 44.8%) as a colorless oil. ¹H NMR (400 MHZ, CDCl₃) δ 6.84-6.75 (m, 2H), 5.86-5.78 (m, 1H), 4.15-4.06 (m, 4H), 4.05-3.98 (m, 6H).

### Step 4: Synthesis of N-(6-{[5-cyclopropyl-1-(oxan-2-yl)-1H-pyrazol-3-yl]amino}-5-methoxy-1,2-benzoxazol-3-yl)-4-(1,3-dioxolan-2-yl)-2,6-dimethoxybenzene-1-sulfonamide (276d)

276d was made in a similar manner as 270b using N6-[5-cyclopropyl-1-(oxan-2-yl)-1H-pyrazol-3-yl]-5-methoxy-1,2-benzoxazole-3,6-diamine 8a (700 mg, 1.89 mmol) and 4-(1,3-dioxolan-2-yl)-2,6-dimethoxybenzene-1-sulfonyl chloride 276c (700 mg, 2.27 mmol). The resulting crude was purified by flash silica gel column chromatography (eluting with 0-80% EtOAc in Pet ether) to give 276d (1000 mg, 82%) as a yellow solid. LCMS m/z 642.3 (M+H)⁺.

### Step 5: Synthesis of N-{6-[(5-cyclopropyl-1H-pyrazol-3-yl)amino]-5-methoxy-1,2-benzoxazol-3-yl}-4-formyl-2,6-dimethoxybenzene-1-sulfonamide (276e)

To a solution of N-(6-{[5-cyclopropyl-1-(oxan-2-yl)-1H-pyrazol-3-yl]amino}-5-methoxy-1,2-benzoxazol-3-yl)-4-(1,3-dioxolan-2-yl)-2,6-dimethoxybenzene-1-sulfonamide 276d (1000 mg, 1.56 mmol) in DCM (5 mL) at 25° C. was added TFA (5 mL) dropwise. The reaction mixture was stirred at that temperature for 6 hrs and was then concentrated under reduced pressure. The resulting residue was purified by flash silica gel column chromatography (eluting with 0-80% EtOAc in Pet ether) to give 276e (200 mg, 25%) as a yellow solid. LCMS m/z 514.1 (M+H)⁺.

### Step 6: Synthesis of N-{6-[(3-cyclopropyl-1H-pyrazol-5-yl)amino]-5-methoxy-1,2-benzoxazol-3-yl}-2,6-dimethoxy-4-(oxetan-2-yl)benzene-1-sulfonamide (Example 276)

Trimethylsulfoxonium iodide (600 mg, 2.73 mmol) in t-BuOH (6 mL) was treated with t-BuOK (306 mg, 2.73 mmol). The reaction mixture was stirred at 50° C. for 1 hr and N-{6-[(5-cyclopropyl-1H-pyrazol-3-yl)amino]-5-methoxy-1,2-benzoxazol-3-yl}-4-formyl-2,6-dimethoxybenzene-1-sulfonamide 276e (350 mg, 0.682 mmol) was then added dropwise. The resulting mixture was stirred at 50° C. for 16 hrs and then concentrated under reduced pressure. The resulting residue was dissolved in MeOH (3 mL) and purified by preparative HPLC (Phenomenex Gemini NX 150×30 mm column, 5 mm particle size, Mobile phase A: water+NH₃H₂O+NH₄HCO₃, Mobile phase B: ACN 18-38% over 9 mins, flow rate 30 mL/min) to give a white solid (10 mg). The latter was repurified by preparative TLC (eluting with a mixture of MeOH in DCM (1:15)) to afford a yellow oil, that was subsequently lyophilized to give Example 276 (1 mg, 0.27%) as a white solid. ¹H NMR (400 MHZ, METHANOL-d₄) δ 7.65-7.50 (m, 1H), 7.26-7.15 (m, 1H), 6.61-6.37 (m, 2H), 5.70-5.54 (m, 1H), 4.70-4.69 (m, 1H), 3.90-3.82 (m, 3H), 3.79-3.66 (m, 8H), 3.55-3.47 (m, 1H), 2.92-2.85 (m, 1H), 1.85-1.75 (m, 1H), 0.92-0.85 (m, 2H), 0.67-0.56 (m, 2H); LCMS m/z 542.1, 542.4 (M+H)⁺.

### Biological Assay Section

### Materials

KAT enzymes were recombinantly expressed in insect cells using a baculovirus expression system and purified at Pfizer, La Jolla, as described for KAT5, KAT6A, and KAT8 by (Sharma, et al., Discovery of a highly potent, selective, orally bioavailable inhibitor of KAT6A/B histone acetyltransferases with efficacy against KAT6A-high ER+ breast cancer. Cell Chemical Biology, 2023, 30 (10): 1191-1210). Histone H3 (1-21) peptide (ARTKQTARKSTGGKAPRKQLA; SEQ ID NO: 1) and Histone H4 (1-21) peptide (SGRGKGGKGLGKGGAKRHRKV; SEQ ID NO: 2) were purchased from CPC Scientific (Sunnyvale, CA). Acetyl coenzyme A was purchased from Sigma-Aldrich (St. Louis, MO). All other biochemical reagents were purchased from Sigma-Aldrich or ThermoFisher Scientific (Waltham, MA).

### KAT5:

A codon optimized DNA sequence encoding amino acid residues 1-513 (Uniprot Q92993-1) of human KAT5 gene was synthesized de novo (Genscript USA Inc., Piscataway, New Jersey) and ligated into a modified pFastBac vector containing N-terminal hexahistidine affinity tag, followed by a tobacco etch virus (TEV) protease cleavage site. The resulting protein sequence is listed below:

(SEQ ID NO. 3)
MASHHHHHHDYDGATTENLYFQGSMAEVGEIIEGCRLPVLRRNQDNEDE
WPLAEILSVKDISGRKLFYVHYIDFNKRLDEWVTHERLDLKKIQFPKKE
AKTPTKNGLPGSRPGSPEREVPASAQASGKTLPIPVQITLRFNLPKERE
AIPGGEPDQPLSSSSCLQPNHRSTKRKVEVVSPATPVPSETAPASVFPQ
NGAARRAVAAQPGRKRKSNCLGTDEDSQDSSDGIPSAPRMTGSLVSDRS
HDDIVTRMKNIECIELGRHRLKPWYFSPYPQELTTLPVLYLCEFCLKYG
RSLKCLQRHLTKCDLRHPPGNEIYRKGTISFFEIDGRKNKSYSQNLCLL
AKCFLDHKTLYYDTDPFLFYVMTEYDCKGFHIVGYFSKEKESTEDYNVA
CILTLPPYQRRGYGKLLIEFSYELSKVEGKTGTPEKPLSDLGLLSYRSY
WSQTILEILMGLKSESGERPQITINEISEITSIKKEDVISTLQYLNLIN
YYKGQYILTLSEDIVDGHERAMLKRLLRIDSKCLHFTPKDWSKRGKW

### KAT6A:

A codon optimized DNA sequence encoding amino acid residues 1-2004 (Uniprot Q92794-1) of human KAT6A gene was synthesized de novo (Genscript) and ligated into a modified pFastBac vector containing N-terminal Flag affinity tag, followed by a TEV protease cleavage site. The resulting protein sequence is listed below:

(SEQ ID NO. 4)
DWNKLIKRAVEGLAESGGSTLKSIERFLKGQKDVSALFGGSAASGFHQQ
LRLAIKRAIGHGRLLKDGPLYRLNTKATNVDGKESCESLSCLPPVSLLP
HEKDKPVAEPIPICSFQLGTKEQNREKKPEELISCADCGNSGHPSCLKF
SPELTVRVKALRWQCIECKTCSSCRDQGKNADNMLFCDSCDRGFHMECC
DPPLTRMPKGMWICQICRPRKKGRKLLQKKAAQIKRRYTNPIGRPKNRL
KKQNTVSKGPFSKVRTGPGRGRKRKITLSSQSASSSSEEGYLERIDGLD
FCRDSNVSLKFNKKTKGLIDGLTKFFTPSPDGRKARGEVVDYSEQYRIR
KRGNRKSSTSDWPTDNQDGWDGKQENEERLFGSQEIMTEKDMELFRDIQ
EQALQKVGVTGPPDPQVRCPSVIEFGKYEIHTWYSSPYPQEYSRLPKLY
LCEFCLKYMKSRTILQQHMKKCGWFHPPANEIYRKNNISVFEVDGNVST
IYCQNLCLLAKLFLDHKTLYYDVEPFLFYVLTQNDVKGCHLVGYFSKEK
HCQQKYNVSCIMILPQYQRKGYGRFLIDFSYLLSKREGQAGSPEKPLSD
LGRLSYMAYWKSVILECLYHQNDKQISIKKLSKLTGICPQDITSTLHHL
RMLDFRSDQFVIIRREKLIQDHMAKLQLNLRPVDVDPECLRWTPVIVSN
SVVSEEEEEEAEEGENEEPQCQERELEISVGKSVSHENKEQDSYSVESE
KKPEVMAPVSSTRLSKQVLPHDSLPANSQPSRRGRWGRKNRKTQERFGD
KDSKLLLEETSSAPQEQYGECGEKSEATQEQYTESEEQLVASEEQPSQD
GKPDLPKRRLSEGVEPWRGQLKKSPEALKCRLTEGSERLPRRYSEGDRA
VLRGFSESSEEEEEPESPRSSSPPILTKPTLKRKKPFLHRRRRVRKRKH
HNSSVVTETISETTEVLDEPFEDSDSERPMPRLEPTFEIDEEEEEEDEN
ELFPREYFRRLSSQDVLRCQSSSKRKSKDEEEDEESDDADDTPILKPVS
LLRKRDVKNSPLEPDTSTPLKKKKGWPKGKSRKPIHWKKRPGRKPGFKL
SREIMPVSTQACVIEPIVSIPKAGRKPKIQESEETVEPKEDMPLPEERK
EEEEMQAEAEEAEEGEEEDAASSEVPAASPADSSNSPETETKEPEVEEE
EEKPRVSEEQRQSEEEQQELEEPEPEEEEDAAAETAQNDDHDADDEDDG
HLESTKKKELEEQPTREDVKEEPGVQESFLDANMQKSREKIKDKEETEL
DSEEEQPSHDTSVVSEQMAGSEDDHEEDSHTKEELIELKEEEEIPHSEL
DLETVQAVQSLTQEESSEHEGAYQDCEETLAACQTLQSYTQADEDPQMS
MVEDCHASEHNSPISSVQSHPSQSVRSVSSPNVPALESGYTQISPEQGS
LSAPSMQNMETSPMMDVPSVSDHSQQVVDSGFSDLGSIESTTENYENPS
SYDSTMGGSICGNSSSQSSCSYGGLSSSSSLTQSSCVVTQQMASMGSSC
SMMQQSSVQPAANCSIKSPQSCVVERPPSNQQQQPPPPPPQQPQPPPPQ
PQPAPQPPPPQQQPQQQPQPQPQQPPPPPPPQQQPPLSQCSMNNSFTPA
PMIMEIPESGSTGNISIYERIPGDFGAGSYSQPSATFSLAKLQQLTNTI
MDPHAMPYSHSPAVTSYATSVSLSNTGLAQLAPSHPLAGTPQAQATMTP
PPNLASTTMNLTSPLLQCNMSATNIGIPHTQRLQGQMPVKGHISIRSKS
APLPSAAAHQQQLYGRSPSAVAMQAGPRALAVQRGMNMGVNLMPTPAYN
VNSMNMNTLNAMNSYRMTQPMMNSSYHSNPAYMNQTAQYPMQMQMGMMG
SQAYTQQPMQPNPHGNMMYTGPSHHSYMNAAGVPKQSLNGPYMRR

### KAT7:

A codon optimized DNA sequence encoding amino acid residues 1-611 (Uniprot O95251-1) of human KAT7 gene was synthesized de novo (Genscript) and ligated into a modified pFastBac vector containing C-terminal hexahistidine tag, preceded by a TEV protease cleavage site. The resulting protein sequence is listed below:

(SEQ ID NO. 5
MASDYKDDDDKGATTENLYFQGSMVKLANPLYTEWILEAIKKVKKQKQR
PSEERICNAVSSSHGLDRKTVLEQLELSVKDGTILKVSNKGLNSYKDPD
NPGRIALPKPRNHGKLDNKQNVMPRRKRNAGSSSDGTEDSDFSTDLEHT
DSSESDGTSRRSARVTRSSARLSQSSQDSSPVRNLQSFGTEEPAYSTRR
VTRSQQQPTPVTPKKYPLRQTRSSGSETEQVVDFSDRETKNTADHDESP
PRTPTGNAPSSESDIDISSPNVSHDESIAKDMSLKDSGSDLSHRPKRRR
FHESYNFNMKCPTPGCNSLGHLTGKHERHFSISGCPLYHNLSADECKVR
AQSRDKQIEERMLSHRQDDNNRHATRHQAPTERQLRYKEKVAELRKKRN
SGLSKEQKEKYMEHRQTYGNTREPLLENLTSEYDLDLFRRAQARASEDL
EKLRLQGQITEGSNMIKTIAFGRYELDTWYHSPYPEEYARLGRLYMCEF
CLKYMKSQTILRRHMAKCVWKHPPGDEIYRKGSISVFEVDGKKNKIYCQ
NLCLLAKLFLDHKTLYYDVEPFLFYVMTEADNTGCHLIGYFSKEKNSFL
NYNVSCILTMPQYMRQGYGKMLIDFSYLLSKVEEKVGSPERPLSDLGLI
SYRSYWKEVLLRYLHNFQGKEISIKEISQETAVNPVDIVSTLQALQMLK
YWKGKHLVLKRQDLIDEWIAKEAKRSNSNKTMDPSCLKWTPPKGTLENL
YFQGHHHHHH

### KAT8:

A codon optimized DNA sequence encoding amino acid residues 1-458 (Uniprot Q9H7Z6-1) of human KAT8 gene was synthesized de novo (Genscript) and ligated into a modified pFastBac vector containing N-terminal hexahistidine tag, followed by a TEV protease cleavage site. The resulting protein sequence is listed below:

(SEQ ID NO. 6)
MASHHHHHHDYDGATTENLYFQGSMAAQGAAAAVAAGTSGVAGEGEPGP
GENAAAEGTAPSPGRVSPPTPARGEPEVTVEIGETYLCRRPDSTWHSAE
VIQSRVNDQEGREEFYVHYVGFNRRLDEWVDKNRLALTKTVKDAVQKNS
EKYLSELAEQPERKITRNQKRKHDEINHVQKTYAEMDPTTAALEKEHEA
ITKVKYVDKIHIGNYEIDAWYFSPFPEDYGKQPKLWLCEYCLKYMKYEK
SYRFHLGQCQWRQPPGKEIYRKSNISVYEVDGKDHKIYCQNLCLLAKLF
YELLDHKTLYFDVEPFVFYILTEVDRQGAHIVGYFSKEKESPDGNNVAC
ILTLPPYQRRGYGKFLIAFSSKLESTVGSPEKPLSDLGKLSYRSYWSWV
LLEILRDFRGTLSIKDLSQMTSITQNDIISTLQSLNMVKYWKGQHVICV
TPKLVEEHLKSAQYKKPPITVDSVCLKWAPPKHKQVKLSKK

### BRPF1:

A codon optimized DNA sequence encoding amino acid residues 1-1214 (Uniprot P55201-1) of human BRPF1 gene was synthesized de novo (Genscript) and ligated into a modified pFastBac vector containing N-terminal Flag affinity tag, followed by a TEV protease cleavage site. The resulting protein sequence is listed below:

(SEQ ID NO. 7)
MASDYKDDDDKGATTENLYFQGSMGVDFDVKTFCHNLRATKPPYECPVE
TCRKVYKSYSGIEYHLYHYDHDNPPPPQQTPLRKHKKKGRQSRPANKQS
PSPSEVSQSPGREVMSYAQAQRMVEVDLHGRVHRISIFDNLDVVSEDEE
APEEAPENGSNKENTETPAATPKSGKHKNKEKRKDSNHHHHHNVSASTT
PKLPEVVYRELEQDTPDAPPRPTSYYRYIEKSAEELDEEVEYDMDEEDY
IWLDIMNERRKTEGVSPIPQEIFEYLMDRLEKESYFESHNKGDPNALVD
EDAVCCICNDGECQNSNVILFCDMCNLAVHQECYGVPYIPEGQWLCRRC
LQSPSRAVDCALCPNKGGAFKQTDDGRWAHVVCALWIPEVCFANTVFLE
PIDSIEHIPPARWKLTCYICKQRGSGACIQCHKANCYTAFHVTCAQQAG
LYMKMEPVRETGANGTSFSVRKTAYCDIHTPPGSARRLPALSHSEGEED
EDEEEDEGKGWSSEKVKKAKAKSRIKMKKARKILAEKRAAAPVVSVPCI
PPHRLSKITNRLTIQRKSQFMQRLHSYWTLKRQSRNGVPLLRRLQTHLQ
SQRNCDQVGRDSEDKNWALKEQLKSWQRLRHDLERARLLVELIRKREKL
KRETIKVQQIAMEMQLTPFLILLRKTLEQLQEKDTGNIFSEPVPLSEVP
DYLDHIKKPMDFFTMKQNLEAYRYLNFDDFEEDFNLIVSNCLKYNAKDT
IFYRAAVRLREQGGAVLRQARRQAEKMGIDFETGMHIPHSLAGDEATHH
TEDAAEEERLVLLENQKHLPVEEQLKLLLERLDEVNASKQSVGRSRRAK
MIKKEMTALRRKLAHQRETGRDGPERHGPSSRGSLTPHPAACDKDGQTD
SAAEESSSQETSKGLGPNMSSTPAHEVGRRTSVLFSKKNPKTAGPPKRP
GRPPKNRESQMTPSHGGSPVGPPQLPIMSSLRQRKRGRSPRPSSSSDSD
SDKSTEDPPMDLPANGFSGGNQPVKKSFLVYRNDCSLPRSSSDSESSSS
SSSSAASDRTSTTPSKQGRGKPSFSRGTFPEDSSEDTSGTENEAYSVGT
GRGVGHSMVRKSLGRGAGWLSEDEDSPLDALDLVWAKCRGYPSYPALII
DPKMPREGMFHHGVPIPVPPLEVLKLGEQMTQEAREHLYLVLFFDNKRT
WQWLPRTKLVPLGVNQDLDKEKMLEGRKSNIRKSVQIAYHRALQHRSKV
QGEQSSETSDSD

### BRPF2:

A codon optimized DNA sequence encoding amino acid residues 2-1058 (Uniprot O95696-1) of human BRPF2 gene was synthesized de novo (Genscript) and ligated into a modified pFastBac vector containing N-terminal Flag affinity tag, followed by a TEV protease cleavage site. The resulting protein sequence is listed below:

(SEQ ID NO. 8)
MASDYKDDDDKGATTENLYFQGSRRKGRCHRGSAARHPSSPCSVKHSPT
RETLTYAQAQRMVEIEIEGRLHRISIFDPLEIILEDDLTAQEMSECNSN
KENSERPPVCLRTKRHKNNRVKKKNEALPSAHGTPASASALPEPKVRIV
EYSPPSAPRRPPVYYKFIEKSAEELDNEVEYDMDEEDYAWLEIVNEKRK
GDCVPAVSQSMFEFLMDRFEKESHCENQKQGEQQSLIDEDAVCCICMDG
ECQNSNVILFCDMCNLAVHQECYGVPYIPEGQWLCRHCLQSRARPADCV
LCPNKGGAFKKTDDDRWGHVVCALWIPEVGFANTVFIEPIDGVRNIPPA
RWKLTCYLCKQKGVGACIQCHKANCYTAFHVTCAQKAGLYMKMEPVKEL
TGGGTTFSVRKTAYCDVHTPPGCTRRPLNIYGDVEMKNGVCRKESSVKT
VRSTSKVRKKAKKAKKALAEPCAVLPTVCAPYIPPQRLNRIANQVAIQR
KKQFVERAHSYWLLKRLSRNGAPLLRRLQSSLQSQRSSQQRENDEEMKA
AKEKLKYWQRLRHDLERARLLIELLRKREKLKREQVKVEQVAMELRLTP
LTVLLRSVLDQLQDKDPARIFAQPVSLKEVPDYLDHIKHPMDFATMRKR
LEAQGYKNLHEFEEDFDLIIDNCMKYNARDTVFYRAAVRLRDQGGVVLR
QARREVDSIGLEEASGMHLPERPAAAPRRPFSWEDVDRLLDPANRAHLG
LEEQLRELLDMLDLTCAMKSSGSRSKRAKLLKKEIALLRNKLSQQHSQP
LPTGPGLEGFEEDGAALGPEAGEEVLPRLETLLQPRKRSRSTCGDSEVE
EESPGKRLDAGLTNGFGGARSEQEPGGGLGRKATPRRRCASESSISSSN
SPLCDSSFNAPKCGRGKPALVRRHTLEDRSELISCIENGNYAKAARIAA
EVGQSSMWISTDAAASVLEPLKVVWAKCSGYPSYPALIIDPKMPRVPGH
HNGVTIPAPPLDVLKIGEHMQTKSDEKLFLVLFFDNKRSWQWLPKSKMV
PLGIDETIDKLKMMEGRNSSIRKAVRIAFDRAMNHLSRVHGEPTSDLSD
ID

### Jade1:

A codon optimized DNA sequence encoding amino acid residues 1-842 (Uniprot Q6IE81-1) of human Jade1 gene was synthesized de novo (Genscript) and ligated into a modified pFastBac vector containing N-terminal Flag affinity tag, followed by a TEV protease cleavage site. The resulting protein sequence is listed below:

(SEQ ID NO. 9)
MASDYKDDDDKGATTENLYFQGSMKRGRLPSSSEDSDDNGSLSTTWSQN
SRSQHRRSSCSRHEDRKPSEVFRTDLITAMKLHDSYQLNPDEYYVLADP
WRQEWEKGVQVPVSPGTIPQPVARVVSEEKSLMFIRPKKYIVSSGSEPP
ELGYVDIRTLADSVCRYDLNDMDAAWLELTNEEFKEMGMPELDEYTMER
VLEEFEQRCYDNMNHAIETEEGLGIEYDEDVVCDVCQSPDGEDGNEMVF
CDKCNICVHQACYGILKVPEGSWLCRTCALGVQPKCLLCPKKGGAMKPT
RSGTKWVHVSCALWIPEVSIGSPEKMEPITKVSHIPSSRWALVCSLCNE
KFGASIQCSVKNCRTAFHVTCAFDRGLEMKTILAENDEVKFKSYCPKHS
SHRKPEESLGKGAAQENGAPECSPRNPLEPFASLEQNREEAHRVSVRKQ
KLQQLEDEFYTFVNLLDVARALRLPEEVVDFLYQYWKLKRKVNFNKPLI
TPKKDEEDNLAKREQDVLFRRLQLFTHLRQDLERVRNLTYMVTRREKIK
RSVCKVQEQIFNLYTKLLEQERVSGVPSSCSSSSLENMLLFNSPSVGPD
APKIEDLKWHSAFFRKQMGTSLVHSLKKPHKRDPLQNSPGSEGKTLLKQ
PDLCGRREGMVVPESFLGLEKTFAEARLISAQQKNGVVMPDHGKRRDNR
FHCDLIKGDLKDKSFKQSHKPLRSTDVSQRHLDNTRAATSPGVGQSAPG
TRKEIVPKCNGSLIKVNYNQTAVKVPTTPASPVKNWGGFRIPKKGERQQ
QGEAHDGACHQHSDYPYLGLGRVPAKERAKSKLKSDNENDGYVPDVEMS
DSESEASEKKCIHTSSTISRRTDIIRRSILAS

### ING5:

A codon optimized DNA sequence encoding amino acid residues 1-240 (Uniprot Q8WYH8-1) of human ING5 gene was synthesized de novo (Genscript) and ligated into a modified pFastBac vector. The resulting protein sequence is listed below:

(SEQ ID NO. 10)
MATAMYLEHYLDSIENLPCELQRNFQLMRELDQRTEDKKAEIDILAAEY
ISTVKTLSPDQRVERLQKIQNAYSKCKEYSDDKVQLAMQTYEMVDKHIR
RLDADLARFEADLKDKMEGSDFESSGGRGLKKGRGQKEKRGSRGRGRRT
SEEDTPKKKKHKGGSEFTDTILSVHPSDVLDMPVDPNEPTYCLCHQVSY
GEMIGCDNPDCPIEWFHFACVDLTTKPKGKWFCPRCVQEKRKKK

MEAF6: A codon optimized DNA sequence encoding amino acid residues 1-201 (Uniprot Q9HAF1-3) of human MEAF6 gene was synthesized de novo (Genscript) and ligated into a modified pFastBac vector. The resulting protein sequence is listed below:

(SEQ ID NO. 11)
MHNKAAPPQIPDTRRELAELVKRKQELAETLANLERQIYAFEGSYLEDT
QMYGNIIRGWDRYLTNQKNSNSKNDRRNRKFKEAERLFSKSSVTSAAAV
SALAGVQDQLIEKREPGSGTESDTSPDFHNQENEPSQEDPEDLDGSVQG
VKPQKAASSTSSGSHHSSHKKRKNKNRHSPSGMFDYDFEIDLKLNKKPR
ADY

Protein Expression: To produce recombinantly expressed KAT5, KAT6A, and KAT8 monomers, a 2-16 liter culture of Spodoptera frugiperda (Sf21) insect cells at a density of 2e6/mL was infected with corresponding high-titer stock of recombinant baculovirus generated from the above plasmids, and allowed to grow in shake flasks for 72 hours at 27° C. To produce recombinantly expressed KAT7:ING5:MEAF6:BRPF1/BRPF2/ or Jade1 heterotetramer, a 2-16 liter culture of Sf21 insect cells at a density of 2e6/mL was infected with equal volumes of corresponding high titer stocks of recombinant baculovirus generated from the above plasmids, and allowed to grow in shake flasks for 72 hours at 27° C. Cell pellets were harvested by centrifugation, and frozen at −80° C. for at least 24 hours prior to purification.

Protein Purification: All purification steps were performed at +4° C. Cells were resuspended in lysis buffer containing 50 mM Tris pH 8.0, 250 mM NaCl, 0.25 mM TCEP, 1/75 mL Roche EDTA-free protease inhibitor tablets, 10 uM leupeptin, and 10 uM E-64 protease inhibitor, then clarified by centrifugation. KAT5 and KAT8 target proteins were purified using nickel ProBond resin (Life Technologies). N-terminal polyhistidine tag was subsequently cleaved off using TEV protease, and protein sample passed over nickel Probond resin again, retaining flow-through fractions. Monomeric KAT6A, and heterotetrameric KAT7:ING5:MEAF6:BRPF1/BRPF2/ and Jade1 protein complexes were purified using anti-Flag resin (Pierce). All protein samples were concentrated and passed over a gel filtration chromatography column (SRT-500, Sepax Technologies), previously equilibrated in 25 mM Hepes pH 7.5, 250 mM NaCl, and 0.25 mM TCEP. Peak fractions were pooled, concentrated to 1-2 mg/mL, flash frozen and stored at −80° C. until further use.

### Biological Assays

### KAT6A Assay Protocol:

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H3 1-21 Biotin labeled peptide [CPC Scientific] and AcCoA [Sigma Inc] in assay buffer to 2×
   3. Dilute KAT6A Full length enzyme [Pfizer Inc] to 2× in assay buffer
   4. Dilute Formic Acid [Sigma Inc] to 5% in Milli-Q H₂O
C. Enzyme reaction
   1. Final reaction conditions are 2.5 nM KAT6A Full length enzyme, 25 μM AcCoA, 2 μM H3 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT6A enzyme mix to the assay plate
   4. Add 10 μL of H3 1-21 peptide and AcCoA mix to the assay plate
   5. Terminate the reaction after 20 mins with the addition of 2 μL of 5% Formic Acid
   6. Neutralize the terminated acidified reaction for 30 mins with 2.0 L of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2723 [Substrate+H]⁺ and 2765 [Product+H]⁺ with a ±1 Da tolerance, respectively
D. Data analysis
   1. Fit the data to the Morrison equation for tight binding competitive inhibition using ActivityBase software

### KAT7 BRPF1 Assay Protocol

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H3 1-21 Biotin labeled peptide [CPC Scientific] and AcCoA [Sigma Inc] in assay buffer to 2×
   3. Dilute KAT7+BRPF1+EAF6+ING5 4 protein complex [Pfizer Inc] to 2× in assay buffer
   4. Dilute Formic Acid [Sigma Inc] to 5% in Milli-Q H₂O
C. Enzyme reaction
   1. Final reaction conditions are 2 nM KAT7+BRPF1+EAF6+ING54-protein complex, 1 μM AcCoA, 2 μM H3 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT7 enzyme mix to the assay plate
   4. Add 10 μL of H3 1-21 peptide and AcCoA mix to the assay plate
   5. Terminate the reaction after 30 mins with the addition of 2 μL of 5% Formic Acid
   6. Neutralize the terminated acidified reaction for 30 mins with 2.0 μL of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2723 [Substrate+H]+ and 2765 [Product+H]+ with a ±1 Da tolerance, respectively
D. Data analysis
   1. Fit the data to the Morrison equation for tight binding competitive inhibition using ActivityBase software

### KAT7 BRPF2 Assay Protocol

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H3 1-21 Biotin labeled peptide [CPC Scientific] and AcCoA [Sigma Inc] in assay buffer to 2×
   3. Dilute KAT7+BRPF2+EAF6+ING5 4 protein complex [Pfizer Inc] to 2× in assay buffer
   4. Dilute Formic Acid [Sigma Inc] to 5% in Milli-Q H₂O
C. Enzyme reaction
   1. Final reaction conditions are 2 nM KAT7+BRPF2+EAF6+ING5 4-protein complex, 1 μM AcCoA, 2 μM H3 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT7 enzyme mix to the assay plate
   4. Add 10 μL of H3 1-21 peptide and AcCoA mix to the assay plate
   5. Terminate the reaction after 30 mins with the addition of 2 μL of 5% Formic Acid
   6. Neutralize the terminated acidified reaction for 30 mins with 2.0 μL of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2723 [Substrate+H]+ and 2765 [Product+H]+ with a ±1 Da tolerance, respectively
D. Data analysis
   1. Fit the data to the Morrison equation for tight binding competitive inhibition using ActivityBase software

### KAT7 JADE1 Assay Protocol

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H3 1-21 Biotin labeled peptide [CPC Scientific] and AcCoA [Sigma Inc] in assay buffer to 2×
   3. Dilute KAT7+JADE1+EAF6+ING5 4 protein complex [Pfizer Inc] to 2× in assay buffer
   4. Dilute Formic Acid [Sigma Inc] to 5% in Milli-Q H₂O
C. Enzyme reaction
   1. Final reaction conditions are 4 nM KAT7+JADE1+EAF6+ING5 4-protein complex, 1 μM AcCoA, 2 μM H3 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT7 enzyme mix to the assay plate
   4. Add 10 μL of H3 1-21 peptide and AcCoA mix to the assay plate
   5. Terminate the reaction after 30 mins with the addition of 2 μL of 5% Formic Acid
   6. Neutralize the terminated acidified reaction for 30 mins with 2.0 μL of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2723 [Substrate+H]+ and 2765 [Product+H]+ with a +1 Da tolerance, respectively
D. Data analysis
   1. Fit the data to the Morrison equation for tight binding competitive inhibition using ActivityBase software

### KAT8 Assay Protocol

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H3 1-21 Biotin labeled peptide [CPC Scientific] and AcCoA [Sigma Inc] in assay buffer to 2×
   3. Dilute KAT8 Full length enzyme [Pfizer Inc] to 2× in assay buffer
   4. Dilute Formic Acid [Sigma Inc] to 5% in Milli-Q H₂O
C. Enzyme reaction
   1. Final reaction conditions are 30 nM KAT8 Full length enzyme, 1 μM AcCoA, 2 μM H3 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT8 enzyme mix to the assay plate
   4. Add 10 μL of H3 1-21 peptide and AcCoA mix to the assay plate
   5. Terminate the reaction after 60 mins with the addition of 2 μL of 5% Formic Acid
   6. Neutralize the terminated acidified reaction for 30 mins with 2.0 μL of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2723 [Substrate+H]+ and 2765 [Product+H]+ with a +1 Da tolerance, respectively
D. Data analysis
   1. Fit the data to the Morrison equation for tight binding competitive inhibition using ActivityBase software

### KAT5 Assay Protocol

A. Compound preparation
   1. Prepare 10 mM stock solutions in 100% DMSO [Sigma Inc] from solid material
   2. Serial dilute 10 mM compound stocks either 2 or 3-fold in 100% DMSO to generate compounds for 11 point dose response
B. Reagent preparation
   1. Prepare 1× assay buffer containing 10 mM Tris pH [Sigma Inc] 8.0, 2.5 mM NaCl [Sigma Inc], 0.5 mM EDTA [Sigma Inc], 0.005% BSG [Sigma Inc] and 0.002% Tween-20 [Sigma Inc]
   2. Dilute H4 1-21 Biotin labeled peptide [CPC Scientific] and AcCoA [Sigma Inc] in assay buffer to 2×
   3. Dilute KAT5 Full length enzyme [Pfizer Inc] to 2× in assay buffer
   4. Dilute Formic Acid [Sigma Inc] to 5% in Milli-Q H₂O
C. Enzyme reaction
   1. Final reaction conditions are 12.5 nM KAT5 Full length enzyme, 1 μM AcCoA, 2 μM H4 1-21 peptide, in a 20 μL reaction volume.
   2. Add 200 nL of diluted compound to the assay plate (384-well microtiter V-bottom polypropylene plates [Greiner Inc]) or 200 nL of DMSO or 200 nL reference inhibitor for the control wells
   3. Add 10 μL of KAT5 enzyme mix to the assay plate
   4. Add 10 μL of H4 1-21 peptide and AcCoA mix to the assay plate
   5. Terminate the reaction after 60 mins with the addition of 2 μL of 5% Formic Acid
   6. Neutralize the terminated acidified reaction for 30 mins with 2.0 μL of 10% Sodium Bicarbonate (1% final) [Sigma Inc]
   7. Transfer 2.6 μL of the neutralized reaction to a 384-well SAMDI plate [SAMDI Tech] surface containing the proprietary Biotin-NeutrAvidin monolayer (Mrksich M. Mass spectrometry of self-assembled monolayers: a new tool for molecular surface science. ACS Nano. 2008 January; 2 (1): 7-18. doi: 10.1021/nn7004156. PMID: 19206542; PMCID: PMC2600870 and Michael D. Scholle, Patrick T. O'Kane, Sandra Dib, Zachary A. Gurard-Levin, Label-free duplex SAMDI-MS screen reveals novel SARS-COV-2 3CLpro inhibitors, Antiviral Research, Volume 200, 2022, 105279, ISSN 0166-3542.)
   8. Immobilize the reactions on the SAMDI plate surface in a humidified chamber for 45 mins at rt
   9. Wash the SAMDI plate surface 5 times using purified Milli-Q H₂O and allow to dry completely with pressurized air using air blades
   10. Apply α-Cyano-4-hydroxycinnamic acid [Sigma Inc] matrix using SAMDI proprietary method
   11. Measure enzyme activity using a MALDI TOF/TOF System [AB Sciex] and determine the area under the curve for the substrate and product peaks at MW 2561 [Substrate+H]+ and 2603 [Product+H]+ with a +1 Da tolerance, respectively
D. Data analysis
   1. Fit the data to the Morrison equation for tight binding competitive inhibition using ActivityBase software

### Data Processing and Analysis

Area under the curve (AUC) for both substrate and product peaks was determined for KAT5 at M.W. 2561 [Substrate+H]⁺ and 2603 [Product+H]⁺ with a +/−1 Da tolerance, respectively. Area under the curve (AUC) for both substrate and product peaks was determined for KAT6A, KAT7 and KAT8 at M.W. 2723 [Substrate+H]⁺ and 2765 [Product+H]⁺ with a +/−1 Da tolerance, respectively. Percent conversion to product was calculated by: AUC_{Product}/(AUC_{Substrate}+AUC_{Product}). IC50 values were determined by fitting the % conversion at each inhibitor concentration to the 4-parameter IC50 equation using Pfizer proprietary curve fitting software. Ki values were determined by fitting the % conversion at each inhibitor concentration to the Morrison equation for tightbinding competitive inhibitors using Pfizer proprietary curve fitting software.

KAT6A, KAT7 BRPF1, KAT7 BRPF2, KAT7 JADE1, KAT8, and KAT5 Ki's are provided in Table 25 below.

TABLE 25

| | | | KAT7 | | KAT7 | | KAT7 | | | | | |
|---|---|---|---|---|---|---|---|---|---|---|---|---|
| | KAT6A Ki | | BRPF1 Ki | | BRPF2 Ki | | JADE1 Ki | | KAT8 Ki | | KAT5 Ki | |
| | (nM) | | (nM) | | (nM) | | (nM) | | (nM) | | (nM) | |
| Example | (replicate | | (replicate | | (replicate | | (replicate | | (replicate | | (replicate | |
| Number | number) | | number) | | number) | | number) | | number) | | number) | |
| 001 | 0.4 | (2) | 37.7 | (2) | 37.0 | (1) | 130.9 | (4) | 455.4 | (1) | 440.6 | (1) |
| 002 | 1.1 | (11) | 3.5 | (7) | 4.8 | (6) | 19.5 | (25) | 2272.9 | (13) | 305.1 | (12) |
| 003 | 0.5 | (1) | 40.6 | (2) | 43.6 | (2) | 105.2 | (7) | 861.2 | (6) | 138.0 | (8) |
| 004 | 0.7 | (1) | 67.2 | (3) | 57.2 | (3) | 250.9 | (7) | 1230.5 | (2) | 367.3 | (2) |
| 005 | 0.5 | (1) | N/D | | N/D | | N/D | | 289.7 | (2) | 13.6 | (3) |
| 006 | 0.8 | (1) | 47.1 | (2) | 50.5 | (2) | 147.5 | (3) | >13041.5 | (1) | 300.1 | (1) |
| 007 | 0.6 | (3) | N/D | | N/D | | 4.2 | (2) | 456.8 | (3) | 53.6 | (3) |
| 008 | 1.2 | (1) | N/D | | N/D | | 12.8 | (1) | 1615.3 | (1) | 46.9 | (1) |
| 009 | 0.8 | (1) | 51.8 | (2) | 90.7 | (1) | 202.4 | (4) | 1193.8 | (2) | 642.4 | (3) |
| 010 | 3.1 | (2) | N/D | | N/D | | 726.8 | (1) | 11470.4 | (1) | 11838.0 | (1) |
| 011 | 31.1 | (1) | N/D | | N/D | | >2336.7 | (1) | N/D | | N/D | |
| 012 | 25.6 | (2) | N/D | | N/D | | >2336.7 | (1) | N/D | | N/D | |
| 013 | 25.5 | (1) | N/D | | N/D | | >2336.7 | (1) | N/D | | N/D | |
| 014 | 10.1 | (1) | N/D | | N/D | | >2336.7 | (1) | >16303.4 | (1) | 44938.8 | (1) |
| 015 | 1.2 | (1) | 67.8 | (2) | 100.3 | (1) | 256.2 | (4) | 1317.1 | (1) | 1332.7 | (1) |
| 016 | 0.2 | (1) | N/D | | N/D | | 701.8 | (1) | 2247.6 | (1) | 1651.2 | (1) |
| 017 | 1.9 | (2) | N/D | | N/D | | 1226.2 | (1) | N/D | | N/D | |
| 018 | 1.0 | (1) | N/D | | N/D | | 244.9 | (1) | 5960.2 | (1) | 5424.4 | (1) |
| 019 | 30.8 | (1) | N/D | | N/D | | 515.6 | (1) | >16303.4 | (1) | 15683.6 | (1) |
| 020 | 5.3 | (1) | N/D | | N/D | | 1537.3 | (1) | 14551.4 | (1) | 9380.3 | (1) |
| 021 | 7.9 | (1) | N/D | | N/D | | 625.0 | (1) | N/D | | N/D | |
| 022 | 0.9 | (1) | 108.5 | (6) | 186.0 | (5) | 300.3 | (8) | 1695.6 | (2) | 488.0 | (2) |
| 023 | 0.9 | (1) | 36.7 | (2) | 95.2 | (2) | 211.7 | (5) | 1057.4 | (3) | 418.1 | (3) |
| 024 | 0.9 | (1) | 88.5 | (2) | 182.4 | (1) | 284.9 | (4) | 440.5 | (1) | 235.7 | (1) |
| 025 | 0.8 | (1) | N/D | | N/D | | N/D | | 446.5 | (1) | 121.3 | (2) |
| 026 | 0.6 | (1) | 14.7 | (2) | 71.5 | (2) | 206.5 | (5) | 153.7 | (2) | 189.4 | (2) |
| 027 | 1.3 | (1) | 74.7 | (2) | 124.0 | (1) | 219.5 | (4) | 893.1 | (1) | 389.7 | (1) |
| 028 | 1.2 | (1) | 97.1 | (6) | 141.0 | (5) | 289.8 | (8) | 3107.5 | (1) | 630.1 | (1) |
| 029 | 1.0 | (1) | 40.5 | (4) | 49.1 | (3) | 181.5 | (5) | 1378.3 | (2) | 594.3 | (2) |
| 030 | 0.5 | (1) | N/D | | 26.0 | (1) | 247.7 | (2) | 675.5 | (1) | 605.4 | (1) |
| 031 | 0.8 | (1) | 79.9 | (2) | 103.6 | (1) | 325.3 | (4) | 781.4 | (1) | 976.6 | (1) |
| 032 | N/D | | 89.7 | (2) | 79.5 | (2) | 360.9 | (5) | >16302.4 | (1) | 2737.4 | (1) |
| 033 | N/D | | 15.4 | (1) | N/D | | 141.7 | (2) | 732.6 | (1) | 988.1 | (1) |
| 034 | 0.4 | (1) | 11.2 | (1) | N/D | | 51.8 | (2) | 1236.4 | (1) | 364.7 | (1) |
| 035 | 3.0 | (1) | 48.3 | (2) | 155.5 | (2) | 527.3 | (5) | 9114.6 | (1) | 1759.0 | (1) |
| 036 | 1.0 | (1) | 93.0 | (3) | 105.9 | (3) | 500.6 | (3) | 2993.2 | (1) | 1082.3 | (1) |
| 037 | 15.9 | (1) | N/D | | N/D | | >2336.7 | (1) | N/D | | N/D | |
| 038 | 60.4 | (1) | N/D | | N/D | | >12479.6 | (1) | N/D | | N/D | |
| 039 | 6.1 | (1) | 75.3 | (3) | 98.2 | (3) | 175.4 | (3) | >32606.7 | (1) | 1750.4 | (4) |
| 040 | 3.1 | (1) | 49.1 | (2) | 156.2 | (2) | 526.2 | (5) | >16302.4 | (1) | 4596.8 | (1) |
| 041 | 6.0 | (1) | 25.6 | (2) | 94.2 | (2) | 358.8 | (5) | >16302.4 | (1) | 3387.1 | (1) |
| 042 | 10.7 | (1) | 26.7 | (2) | 30.8 | (2) | 315.8 | (5) | >16302.4 | (1) | 7117.1 | (1) |
| 043 | 55.5 | (1) | N/D | | N/D | | >2336.7 | (1) | N/D | | N/D | |
| 044 | 3.4 | (1) | 9.8 | (2) | 16.2 | (1) | 48.2 | (5) | >16302.4 | (1) | 1002.3 | (2) |
| 045 | 2.3 | (1) | N/D | | N/D | | 804.5 | (1) | N/D | | N/D | |
| 046 | >376.8 | (1) | >16134.1 | (1) | N/D | | >9773.9 | (1) | N/D | | N/D | |
| 047 | 0.8 | (33) | 1.9 | (4) | 2.2 | (4) | 5.0 | (40) | 1220.7 | (13) | 130.6 | (10) |
| 048 | N/D | | N/D | | N/D | | >12479.6 | (1) | N/D | | N/D | |
| 049 | 4.0 | (1) | N/D | | N/D | | >2336.7 | (1) | N/D | | N/D | |
| 050 | 4.5 | (1) | 28.4 | (1) | 22.8 | (1) | 113.4 | (4) | 709.3 | (1) | 634.3 | (1) |
| 051 | >376.8 | (1) | N/D | | N/D | | >4991.2 | (1) | N/D | | N/D | |
| 052 | 73.5 | (1) | N/D | | N/D | | >12479.6 | (1) | N/D | | N/D | |
| 053 | 19.8 | (1) | N/D | | N/D | | 441.0 | (1) | N/D | | N/D | |
| 054 | 2.0 | (1) | N/D | | N/D | | >2336.7 | (1) | N/D | | N/D | |
| 055 | 2.4 | (1) | 198.6 | (2) | 146.8 | (2) | 663.1 | (5) | N/D | | N/D | |
| 056 | 3.0 | (1) | 156.9 | (2) | 512.3 | (1) | 594.2 | (4) | 7215.6 | (1) | 22243.1 | (1) |
| 057 | 6.2 | (1) | N/D | | N/D | | >2336.7 | (1) | N/D | | N/D | |
| 058 | 5.7 | (1) | N/D | | N/D | | >12479.6 | (1) | N/D | | N/D | |
| 059 | N/D | | 580.5 | (1) | N/D | | >2870.3 | (2) | N/D | | N/D | |
| 060 | >376.8 | (1) | N/D | | N/D | | >12479.6 | (1) | N/D | | N/D | |
| 061 | 0.2 | (1) | N/D | | N/D | | 54.7 | (1) | N/D | | N/D | |
| 062 | 4.9 | (1) | 38.1 | (2) | 35.3 | (2) | 190.9 | (3) | 5343.1 | (1) | 750.5 | (1) |
| 063 | 10.2 | (3) | 54.8 | (5) | 76.2 | (4) | 203.1 | (5) | 12972.3 | (2) | 435.7 | (2) |
| 064 | 0.2 | (1) | 145.8 | (2) | 143.8 | (2) | 135.6 | (3) | 550.0 | (1) | 48.1 | (1) |
| 065 | 8.6 | (1) | 57.4 | (5) | 85.4 | (4) | 205.6 | (5) | >13041.5 | (1) | 1284.3 | (1) |
| 066 | 10.7 | (1) | N/D | | N/D | | 130.1 | (2) | >13041.5 | (1) | 870.6 | (1) |
| 067 | 5.7 | (2) | N/D | | N/D | | 83.8 | (2) | >13041.5 | (1) | 615.7 | (1) |
| 068 | 1.2 | (2) | N/D | | N/D | | 16.8 | (2) | 4134.5 | (2) | 39.3 | (2) |
| 069 | >3288.2 | (1) | N/D | | N/D | | >29001.9 | (1) | >13041.5 | (1) | >49998.4 | (1) |
| 070 | 1.9 | (3) | N/D | | N/D | | 16.4 | (3) | 3878.4 | (3) | 279.8 | (3) |
| 071 | 2.6 | (5) | 2.2 | (1) | N/D | | 20.9 | (6) | 4050.5 | (6) | 591.0 | (6) |
| 072 | 4.6 | (3) | 1.1 | (1) | N/D | | 21.5 | (3) | >6967.0 | (3) | 1009.2 | (3) |
| 073 | 0.7 | (1) | N/D | | N/D | | 31.3 | (2) | 4327.6 | (2) | 286.8 | (2) |
| 074 | 5.2 | (2) | N/D | | N/D | | 482.7 | (3) | >13041.5 | (1) | 2162.0 | (1) |
| 075 | 22.3 | (2) | N/D | | N/D | | 501.4 | (2) | >11198.3 | (2) | 2214.7 | (2) |
| 076 | 11.1 | (3) | N/D | | N/D | | 92.0 | (3) | >13041.5 | (1) | 1160.7 | (2) |
| 077 | 0.6 | (1) | 76.1 | (2) | 161.5 | (2) | 213.4 | (3) | 2757.0 | (1) | 242.6 | (1) |
| 078 | 2.5 | (1) | 40.2 | (2) | 54.4 | (2) | 108.6 | (3) | 8732.5 | (1) | 353.8 | (1) |
| 079 | 5.1 | (1) | 25.2 | (2) | 26.5 | (2) | 119.0 | (3) | 6572.6 | (1) | 692.3 | (1) |
| 080 | 3.8 | (2) | N/D | | N/D | | 32.3 | (2) | 7758.3 | (2) | 1452.5 | (2) |
| 081 | 3.5 | (2) | N/D | | N/D | | 31.6 | (2) | >13041.5 | (1) | 336.0 | (2) |
| 082 | 0.1 | (1) | 7.5 | (2) | 18.3 | (2) | 31.8 | (3) | 466.6 | (1) | 33.8 | (1) |
| 083 | 2.9 | (1) | N/D | | 7.8 | (2) | 60.1 | (3) | 10355.5 | (1) | 501.9 | (1) |
| 084 | 0.2 | (2) | 12.2 | (2) | 10.1 | (2) | 50.6 | (3) | 2972.1 | (2) | 82.3 | (2) |
| 085 | 4.7 | (2) | N/D | | N/D | | 33.4 | (2) | 3383.0 | (2) | 224.8 | (2) |
| 086 | 8.9 | (2) | N/D | | N/D | | 551.7 | (2) | >13041.5 | (1) | 1279.7 | (2) |
| 087 | 23.5 | (2) | N/D | | N/D | | 1230.2 | (2) | >13041.5 | (1) | 1803.3 | (2) |
| 088 | 27.0 | (2) | N/D | | N/D | | 378.4 | (2) | >13041.5 | (1) | 6017.0 | (1) |
| 089 | 3.0 | (3) | N/D | | N/D | | 39.3 | (3) | >12176.4 | (3) | 167.7 | (3) |
| 090 | 3.4 | (4) | N/D | | N/D | | 62.4 | (4) | >12983.7 | (4) | 250.7 | (4) |
| 091 | 2.3 | (2) | N/D | | N/D | | 801.9 | (4) | N/D | | N/D | |
| 092 | 1.3 | (1) | N/D | | N/D | | 219.4 | (2) | 2096.3 | (1) | 867.0 | (1) |
| 093 | 31.2 | (1) | N/D | | N/D | | 729.1 | (2) | N/D | | N/D | |
| 094 | 6.3 | (2) | N/D | | N/D | | 58.1 | (2) | 5739.0 | (2) | 192.8 | (2) |
| 095 | 0.1 | (3) | N/D | | N/D | | 0.3 | (3) | 77.0 | (2) | 18.1 | (3) |
| 096 | 7.3 | (3) | N/D | | 13.6 | (1) | 103.1 | (6) | >13041.5 | (1) | 2054.5 | (1) |
| 097 | 9.8 | (2) | N/D | | N/D | | 57.7 | (2) | >13041.5 | (1) | 794.7 | (2) |
| 098 | 5.7 | (2) | N/D | | N/D | | 74.6 | (2) | >13041.5 | (1) | 3785.8 | (2) |
| 099 | 0.5 | (1) | N/D | | 5.5 | (1) | 82.6 | (2) | 4839.0 | (1) | 388.1 | (1) |
| 100 | 21.4 | (2) | N/D | | N/D | | 86.6 | (2) | >13041.5 | (1) | 1063.3 | (2) |
| 101 | 5.3 | (2) | N/D | | N/D | | 50.7 | (2) | 10310.1 | (2) | 1233.3 | (2) |
| 102 | 1.9 | (2) | N/D | | N/D | | 1443.6 | (3) | >19512.4 | (1) | 5968.4 | (2) |
| 103 | 2.8 | (2) | N/D | | N/D | | 47.4 | (2) | 6009.7 | (2) | 253.5 | (2) |
| 104 | 5.6 | (2) | N/D | | N/D | | 139.0 | (2) | >10942.3 | (2) | 1041.9 | (2) |
| 105 | 9.7 | (3) | N/D | | N/D | | 413.6 | (3) | >12699.0 | (3) | 4119.6 | (3) |
| 106 | 11.7 | (1) | N/D | | N/D | | 409.5 | (4) | 6426.7 | (2) | 1990.1 | (2) |
| 107 | 131.8 | (2) | N/D | | N/D | | 6536.1 | (2) | >13041.5 | (1) | 10380.5 | (2) |
| 108 | 202.5 | (2) | N/D | | N/D | | 3421.1 | (2) | >13041.5 | (1) | 45005.7 | (2) |
| 109 | 0.8 | (4) | 5.0 | (1) | N/D | | 18.5 | (4) | 2934.3 | (3) | 325.6 | (3) |
| 110 | 44.1 | (2) | N/D | | N/D | | 2816.0 | (2) | >13041.5 | (1) | 6605.2 | (2) |
| 111 | 0.8 | (4) | N/D | | N/D | | 400.1 | (4) | >13041.5 | (1) | 1128.0 | (2) |
| 112 | 17.6 | (2) | N/D | | N/D | | 238.4 | (2) | >13041.5 | (1) | 689.1 | (2) |
| 113 | 348.5 | (2) | N/D | | N/D | | 9091.2 | (1) | >13041.5 | (1) | 49998.4 | (1) |
| 114 | 1.3 | (3) | 2.1 | (1) | N/D | | 22.0 | (3) | 3364.1 | (3) | 312.3 | (3) |
| 115 | 0.1 | (1) | N/D | | N/D | | 32.2 | (2) | 8916.2 | (1) | 100.6 | (2) |
| 116 | 11.8 | (2) | N/D | | N/D | | 98.0 | (2) | 7897.3 | (1) | 545.5 | (1) |
| 117 | 5.3 | (3) | N/D | | N/D | | 25.1 | (3) | 10038.9 | (2) | 359.8 | (2) |
| 118 | 0.9 | (2) | N/D | | N/D | | 8.1 | (2) | 730.7 | (2) | 66.1 | (2) |
| 119 | 0.2 | (3) | N/D | | N/D | | 3.6 | (2) | 156.1 | (3) | 19.4 | (3) |
| 120 | 0.8 | (2) | N/D | | N/D | | 10.8 | (2) | 640.5 | (1) | 236.2 | (1) |
| 121 | 0.7 | (3) | 2.1 | (1) | N/D | | 8.9 | (3) | 1124.2 | (3) | 106.5 | (3) |
| 122 | 0.3 | (5) | 1.2 | (1) | N/D | | 3.0 | (5) | 395.2 | (3) | 45.3 | (3) |
| 123 | 0.8 | (2) | N/D | | N/D | | 10.4 | (2) | 188.9 | (1) | 245.8 | (1) |
| 124 | 51.3 | (1) | N/D | | N/D | | 47.9 | (1) | 8220.6 | (1) | 4570.4 | (1) |
| 125 | 48.6 | (1) | N/D | | N/D | | 180.0 | (1) | >13041.5 | (1) | 5088.0 | (1) |
| 126 | 52.1 | (1) | N/D | | N/D | | 236.7 | (1) | >13041.5 | (1) | 5037.4 | (1) |
| 127 | 32.8 | (1) | N/D | | N/D | | 175.2 | (1) | >13041.5 | (1) | 3936.1 | (1) |
| 128 | 38.1 | (1) | N/D | | N/D | | 211.6 | (1) | >13041.5 | (1) | 7833.4 | (1) |
| 129 | 56.8 | (1) | N/D | | N/D | | 244.1 | (1) | >13041.5 | (1) | 5629.2 | (1) |
| 130 | 13.0 | (1) | N/D | | N/D | | 85.2 | (1) | 7516.7 | (1) | 2010.8 | (1) |
| 131 | 8.2 | (1) | N/D | | N/D | | 54.8 | (1) | >13041.5 | (1) | 852.4 | (1) |
| 132 | 82.7 | (2) | N/D | | N/D | | 410.4 | (2) | >13041.5 | (1) | 5520.1 | (1) |
| 133 | 78.7 | (1) | N/D | | N/D | | 320.7 | (1) | >13041.5 | (1) | 6205.1 | (1) |
| 134 | 34.3 | (1) | N/D | | N/D | | 185.2 | (1) | 9798.1 | (1) | 4547.9 | (1) |
| 135 | 52.6 | (1) | N/D | | N/D | | 156.2 | (1) | >13041.5 | (1) | 3080.0 | (1) |
| 136 | 16.3 | (2) | N/D | | N/D | | 92.7 | (2) | >13041.5 | (1) | 2941.5 | (1) |
| 137 | 12.9 | (2) | N/D | | N/D | | 9.7 | (2) | >13041.5 | (1) | 1652.9 | (1) |
| 138 | 1.2 | (1) | N/D | | 5.6 | (1) | 40.6 | (2) | >13041.5 | (1) | 195.2 | (1) |
| 139 | 0.1 | (2) | N/D | | N/D | | 127.7 | (3) | N/D | | N/D | |
| 140 | 1.5 | (4) | N/D | | N/D | | 11.4 | (4) | 911.2 | (1) | 156.3 | (1) |
| 141 | 1.0 | (5) | N/D | | N/D | | 6.9 | (5) | 761.9 | (1) | 133.5 | (1) |
| 142 | 0.7 | (5) | N/D | | N/D | | 6.7 | (6) | N/D | | N/D | |
| 143 | 1.1 | (2) | N/D | | N/D | | 9.6 | (2) | 436.4 | (1) | 44.3 | (1) |
| 144 | 1.8 | (3) | N/D | | N/D | | 10.2 | (3) | 2779.1 | (1) | N/D | |
| 145 | 1.8 | (6) | N/D | | N/D | | 9.2 | (5) | 1514.6 | (1) | N/D | |
| 146 | 0.9 | (18) | 2.4 | (1) | 1.7 | (6) | 3.2 | (19) | 865.3 | (10) | 31.4 | (8) |

| 147 | 1.6 | (2) | N/D | | N/D | 12.1 | (2) | N/D | | N/D | |
|---|---|---|---|---|---|---|---|---|---|---|---|
| 148 | 1.1 | (2) | N/D | | N/D | 3.6 | (3) | 575.3 | (1) | 78.1 | (1) |
| 149 | 0.6 | (2) | N/D | | N/D | 5.3 | (2) | 19022.3 | (1) | N/D | |
| 150 | 0.8 | (2) | N/D | | N/D | 6.3 | (2) | 10292.1 | (1) | N/D | |
| 151 | 0.6 | (1) | N/D | | N/D | 4.8 | (1) | N/D | | N/D | |
| 152 | 0.7 | (2) | N/D | | N/D | 4.1 | (2) | 1159.5 | (1) | 105.5 | (1) |
| 153 | 0.8 | (2) | N/D | | N/D | 5.7 | (2) | 571.0 | (1) | 17.4 | (1) |
| 154 | 2.0 | (2) | 7.8 | (1) | N/D | 15.3 | (2) | 2137.2 | (1) | 88.3 | (1) |

| | | | | | | | | | |
|---|---|---|---|---|---|---|---|---|---|
| 155 | 2.9 | (1) | 14.9 | (1) | N/D | 24.3 | (2) | N/D | N/D |

| 156 | 0.8 | (2) | N/D | N/D | | 4.0 | (2) | N/D | | N/D | |
|---|---|---|---|---|---|---|---|---|---|---|---|
| 157 | 2.5 | (2) | N/D | N/D | | 11.6 | (2) | N/D | | N/D | |
| 158 | 1.2 | (2) | N/D | N/D | | 8.7 | (2) | N/D | | N/D | |
| 159 | 1.1 | (2) | N/D | N/D | | 7.0 | (2) | N/D | | N/D | |
| 160 | 1.0 | (2) | N/D | N/D | | 6.9 | (2) | N/D | | N/D | |
| 161 | 1.2 | (2) | N/D | N/D | | 5.5 | (2) | N/D | | N/D | |
| 162 | 1.2 | (2) | N/D | N/D | | 8.9 | (2) | N/D | | N/D | |
| 163 | 1.4 | (2) | N/D | N/D | | 8.2 | (2) | N/D | | N/D | |
| 164 | 0.8 | (2) | N/D | N/D | | 4.1 | (2) | N/D | | N/D | |
| 165 | 1.4 | (2) | N/D | N/D | | 5.0 | (2) | N/D | | N/D | |
| 166 | 1.6 | (2) | N/D | N/D | | 8.5 | (2) | N/D | | N/D | |
| 167 | 0.9 | (2) | N/D | N/D | | 4.0 | (2) | N/D | | N/D | |
| 168 | 0.8 | (2) | N/D | N/D | | 2.7 | (2) | N/D | | N/D | |
| 169 | 0.5 | (2) | N/D | N/D | | 3.2 | (2) | N/D | | N/D | |
| 170 | 0.4 | (2) | N/D | N/D | | 1.6 | (2) | N/D | | N/D | |
| 171 | 2.1 | (2) | N/D | N/D | | 8.4 | (2) | N/D | | N/D | |
| 172 | 4.4 | (2) | N/D | N/D | | 34.1 | (2) | N/D | | N/D | |
| 173 | 7.5 | (1) | N/D | N/D | | 113.7 | (1) | N/D | | N/D | |
| 174 | 4.4 | (2) | N/D | N/D | | 27.3 | (2) | N/D | | N/D | |
| 175 | 0.4 | (3) | N/D | N/D | | 1.0 | (3) | N/D | | N/D | |
| 176 | 1.0 | (3) | N/D | N/D | | 2.7 | (3) | N/D | | N/D | |
| 177 | 0.1 | (2) | N/D | N/D | | 0.2 | (2) | N/D | | N/D | |
| 178 | 0.3 | (2) | N/D | N/D | | 1.5 | (2) | N/D | | N/D | |
| 179 | 1.0 | (2) | N/D | N/D | | 8.6 | (2) | N/D | | N/D | |
| 180 | 1.7 | (2) | N/D | N/D | | 6.5 | (2) | 3367.8 | (1) | 94.8 | (1) |
| 181 | 2.0 | (2) | N/D | N/D | | 6.2 | (2) | N/D | | N/D | |
| 182 | 0.8 | (1) | N/D | N/D | | 6.4 | (1) | N/D | | N/D | |
| 183 | 2.1 | (2) | N/D | 2.1 | (1) | 5.7 | (2) | N/D | | N/D | |
| 184 | 1.8 | (2) | N/D | 1.6 | (1) | 4.7 | (2) | 1034.9 | (1) | 60.7 | (1) |
| 185 | 0.3 | (2) | N/D | 0.9 | (1) | 1.8 | (2) | 1215.4 | (1) | 1.0 | (1) |
| 186 | 0.8 | (2) | N/D | 1.5 | (1) | 3.6 | (2) | 589.8 | (2) | 5.8 | (2) |
| 187 | 2.2 | (2) | N/D | N/D | | 13.1 | (2) | N/D | | N/D | |
| 188 | 2.5 | (4) | N/D | 2.9 | (1) | 8.4 | (4) | 3694.4 | (2) | 139.7 | (2) |

| 189 | 3.7 | (4) | N/D | | N/D | 20.8 | (4) | N/D | | N/D | |
|---|---|---|---|---|---|---|---|---|---|---|---|
| 190 | 5.6 | (1) | N/D | | N/D | 54.5 | (1) | N/D | | N/D | |
| 191 | 2.3 | (2) | N/D | | N/D | 15.4 | (2) | N/D | | N/D | |
| 192 | 2.4 | (2) | N/D | | N/D | 22.2 | (2) | N/D | | N/D | |
| 193 | 1.4 | (2) | N/D | | N/D | 12.0 | (2) | N/D | | N/D | |
| 194 | 1.7 | (2) | N/D | | N/D | 6.3 | (2) | N/D | | N/D | |
| 195 | 2.5 | (2) | N/D | | N/D | 18.9 | (2) | N/D | | N/D | |
| 196 | 0.9 | (2) | N/D | | N/D | 4.0 | (2) | N/D | | N/D | |
| 197 | 1.3 | (2) | N/D | | N/D | 7.3 | (2) | N/D | | N/D | |
| 198 | 1.1 | (2) | N/D | | N/D | 5.9 | (2) | N/D | | N/D | |
| 199 | 0.7 | (2) | N/D | | N/D | 5.2 | (2) | N/D | | N/D | |
| 200 | 2.1 | (2) | N/D | | N/D | 17.2 | (2) | N/D | | N/D | |
| 201 | 0.6 | (3) | 3.4 | (1) | N/D | 4.1 | (3) | 2185.7 | (1) | 192.1 | (1) |
| 202 | 0.7 | (2) | N/D | | N/D | 5.2 | (2) | 843.6 | (1) | 25.4 | (1) |
| 203 | 0.4 | (2) | 3.0 | (1) | N/D | 4.3 | (2) | 1224.2 | (1) | 21.7 | (1) |

| 204 | 1.1 | (2) | N/D | N/D | | 4.9 | (2) | N/D | N/D |
|---|---|---|---|---|---|---|---|---|---|
| 205 | 1.4 | (2) | N/D | N/D | | 6.0 | (2) | N/D | N/D |
| 206 | 4.4 | (2) | N/D | N/D | | 28.8 | (2) | N/D | N/D |
| 207 | 2.3 | (2) | N/D | 4.0 | (1) | 11.5 | (2) | N/D | N/D |

| 208 | 2.4 | (1) | N/D | | N/D | 7.6 | (1) | N/D | | N/D | |
|---|---|---|---|---|---|---|---|---|---|---|---|
| 209 | 1.4 | (2) | N/D | | N/D | 7.7 | (2) | 2404.1 | (1) | 106.4 | (1) |
| 210 | 1.2 | (1) | N/D | | N/D | 6.6 | (1) | N/D | | N/D | |
| 211 | 1.1 | (2) | N/D | | N/D | 3.7 | (2) | 1349.1 | (2) | 89.4 | (2) |
| 212 | 0.7 | (2) | N/D | | N/D | 11.1 | (3) | 1406.7 | (2) | 158.6 | (1) |
| 213 | 0.5 | (2) | 1.7 | (1) | N/D | 3.9 | (2) | 1572.9 | (1) | N/D | |
| 214 | 0.7 | (2) | N/D | | N/D | 4.3 | (2) | 1720.5 | (1) | N/D | |
| 215 | 0.4 | (2) | N/D | | N/D | 4.2 | (2) | 993.7 | (1) | N/D | |
| 216 | 0.2 | (2) | N/D | | N/D | 1.6 | (2) | N/D | | N/D | |
| 217 | 0.2 | (2) | N/D | | N/D | 1.9 | (2) | N/D | | N/D | |
| 218 | 0.1 | (2) | N/D | | N/D | 0.4 | (2) | N/D | | N/D | |
| 219 | 0.2 | (2) | N/D | | N/D | 1.9 | (2) | N/D | | N/D | |
| 220 | 0.4 | (2) | N/D | | N/D | 4.0 | (2) | N/D | | N/D | |
| 221 | 0.7 | (3) | N/D | | N/D | 6.2 | (2) | 1489.4 | (1) | N/D | |
| 222 | 0.5 | (2) | 4.0 | (1) | N/D | 4.5 | (2) | 957.8 | (1) | 27.9 | (1) |
| 223 | 0.4 | (2) | N/D | | N/D | 4.0 | (2) | 678.6 | (1) | 25.4 | (1) |
| 224 | 1.5 | (1) | N/D | | N/D | 7.2 | (2) | N/D | | N/D | |
| 225 | 0.6 | (2) | N/D | | N/D | 7.0 | (2) | N/D | | N/D | |
| 226 | 0.3 | (3) | 2.4 | (1) | N/D | 3.0 | (3) | 571.6 | (2) | 11.6 | (2) |

| 227 | 0.4 | (2) | N/D | N/D | | 6.9 | (2) | 3490.3 | (1) | 217.5 | (1) |
|---|---|---|---|---|---|---|---|---|---|---|---|
| 228 | 0.8 | (2) | N/D | N/D | | 6.2 | (2) | N/D | | N/D | |
| 229 | 1.7 | (2) | N/D | N/D | | 6.4 | (2) | N/D | | N/D | |
| 230 | 1.0 | (2) | N/D | N/D | | 2.3 | (2) | 1961.5 | (2) | 158.1 | (2) |
| 231 | 1.1 | (2) | N/D | N/D | | 8.8 | (2) | 858.6 | (1) | N/D | |
| 232 | 0.6 | (2) | N/D | N/D | | 6.4 | (2) | 2615.5 | (1) | N/D | |
| 233 | 1.7 | (2) | N/D | N/D | | 8.9 | (2) | 2003.6 | (1) | N/D | |
| 234 | 1.0 | (2) | N/D | N/D | | 8.5 | (2) | 1678.5 | (1) | N/D | |
| 235 | 0.8 | (1) | N/D | N/D | | 6.0 | (1) | 2360.3 | (1) | N/D | |
| 236 | 0.4 | (2) | N/D | N/D | | 3.5 | (2) | 363.5 | (1) | N/D | |
| 237 | 1.4 | (2) | N/D | N/D | | 15.4 | (2) | N/D | | N/D | |
| 238 | 1.2 | (1) | N/D | N/D | | 12.7 | (1) | N/D | | N/D | |
| 239 | 2.1 | (2) | N/D | N/D | | 14.1 | (2) | N/D | | N/D | |
| 240 | 1.2 | (2) | N/D | N/D | | 7.5 | (2) | N/D | | N/D | |
| 241 | 2.6 | (2) | N/D | N/D | | 75.3 | (3) | N/D | | N/D | |
| 242 | 0.9 | (2) | N/D | N/D | | 9.2 | (3) | 439.1 | (3) | 34.0 | (1) |
| 243 | 1.5 | (2) | N/D | N/D | | 16.9 | (2) | 871.5 | (2) | 167.7 | (1) |
| 244 | 0.2 | (1) | N/D | N/D | | 1.3 | (1) | N/D | | N/D | |
| 245 | 0.7 | (2) | N/D | N/D | | 3.6 | (2) | N/D | | N/D | |
| 246 | 1.0 | (3) | N/D | N/D | | 6.4 | (2) | 1844.9 | (1) | N/D | |
| 247 | 0.5 | (2) | N/D | 1.7 | (1) | 2.7 | (2) | N/D | | N/D | |
| 248 | 1.2 | (2) | N/D | 2.6 | (1) | 5.8 | (2) | N/D | | N/D | |
| 249 | 0.3 | (2) | N/D | 0.3 | (1) | 1.7 | (2) | 104.1 | (1) | 8.7 | (1) |
| 250 | 0.2 | (2) | N/D | 0.9 | (1) | 1.3 | (2) | N/D | | N/D | |
| 251 | 1.0 | (2) | N/D | 2.1 | (1) | 6.2 | (2) | 1147.5 | (2) | 67.0 | (2) |
| 252 | 0.8 | (2) | N/D | N/D | | 5.7 | (2) | N/D | | N/D | |
| 253 | 0.6 | (2) | N/D | N/D | | 5.0 | (2) | N/D | | N/D | |
| 254 | 0.7 | (2) | N/D | 1.2 | (1) | 6.6 | (2) | 1262.5 | (1) | 54.5 | (1) |

| 255 | 0.8 | (2) | N/D | N/D | | 4.2 | (2) | N/D | N/D |
|---|---|---|---|---|---|---|---|---|---|
| 256 | 1.1 | (2) | N/D | 0.8 | (1) | 4.7 | (2) | N/D | N/D |

| 257 | 1.6 | (2) | N/D | N/D | 9.5 | (2) | N/D | | N/D | |
|---|---|---|---|---|---|---|---|---|---|---|
| 258 | 1.0 | (2) | N/D | N/D | 5.9 | (2) | N/D | | N/D | |
| 259 | 1.2 | (2) | N/D | N/D | 6.7 | (2) | 4989.7 | (1) | 85.1 | (1) |

| 260 | 1.0 | (2) | N/D | N/D | 6.7 | (2) | N/D | | N/D |
|---|---|---|---|---|---|---|---|---|---|
| 261 | 1.1 | (2) | N/D | N/D | 6.7 | (2) | N/D | | N/D |
| 262 | 1.0 | (2) | N/D | N/D | 8.1 | (2) | N/D | | N/D |
| 263 | 1.4 | (2) | N/D | N/D | 9.8 | (2) | N/D | | N/D |
| 264 | 2.3 | (2) | N/D | N/D | 17.6 | (2) | N/D | | N/D |
| 265 | 0.3 | (2) | N/D | N/D | 2.3 | (2) | 918.4 | (1) | N/D |
| 266 | 1.6 | (3) | N/D | N/D | 13.6 | (2) | N/D | | N/D |
| 267 | 1.3 | (3) | N/D | N/D | 9.3 | (2) | N/D | | N/D |
| 268 | 1.2 | (2) | N/D | N/D | 8.8 | (2) | N/D | | N/D |
| 269 | 0.8 | (2) | N/D | N/D | 6.1 | (2) | N/D | | N/D |
| 270 | 1.3 | (3) | N/D | N/D | 13.5 | (2) | 1788.3 | (1) | N/D |
| 271 | 1.0 | (2) | N/D | N/D | 7.0 | (2) | 1011.3 | (1) | N/D |
| 272 | 0.7 | (2) | N/D | N/D | 5.0 | (2) | 2647.0 | (1) | N/D |
| 273 | 0.6 | (2) | N/D | N/D | 4.7 | (2) | 2020.9 | (1) | N/D |

| | | | | | | | | |
|---|---|---|---|---|---|---|---|---|
| 274 | 2.8 | (2) | N/D | N/D | 6.9 | (2) | N/D | N/D |
| 275 | 1.1 | (2) | N/D | N/D | 9.7 | (2) | N/D | N/D |
| 276 | 1.3 | (1) | N/D | N/D | 16.8 | (1) | N/D | N/D |

1. A compound of Formula (V): 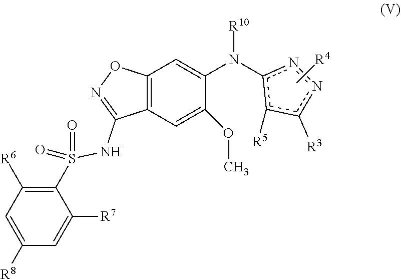
or a pharmaceutically acceptable salt thereof, wherein:
R³ is hydrogen, halogen, C₁-C₄ alkyl, —CH₂OCH₃, methoxy, —C(O)OH, —C(O)OCH₃, C₃-C₅ cycloalkyl, 4-6 membered heterocycloalkyl, or benzyl, wherein the C₁-C₄ alkyl is optionally substituted by —OH or one, two, or three fluorine atoms, the methoxy is optionally substituted by one, two, or three fluorine atoms, and the C₃-C₈ cycloalkyl is optionally substituted by methyl or by one or two fluorine atoms;
R⁴ is hydrogen or C₁-C₃ alkyl optionally substituted by —OH;
R⁵ is hydrogen, halogen, cyano, C₁-C₃ alkyl, or —C(O)NH₂;
R⁶ is hydrogen, halogen, C₁-C₄ alkyl, —O—(C₁-C₄ alkyl), or —O—(C₃-C₅ cycloalkyl), wherein the —O—(C₁-C₄ alkyl) is optionally substituted by one, two, or three fluorine atoms;
R⁷ is hydrogen, C₁-C₄ alkyl, or methoxy;
R⁸ is hydrogen, halogen, cyano, C₁-C₄ alkyl, methoxy, phenyl, —(CHR¹¹)ₙ-4-8 membered heterocycloalkyl, 5-8 membered heteroaryl, 9-10 membered heteroaryl, —C(O)NR¹²R¹³, —N(CH₃)(4-5 membered heterocycloalkyl), or —O-phenyl,
wherein the C₁-C₄ alkyl is optionally substituted by one or two methoxy substituents, —N(R¹⁴)(R¹⁵), or one, two, or three fluorine atoms,
wherein the phenyl is optionally substituted by methoxy,
wherein the —(CHR¹¹)ₙ-4-8 membered heterocycloalkyl is optionally substituted by one, two, or three substituents independently selected from oxo, one or two methyl substituents, ethyl, —CHF₂, —CF₃, —CH₂CHF₂, —CH₂CF₃, methoxy, 4-6 membered heterocycloalkyl, and one or two fluorine atoms,
wherein the 5-8 membered heteroaryl is optionally substituted by oxo, cyano, methyl, —[CH(R¹⁶)]ₚ[N(CH₃)₂], or a 6 membered heterocycloalkyl, which is optionally substituted by methyl,
wherein the 9-10 membered heteroaryl is optionally substituted by methyl,
wherein the —C(O)NR¹²R¹³ is optionally substituted by one or two fluorine atoms, and
wherein the —O-phenyl is optionally substituted by fluoro, methyl, or methoxy;
R¹⁰ is hydrogen or C₁₋₃ alkyl;
R¹¹ is hydrogen or methyl;
R¹² is hydrogen, C₁₋₃ alkyl, —(CH₂)₅NH₂, or —(CH₂)₅NHC(O)-phenyl-(1,2,4,5-tetrazine)-CH₃;
R¹³ is hydrogen or C₁₋₂ alkyl;
wherein when R¹² is C₁₃ alkyl and R¹³ is C₁₋₂ alkyl, R¹² and R¹³ may be taken together with the nitrogen to which they are attached to form a 4-6 membered heterocycloalkyl ring, wherein the 4-6 membered heterocycloalkyl ring is optionally substituted by one or two fluorine atoms;
each R¹⁴ and R¹⁵ is independently methyl or ethyl;
R¹⁶ is hydrogen or methyl;
n is 0 or 1; and
p is 0 or 1,
wherein one or more hydrogen atoms are replaced by deuterium.

2. The compound of claim 1, wherein the compound of Formula (V) is 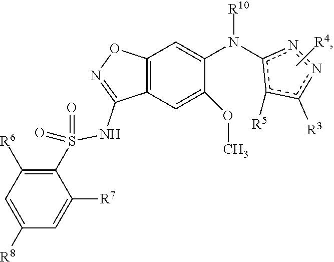
wherein
* is a metabolic site,
further wherein one or more hydrogen atoms of the group consisting of R⁶, R⁷, R⁸ and the metabolic site are replaced by deuterium.

3. The compound of claim 1, wherein R³ is hydrogen, chloro, C₁-C₄ alkyl, —CHF₂, —CF₃, —CF₂CH₃, —CH₂—CF₃, —CH₂OCH₃, methoxy, —O—CHF₂, —C(O)OH, —C(O)OCH₃, bicyclo[1.1.1]pentan-1-yl, cyclopropyl, cyclobutyl, cyclopentyl, piperidinyl, or benzyl, wherein the C₁-C₄ alkyl is optionally substituted by —OH and the cyclopropyl is optionally substituted by methyl or two fluorine atoms.

4. The compound of claim 1, wherein R³ is cyclopropyl.

5. The compound of claim 1, wherein R⁴ is hydrogen.

6. The compound of claim 1, wherein R⁵ is hydrogen.

7. The compound of claim 1, wherein R³ is cyclopropyl, R⁴ is hydrogen, and R⁵ is hydrogen.

8. The compound of claim 1, wherein R⁶ is methoxy and R⁷is methoxy.

9. The compound of claim 1, wherein R⁸ is hydrogen, chloro, fluoro, cyano, methyl, ethyl, propyl, —CHF₂, —CF₃, —CH₂OCH₃, methoxy, phenyl, azetidinyl, oxetanyl, tetrahydrofuranyl, pyrrolidinyl, tetrahydropyranyl, piperidinyl, morpholinyl, imidazolyl, pyrazolyl, oxazolyl, thiazolyl, pyridinyl, pyrazinyl, —C(O)N(CH₃)₂, —C(O)NH(CH₂)₅NH₂, —C(O)NH(CH₂)₅NHC(O)-phenyl-(1,2,4,5-tetrazine)-CH₃ or —O-phenyl, wherein the phenyl is optionally substituted by methoxy, wherein the pyrazolyl, the imidazolyl and the thiazolyl are each independently optionally substituted by methyl, and wherein the —O-phenyl is optionally substituted by methyl, fluoro or methoxy.

10. The compound of claim 1, wherein R¹⁰ is hydrogen.

11. The compound of claim 1, which is selected from the group consisting of 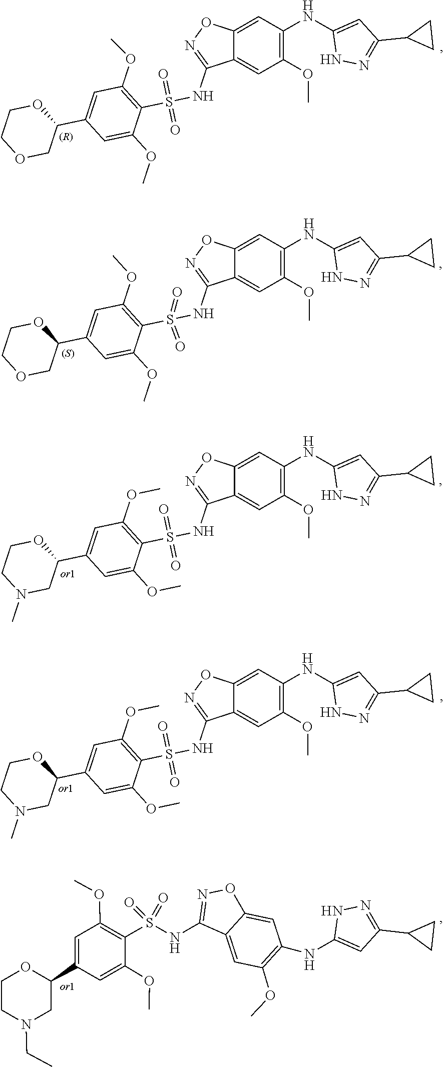 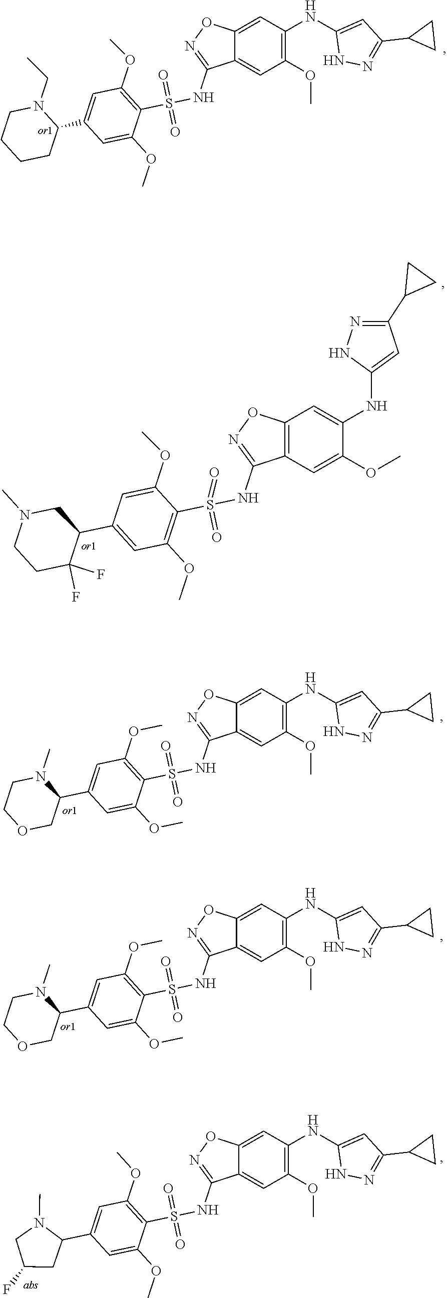 [SMILES: CCN1CCCC[C@H]1c1cc(OC)c(S(=O)(=O)Nc2noc3cc(Nc4cc(C5CC5)n[nH]4)c(OC)cc23)c(OC)c1 ; COc1cc2c(NS(=O)(=O)c3c(OC)cc([C@@H]4CN(C)CCC4(F)F)cc3OC)noc2cc1Nc1cc(C2CC2)n[nH]1 ; COc1cc2c(NS(=O)(=O)c3c(OC)cc([C@H]4COCCN4C)cc3OC)noc2cc1Nc1cc(C2CC2)n[nH]1 ; COc1cc2c(NS(=O)(=O)c3c(OC)cc([C@H]4COCCN4C)cc3OC)noc2cc1Nc1cc(C2CC2)n[nH]1 ; COc1cc2c(NS(=O)(=O)c3c(OC)cc(C4C[C@H](F)CN4C)cc3OC)noc2cc1Nc1cc(C2CC2)n[nH]1] 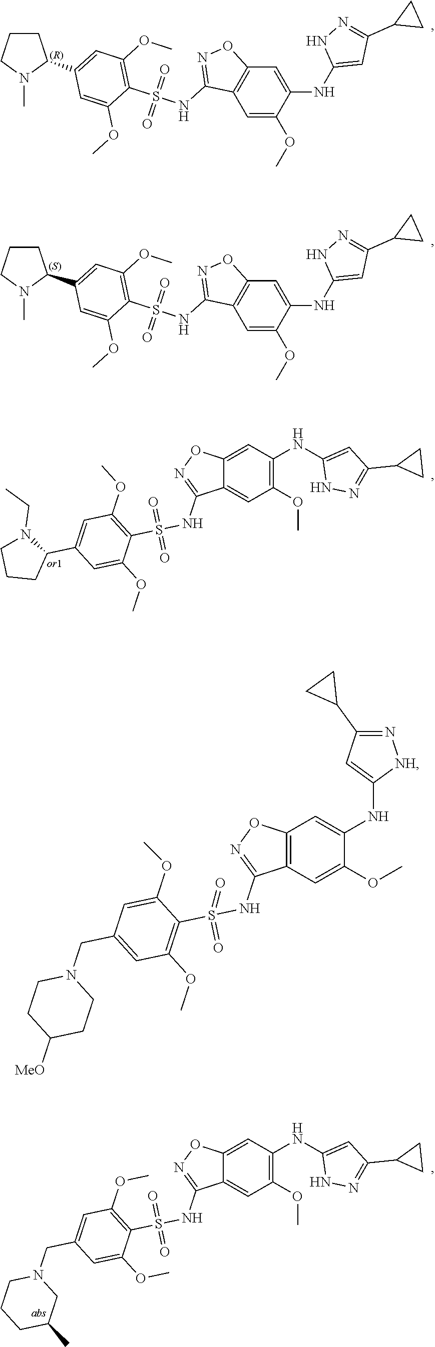 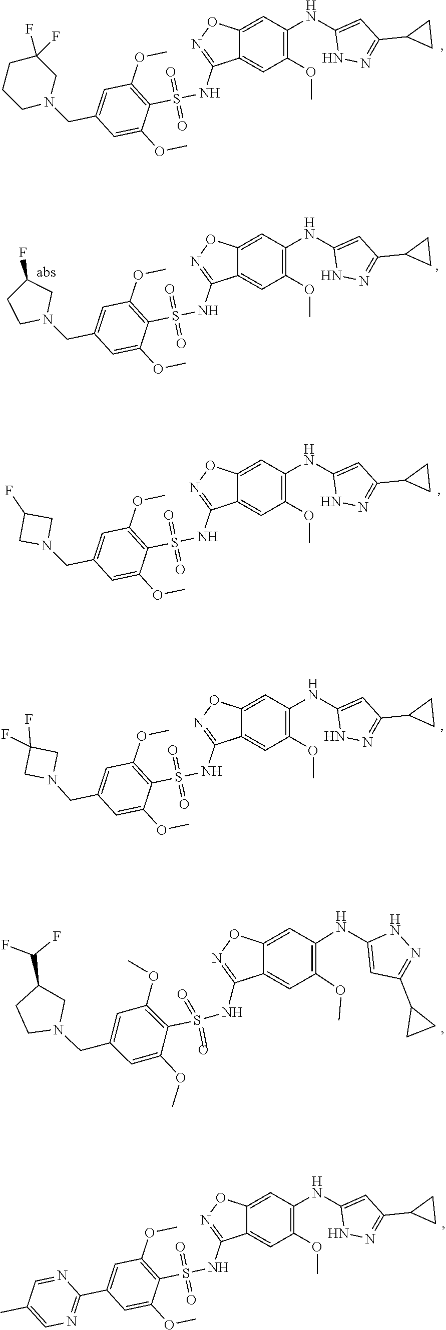 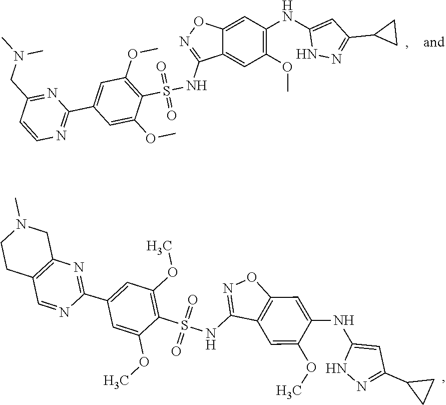 [SMILES: COc1cc2c(NS(=O)(=O)c3c(OC)cc(-c4nccc(CN(C)C)n4)cc3OC)noc2cc1Nc1cc(C2CC2)n[nH]1 ; COc1cc2c(NS(=O)(=O)c3c(OC)cc(-c4ncc5c(n4)CN(C)CC5)cc3OC)noc2cc1Nc1cc(C2CC2)n[nH]1]
or a tautomer thereof, or a pharmaceutically acceptable salt of the compound or the tautomer thereof.

12. The compound of claim 1, which is selected from the group consisting of 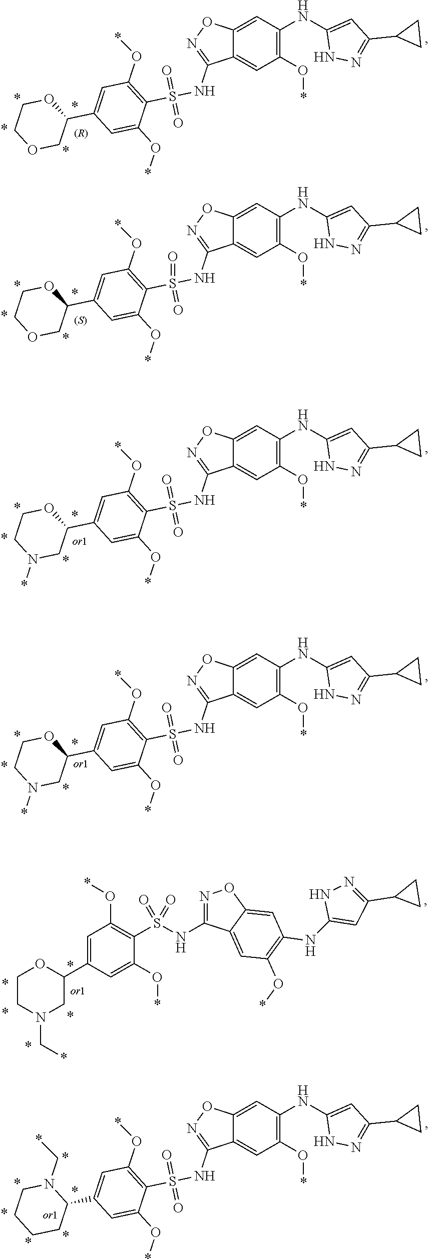 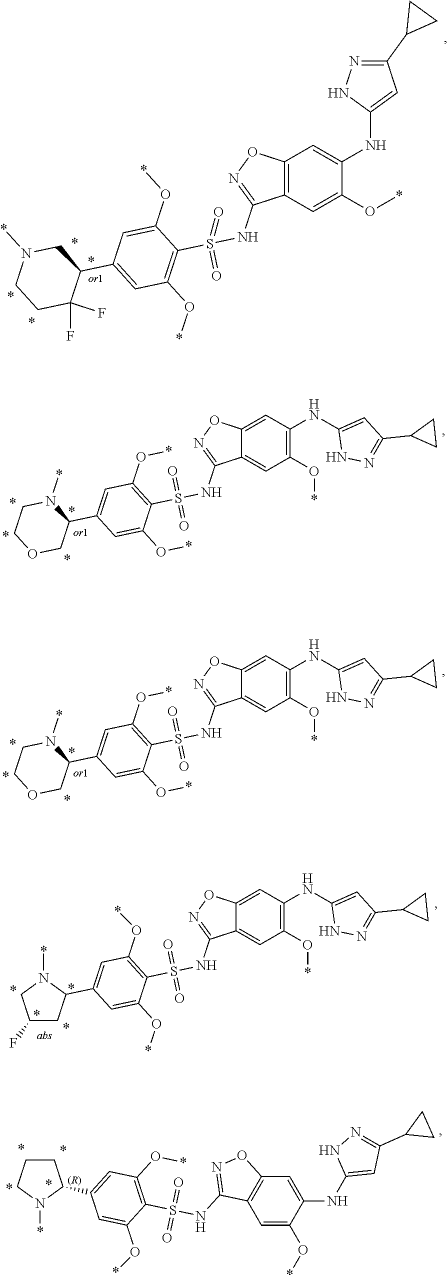 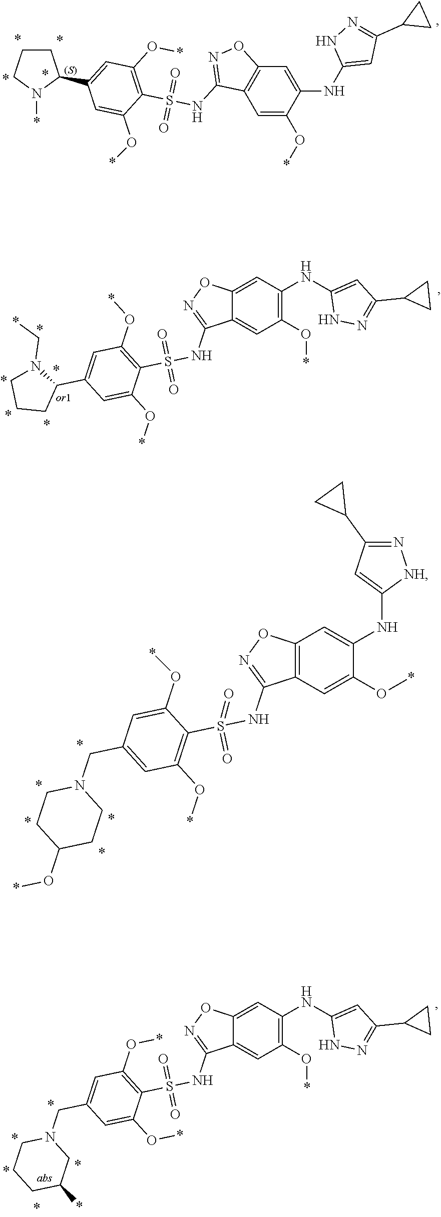 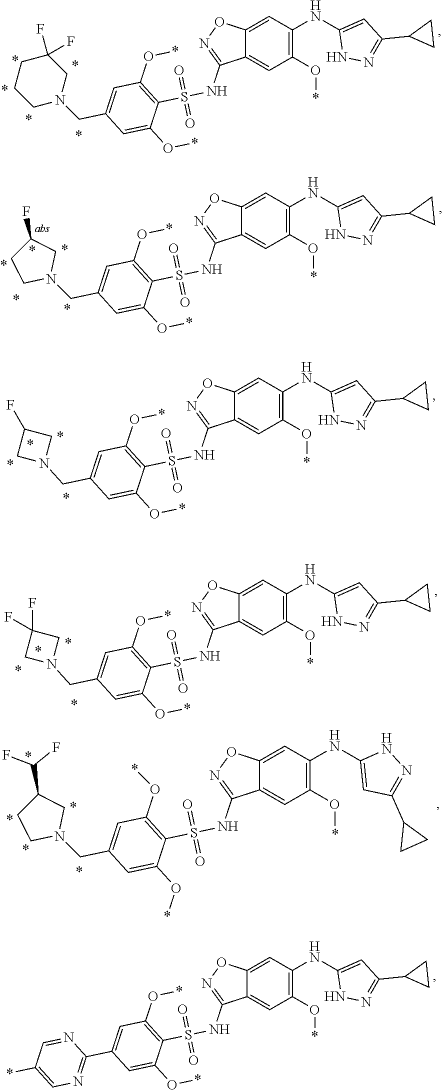 [SMILES: *Oc1cc2c(NS(=O)(=O)c3c(O*)cc(CN4CCCC(F)(F)C4)cc3O*)noc2cc1Nc1cc(C2CC2)n[nH]1 ; *Oc1cc2c(NS(=O)(=O)c3c(O*)cc(CN4CC[C@@H](F)C4)cc3O*)noc2cc1Nc1cc(C2CC2)n[nH]1 ; *Oc1cc2c(NS(=O)(=O)c3c(O*)cc(CN4CC(F)C4)cc3O*)noc2cc1Nc1cc(C2CC2)n[nH]1 ; *Oc1cc2c(NS(=O)(=O)c3c(O*)cc(CN4CC(F)(F)C4)cc3O*)noc2cc1Nc1cc(C2CC2)n[nH]1 ; *Oc1cc2c(NS(=O)(=O)c3c(O*)cc(CN4CC[C@@H](C(F)F)C4)cc3O*)noc2cc1Nc1cc(C2CC2)n[nH]1 ; *Oc1cc2c(NS(=O)(=O)c3c(O*)cc(-c4ncc(*)cn4)cc3O*)noc2cc1Nc1cc(C2CC2)n[nH]1] 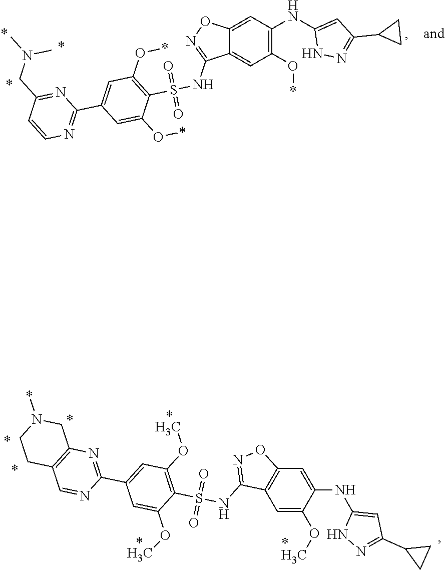 [SMILES: *Oc1cc2c(NS(=O)(=O)c3c(O*)cc(-c4nccc(CN(C)C)n4)cc3O*)noc2cc1Nc1cc(C2CC2)n[nH]1 ; *N1CCc2cnc(-c3cc(OC)c(S(=O)(=O)Nc4noc5cc(Nc6cc(C7CC7)n[nH]6)c(OC)cc45)c(OC)c3)nc2C1]
or a tautomer thereof, or a pharmaceutically acceptable salt of the compound or the tautomer thereof,
wherein * is a metabolic site, and
further wherein one or more hydrogen atoms of metabolic site are replaced by deuterium.

13. A compound, which is 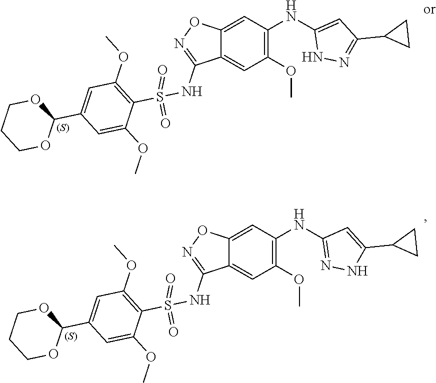
or a pharmaceutically acceptable salt thereof.

14. A compound, which is 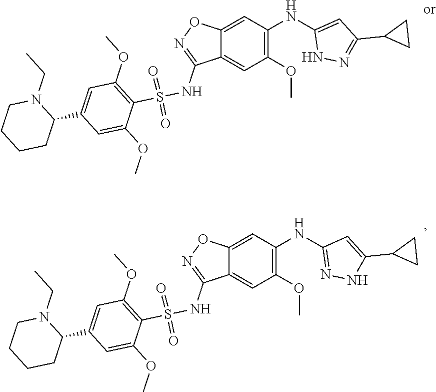 [SMILES: CCN1CCCC[C@H]1c1cc(OC)c(S(=O)(=O)Nc2noc3cc(Nc4cc(C5CC5)n[nH]4)c(OC)cc23)c(OC)c1 ; CCN1CCCC[C@H]1c1cc(OC)c(S(=O)(=O)Nc2noc3cc(Nc4cc(C5CC5)[nH]n4)c(OC)cc23)c(OC)c1]
or a pharmaceutically acceptable salt thereof.

15. A compound, which is 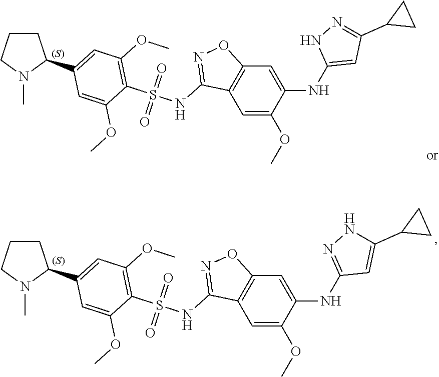
or a pharmaceutically acceptable salt thereof.

16. A pharmaceutical composition comprising the compound of claim 1, or a pharmaceutically acceptable salt thereof, and at least one pharmaceutically acceptable excipient.

17. A pharmaceutical composition comprising the compound of claim 11, or a pharmaceutically acceptable salt thereof, and at least one pharmaceutically acceptable excipient.

18. A pharmaceutical composition comprising the compound of claim 13, or a pharmaceutically acceptable salt thereof, and at least one pharmaceutically acceptable excipient.

19. A pharmaceutical composition comprising the compound of claim 14, or a pharmaceutically acceptable salt thereof, and at least one pharmaceutically acceptable excipient.

20. A pharmaceutical composition comprising the compound of claim 15, or a pharmaceutically acceptable salt thereof, and at least one pharmaceutically acceptable excipient.


SEQUENCE LISTING (11 sequences)

SEQ ID NO: 1 - protein, 21 aa, Homo sapiens
  source 1..21: note = Histone H3 (1-21) peptide
ARTKQTARKSTGGKAPRKQLA

SEQ ID NO: 2 - protein, 21 aa, Homo sapiens
  source 1..21: note = Histone H4 (1-21) peptide
SGRGKGGKGLGKGGAKRHRKV

SEQ ID NO: 3 - protein, 537 aa, synthetic construct
  source 1..537: note = N-terminal hexahistidine affinity tag linked to amino acid residues residues 1-513 (Uniprot Q92993-1) of human KAT5
MASHHHHHHDYDGATTENLYFQGSMAEVGEIIEGCRLPVLRRNQDNEDEWPLAEILSVKDISGRKLFYVHYIDFNKRLDEWVTHERLDLKKIQFPKKEAKTPTKNGLPGSRPGSPEREVPASAQASGKTLPIPVQITLRFNLPKEREAIPGGEPDQPLSSSSCLQPNHRSTKRKVEVVSPATPVPSETAPASVFPQNGAARRAVAAQPGRKRKSNCLGTDEDSQDSSDGIPSAPRMTGSLVSDRSHDDIVTRMKNIECIELGRHRLKPWYFSPYPQELTTLPVLYLCEFCLKYGRSLKCLQRHLTKCDLRHPPGNEIYRKGTISFFEIDGRKNKSYSQNLCLLAKCFLDHKTLYYDTDPFLFYVMTEYDCKGFHIVGYFSKEKESTEDYNVACILTLPPYQRRGYGKLLIEFSYELSKVEGKTGTPEKPLSDLGLLSYRSYWSQTILEILMGLKSESGERPQITINEISEITSIKKEDVISTLQYLNLINYYKGQYILTLSEDIVDGHERAMLKRLLRIDSKCLHFTPKDWSKRGKW

SEQ ID NO: 4 - protein, 2027 aa, synthetic construct
  source 1..2027: note = N-terminal Flag affinity Tag sequence linked to amino acid residues residues 1-2004 (Uniprot Q92794-1) of human KAT6A
MASDYKDDDDKGATTENLYFQGSMVKLANPLYTEWILEAIKKVKKQKQRPSEERICNAVSSSHGLDRKTVLEQLELSVKDGTILKVSNKGLNSYKDPDNPGRIALPKPRNHGKLDNKQNVDWNKLIKRAVEGLAESGGSTLKSIERFLKGQKDVSALFGGSAASGFHQQLRLAIKRAIGHGRLLKDGPLYRLNTKATNVDGKESCESLSCLPPVSLLPHEKDKPVAEPIPICSFCLGTKEQNREKKPEELISCADCGNSGHPSCLKFSPELTVRVKALRWQCIECKTCSSCRDQGKNADNMLFCDSCDRGFHMECCDPPLTRMPKGMWICQICRPRKKGRKLLQKKAAQIKRRYTNPIGRPKNRLKKQNTVSKGPFSKVRTGPGRGRKRKITLSSQSASSSSEEGYLERIDGLDFCRDSNVSLKFNKKTKGLIDGLTKFFTPSPDGRKARGEVVDYSEQYRIRKRGNRKSSTSDWPTDNQDGWDGKQENEERLFGSQEIMTEKDMELFRDIQEQALQKVGVTGPPDPQVRCPSVIEFGKYEIHTWYSSPYPQEYSRLPKLYLCEFCLKYMKSRTILQQHMKKCGWFHPPANEIYRKNNISVFEVDGNVSTIYCQNLCLLAKLFLDHKTLYYDVEPFLFYVLTQNDVKGCHLVGYFSKEKHCQQKYNVSCIMILPQYQRKGYGRFLIDFSYLLSKREGQAGSPEKPLSDLGRLSYMAYWKSVILECLYHQNDKQISIKKLSKLTGICPQDITSTLHHLRMLDFRSDQFVIIRREKLIQDHMAKLQLNLRPVDVDPECLRWTPVIVSNSVVSEEEEEEAEEGENEEPQCQERELEISVGKSVSHENKEQDSYSVESEKKPEVMAPVSSTRLSKQVLPHDSLPANSQPSRRGRWGRKNRKTQERFGDKDSKLLLEETSSAPQEQYGECGEKSEATQEQYTESEEQLVASEEQPSQDGKPDLPKRRLSEGVEPWRGQLKKSPEALKCRLTEGSERLPRRYSEGDRAVLRGFSESSEEEEEPESPRSSSPPILTKPTLKRKKPFLHRRRRVRKRKHHNSSVVTETISETTEVLDEPFEDSDSERPMPRLEPTFEIDEEEEEEDENELFPREYFRRLSSQDVLRCQSSSKRKSKDEEEDEESDDADDTPILKPVSLLRKRDVKNSPLEPDTSTPLKKKKGWPKGKSRKPIHWKKRPGRKPGFKLSREIMPVSTQACVIEPIVSIPKAGRKPKIQESEETVEPKEDMPLPEERKEEEEMQAEAEEAEEGEEEDAASSEVPAASPADSSNSPETETKEPEVEEEEEKPRVSEEQRQSEEEQQELEEPEPEEEEDAAAETAQNDDHDADDEDDGHLESTKKKELEEQPTREDVKEEPGVQESFLDANMQKSREKIKDKEETELDSEEEQPSHDTSVVSEQMAGSEDDHEEDSHTKEELIELKEEEEIPHSELDLETVQAVQSLTQEESSEHEGAYQDCEETLAACQTLQSYTQADEDPQMSMVEDCHASEHNSPISSVQSHPSQSVRSVSSPNVPALESGYTQISPEQGSLSAPSMQNMETSPMMDVPSVSDHSQQVVDSGFSDLGSIESTTENYENPSSYDSTMGGSICGNSSSQSSCSYGGLSSSSSLTQSSCVVTQQMASMGSSCSMMQQSSVQPAANCSIKSPQSCVVERPPSNQQQQPPPPPPQQPQPPPPQPQPAPQPPPPQQQPQQQPQPQPQQPPPPPPPQQQPPLSQCSMNNSFTPAPMIMEIPESGSTGNISIYERIPGDFGAGSYSQPSATFSLAKLQQLTNTIMDPHAMPYSHSPAVTSYATSVSLSNTGLAQLAPSHPLAGTPQAQATMTPPPNLASTTMNLTSPLLQCNMSATNIGIPHTQRLQGQMPVKGHISIRSKSAPLPSAAAHQQQLYGRSPSAVAMQAGPRALAVQRGMNMGVNLMPTPAYNVNSMNMNTLNAMNSYRMTQPMMNSSYHSNPAYMNQTAQYPMQMQMGMMGSQAYTQQPMQPNPHGNMMYTGPSHHSYMNAAGVPKQSLNGPYMRR

SEQ ID NO: 5 - protein, 625 aa, synthetic construct
  source 1..625: note = Amino acid residues 1-611 (Uniprot O95251-1) of human KAT7 linked to C-terminal hexahistidine affinity tag
MPRRKRNAGSSSDGTEDSDFSTDLEHTDSSESDGTSRRSARVTRSSARLSQSSQDSSPVRNLQSFGTEEPAYSTRRVTRSQQQPTPVTPKKYPLRQTRSSGSETEQVVDFSDRETKNTADHDESPPRTPTGNAPSSESDIDISSPNVSHDESIAKDMSLKDSGSDLSHRPKRRRFHESYNFNMKCPTPGCNSLGHLTGKHERHFSISGCPLYHNLSADECKVRAQSRDKQIEERMLSHRQDDNNRHATRHQAPTERQLRYKEKVAELRKKRNSGLSKEQKEKYMEHRQTYGNTREPLLENLTSEYDLDLFRRAQARASEDLEKLRLQGQITEGSNMIKTIAFGRYELDTWYHSPYPEEYARLGRLYMCEFCLKYMKSQTILRRHMAKCVWKHPPGDEIYRKGSISVFEVDGKKNKIYCQNLCLLAKLFLDHKTLYYDVEPFLFYVMTEADNTGCHLIGYFSKEKNSFLNYNVSCILTMPQYMRQGYGKMLIDFSYLLSKVEEKVGSPERPLSDLGLISYRSYWKEVLLRYLHNFQGKEISIKEISQETAVNPVDIVSTLQALQMLKYWKGKHLVLKRQDLIDEWIAKEAKRSNSNKTMDPSCLKWTPPKGTLENLYFQGHHHHHH

SEQ ID NO: 6 - protein, 482 aa, synthetic construct
  source 1..482: note = N-terminal hexahistidine affinity tag linked to amino acid residues 1-458 (Uniprot Q9H7Z6-1) of human KAT8
MASHHHHHHDYDGATTENLYFQGSMAAQGAAAAVAAGTSGVAGEGEPGPGENAAAEGTAPSPGRVSPPTPARGEPEVTVEIGETYLCRRPDSTWHSAEVIQSRVNDQEGREEFYVHYVGFNRRLDEWVDKNRLALTKTVKDAVQKNSEKYLSELAEQPERKITRNQKRKHDEINHVQKTYAEMDPTTAALEKEHEAITKVKYVDKIHIGNYEIDAWYFSPFPEDYGKQPKLWLCEYCLKYMKYEKSYRFHLGQCQWRQPPGKEIYRKSNISVYEVDGKDHKIYCQNLCLLAKLFLDHKTLYFDVEPFVFYILTEVDRQGAHIVGYFSKEKESPDGNNVACILTLPPYQRRGYGKFLIAFSYELSKLESTVGSPEKPLSDLGKLSYRSYWSWVLLEILRDFRGTLSIKDLSQMTSITQNDIISTLQSLNMVKYWKGQHVICVTPKLVEEHLKSAQYKKPPITVDSVCLKWAPPKHKQVKLSKK

SEQ ID NO: 7 - protein, 1237 aa, synthetic construct
  source 1..1237: note = N-terminal Flag affinity Tag sequence linked to amino acid residues 1-1214 (Uniprot P55201-1) of human BRPF1
MASDYKDDDDKGATTENLYFQGSMGVDFDVKTFCHNLRATKPPYECPVETCRKVYKSYSGIEYHLYHYDHDNPPPPQQTPLRKHKKKGRQSRPANKQSPSPSEVSQSPGREVMSYAQAQRMVEVDLHGRVHRISIFDNLDVVSEDEEAPEEAPENGSNKENTETPAATPKSGKHKNKEKRKDSNHHHHHNVSASTTPKLPEVVYRELEQDTPDAPPRPTSYYRYIEKSAEELDEEVEYDMDEEDYIWLDIMNERRKTEGVSPIPQEIFEYLMDRLEKESYFESHNKGDPNALVDEDAVCCICNDGECQNSNVILFCDMCNLAVHQECYGVPYIPEGQWLCRRCLQSPSRAVDCALCPNKGGAFKQTDDGRWAHVVCALWIPEVCFANTVFLEPIDSIEHIPPARWKLTCYICKQRGSGACIQCHKANCYTAFHVTCAQQAGLYMKMEPVRETGANGTSFSVRKTAYCDIHTPPGSARRLPALSHSEGEEDEDEEEDEGKGWSSEKVKKAKAKSRIKMKKARKILAEKRAAAPVVSVPCIPPHRLSKITNRLTIQRKSQFMQRLHSYWTLKRQSRNGVPLLRRLQTHLQSQRNCDQVGRDSEDKNWALKEQLKSWQRLRHDLERARLLVELIRKREKLKRETIKVQQIAMEMQLTPFLILLRKTLEQLQEKDTGNIFSEPVPLSEVPDYLDHIKKPMDFFTMKQNLEAYRYLNFDDFEEDFNLIVSNCLKYNAKDTIFYRAAVRLREQGGAVLRQARRQAEKMGIDFETGMHIPHSLAGDEATHHTEDAAEEERLVLLENQKHLPVEEQLKLLLERLDEVNASKQSVGRSRRAKMIKKEMTALRRKLAHQRETGRDGPERHGPSSRGSLTPHPAACDKDGQTDSAAEESSSQETSKGLGPNMSSTPAHEVGRRTSVLFSKKNPKTAGPPKRPGRPPKNRESQMTPSHGGSPVGPPQLPIMSSLRQRKRGRSPRPSSSSDSDSDKSTEDPPMDLPANGFSGGNQPVKKSFLVYRNDCSLPRSSSDSESSSSSSSSAASDRTSTTPSKQGRGKPSFSRGTFPEDSSEDTSGTENEAYSVGTGRGVGHSMVRKSLGRGAGWLSEDEDSPLDALDLVWAKCRGYPSYPALIIDPKMPREGMFHHGVPIPVPPLEVLKLGEQMTQEAREHLYLVLFFDNKRTWQWLPRTKLVPLGVNQDLDKEKMLEGRKSNIRKSVQIAYHRALQHRSKVQGEQSSETSDSD

SEQ ID NO: 8 - protein, 1080 aa, synthetic construct
  source 1..1080: note = N-terminal Flag affinity Tag sequence linked to amino acid residues 2-1058 (Uniprot O95696-1) of human BRPF2
MASDYKDDDDKGATTENLYFQGSRRKGRCHRGSAARHPSSPCSVKHSPTRETLTYAQAQRMVEIEIEGRLHRISIFDPLEIILEDDLTAQEMSECNSNKENSERPPVCLRTKRHKNNRVKKKNEALPSAHGTPASASALPEPKVRIVEYSPPSAPRRPPVYYKFIEKSAEELDNEVEYDMDEEDYAWLEIVNEKRKGDCVPAVSQSMFEFLMDRFEKESHCENQKQGEQQSLIDEDAVCCICMDGECQNSNVILFCDMCNLAVHQECYGVPYIPEGQWLCRHCLQSRARPADCVLCPNKGGAFKKTDDDRWGHVVCALWIPEVGFANTVFIEPIDGVRNIPPARWKLTCYLCKQKGVGACIQCHKANCYTAFHVTCAQKAGLYMKMEPVKELTGGGTTFSVRKTAYCDVHTPPGCTRRPLNIYGDVEMKNGVCRKESSVKTVRSTSKVRKKAKKAKKALAEPCAVLPTVCAPYIPPQRLNRIANQVAIQRKKQFVERAHSYWLLKRLSRNGAPLLRRLQSSLQSQRSSQQRENDEEMKAAKEKLKYWQRLRHDLERARLLIELLRKREKLKREQVKVEQVAMELRLTPLTVLLRSVLDQLQDKDPARIFAQPVSLKEVPDYLDHIKHPMDFATMRKRLEAQGYKNLHEFEEDFDLIIDNCMKYNARDTVFYRAAVRLRDQGGVVLRQARREVDSIGLEEASGMHLPERPAAAPRRPFSWEDVDRLLDPANRAHLGLEEQLRELLDMLDLTCAMKSSGSRSKRAKLLKKEIALLRNKLSQQHSQPLPTGPGLEGFEEDGAALGPEAGEEVLPRLETLLQPRKRSRSTCGDSEVEEESPGKRLDAGLTNGFGGARSEQEPGGGLGRKATPRRRCASESSISSSNSPLCDSSFNAPKCGRGKPALVRRHTLEDRSELISCIENGNYAKAARIAAEVGQSSMWISTDAAASVLEPLKVVWAKCSGYPSYPALIIDPKMPRVPGHHNGVTIPAPPLDVLKIGEHMQTKSDEKLFLVLFFDNKRSWQWLPKSKMVPLGIDETIDKLKMMEGRNSSIRKAVRIAFDRAMNHLSRVHGEPTSDLSDID

SEQ ID NO: 9 - protein, 865 aa, synthetic construct
  source 1..865: note = N-terminal Flag affinity Tag sequence linked to amino acid residues 1-842 (Uniprot Q6IE81-1) of human Jade1
MASDYKDDDDKGATTENLYFQGSMKRGRLPSSSEDSDDNGSLSTTWSQNSRSQHRRSSCSRHEDRKPSEVFRTDLITAMKLHDSYQLNPDEYYVLADPWRQEWEKGVQVPVSPGTIPQPVARVVSEEKSLMFIRPKKYIVSSGSEPPELGYVDIRTLADSVCRYDLNDMDAAWLELTNEEFKEMGMPELDEYTMERVLEEFEQRCYDNMNHAIETEEGLGIEYDEDVVCDVCQSPDGEDGNEMVFCDKCNICVHQACYGILKVPEGSWLCRTCALGVQPKCLLCPKKGGAMKPTRSGTKWVHVSCALWIPEVSIGSPEKMEPITKVSHIPSSRWALVCSLCNEKFGASIQCSVKNCRTAFHVTCAFDRGLEMKTILAENDEVKFKSYCPKHSSHRKPEESLGKGAAQENGAPECSPRNPLEPFASLEQNREEAHRVSVRKQKLQQLEDEFYTFVNLLDVARALRLPEEVVDFLYQYWKLKRKVNFNKPLITPKKDEEDNLAKREQDVLFRRLQLFTHLRQDLERVRNLTYMVTRREKIKRSVCKVQEQIFNLYTKLLEQERVSGVPSSCSSSSLENMLLFNSPSVGPDAPKIEDLKWHSAFFRKQMGTSLVHSLKKPHKRDPLQNSPGSEGKTLLKQPDLCGRREGMVVPESFLGLEKTFAEARLISAQQKNGVVMPDHGKRRDNRFHCDLIKGDLKDKSFKQSHKPLRSTDVSQRHLDNTRAATSPGVGQSAPGTRKEIVPKCNGSLIKVNYNQTAVKVPTTPASPVKNWGGFRIPKKGERQQQGEAHDGACHQHSDYPYLGLGRVPAKERAKSKLKSDNENDGYVPDVEMSDSESEASEKKCIHTSSTISRRTDIIRRSILAS

SEQ ID NO: 10 - protein, 240 aa, Homo sapiens
  source 1..240: note = Amino acid residues 1-240 (Uniprot Q8WYH8-1) of human ING5
MATAMYLEHYLDSIENLPCELQRNFQLMRELDQRTEDKKAEIDILAAEYISTVKTLSPDQRVERLQKIQNAYSKCKEYSDDKVQLAMQTYEMVDKHIRRLDADLARFEADLKDKMEGSDFESSGGRGLKKGRGQKEKRGSRGRGRRTSEEDTPKKKKHKGGSEFTDTILSVHPSDVLDMPVDPNEPTYCLCHQVSYGEMIGCDNPDCPIEWFHFACVDLTTKPKGKWFCPRCVQEKRKKK

SEQ ID NO: 11 - protein, 199 aa, Homo sapiens
  source 1..199: note = Amino acid residues 3-201 (Uniprot Q9HAF1-3) of human MEAF6
MHNKAAPPQIPDTRRELAELVKRKQELAETLANLERQIYAFEGSYLEDTQMYGNIIRGWDRYLTNQKNSNSKNDRRNRKFKEAERLFSKSSVTSAAAVSALAGVQDQLIEKREPGSGTESDTSPDFHNQENEPSQEDPEDLDGSVQGVKPQKAASSTSSGSHHSSHKKRKNKNRHSPSGMFDYDFEIDLKLNKKPRADY